(19) United States
(12) Patent Application Publication
(10) Pub. No.: US 2004/0009549 A1
(43) Pub. Date: Jan. 15, 2004

(54) Method for detecting remote homologues and novel kinases identified with the method

(75) Inventors: Igor Vyacheslavovich Grigoriev, Berkeley, CA (US); Sucha Sudarsanam, Greenbrae, CA (US)

Correspondence Address: FOLEY AND LARDNER, SUITE 500, 3000 K STREET NW, WASHINGTON, DC, 20007, US

(73) Assignee: Sugen, Inc.

(21) Appl. No.: 10/334,143

(22) Filed: Dec. 31, 2002

Related U.S. Application Data
(60) Provisional application No. 60/343,169, filed on Dec. 31, 2001.

(51) Int. Cl. C12P 21/02; C12N 5/06; C07H 21/04; C12N 9/12

(52) U.S. Cl. 435/69.1; 435/194; 435/320.1; 435/325; 536/23.2

(57) ABSTRACT

The present invention relates to novel methods for detecting remote polypeptide homologues. The present invention relates to kinase polypeptides, nucleotide sequences encoding the kinase polypeptides, as well as various products and methods useful for the diagnosis and treatment of various kinase-related diseases and conditions. Through the use of a bioinformatics strategy, mammalian kinases have been identified and their protein structure predicted.

The present invention is related to U.S. provisional application No. 60/343,169, filed Dec. 31, 2001, which is hereby incorporated by reference.

### FIELD OF THE INVENTION

The present invention relates to novel methods for detecting remote polypeptide homologues. The present invention also relates to novel kinase polypeptides identified using these novel methods, nucleotide sequences encoding the kinase polypeptides, as well as various products and methods useful for the diagnosis and treatment of various kinase-related diseases and conditions.

### BACKGROUND OF THE INVENTION

The following description of the background of the invention is provided to aid in understanding the invention, but is not admitted to be or to describe prior art to the invention.

About half the proteins encoded by completely sequenced genomes, including the human genome, are of unknown function. Homology methods, which are traditionally used to infer functions of unknown proteins, fail when the sequence similarity is too low. On the other hand, evolutionary relationships between functionally similar proteins can be often seen only by comparison of their secondary and tertiary structures, when there is as little as 5% residue identity between the sequences.

The protein threading approach for prediction of protein function is known in the art, and uses empirical energy potentials to align protein sequences with sets of three-dimensional (3D) coordinates of atoms from known protein structures. See Bowie J U, et al. (1991) Science. 253(5016):164-70; Jones D T, et al. (1992) 358(6381):86-9. Faster 1D protein threading techniques approximate 3D protein folds using simultaneous alignment of amino acid residues and their predicted secondary structure conformations. See Russel, et al. Fischer, et al. Grigoriev et al (references in REFERENCE list).

In contrast to protein tertiary structure, secondary structure takes into consideration only local interactions between the residues next to each other in sequence (one-dimensional, not three-dimensional, space). Secondary structure also excludes spatial contacts between sequentially distant residues. Nevertheless, secondary structure pattern can describe protein folding to some extent. See Sheridan R P Int. J. Peptide Protein Res. 25:132-143; and Aurora et al. list of references below. However, because different folds may have the same pattern (e.g., all-alpha or all-beta proteins), inferring fold similarities solely from secondary structure alignments can be misleading.

It is known in the art that certain amino acid residues, generally highly conserved during evolution, are critical for protein function. Several studies have been conducted to derive 3D patterns (residue identities and distances between them) of active sites from known structures, and then use these 3D patterns to predict function of a protein, but only after the protein 3D structure is available. See Skolnick J, Fetrow J S. (2000) Trends Biotechnol. January 2000;18(1):34-9; Thornton et al. list of references, and Reddy et al. Proteins 42(2):148-163. However, such 3D patterns cannot be used for functional annotation of a novel gene in absence of 3D structure. Moreover, these residues usually do not have clear local sequence context. Additionally, sequence patterns of active sites (residue identities only) show very low selectivity because of the small number of residues involved in the pattern as well as large and variable sequential separation between the residues.

Cellular signal transduction is a fundamental mechanism whereby external stimuli that regulate diverse cellular processes are relayed to the interior of cells. One of the key biochemical mechanisms of signal transduction involves the reversible phosphorylation of proteins, which enables regulation of the activity of mature proteins by altering their structure and function.

Protein phosphorylation plays a pivotal role in cellular signal transduction. Among the biological functions controlled by this type of postranslational modification are: cell division, differentiation and death (apoptosis); cell motility and cytoskeletal structure; control of DNA replication, transcription, splicing and translation; protein translocation events from the endoplasmic reticulum and Golgi apparatus to the membrane and extracellular space; protein nuclear import and export; regulation of metabolic reactions, etc. Abnormal protein phosphorylation is widely recognized to be causally linked to the etiology of many diseases including cancer as well as immunologic, neuronal and metabolic disorders.

The following abbreviations are used for kinases throught this application:

| | |
|---|---|
| ASK | Apoptosis signal-regulating kinase |
| CaMK | Ca2+/calmodulin-dependent protein kinase |
| CCRK | Cell cycle-related kinase |
| CDK | Cyclin-dependent kinase |
| CK | Casein kinase |
| DAPK | Death-associated protein kinase |
| DM | myotonic dystrophy kinase |
| DyrK | dual-specificity-tyrosine phosphorylating-regulated |
| | kinase |
| GAK | Cyclin G-associated kinase |
| GRK | G-protein coupled receptor |
| GuC | Guanylate cyclase |
| HIPK | Homeodomain-interacting protein kinase |
| IRAK | Interleukin-1 receptor-associated kinase |
| MAPK | Mitogen activated protein kinase |
| MAST | Microtubule-associated STK |
| MLCK | Myosin-light chain kinase |
| MLK | Mixed lineage kinase |
| NIMA | NimA-related protein kinase |
| PKA | cAMP-dependent protein kinase |
| RSK | Ribosomal protein S6 kinase |
| RTK | Receptor tyrosine kinase |
| SGK | Serum and glucocorticoid-regulated kinase |
| STK | serine threonine kinase |
| ULK | UNC-51-like kinase |

The best-characterized protein kinases in eukaryotes phosphorylate proteins on the hydroxyl substituent of serine, threonine and tyrosine residues, which are the most common phospho-acceptor amino acid residues. However, phosphorylation on histidine has also been observed in bacteria.

The presence of a phosphate moiety modulates protein function in multiple ways. A common mechanism includes changes in the catalytic properties (Vmax and Km) of an enzyme, leading to its activation or inactivation.

A second widely recognized mechanism involves promoting protein-protein interactions. An example of this is the tyrosine autophosphorylation of the ligand-activated EGF receptor tyrosine kinase. This event triggers the high-affinity binding to the phosphotyrosine residue on the receptor's C-terminal intracellular domain to the SH2 motif of the adaptor molecule Grb2. Grb2, in turn, binds through its SH3 motif to a second adaptor molecule, such as SHC. The formation of this ternary complex activates the signaling events that are responsible for the biological effects of EGF. Serine and threonine phosphorylation events also have been recently recognized to exert their biological function through protein-protein interaction events that are mediated by the high-affinity binding of phosphoserine and phosphothreonine to WW motifs present in a large variety of proteins (Lu, P. J. et al (1999) Science 283:1325-1328).

A third important outcome of protein phosphorylation is changes in the subcellular localization of the substrate. As an example, nuclear import and export events in a large diversity of proteins are regulated by protein phosphorylation (Drier E. A. et al (1999) Genes Dev 13: 556-568).

Protein kinases are one of the largest families of eukaryotic proteins with several hundred known members. These proteins share a 250-300 amino acid domain that can be subdivided into 12 distinct subdomains that comprise the common catalytic core structure. These conserved protein motifs have recently been exploited using PCR-based and bioinformatic strategies leading to a significant expansion of the known kinases. Multiple alignment of the sequences in the catalytic domain of protein kinases and subsequent parsimony analysis permits their segregation into sub-families of related kinases.

Kinases largely fall into two groups: those specific for phosphorylating serines and threonines, and those specific for phosphorylating tyrosines. Some kinases, referred to as “dual specificity” kinases, are able to phosphorylate on tyrosine as well as serine/threonine residues.

Protein kinases can also be characterized by their location within the cell. Some kinases are transmembrane receptor-type proteins capable of directly altering their catalytic activity in response to the external environment such as the binding of a ligand. Others are non-receptor-type proteins lacking any transmembrane domain. They can be found in a variety of cellular compartments from the inner surface of the cell membrane to the nucleus.

Many kinases are involved in regulatory cascades wherein their substrates may include other kinases whose activities are regulated by their phosphorylation state. Ultimately the activity of some downstream effector is modulated by phosphorylation resulting from activation of such a pathway. The conserved protein motifs of these kinases have recently been exploited using PCR-based cloning strategies leading to a significant expansion of the known kinases.

Multiple alignment of the sequences in the catalytic domain of protein kinases and subsequent parsimony analysis permits the segregation of related kinases into distinct branches of subfamilies including: tyrosine kinases (PTK's), dual-specificity kinases, and serine/threonine kinases (STK's). The latter subfamily includes cyclic-nucleotide-dependent kinases, calcium/calmodulin kinases, cyclin-dependent kinases (CDK's), MAP-kinases, serine-threonine kinase receptors, and several other less defined subfamilies.

The protein kinases may be classified into several major groups including AGC, CAMK, Casein kinase 1, CMGC, STE, tyrosine kinases, and a typical kinases (Plowman, G D et al., Proceedings of the National Academy of Sciences, USA, Vol. 96, Issue 24, 13603-13610, Nov. 23, 1999; Manning, et al. Trends Biochem. Sci. 27(10)514 (2002); Manning, et al. Science 298:1912 (2002); see also www.kinase.com). In addition, there are a number of minor yet distinct families, including families related to worm- or fungal-specific kinases, and a family designated “other” to represent several smaller families. Within each group are several distinct families of more closely related kinases. Members of these families have been shown to be associated with various diseases. In addition, an “a typical” family represents those protein kinases whose catalytic domain has little or no primary sequence homology to conventional kinases, including the PI3 kinases.

AGC Group

The AGC kinases are basic amino acid-directed enzymes that phosphorylate residues found proximal to Arg and Lys. Examples of this group are the G protein-coupled receptor kinases (GRKs), the cyclic nucleotide-dependent kinases (PKA, PKC, PKG), NDR or DBF2 kinases, ribosomal S6 kinases, AKT kinases, myotonic dystrophy kinases (DMPKs), MAPK interacting kinases (MNKs), MAST kinases, and Mo3C11.1_ce family originally identified only in nematodes.

GRKs regulate signaling from heterotrimeric guanine protein coupled receptors (GPCRs). Mutations in GPCRs cause a number of human diseases, including retinitis pigmentosa, stationary night blindness, color blindness, hyperfunctioning thyroid adenomas, familial precocious puberty, familial hypocalciuric hypercalcemia and neonatal severe hyperparathroidism (OMIM, http://www.ncbi.nlm.nih.gov/Omim/). The regulation of GPCRs by GRKs indirectly implicates GRKs in these diseases.

The cAMP-dependent protein kinases (PKA) consist of heterotetramers comprised of 2 catalytic (C) and 2 regulatory (R) subunits, in which the R subunits bind to the second messenger cAMP, leading to dissociation of the active C subunits from the complex. Many of these kinases respond to second messengers such as cAMP resulting in a wide range of cellular responses to hormones and neurotransmitters.

AKT is a mammalian proto-oncoprotein regulated by phosphatidylinositol 3-kinase (PI3-K), which appears to function as a cell survival signal to protect cells from apoptosis. Insulin receptor, RAS, PI3-K, and PDK1 all act as upstream activators of AKT, whereas the lipid phosphatase PTEN functions as a negative regulator of the PI3-K/AKT pathway. Downstream targets for AKT-mediated cell survival include the pro-apoptotic factors BAD and Caspase9, and transcription factors in the forkhead family, such as DAF-16 in the worm. AKT is also an essential mediator in insulin signaling, in part due to its use of GSK-3 as another downstream target.

The S6 kinases regulate a wide array of cellular processes involved in mitogenic response including protein synthesis, translation of specific mRNA species, and cell cycle progression from G1 to S phase. The gene has been localized to chromosomal region 17q23 and is amplified in breast cancer (Couch, et al., Cancer Res. Apr. 1, 1999;59(7):1408-11).

CAMK Group

The CAMK kinases are also basic amino acid-directed kinases. They include the Ca2+/calmodulin-regulated and AMP-dependent protein kinases (AMPK), myosin light chain kinases (MLCK), MAP kinase activating protein kinases (MAPKAPKs) checkpoint 2 kinases (CHK2), death-associated protein kinases (DAPKs), phosphorylase kinase (PHK), Rac and Rho-binding Trio kinases, a “unique” family of CAMKs, and the EMK-related protein kinases.

The EMK family of STKs are involved in the control of cell polarity, microtubule stability and cancer. One member of the EMK family, C-TAK1, has been reported to control entry into mitosis by activating Cdc25C which in turn dephosphorylates Cdc2. Also included in the EMK family is MAKV, which has been shown to be overexpressed in metastatic tumors (Dokl. Akad. Nauk 354 (4), 554-556 (1997)).

CMGC Group

The CMGC kinases are “proline-directed” enzymes 20, phosphorylating residues that exist in a proline-rich context. They include the cyclin-dependent kinases (CDKs), mitogen-activated protein kinases (MAPKs), GSK3s, RCKs, and CLKs. Most CMGC kinases have larger-than-average kinase domains owing to the presence of insertions within subdomains X and XI.

CDK's play a pivotal role in the regulation of mitosis during cell division. The process of cell division occurs in four stages: S phase, the period during which chromosomes duplicate, G2, mitosis and G1 or interphase. During mitosis the duplicated chromosomes are evenly segregated allowing each daughter cell to receive a complete copy of the genome. A key mitotic regulator in all eukaryotic cells is the STK cdc2, a CDK regulated by cyclin B. However some CDK-like kinases, such as CDK5 are not cyclin associated nor are they cell cycle regulated.

MAPKs play a pivotal role in many cellular signaling pathways, including stress response and mitogenesis (Lewis, T. S., Shapiro, P. S., and Ahn, N. G. (1998) Adv. Cancer Res. 74, 49-139). MAP kinases can be activated by growth factors such as EGF, and cytokines such as TNF-alpha. In response to EGF, Ras becomes activated and recruits Raf1 to the membrane where Raf1 is activated by mechanisms that may involve phosphorylation and conformational changes (Morrison, D. K., and Cutler, R. E. (1997) Curr. Opin. Cell Biol. 9, 174-179). Active Raf1 phosphorylates MEK1 which in turn phosphorylates and activates the ERKs.

Tyrosine Protein Kinase Group

The tyrosine kinase group encompass both cytoplasmic (e.g. src) as well as transmembrane receptor tyrosine kinases (e.g. EGF receptor). These kinases play a pivotal role in the signal transduction processes that mediate cell proliferation, differentiation and apoptosis. Mutations of the RET gene, encoding a receptor tyrosine kinase, have been associated with the inherited cancer syndromes MEN 2A and MEN 2B. They have also further been associated with both familial and sporadic medullary thyroid carcinomas. The kinase activity can be aberrantly activated by missense mutations affecting cysteine residues within the extracellular domain, leading to potent oncogenicity (Oncogene Aug. 26, 1999;18(34):4833-8).

STE Group

The STE family refers to the 3 classes of protein kinases that lie sequentially upstream of the MAPKs. This group includes STE7 (MEK or MAPKK) kinases, STE11 (MEKK or MAPKKK) kinases and STE20 (MEKKK) kinases. In humans, several protein kinase families that bear only distant homology with the STE11 family also operate at the level of MAPKKKs including RAF, MLK, TAK1, and COT. Since crosstalk takes place between protein kinases functioning at different levels of the MAPK cascade, the large number of STE family kinases could translate into an enormous potential for upstream signal specificity.

The prototype STE20 from baker's yeast is regulated by a hormone receptor, signaling to directly affect cell cycle progression through modulation of CDK activity. It also coordinately regulates changes in the cytoskeleton and in transcriptional programs in a bifurcating pathway. In a similar way, the homologous kinases in humans are likely to play a role in extracellular regulation of growth, cell adhesion and migration, and changes in transcriptional programs, all three of which have critical roles in tumorigenesis. Mammalian STE20-related protein kinases have been implicated in response to growth factors or cytokines, oxidative-, UV-, or irradiation-related stress pathways, inflammatory signals (e.g. TNFα), apoptotic stimuli (e.g. Fas), T and B cell costimulation, the control of cytoskeletal architecture, and cellular transformation. Typically the STE20-related kinases serve as upstream regulators of MAPK cascades. Examples include: HPK1, a protein-serine/threonine kinase (STK) that possesses a STE20-like kinase domain that activates a protein kinase pathway leading to the stress-activated protein kinase SAPK/JNK; PAK1, an STK with an upstream CDC42-binding domain that interacts with Rac and plays a role in cellular transformation through the Ras-MAPK pathway; and murine NIK, which interacts with upstream receptor tyrosine kinases and connects with downstream STE11-family kinases.

NEK kinases are related to NIMA, which is required for entry into mitosis in the filamentous fungus A. nidulans. Mutations in the nima gene cause the nim (never in mitosis) G2 arrest phenotype in this fungus (Fry, A. M. and Nigg, E. A. (1995) Current Biology 5: 1122-1125). Several observations suggest that higher eukaryotes may have a NIMA functional counterpart(s): (1) expression of a dominant-negative form of NIMA in HeLa cells causes a G2 arrest; (2)overexpression of NIMA causes chromatin condensation, not only in A. nidulans, but also in yeast, Xenopus oocytes and HeLa cells (Lu, K. P. and Hunter, T. (1995) Prog. Cell Cycle Res. 1, 187-205); (3) NIMA when expressed in mammalian cells interacts with pin1, a prolyl-prolyl isomerase that functions in cell cycle regulation (Lu, K. P. et al. (1996) Nature 380, 544-547); (4) okadaic acid inhibitor studies suggests the presence of cdc2-independent mechanism to induce mitosis (Ghosh, S. et al.(1998) Exp. Cell Res. 242, 1-9) and (5) a NIMA-like kinase (fin1) exists in another eukaryote besides Aspergillus, Saccharomyces pombe (Krien, M. J. E. et al.(1998) J. Cell Sci. 111, 967-976). Four mammalian NIMA-like kinases have been identified. NEK1, NEK2, NEK3 and NRK2. Despite the similarity of the NIMA-related kinases to NIMA over the catalytic region, the mammalian kinases are structurally different to NIMA over the extracatalytic regions.

Casein Kinase 1 Group

The CK1 family represents a distant branch of the protein kinase family. The hallmarks of protein kinase subdomains VIII and IX are difficult to identify. One or more forms are ubiquitously distributed in mammalian tissues and cell lines. CK1 kinases are found in cytoplasm, in nuclei, membrane-bound, and associated with the cytoskeleton. Splice variants differ in their subcellular distribution.

“Other” Group

Several families cluster within a group of unrelated kinases termed “Other”. Included are: CHK1; Elongation 2 factor kinases (EIFK); homologues of the yeast sterile family kinases (STE), which refers to 3 classes of kinases which lie sequentially upstream of the MAPKs; Calcium-calmodulin kinase kinases (CAMKK); dual-specific tyrosine kinases (DYRK); IkB kinases (IKK); Integrin receptor kinase (IRAK); endoribonuclease-associated kinases (IRE); Mixed lineage kinase (MLK); LIM-domain containing kinase (LIMK); MOS; PIM; Receptor interacting kinase (RIP); SR-protein specific kinase (SRPK); RAF; Serine-threonine kinase receptors (STKR); TAKI; Testis specific kinase (TSK); tousled-related kinase (TSL); UNC51-related kinase (UNC); VRK; WEE; mitotic kinases (BUBI, AURORA, PLK, and NIMA/NEK); several families that are close homologues to worm (C26C2.1, YQO9, ZC581.9, YFLO33c, C24A1.3); Drosophila (SLOB), or yeast (YDOD_sp, YGR262_sc) kinases; and others that are “unique,” that is, those which do not cluster into any obvious family. Additional families are even less well defined and first were identified in lower eukaryotes such as yeast or worms (YNL020, YPL236, YQ09, YWY3, SCY1, C01H6.9, C26C2.1)

RIP2 is a serine-threonine kinase associated with the tumor necrosis factor (TNF) receptor complex and is implicated in the activation of NF-kappa B and cell death in mammalian cells. It has recently been demonstrated that RIP2 activates the MAPK pathway (Navas, et al., J. Biol. Chem. Nov. 19, 1999;274(47):33684-33690). RIP2 activates AP-1 and serum response element regulated expression by inducing the activation of the Elk1 transcription factor. RIP2 directly phosphorylates and activates ERK2 in vivo and in vitro. RIP2 in turn is activated through its interaction with Ras-activated Raf1. These results highlight the integrated nature of kinase signaling pathway.

The tousled (TSL) kinase was first identified in the plant Arabidopsis thaliana. TSL encodes a serine/threonine kinase that is essential for proper flower development. Human tousled-like kinases (Tlks) are cell-cycle-regulated enzymes, displaying maximal activities during S phase. This regulated activity suggests that Tlk function is linked to ongoing DNA replication (Sillje, et al., EMBO J. Oct. 15, 1999;18(20):5691-5702).

Atypical Protein Kinase Group

There are several proteins with protein kinase activity that do not show any significant homology to the eukaryotic protein kinases. These include, for example, Dictyostelium myosin heavy chain kinase A (MHCKA) and Physarum polycephalum actin-fragmin kinase. The slime mold, worm and human eEF-2 kinase homologues have all been demonstrated to have protein kinase activity, yet they bear little resemblance to conventional protein kinases on the sequece level except for the presence of a putative GxGxxG ATP-binding motif.

Several other proteins contain protein kinase-like homology including: receptor guanylyl cyclases, diacylglycerol kinases, choline/ethanolamine kinases, and YLK1-related antibiotic resistance kinases. Each of these families contain short motifs that were recognized by our profile searches with low scoring E-values, but a priori would not be expected to function as protein kinases. Instead, the similarity could simply reflect the modular nature of protein evolution and the primal role of ATP binding in diverse phosphotransfer enzymes. However, two recent papers on a bacterial homologue of the YLK1 family suggests that the aminoglycoside phosphotransferases (APHs) are structurally and functionally related to protein kinases. There are over 40 APHs identified from bacteria that are resistant to aminoglycosides such as kanamycin, gentamycin, or amikacin. The crystal structure of one well characterized APH reveals that it shares greater than 40% structural identity with the 2 lobed structure of the catalytic domain of cAMP-dependent protein kinase (PKA), including an N-terminal lobe composed of a 5-stranded antiparallel beta sheet and the core of the C-terminal lobe including several invariant segments found in all protein kinases. APHs lack the GxGxxG normally present in the loop between beta strands 1 and 2 but contain 7 of the 12 strictly conserved residues present in most protein kinases, including the HGDxxxN signature sequence in kinase subdomain VIB. Furthermore, APH also has been shown to exhibit protein-serine/threonine kinase activity, suggesting that other YLK-related molecules may indeed be functional protein kinases.

The eukaryotic lipid kinases (PI3Ks, PI4Ks, and PIPKs) also contain several short motifs similar to protein kinases, but otherwise share minimal primary sequence similarity. However, once again structural analysis of PIPKII-beta defines a conserved ATP-binding core that is strikingly similar to conventional protein kinases. Three residues are conserved among all of these enzymes including (relative to the PKA sequence) Lys-72 which binds the gamma-phosphate of ATP, Asp-166 which is part of the HRDLK motif and Asp-184 from the conserved Mg⁺⁺ or Mn⁺⁺ binding DFG motif. The worm genome contains 12 phosphatidylinositol kinases, including 3 PI3-kinases, 2 PI4-kinases, 3 PIP5-kinases, and 4 P13-kinase-related kinases. The latter group has 4 mammalian members (DNA-PK, FRAP/TOR, ATM, and ATR), which have been shown to participate in the maintenance of genomic integrity in response to DNA damage, and exhibit true protein kinase activity, raising the possibility that other PI-kinases may also act as protein kinases. Regardless of whether they have true protein kinase activity, PI3-kinases are tightly linked to protein kinase signaling, as evidenced by their involvement downstream of many growth factor receptors and as upstream activators of the cell survival response mediated by the AKT protein kinase.

Although members of protein kinase subfamilies are different from each other on the primary sequence level and can be involved in different cellular processses, structurally they are very similar: they have the same folding patterns, secondary structure patterns, and structure of the ATP binding pocket; they also utilize the same conserved amino acid residues of the ATP-binding pocket to perform the same biochemical function: transfer a phosphate group of an ATP molecule onto a substrate. These conserved residues embedded into secondary structure pattern (CRISSP) suggests a new way of identifying novel members of this and other protein families.

### SUMMARY OF THE INVENTION

The present invention relates to a method for detecting remote polypeptide homologues, comprising analysis of conserved secondary structure pattern in a protein family, and conserved active site amino acid residues. The analyses are used to identify conserved residues embedded into the secondary structure pattern (CRISSP), which are used to detect remote homologues of the referent protein family. The method can detect remote homologues that cannot be detected using sequence or secondary structure-based methods.

The method includes a method for identifying a remote polypeptide homologue to a referent protein family, comprising:

(a) identifying the conserved secondary structure pattern (CSSP) of said protein family;

(b) identifying the conserved amino acid residues (CAAR) or conserved active site amino acid residues (CASAAR) of the referent protein family; and

(c) identifying the conserved residues embedded into the secondary structure pattern (CRISSP); and

(d) identifying the candidate as a remote homologue if the candidate polypeptide contains the CRISSP of (c).

In other embodiments, the invention includes the remote polypeptide homologue detection method, wherein said referent protein family is the protein kinase family. In other embodiments, the referent protein family is the phosphatase family or the protease family or the nuclear hormone receptor family. As described herein, the secondary structure pattern can be identified using DSSP and the CASAAR can be are identified using FSSP database.

The invention also includes a computer readable medium having program code stored thereon for identifying a remote polypeptide homologue to a referent protein family, the program code configured to cause a computer to perform the following steps:

(a) identifying the conserved secondary structure pattern of said protein family;

(b) identifying the conserved amino acid residues or conserved active site amino acid residues of the referent protein family; and

(c) identifying the conserved residues embedded into the secondary structure pattern (CRISSP); and

(d) identifying the candidate as a remote homologue if the candidate polypeptide contains the CRISSP of (c).

The invention also includes a programmed storage device comprising instructions that when executed perform the steps of:

(a) identifying the conserved secondary structure pattern of said protein family;

(b) identifying the conserved amino acid residues or conserved active site amino acid residues of the referent protein family; and

(c) identifying the conserved residues embedded into the secondary structure pattern (CRISSP); and

(d) identifying the candidate as a remote homologue if the candidate polypeptide contains the CRISSP of (c).

The invention further includes a process for effecting analysis of a polypeptide sequence through use of a computer having a memory, said process comprising:

(a) placing into said memory data representing a polypeptide,

(b) developing within said memory a data structure associated with said data and reflecting the underlying organization and structure of the data to facilitate program access to data elements corresponding to logical sub-components of the sequence,

(c) programming said computer with a program containing instructions sufficient to implement the method of claim 1, and

(d) executing said program on said computer while granting said program access to said data and to said data structure within said memory.

“remote polypeptide homologue” is used to refer to a polypeptide that has negligible amino acid sequence homology compared to a referent polypeptide domain, polypeptide, or polypeptide family, but has function that is substantially the same as that of the referent. The skilled artisan will recognize that sequence homology can be determined using methods using known algorithms, such as the Smith-Waterman algorithm. “Negligible homology” in this context denotes less than about 55%, preferably less than 35%, and more preferably less than about 25% identical amino acid residues between one polypeptide sequence and a referent polypeptide sequence. For example, the catalytic domain of the family of protein kinases has been well-characterized; a remote kinase homologue may share less than 25% sequence homology with the catalytic domain, yet retain kinase activity.

“conserved secondary structure pattern” or “CSSP” is used to refer to secondary structures that are conserved, or maintained, throughout a majority (at least 75%, 80% or 85%, more preferably at least 90%, 91%, 92%, 93%, 94%, 95%, 96%, 97%, 98%, 99%) of the members of a protein family. The pattern of secondary structure is determined by identifying the presence and order of secondary structure elements, such as helix (denoted herein by “h”), beta-strand (denoted herein by “e”) and loop (denoted herein by underscore). The skilled artisan will recognize that secondary structure can be predicted using programs known in the art, such as PSIPRED (Jones, 1999), which analyze primary sequence information. Other programs, such as DSSP (Kabsch & Sander, 1983), also can be used to derive secondary structure patterns.

“conserved amino acid residues” or “CAAR” is used to refer to amino acids that are conserved throughout a majority of the members of a protein family (at least 75%, 80% or 85%, more preferably at least 90%, 91%, 92%, 93%, 94%, 95%, 96%, 97%, 98%, 99%). These conserved residues are identified from structure-based alignments of the amino acid sequences of a group of proteins, for which three-dimensional structures of the proteins are known. The skilled artisan will recognize that protein structure alignments can be obtained using programs known in the art, such as DALI (Holm & Sander, 1993), or from the database FSSP (Holm & Sander, 1996) of such alignments, both showing the alignments in linear/sequence fashion and thus allowing one to identify the conserved residues. Preferably, a structural alignment of at least two remote homologues of known structure is conducted to identify highly conserved amino acid residues.

“conserved active site amino acid residues” or “CASAAR” is used to refer to amino acids that are conserved in an active site throughout a majority (at least 75%, 80% or 85%, more preferably at least 90%, 91%, 92%, 93%, 94%, 95%, 96%, 97%, 98%, 99%) of the members of a protein family. Typically, CASAAR is a subset of CAAR and consists of the conserved residues that are located within the active site(s) of the protein. Accordingly, CASAAR are determined using methodology like that used to determined CAAR.

In this context, “active site” is used to denote one or more regions of a polypeptide that are important for polypeptide function. For example, certain amino acid residues of the catalytic domain of a protein kinase, which has enzymatic activity, interact with ATP molecules and hence form an active site. These residues can be identified from analysis of three-dimensional structures of proteins with bound ligands or analogs as well as from biochemical studies such as mutagenesis. For purposes of this application, an active site can also denote other functionally important amino acid residues, including, but not limited to, those involved in binding ligands, substrates, and regulators.

“conserved residues embedded into the secondary structure pattern” or “CRISSP” is used to denote the residues that are identified by superimposing the conserved active site amino acid residues on the conserved secondary structure pattern. Typically, the CRISSP will contain those CAAR or CASAAR that appear within the CSSP. Additionally, the CRISSP preferably is conserved throughout the referent protein family—preferably throughout at least 75%, 80% or 85%, more preferably at least 90%, 91%, 92%, 93%, 94%, 95%, 96%, 97%, 98%, 99%, of the protein family.

“referent protein family” is used to denote a group of functionally related proteins which serve as the reference point for identifying remote polypeptide homologues according to the invention. For example, a protein family may be all protein kinases, or a selected subset of protein kinases, such as a group of proteins with functionally related catalytic domains. Other exemplary referent protein families include, but are not limited to, proteases, phosphatases, and nuclear hormone receptors. The inventive method is suitable for analysis of other enzymes, as well as other polypeptide families.

The present invention also relates, in part, to human protein kinases and protein kinase-like enzymes identified using the CRISSP methods of the invention.

Tyrosine and serine/threonine kinases (PTK's and STK's) have been identified and their protein sequence predicted as part of the instant invention. Mammalian members of these families were identified through the use of a bioinformatics strategy described herein. The partial or complete sequences of these kinases are presented here, together with their classification, predicted or deduced protein structure.

One aspect of the invention features an identified, isolated, enriched, or purified nucleic acid molecule encoding a kinase polypeptide having an amino acid sequence selected from the group consisting of those set forth in SEQ ID NOS:1-87.

The term “identified” in reference to a nucleic acid is meant that a sequence was selected from a genomic, EST, or cDNA sequence database based on it being predicted to encode a portion of a previously unknown or novel protein kinase.

By “isolated,” in reference to nucleic acid, is meant a polymer of 9, 18, 21, 36, or 90 or more nucleotides conjugated to each other, including DNA and RNA that is isolated from a natural source or that is synthesized as the sense or complementary antisense strand. In certain embodiments of the invention, longer nucleic acids are preferred, for example those of 120, 300, 600, 900, 1200, 1500, or more nucleotides and/or those having at least 50%, 60%, 75%, 80%, 85%, 90%, 91%, 92%, 93%, 94%, 95%, 96%, 97%, 98%, 99% or 100% identity to a sequence selected from the group consisting of those set forth in SEQ ID NO:88-174.

The isolated nucleic acid of the present invention is unique in the sense that it is not found in a pure or separated state in nature. Use of the term “isolated” indicates that a naturally occurring sequence has been removed from its normal cellular (i.e., chromosomal) environment. Thus, the sequence may be in a cell-free solution or placed in a different cellular environment. The term does not imply that the sequence is the only nucleotide chain present, but that it is essentially free (about 90-95% pure at least) of non-nucleotide material naturally associated with it, and thus is distinguished from isolated chromosomes.

By the use of the term “enriched” in reference to nucleic acid is meant that the specific DNA or RNA sequence constitutes a significantly higher fraction (2- to 5-fold) of the total DNA or RNA present in the cells or solution of interest than in normal or diseased cells or in the cells from which the sequence was taken. This could be caused by a person by preferential reduction in the amount of other DNA or RNA present, or by a preferential increase in the amount of the specific DNA or RNA sequence, or by a combination of the two. However, it should be noted that enriched does not imply that there are no other DNA or RNA sequences present, just that the relative amount of the sequence of interest has been significantly increased. The term “significant” is used to indicate that the level of increase is useful to the person making such an increase, and generally means an increase relative to other nucleic acids of about at least 2-fold, more preferably at least 5- to 10-fold or even more. The term also does not imply that there is no DNA or RNA from other sources. The DNA from other sources may, for example, comprise DNA from a yeast or bacterial genome, or a cloning vector such as pUC19. This term distinguishes from naturally occurring events, such as viral infection, or tumor-type growths, in which the level of one mRNA may be naturally increased relative to other species of mRNA. That is, the term is meant to cover only those situations in which a person has intervened to elevate the proportion of the desired nucleic acid.

It is also advantageous for some purposes that a nucleotide sequence be in purified form. The term “purified” in reference to nucleic acid does not require absolute purity (such as a homogeneous preparation). Instead, it represents an indication that the sequence is relatively more pure than in the natural environment (compared to the natural level this level should be at least 2- to 5-fold greater, e.g., in terms of mg/mL). Individual clones isolated from a cDNA library may be purified to electrophoretic homogeneity. The claimed DNA molecules obtained from these clones could be obtained directly from total DNA or from total RNA. The cDNA clones are not naturally occurring, but rather are preferably obtained via manipulation of a partially purified naturally occurring substance (messenger RNA). The construction of a cDNA library from mRNA involves the creation of a synthetic substance (cDNA) and pure individual cDNA clones can be isolated from the synthetic library by clonal selection of the cells carrying the cDNA library. Thus, the process which includes the construction of a cDNA library from mRNA and isolation of distinct cDNA clones yields an approximately 10⁶-fold purification of the native message. Thus, purification of at least one order of magnitude, preferably two or three orders, and more preferably four or five orders of magnitude is expressly contemplated.

By a “kinase polypeptide” is meant 20, 25, 32 (preferably 40, more preferably 45, most preferably 55) or more contiguous amino acids in a polypeptide having an amino acid sequence selected from the group consisting of those set forth in SEQ ID NO:1-87. In certain aspects, polypeptides of 100, 200, 300, 400, 450, 500, 550, 600, 700, 800, 900 or more amino acids are preferred. The kinase polypeptide can be encoded by a full-length nucleic acid sequence or any portion (e.g., a “fragment” as defined herein) of the full-length nucleic acid sequence, so long as a functional activity of the polypeptide is retained, including, for example, a catalytic domain, as defined herein, or a portion thereof. One of skill in the art would be able to select those catalytic domains, or portions thereof, which exhibit a kinase or kinase-like activity, e.g., catalytic activity, as defined herein. It is well known in the art that due to the degeneracy of the genetic code numerous different nucleic acid sequences can code for the same amino acid sequence. Equally, it is also well known in the art that conservative changes in amino acid sequence can be made to arrive at a protein or polypeptide which retains the functionality of the original. Such substitutions may include the replacement of an amino acid by a residue having similar physicochemical properties, such as substituting one aliphatic residue (Ile, Val, Leu or Ala) for another, or substitution between basic residues Lys and Arg, acidic residues Giu and Asp, amide residues Gln and Asn, hydroxyl residues Ser and Tyr, or aromatic residues Phe and Tyr. Further information regarding making amino acid exchanges which have only slight, if any, effects on the overall protein can be found in Bowie et al., Science, 1990, 247, 1306-1310, which is incorporated herein by reference in its entirety including any figures, tables, or drawings. In all cases, all permutations are intended to be covered by this disclosure.

The amino acid sequence of a kinase peptide of the invention will be substantially similar to a sequence having an amino acid sequence selected from the group consisting of those set forth in SEQ ID NO:1-87, or the corresponding full-length amino acid sequence, or fragments thereof.

A sequence that is substantially similar to a sequence selected from the group consisting of those set forth in SEQ ID NO:1-87, will preferably have at least 80, 85%, 90% identity (more preferably at least 91%, 92%, 93%, 94%, 95%, 96%, 97%, 98%, 99% 100%) to the sequence.

By “identity” is meant a property of sequences that measures their similarity or relationship. Identity is measured by dividing the number of identical residues by the total number of residues and gaps and multiplying the product by 100. “Gaps” are spaces in an alignment that are the result of additions or deletions of amino acids. Thus, two copies of exactly the same sequence have 100% identity, but sequences that are less highly conserved, and have deletions, additions, or replacements, may have a lower degree of identity. Those skilled in the art will recognize that several computer programs are available for determining sequence identity using standard parameters, for example Gapped BLAST or PSI-BLAST (Altschul, et al. (1997) Nucleic Acids Res. 25:3389-3402), BLAST (Altschul, et al. (1990) J. Mol. Biol. 215:403-410), and Smith-Waterman (Smith, et al. (1981) J. Mol. Biol. 147:195-197). Preferably, the default settings of these programs will be employed, but those skilled in the art recognize whether these settings need to be changed and know how to make the changes.

“Similarity” is measured by dividing the number of identical residues plus the number of conservatively substituted residues (see Bowie, et al. Science, 1999), 247, 1306-1310, which is incorporated herein by reference in its entirety, including any drawings, figures, or tables) by the total number of residues and gaps and multiplying the product by 100.

In preferred embodiments, the invention features isolated, enriched, or purified nucleic acid molecules encoding a kinase polypeptide comprising a nucleotide sequence that: (a) encodes a polypeptide having an amino acid sequence selected from the group consisting of those set forth in SEQ ID NO: 1-87; (b) is the complement of the nucleotide sequence of (a); (c) hybridizes under highly stringent conditions to the nucleotide molecule of (a) and encodes a naturally occurring kinase polypeptide; (d) encodes a polypeptide having an amino acid sequence selected from the group consisting of those set forth in SEQ ID NO:1-87, except that it lacks one or more, but not all, of the domains selected from the group consisting of an N-terminal domain, a catalytic domain, a C-terminal catalytic domain, a C-terminal domain, a coiled-coil structure region, a proline-rich region, a spacer region, and a C-terminal tail; and (e) is the complement of the nucleotide sequence of (d).

The term “complement” refers to two nucleotides that can form multiple favorable interactions with one another. For example, adenine is complementary to thymine as they can form two hydrogen bonds. Similarly, guanine and cytosine are complementary since they can form three hydrogen bonds. A nucleotide sequence is the complement of another nucleotide sequence if all of the nucleotides of the first sequence are complementary to all of the nucleotides of the second sequence. The invention includes complements of SEQ ID NOS:88-174.

Various low or high stringency hybridization conditions may be used depending upon the specificity and selectivity desired. These conditions are well known to those skilled in the art. Under stringent hybridization conditions only highly complementary nucleic acid sequences hybridize. Preferably, such conditions prevent hybridization of nucleic acids having more than 1 or 2 mismatches out of 20 contiguous nucleotides, more preferably, such conditions prevent hybridization of nucleic acids having more than 1 or 2 mismatches out of 50 contiguous nucleotides, most preferably, such conditions prevent hybridization of nucleic acids having more than 1 or 2 mismatches out of 100 contiguous nucleotides. In some instances, the conditions may prevent hybridization of nucleic acids having more than 5 mismatches in the full-length sequence.

By stringent hybridization assay conditions is meant hybridization assay conditions at least as stringent as the following: hybridization in 50% formamide, 5×SSC, 50 mM NaH2PO4, pH 6.8, 0.5% SDS, 0.1 mg/mL sonicated salmon sperm DNA, and 5× Denhardt's solution at 42° C. overnight; washing with 2×SSC, 0.1% SDS at 45° C.; and washing with 0.2×SSC, 0.1% SDS at 45° C. Under some of the most stringent hybridization assay conditions, the second wash can be done with 0.1×SSC at a temperature up to 70° C. (Berger et al. (1987) Guide to Molecular Cloning Techniques pg 421, hereby incorporated by reference herein in its entirety including any figures, tables, or drawings.). However, other applications may require the use of conditions falling between these sets of conditions. Methods of determining the conditions required to achieve desired hybridizations are well known to those with ordinary skill in the art, and are based on several factors, including but not limited to, the sequences to be hybridized and the samples to be tested. Washing conditions of lower stringency frequently utilize a lower temperature during the washing steps, such as 65° C., 60° C., 55° C., 50° C., or 42° C.

The term “domain” refers to a region of a polypeptide which serves a particular function. For instance, N-terminal or C-terminal domains of signal transduction proteins can serve functions including, but not limited to, binding molecules that localize the signal transduction molecule to different regions of the cell or binding other signaling molecules directly responsible for propagating a particular cellular signal. Some domains can be expressed separately from the rest of the protein and function by themselves, while others must remain part of the intact protein to retain function. The latter are termed functional regions of proteins and also relate to domains.

The term “N-terminal domain” refers to the extracatalytic region located between the initiator methionine and the catalytic domain of the protein kinase. The N-terminal domain can be identified following a Smith-Waterman alignment of the protein sequence against the non-redundant protein database to define the N-terminal boundary of the catalytic domain. Depending on its length, the N-terminal domain may or may not play a regulatory role in kinase function. An example of a protein kinase whose N-terminal domain has been shown to play a regulatory role is PAK5, which contains a CRIB motif used for Cdc42 and rac binding (Burbelo, P. D. et al. (1995) J. Biol. Chem. 270, 29071-29074).

The term “catalytic domain” refers to a region of the protein kinase that is typically 25-300 amino acids long and is responsible for carrying out the phosphate transfer reaction from a high-energy phosphate donor molecule such as ATP or GTP to itself (autophosphorylation) or to other proteins (exogenous phosphorylation). The catalytic domain of protein kinases is made up of 12 subdomains that contain highly conserved amino acid residues, and are responsible for proper polypeptide folding and for catalysis. The catalytic domain can be identified following a Smith-Waterman alignment of the protein sequence against the non-redundant protein database.

The term “catalytic activity”, as used herein, defines the rate at which a kinase catalytic domain phosphorylates a substrate. Catalytic activity can be measured, for example, by determining the amount of a substrate converted to a phosphorylated product as a function of time. Catalytic activity can be measured by methods of the invention by holding time constant and determining the concentration of a phosphorylated substrate after a fixed period of time. Phosphorylation of a substrate occurs at the active site of a protein kinase. The active site is normally a cavity in which the substrate binds to the protein kinase and is phosphorylated.

The term “substrate” as used herein refers to a molecule phosphorylated by a kinase of the invention. Kinases phosphorylate substrates on serine/threonine or tyrosine amino acids. The molecule may be another protein or a polypeptide.

The term “C-terminal domain” refers to the region located between the catalytic domain or the last (located closest to the C-terminus) functional domain and the carboxy-terminal amino acid residue of the protein kinase. By “functional” domain is meant any region of the polypeptide that may play a regulatory or catalytic role as predicted from amino acid sequence homology to other proteins or by the presence of amino acid sequences that may give rise to specific structural conformations (e.g. N-terminal domain). The C-terminal domain can be identified by using a Smith-Waterman alignment of the protein sequence against the non-redundant protein database to define the C-terminal boundary of the catalytic domain or of any functional C-terminal extracatalytic domain. Depending on its length and amino acid composition, the C-terminal domain may or may not play a regulatory role in kinase function. An example of a protein kinase whose C-terminal domain may play a regulatory role is PAK3 which contains a heterotrimeric G_{b} subunit-binding site near its C-terminus (Leeuw, T. et al. (1998) Nature, 391, 191-195). For the some of the kinases of the instant invention, the C-terminal domain may also comprise the catalytic domain (above).

The term “C-terminal tail” as used herein, refers to a C-terminal domain of a protein kinase, that by homology extends or protrudes past the C-terminal amino acid of its closest homolog. C-terminal tails can be identified by using a Smith-Waterman sequence alignment of the protein sequence against the non-redundant protein database, or by means of a multiple sequence alignment of homologous sequences using the DNAStar program Megalign. Depending on its length, a C-terminal tail may or may not play a regulatory role in kinase function.

The term “coiled-coil structure region” as used herein, refers to a polypeptide sequence that has a high probability of adopting a coiled-coil structure as predicted by computer algorithms such as COILS (Lupas, A. (1996) Meth. Enzymology 266:513-525). Coiled-coils are formed by two or three amphipathic α-helices in parallel. Coiled-coils can bind to coiled-coil domains of other polypeptides resulting in homo- or heterodimers (Lupas, A. (1991) Science 252:1162-1164). Coiled-coil-dependent oligomerization has been shown to be necessary for protein function including catalytic activity of serine/threonine kinases (Roe, J. et al. (1997) J. Biol. Chem. 272:5838-5845).

The term “proline-rich region” as used herein, refers to a region of a protein kinase whose proline content over a given amino acid length is higher than the average content of this amino acid found in proteins (i.e., >10%). Proline-rich regions are easily discernable by visual inspection of amino acid sequences and quantitated by standard computer sequence analysis programs such as the DNAStar program EditSeq. Proline-rich regions have been demonstrated to participate in regulatory protein-protein interactions. Among these interactions, those that are most relevant to this invention involve the “PxxP” proline rich motif found in certain protein kinases (i.e., human PAK1) and the SH3 domain of the adaptor molecule Nck (Galisteo, M. L. et al. (1996) J. Biol. Chem. 271:20997-21000). Other regulatory interactions involving “PxxP” proline-rich motifs include the WW domain (Sudol, M. (1996) Prog. Biochys. Mol. Bio. 65:113-132).

The term “spacer region” as used herein, refers to a region of the protein kinase located between predicted functional domains. The spacer region has no detectable homology to any amino acid sequence in the database, and can be identified by using a Smith-Waterman alignment of the protein sequence against the non-redundant protein database to define the C- and N-terminal boundaries of the flanking functional domains. Spacer regions may or may not play a fundamental role in protein kinase function. Precedence for the regulatory role of spacer regions in kinase function is provided by the role of the src kinase spacer in inter-domain interactions (Xu, W. et al. (1997) Nature 385:595-602).

The term “insert” as used herein refers to a portion of a protein kinase that is absent from a close homolog. Inserts may or may not by the product alternative splicing of exons. Inserts can be identified by using a Smith-Waterman sequence alignment of the protein sequence against the non-redundant protein database, or by means of a multiple sequence alignment of homologous sequences using the DNAStar program Megalign. Inserts may play a functional role by presenting a new interface for protein-protein interactions, or by interfering with such interactions.

The term “signal transduction pathway” refers to the molecules that propagate an extracellular signal through the cell membrane to become an intracellular signal. This signal can then stimulate a cellular response. The polypeptide molecules involved in signal transduction processes are typically receptor and non-receptor protein tyrosine kinases, receptor and non-receptor protein phosphatases, polypeptides containing SRC homology 2 and 3 domains, phosphotyrosine binding proteins (SRC homology 2 (SH2) and phosphotyrosine binding (PTB and PH) domain containing proteins), proline-rich binding proteins (SH3 domain containing proteins), GTPases, phosphodiesterases, phospholipases, prolyl isomerases, proteases, Ca2+ binding proteins, cAMP binding proteins, guanyl cyclases, adenylyl cyclases, NO generating proteins, nucleotide exchange factors, and transcription factors.

In other preferred embodiments, the invention features isolated, enriched, or purified nucleic acid molecules encoding kinase polypeptides, further comprising a vector or promoter effective to initiate transcription in a host cell.

The invention also features recombinant nucleic acid, preferably in a cell or an organism. The recombinant nucleic acid may contain a sequence selected from the group consisting of those set forth in SEQ ID NO: 88-175, or a functional derivative thereof and a vector or a promoter effective to initiate transcription in a host cell. The recombinant nucleic acid can alternatively contain a transcriptional initiation region functional in a cell, a sequence complementary to an RNA sequence encoding a kinase polypeptide and a transcriptional termination region functional in a cell. Specific vectors and host cell combinations are discussed herein.

The term “vector” relates to a single or double-stranded circular nucleic acid molecule that can be transfected into cells and replicated within or independently of a cell genome. A circular double-stranded nucleic acid molecule can be cut and thereby linearized upon treatment with restriction enzymes. An assortment of nucleic acid vectors, restriction enzymes, and the knowledge of the nucleotide sequences cut by restriction enzymes are readily available to those skilled in the art. A nucleic acid molecule encoding a kinase can be inserted into a vector by cutting the vector with restriction enzymes and ligating the two pieces together.

The term “transfecting” defines a number of methods to insert a nucleic acid vector or other nucleic acid molecules into a cellular organism. These methods involve a variety of techniques, such as treating the cells with high concentrations of salt, an electric field, detergent, or DMSO to render the outer membrane or wall of the cells permeable to nucleic acid molecules of interest or use of various viral transduction strategies.

The term “promoter” as used herein, refers to nucleic acid sequence needed for gene sequence expression. Promoter regions vary from organism to organism, but are well known to persons skilled in the art for different organisms. For example, in prokaryotes, the promoter region contains both the promoter (which directs the initiation of RNA transcription) as well as the DNA sequences which, when transcribed into RNA, will signal synthesis initiation. Such regions will normally include those 5′-non-coding sequences involved with initiation of transcription and translation, such as the TATA box, capping sequence, CAAT sequence, and the like.

In preferred embodiments, the isolated nucleic acid comprises, consists essentially of, or consists of a nucleic acid sequence selected from the group consisting of those set forth in SEQ ID NO:88-174, or which encodes an amino acid sequence selected from the group consisting of those set forth in SEQ ID NO:1-87, a functional derivative thereof, or at least 35, 40, 45, 50, 60, 75, 100, 200, or 300 contiguous amino acids selected from the group consisting of those set forth in SEQ ID NO:1-87, or at least 100, 200, 300 or 400 contiguous nucleotides selected from the group consisting of those set forth in SEQ ID NO:88-174. The nucleic acid may be isolated from a natural source by cDNA cloning or by subtractive hybridization. The natural source may be mammalian, preferably human, preferably blood, semen or tissue, and the nucleic acid may be synthesized by the triester method or by using an automated DNA synthesizer.

The term “mammal” refers preferably to such organisms as mice, rats, rabbits, guinea pigs, sheep, and goats, more preferably to cats, dogs, monkeys, and apes, and most preferably to humans.

In yet other preferred embodiments, the nucleic acid is a conserved or unique region, for example those useful for: the design of hybridization probes to facilitate identification and cloning of additional polypeptides, the design of PCR probes to facilitate cloning of additional polypeptides, obtaining antibodies to polypeptide regions, and designing antisense oligonucleotides.

By “conserved nucleic acid regions”, are meant regions present on two or more nucleic acids encoding a kinase polypeptide, to which a particular nucleic acid sequence can hybridize under lower stringency conditions. Examples of lower stringency conditions suitable for screening for nucleic acid encoding kinase polypeptides are provided in Wahl et al. Meth. Enzym. 152:399-407 (1987) and in Wahl et al. Meth. Enzym. 152:415-423 (1987), which are hereby incorporated by reference herein in its entirety, including any drawings, figures, or tables. Preferably, conserved regions differ by no more than 5 out of 20 nucleotides, even more preferably 2 out of 20 nucleotides or most preferably 1 out of 20 nucleotides.

By “unique nucleic acid region” is meant a sequence present in a nucleic acid coding for a kinase polypeptide that is not present in a sequence coding for any other naturally occurring polypeptide. Such regions preferably encode 32 (preferably 40, more preferably 45, most preferably 55) or more contiguous amino acids, for example, an amino acid sequence selected from the group consisting of those set forth in SEQ ID NO:1-87. In particular, a unique nucleic acid region is preferably of mammalian origin.

Another aspect of the invention features a nucleic acid probe for the detection of nucleic acid encoding a kinase polypeptide having an amino acid sequence selected from the group consisting of those set forth in SEQ ID NO:1-87 in a sample. The nucleic acid probe contains a nucleotide base sequence that will hybridize to the sequence selected from the group consisting of those set forth in SEQ ID NO:88-174, or a functional derivative thereof.

In preferred embodiments, the nucleic acid probe is at least 12, 18, 25, 32, 75, 90, 100, 120, 150, 200, 250, 300 or 350 contiguous nucleic acids, wherein the nucleic acid sequence is selected from the group consisting of SEQ ID NO:88-174, or a functional derivative thereof. More preferably, the probe is at least 9, 18, 21, 32, 75 or 90 nucleotides.

Methods for using the probes include detecting the presence or amount of kinase RNA in a sample by contacting the sample with a nucleic acid probe under conditions such that hybridization occurs and detecting the presence or amount of the probe bound to kinase RNA. The nucleic acid duplex formed between the probe and a nucleic acid sequence coding for a kinase polypeptide may be used in the identification of the sequence of the nucleic acid detected (Nelson et al., in Nonisotopic DNA Probe Techniques, Academic Press, San Diego, Kricka, ed., p. 275, 1992, hereby incorporated by reference herein in its entirety, including any drawings, figures, or tables). Kits for performing such methods may be constructed to include a container means having disposed therein a nucleic acid probe.

Methods for using the probes also include using these probes to find, for example, the full-length clone of each of the predicted kinases by techniques known to one skilled in the art. These clones will be useful for screening for small molecule compounds that inhibit the catalytic activity of the encoded kinase with potential utility in treating cancers, immune-related diseases and disorders, cardiovascular disease, brain or neuronal-associated diseases, and metabolic disorders. More specifically disorders including cancers of tissues or blood, or hematopoietic origin, particularly those involving breast., colon, lung, prostate, cervical, brain, ovarian, bladder, or kidney; central or peripheral nervous system diseases and conditions including migraine, pain, sexual dysfunction, mood disorders, attention disorders, cognition disorders, hypotension, and hypertension; psychotic and neurological disorders, including anxiety, schizophrenia, manic depression, delirium, dementia, severe mental retardation and dyskinesias, such as Huntington's disease or Tourette's Syndrome; neurodegenerative diseases including Alzheimer's, Parkinson's, multiple sclerosis, and amyotrophic lateral sclerosis; viral or non-viral infections caused by HIV-1, HIV-2 or other viral- or prion-agents or fungal- or bacterial-organisms; metabolic disorders including Diabetes and obesity and their related syndromes, among others; cardiovascular disorders including reperfusion restenosis, coronary thrombosis, clotting disorders, unregulated cell growth disorders, atherosclerosis; ocular disease including glaucoma, retinopathy, and macular degeneration; inflammatory disorders including rheumatoid arthritis, chronic inflammatory bowel disease, chronic inflammatory pelvic disease, multiple sclerosis, asthma, osteoarthritis, psoriasis, atherosclerosis, rhinitis, autoimmunity, and organ transplant rejection.

In another aspect, the invention describes a recombinant cell or tissue comprising a nucleic acid molecule encoding a kinase polypeptide having an amino acid sequence selected from the group consisting of those set forth in SEQ ID NO:1-87. In such cells, the nucleic acid may be under the control of the genomic regulatory elements, or may be under the control of exogenous regulatory elements including an exogenous promoter. By “exogenous” it is meant a promoter that is not normally coupled in vivo transcriptionally to the coding sequence for the kinase polypeptides.

The polypeptide is preferably a fragment of the protein encoded by an amino acid sequence selected from the group consisting of those set forth in SEQ ID NO:1-87. By “fragment,” is meant an amino acid sequence present in a kinase polypeptide. Preferably, such a sequence comprises at least 32, 45, 50, 60, 100, 200, or 300 contiguous amino acids of a sequence selected from the group consisting of those set forth in SEQ ID NO:1-87.

In another aspect, the invention features an isolated, enriched, or purified kinase polypeptide having the amino acid sequence selected from the group consisting of those set forth in SEQ ID NO:1-87.

By “isolated” in reference to a polypeptide is meant a polymer of 6 (preferably 12, more preferably 18, most preferably 25, 32, 40, or 50) or more amino acids conjugated to each other, including polypeptides that are isolated from a natural source or that are synthesized. In certain aspects longer polypeptides are preferred, such as those comprising 100, 200, 300, 400, 450, 500, 550, 600, 700, 800, 900 or more contiguous amino acids, including an amino acid sequence selected from the group consisting of those set forth in SEQ ID NO:1-87.

The isolated polypeptides of the present invention are unique in the sense that they are not found in a pure or separated state in nature. Use of the term “isolated” indicates that a naturally occurring sequence has been removed from its normal cellular environment. Thus, the sequence may be in a cell-free solution or placed in a different cellular environment. The term does not imply that the sequence is the only amino acid chain present, but that it is essentially free (about 90-95% pure at least) of non-amino acid-based material naturally associated with it.

By the use of the term “enriched” in reference to a polypeptide is meant that the specific amino acid sequence constitutes a significantly higher fraction (2- to 5-fold) of the total amino acid sequences present in the cells or solution of interest than in normal or diseased cells or in the cells from which the sequence was taken. This could be caused by a person by preferential reduction in the amount of other amino acid sequences present, or by a preferential increase in the amount of the specific amino acid sequence of interest, or by a combination of the two. However, it should be noted that enriched does not imply that there are no other amino acid sequences present, just that the relative amount of the sequence of interest has been significantly increased. The term “significantly” here is used to indicate that the level of increase is useful to the person making such an increase, and generally means an increase relative to other amino acid sequences of about at least 2-fold, more preferably at least 5- to 10-fold or even more. The term also does not imply that there is no amino acid sequence from other sources. The other source of amino acid sequences may, for example, comprise amino acid sequence encoded by a yeast or bacterial genome, or a cloning vector such as pUC19. The term is meant to cover only those situations in which man has intervened to increase the proportion of the desired amino acid sequence.

It is also advantageous for some purposes that an amino acid sequence be in purified form. The term “purified” in reference to a polypeptide does not require absolute purity (such as a homogeneous preparation); instead, it represents an indication that the sequence is relatively purer than in the natural environment. Compared to the natural level this level should be at least 2-to 5-fold greater (e.g., in terms of mg/mL). Purification of at least one order of magnitude, preferably two or three orders, and more preferably four or five orders of magnitude is expressly contemplated. The substance is preferably free of contamination at a functionally significant level, for example 90%, 95%, or 99% pure.

In preferred embodiments, the kinase polypeptide comprises an amino acid sequence having (a) an amino acid sequence selected from the group consisting of those set forth in SEQ ID NO:1-87; and (b) an amino acid sequence selected from the group consisting of those set forth in SEQ ID NO:1-87, except that it lacks one or more of the domains selected from the group consisting of a C-terminal catalytic domain, an N-terminal domain, a catalytic domain, a C-terminal domain, a coiled-coil structure region, a proline-rich region, a spacer region, and a C-terminal tail.

The polypeptide can be isolated from a natural source by methods well-known in the art. The natural source may be mammalian, preferably human, preferably blood, semen or tissue, and the polypeptide may be synthesized using an automated polypeptide synthesizer.

In some embodiments the invention includes a recombinant kinase polypeptide having (a) an amino acid sequence selected from the group consisting of those set forth in SEQ ID NO:1-87. By “recombinant kinase polypeptide” is meant a polypeptide produced by recombinant DNA techniques such that it is distinct from a naturally occurring polypeptide either in its location (e.g., present in a different cell or tissue than found in nature), purity or structure. Generally, such a recombinant polypeptide will be present in a cell in an amount different from that normally observed in nature.

The polypeptides to be expressed in host cells may also be fusion proteins which include regions from heterologous proteins. Such regions may be included to allow, e.g., secretion, improved stability, or facilitated purification of the polypeptide. For example, a sequence encoding an appropriate signal peptide can be incorporated into expression vectors. A DNA sequence for a signal peptide (secretory leader) may be fused in-frame to the polynucleotide sequence so that the polypeptide is translated as a fusion protein comprising the signal peptide. A signal peptide that is functional in the intended host cell promotes extracellular secretion of the polypeptide. Preferably, the signal sequence will be cleaved from the polypeptide upon secretion of the polypeptide from the cell. Thus, preferred fusion proteins can be produced in which the N-terminus of a kinase polypeptide is fused to a carrier peptide.

In one embodiment, the polypeptide comprises a fusion protein which includes a heterologous region used to facilitate purification of the polypeptide. Many of the available peptides used for such a function allow selective binding of the fusion protein to a binding partner. A preferred binding partner includes one or more of the TgG binding domains of protein A are easily purified to homogeneity by affinity chromatography on, for example, IgG-coupled Sepharose. Alternatively, many vectors have the advantage of carrying a stretch of histidine residues that can be expressed at the N-terminal or C-terminal end of the target protein, and thus the protein of interest can be recovered by metal chelation chromatography. A nucleotide sequence encoding a recognition site for a proteolytic enzyme such as enterokinase, factor X procollagenase or thrombine may immediately precede the sequence for a kinase polypeptide to permit cleavage of the fusion protein to obtain the mature kinase polypeptide. Additional examples of fusion-protein binding partners include, but are not limited to, the yeast I-factor, the honeybee melatin leader in sf9 insect cells, 6-His tag, thioredoxin tag, hemaglutinin tag, GST tag, and OmpA signal sequence tag. As will be understood by one of skill in the art, the binding partner which recognizes and binds to the peptide may be any ion, molecule or compound including metal ions (e.g., metal affinity columns), antibodies, or fragments thereof, and any protein or peptide which binds the peptide, such as the FLAG tag.

In another aspect, the invention features an antibody (e.g., a monoclonal or polyclonal antibody) having specific binding affinity to a kinase polypeptide or a kinase polypeptide domain or fragment where the polypeptide is selected from the group having a sequence at least about 90% identical to an amino acid sequence selected from the group consisting of those set forth in SEQ ID NO:1-87. By “specific binding affinity” is meant that the antibody binds to the target kinase polypeptide with greater affinity than it binds to other polypeptides under specified conditions. Antibodies or antibody fragments are polypeptides that contain regions that can bind other polypeptides. Antibodies can be used to identify an endogenous source of kinase polypeptides, to monitor cell cycle regulation, and for immuno-localization of kinase polypeptides within the cell.

The term “polyclonal” refers to antibodies that are heterogenous populations of antibody molecules derived from the sera of animals immunized with an antigen or an antigenic functional derivative thereof. For the production of polyclonal antibodies, various host animals may be immunized by injection with the antigen. Various adjuvants may be used to increase the immunological response, depending on the host species.

“Monoclonal antibodies” are substantially homogenous populations of antibodies to a particular antigen. They may be obtained by any technique which provides for the production of antibody molecules by continuous cell lines in culture. Monoclonal antibodies may be obtained by methods known to those skilled in the art (Kohler et al., Nature 256:495-497, 1975, and U.S. Pat. No. 4,376,110, both of which are hereby incorporated by reference herein in their entirety including any figures, tables, or drawings).

An antibody of the present invention includes “humanized” monoclonal and polyclonal antibodies. Humanized antibodies are recombinant proteins in which non-human (typically murine) complementarity determining regions of an antibody have been transferred from heavy and light variable chains of the non-human (e.g. murine) immunoglobulin into a human variable domain, followed by the replacement of some human residues in the framework regions of their murine counterparts. Humanized antibodies in accordance with this invention are suitable for use in therapeutic methods. General techniques for cloning murine immunoglobulin variable domains are described, for example, by the publication of Orlandi et al., Proc. Nat'l Acad. Sci. USA 86: 3833 (1989). Techniques for producing humanized monoclonal antibodies are described, for example, by Jones et al., Nature 321:522 (1986), Riechmann et al., Nature 332:323 (1988), Verhoeyen et al., Science 239:1534 (1988), Carter et al., Proc. Nat'l Acad. Sci. USA 89:4285 (1992), Sandhu, Crit. Rev. Biotech. 12:437 (1992), and Singer et al., J. Immun. 150:2844 (1993).

The term “antibody fragment” refers to a portion of an antibody, often the hypervariable region and portions of the surrounding heavy and light chains, that displays specific binding affinity for a particular molecule. A hypervariable region is a portion of an antibody that physically binds to the polypeptide target.

An antibody fragment of the present invention includes a “single-chain antibody,” a phrase used in this description to denote a linear polypeptide that binds antigen with specificity and that comprises variable or hypervariable regions from the heavy and light chains of an antibody. Such single chain antibodies can be produced by conventional methodology. The Vh and Vl regions of the Fv fragment can be covalently joined and stabilized by the insertion of a disulfide bond. See Glockshuber, et al., Biochemistry 1362 (1990). Alternatively, the Vh and Vl regions can be joined by the insertion of a peptide linker. A gene encoding the Vh, Vl and peptide linker sequences can be constructed and expressed using a recombinant expression vector. See Colcher, et al., J. Nat'l Cancer Inst. 82: 1191 (1990). Amino acid sequences comprising hypervariable regions from the Vh and Vl antibody chains can also be constructed using disulfide bonds or peptide linkers.

Antibodies or antibody fragments having specific binding affinity to a kinase polypeptide of the invention may be used in methods for detecting the presence and/or amount of kinase polypeptide in a sample by probing the sample with the antibody under conditions suitable for kinase-antibody immunocomplex formation and detecting the presence and/or amount of the antibody conjugated to the kinase polypeptide. Diagnostic kits for performing such methods may be constructed to include antibodies or antibody fragments specific for the kinase as well as a conjugate of a binding partner of the antibodies or the antibodies themselves.

An antibody or antibody fragment with specific binding affinity to a kinase polypeptide of the invention can be isolated, enriched, or purified from a prokaryotic or eukaryotic organism. Routine methods known to those skilled in the art enable production of antibodies or antibody fragments, in both prokaryotic and eukaryotic organisms. Purification, enrichment, and isolation of antibodies, which are polypeptide molecules, are described above.

Antibodies having specific binding affinity to a kinase polypeptide of the invention may be used in methods for detecting the presence and/or amount of kinase polypeptide in a sample by contacting the sample with the antibody under conditions such that an immunocomplex forms and detecting the presence and/or amount of the antibody conjugated to the kinase polypeptide. Diagnostic kits for performing such methods may be constructed to include a first container containing the antibody and a second container having a conjugate of a binding partner of the antibody and a label, such as, for example, a radioisotope. The diagnostic kit may also include notification of an FDA approved use and instructions therefor.

In another aspect, the invention features a hybridoma which produces an antibody having specific binding affinity to a kinase polypeptide or a kinase polypeptide domain, where the polypeptide is selected from the group having an amino acid sequence selected from the group consisting of those set forth in SEQ ID NO:1-87 or a fragment thereof. By “hybridoma” is meant an immortalized cell line that is capable of secreting an antibody, for example an antibody to a kinase of the invention. In preferred embodiments, the antibody to the kinase comprises a sequence of amino acids that is able to specifically bind a kinase polypeptide of the invention.

In another aspect, the present invention is also directed to kits comprising antibodies that bind to a polypeptide encoded by any of the nucleic acid molecules described above, and a negative control antibody.

The term “negative control antibody” refers to an antibody derived from similar source as the antibody having specific binding affinity, but where it displays no binding affinity to a polypeptide of the invention.

In another aspect, the invention features a kinase polypeptide binding agent able to bind to a kinase polypeptide selected from the group having (a) an amino acid sequence selected from the group consisting of those set forth in SEQ ID NO:1-87 or a fragment thereof. The binding agent is preferably a purified antibody that recognizes an epitope present on a kinase polypeptide of the invention. Other binding agents include molecules that bind to kinase polypeptides and analogous molecules that bind to a kinase polypeptide. Such binding agents may be identified by using assays that measure kinase binding partner activity, such as those that measure PDGFR activity.

The invention also features a method for screening for human cells containing a kinase polypeptide of the invention or an equivalent sequence. The method involves identifying the novel polypeptide in human cells using techniques that are routine and standard in the art, such as those described herein for identifying the kinases of the invention (e.g., cloning, Southern or Northern blot analysis, in situ hybridization, PCR amplification, etc.).

In another aspect, the invention features methods for identifying a substance that modulates kinase activity comprising the steps of: (a) contacting a kinase polypeptide selected from the group having an amino acid sequence selected from the group consisting of those set forth in SEQ ID NO:1-87 or the catalytic region thereof with a test substance; (b) measuring the activity of said polypeptide; and (c) determining whether said substance modulates the activity of said polypeptide. The skilled artisan will appreciate that the kinase polypeptides of the invention, including, for example, a portion of a full-length sequence such as a catalytic domain or a portion thereof, are useful for the identification of a substance which modulates kinase activity. Those kinase polypeptides having a functional activity (e.g., catalytic activity as defined herein) are useful for identifying a substance that modulates kinase activity.

The term “modulates” refers to the ability of a compound to alter the function of a kinase of the invention. A modulator preferably activates or inhibits the activity of a kinase of the invention depending on the concentration of the compound exposed to the kinase.

The term “modulates” also refers to altering the function of kinases of the invention by increasing or decreasing the probability that a complex forms between the kinase and a natural binding partner. A modulator preferably increases the probability that such a complex forms between the kinase and the natural binding partner, more preferably increases or decreases the probability that a complex forms between the kinase and the natural binding partner depending on the concentration of the compound exposed to the kinase, and most preferably decreases the probability that a complex forms between the kinase and the natural binding partner.

The term “activates” refers to increasing the cellular activity of the kinase. The term inhibit refers to decreasing the cellular activity of the kinase. Kinase activity is preferably the interaction with a natural binding partner.

The term “complex” refers to an assembly of at least two molecules bound to one another. Signal transduction complexes often contain at least two protein molecules bound to one another. For instance, a protein tyrosine receptor protein kinase, GRB2, SOS, RAF, and RAS assemble to form a signal transduction complex in response to a mitogenic ligand.

The term “natural binding partner” refers to polypeptides, lipids, small molecules, or nucleic acids that bind to kinases in cells. A change in the interaction between a kinase and a natural binding partner can manifest itself as an increased or decreased probability that the interaction forms, or an increased or decreased concentration of kinase/natural binding partner complex.

The term “contacting” as used herein refers to mixing a solution comprising the test compound with a liquid medium bathing the cells of the methods. The solution comprising the compound may also comprise another component, such as dimethyl sulfoxide (DMSO), which facilitates the uptake of the test compound or compounds into the cells of the methods. The solution comprising the test compound may be added to the medium bathing the cells by utilizing a delivery apparatus, such as a pipette-based device or syringe-based device.

In another aspect, the invention features methods for identifying a substance that modulates kinase activity in a cell comprising the steps of: (a) expressing a kinase polypeptide in a cell, wherein said polypeptide is selected from the group having an amino acid sequence selected from the group consisting of those set forth in SEQ ID NO:1-87; (b) adding a test substance to said cell; and (c) monitoring a change in cell phenotype or the interaction between said polypeptide and a natural binding partner. The skilled artisan will appreciate that the kinase polypeptides of the invention, including, for example, a portion of a full-length sequence such as a catalytic domain or a portion thereof, are useful for the identification of a substance which modulates kinase activity. Those kinase polypeptides having a functional activity (e.g., catalytic activity as defined herein) are useful for identifying a substance that modulates kinase activity.

The term “expressing” as used herein refers to the production of kinases of the invention from a nucleic acid vector containing kinase genes within a cell. The nucleic acid vector is transfected into cells using well known techniques in the art as described herein.

Another aspect of the instant invention is directed to methods of identifying compounds that bind to kinase polypeptides of the present invention, comprising contacting the kinase polypeptides with a compound, and determining whether the compound binds the kinase polypeptides. Binding can be determined by binding assays which are well known to the skilled artisan, including, but not limited to, gel-shift assays, Western blots, radiolabeled competition assay, phage-based expression cloning, co-fractionation by chromatography, co-precipitation, cross linking, interaction trap/two-hybrid analysis, southwestern analysis, ELISA, and the like, which are described in, for example, Current Protocols in Molecular Biology, 1999, John Wiley & Sons, NY, which is incorporated herein by reference in its entirety. The compounds to be screened include, but are not limited to, compounds of extracellular, intracellular, biological or chemical origin.

The methods of the invention also embrace compounds that are attached to a label, such as a radiolabel (e.g., ¹²⁵I, ³⁵S, ³²P, ³³P, ³H), a fluorescence label, a chemiluminescent label, an enzymic label and an immunogenic label. The kinase polypeptides employed in such a test may either be free in solution, attached to a solid support, borne on a cell surface, located intracellularly or associated with a portion of a cell. One skilled in the art can, for example, measure the formation of complexes between a kinase polypeptide and the compound being tested. Alternatively, one skilled in the art can examine the diminution in complex formation between a kinase polypeptide and its substrate caused by the compound being tested.

Other assays can be used to examine enzymatic activity including, but not limited to, photometric, radiometric, HPLC, electrochemical, and the like, which are described in, for example, Enzyme Assays: A Practical Approach, eds. R. Eisenthal and M. J. Danson, 1992, Oxford University Press, which is incorporated herein by reference in its entirety.

Another aspect of the present invention is directed to methods of identifying compounds which modulate (i.e., increase or decrease) activity of a kinase polypeptide comprising contacting the kinase polypeptide with a compound, and determining whether the compound modifies activity of the kinase polypeptide. As described herein, the kinase polypeptides of the invention include a portion of a full-length sequence, such as a catalytic domain, as defined herein. In some instances, the kinase polypeptides of the invention comprise less than the entire catalytic domain, yet exhibit kinase or kinase-like activity. These compounds are also referred to as “modulators of protein kinases.” The activity in the presence of the test compound is measured to the activity in the absence of the test compound. Where the activity of a sample containing the test compound is higher than the activity in a sample lacking the test compound, the compound will have increased the activity. Similarly, where the activity of a sample containing the test compound is lower than the activity in the sample lacking the test compound, the compound will have inhibited the activity.

The present invention is particularly useful for screening compounds by using a kinase polypeptide in any of a variety of drug screening techniques. The compounds to be screened include, but are not limited to, extracellular, intracellular, biological or/chemical origin. The kinase polypeptide employed in such a test may be in any form, preferably, free in solution, attached to a solid support, borne on a cell surface or located intracellularly. One skilled in the art can, for example, measure the formation of complexes between a kinase polypeptide and the compound being tested. Alternatively, one skilled in the art can examine the diminution in complex formation between a kinase polypeptide and its substrate caused by the compound being tested.

The activity of kinase polypeptides of the invention can be determined by, for example, examining the ability to bind or be activated by chemically synthesised peptide ligands. Alternatively, the activity of the kinase polypeptides can be assayed by examining their ability to bind metal ions such as calcium, hormones, chemokines, neuropeptides, neurotransmitters, nucleotides, lipids, odorants, and photons. Thus, modulators of the kinase polypeptide's activity may alter a kinase function, such as a binding property of a kinase or an activity such as signal transduction or membrane localization.

In various embodiments of the method, the assay may take the form of a yeast growth assay, an Aequorin assay, a Luciferase assay, a mitogenesis assay, a MAP Kinase activity assay, as well as other binding or function-based assays of kinase activity that are generally known in the art. Biological activities of kinases according to the invention include, but are not limited to, the binding of a natural or a synthetic ligand, as well as any one of the functional activities of kinases known in the art. Non-limiting examples of kinase activities include transmembrane signaling of various forms, which may involve kinase binding interactions and/or the exertion of an influence over signal transduction.

The modulators of the invention exhibit a variety of chemical structures, which can be generally grouped into mimetics of natural kinase ligands, and peptide and non-peptide allosteric effectors of kinases. The invention does not restrict the sources for suitable modulators, which may be obtained from natural sources such as plant, animal or mineral extracts, or non-natural sources such as small molecule libraries, including the products of combinatorial chemical approaches to library construction, and peptide libraries.

The use of cDNAs encoding kinases in drug discovery programs is-well-known; assays capable of testing thousands of unknown compounds per day in high-throughput screens (HTSs) are thoroughly documented. The literature is replete with examples of the use of radiolabelled ligands in HTS binding assays for drug discovery (see Williams, Medicinal Research Reviews, 1991, 11, 147-184.; Sweetnam, et al., J. Natural Products, 1993, 56, 441-455 for review). Recombinant receptors are preferred for binding assay HTS because they allow for better specificity (higher relative purity), provide the ability to generate large amounts of receptor material, and can be used in a broad variety of formats (see Hodgson, Bio/Technology, 1992, 10, 973-980; each of which is incorporated herein by reference in its entirety).

A variety of heterologous systems is available for functional expression of recombinant receptors that are well known to those skilled in the art. Such systems include bacteria (Strosberg, et al., Trends in Pharmacological Sciences, 1992, 13, 95-98), yeast (Pausch, Trends in Biotechnology, 1997, 15, 487-494), several kinds of insect cells (Vanden Broeck, Int. Rev. Cytology, 1996, 164, 189-268), amphibian cells (Jayawickreme et al., Current Opinion in Biotechnology, 1997, 8, 629-634) and several mammalian cell lines (CHO, HEK293, COS, etc.; see Gerhardt, et al., Eur. J. Pharmacology, 1997, 334, 1-23). These examples do not preclude the use of other possible cell expression systems, including cell lines obtained from nematodes (PCT application WO 98/37177).

An expressed kinase can be used for HTS binding assays in conjunction with its defined ligand, in this case the corresponding peptide that activates it. The identified peptide is labeled with a suitable radioisotope, including, but not limited to, ¹²⁵I, ³H, ³⁵S or ³²P, by methods that are well known to those skilled in the art. Alternatively, the peptides may be labeled by well-known methods with a suitable fluorescent derivative (Baindur, et al., Drug Dev. Res., 1994, 33, 373-398; Rogers, Drug Discovery Today, 1997, 2, 156-160). Radioactive ligand specifically bound to the receptor in membrane preparations made from the cell line expressing the recombinant protein can be detected in HTS assays in one of several standard ways, including filtration of the receptor-ligand complex to separate bound ligand from unbound ligand (Williams, Med. Res. Rev., 1991, 11, 147-184.; Sweetnam, et al., J. Natural Products, 1993, 56, 441-455). Alternative methods include a scintillation proximity assay (SPA) or a FlashPlate format in which such separation is unnecessary (Nakayama, Cur. Opinion Drug Disc. Dev., 1998, 1, 85-91 Bossé, et al., J. Biomolecular Screening, 1998, 3, 285-292.). Binding of fluorescent ligands can be detected in various ways, including fluorescence energy transfer (FRET), direct spectrophotofluorometric analysis of bound ligand, or fluorescence polarization (Rogers, Drug Discovery Today, 1997, 2, 156-160; Hill, Cur. Opinion Drug Disc. Dev., 1998, 1, 92-97).

The kinases and natural binding partners required for functional expression of heterologous kinase polypeptides can be native constituents of the host cell or can be introduced through well-known recombinant technology. The kinase polypeptides can be intact or chimeric. The kinase activation results in the stimulation or inhibition of other native proteins, events that can be linked to a measurable response.

Examples of such biological responses include, but are not limited to, the following: the ability to survive in the absence of a limiting nutrient in specifically engineered yeast cells (Pausch, Trends in Biotechnology, 1997, 15, 487-494); changes in intracellular Ca²⁺ concentration as measured by fluorescent dyes (Murphy, et al., Cur. Opinion Drug Disc. Dev., 1998, 1, 192-199). Fluorescence changes can also be used to monitor ligand-induced changes in membrane potential or intracellular pH; an automated system suitable for HTS has been described for these purposes (Schroeder, et al., J. Biomolecular Screening, 1996, 1, 75-80).

The invention contemplates a multitude of assays to screen and identify inhibitors of ligand binding to kinase polypeptides. In one example, the kinase polypeptide is immobilized and interaction with a binding partner is assessed in the presence and absence of a candidate modulator such as an inhibitor compound. In another example, interaction between the kinase polypeptide and its binding partner is assessed in a solution assay, both in the presence and absence of a candidate inhibitor compound. In either assay, an inhibitor is identified as a compound that decreases binding between the kinase polypeptide and its natural binding partner. Another contemplated assay involves a variation of the di-hybrid assay wherein an inhibitor of protein/protein interactions is identified by detection of a positive signal in a transformed or transfected host cell, as described in PCT publication number WO 95/20652, published Aug. 3, 1995 and is included by reference herein including any figures, tables, or drawings.

Candidate modulators contemplated by the invention include compounds selected from libraries of either potential activators or potential inhibitors. There are a number of different libraries used for the identification of small molecule modulators, including: (1) chemical libraries, (2) natural product libraries, and (3) combinatorial libraries comprised of random peptides, oligonucleotides or organic molecules. Chemical libraries consist of random chemical structures, some of which are analogs of known compounds or analogs of compounds that have been identified as “hits” or “leads” in other drug discovery screens, while others are derived from natural products, and still others arise from non-directed synthetic organic chemistry. Natural product libraries are collections of microorganisms, animals, plants, or marine organisms which are used to create mixtures for screening by: (1) fermentation and extraction of broths from soil, plant or marine microorganisms or (2) extraction of plants or marine organisms. Natural product libraries include polyketides, non-ribosomal peptides, and variants (non-naturally occurring) thereof. For a review, see Science 282:63-68 (1998). Combinatorial libraries are composed of large numbers of peptides, oligonucleotides, or organic compounds as a mixture. These libraries are relatively easy to prepare by traditional automated synthesis methods, PCR, cloning, or proprietary synthetic methods. Of particular interest are non-peptide combinatorial libraries. Still other libraries of interest include peptide, protein, peptidomimetic, multiparallel synthetic collection, recombinatorial, and polypeptide libraries. For a review of combinatorial chemistry and libraries created therefrom, see Myers, Curr. Opin. Biotechnol. 8:701-707 (1997). Identification of modulators through use of the various libraries described herein permits modification of the candidate “hit” (or “lead”) to optimize the capacity of the “hit” to modulate activity.

Still other candidate inhibitors contemplated by the invention can be designed and include soluble forms of binding partners, as well as such binding partners as chimeric, or fusion, proteins. A “binding partner” as used herein broadly encompasses both natural binding partners as described above as well as chimeric polypeptides, peptide modulators other than natural ligands, antibodies, antibody fragments, and modified compounds comprising antibody domains that are, immunospecific for the expression product of the identified kinase gene.

Other assays may be used to identify specific peptide ligands of a kinase polypeptide, including assays that identify ligands of the target protein through measuring direct binding of test ligands to the target protein, as well as assays that identify ligands of target proteins through affinity ultrafiltration with ion spray mass spectroscopy/HPLC methods or other physical and analytical methods. Alternatively, such binding interactions are evaluated indirectly using the yeast two-hybrid system described in Fields et al., Nature, 340:245-246 (1989), and Fields et al., Trends in Genetics, 10:286-292 (1994), both of which are incorporated herein by reference. The two-hybrid system is a genetic assay for detecting interactions between two proteins or polypeptides. It can be used to identify proteins that bind to a known protein of interest, or to delineate domains or residues critical for an interaction. Variations on this methodology have been developed to clone genes that encode DNA binding proteins, to identify peptides that bind to a protein, and to screen for drugs. The two-hybrid system exploits the ability of a pair of interacting proteins to bring a transcription activation domain into close proximity with a DNA binding domain that binds to an upstream activation sequence (UAS) of a reporter gene, and is generally performed in yeast. The assay requires the construction of two hybrid genes encoding (1) a DNA-binding domain that is fused to a first protein and (2) an activation domain fused to a second protein. The DNA-binding domain targets the first hybrid protein to the UAS of the reporter gene; however, because most proteins lack an activation domain, this DNA-binding hybrid protein does not activate transcription of the reporter gene. The second hybrid protein, which contains the activation domain, cannot by itself activate expression of the reporter gene because it does not bind the UAS. However, when both hybrid proteins are present, the noncovalent interaction of the first and second proteins tethers the activation domain to the UAS, activating transcription of the reporter gene. For example, when the first protein is a kinase gene product, or fragment thereof, that is known to interact with another protein or nucleic acid, this assay can be used to detect agents that interfere with the binding interaction. Expression of the reporter gene is monitored as different test agents are added to the system. The presence of an inhibitory agent results in lack of a reporter signal.

When the function of the kinase polypeptide gene product is unknown and no ligands are known to bind the gene product, the yeast two-hybrid assay can also be used to identify proteins that bind to the gene product. In an assay to identify proteins that bind to a kinase polypeptide, or fragment thereof, a fusion polynucleotide encoding both a kinase polypeptide (or fragment) and a UAS binding domain (i.e., a first protein) may be used. In addition, a large number of hybrid genes each encoding a different second protein fused to an activation domain are produced and screened in the assay. Typically, the second protein is encoded by one or more members of a total cDNA or genomic DNA fusion library, with each second protein coding region being fused to the activation domain. This system is applicable to a wide variety of proteins, and it is not even necessary to know the identity or function of the second binding protein. The system is highly sensitive and can detect interactions not revealed by other methods; even transient interactions may trigger transcription to produce a stable mRNA that can be repeatedly translated to yield the reporter protein.

Other assays may be used to search for agents that bind to the target protein. One such screening method to identify direct binding of test ligands to a target protein is described in U.S. Pat. No. 5,585,277, incorporated herein by reference. This method relies on the principle that proteins generally exist as a mixture of folded and unfolded states, and continually alternate between the two states. When a test ligand binds to the folded form of a target protein (i.e., when the test ligand is a ligand of the target protein), the target protein molecule bound by the ligand remains in its folded state. Thus, the folded target protein is present to a greater extent in the presence of a test ligand which binds the target protein, than in the absence of a ligand. Binding of the ligand to the target protein can be determined by any method which distinguishes between the folded and unfolded states of the target protein. The function of the target protein need not be known in order for this assay to be performed. Virtually any agent can be assessed by this method as a test ligand, including, but not limited to, metals, polypeptides, proteins, lipids, polysaccharides, polynucleotides and small organic molecules.

Another method for identifying ligands of a target protein is described in Wieboldt et al., Anal. Chem., 69:1683-1691 (1997), incorporated herein by reference. This technique screens combinatorial libraries of 20-30 agents at a time in solution phase for binding to the target protein. Agents that bind to the target protein are separated from other library components by simple membrane washing. The specifically selected molecules that are retained on the filter are subsequently liberated from the target protein and analyzed by HPLC and pneumatically assisted electrospray (ion spray) ionization mass spectroscopy. This procedure selects library components with the greatest affinity for the target protein, and is particularly useful for small molecule libraries.

In preferred embodiments of the invention, methods of screening for compounds which modulate kinase activity comprise contacting test compounds with kinase polypeptides and assaying for the presence of a complex between the compound and the kinase polypeptide. In such assays, the ligand is typically labelled. After suitable incubation, free ligand is separated from that present in bound form, and the amount of free or uncomplexed label is a measure of the ability of the particular compound to bind to the kinase polypeptide.

In another embodiment of the invention, high throughput screening for compounds having suitable binding affinity to kinase polypeptides is employed. Briefly, large numbers of different small peptide test compounds are synthesised on a solid substrate. The peptide test compounds are contacted with the kinase polypeptide and washed. Bound kinase polypeptide is then detected by methods well known in the art. Purified polypeptides of the invention can also be coated directly onto plates for use in the aforementioned drug screening techniques. In addition, non-neutralizing antibodies can be used to capture the protein and immobilize it on the solid support.

Other embodiments of the invention comprise using competitive screening assays in which neutralizing antibodies capable of binding a polypeptide of the invention specifically compete with a test compound for binding to the polypeptide. In this manner, the antibodies can be used to detect the presence of any peptide that shares one or more antigenic determinants with a kinase polypeptide. Radiolabeled competitive binding studies are described in A. H. Lin et al. Antimicrobial Agents and Chemotherapy, 1997, vol. 41, no. 10. pp. 2127-2131, the disclosure of which is incorporated herein by reference in its entirety.

In another aspect, the invention provides methods for treating a disease by administering to a patient in need of such treatment a substance that modulates the activity of a kinase polypeptide selected from the group consisting of those set forth in SEQ ID NO:1-87, as well as the full-length polypeptide thereof.

In preferred embodiments, the invention provides methods for treating or preventing a disease or disorder by administering to a patient in need of such treatment a substance that modulates the activity of a kinase polypeptide having an amino acid sequence selected from the group consisting of those set forth in SEQ ID NO:1-87, as well as the full-length polypeptide thereof.

Preferably, the disease is selected from the group consisting of cancers, immune-related diseases and disorders, cardiovascular disease, brain or neuronal-associated diseases, and metabolic disorders. More specifically these diseases include cancer of tissues, blood, or hematopoietic origin, particularly those involving breast, colon, lung, prostate, cervical, brain, ovarian, bladder, or kidney; central or peripheral nervous system diseases and conditions including migraine, pain, sexual dysfunction, mood disorders, attention disorders, cognition disorders, hypotension, and hypertension; psychotic and neurological disorders, including anxiety, schizophrenia, manic depression, delirium, dementia, severe mental retardation and dyskinesias, such as Huntington's disease or Tourette's Syndrome; neurodegenerative diseases including Alzheimer's, Parkinson's, Multiple sclerosis, and Amyotrophic lateral sclerosis; viral or non-viral infections caused by HIV-1, HIV-2 or other viral- or prion-agents or fungal- or bacterial-organisms; metabolic disorders including Diabetes and obesity and their related syndromes, among others; cardiovascular disorders including reperfusion restenosis, coronary thrombosis, clotting disorders, unregulated cell growth disorders, atherosclerosis; ocular disease including glaucoma, retinopathy, and macular degeneration; inflammatory disorders including rheumatoid arthritis, chronic inflammatory bowel disease, chronic inflammatory pelvic disease, multiple sclerosis, asthma, osteoarthritis, psoriasis, atherosclerosis, rhinitis, autoimmunity, and organ transplant rejection.

Substances useful for treatment of kinase-related disorders or diseases preferably show positive results in one or more in vitro assays for an activity corresponding to treatment of the disease or disorder in question (Examples of such assays are provided throughout this application; see for example, Example 12). Examples of substances that can be screened for favorable activity are provided and referenced in section VI, below. The substances that modulate the activity of the kinases preferably include, but are not limited to, antisense oligonucleotides, ribozymes, molecules that result in RNA interference (such as double stranded RNA (dsRNA), short interfering Rna (siRNA), small temporal RNA (stRNA)) and inhibitors of protein kinases, as determined by methods and screens referenced in section VI and Example 7, below. For a discussion of RNAi, see, for example, McManus, et al. Nature Reviews Genetics 3:737 (2002).

The term “preventing” refers to decreasing the probability that an organism contracts or develops an abnormal condition.

The term “treating” refers to having a therapeutic effect and at least partially alleviating or abrogating an abnormal condition in the organism.

The term “therapeutic effect” refers to the inhibition or activation factors causing or contributing to the abnormal condition. A therapeutic effect relieves to some extent one or more of the symptoms of the abnormal condition. In reference to the treatment of abnormal conditions, a therapeutic effect can refer to one or more of the following: (a) an increase or decrease in the proliferation, growth, and/or differentiation of cells; (b) inhibition or increasing (i.e., slowing or stopping) of cell death; (c) inhibition of degeneration; (d) relieving to some extent one or more of the symptoms associated with the abnormal condition; and (e) enhancing the function of the affected population of cells. Compounds demonstrating efficacy against abnormal conditions can be identified as described herein.

The term “abnormal condition” refers to a function in the cells or tissues of an organism that deviates from their normal functions in that organism. An abnormal condition can relate to cell proliferation, cell differentiation, or cell survival. An abnormal condition may also include irregularities in cell cycle progression, i.e., irregularities in normal cell cycle progression through mitosis and meiosis.

Abnormal cell proliferative conditions include, but are not limited to, cancers such as fibrotic and mesangial disorders, abnormal angiogenesis and vasculogenesis, wound healing, psoriasis, diabetes mellitus, and inflammation.

Abnormal differentiation conditions include, but are not limited to neurodegenerative disorders, slow wound healing rates, and slow tissue grafting healing rates.

Abnormal cell survival conditions relate to conditions in which programmed cell death (apoptosis) pathways are activated or abrogated. A number of protein kinases are associated with the apoptosis pathways. Aberrations in the function of any one of the protein kinases could lead to cell immortality or premature cell death.

The term “aberration”, in conjunction with the function of a kinase in a signal transduction process, refers to a kinase that is over- or under-expressed in an organism, mutated such that its catalytic activity is lower or higher than wild-type protein kinase activity, mutated such that it can no longer interact with a natural binding partner, is no longer modified by another protein kinase or protein phosphatase, or no longer interacts with a natural binding partner.

The term “administering” relates to a method of incorporating a compound into cells or tissues of an organism. The abnormal condition can be prevented or treated when the cells or tissues of the organism exist within the organism or outside of the organism. Cells existing outside the organism can be maintained or grown in cell culture dishes. For cells harbored within the organism, many techniques exist in the art to administer compounds, including (but not limited to) oral, parenteral, dermal, injection, and aerosol applications. For cells outside of the organism, multiple techniques exist in the art to administer the compounds, including (but not limited to) cell microinjection techniques, transformation techniques, and carrier techniques.

The abnormal condition can also be prevented or treated by administering a compound to a group of cells having an aberration in a signal transduction pathway to an organism. The effect of administering a compound on organism function can then be monitored. The organism is preferably a mouse, rat, rabbit, guinea pig, or goat, more preferably a monkey or ape, and most preferably a human.

In another aspect, the invention features methods for detection of a kinase polypeptide in a sample as a diagnostic tool for diseases or disorders, wherein the method comprises the steps of: (a) contacting the sample with a nucleic acid probe which hybridizes under hybridization assay conditions to a nucleic acid target region of a kinase polypeptide, wherein the kinase polypeptide has an amino acid sequence selected from the group consisting of those set forth in SEQ ID NO:1-87, said probe comprising the nucleic acid sequence encoding the polypeptide, fragments thereof, and the complements of the sequences and fragments; and (b) detecting the presence or amount of the probe:target region hybrid as an indication of the disease. The nucleic acid target region can also be selected from the nucleic acids of SEQ ID NO:88-174.

In preferred embodiments of the invention, the disease or disorder is selected from the group consisting of rheumatoid arthritis, arteriosclerosis, autoimmune disorders, organ transplantation, myocardial infarction, cardiomyopathies, stroke, renal failure, oxidative stress-related neurodegenerative disorders, and cancer.

The kinase “target region” is the nucleotide base sequence selected from the group consisting of those set forth in SEQ ID NO:88-175, or the corresponding full-length sequences, a functional derivative thereof, or a fragment thereof, to which the nucleic acid probe will specifically hybridize. Specific hybridization indicates that in the presence of other nucleic acids the probe only hybridizes detectably with the kinase of the invention's target region. Putative target regions can be identified by methods well known in the art consisting of alignment and comparison of the most closely related sequences in the database.

In preferred embodiments the nucleic acid probe hybridizes to a kinase target region encoding at least 6, 12, 75, 90, 105, 120, 150, 200, 250, 300 or 350 contiguous amino acids of a sequence selected from the group consisting of those set forth in SEQ ID NO:1-87, or the corresponding full-length amino acid sequence, a portion of any of these sequences that retains functional activity, as described herein, or a functional derivative thereof. Hybridization conditions should be such that hybridization occurs only with the kinase genes in the presence of other nucleic acid molecules. Under stringent hybridization conditions only highly complementary nucleic acid sequences hybridize. Preferably, such conditions prevent hybridization of nucleic acids having more than 1 or 2 mismatches out of 20 contiguous nucleotides. Such conditions are defined supra.

The diseases for which detection of kinase genes in a sample could be diagnostic include diseases in which kinase nucleic acid (DNA and/or RNA) is amplified in comparison to normal cells. By “amplification” is meant increased numbers of kinase DNA or RNA in a cell compared with normal cells. In normal cells, kinases are typically found as single copy genes. In selected diseases, the chromosomal location of the kinase genes may be amplified, resulting in multiple copies of the gene, or amplification. Gene amplification can lead to amplification of kinase RNA, or kinase RNA can be amplified in the absence of kinase DNA amplification.

“Amplification” as it refers to RNA can be the detectable presence of kinase RNA in cells, since in some normal cells there is no basal expression of kinase RNA. In other normal cells, a basal level of expression of kinase exists, therefore in these cases amplification is the detection of at least 1-2-fold, and preferably more, kinase RNA, compared to the basal level.

The test samples suitable for nucleic acid probing methods of the present invention include, for example, cells or nucleic acid extracts of cells, or biological fluids. The samples used in the above-described methods will vary based on the assay format, the detection method and the nature of the tissues, cells or extracts to be assayed. Methods for preparing nucleic acid extracts of cells are well known in the art and can be readily adapted in order to obtain a sample that is compatible with the method utilized.

The invention also features a method for detection of a kinase polypeptide in a sample as a diagnostic tool for a disease or disorder, wherein the method comprises: (a) comparing a nucleic acid target region encoding the kinase polypeptide in a sample, where the kinase polypeptide has an amino acid sequence selected from the group consisting those set forth in SEQ ID NO:1-87, or one or more fragments thereof, with a control nucleic acid target region encoding the kinase polypeptide, or one or more fragments thereof; and (b) detecting differences in sequence or amount between the target region and the control target region, as an indication of the disease or disorder. Preferably the disease is selected from the group consisting of cancers, immune-related diseases and disorders, cardiovascular disease, brain or neuronal-associated diseases, and metabolic disorders. More specifically these diseases include cancer of tissues, blood, or hematopoietic origin, particularly those involving breast, colon, lung, prostate, cervical, brain, ovarian, bladder, or kidney; central or peripheral nervous system diseases and conditions including migraine, pain, sexual dysfunction, mood disorders, attention disorders, cognition disorders, hypotension, and hypertension; psychotic and neurological disorders, including anxiety, schizophrenia, manic depression, delirium, dementia, severe mental retardation and dyskinesias, such as Huntington's disease or Tourette's Syndrome; neurodegenerative diseases including Alzheimer's, Parkinson's, Multiple sclerosis, and Amyotrophic lateral sclerosis; viral or non-viral infections caused by HIV-1, HIV-2 or other viral- or prion-agents or fungal- or bacterial-organisms; metabolic disorders including Diabetes and obesity and their related syndromes, among others; cardiovascular disorders including reperfusion restenosis, coronary thrombosis, clotting disorders, unregulated cell growth disorders, atherosclerosis; ocular disease including glaucoma, retinopathy, and macular degeneration; inflammatory disorders including rheumatoid arthritis, chronic inflammatory bowel disease, chronic inflammatory pelvic disease, multiple sclerosis, asthma, osteoarthritis, psoriasis, atherosclerosis, rhinitis, autoimmunity, and organ transplant rejection.

The term “comparing” as used herein refers to identifying discrepancies between the nucleic acid target region isolated from a sample, and the control nucleic acid target region. The discrepancies can be in the nucleotide sequences, e.g. insertions, deletions, or point mutations, or in the amount of a given nucleotide sequence. Methods to determine these discrepancies in sequences are well-known to one of ordinary skill in the art. The “control” nucleic acid target region refers to the sequence or amount of the sequence found in normal cells, e.g. cells that are not diseased as discussed previously.

The summary of the invention described above is not limiting and other features and advantages of the invention will be apparent from the following detailed description of the invention, and from the claims.

### BRIEF DESCRIPTION OF THE FIGURES

FIGS. 1A and 1B show the structural neighbors of 1ia9A.

FIG. 1C shows a CRISSP prototype for protein kinases.

FIG. 1D shows a CRISSP prototype for protein phosphatases.

FIG. 1E shows a CRISSP prototype for nuclear hormone receptors.

FIG. 2 shows the amino acid sequences of 87 kinases of the invention, along with the predicted secondary structure. CRISSPs are also shown, (corresponding to SEQ ID NO:1-87).

FIG. 3 shows the amino acid sequences of 87 kinases of the invention (corresponding to SEQ ID NO:1-87).

FIG. 4 shows the nucleic acid sequences of 87 kinases of the invention (coresponding to SEQ ID NO:88-174).

### DETAILED DESCRIPTION OF THE INVENTION

The present invention relates to a method for detecting remote polypeptide homologues, comprising analysis of conserved secondary structure pattern in a protein family, and conserved active site amino acid residues. The analyses are used to identify conserved residues embedded into the secondary structure pattern (CRISSP), which are used to detect remote homologues of the referent protein family. The inventive method has a low false positive rate, and can detect remote homologues that cannot be detected using sequence or secondary structure-based methods.

The invention also provides, inter alia, protein kinase and kinase-like genes, as well as fragments thereof, which have been identified in genomic databases using the CRISSP method of the invention. In part, the invention provides nucleic acid molecules that are capable of encoding polypeptides having a kinase or kinase-like activity. The invention additionally provides a number of different embodiments, such as those described below.

The method includes a method for identifying a remote polypeptide homologue to a referent protein family, comprising:

(a) identifying the conserved secondary structure pattern (CSSP) of said protein family;

(b) identifying the conserved amino acid residues (CAAR) or conserved active site amino acid residues (CASAAR) of the referent protein family; and

(c) identifying the conserved residues embedded into the secondary structure pattern (CRISSP); and

(d) identifying the candidate as a remote homologue if the candidate polypeptide contains the CRISSP of (c).

In other embodiments, the invention includes the remote polypeptide homologue detection method, wherein said referent protein family is the protein kinase family. In other embodiments, the referent protein family is the phosphatase family or the protease family. As described herein, the secondary structure pattern can be identified using DSSP and the CASAAR can be are identified using FSSP database.

The invention also includes a computer readable medium having program code stored thereon for identifying a remote polypeptide homologue to a referent protein family, the program code configured to cause a computer to perform the following steps:

(a) identifying the conserved secondary structure pattern of said protein family;

(b) identifying the conserved amino acid residues or conserved active site amino acid residues of the referent protein family; and

(c) identifying the conserved residues embedded into the secondary structure pattern (CRISSP); and

(d) identifying the candidate as a remote homologue if the candidate polypeptide contains the CRISSP of (c).

The invention also includes a programmed storage device comprising instructions that when executed perform the steps of:

(a) identifying the conserved secondary structure pattern of said protein family;

(b) identifying the conserved amino acid residues or conserved active site amino acid residues of the referent protein family; and

(c) identifying the conserved residues embedded into the secondary structure pattern (CRISSP); and (d) identifying the candidate as a remote homologue if the candidate polypeptide contains the CRISSP of (c).

The invention further includes a process for effecting analysis of a polypeptide sequence through use of a computer having a memory, said process comprising:

(a) placing into said memory data representing a polypeptide,

(b) developing within said memory a data structure associated with said data and reflecting the underlying organization and structure of the data to facilitate program access to data elements corresponding to logical sub-components of the sequence,

(c) programming said computer with a program containing instructions sufficient to implement the method of claim 1, and

(d) executing said program on said computer while granting said program access to said data and to said data structure within said memory.

Machine Applications

The present invention provides machines, data structures, and processes for analyzing the polypeptide according to the present invention.

A. Machines: Data, Data Structures, Processes, and Functions

The present invention provides a machine having a memory comprising: 1) data representing a CRISSP of the present invention, 2) a data structure which reflects the underlying organization and structure of the data and facilitates program access to data elements corresponding to logical sub-components of CRISSP and polypeptide sequences, 3) processes for effecting the use, analysis, or modeling of the CRISSP, and 4) optionally, a function or utility for CRISSP and polypeptides having the CRISSP.

The machine of the present invention is typically a digital computer. The term “computer” includes one or several desktop or portable computers, computer workstations, servers (including intranet or internet servers), mainframes, and any integrated system comprising any of the above irrespective of whether the processing, memory, input, or output of the computer is remote or local, as well as any networking interconnecting the modules of the computer. The term “computer” is exclusive of computers of the United States patent and Trademark Office or the European Patent Office when data representing the CRISSP of the present invention is used for patentability searches.

The present invention contemplates providing as data a CRISSP of the present invention embodied in a computer readable medium. As those of skill in the art will be aware, the form of memory of a machine of the present invention, or the particular embodiment of the computer readable medium, are not critical elements of the invention and can take a variety of forms. The memory of such a machine includes, but is not limited to, ROM, or RAM, or computer readable media such as, but not limited to, magnetic media such as computer disks or hard drives, or media such as CD-ROMs, DVDs, and the like.

The present invention further contemplates providing a data structure that is also contained in memory. The data structure may be defined by the computer programs that define the processes (see below) or it may be defined by the programming of separate data storage and retrieval programs subroutines, or systems.

In one embodiment, the present invention provides a data structure that contains data representing a CRISSP of the present invention stored within a computer readable medium. The data structure is organized to reflect the logical structuring of the sequence, so that the sequence is easily analyzed by software programs capable of accessing the data structure. In particular, the data structures of the present invention organize the reference sequences of the present invention in a manner which allows software tools to perform a wide variety of analyses using logical elements and sub-elements of each sequence.

This data structure is an open structure and is robust enough to accommodate newly generated data and acquired knowledge. Such a structure is also a flexible structure. It can be trimmed down to a l-D string to facilitate data mining and analysis steps, such as clustering, repeat-masking, and HMM (Hidden Markov Model) analysis. Meanwhile, such a data structure also can extend the associated attributes into multiple dimensions. Pointers can be established among the dimensioned attributes when needed to facilitate data management and processing in a comprehensive genomics knowledgebase. Furthermore, such a data structure is object-oriented. Polymorphism can be represented by a family or class of sequence objects, each of which has an internal structure as discussed above. The common traits are abstracted and assigned to the parent object, whereas each child object represents a specific variant of the family or class. Such a data structure allows data to be efficiently retrieved, updated and integrated by the software applications associated with the sequence database and/or knowledgebase.

Optionally, the present invention further contemplates that the machine of the present invention will embody in some manner a utility or function for the CRISSP of the present invention. The function or utility of the CRISSP can be a function or utility for a polypeptide sequence having the CRISSP, per se, or of the tangible material. Exemplary function or utilities include the name (per International Union of Biochemistry and Molecular Biology rules of nomenclature) or function of the enzyme or protein represented by a polypeptide having the CRISSP of the present invention; the metabolic pathway of the protein represented by a polypeptide having the CRISSP of the present invention; the substrate or product or structural role of the protein represented by a polypeptide polypeptide having the CRISSP of the present invention; or, the phenotype (e.g., an agronomic or pharmacological trait) affected by modulating expression or activity of the protein represented by a polypeptide having the CRISSP of the present invention.

B. Computer Analysis and Modeling

The present invention provides a process of modeling and analyzing data using the novel method of the invention. The process comprises entering a CRISSP and sequence data of polypeptides into a machine having a hardware or software sequence modeling and analysis system, developing data structures to facilitate access to the sequence data, manipulating the data to model or analyze the activity of the polypeptide based on one or more CRISSPs of the invention, and displaying the results of the modeling or analysis.

In a further embodiment, additional modeling an analytical tools can be used in conjunction with the novel methods of the invention. A variety of modeling and analytic tools are well known in the art and available commercially. Included amongst the modeling/analysis tools are methods to: 1) backtranslate polypeptides having a CRISSP of the present invention into polynucleotides and perform analysis of the polynucleotides including but not limited to recognizing overlapping sequences (e.g., from a sequencing project) with the polynucleotide to create an alignment called a “contig”, identifying restriction enzyme sites, identifying PCR primers with minimal self-complementarity; 2) compute pairwise distances between polypeptides having a CRISSP and the corresponding polynucleotide sequences in an alignment, reconstruct phylogentic trees using distance methods, and calculate the degree of divergence of two protein coding regions; 3) identify sequence patterns, functional motifs and signatures, known functional domains in polypeptides having a CRISSP of the present invention; 4) identify structural patterns, structure-based functional motifs and signatures, functional domains of known structure in protein structures representing a referent protein family of the present invention; 5) identify or predict various biochemical and structural properties of the polypeptides having CRISSP of the present invention such as isoelectric point, secondary structure, hydrophobicity, and antigenicity 6) analyze physical properties such as hydrophobicity, electrical charge distribution and curvature of the surfaces of protein structures representing a referent protein family of the present invention 7) compare two or more protein or nucleic acid sequences and identifying points of similarity or dissimilarity between them in one-dimensional space; 8) compare two or more protein structures and identifying points of similarity or dissimilarity between them in three-dimensional space; 9) recognize protein fold using various techniques including but not limited to lD- and 3D-threading (as discussed in introductions), aligning of sequences and 3D structures, predicted secondary structures, and combination of sequence and secondary structure information, Hidden Markov Models, Neural Networks, Support Vector Machines; 10) build 3D structural models of a polypeptide having CRISSP of the present invention; 11) apply protein docking techniques to model interactions between proteins or small proteins and polypeptides having CRISSP of the present invention.

The processes for effecting analysis and modeling can be produced independently or obtained from commercial suppliers. Exemplary analysis and modeling tools are provided in products such as InforMax's (Bethesda, Md.) Vector NTI Suite (Version 5.5), Intelligenetics' (Mountain View, Calif.) PC/Gene program, Genetics Computer Group's (Madison, Wis.) Wisconsin Package (Version 10.0), and Accelrys's (San Diego, Calif.) Insight II package.

The invention includes a process for effecting the use, analysis, or modeling of a polynucleotide sequence or its derived peptide sequence through use of a computer having a memory, said process comprising:

placing into said memory data representing a CRISSP,

developing within said memory a data structure associated with said data and reflecting the underlying organization and structure of the data to facilitate program access to data elements corresponding to logical sub-components of the sequence,

programming said computer with a program containing instructions sufficient to implement the process for effecting the use, analysis, or modeling of said polynucleotide sequence or said peptide sequence, and

executing said program on said computer while granting said program access to said data and to said data structure within said memory.

Nucleic Acids

Associations of chromosomal localizations for mapped genes with amplicons implicated in cancer are based on literature searches (PubMed http://www.ncbi.nlm.nih.gov/entrez/query.fcgi), OMIM searches (Online Mendelian Inheritance in Man, http://www.ncbi.nlm.nih.gov/Omim/searchomim.html) and the comprehensive database of cancer amplicons maintained by Knuutila, et al. (Knuutila, et al., DNA copy number amplifications in human neoplasms. Review of comparative genomic hybridization studies. Am J Pathol 152:1107-1123, 1998. http://www.helsinki.fi/˜lgl www/CMG.html). For many of the mapped genes, the cytogenetic region from Knuutila is listed followed by the number of cases with documented amplification and the total number of cases studied.

For single nucleotide polymorphisms, an accession number is given if the SNP is documented in dbSNP (the database of single nucleotide polymorphisms) maintained at NCBI (http://www.ncbi.nlm.nih.gov/SNP/index.html). The accession number for SNP can be used to retrieve the full SNP-containing sequence from this site.

Nucleic Acid Probes, Methods, and Kits for Detection of Kinases

The invention additionally provides nucleic acid probes and uses therefor. A nucleic acid probe of the present invention may be used to probe an appropriate chromosomal or cDNA library by usual hybridization methods to obtain other nucleic acid molecules of the present invention. A chromosomal DNA or cDNA library may be prepared from appropriate cells according to recognized methods in the art (cf. “Molecular Cloning: A Laboratory Manual”, second edition, Cold Spring Harbor Laboratory, Sambrook, Fritsch, & Maniatis, eds., 1989).

In the alternative, chemical synthesis can be carried out in order to obtain nucleic acid probes having nucleotide sequences which correspond to N-terminal and C-terminal portions of the amino acid sequence of the polypeptide of interest. The synthesized nucleic acid probes may be used as primers in a polymerase chain reaction (PCR) carried out in accordance with recognized PCR techniques, essentially according to PCR Protocols, “A Guide to Methods and Applications”, Academic Press, Michael, et al., eds., 1990, utilizing the appropriate chromosomal or cDNA library to obtain the fragment of the present invention.

One skilled in the art can readily design such probes based on the sequence disclosed herein using methods of computer alignment and sequence analysis known in the art (“Molecular Cloning: A Laboratory Manual”, 1989, supra). The hybridization probes of the present invention can be labeled by standard labeling techniques such as with a radiolabel, enzyme label, fluorescent label, biotin-avidin label, chemiluminescence, and the like. After hybridization, the probes may be visualized using known methods.

The nucleic acid probes of the present invention include RNA, as well as DNA probes, such probes being generated using techniques known in the art. The nucleic acid probe may be immobilized on a solid support. Examples of such solid supports include, but are not limited to, plastics such as polycarbonate, complex carbohydrates such as agarose and sepharose, and acrylic resins, such as polyacrylamide and latex beads. Techniques for coupling nucleic acid probes to such solid supports are well known in the art.

The test samples suitable for nucleic acid probing methods of the present invention include, for example, cells or nucleic acid extracts of cells, or biological fluids. The samples used in the above-described methods will vary based on the assay format, the detection method and the nature of the tissues, cells or extracts to be assayed. Methods for preparing nucleic acid extracts of cells are well known in the art and can be readily adapted in order to obtain a sample which is compatible with the method utilized.

One method of detecting the presence of nucleic acids of the invention in a sample comprises (a) contacting said sample with the above-described nucleic acid probe under conditions such that hybridization occurs, and (b) detecting the presence of said probe bound to said nucleic acid molecule. One skilled in the art would select the nucleic acid probe according to techniques known in the art as described above. Samples to be tested include but should not be limited to RNA samples of human tissue.

A kit for detecting the presence of nucleic acids of the invention in a sample comprises at least one container means having disposed therein the above-described nucleic acid probe. The kit may further comprise other containers comprising one or more of the following: wash reagents and reagents capable of detecting the presence of bound nucleic acid probe. Examples of detection reagents include, but are not limited to radiolabelled probes, enzymatic labeled probes (horseradish peroxidase, alkaline phosphatase), and affinity labeled probes (biotin, avidin, or steptavidin). Preferably, the kit further comprises instructions for use.

In detail, a compartmentalized kit includes any kit in which reagents are contained in separate containers. Such containers include small glass containers, plastic containers or strips of plastic or paper. Such containers allow the efficient transfer of reagents from one compartment to another compartment such that the samples and reagents are not cross-contaminated and the agents or solutions of each container can be added in a quantitative fashion from one compartment to another. Such containers will include a container which will accept the test sample, a container which contains the probe or primers used in the assay, containers which contain wash reagents (such as phosphate buffered saline, Tris-buffers, and the like), and containers which contain the reagents used to detect the hybridized probe, bound antibody, amplified product, or the like. One skilled in the art will readily recognize that the nucleic acid probes described in the present invention can readily be incorporated into one of the established kit formats which are well known in the art.

### Categorization of the Polypeptides According to the Invention

To characterize kinases, there may be provided a classification of the protein class and family to which it belongs, a summary of non-catalytic protein motifs, as well as a chromosomal location. This information is useful in determing function, regulation and/or therapeutic utility for each of the proteins. Amplification of chromosomal region can be associated with various cancers. For amplicons discussed in this application, the source of information was Knuutila, et al (Knuutila S, Björkqvist A-M, Autio K, Tarkkanen M, Wolf M, Monni O, Szymanska J, Larramendy ML, Tapper J, Pere H, El-Rifai W, Hemmer S, Wasenius V-M, Vidgren V & Zhu Y: DNA copy number amplifications in human neoplasms. Review of comparative genomic hybridization studies. Am J Pathol 152:1107-1123, 1998. http://www.helsinki.fi/˜lgl_www/CMG.html).

The kinase classification and protein domains often reflect pathways, cellular roles, or mechanisms of up- or down-stream regulation. Also disease-relevant genes often occur in families of related genes. For example, if one member of a kinase family functions as an oncogene, a tumor suppressor, or has been found to be disrupted in an immune, neurologic, cardiovascular, or metabolic disorder, frequently other family members may play a related role.

The expression analysis organizes kinases into groups that are transcriptionally upregulated in tumors and those that are more restricted to specific tumor types such as melanoma or prostate. This analysis also identifies genes that are regulated in a cell cycle dependent manner, and are therefore likely to be involved in maintaining cell cycle checkpoints, entry, progression, or exit from mitosis, oversee DNA repair, or are involved in cell proliferation and genome stability. Expression data also can identify genes expressed in endothelial sources or other tissues that suggest a role in angiogenesis, thereby implicating them as targets for control of diseases that have an angiogenic component, such as cancer, endometriosis, retinopathy and macular degeneration, and various ischemic or vascular pathologies. A proteins' role in cell survival can also be suggested based on restricted expression in cells subjected to external stress such as oxidative damage, hypoxia, drugs such as cisplatinum, or irradiation. Metastases-associated genes can be implicated when expression is restricted to invading regions of a tumor, or is only seen in local or distant metastases compared to the primary tumor, or when a gene is upregulated during cell culture models of invasion, migration, or motility.

Chromosomal location can identify candidate targets for a tumor amplicon or a tumor-suppressor locus. Summaries of prevalent tumor amplicons are available in the literature, and can identify tumor types to experimentally be confirmed to contain amplified copies of a kinase gene which localizes to an adjacent region.

A more specific characterization of the polypeptides of the invention, including potential biological and clinical implications, is provided.

### Functional Derivatives

Also provided herein are functional derivatives of a polypeptide or nucleic acid of the invention. By “functional derivative” is meant a “chemical derivative,” “fragment,” or “variant,” of the polypeptide or nucleic acid of the invention, which terms are defined below. A functional derivative retains at least a portion of the function of the protein, for example reactivity with an antibody specific for the protein, enzymatic activity or binding activity mediated through noncatalytic domains, which permits its utility in accordance with the present invention. It is well known in the art that due to the degeneracy of the genetic code numerous different nucleic acid sequences can code for the same amino acid sequence. Equally, it is also well known in the art that conservative changes in amino acid can be made to arrive at a protein or polypeptide that retains the functionality of the original. In both cases, all permutations are intended to be covered by this disclosure.

Included within the scope of this invention are the functional equivalents of the herein-described isolated nucleic acid molecules. The degeneracy of the genetic code permits substitution of certain codons by other codons that specify the same amino acid and hence would give rise to the same protein. The nucleic acid sequence can vary substantially since, with the exception of methionine and tryptophan, the known amino acids can be coded for by more than one codon. Thus, portions or all of the genes of the invention could be synthesized to give a nucleic acid sequence significantly different from one selected from the group consisting of those set forth in SEQ ID NO:88-175. The encoded amino acid sequence thereof would, however, be preserved.

In addition, the nucleic acid sequence may comprise a nucleotide sequence which results from the addition, deletion or substitution of at least one nucleotide to the 5′-end and/or the 3′-end of the nucleic acid formula selected from the group consisting of those set forth in SEQ ID NO:88-175, or a derivative thereof. Any nucleotide or polynucleotide may be used in this regard, provided that its addition, deletion or substitution does not alter the amino acid sequence of selected from the group consisting of those set forth in SEQ ID NO:88-175 which is encoded by the nucleotide sequence. For example, the present invention is intended to include any nucleic acid sequence resulting from the addition of ATG as an initiation codon at the 5′-end of the inventive nucleic acid sequence or its derivative, or from the addition of TTA, TAG or TGA as a termination codon at the 3′-end of the inventive nucleotide sequence or its derivative. Moreover, the nucleic acid molecule of the present invention may, as necessary, have restriction endonuclease recognition sites added to its 5′-end and/or 3′-end.

Such functional alterations of a given nucleic acid sequence afford an opportunity to promote secretion and/or processing of heterologous proteins encoded by foreign nucleic acid sequences fused thereto. All variations of the nucleotide sequence of the kinase genes of the invention and fragments thereof permitted by the genetic code are, therefore, included in this invention.

Further, it is possible to delete codons or to substitute one or more codons with codons other than degenerate codons to produce a structurally modified polypeptide, but one which has substantially the same utility or activity as the polypeptide produced by the unmodified nucleic acid molecule. As recognized in the art, the two polypeptides are functionally equivalent, as are the two nucleic acid molecules that give rise to their production, even though the differences between the nucleic acid molecules are not related to the degeneracy of the genetic code.

A “chemical derivative” of the complex contains additional chemical moieties not normally a part of the protein. Covalent modifications of the protein or peptides are included within the scope of this invention. Such modifications may be introduced into the molecule by reacting targeted amino acid residues of the peptide with an organic derivatizing agent that is capable of reacting with selected side chains or terminal residues, as described below.

Cysteinyl residues most commonly are reacted with alpha-haloacetates (and corresponding amines), such as chloroacetic acid or chloroacetamide, to give carboxymethyl or carboxyamidomethyl derivatives. Cysteinyl residues also are derivatized by reaction with bromotrifluoroacetone, chloroacetyl phosphate, N-alkylmaleimides, 3-nitro-2-pyridyl disulfide, methyl 2-pyridyl disulfide, p-chloromercuribenzoate, 2-chloromercuri-4-nitrophenol, or chloro-7-nitrobenzo-2-oxa-1,3-diazole.

Histidyl residues are derivatized by reaction with diethylprocarbonate at pH 5.5-7.0 because this agent is relatively specific for the histidyl side chain. Para-bromophenacyl bromide also is useful; the reaction is preferably performed in 0.1 M sodium cacodylate at pH 6.0.

Lysinyl and amino terminal residues are reacted with succinic or other carboxylic acid anhydrides. Derivatization with these agents has the effect or reversing the charge of the lysinyl residues. Other suitable reagents for derivatizing primary amine containing residues include imidoesters such as methyl picolinimidate; pyridoxal phosphate; pyridoxal; chloroborohydride; trinitrobenzenesulfonic acid; O-methylisourea; 2,4 pentanedione; and transaminase-catalyzed reaction with glyoxylate.

Arginyl residues are modified by reaction with one or several conventional reagents, among them phenylglyoxal, 2,3-butanedione, 1,2-cyclohexanedione, and ninhydrin. Derivatization of arginine residues requires that the reaction be performed in alkaline conditions because of the high pKₐ of the guanidine functional group. Furthermore, these reagents may react with the groups of lysine as well as the arginine alpha-amino group.

Tyrosyl residues are well-known targets of modification for introduction of spectral labels by reaction with aromatic diazonium compounds or tetranitromethane. Most commonly, N-acetylimidizoi and tetranitromethane are used to form O-acetyl tyrosyl species and 3-nitro derivatives, respectively.

Carboxyl side groups (aspartyl or glutamyl) are selectively modified by reaction with carbodiimide (R′—N—C—N—R′) such as 1-cyclohexyl-3-(2-morpholinyl(4-ethyl) carbodiimide or 1-ethyl-3-(4-azonia-4,4-dimethylpentyl) carbodiimide. Furthermore, aspartyl and glutamyl residues are converted to asparaginyl and glutaminyl residues by reaction with ammonium ions.

Glutaminyl and asparaginyl residues are frequently deamidated to the corresponding glutamyl and aspartyl residues. Alternatively, these residues are deamidated under mildly acidic conditions. Either form of these residues falls within the scope of this invention.

Derivatization with bifunctional agents is useful, for example, for cross-linking the component peptides of the protein to each other or to other proteins in a complex to a water-insoluble support matrix or to other macromolecular carriers. Commonly used cross-linking agents include, for example, 1,1-bis(diazoacetyl)-2-phenylethane, glutaraldehyde, N-hydroxysuccinimide esters, for example, esters with 4-azidosalicylic acid, homobifunctional imidoesters, including disuccinimidyl esters such as 3,3′-dithiobis(succinimidylpropionate), and bifunctional maleimides such as bis-N-maleimido-1,8-octane. Derivatizing agents such as methyl-3-[p-azidophenyl) dithiolpropioimidate yield photoactivatable intermediates that are capable of forming crosslinks in the presence of light. Alternatively, reactive water-insoluble matrices such as cyanogen bromide-activated carbohydrates and the reactive substrates described in U.S. Pat. Nos. 3,969,287; 3,691,016; 4,195,128; 4,247,642; 4,229,537; and 4,330,440 are employed for protein immobilization.

Other modifications include hydroxylation of proline and lysine, phosphorylation of hydroxyl groups of seryl or threonyl residues, methylation of the alpha-amino groups of lysine, arginine, and histidine side chains (Creighton, T. E., Proteins: Structure and Molecular Properties, W. H. Freeman & Co., San Francisco, pp. 79-86 (1983)), acetylation of the N-terminal amine, and, in some instances, amidation of the C-terminal carboxyl groups.

Such derivatized moieties may improve the stability, solubility, absorption, biological half life, and the like. The moieties may alternatively eliminate or attenuate any undesirable side effect of the protein complex and the like. Moieties capable of mediating such effects are disclosed, for example, in Remington's Pharmaceutical Sciences, 18th ed., Mack Publishing Co., Easton, Pa. (1990).

The term “fragment” is used to indicate a polypeptide derived from the amino acid sequence of the proteins, of the complexes having a length less than the full-length polypeptide from which it has been derived. Such a fragment may, for example, be produced by proteolytic cleavage of the full-length protein. Preferably, the fragment is obtained recombinantly by appropriately modifying the DNA sequence encoding the proteins to delete one or more amino acids at one or more sites of the C-terminus, N-terminus, and/or within the native sequence. Fragments of a protein are useful for screening for substances that act to modulate signal transduction, as described herein. It is understood that such fragments may retain one or more characterizing portions of the native complex. Examples of such retained characteristics include: catalytic activity; substrate specificity; interaction with other molecules in the intact cell; regulatory functions; or binding with an antibody specific for the native complex, or an epitope thereof.

Another functional derivative intended to be within the scope of the present invention is a “variant” polypeptide which either lacks one or more amino acids or contains additional or substituted amino acids relative to the native polypeptide. The variant may be derived from a naturally occurring complex component by appropriately modifying the protein DNA coding sequence to add, remove, and/or to modify codons for one or more amino acids at one or more sites of the C-terminus, N-terminus, and/or within the native sequence. It is understood that such variants having added, substituted and/or additional amino acids retain one or more characterizing portions of the native protein, as described above.

A functional derivative of a protein with deleted, inserted and/or substituted amino acid residues may be prepared using standard techniques well-known to those of ordinary skill in the art. For example, the modified components of the functional derivatives may be produced using site-directed mutagenesis techniques (as exemplified by Adelman et al., 1983, DNA 2:183) wherein nucleotides in the DNA coding the sequence are modified such that a modified coding sequence is modified, and thereafter expressing this recombinant DNA in a prokaryotic or eukaryotic host cell, using techniques such as those described above. Alternatively, proteins with amino acid deletions, insertions and/or substitutions may be conveniently prepared by direct chemical synthesis, using methods well-known in the art. The functional derivatives of the proteins typically exhibit the same qualitative biological activity as the native proteins.

### Therapeutic Methods According to the Invention

Diagnostics:

The invention provides methods for detecting a polypeptide in a sample as a diagnostic tool for diseases or disorders, wherein the method comprises the steps of: (a) contacting the sample with a nucleic acid probe which hybridizes under hybridization assay conditions to a nucleic acid target region of a polypeptide selected from the group consisting of SEQ ID NO:1-87, said probe comprising the nucleic acid sequence encoding the polypeptide, fragments thereof, and the complements of the sequences and fragments; and (b) detecting the presence or amount of the probe:target region hybrid as an indication of the disease.

In preferred embodiments of the invention, the disease or disorder is selected from the group consisting of rheumatoid arthritis, atherosclerosis, autoimmune disorders, organ transplantation, myocardial infarction, cardiomyopathies, stroke, renal failure, oxidative stress-related neurodegenerative disorders, metabolic disorder including diabetes, reproductive disorders including infertility, and cancer.

Hybridization conditions should be such that hybridization occurs only with the genes in the presence of other nucleic acid molecules. Under stringent hybridization conditions only highly complementary nucleic acid sequences hybridize. Preferably, such conditions prevent hybridization of nucleic acids having 1 or 2 mismatches out of 20 contiguous nucleotides. Such conditions are defined supra.

The diseases for which detection of genes in a sample could be diagnostic include diseases in which nucleic acid (DNA and/or RNA) is amplified in comparison to normal cells. By “amplification” is meant increased numbers of DNA or RNA in a cell compared with normal cells.

“Amplification” as it refers to RNA can be the detectable presence of RNA in cells, since in some normal cells there is no basal expression of RNA. In other normal cells, a basal level of expression exists, therefore in these cases amplification is the detection of at least 1-2-fold, and preferably more, compared to the basal level.

The diseases that could be diagnosed by detection of nucleic acid in a sample preferably include cancers. The test samples suitable for nucleic acid probing methods of the present invention include, for example, cells or nucleic acid extracts of cells, or biological fluids. The samples used in the above-described methods will vary based on the assay format, the detection method and the nature of the tissues, cells or extracts to be assayed. Methods for preparing nucleic acid extracts of cells are well known in the art and can be readily adapted in order to obtain a sample that is compatible with the method utilized.

Antibodies, Hybridomas, Methods of Use and Kits for Detection of Kinases

The present invention relates to an antibody having binding affinity to a kinase of the invention. The polypeptide may have the amino acid sequence selected from the group consisting of those set forth in SEQ ID NO:3 or 4, or a functional derivative thereof, or at least 9 contiguous amino acids thereof (preferably, at least 20, 30, 35, or 40 contiguous amino acids thereof).

The present invention also relates to an antibody having specific binding affinity to a kinase of the invention. Such an antibody may be isolated by comparing its binding affinity to a kinase of the invention with its binding affinity to other polypeptides. Those which bind selectively to a kinase of the invention would be chosen for use in methods requiring a distinction between a kinase of the invention and other polypeptides. Such methods could include, but should not be limited to, the analysis of altered kinase expression in tissue containing other polypeptides.

The kinases of the present invention can be used in a variety of procedures and methods, such as for the generation of antibodies, for use in identifying pharmaceutical compositions, and for studying DNA/protein interaction.

The kinases of the present invention can be used to produce antibodies or hybridomas. One skilled in the art will recognize that if an antibody is desired, such a peptide could be generated as described herein and used as an immunogen. The antibodies of the present invention include monoclonal and polyclonal antibodies, as well fragments of these antibodies, and humanized forms. Humanized forms of the antibodies of the present invention may be generated using one of the procedures known in the art such as chimerization or CDR grafting.

The present invention also relates to a hybridoma which produces the above-described monoclonal antibody, or binding fragment thereof. A hybridoma is an immortalized cell line which is capable of secreting a specific monoclonal antibody.

In general, techniques for preparing monoclonal antibodies and hybridomas are well known in the art (Campbell, “Monoclonal Antibody Technology: Laboratory Techniques in Biochemistry and Molecular Biology,” Elsevier Science Publishers, Amsterdam, The Netherlands, 1984; St. Groth et al., J. Immunol. Methods 35:1-21, 1980). Any animal (mouse, rabbit, and the like) which is known to produce antibodies can be immunized with the selected polypeptide. Methods for immunization are well known in the art. Such methods include subcutaneous or intraperitoneal injection of the polypeptide. One skilled in the art will recognize that the amount of polypeptide used for immunization will vary based on the animal which is immunized, the antigenicity of the polypeptide and the site of injection.

The polypeptide may be modified or administered in an adjuvant in order to increase the peptide antigenicity. Methods of increasing the antigenicity of a polypeptide are well known in the art. Such procedures include coupling the antigen with a heterologous protein (such as globulin or galactosidase) or through the inclusion of an adjuvant during immunization.

For monoclonal antibodies, spleen cells from the immunized animals are removed, fused with myeloma cells, such as SP2/0-Agl4 myeloma cells, and allowed to become monoclonal antibody producing hybridoma cells. Any one of a number of methods well known in the art can be used to identify the hybridoma cell which produces an antibody with the desired characteristics. These include screening the hybridomas with an ELISA assay, western blot analysis, or radioimmunoassay (Lutz et al., Exp. Cell Res. 175:109-124, 1988). Hybridomas secreting the desired antibodies are cloned and the class and subclass are determined using procedures known in the art (Campbell, “Monoclonal Antibody Technology: Laboratory Techniques in Biochemistry and Molecular Biology”, supra, 1984).

For polyclonal antibodies, antibody-containing antisera is isolated from the immunized animal and is screened for the presence of antibodies with the desired specificity using one of the above-described procedures. The above-described antibodies may be detectably labeled. Antibodies can be detectably labeled through the use of radioisotopes, affinity labels (such as biotin, avidin, and the like), enzymatic labels (such as horseradish peroxidase, alkaline phosphatase, and the like) fluorescent labels (such as FITC or rhodamine, and the like), paramagnetic atoms, and the like. Procedures for accomplishing such labeling are well-known in the art, for example, see Stemberger et al., J. Histochem. Cytochem. 18:315, 1970; Bayer et al., Meth. Enzym. 62:308, 1979; Engval et al., Immunol. 109:129, 1972; Goding, J. Immunol. Meth. 13:215, 1976. The labeled antibodies of the present invention can be used for in vitro, in vivo, and in situ assays to identify cells or tissues which express a specific peptide.

The above-described.antibodies may also be immobilized on a solid support. Examples of such solid supports include plastics such as polycarbonate, complex carbohydrates such as agarose and sepharose, acrylic resins such as polyacrylamide and latex beads. Techniques for coupling antibodies to such solid supports are well known in the art (Weir et al., “Handbook of Experimental Immunology” 4th Ed., Blackwell Scientific Publications, Oxford, England, Chapter 10, 1986; Jacoby et al., Meth. Enzym. 34, Academic Press, N.Y., 1974). The immobilized antibodies of the present invention can be used for in vitro, in vivo, and in situ assays as well as in immunochromotography.

Furthermore, one skilled in the art can readily adapt currently available procedures, as well as the techniques, methods and kits disclosed herein with regard to antibodies, to generate peptides capable of binding to a specific peptide sequence in order to generate rationally designed antipeptide peptides (Hurby et al., “Application of Synthetic Peptides: Antisense Peptides”, In Synthetic Peptides, A User's Guide, W. H. Freeman, NY, pp. 289-307, 1992; Kaspczak et al., Biochemistry 28:9230-9238, 1989).

Anti-peptide peptides can be generated by replacing the basic amino acid residues found in the peptide sequences of the kinases of the invention with acidic residues, while maintaining hydrophobic and uncharged polar groups. For example, lysine, arginine, and/or histidine residues are replaced with aspartic acid or glutamic acid and glutamic acid residues are replaced by lysine, arginine or histidine.

The present invention also encompasses a method of detecting a kinase polypeptide in a sample, comprising: (a) contacting the sample with an above-described antibody, under conditions such that immunocomplexes form, and (b) detecting the presence of said antibody bound to the polypeptide. In detail, the methods comprise incubating a test sample with one or more of the antibodies of the present invention and assaying whether the antibody binds to the test sample. Altered levels of a kinase of the invention in a sample as compared to normal levels may indicate disease.

Conditions for incubating an antibody with a test sample vary. Incubation conditions depend on the format employed in the assay, the detection methods employed, and the type and nature of the antibody used in the assay. One skilled in the art will recognize that any one of the commonly available immunological assay formats (such as radioimmunoassays, enzyme-linked immunosorbent assays, diffusion-based Ouchterlony, or rocket immunofluorescent assays) can readily be adapted to employ the antibodies of the present invention. Examples of such assays can be found in Chard (“An Introduction to Radioimmunoassay and Related Techniques” Elsevier Science Publishers, Amsterdam, The Netherlands, 1986), Bullock et al. (“Techniques in Immunocytochemistry,” Academic Press, Orlando, Fla. Vol. 1, 1982; Vol. 2, 1983; Vol. 3, 1985), Tijssen (“Practice and Theory of Enzyme Immunoassays: Laboratory Techniques in Biochemistry and Molecular Biology,” Elsevier Science Publishers, Amsterdam, The Netherlands, 1985).

The immunological assay test samples of the present invention include cells, protein or membrane extracts of cells, or biological fluids such as blood, serum, plasma, or urine. The test samples used in the above-described method will vary based on the assay format, nature of the detection method and the tissues, cells or extracts used as the sample to be assayed. Methods for preparing protein extracts or membrane extracts of cells are well known in the art and can readily be adapted in order to obtain a sample which is testable with the system utilized.

A kit contains all the necessary reagents to carry out the previously described methods of detection. The kit may comprise: (i) a first container means containing an above-described antibody, and (ii) second container means containing a conjugate comprising a binding partner of the antibody and a label. In another preferred embodiment, the kit further comprises one or more other containers comprising one or more of the following: wash reagents and reagents capable of detecting the presence of bound antibodies.

Examples of detection reagents include, but are not limited to, labeled secondary antibodies, or in the alternative, if the primary antibody is labeled, the chromophoric, enzymatic, or antibody binding reagents which are capable of reacting with the labeled antibody. The compartmentalized kit may be as described above for nucleic acid probe kits. One skilled in the art will readily recognize that the antibodies described in the present invention can readily be incorporated into one of the established kit formats which are well known in the art.

Isolation of Compounds Capable of Interacting with Kinases

The present invention also relates to a method of detecting a compound capable of binding to a kinase of the invention comprising incubating the compound with a kinase of the invention and detecting the presence of the compound bound to the kinase. The compound may be present within a complex mixture, for example, serum, body fluid, or cell extracts.

The present invention also relates to a method of detecting an agonist or antagonist of kinase activity or kinase binding partner activity comprising incubating cells that produce a kinase of the invention in the presence of a compound and detecting changes in the level of kinase activity or kinase binding partner activity. The compounds thus identified would produce a change in activity indicative of the presence of the compound. The compound may be present within a complex mixture, for example, serum, body fluid, or cell extracts. Once the compound is identified it can be isolated using techniques well known in the art.

Modulating Polypeptide Activity:

The invention additionally provides methods for treating a disease or abnormal condition by administering to a patient in need of such treatment a substance that modulates the activity of a polypeptide selected from the group consisting of SEQ ID NO:1-87. Preferably, the disease is selected from the group consisting of rheumatoid arthritis, atherosclerosis, autoimmune disorders, organ transplantation, myocardial infarction, cardiomyopathies, stroke, renal failure, oxidative stress-related neurodegenerative disorders, viral and bacterial infections, metabolic and reproductive disorders, and cancer.

Substances useful for treatment of disorders or diseases preferably show positive results in one or more assays for an activity corresponding to treatment of the disease or disorder in question. Substances that modulate the activity of the polypeptides preferably include, but are not limited to, antisense oligonucleotides, ribosymes, RNAi, and inhibitors of protein kinases.

The term “preventing” refers to decreasing the probability that an organism contracts or develops an abnormal condition.

The present invention also encompasses a method of agonizing (stimulating) or antagonizing kinase associated activity in a mammal comprising administering to said mammal an agonist or antagonist to a kinase of the invention in an amount sufficient to effect said agonism or antagonism. A method of treating diseases in a mammal with an agonist or antagonist of the activity of one of the kinases of the invention comprising administering the agonist or antagonist to a mammal in an amount sufficient to agonize or antagonize kinase-associated functions is also encompassed in the present application.

In an effort to discover novel treatments for diseases, biomedical researchers and chemists have designed, synthesized, and tested molecules that inhibit the function of protein kinases. Some small organic molecules form a class of compounds that modulate the function of protein kinases. Examples of molecules that have been reported to inhibit the function of protein kinases include, but are not limited to, bis monocyclic, bicyclic or heterocyclic aryl compounds (PCT WO 92/20642, published Nov. 26, 1992 by Maguire et al.), vinylene-azaindole derivatives (PCT WO 94/14808, published Jul. 7, 1994 by Ballinari et al.), 1-cyclopropyl-4-pyridyl-quinolones (U.S. Pat. No. 5,330,992), styryl compounds (U.S. Pat. No. 5,217,999), styryl-substituted pyridyl compounds (U.S. Pat. No. 5,302,606), certain quinazoline derivatives (EP Application No. 0 566 266 A1), seleoindoles and selenides (PCT WO 94/03427, published Feb. 17, 1994 by Denny et al.), tricyclic polyhydroxylic compounds (PCT WO 92/21660, published Dec. 10, 1992 by Dow), and benzylphosphonic acid compounds (PCT WO 91/15495, published Oct. 17, 1991 by Dow et al).

Compounds that can traverse cell membranes and are resistant to acid hydrolysis are potentially advantageous as therapeutics as they can become highly bioavailable after being administered orally to patients. However, many of these protein kinase inhibitors only weakly inhibit the function of protein kinases. In addition, many inhibit a variety of protein kinases and will therefore cause multiple side-effects as therapeutics for diseases.

Some indolinone compounds, however, form classes of acid resistant and membrane permeable organic molecules. WO 96/22976 (published Aug. 1, 1996 by Ballinari et al.) describes hydrosoluble indolinone compounds that harbor tetralin, naphthalene, quinoline, and indole substituents fused to the oxindole ring. These bicyclic substituents are in turn substituted with polar moieties including hydroxylated alkyl, phosphate, and ether moieties. U.S. patent application Ser. No. 08/702,232, filed Aug. 23, 1996, entitled “Indolinone Combinatorial Libraries and Related Products and Methods for the Treatment of Disease” by Tang et al. (corresponding to WO 98/07695) and 08/485,323, filed Jun. 7, 1995, entitled “Benzylidene-Z-Indoline Compounds for the Treatment of Disease” by Tang et al. (corresponding to U.S. Pat. No. 5,880,141) and International Patent Publications WO 96/40116, published Dec. 19, 1996 by Tang, et al. (see also U.S. Pat. Nos. 5,792,783, 5,883,116, 5883,113, 6,469,032, 6,225,335, 5,886,020), and WO 96/22976, published Aug. 1, 1996 by Ballinari et al., all of which are incorporated herein by reference in their entirety, including any drawings, figures, or tables, describe indolinone chemical libraries of indolinone compounds harboring other bicyclic moieties as well as monocyclic moieties fused to the oxindole ring. Applications 08/702,232, filed Aug. 23, 1996, entitled “Indolinone Combinatorial Libraries and Related Products and Methods for the Treatment of Disease” by Tang et al. (corresponding to WO 98/07595), 08/485,323, filed Jun. 7, 1995, entitled “Benzylidene-Z-Indoline Compounds for the Treatment of Disease” by Tang et al. (corresponding to U.S. Pat. No. 5,880,141), and WO 96/22976, published Aug. 1, 1996 by Ballinari et al. teach methods of indolinone synthesis, methods of testing the biological activity of indolinone compounds in cells, and inhibition patterns of indolinone derivatives.

Other examples of substances capable of modulating kinase activity include, but are not limited to, tyrphostins, quinazolines, quinoxolines, and quinolines. The quinazolines, tyrphostins, quinolines, and quinoxolines referred to above include well known compounds such as those described in the literature. For example, representative publications describing quinazolines include Barker et al., EPO Publication No. 0 520 722 A1; Jones et al., U.S. Pat. No. 4,447,608; Kabbe et al., U.S. Pat. No. 4,757,072; Kaul and Vougioukas, U.S. Pat. No. 5,316,553; Kreighbaum and Corner, U.S. Pat. No. 4,343,940; Pegg and Wardleworth, EPO Publication No. 0 562 734 A1; Barker et al., (1991) Proc. of Am. Assoc. for Cancer Research 32:327; Bertino, J. R., (1979) Cancer Research 3:293-304; Bertino, J. R., (1979) Cancer Research 9(2 part 1):293-304; Curtin et al., (1986) Br. J. Cancer 53:361-368; Fernandes et al., (1983) Cancer Research 43:1117-1123; Ferris et al. J. Org. Chem. 44(2):173-178; Fry et al., (1994) Science 265:1093-1095; Jackman et al., (1981) Cancer Research 51:5579-5586; Jones et al. J. Med. Chem. 29(6):1114-1118; Lee and Skibo, (1987) Biochemistry 26(23):7355-7362; Lemus et al., (1989) J. Org. Chem. 54:3511-3518; Ley and Seng, (1975) Synthesis 1975:415-522; Maxwell et al., (1991) Magnetic Resonance in Medicine 17:189-196; Mini et al., (1985) Cancer Research 45:325-330; Phillips and Castle, J. (1980) Heterocyclic Chem. 17(19):1489-1596; Reece et al., (1977) Cancer Research 47(11):2996-2999; Sculier et al., (1986) Cancer Immunol. and Immunother. 23, A65; Sikora et al., (1984) Cancer Letters 23:289-295; Sikora et al., (1988) Analytical Biochem. 172:344-355; all of which are incorporated herein by reference in their entirety, including any drawings.

Quinoxaline is described in Kaul and Vougioukas, U.S. Pat. No. 5,316,553, incorporated herein by reference in its entirety, including any drawings.

Quinolines are described in Dolle et al., (1994) J. Med. Chem. 37:2627-2629; MaGuire, J. (1994) Med. Chem. 37:2129-2131; Burke et al., (1993) J. Med. Chem. 36:425-432; and Burke et al. (1992) BioOrganic Med. Chem. Letters 2:1771-1774, all of which are incorporated by reference in their entirety, including any drawings.

Tyrphostins are described in Allen et al., (1993) Clin. Exp. Immunol. 91:141-156; Anafi et al., (1993) Blood 82:12, 3524-3529; Baker et al., (1992) J. Cell Sci. 102:543-555; Bilder et al., (1991) Amer. Physiol. Soc. pp. 6363-6143:C721-C730; Brunton et al., (1992) Proceedings of Amer. Assoc. Cancer Rsch. 33:558; Bryckaert et al., (1992) Exp. Cell Research 199:255-261; Dong et al., (1993) J. Leukocyte Biology 53:53-60; Dong et al., (1993) J. Immunol. 151(5):2717-2724; Gazit et al., (1989) J. Med. Chem. 32, 2344-2352; Gazit et al., (1993) J. Med. Chem. 36:3556-3564; Kaur et al., (1994) Anti-Cancer Drugs 5:213-222; King et al., (1991) Biochem. J. 275:413-418; Kuo et al., (1993) Cancer Letters 74:197-202; Levitzki, A., (1992) The FASEB J. 6:3275-3282; Lyall et al., (1989) J. Biol. Chem. 264:14503-14509; Peterson et al., (1993) The Prostate 22:335-345; Pillemer et al., (1992) Int. J. Cancer 50:80-85; Posner et al., (1993) Molecular Pharmacology 45:673-683; Rendu et al., (1992) Biol. Pharmacology 44(5):881-888; Sauro and Thomas, (1993) Life Sciences 53:371-376; Sauro and Thomas, (1993) J. Pharm. and Experimental Therapeutics 267(3):119-1125; Wolbring et al., (1994) J. Biol. Chem. 269(36):22470-22472; and Yoneda et al., (1991) Cancer Research 51:4430-4435; all of which are incorporated herein by reference in their entirety, including any drawings.

DNA Constructs Comprising a Kinase Nucleic Acid Molecule and Cells Containing These Constructs:

The present invention also relates to a recombinant DNA molecule comprising, 5′ to 3′, a promoter effective to initiate transcription in a host cell and the above-described nucleic acid molecules. In addition, the present invention relates to a recombinant DNA molecule comprising a vector and an above-described nucleic acid molecule. The present invention also relates to a nucleic acid molecule comprising a transcriptional region functional in a cell, a sequence complementary to an RNA sequence encoding an amino acid sequence corresponding to the above-described polypeptide, and a transcriptional termination region functional in said cell. The above-described molecules may be isolated and/or purified DNA molecules.

The present invention also relates to a cell or organism that contains an above-described nucleic acid molecule and thereby is capable of expressing a polypeptide. The polypeptide may be purified from cells which have been altered to express the polypeptide. A cell is said to be “altered to express a desired polypeptide” when the cell, through genetic manipulation, is made to produce a protein which it normally does not produce or which the cell normally produces at lower levels. One skilled in the art can readily adapt procedures for introducing and expressing either genomic, cDNA, or synthetic sequences into either eukaryotic or prokaryotic cells.

A nucleic acid molecule, such as DNA, is said to be “capable of expressing” a polypeptide if it contains nucleotide sequences which contain transcriptional and translational regulatory information and such sequences are “operably linked” to nucleotide sequences which encode the polypeptide. An operable linkage is a linkage in which the regulatory DNA sequences and the DNA sequence sought to be expressed are connected in such a way as to permit gene sequence expression. The precise nature of the regulatory regions needed for gene sequence expression may vary from organism to organism, but shall in general include a promoter region which, in prokaryotes, contains both the promoter (which directs the initiation of RNA transcription) as well as the DNA sequences which, when transcribed into RNA, will signal synthesis initiation. Such regions will normally include those 5′-non-coding sequences involved with initiation of transcription and translation, such as the TATA box, capping sequence, CAAT sequence, and the like.

If desired, the non-coding region 3′ to the sequence encoding a kinase of the invention may be obtained by the above-described methods. This region may be retained for its transcriptional termination regulatory sequences, such as termination and polyadenylation. Thus, by retaining the 3′-region naturally contiguous to the DNA sequence encoding a kinase of the invention, the transcriptional termination signals may be provided. Where the transcriptional termination signals are not satisfactorily functional in the expression host cell, then a 3′ region functional in the host cell may be substituted.

Two DNA sequences (such as a promoter region sequence and a sequence encoding a kinase of the invention) are said to be operably linked if the nature of the linkage between the two DNA sequences does not (1) result in the introduction of a frame-shift mutation, (2) interfere with the ability of the promoter region sequence to direct the transcription of a gene sequence encoding a kinase of the invention, or (3) interfere with the ability of the gene sequence of a kinase of the invention to be transcribed by the promoter region sequence. Thus, a promoter region would be operably linked to a DNA sequence if the promoter were capable of effecting transcription of that DNA sequence. Thus, to express a gene encoding a kinase of the invention, transcriptional and translational signals recognized by an appropriate host are necessary.

The present invention encompasses the expression of a gene encoding a kinase of the invention (or a functional derivative thereof) in either prokaryotic or eukaryotic cells. Prokaryotic hosts are, generally, very efficient and convenient for the production of recombinant proteins and are, therefore, one type of preferred expression system for kinases of the invention. Prokaryotes most frequently are represented by various strains of E. coli. However, other microbial strains may also be used, including other bacterial strains.

In prokaryotic systems, plasmid vectors that contain replication sites and control sequences derived from a species compatible with the host may be used. Examples of suitable plasmid vectors may include pBR322, pUC118, pUC119 and the like; suitable phage or bacteriophage vectors may include λgt10, λgt11 and the like; and suitable virus vectors may include pMAM-neo, pKRC and the like. Preferably, the selected vector of the present invention has the capacity to replicate in the selected host cell.

Recognized prokaryotic hosts include bacteria such as E. coli, Bacillus, Streptomyces, Pseudomonas, Salmonella, Serratia, and the like. However, under such conditions, the polypeptide will not be glycosylated. The prokaryotic host must be compatible with the replicon and control sequences in the expression plasmid.

To express a kinase of the invention (or a functional derivative thereof) in a prokaryotic cell, it is necessary to operably link the sequence encoding the kinase of the invention to a functional prokaryotic promoter. Such promoters may be either constitutive or, more preferably, regulatable (i.e., inducible or derepressible). Examples of constitutive promoters include the int promoter of bacteriophage λ, the bla promoter of the β-lactamase gene sequence of pBR322, and the cat promoter of the chloramphenicol acetyl transferase gene sequence of pPR325, and the like. Examples of inducible prokaryotic promoters include the major right and left promoters of bacteriophage λ (P_{L} and P_{R}), the trp, λrecA, acZ, λacI, and gal promoters of E. coli, the α-amylase (Ulmanen et al., J. Bacteriol. 162:176-182, 1985) and the q-28-specific promoters of B. subtilis (Gilman et al., Gene Sequence 32:11-20, 1984), the promoters of the bacteriophages of Bacillus (Gryczan, in: The Molecular Biology of the Bacilli, Academic Press, Inc., NY, 1982), and Streptomyces promoters (Ward et al., Mol. Gen. Genet. 203:468-478, 1986). Prokaryotic promoters are reviewed by Glick (Ind. Microbiot. 1:277-282, 1987), Cenatiempo (Biochimie 68:505-516, 1986), and Gottesman (Ann. Rev. Genet. 18:415-442, 1984).

Proper expression in a prokaryotic cell also requires the presence of a ribosome-binding site upstream of the gene sequence-encoding sequence. Such ribosome-binding sites are disclosed, for example, by Gold et al. (Ann. Rev. Microbiol. 35:365-404, 1981). The selection of control sequences, expression vectors, transformation methods, and the like, are dependent on the type of host cell used to express the gene. As used herein, “cell”, “cell line”, and “cell culture” may be used interchangeably and all such designations include progeny. Thus, the words “transformants” or “transformed cells” include the primary subject cell and cultures derived therefrom, without regard to the number of transfers. It is also understood that all progeny may not be precisely identical in DNA content, due to deliberate or inadvertent mutations. However, as defined, mutant progeny have the same functionality as that of the originally transformed cell.

Host cells which may be used in the expression systems of the present invention are not strictly limited, provided that they are suitable for use in the expression of the kinase polypeptide of interest. Suitable hosts may often include eukaryotic cells. Preferred eukaryotic hosts include, for example, yeast, fungi, insect cells, mammalian cells either in vivo, or in tissue culture. Mammalian cells which may be useful as hosts include HeLa cells, cells of fibroblast origin such as VERO or CHO-K1, or cells of lymphoid origin and their derivatives. Preferred mammalian host cells include SP2/0 and J558L, as well as neuroblastoma cell lines such as IMR 332, which may provide better capacities for correct post-translational processing.

In addition, plant cells are also available as hosts, and control sequences compatible with plant cells are available, such as the cauliflower mosaic virus 35S and 19S, and nopaline synthase promoter and polyadenylation signal sequences. Another preferred host is an insect cell, for example the Drosophila larvae. Using insect cells as hosts, the Drosophila alcohol dehydrogenase promoter can be used (Rubin, Science 240:1453-1459, 1988). Alternatively, baculovirus vectors can be engineered to express large amounts of kinases of the invention in insect cells (Jasny, Science 238:1653, 1987; Miller et al., in: Genetic Engineering, Vol. 8, Plenum, Setlow et al., eds., pp. 277-297, 1986).

Any of a series of yeast expression systems can be utilized which incorporate promoter and termination elements from the actively expressed sequences coding for glycolytic enzymes that are produced in large quantities when yeast are grown in mediums rich in glucose. Known glycolytic gene sequences can also provide very efficient transcriptional control signals. Yeast provides substantial advantages in that it can also carry out post-translational modifications. A number of recombinant DNA strategies exist utilizing strong promoter sequences and high copy number plasmids which can be utilized for production of the desired proteins in yeast. Yeast recognizes leader sequences on cloned mammalian genes and secretes peptides bearing leader sequences (i.e., pre-peptides). Several possible vector systems are available for the expression of kinases of the invention in a mammalian host.

A wide variety of transcriptional and translational regulatory sequences may be employed, depending upon the nature of the host. The transcriptional and translational regulatory signals may be derived from viral sources, such as adenovirus, bovine papilloma virus, cytomegalovirus, simian virus, or the like, where the regulatory signals are associated with a particular gene sequence which has a high level of expression. Alternatively, promoters from mammalian expression products, such as actin, collagen, myosin, and the like, may be employed. Transcriptional initiation regulatory signals may be selected which allow for repression or activation, so that expression of the gene sequences can be modulated. Of interest are regulatory signals which are temperature-sensitive so that by varying the temperature, expression can be repressed or initiated, or are subject to chemical (such as metabolite) regulation.

Expression of kinases of the invention in eukaryotic hosts requires the use of eukaryotic regulatory regions. Such regions will, in general, include a promoter region sufficient to direct the initiation of RNA synthesis. Preferred eukaryotic promoters include, for example, the promoter of the mouse metallothionein I gene sequence (Hamer et al., J. Mol. Appl. Gen. 1:273-288, 1982); the TK promoter of Herpes virus (McKnight, Cell 31:355-365, 1982); the SV40 early promoter (Benoist et al., Nature (London) 290:304-31, 1981); and the yeast gal4 gene sequence promoter (Johnston et al., Proc. Natl. Acad. Sci. (USA) 79:6971-6975, 1982; Silver et al., Proc. Natl. Acad. Sci. (USA) 81:5951-5955, 1984).

Translation of eukaryotic mRNA is initiated at the codon which encodes the first methionine. For this reason, it is preferable to ensure that the linkage between a eukaryotic promoter and a DNA sequence which encodes a kinase of the invention (or a functional derivative thereof) does not contain any intervening codons which are capable of encoding a methionine (i.e., AUG). The presence of such codons results either in the formation of a fusion protein (if the AUG codon is in the same reading frame as the kinase of the invention coding sequence) or a frame-shift mutation (if the AUG codon is not in the same reading frame as the kinase of the invention coding sequence).

A nucleic acid molecule encoding a kinase of the invention and an operably linked promoter may be introduced into a recipient prokaryotic or eukaryotic cell either as a nonreplicating DNA or RNA molecule, which may either be a linear molecule or, more preferably, a closed covalent circular molecule. Since such molecules are incapable of autonomous replication, the expression of the gene may occur through the transient expression of the introduced sequence. Alternatively, permanent expression may occur through the integration of the introduced DNA sequence into the host chromosome.

A vector may be employed which is capable of integrating the desired gene sequences into the host cell chromosome. Cells which have stably integrated the introduced DNA into their chromosomes can be selected by also introducing one or more markers which allow for selection of host cells which contain the expression vector. The marker may provide for prototrophy to an auxotrophic host, biocide resistance, e.g., antibiotics, or heavy metals, such as copper, or the like. The selectable marker gene sequence can either be directly linked to the DNA gene sequences to be expressed, or introduced into the same cell by co-transfection. Additional elements may also be needed for optimal synthesis of mRNA. These elements may include splice signals, as well as transcription promoters, enhancers, and termination signals. cDNA expression vectors incorporating such elements include those described by Okayama (Mol. Cell. Biol. 3:280-289, 1983).

The introduced nucleic acid molecule can be incorporated into a plasmid or viral vector capable of autonomous replication in the recipient host. Any of a wide variety of vectors may be employed for this purpose. Factors of importance in selecting a particular plasmid or viral vector include: the ease with which recipient cells that contain the vector may be recognized and selected from those recipient cells which do not contain the vector; the number of copies of the vector which are desired in a particular host; and whether it is desirable to be able to “shuttle” the vector between host cells of different species.

Preferred prokaryotic vectors include plasmids such as those capable of replication in E. coli (such as, for example, pBR322, ColEl, pSC101, PACYC 184, πVX; “Molecular Cloning: A Laboratory Manual”, 1989, supra). Bacillus plasmids include pC194, pC221, pT127, and the like (Gryczan, In: The Molecular Biology of the Bacilli, Academic Press, NY, pp. 307-329, 1982). Suitable Streptomyces plasmids include p1J101 (Kendall et al., J. Bacteriol. 169:4177-4183, 1987), and streptomyces bacteriophages such as φC31 (Chater et al., In: Sixth International Symposium on Actinomycetales Biology, Akademiai Kaido, Budapest, Hungary, pp. 45-54, 1986). Pseudomonas plasmids are reviewed by John et al. (Rev. Infect. Dis. 8:693-704, 1986), and Izaki (Jpn. J. Bacteriol. 33:729-742, 1978).

Preferred eukaryotic plasmids include, for example, BPV, vaccinia, SV40, 2-micron circle, and the like, or their derivatives. Such plasmids are well known in the art (Botstein et al., Miami Wntr. Symp. 19:265-274, 1982; Broach, In: “The Molecular Biology of the Yeast Saccharomyces: Life Cycle and Inheritance”, Cold Spring Harbor Laboratory, Cold Spring Harbor, N.Y., p. 445-470, 1981; Broach, Cell 28:203-204, 1982; Bollon et al., J. Clin. Hematol. Oncol. 10:39-48, 1980; Maniatis, In: Cell Biology: A Comprehensive Treatise, Vol. 3, Gene Sequence Expression, Academic Press, NY, pp. 563-608, 1980).

Once the vector or nucleic acid molecule containing the construct(s) has been prepared for expression, the DNA construct(s) may be introduced into an appropriate host cell by any of a variety of suitable means, i.e., transformation, transfection, conjugation, protoplast fusion, electroporation, particle gun technology, calcium phosphate-precipitation, direct microinjection, and the like. After the introduction of the vector, recipient cells are grown in a selective medium, which selects for the growth of vector-containing cells. Expression of the cloned gene(s) results in the production of a kinase of the invention, or fragments thereof. This can take place in the transformed cells as such, or following the induction of these cells to differentiate (for example, by administration of bromodeoxyuracil to neuroblastoma cells or the like). A variety of incubation conditions can be used to form the peptide of the present invention. The most preferred conditions are those which mimic physiological conditions.

Transgenic Animals:

A variety of methods are available for the production of transgenic animals associated with this invention. DNA can be injected into the pronucleus of a fertilized egg before fusion of the male and female pronuclei, or injected into the nucleus of an embryonic cell (e.g., the nucleus of a two-cell embryo) following the initiation of cell division (Brinster et al., Proc. Nat. Acad. Sci. USA 82:4438-4442, 1985). Embryos can be infected with viruses, especially retroviruses, modified to carry inorganic-ion receptor nucleotide sequences of the invention.

Pluripotent stem cells derived from the inner cell mass of the embryo and stabilized in culture can be manipulated in culture to incorporate nucleotide sequences of the invention. A transgenic animal can be produced from such cells through implantation into a blastocyst that is implanted into a foster mother and allowed to come to term. Animals suitable for transgenic experiments can be obtained from standard commercial sources such as Charles River (Wilmington, Mass.), Taconic (Germantown, N.Y.), Harlan Sprague Dawley (Indianapolis, Ind.), etc.

The procedures for manipulation of the rodent embryo and for microinjection of DNA into the pronucleus of the zygote are well known to those of ordinary skill in the art (Hogan et al., supra). Microinjection procedures for fish, amphibian eggs and birds are detailed in Houdebine and Chourrout (Experientia 47:897-905, 1991). Other procedures for introduction of DNA into tissues of animals are described in U.S. Pat. No. 4,945,050 (Sanford et al., Jul. 30, 1990).

By way of example only, to prepare a transgenic mouse, female mice are induced to superovulate. Females are placed with males, and the mated females are sacrificed by CO₂ asphyxiation or cervical dislocation and embryos are recovered from excised oviducts. Surrounding cumulus cells are removed. Pronuclear embryos are then washed and stored until the time of injection. Randomly cycling adult female mice are paired with vasectomized males. Recipient females are mated at the same time as donor females. Embryos then are transferred surgically. The procedure for generating transgenic rats is similar to that of mice (Hammer et al., Cell 63:1099-1112, 1990).

Methods for the culturing of embryonic stem (ES) cells and the subsequent production of transgenic animals by the introduction of DNA into ES cells using methods such as electroporation, calcium phosphate/DNA precipitation and direct injection also are well known to those of ordinary skill in the art (Teratocarcinomas and Embryonic Stem Cells, A Practical Approach, E. J. Robertson, ed., IRL Press, 1987).

In cases involving random gene integration, a clone containing the sequence(s) of the invention is co-transfected with a gene encoding resistance. Alternatively, the gene encoding neomycin resistance is physically linked to the sequence(s) of the invention. Transfection and isolation of desired clones are carried out by any one of several methods well known to those of ordinary skill in the art (E. J. Robertson, supra).

DNA molecules introduced into ES cells can also be integrated into the chromosome through the process of homologous recombina-tion (Capecchi, Science 244:1288-1292, 1989). Methods for positive selection of the recombination event (i.e., neo resistance) and dual positive-negative selection (i.e., neo resistance and gancyclovir resistance) and the subsequent identification of the desired clones by PCR have been described by Capecchi, supra and Joyner et al. (Nature 338:153-156, 1989), the teachings of which are incorporated herein in their entirety including any drawings. The final phase of the procedure is to inject targeted ES cells into blastocysts and to transfer the blastocysts into pseudopregnant females. The resulting chimeric animals are bred and the offspring are analyzed by Southern blotting to identify individuals that carry the transgene. Procedures for the production of non-rodent mammals and other animals have been discussed by others (Houdebine and Chourrout, supra; Pursel et al., Science 244:1281-1288, 1989; and Simms et al., Bio/Technology 6:179-183, 1988).

Thus, the invention provides transgenic, nonhuman mammals containing a transgene encoding a kinase of the invention or a gene affecting the expression of the kinase. Such transgenic nonhuman mammals are particularly useful as an in vivo test system for studying the effects of introduction of a kinase, or regulating the expression of a kinase (i.e., through the introduction of additional genes, antisense nucleic acids, or ribozymes).

A “transgenic animal” is an animal having cells that contain DNA which has been artificially inserted into a cell, which DNA becomes part of the genome of the animal which develops from that cell. Preferred transgenic animals are primates, mice, rats, cows, pigs, horses, goats, sheep, dogs and cats. The transgenic DNA may encode human kinases. Native expression in an animal may be reduced by providing an amount of antisense RNA or DNA effective to reduce expression of the receptor.

Gene Therapy:

Kinases or their genetic sequences will also be useful in gene therapy (reviewed in Miller, Nature 357:455-460, 1992). Miller states that advances have resulted in practical approaches to human gene therapy that have demonstrated positive initial results. The basic science of gene therapy is described in Mulligan (Science 260:926-931, 1993).

In one preferred embodiment, an expression vector containing a kinase coding sequence is inserted into cells, the cells are grown in vitro and then infused in large numbers into patients. In another preferred embodiment, a DNA segment containing a promoter of choice (for example a strong promoter) is transferred into cells containing an endogenous gene encoding kinases of the invention in such a manner that the promoter segment enhances expression of the endogenous kinase gene (for example, the promoter segment is transferred to the cell such that it becomes directly linked to the endogenous kinase gene).

The gene therapy may involve the use of an adenovirus containing kinase cDNA targeted to a tumor, systemic kinase increase by implantation of engineered cells, injection with kinase-encoding virus, or injection of naked kinase DNA into appropriate tissues.

Target cell populations may be modified by introducing altered forms of one or more components of the protein complexes in order to modulate the activity of such complexes. For example, by reducing or inhibiting a complex component activity within target cells, an abnormal signal transduction event(s) leading to a condition may be decreased, inhibited, or reversed. Deletion or missense mutants of a component, that retain the ability to interact with other components of the protein complexes but cannot function in signal transduction, may be used to inhibit an abnormal, deleterious signal transduction event.

Expression vectors derived from viruses such as retroviruses, vaccinia virus, adenovirus, adeno-associ-ated virus, herpes viruses, several RNA viruses, or bovine papilloma virus, may be used for delivery of nucleotide sequences (e.g., cDNA) encoding recombinant kinase of the invention protein into the targeted cell population (e.g., tumor cells). Methods which are well known to those skilled in the art can be used to construct recombinant viral vectors containing coding sequences (Maniatis et al., Molecular Cloning: A Laboratory Manual, Cold Spring Harbor Laboratory, N.Y., 1989; Ausubel et al., Current Proto-cols in Molecular Biology, Greene Publishing Associates and Wiley Interscience, N.Y., 1989). Alternatively, recombinant nucleic acid molecules encoding protein sequences can be used as naked DNA or in a reconstituted system e.g., liposomes or other lipid systems for delivery to target cells (e.g., Felgner et al., Nature 337:387-8, 1989). Several other methods for the direct transfer of plasmid DNA into cells exist for use in human gene therapy and involve targeting the DNA to receptors on cells by complexing the plasmid DNA to proteins.

In its simplest form, gene transfer can be performed by simply injecting minute amounts of DNA into the nucleus of a cell, through a process of microinjection (Capecchi, Cell 22:479-88, 1980). Once recombinant genes are introduced into a cell, they can be recognized by the cell's normal mechanisms for transcription and translation, and a gene product will be expressed. Other methods have also been attempted for introducing DNA into larger numbers of cells. These methods include: transfection, wherein DNA is precipitated with calcium phosphate and taken into cells by pinocytosis (Chen et al., Mol. Cell Biol. 7:2745-52, 1987); electroporation, wherein cells are exposed to large voltage pulses to introduce holes into the membrane (Chu et al., Nucleic Acids Res. 15:1311-26, 1987); lipofection/liposome fusion, wherein DNA is packaged into lipophilic vesicles which fuse with a target cell (Felgner et al., Proc. Natl. Acad. Sci. USA. 84:7413-7417, 1987); and particle bombardment using DNA bound to small projectiles (Yang et al., Proc. Natl. Acad. Sci. 87:9568-9572, 1990). Another method for introducing DNA into cells is to couple the DNA to chemically modified proteins.

It has also been shown that adenovirus proteins are capable of destabilizing endosomes and enhancing the uptake of DNA into cells. The admixture of adenovirus to solutions containing DNA complexes, or the binding of DNA to polylysine covalently attached to adenovirus using protein crosslinking agents substantially improves the uptake and expression of the recombinant gene (Curiel et al., Am. J. Respir. Cell. Mol. Biol., 6:247-52, 1992).

As used herein “gene transfer” means the process of introducing a foreign nucleic acid molecule into a cell. Gene transfer is commonly performed to enable the expres-sion of a particular product encoded by the gene. The product may include a protein, polypeptide, anti-sense DNA or RNA, or enzymatically active RNA. Gene transfer can be performed in cultured cells or by direct administration into animals. Generally gene transfer involves the process of nucleic acid contact with a target cell by non-specific or receptor mediated interactions, uptake of nucleic acid into the cell through the membrane or by endocytosis, and release of nucleic acid into the cyto-plasm from the plasma membrane or endosome. Expression may require, in addition, movement of the nucleic acid into the nucleus of the cell and binding to appropriate nuclear factors for transcription.

As used herein “gene therapy” is a form of gene transfer and is included within the definition of gene transfer as used herein and specifically refers to gene transfer to express a therapeutic product from a cell in vivo or in vitro. Gene transfer can be performed ex vivo on cells which are then transplanted into a patient, or can be performed by direct administration of the nucleic acid or nucleic acid-protein complex into the patient.

In another preferred embodiment, a vector having nucleic acid sequences encoding a kinase polypeptide is provided in which the nucleic acid sequence is expressed only in specific tissue. Methods of achieving tissue-specific gene expression are set forth in International Publication No. WO 93/09236, filed Nov. 3, 1992 and published May 13, 1993.

In all of the preceding vectors set forth above, a further aspect of the invention is that the nucleic acid sequence contained in the vector may include additions, deletions or modifications to some or all of the sequence of the nucleic acid, as defined above.

Expression, including over-expression, of a kinase polypeptide of the invention can be inhibited by administration of an antisense molecule that binds to and inhibits expression of the mRNA encoding the polypeptide. Alternatively, expression can be inhibited in an analogous manner using a ribozyme that cleaves the mRNA. Alternatively, RNAi technology can be used. General methods of using antisense, ribozyme technology and RNAi technology, to control gene expression, or of gene therapy methods for expression of an exogenous gene in this manner are well known in the art. Each of these methods utilizes a system, such as a vector, encoding either an antisense or ribozyme transcript of a phosphatase polypeptide of the invention.

The term “ribozyme” refers to an RNA structure of one or more RNAs having catalytic properties. Ribozymes generally exhibit endonuclease, ligase or polymerase activity. Ribozymes are structural RNA molecules which mediate a number of RNA self-cleavage reactions. Various types of trans-acting ribozymes, including “hammerhead” and “hairpin” types, which have different secondary structures, have been identified. A variety of ribozymes have been characterized. See, for example, U.S. Pat. Nos. 5,246,921, 5,225,347, 5,225,337 and 5,149,796. Mixed ribozymes comprising deoxyribo and ribooligonucleotides with catalytic activity have been described. Perreault, et al., Nature, 344:565-567 (1990).

The term “RNAi” stands for RNA interference. This term is understood in the art to encompass technology using RNA molecules that can silence genes. See, for example, McManus, et al. Nature Reviews Genetics 3:737 (2002). In this application, the term “RNAi” encompasses molecules such as short interfering RNA (siRNA), microRNAs (mRNA), small temporal RNA (stRNA). Generally speaking, RNA interference results from the interaction of double-stranded RNA with genes.

As used herein, “antisense” refers of nucleic acid molecules or their derivatives which specifically hybridize, e.g., bind, under cellular conditions, with the genomic DNA and/or cellular mRNA encoding a phosphatase polypeptide of the invention, so as to inhibit expression of that protein, for example, by inhibiting transcription and/or translation. The binding may be by conventional base pair complementarity, or, for example, in the case of binding to DNA duplexes, through specific interactions in the major groove of the double helix. In one aspect, the antisense construct is an nucleic acid which is generated ex vivo and that, when introduced into the cell, can inhibit gene expression by, without limitation, hybridizing with the mRNA and/or genomic sequences of a kinase polynucleotide of the invention.

Antisense approaches can involve the design of oligonucleotides (either DNA or RNA) that are complementary to phosphatase polypeptide mRNA and are based on the kinase polynucleotides of the invention, including SEQ ID NOS:88-174. The antisense oligonucleotides will bind to the phosphatase polypeptide mRNA transcripts and prevent translation.

Although absolute complementarity is preferred, it is not required. A sequence “complementary” to a portion of an RNA, as referred to herein, means a sequence having sufficient complementarity to be able to hybridize with the RNA, forming a stable duplex; in the case of double-stranded antisense nucleic acids, a single strand of the duplex DNA may thus be tested, or triplex formation may be assayed. The ability to hybridize will depend on both the degree of complementarity and the length of the antisense nucleic acid. Generally, the longer the hybridizing nucleic acid, the more base mismatches with an RNA it may contain and still form a stable duplex (or triplex, as the case may be). One skilled in the art can ascertain a tolerable degree of mismatch by use of standard procedures to determine the melting point of the hybridized complex.

In general, oligonucleotides that are complementary to the 5′ end of the message, e.g., the 5′ untranslated sequence up to and including the AUG initiation codon, should work most efficiently at inhibiting translation. However, sequences complementary to the 3′ untranslated sequences of mRNAs have been shown to be effective at inhibiting translation of mRNAs as well. (Wagner, R. (1994) Nature 372:333). Antisense oligonucleotides complementary to mRNA coding regions are less efficient inhibitors of translation but could be used in accordance with the invention. Whether designed to hybridize to the 5′, 3′ or coding region of the phosphatase polypeptide mRNA, antisense nucleic acids should be at least six nucleotides in length, and are preferably less than about 100 and more preferably less than about 50 or 30 nucleotides in length. Typically they should be between 10 and 25 nucleotides in length. Such principles will inform the practitioner in selecting the appropriate oligonucleotides In preferred embodiments, the antisense sequence is selected from an oligonucleotide sequence that comprises, consists of, or consists essentially of about 10-30, and more preferably 15-25, contiguous nucleotide bases of a nucleic acid sequence selected from the group consisting of SEQ ID NOS:88-174 or domains thereof.

In another preferred embodiment, the invention includes an isolated, enriched or purified nucleic acid molecule comprising, consisting of or consisting essentially of about 10-30, and more preferably 15-25 contiguous nucleotide bases of a nucleic acid sequence that encodes a polypeptide that is selected from the group consisting of SEQ ID NOS:1-87.

Using the sequences of the present invention, antisense oligonucleotides can be designed. Such antisense oligonucleotides would be administered to cells expressing the target phosphatase and the levels of the target RNA or protein with that of an internal control RNA or protein would be compared. Results obtained using the antisense oligonucleotide would also be compared with those obtained using a suitable control oligonucleotide. A preferred control oligonucleotide is an oligonucleotide of approximately the same length as the test oligonucleotide. Those antisense oligonucleotides resulting in a reduction in levels of target RNA or protein would be selected.

The oligonucleotides can be DNA or RNA or chimeric mixtures or derivatives or modified versions thereof, single-stranded or double-stranded. The oligonucleotide can be modified at the base moiety, sugar moiety, or phosphate backbone, for example, to improve stability of the molecule, hybridization, etc. The oligonucleotide may include other appended groups such as peptides (e.g., for targeting host cell receptors in vivo), or agents facilitating transport across the cell membrane (see, e.g., Letsinger et al. (1989) Proc. Natl. Acad. Sci. U.S.A. 86:6553-6556; Lemaitre et al. (1987) Proc. Natl. Acad. Sci. USA 84:648-652; PCT Publication No. WO 88/09810, published Dec. 15, 1988) or the blood-brain barrier (see, e.g., PCT Publication No. WO 89/10134, published Apr. 25, 1988), hybridization-triggered cleavage agents. (See, e.g., Krol et al. (1988) BioTechniques 6:958-976) or intercalating agents. (See, e.g, Zon (1988) Pharm. Res. 5:539-549). To this end, the oligonucleotide may be conjugated to another molecule, e.g., a peptide, hybridization triggered cross-linking agent, transport agent, hybridization-triggered cleavage agent, etc.

The antisense oligonucleotide may comprise at least one modified base moiety which is selected from moieties such as 5-fluorouracil, 5-bromouracil, 5-chlorouracil, 5-iodouracil, hypoxanthine, xanthine, 4-acetylcytosine, and 5-(carboxyhydroxyethyl) uracil. The antisense oligonucleotide may also comprise at least one modified sugar moiety selected from the group including but not limited to arabinose, 2-fluoroarabinose, xylulose, and hexose.

In yet another embodiment, the antisense oligonucleotide comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof. (see also U.S. Pat. Nos. 5,176,996; 5,264,564; and 5,256,775)

In yet a further embodiment, the antisense oligonucleotide is an α-anomeric oligonucleotide. An α-anomeric oligonucleotide forms specific double-stranded hybrids with complementary RNA in which, contrary to the usual β-units, the strands run parallel to each other (Gautier et al. (1987) Nucl. Acids Res. 15:6625-6641). The oligonucleotide is a 2′-O-methylribonucleotide (Inoue et al. (1987) Nucl. Acids Res. 15:6131-6148), or a chimeric RNA-DNA analogue (Inoue et al. (1987) FEBS Lett. 215:327-330).

Also suitable are peptidyl nucleic acids, which are polypeptides such as polyserine, polythreonine, etc. including copolymers containing various amino acids, which are substituted at side-chain positions with nucleic acids (T,A,G,C,U). Chains of such polymers are able to hybridize through complementary bases in the same manner as natural DNA/RNA. Alternatively, an antisense construct of the present invention can be delivered, for example, as an expression plasmid or vector that, when transcribed in the cell, produces RNA complementary to at least a unique portion of the cellular mRNA which encodes a kinase polypeptide of the invention.

While antisense nucleotides complementary to the kinase polypeptide coding region sequence can be used, those complementary to the transcribed untranslated region are most preferred.

In another preferred embodiment, a method of gene replacement is set forth. “Gene replacement” as used herein means supplying a nucleic acid sequence which is capable of being expressed in vivo in an animal and thereby providing or augmenting the function of an endogenous gene which is missing or defective in the animal.

Pharmaceutical Formulations and Routes of Administration

The compounds described herein, including kinase polypeptides of the invention, antisense molecules, ribozymes, and any other compound that modulates the activity of a kinase polypeptide of the invention can be administered to a human patient per se, or in pharmaceutical compositions where it is mixed with other active ingredients, as in combination therapy, or suitable carriers or excipient(s). Techniques for formulation and administration of the compounds of the instant application may be found in “Remington's Pharmaceutical Sciences,” Mack Publishing Co., Easton, Pa., latest edition.

Routes of Administration:

Suitable routes of administration may, for example, include oral, rectal, transmucosal, or intestinal administration; parenteral delivery, including intramuscular, subcutaneous, intravenous, intramedullary injections, as well as intrathecal, direct intraventricular, intraperitoneal, intranasal, or intraocular injections.

Alternately, one may administer the compound in a local rather than systemic manner, for example, via injection of the compound directly into a solid tumor, often in a depot or sustained release formulation.

Furthermore, one may administer the drug in a targeted drug delivery system, for example, in a liposome coated with tumor-specific antibody. The liposomes will be targeted to and taken up selectively by the tumor.

Composition/Formulation:

The pharmaceutical compositions of the present invention may be manufactured in a manner that is itself known, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in conventional manner using one or more physiologically acceptable carriers comprising excipients and auxiliaries which facilitate processing of the active compounds into preparations which can be used pharmaceutically. Proper formulation is dependent upon the route of administration chosen.

For injection, the agents of the invention may be formulated in aqueous solutions, preferably in physiologically compatible buffers such as Hanks's solution, Ringer's solution, or physiological saline buffer. For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated. Suitable carriers include excipients such as, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

For buccal administration, the compositions may take the form of tablets or lozenges formulated in conventional manner.

For administration by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

Additionally, the compounds may be delivered using a sustained-release system, such as semipermeable matrices of solid hydrophobic polymers containing the therapeutic agent. Various sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Many of the tyrosine or serine/threonine kinase modulating compounds of the invention may be provided as salts with pharmaceutically compatible counterions. Pharmaceutically compatible salts may be formed with many acids, including but not limited to hydrochloric, sulfuric, acetic, lactic, tartaric, malic, succinic, etc. Salts tend to be more soluble in aqueous or other protonic solvents that are the corresponding free base forms.

Suitable Dosage Regimens:

Pharmaceutical compositions suitable for use in the present invention include compositions where the active ingredients are contained in an amount effective to achieve its intended purpose. More specifically, a therapeutically effective amount means an amount of compound effective to prevent, alleviate or ameliorate symptoms of disease or prolong the survival of the subject being treated. Determination of a therapeutically effective amount is well within the capability of those skilled in the art, especially in light of the detailed disclosure provided herein.

The proper dosage depends on various factors such as the type of disease being treated, the particular composition being used and the size and physiological condition of the patient. Therapeutically effective doses for the compounds described herein can be estimated initially from cell culture and animal models. For example, a dose can be formulated in animal models to achieve a circulating concentration range that initially takes into account the IC₅₀ as determined in cell culture assays. The animal model data can be used to more accurately determine useful doses in humans.

For any compound used in the methods of the invention, the therapeutically effective dose can be estimated initially from cell culture assays. For example, a dose can be formulated in animal models to achieve a circulating concentration range that includes the IC₅₀ as determined in cell culture (i.e., the concentration of the test compound which achieves a half-maximal inhibition of the tyrosine or serine/threonine kinase activity). Such information can be used to more accurately determine useful doses in humans.

Toxicity and therapeutic efficacy of the compounds described herein can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio between LD₅₀ and ED₅₀. Compounds which exhibit high therapeutic indices are preferred. The data obtained from these cell culture assays and animal studies can be used in formulating a range of dosage for use in human. The dosage of such compounds lies preferably within a range of circulating concentrations that include the ED₅₀ with little or no toxicity. The dosage may vary within this range depending upon the dosage form employed and the route of administration utilized. The exact formulation, route of administration and dosage can be chosen by the individual physician in view of the patient's condition. (See e.g., Fingl et al., 1975, in “The Pharmacological Basis of Therapeutics”, Ch. 1 p.1).

In another example, toxicity studies can be carried out by measuring the blood cell composition. For example, toxicity studies can be carried out in a suitable animal model as follows: 1) the compound is administered to mice (an untreated control mouse should also be used); 2) blood samples are periodically obtained via the tail vein from one mouse in each treatment group; and 3) the samples are analyzed for red and white blood cell counts, blood cell composition and the percent of lymphocytes versus polymorphonuclear cells. A comparison of results for each dosing regime with the controls indicates if toxicity is present.

At the termination of each toxicity study, further studies can be carried out by sacrificing the animals (preferably, in accordance with the American Veterinary Medical Association guidelines Report of the American Veterinary Medical Assoc. Panel on Euthanasia:229-249, 1993). Representative animals from each treatment group can then be examined by gross necropsy for immediate evidence of metastasis, unusual illness or toxicity. Gross abnormalities in tissue are noted and tissues are examined histologically. Compounds causing a reduction in body weight or blood components are less preferred, as are compounds having an adverse effect on major organs. In general, the greater the adverse effect the less preferred the compound.

Plasma levels should reflect the potency of the drug. Generally, the more potent the compound the lower the plasma levels necessary to achieve efficacy.

Plasma half-life and biodistribution of the drug and metabolites in the plasma, tumors and major organs can also be determined to facilitate the selection of drugs most appropriate to inhibit a disorder. Such measurements can be carried out. For example, HPLC analysis can be performed on the plasma of animals treated with the drug and the location of radiolabeled compounds can be determined using detection methods such as X-ray, CAT scan and MRI. Compounds that show potent inhibitory activity in the screening assays, but have poor pharmacokinetic characteristics, can be optimized by altering the chemical structure and retesting. In this regard, compounds displaying good pharmacokinetic characteristics can be used as a model.

Dosage amount and interval may be adjusted individually to provide plasma levels of the active moiety which are sufficient to maintain the kinase modulating effects, or minimal effective concentration (MEC). The MEC will vary for each compound but can be estimated from in vitro data; e.g., the concentration necessary to achieve 50-90% inhibition of the kinase using the assays described herein. Dosages necessary to achieve the MEC will depend on individual characteristics and route of administration. However, HPLC assays or bioassays can be used to determine plasma concentrations.

Dosage intervals can also be determined using MEC value. Compounds should be administered using a regimen which maintains plasma levels above the MEC for 10-90% of the time, preferably between 30-90% and most preferably between 50-90%.

In cases of local administration or selective uptake, the effective local concentration of the drug may not be related to plasma concentration.

The amount of composition administered will, of course, be dependent on the subject being treated, on the subject's weight, the severity of the affliction, the manner of administration and the judgment of the prescribing physician.

Packaging:

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the polynucleotide for human or veterinary administration. Such notice, for example, may be the labeling approved by the U.S. Food and Drug Administration for prescription drugs, or the approved product insert. Compositions comprising a compound of the invention formulated in a compatible pharmaceutical carrier may also be prepared, placed in an appropriate container, and labeled for treatment of an indicated condition. Suitable conditions indicated on the label may include treatment of a tumor, inhibition of angiogenesis, treatment of fibrosis, diabetes, and the like.

### EXAMPLES

The examples below are not limiting and are merely representative of various aspects and features of the present invention. The examples below demonstrate the isolation and characterization of the nucleic acid molecules according to the invention, as well as the polypeptides they encode.

Methods and Examples for Determining Secondary Structure and CSSP

Secondary structure can be derived from known three-dimensional structures using DSSP program/database protein sequences. Kabsch et al. Dictionary of protein secondary structure: Pattern recognition of hydrogen-bonded and geometrical features, 22:2577 (1983).

Secondary structure predictions can be obtained using the program PSIPRED (Jones, 1999). The program converts evolutionary predictions derived from multiple sequence alignments into secondary structures using neural networks. The profiles, also called position-specific substitution matrices have been derived for each protein sequence analyzed using ten iterations of a PSI-BLAST search on a non-redundant database of public protein sequences (available from National Center for Biotechnology Information (NCBI)) with e-value cutoff=1e-5 (Altschul et al, 1997). To improve the quality of the secondary structure profiles we masked all low-complexity, transmembrane and coiled-coil regions in the protein sequence NCBI database using a program PFILT (a part of PSIPRED package (Jones, 1999)).

Secondary structure conformation can be predicted or derived from known three-dimensional protein structure for each amino acid residue in the polypeptide chain. A sequence of residues with same secondary structure conformation (without breaks) can be defined as a single secondary structure element. We distinguish three types of secondary structure elements: helix (denoted by h), beta-strand (denoted by e), and loop (denoted by underscore) connecting any of the two former types. We describe the secondary structure pattern as a sequence of secondary structure elements. For each helix and strand predicted next to each and not separated by loop, we put a loop of null size between the helix and/or strand, which reduces variety of secondary structure patterns.

### Example 1

This example illustrates deriving CSSP for two kinase domains from Protein Data Bank (PDB; Bernstein et al., 1977; Berman et al, 2000), namely: 1vr2a (Mctigue et al., 1999) and lagwa (Mohammadi et al., 1997). The amino acid sequences of these kinases are shown below.

Sequence of lvr2a
LPYDASKWEFPRDRLKLGKPLGRGAFGQVIEADAFGIDKTATCRTVAVKM
LKEGATHSEHRALMSELKILIHTGHHLNVVNLLCACTKPGGPLMVIVEFC
KFGNLSTYLRSKRNEFVPYKVAPEDLYKDFLTLEHLICYSFQVAKGMEFL
ASRKCIHRDLAARNILLSEKNVVKICDFGLARDIYKDPDXVRKGDARLPL
KWMAPETIFDRVYTTQSDVWSFGVLLWEIFSLGASPYPGVKIDEEFCRRL
KEGTRMRAPDYTTPEMYQTMLDCWHGEPSQRPTFSELVEHLGNLLQANA
Sequence of lagwa
ELPEDPRWELPRDRLVLGKPLGEGAFGQVVLAEAIGLDKDKPNRVTKVAV
KMLKSDATEKDLSDLISEMEMMKMIGKHKNIINLLGACTQDGPLYVIVEY
ASKGNLREYLQARRPPGLEYSYNPSHNPEEQLSSKDLVSCAYQVARGMEY
LASKKCIHRDLAARNVLVTEDNVMKIADFGLARDIHHIDYYKKTTNGRLP
VKWMAPEALFDRIYTHQSDVWSFGVLLWEIFTLGGSPYPGVPVEELFKLL
KEGHRMDKPSNCTNELYMMMRDCWHAVPSQRPTFKQLVEDLDRIVALTS

The secondary structures for these kinases are predicted using PSIPRED:

lvr2a
cccccccccccccceeeeeeehhccceeeeeeeeecccccccceeeeeee
cccccchhhhhhhhhhhhhhhhccccccceeeeeeeeccccceeeeeccc
cccchhhhhhhccccccccccccccccccccchhhhhhhhhhhhhhhhhh
hcccccccccccccceecccceeeeeccccceecccccceeccccccccc
ccchhhhhhccccccceeeeecceehhhhhhccccccccccchhhhhhhh
hcccccccccccchhhhhhhhhhhhcccccccchhhhhhhhhhhhcccc
lagwa
cccccccccccccceeeeeeeecccceeeeeeeeeccccccccccceeee
eecccccchhhhhhhhhhhhhhhhccccccceeeeeeeecccceeeeecc
ccccchhhhhhhccccccccccccccccccccchhhhhhhhhhhhhhhhh
hhccccccccccccceeecccceeeeeccccchhhcccceeeeccccccc
cccchhhhhhccccccceeeeecceehhhhhhcccccccccchhhhhhhh
hcccccccccccchhhhhhhhhhhhcccccccchhhhhhhhhhhhhcccc
ccccccccchhhhhcccccccccccccc

These predicted secondary structures can be converted into the pattern form as follows:

| | |
|---|---|
| _e_h_e_e_h_e_e_h_h_e_e_e_e_h_e_e_h_h_h_h_{—} | lvr2a_{—} |
| _e_e_e_h_e_e_h_h_e_e_h_e_h_e_e_h_h_h_h_{—} | lagwa_{—} |

The consensus secondary structure pattern, present in two kinases is:

e_e_h_e_e_h_h_e_e

### Example 2

This example shows variety of CSSP in known protein kinases. To derive secondary structure patterns present in protein kinases we used 99 kinase domains of known structures as indicated by SCOP, a manually created database of known three-dimensional structures (Murzin et al., 1995). The secondary structures of these kinases (both observed using DSSP and predicted by PSIPRED) have been converted into 99 secondary structure patterns.

By visual inspection of those patterns we concluded that although the full patterns of each protein kinase are different from each other, most of them share a conserved part:

| | | |
|---|---|---|
| Pattern: | # | Type: |

most frequent:

| | | |
|---|---|---|
| ...e_e_h_e_e_h_h_e_e... | 66 | standard |
| ...e_e_e_h_h_e_e_h_h_e_e... | 15 | CAMP |
| ...e_e_e_h_e_e_e_h_h_e_e... | 9 | CDK2/MAP |
| ...e_e_e_h_e_e_h_h_h_e... | 4 | FGFR1, second |
| | | domain |

less frequent:

| | | |
|---|---|---|
| ...e_e_e_h_e_e_h_e_e_e... | 2 | ser/thr kinase, |
| | | 2nd domain |
| ...e_e_h_e_h_h_h_e... | 2 | actin/fragmin |
| | | kinase |
| ...e_e_h_e_e_e_h_h_h_e_e... | 1 | P13K |

The four most frequently occurring patterns describe 94 (95%) of the 99 kinase domains of known structure in the SCOP database (Murzin et al., 1995) and 408 (82%) of the 497 human protein kinases. These four CSSP were used further for kinase CRISSP.

Methods for Determining CAAR and CASAAR

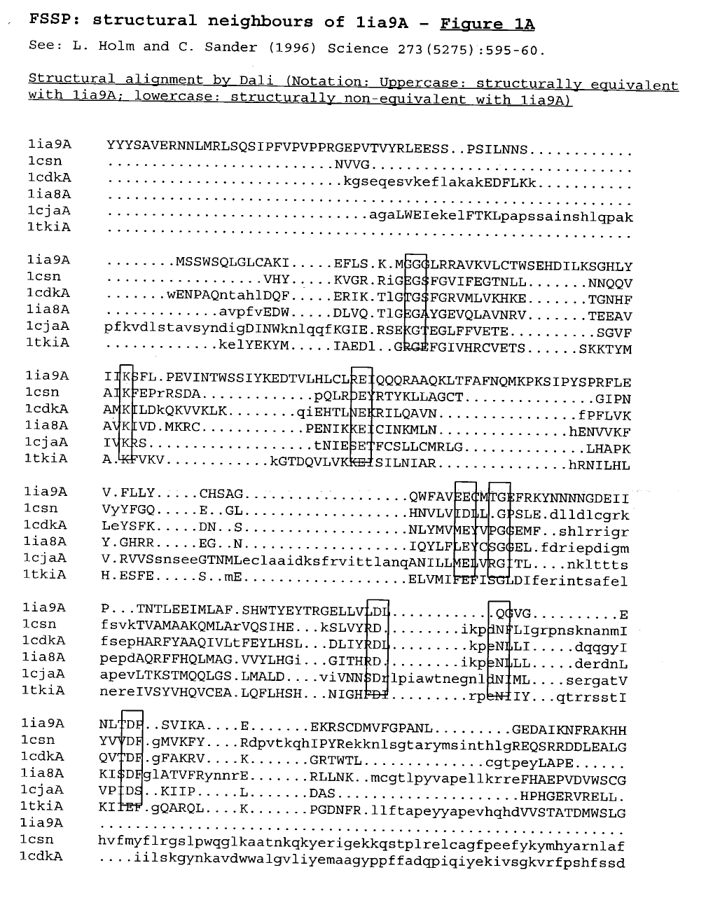

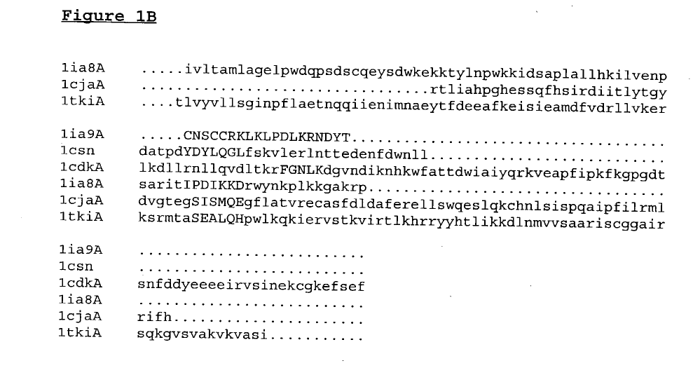

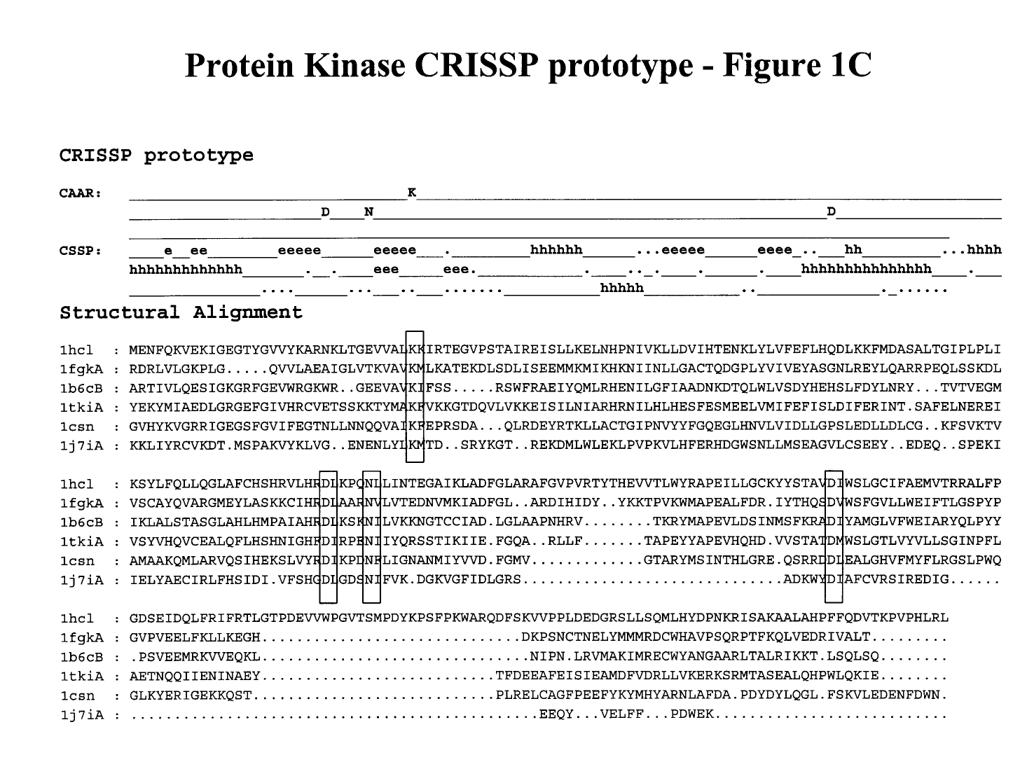

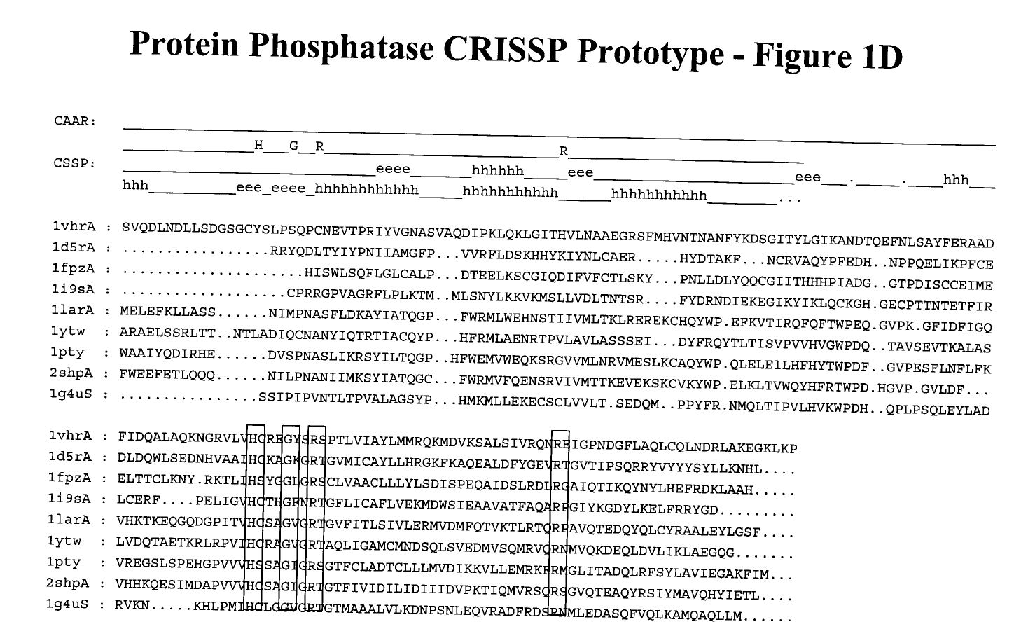

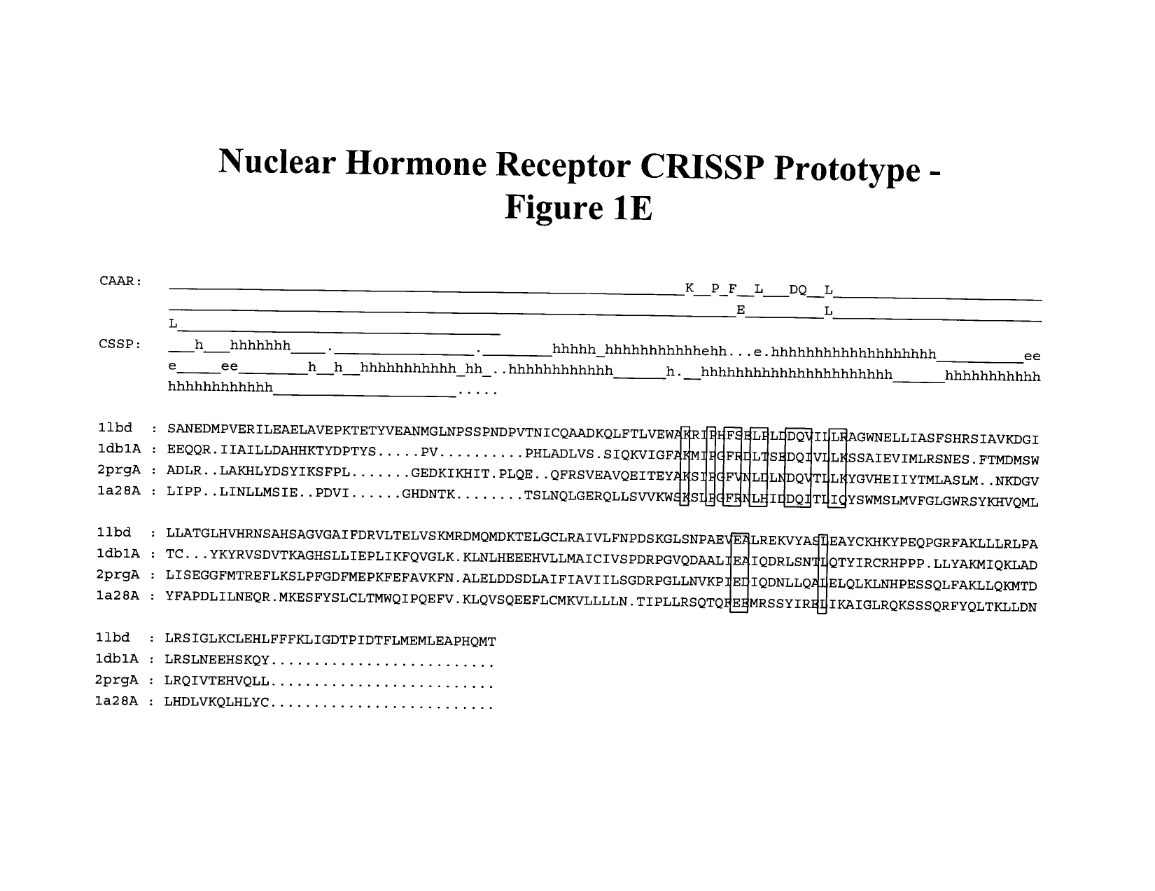

Secondary structure signatures reflect the conserved part of the kinase fold. Besides structural conservation, functionally and structurally important amino acid residues are also conserved (see CAAR and CASAAR above). To derive the conserved kinase residues we analyzed six kinases remotely homologous to each other and analyzed their structural alignments using FSSP database (Holm & Sanders, 1996) as shown on the Figure. On linear representation of three-dimensional superimposition of the six structures, eight residues demonstrate substantial conservation. Five of these residues, including catalytic Lys, appeared to be active site residues interacting with the phosphate group of the ATP molecule as indicated by analysis of known complexes of kinase structures with ligands (ATP substitutes) as well as multiple sequence alignments and biochemical data (e.g., Shi et al., 1998). See FIGS. 1A and 1B

Methods for Determining CRISSP

The residues conserved in multiple alignments can be projected onto secondary structure representation of the corresponding polypeptide sequences and unambiguously linked to the corresponding secondary structure elements.

The five conserved active site residues (K,E,D,N,D) are located in the conserved secondary structures identified above. However, due to variability of the secondary structure patterns and low quality of the secondary structure predictions for last two to four ‘e’ in ‘e_e_h_e_e_h_h_e_e’ (shown in italics) did not permit the superimposition of D,N,D onto that e_e region. Since the exact structural context for only two of the five amino acids is universal in all the pattern variations, we used the two first residues (K,E) to determine the CRISSP pattern. The remaining conserved residues can be used for further evaluation of the kinase predictions.

### Example 3a

This example illustrates superimposing CSSP and CASAAR for kinase domain lvr2a (Mctigue et al., 1999). The conserved amino acid residues (shown in bold) are located on a beta-strand and the next helix (both in bold) in the predicted secondary structure, its pattern, and the conserved part of the pattern.

Predicted secondary structure
cccccccccccccceeeeeeehhccceeeeeeeeecccccccceeeeeee
cccccchhhhhhhhhhhhhhhhccccccceeeeeeeeccccceeeeeccc
cccchhhhhhhccccccccccccccccccccchhhhhhhhhhhhhhhhhh
hcccccccccccccceecccceeeeeccccceecccccceeccccccccc
ccchhhhhhccccccceeeeecceehhhhhhccccccccccchhhhhhhh
hcccccccccccchhhhhhhhhhhhcccccccchhhhhhhhhhhhcccc
Secondary structure pattern (CSSP in italic):
_e_h_e_ [symbol] _ [symbol] _e_e_h_h_e_e_e_e_h_e_e_h_h_h_h_ lvr2a_
CRISS P:
e_e_h_e_e_h_h_e_e
_KE

### Example 3b

The example illustrates how to derive a CRISSP prototype from a FSSP database. A computer program can identify all proteins of known three-dimensional structure from a protein database (PDB) that are members of the same protein family (as defined in SCOP classification) and that relate to each other as remote homologues (for example, they share less than 25% of identical amino acid residues as derived from structural alignments). Structural alignments from FSSP can be processed for this group in order to derive CAAR of their amino acid sequences, CSSP of their secondary structures derived from the three-dimensional structures and superimpose them on the basis of the structural alignments.

FIG. 1C illustrates this approach to derive a CRISSP prototype for protein kinases. FIG. 1D illustrates this approach to derive a prototype CRISSP for protein phosphatases and FIG. 1E illustrates this approach to derive a prototype for a nuclear hormone receptors.

### Example 4

### Method and Example of Application of CRISSP to Protein Database

The kinase CRISSP patterns (see above) were tested on a dataset of 4486 structural domains, including 22 kinase domains. This dataset was formed as a subset of all known structural domains in SCOP database (Murzin et al., 1995) by excluding close homologues (sequence identity >95%)using ASTRAL (Brenner et al, 2000). Secondary structures for each of the 4486 domains were predicted using PSIPRED (Jones, 1999).

Out of 22 kinases in the dataset we found 16 kinases, with 2 false “kinase” positives. The false positives corresponds to the two least frequent patterns out of the used four kinase CRISSP. This corresponds to 73% specificity with 10% error rate. Extending the patterns allows us to achieve 0% error rate on the selected data set but also decreases the specificity to 55%.

### Example 5

### Method and Example of the Application of CRISSP Method to the Human Genome Database

Applied to the Celera human genome with predicted secondary structures for each open reading frame (ORF), kinase patterns were found in 445 ORFs. 350 them also can be detected by kinase Hidden Markov Model (HMM) from PFAM database (Sonnhammer et al., 1997). Thus, the Markov Model detects about 70% of the kinases identified using CRISSP, which is similar to the specificity rate using structural domains.

87 of the predictions we made using CRISSP cannot be detected by HMM (both local and global) and can be considered as novel predictions. Functionally annotated proteins (other than kinases) might be potentially considered as false positives. However, in some cases location of functionally annotated domains is not inconsistent with a predicted kinase domain; in others annotation is very broad (e.g., fragile X mental retardation protein 1 related) or does not interfere with kinase functions (e.g., channel protein might be channel kinase).

40 proteins were known as hypothetical in public databases. Most importantly, for eight proteins we found additional evidence in support of their kinase function. This evidence includes one or more of the following properties for each of the novel kinases:

GxGxxG or similar sequence motif in front of the active site residues;

The presence of the remaining 3-4 conserved residues originally not included in our CRISSP (note that above, we used two of the eight conserved residues);

Conservation of some or all active site residues in multiple alignments of the predicted kinases with their remote homologues;

The presence of other signaling domains such as SH2, SH3, SAM, etc;

Weak sequence similarity to known kinases (not considered as reliable by BLAST criteria);

Homology to a typical kinases not detectable by HMM;

Positive 3D threading results (kinase fold predicted among the 10 highest-scored folds; conservation of active site residues in the predicted alignment)

### Example 6

### Identification of a Putative Archeal Kinase

Using the CRISSP method described herein, we predicted protein kinase activity to a publicly known protein (gi|12741100 in NCBI database). This protein has homologous polypeptides encoded in several eukaryote and archea genomes but does not show homology to known protein kinases. These proteins form RI01 family, detectable by RIO1 PFAM HMM but not by PFAM kinase HMM. However, a putative archeal kinase was speculated to be the ancestor of eukaryotic protein kinases (Leonard et al., 1998). Our analysis of RI01 protein family shows that it has the same secondary structural and active site residue patterns (CRISSP) as eukaryotic protein kinases.

### Example 7

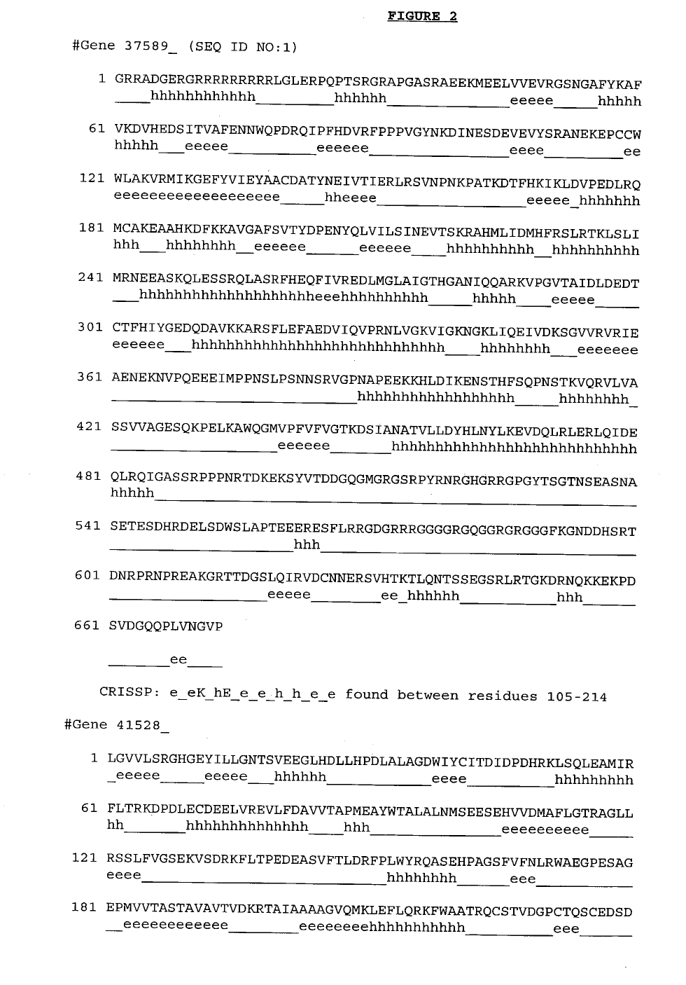

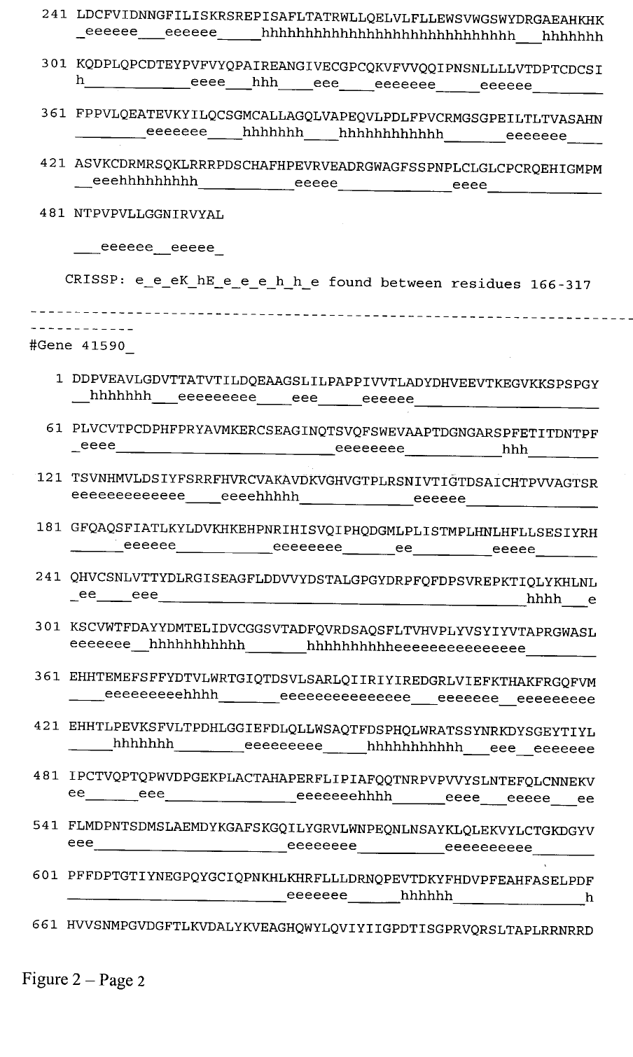

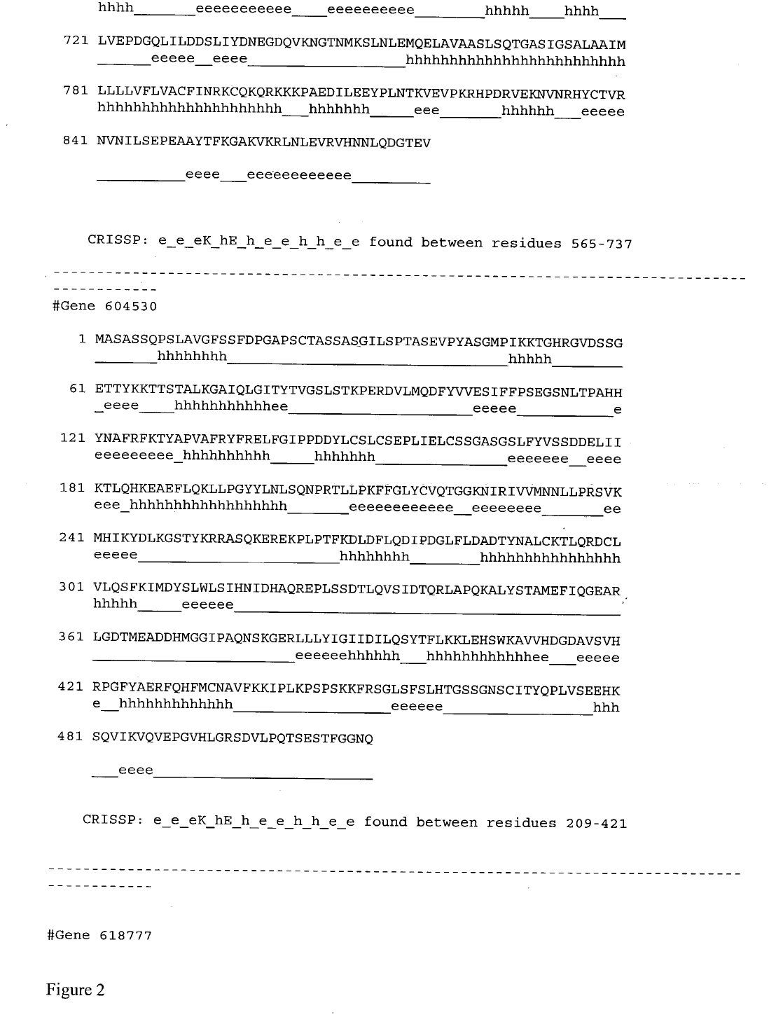

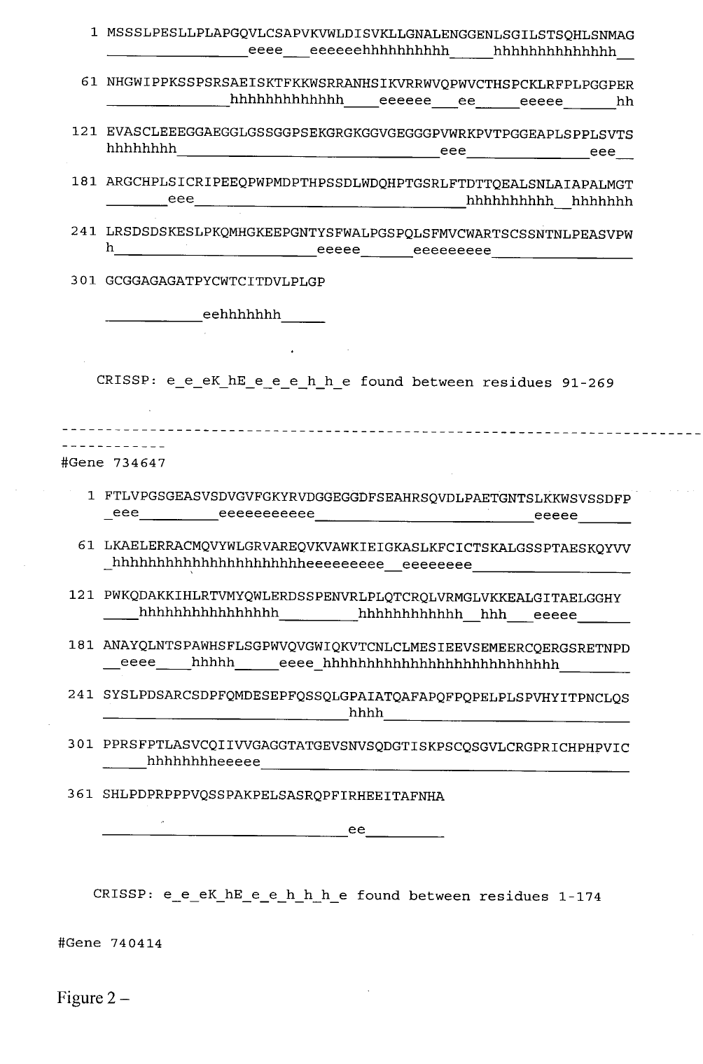

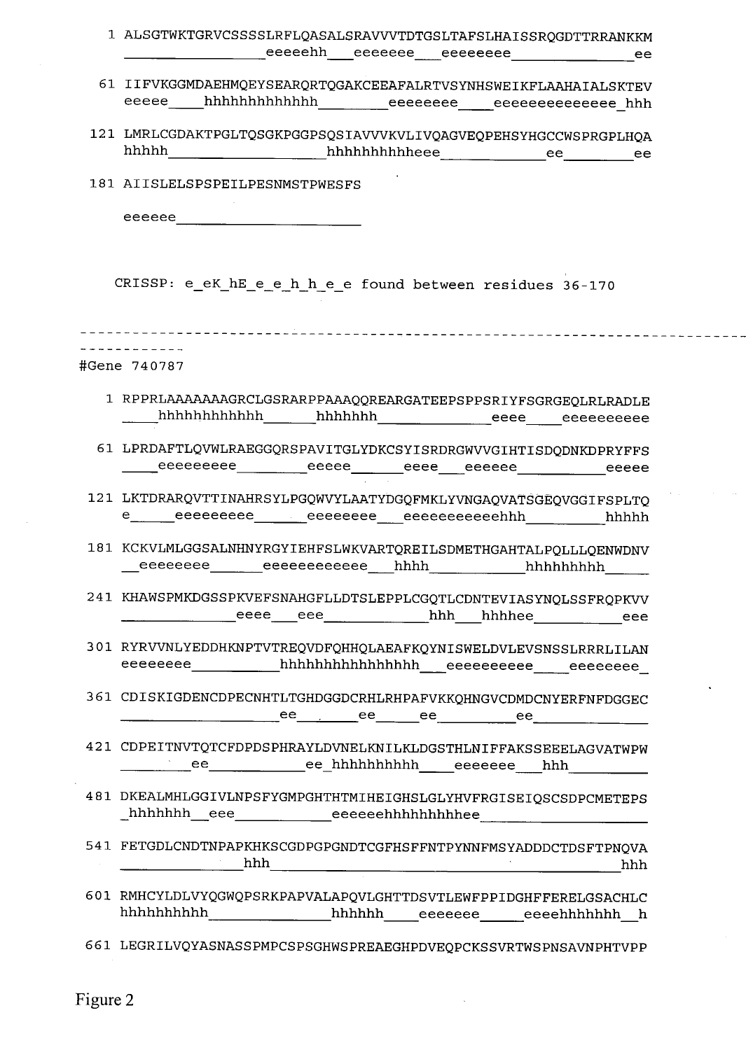

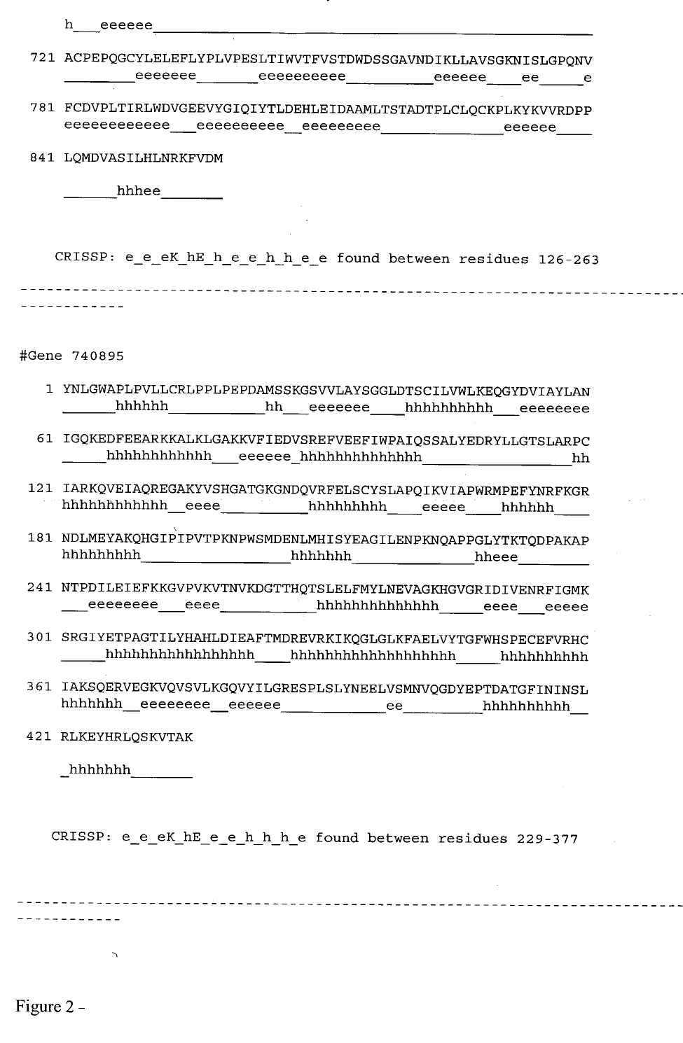

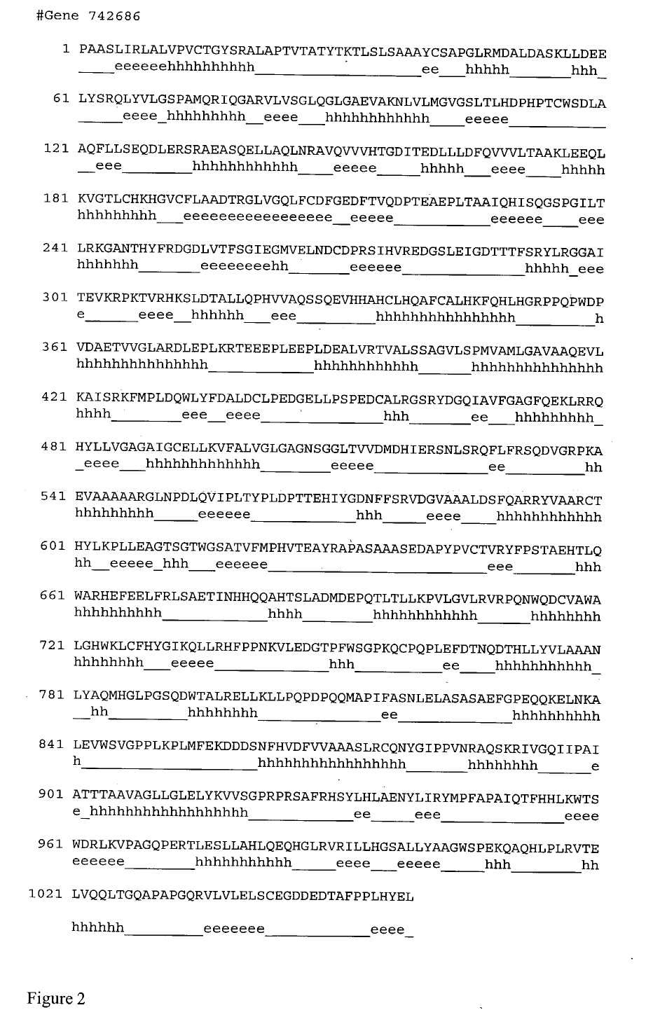

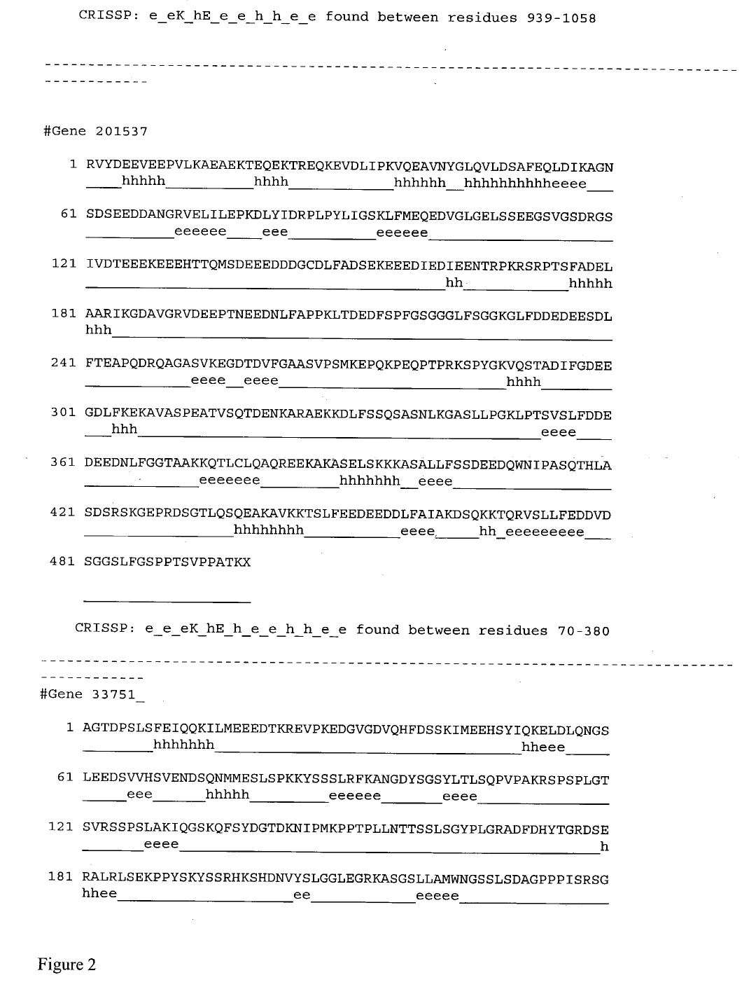

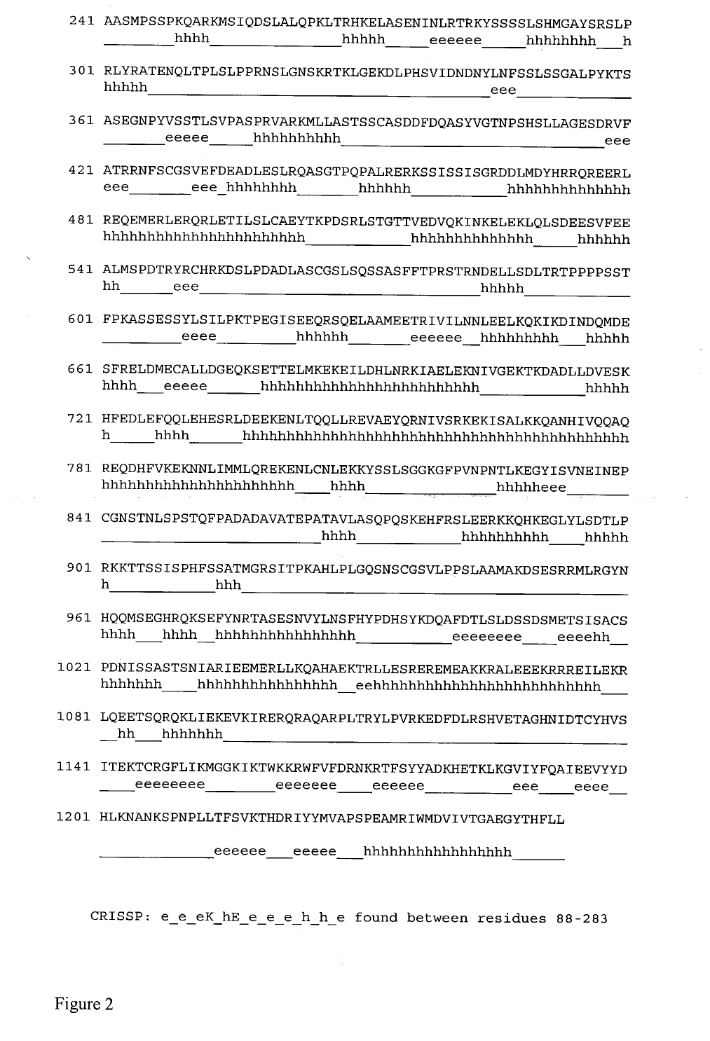

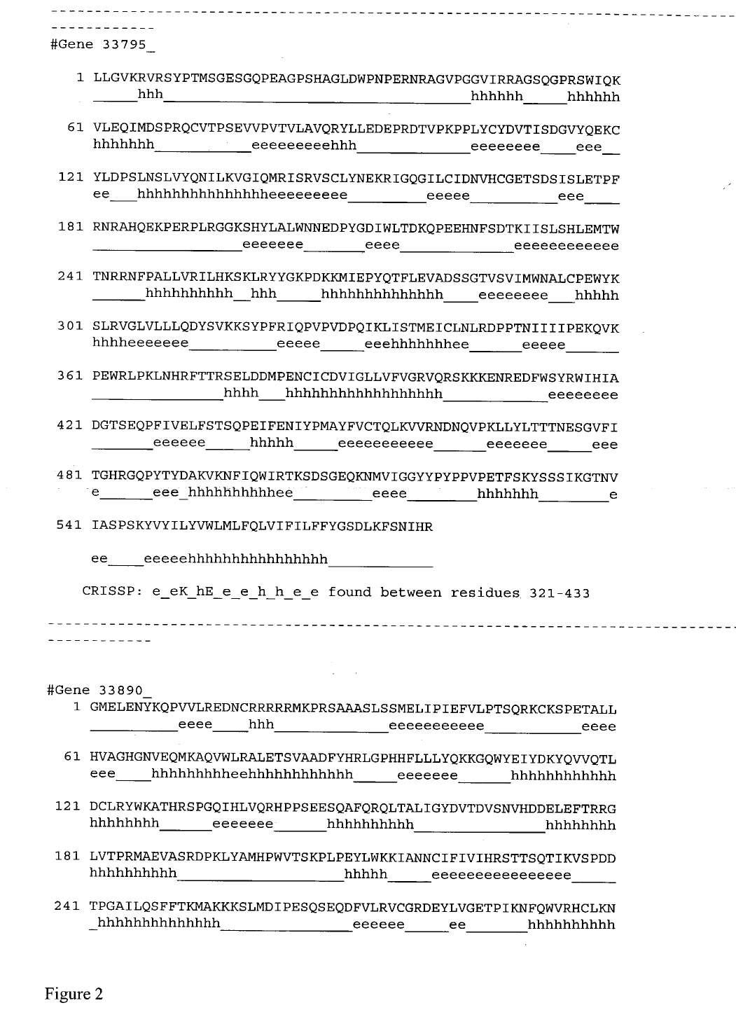

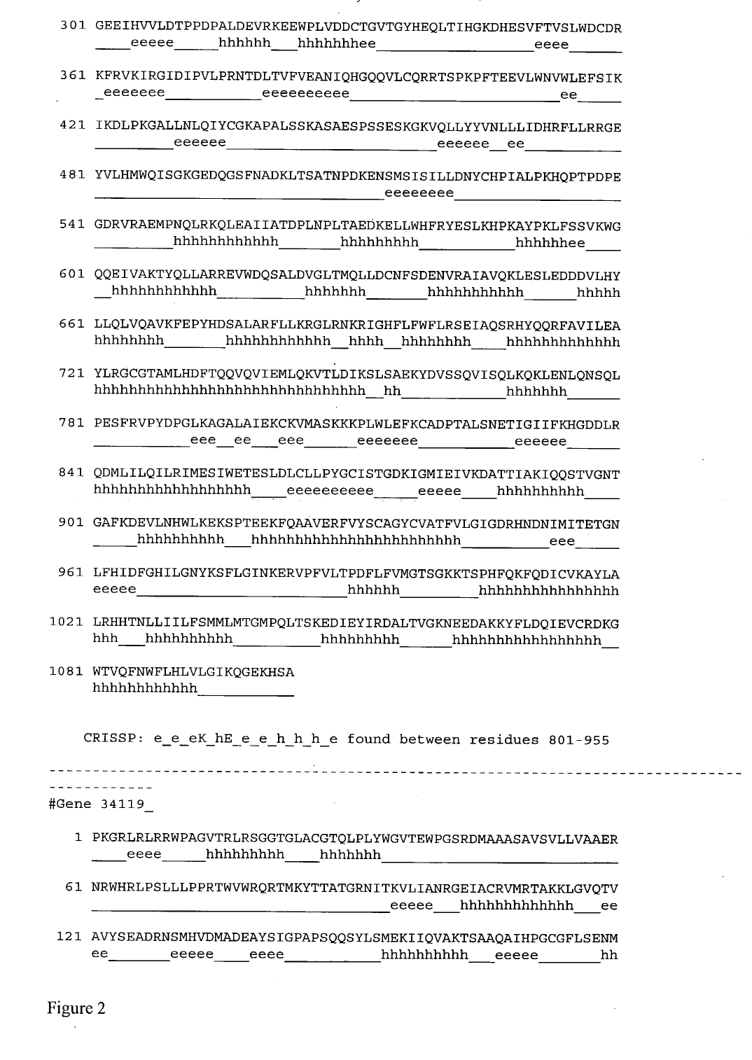

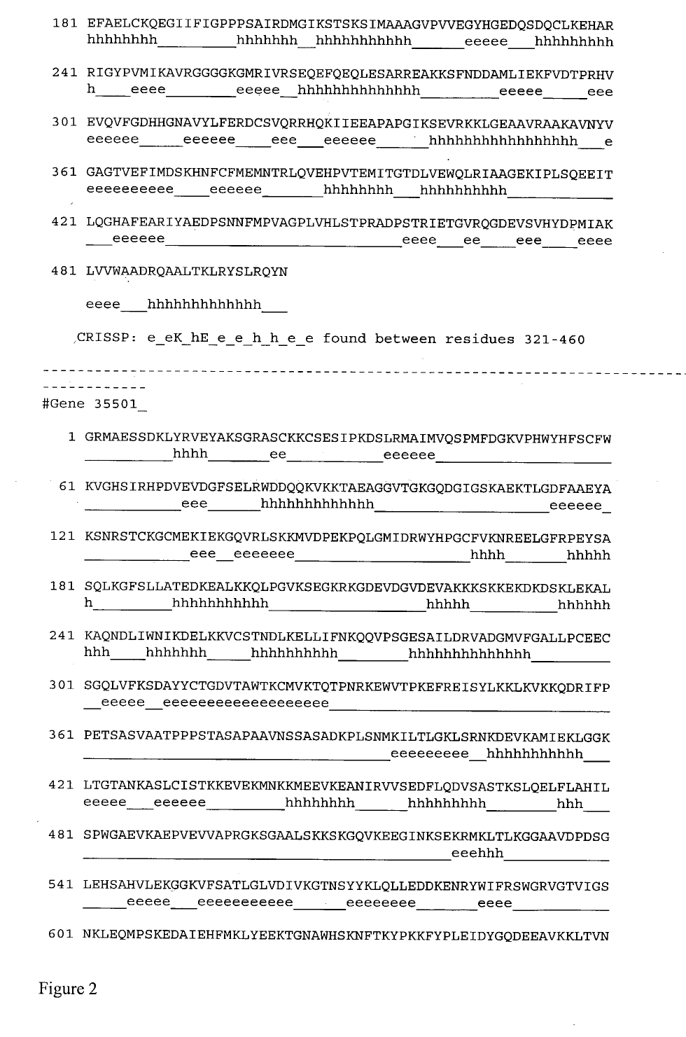

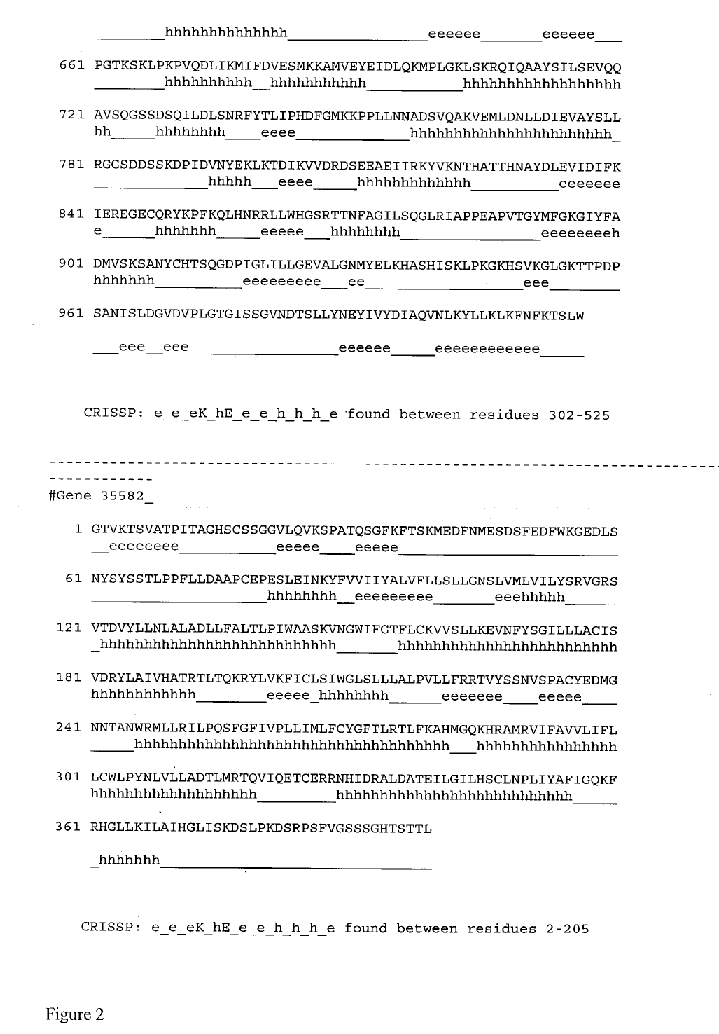

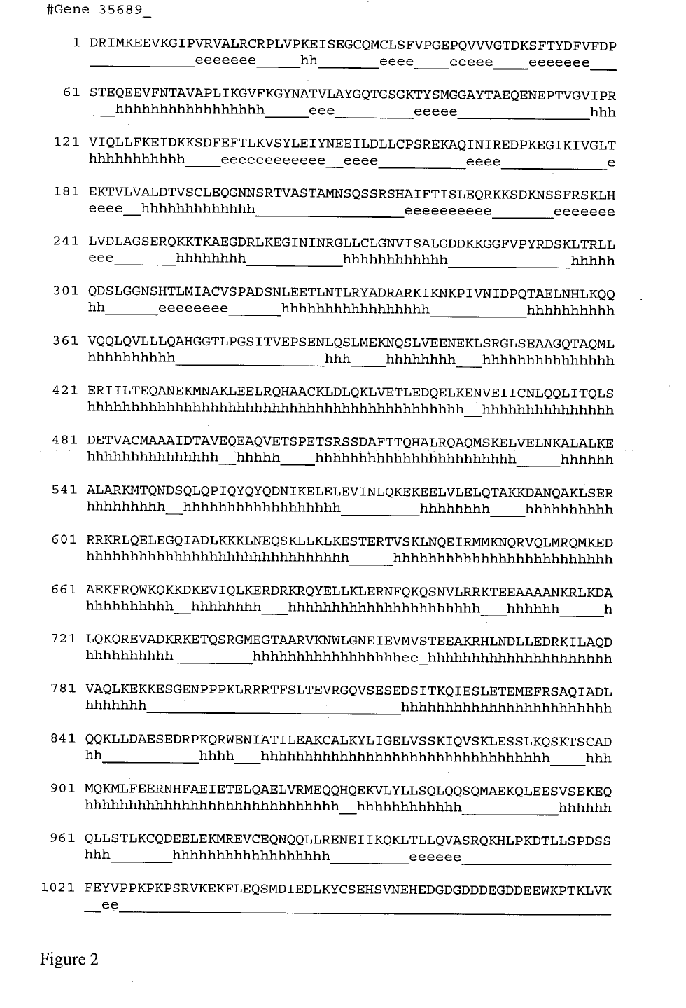

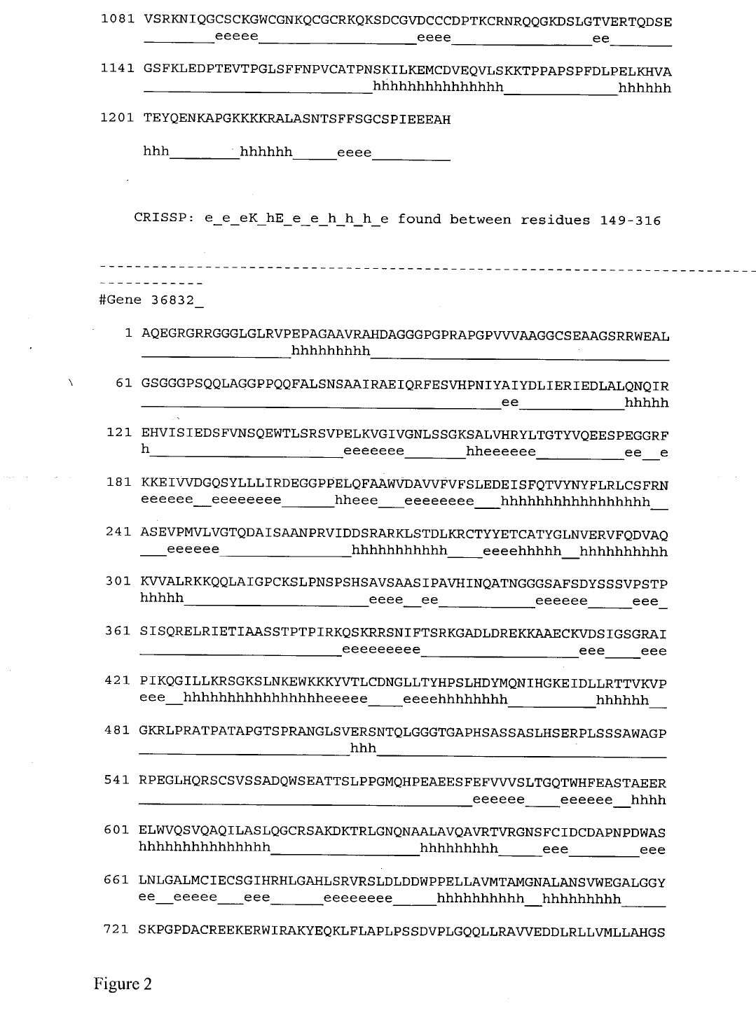

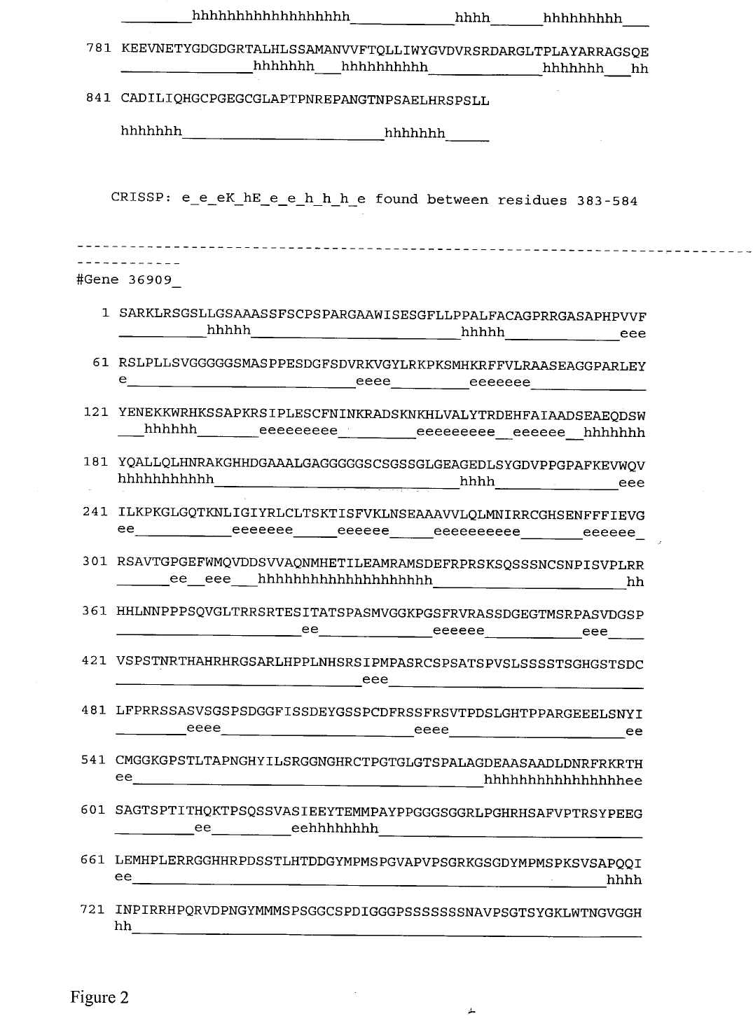

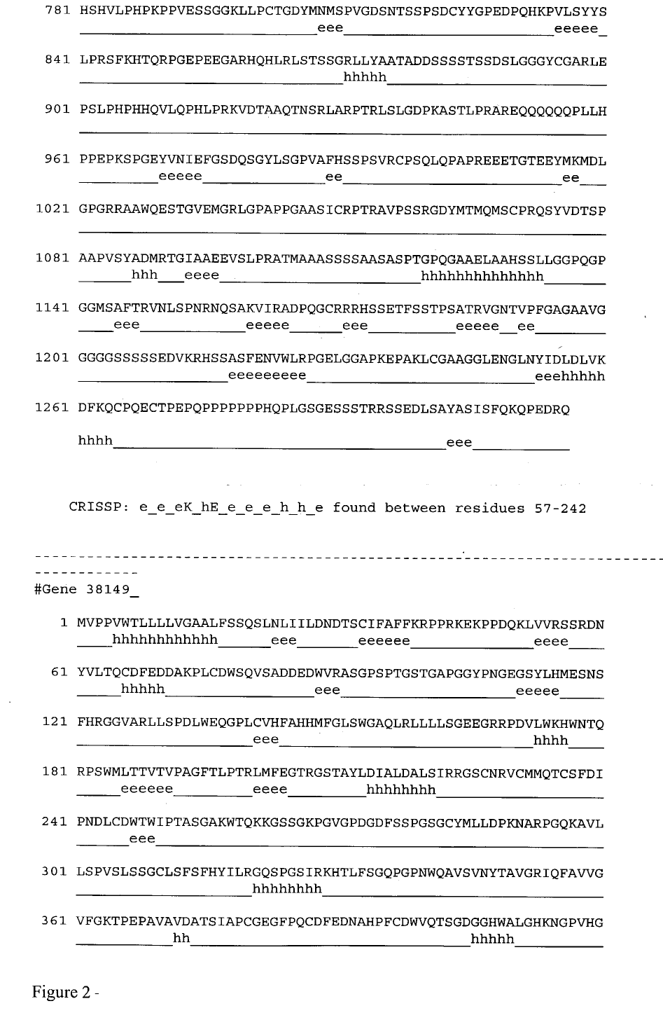

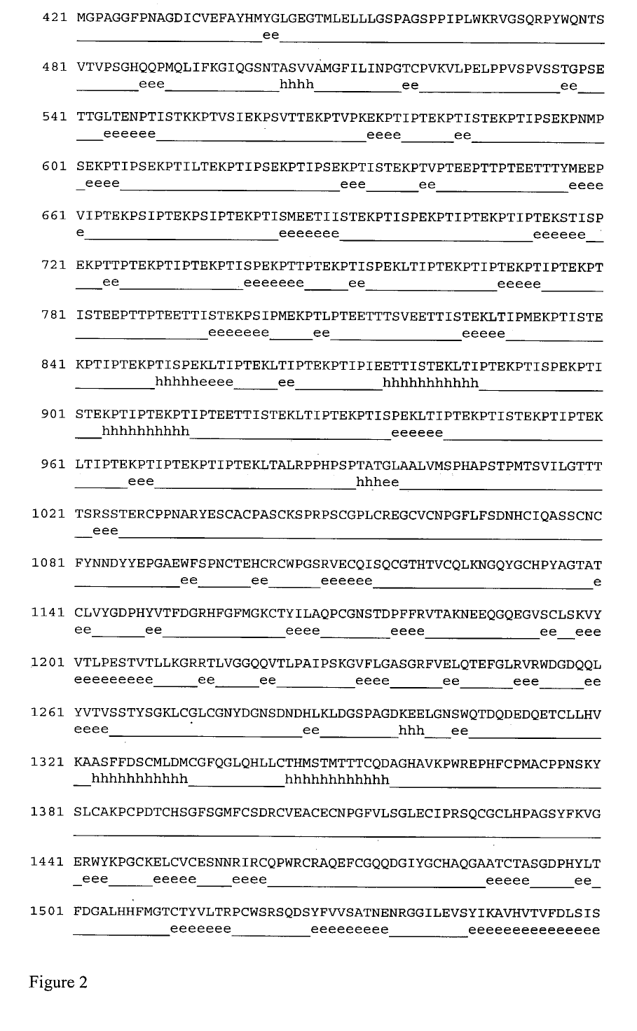

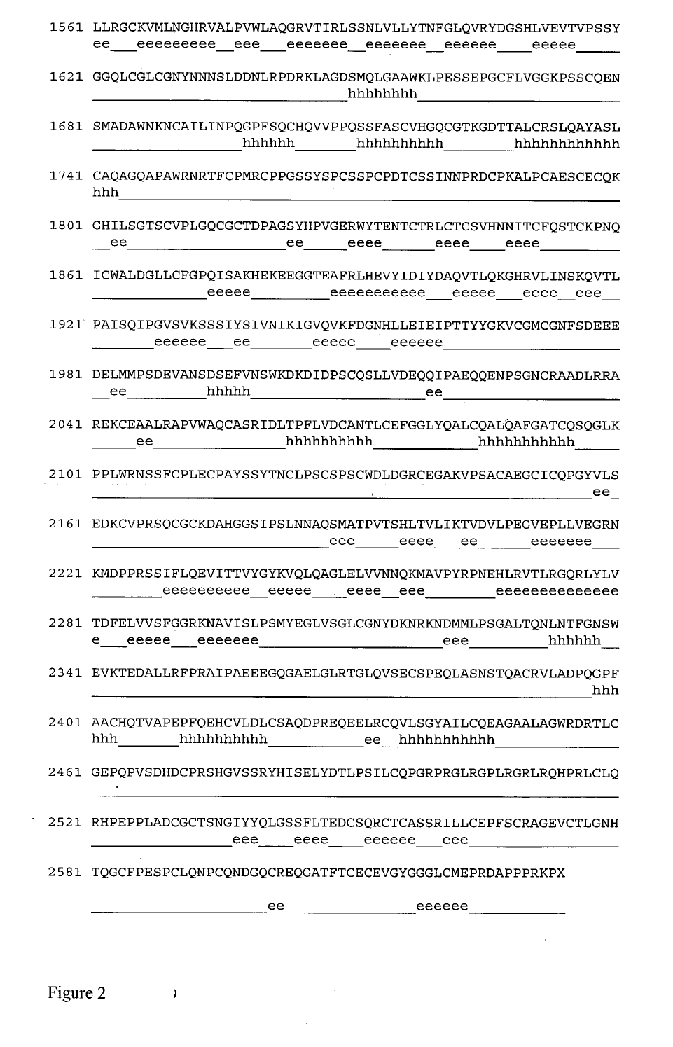

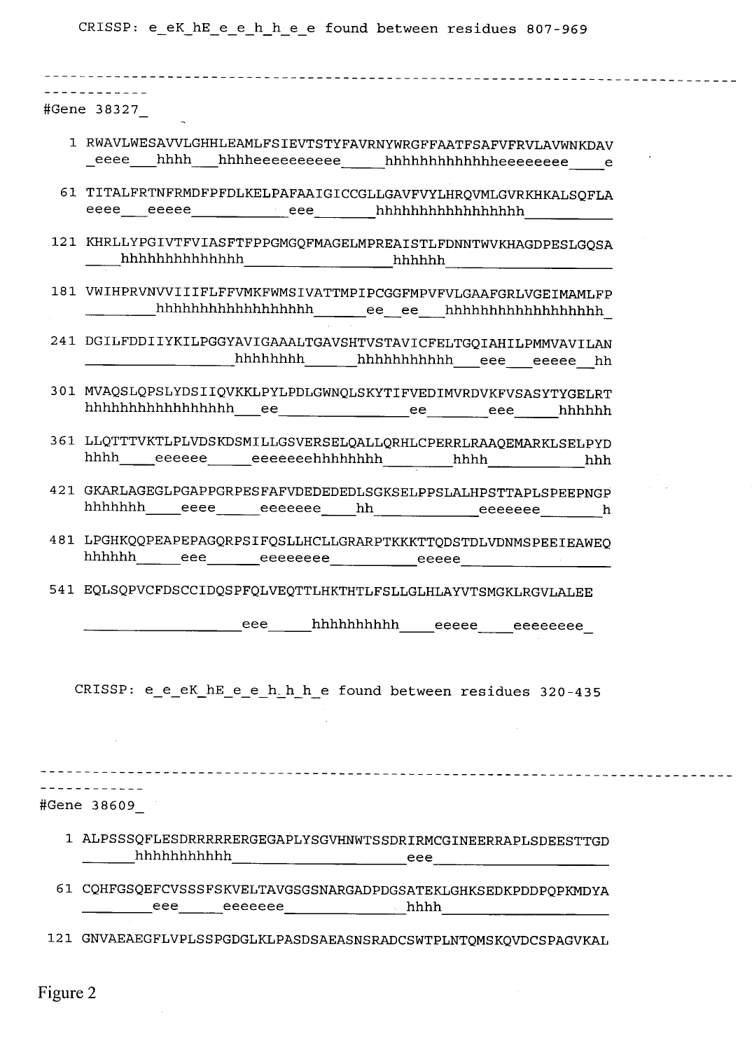

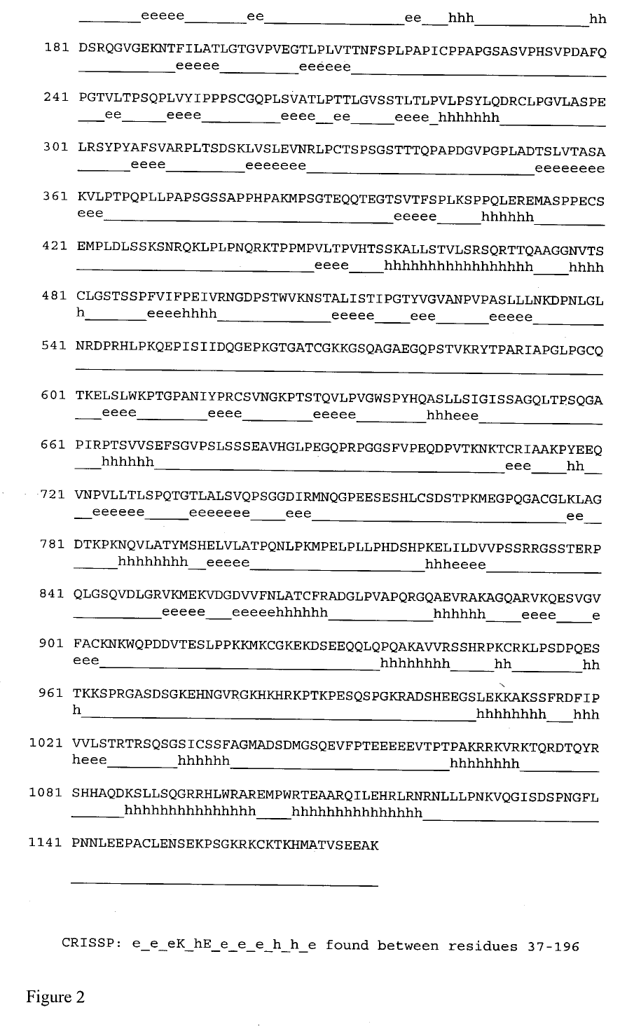

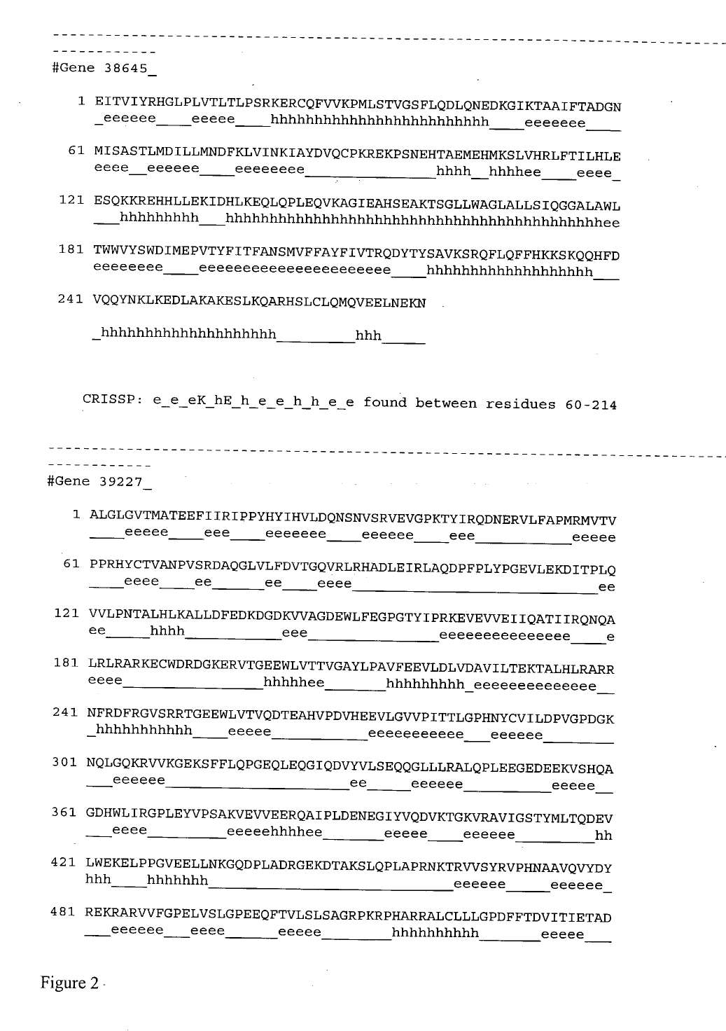

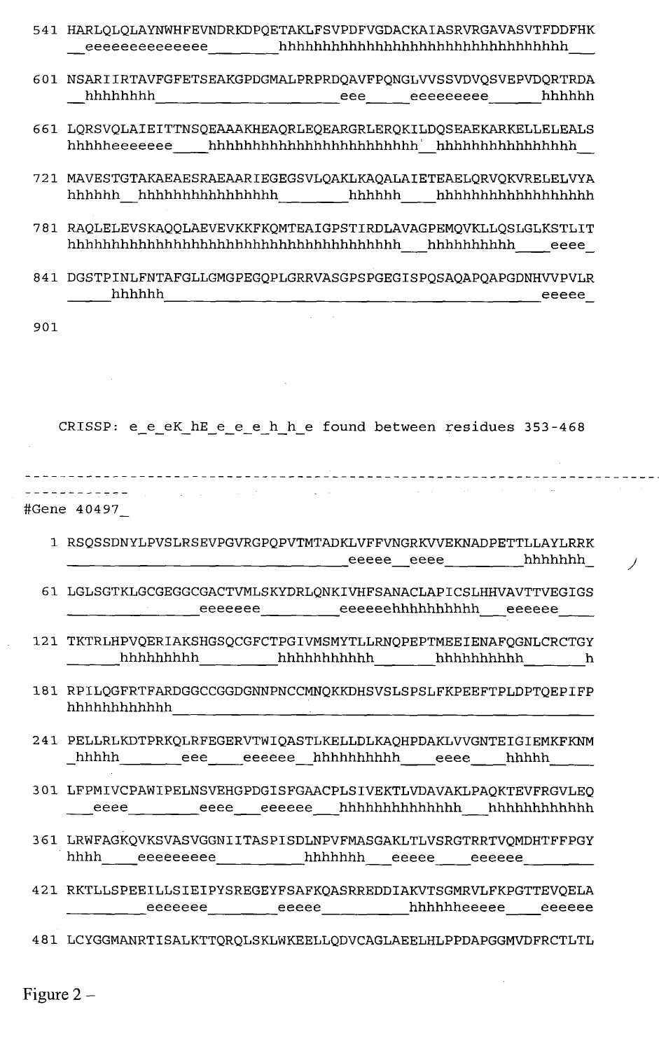

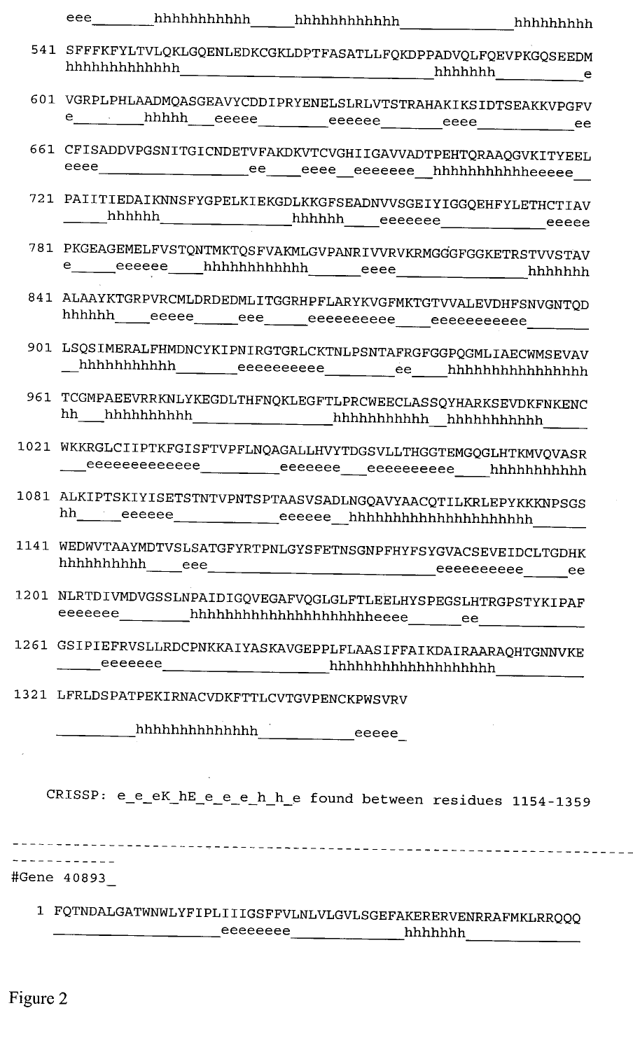

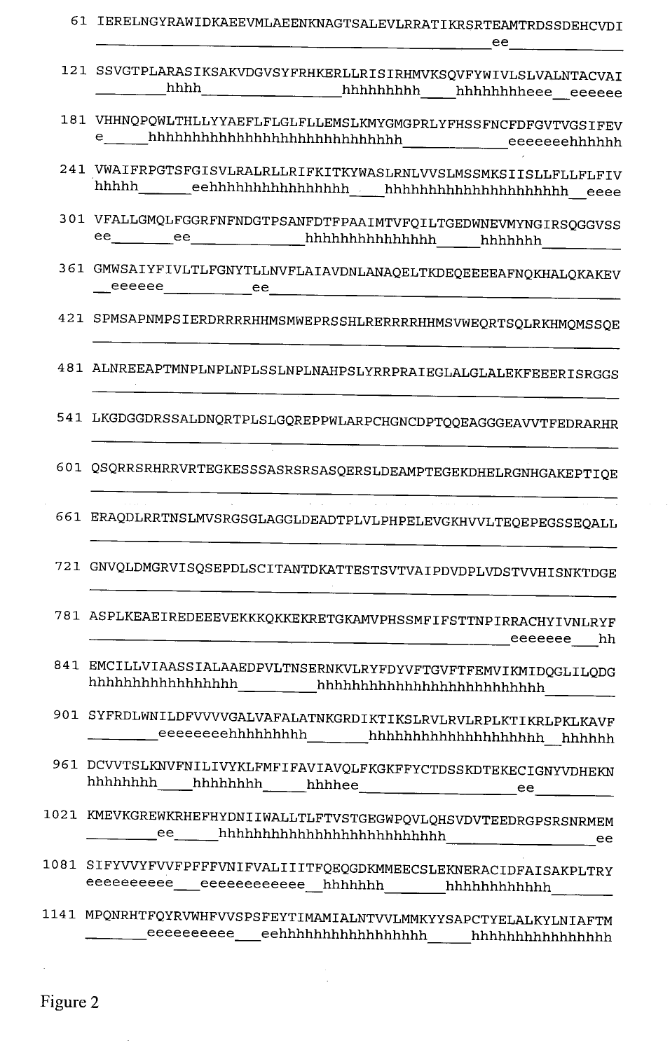

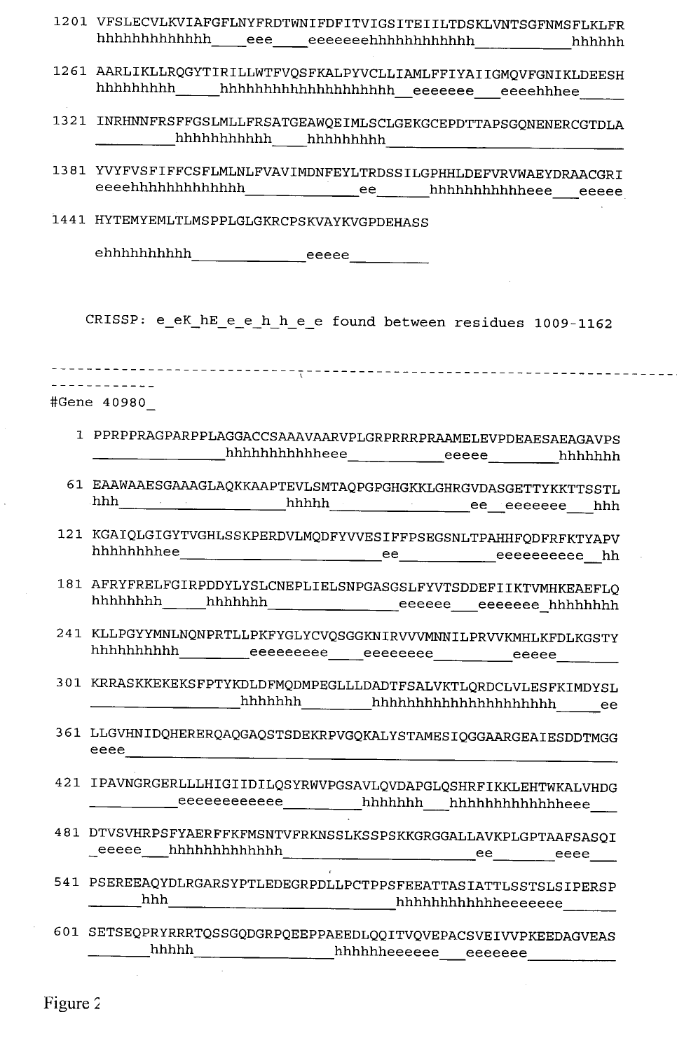

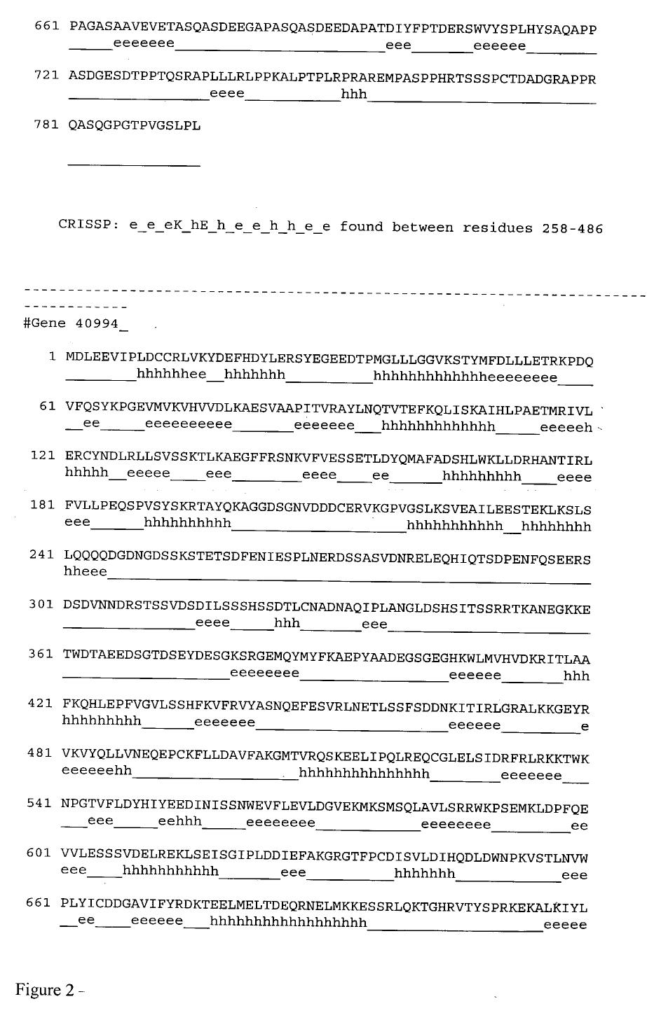

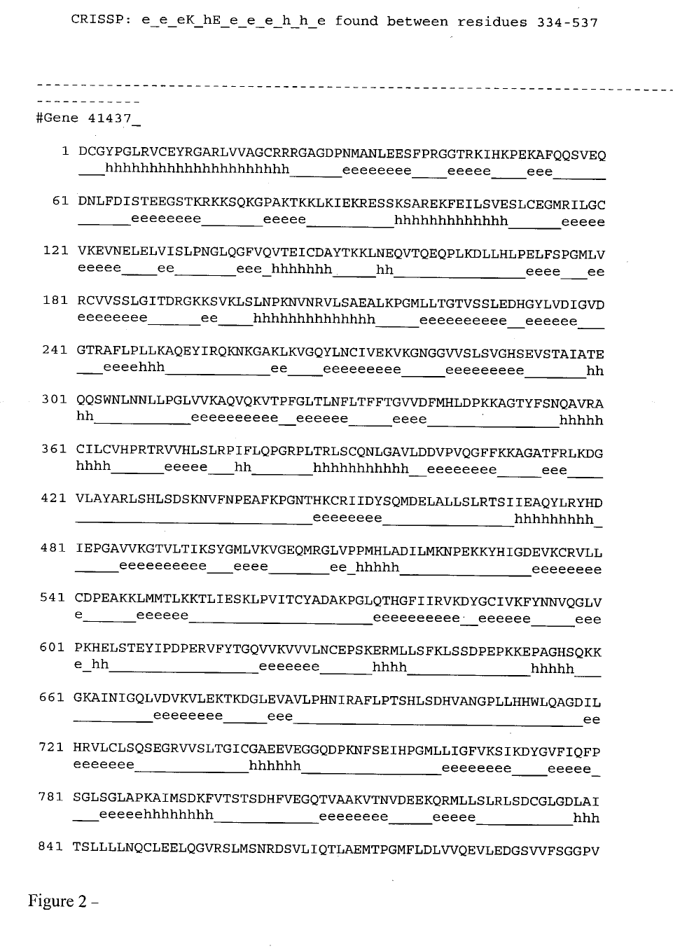

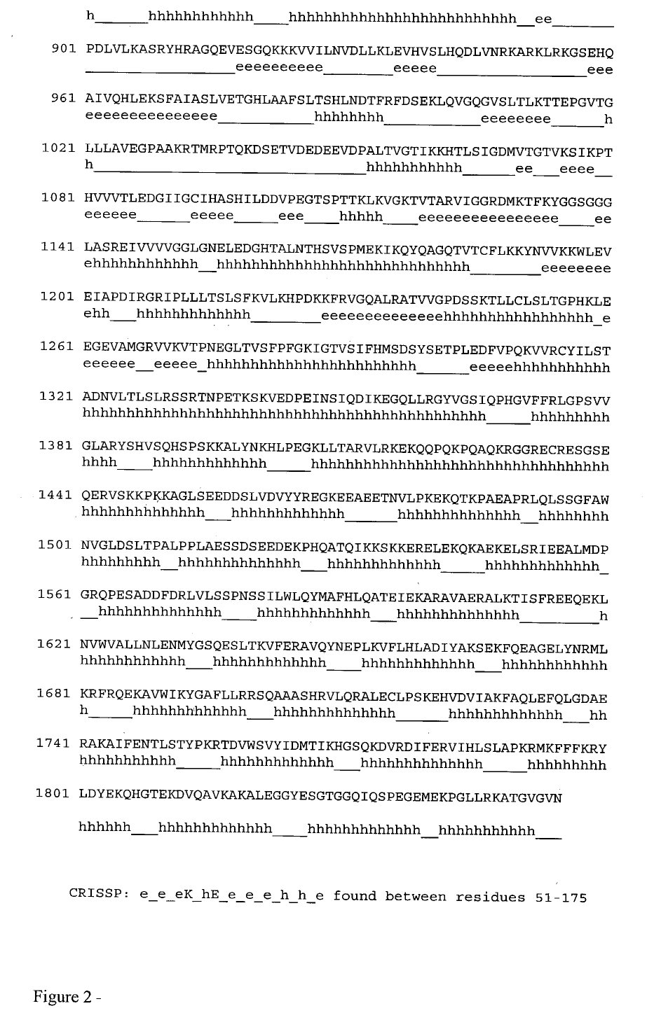

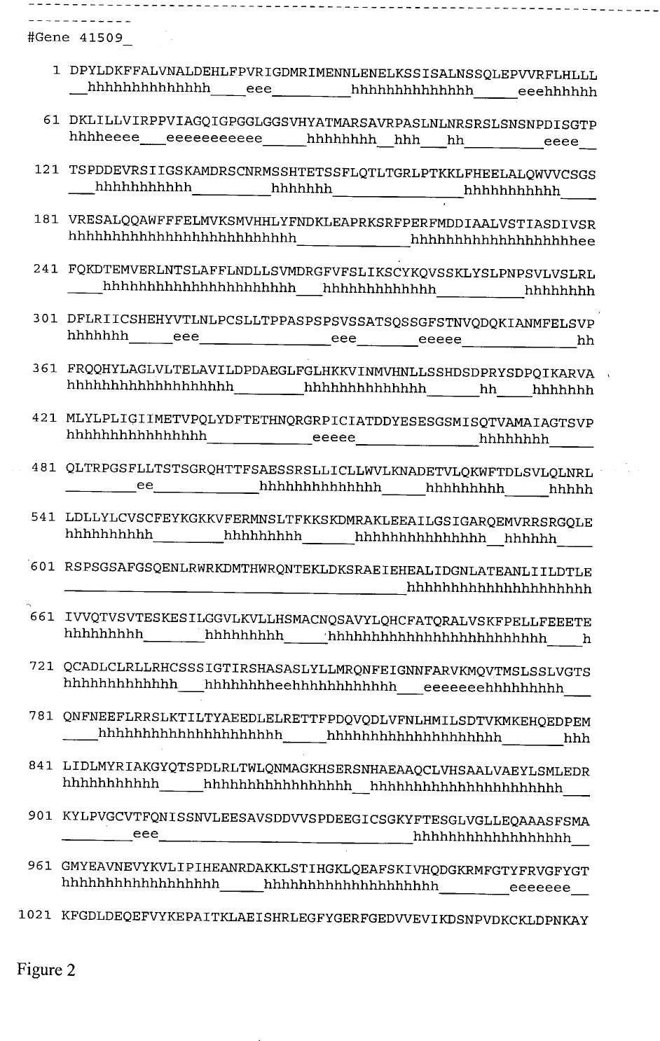

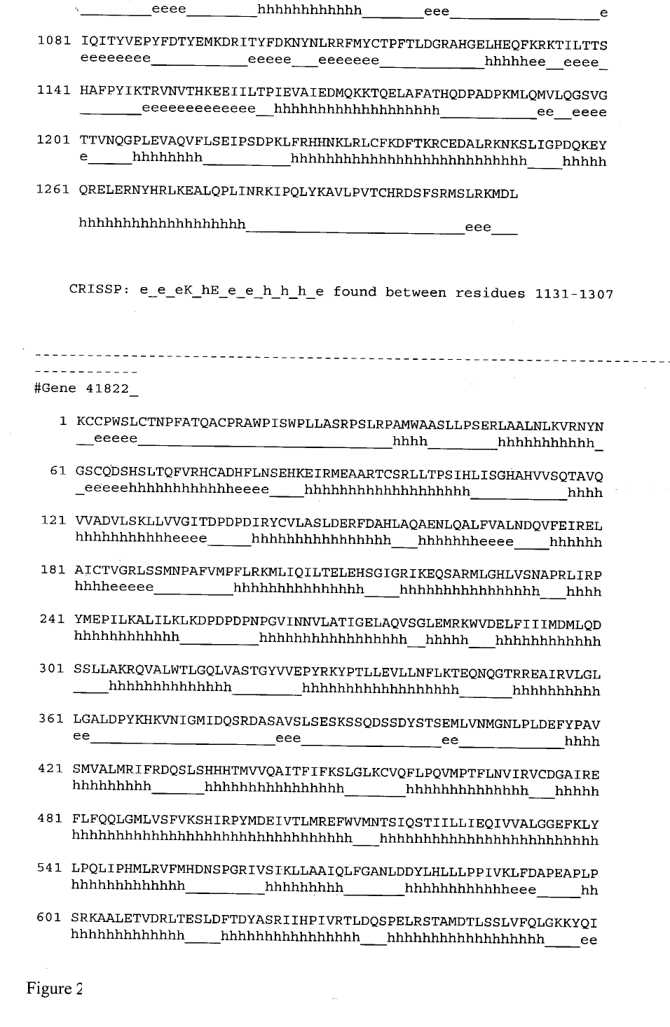

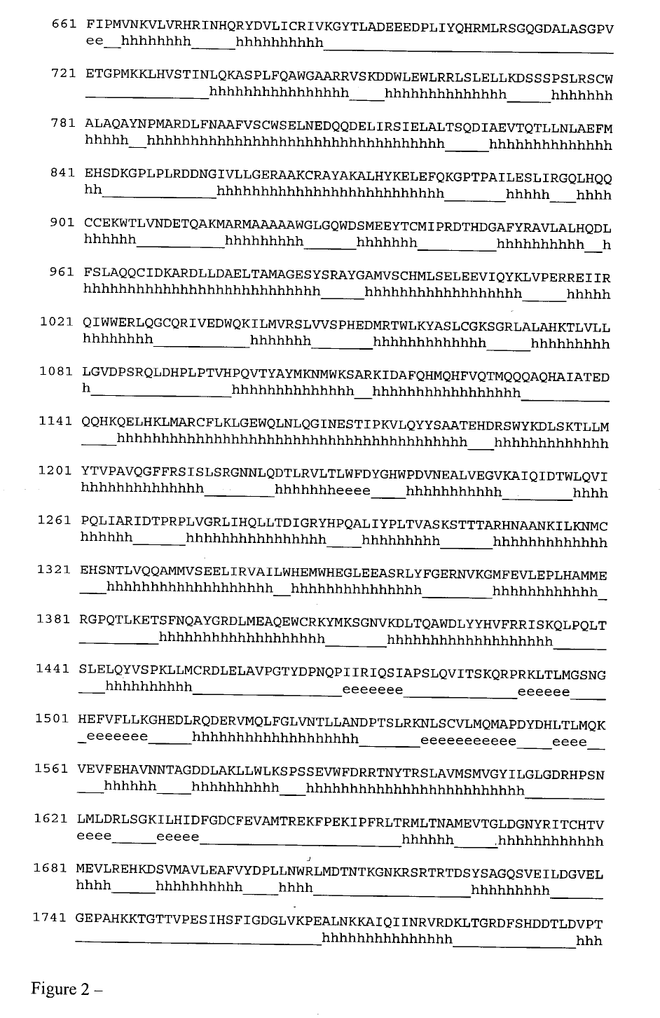

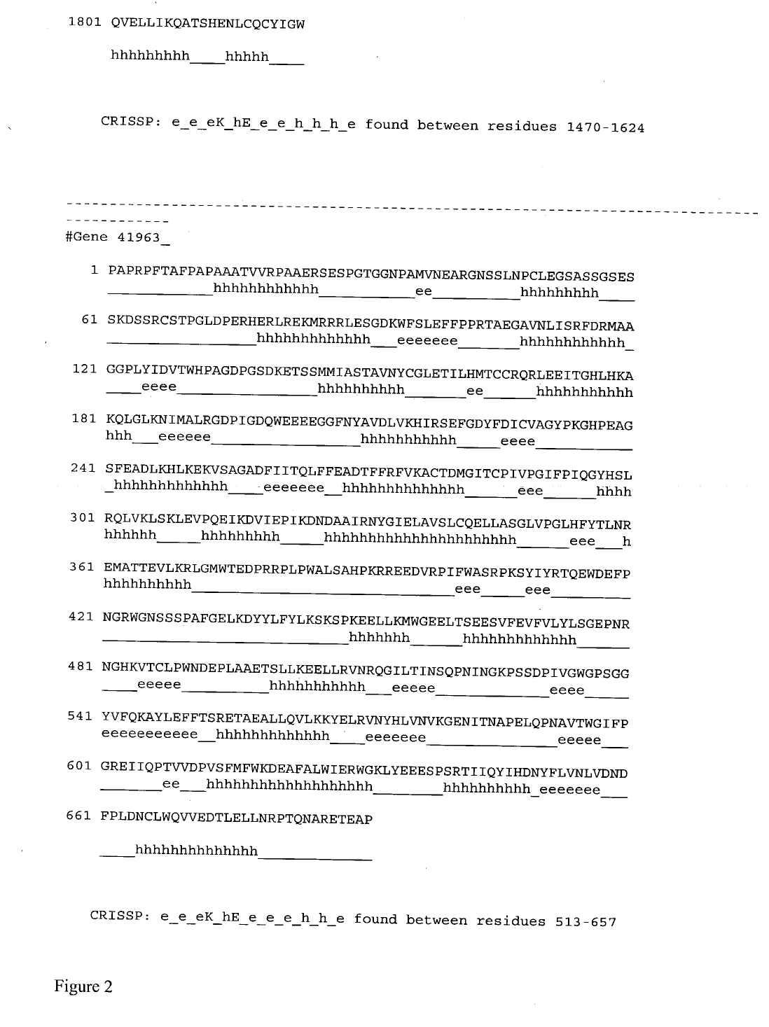

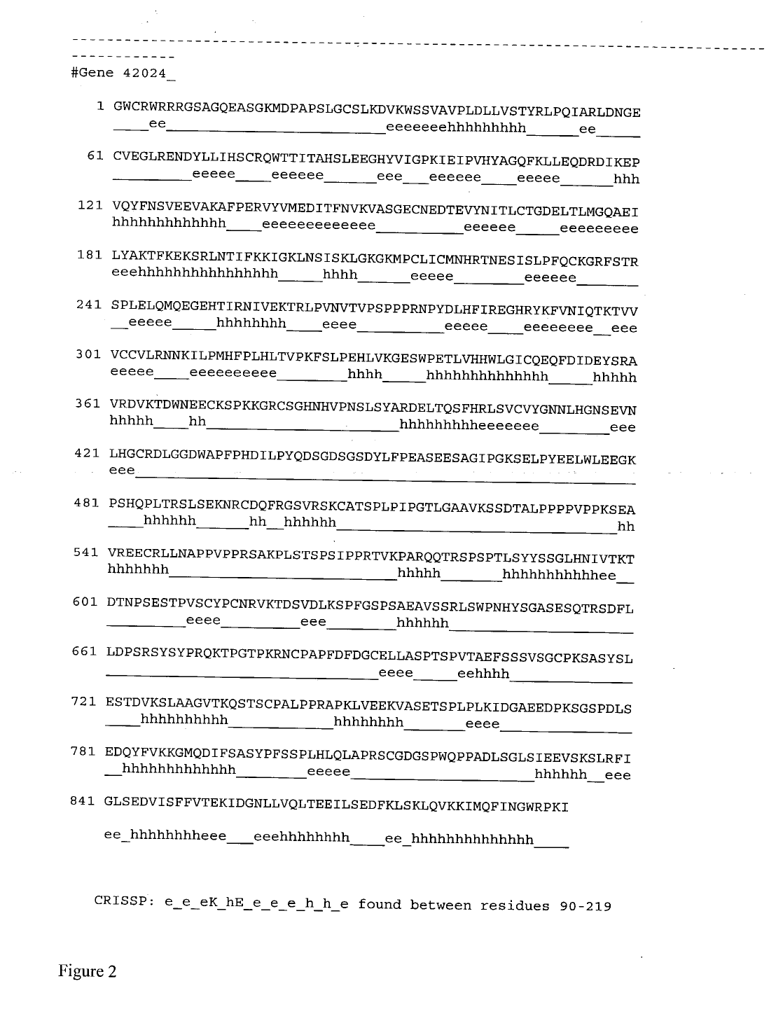

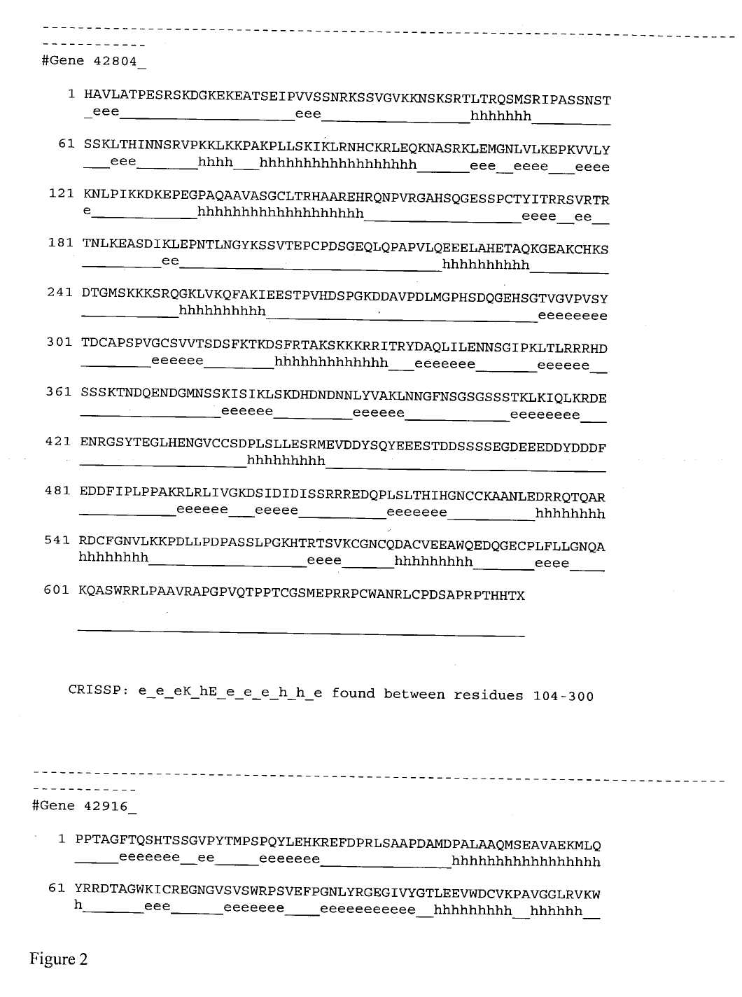

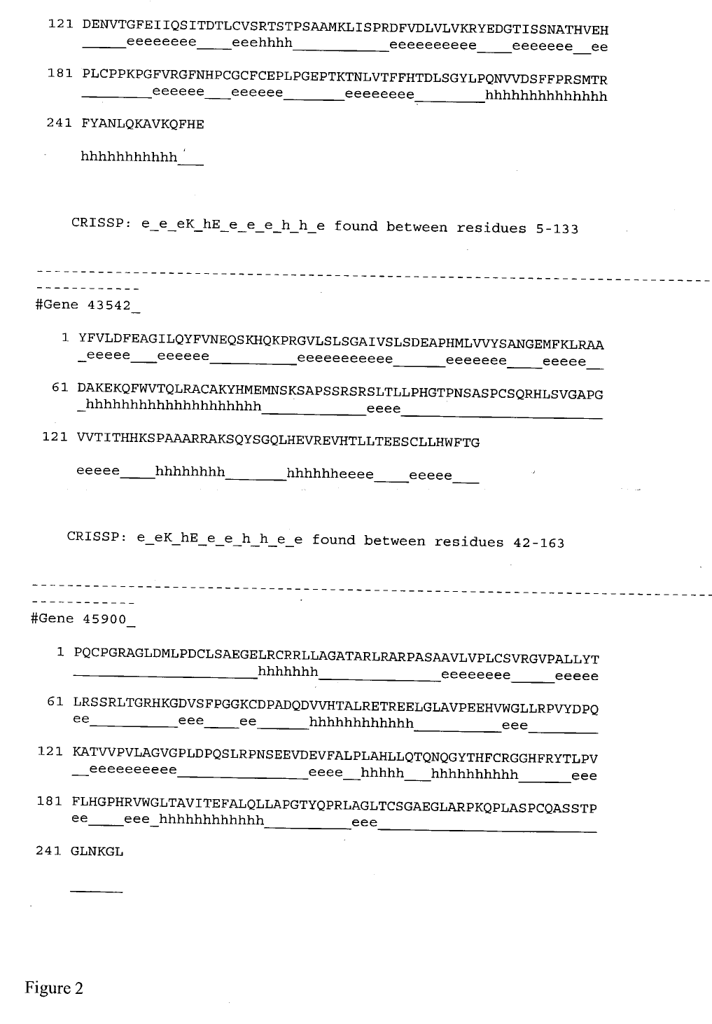

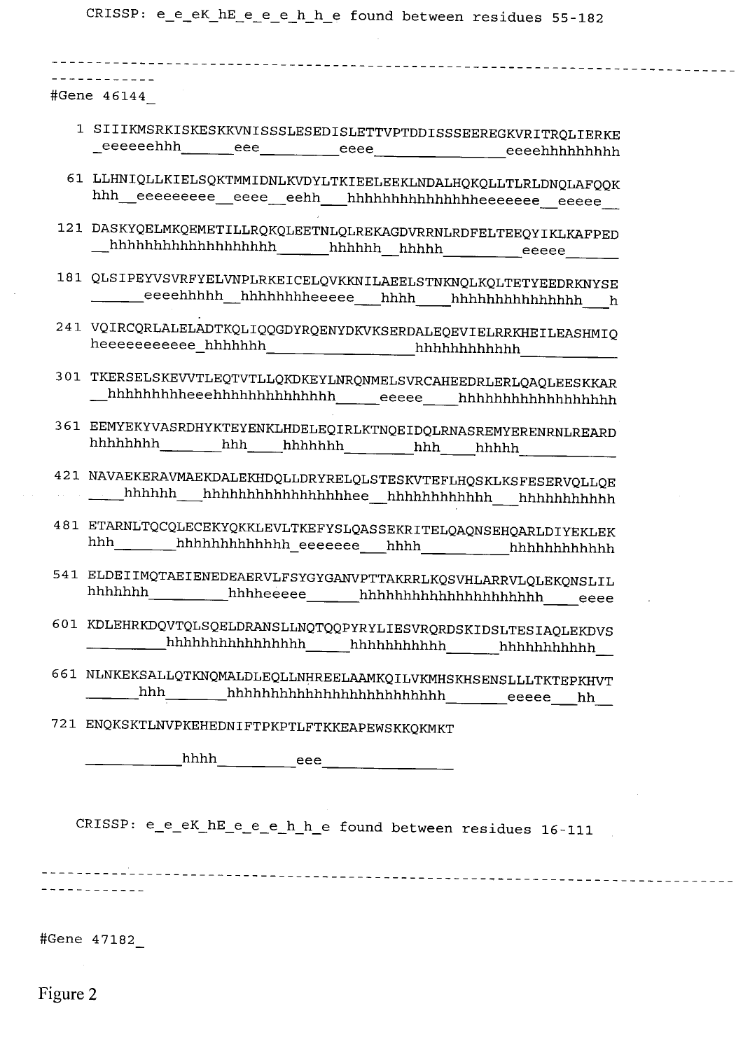

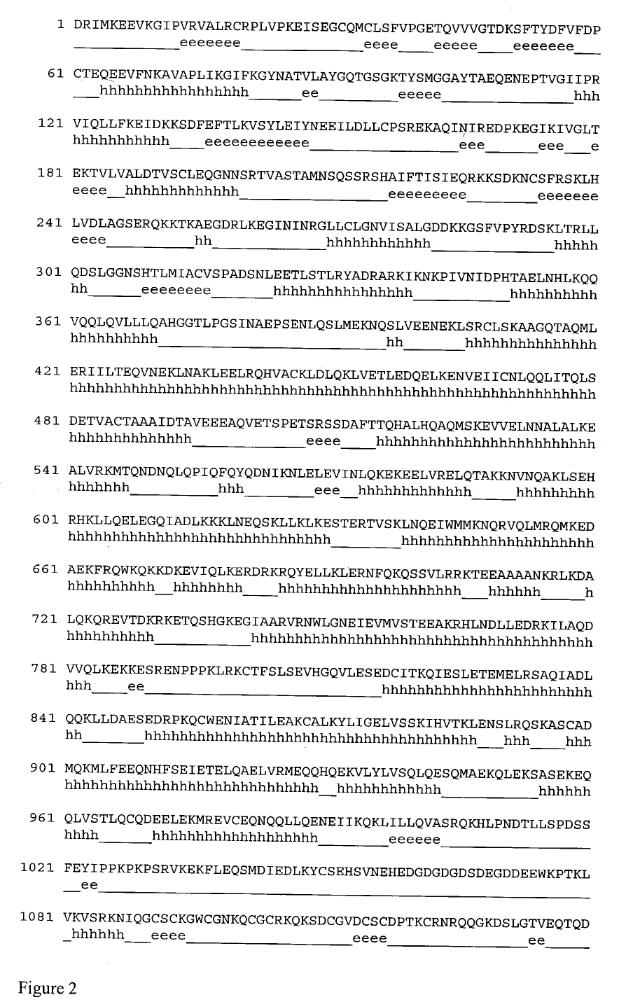

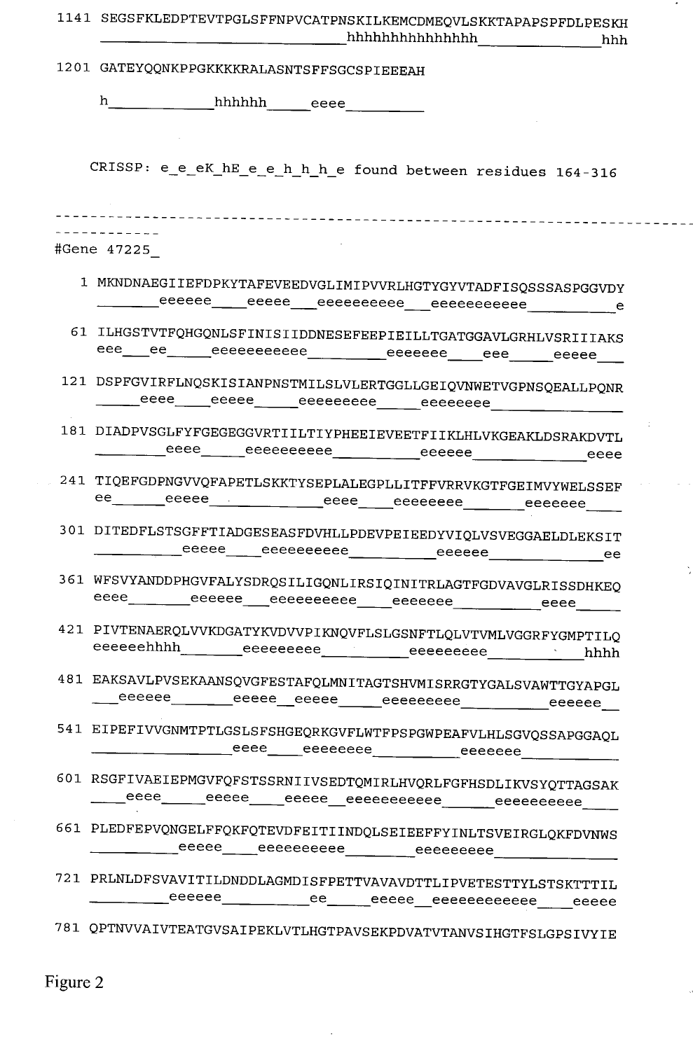

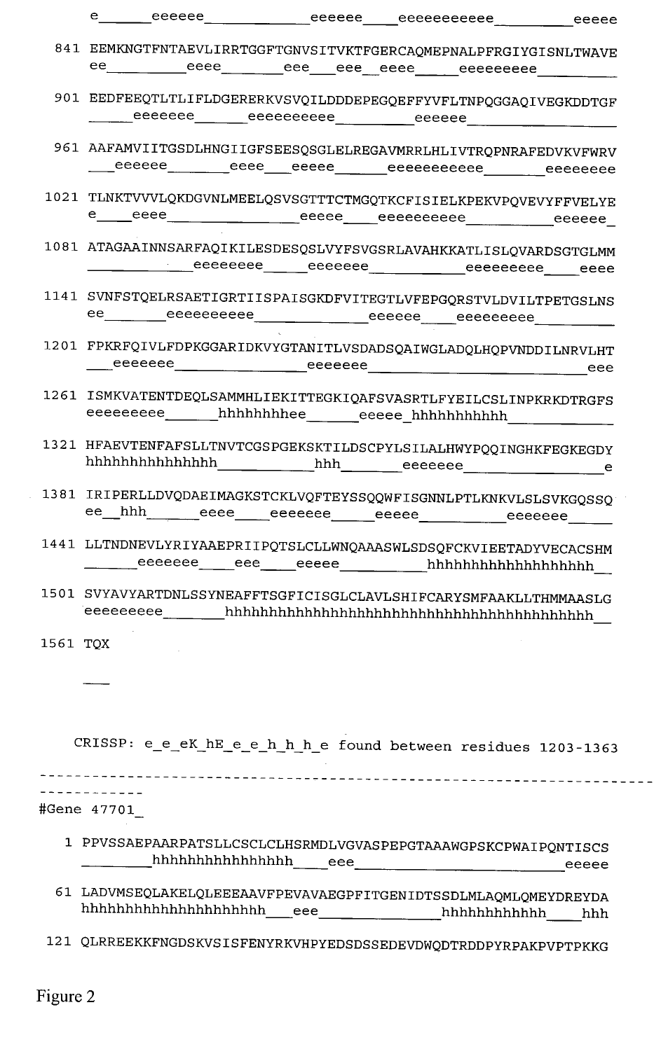

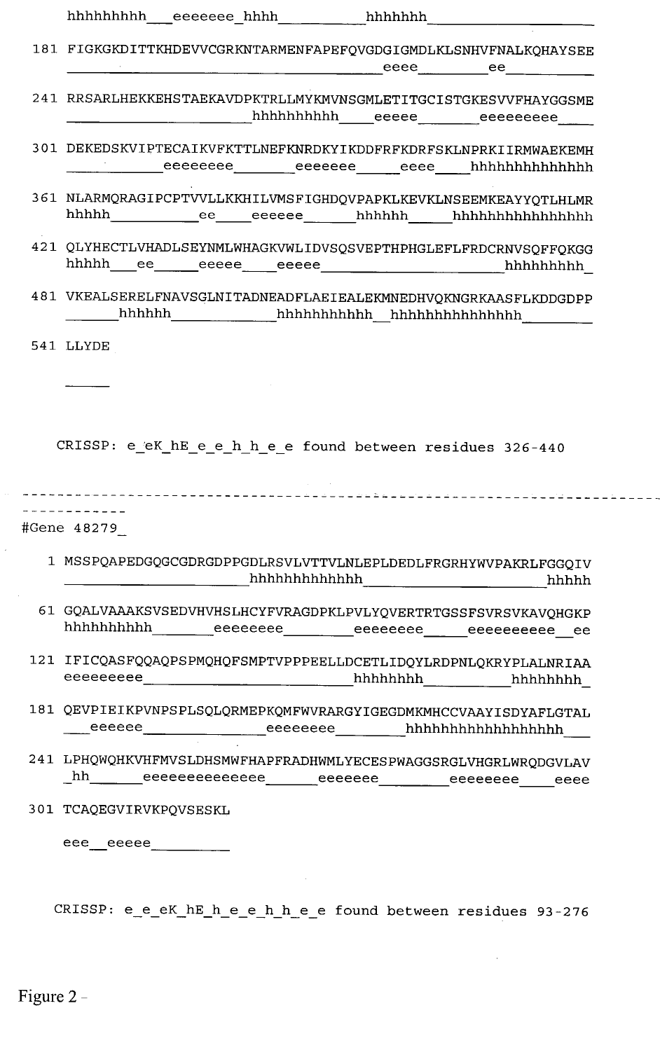

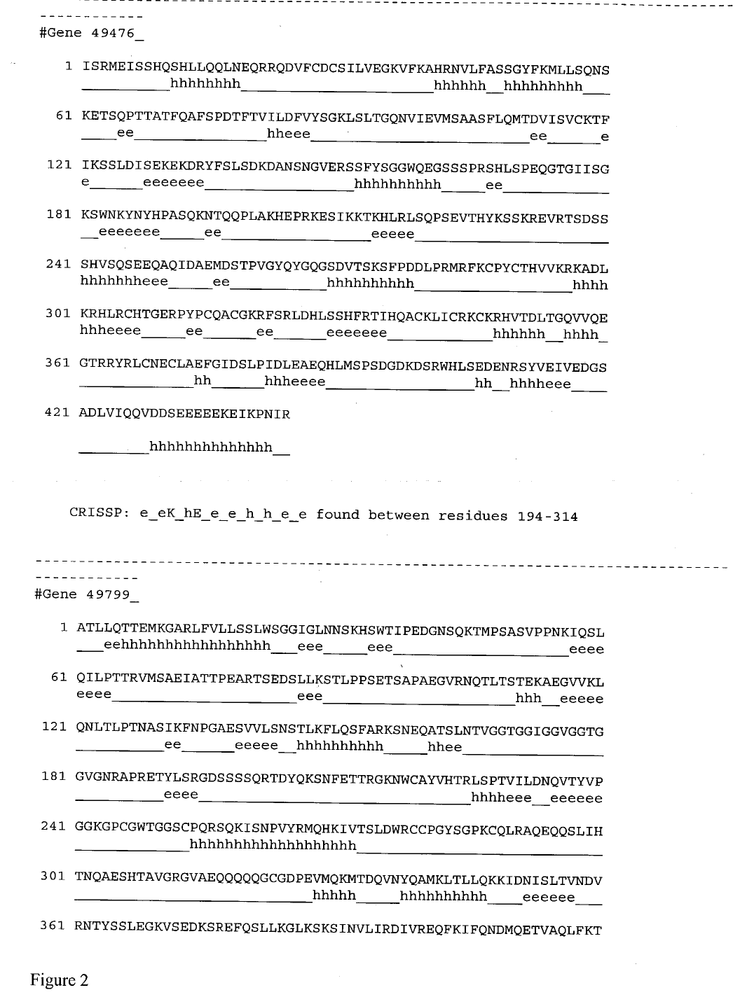

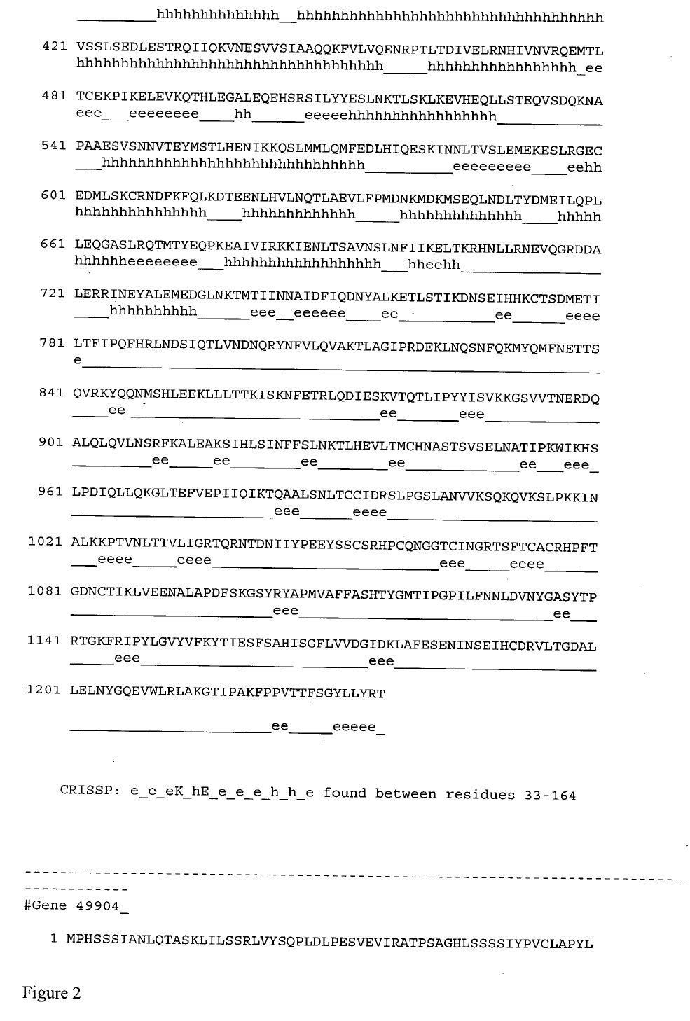

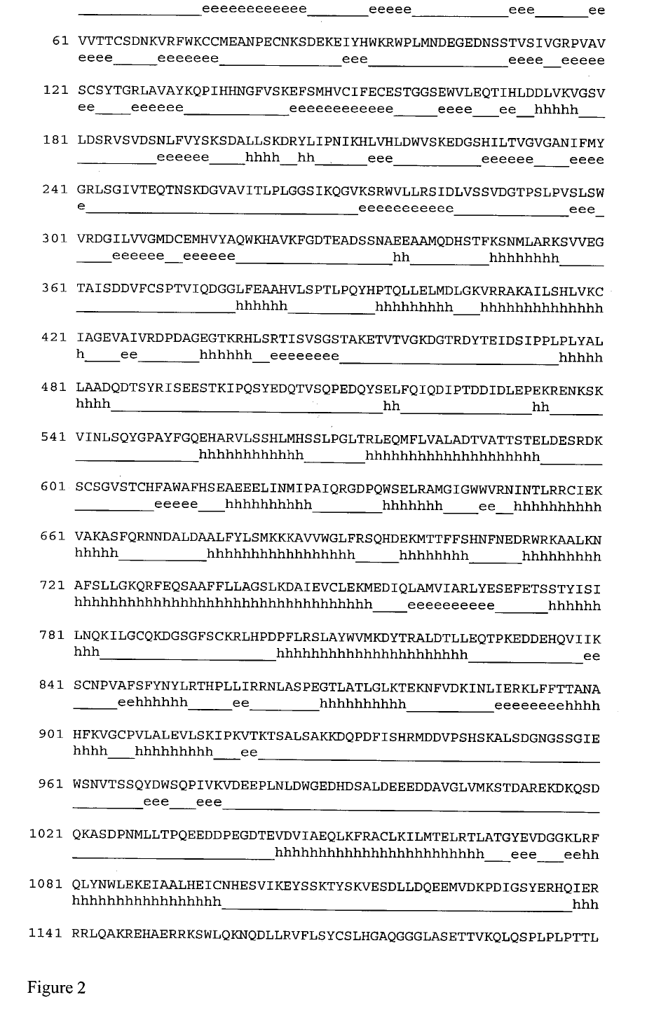

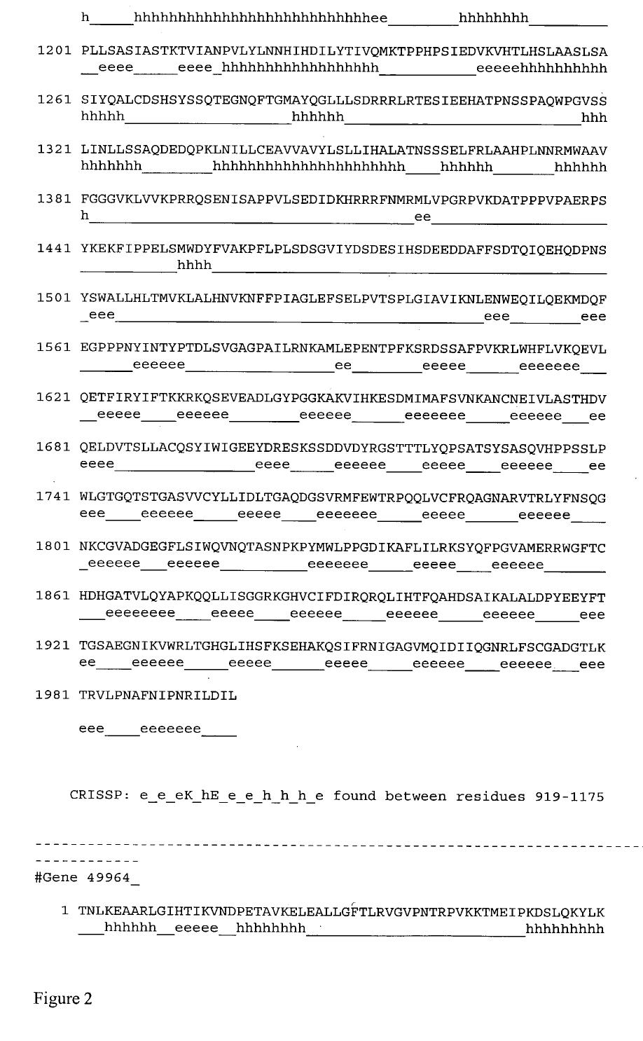

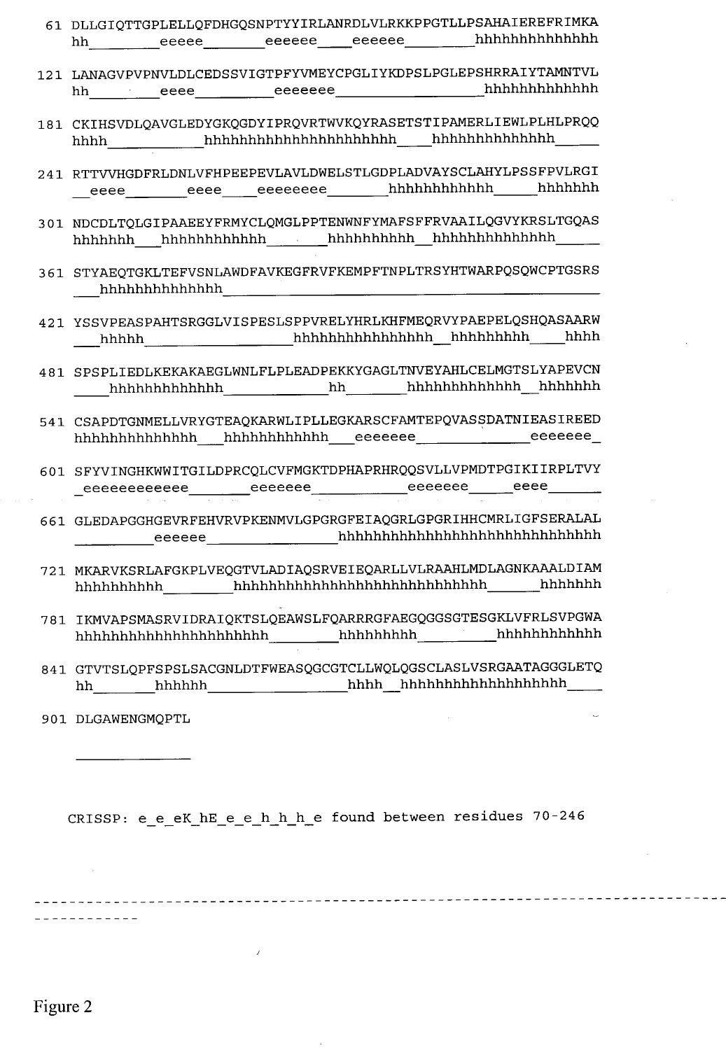

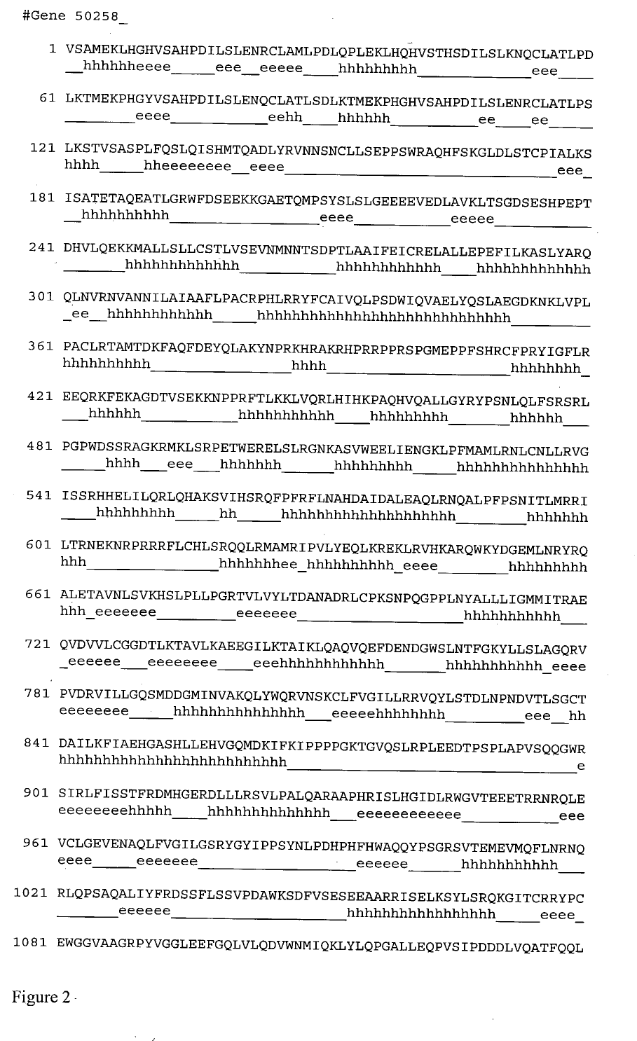

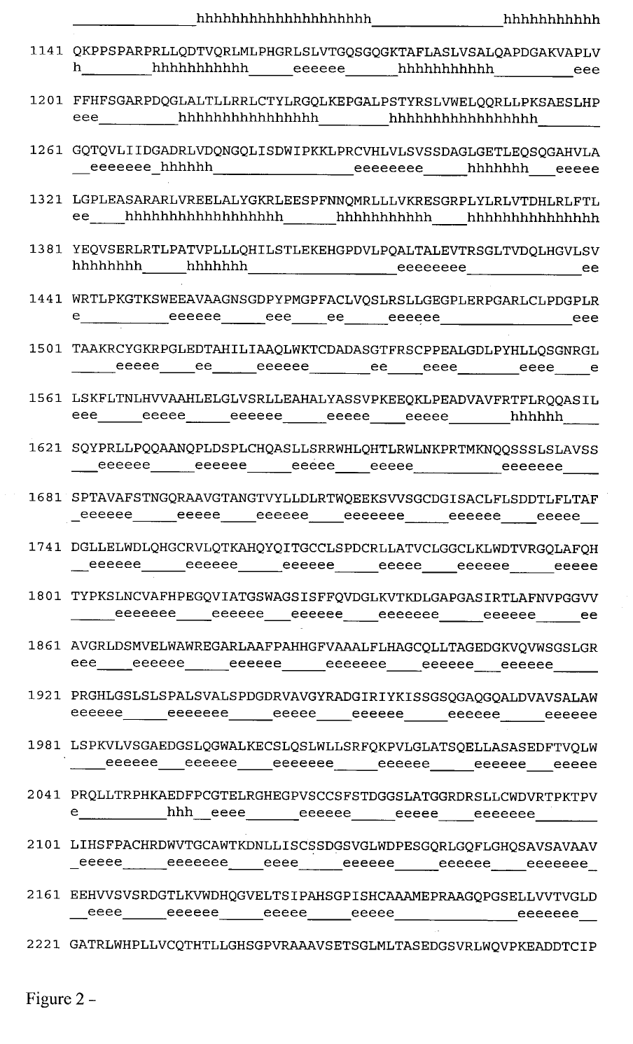

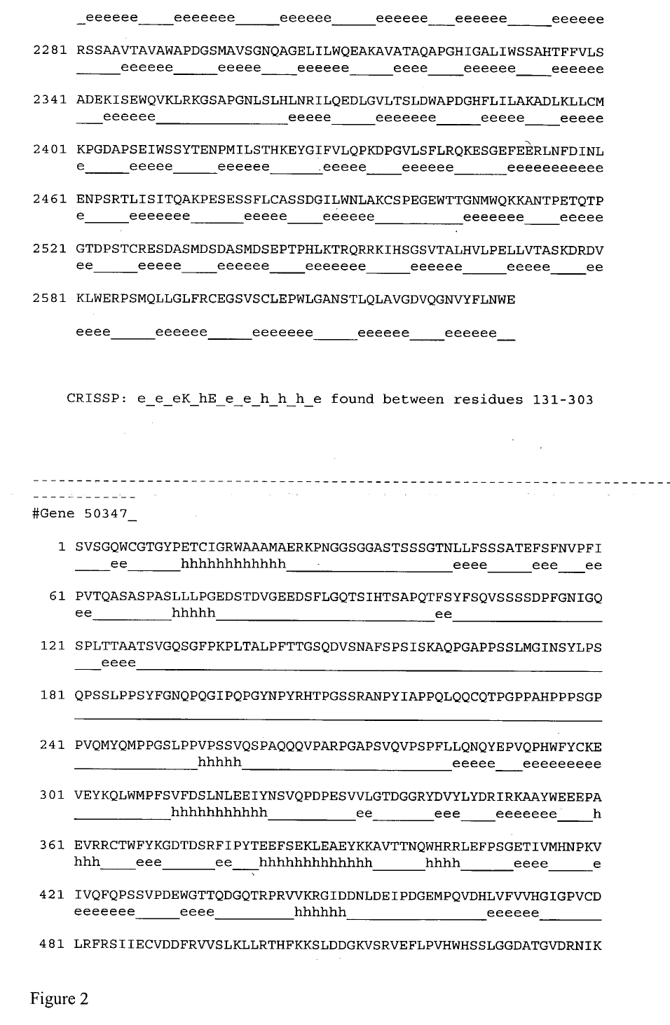

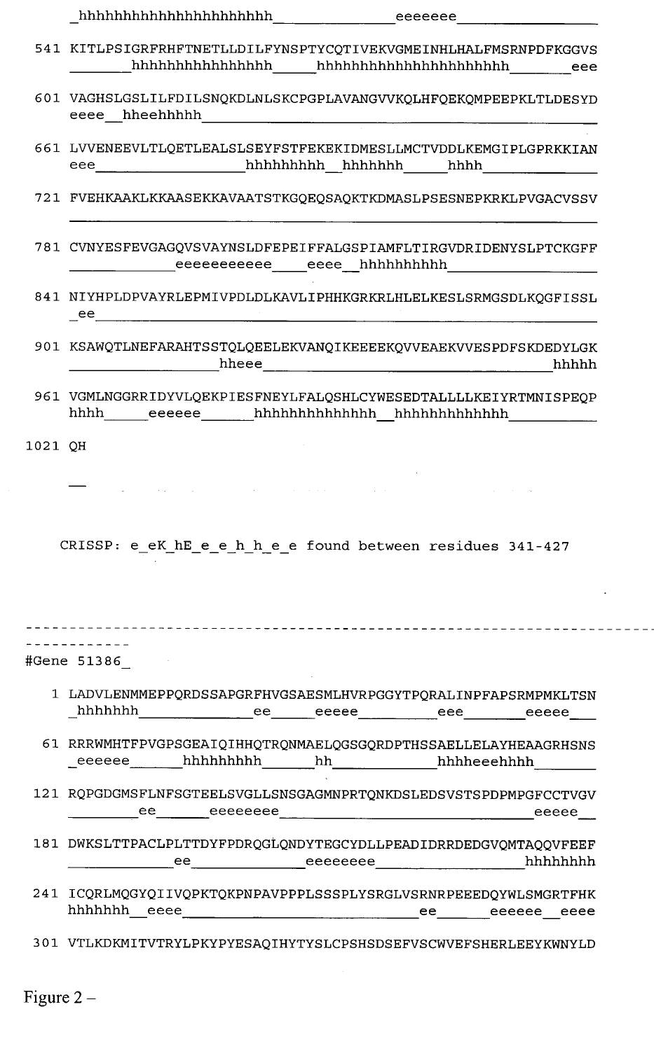

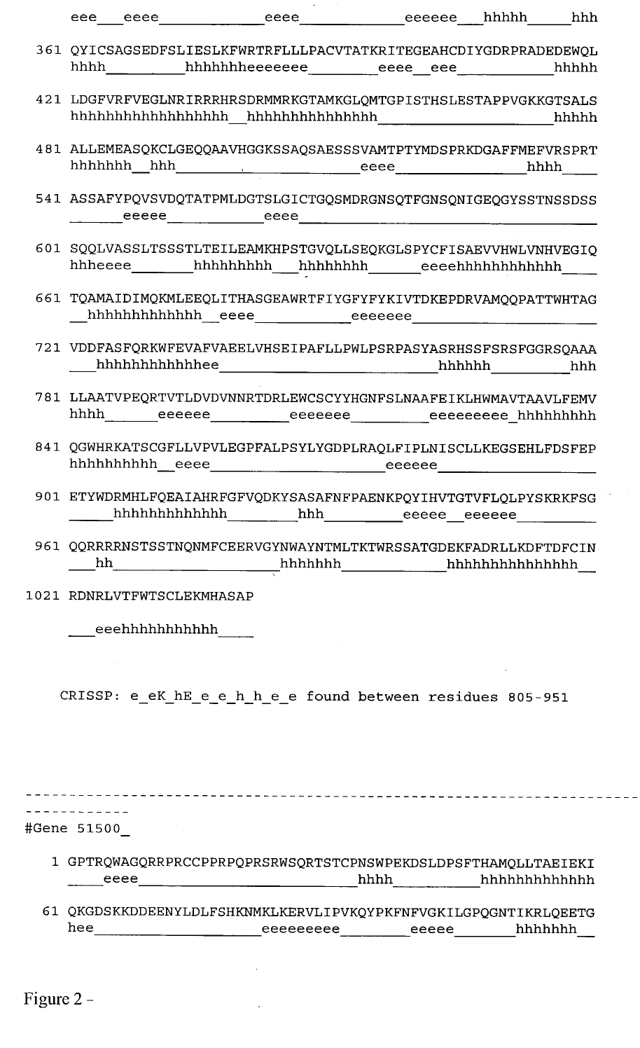

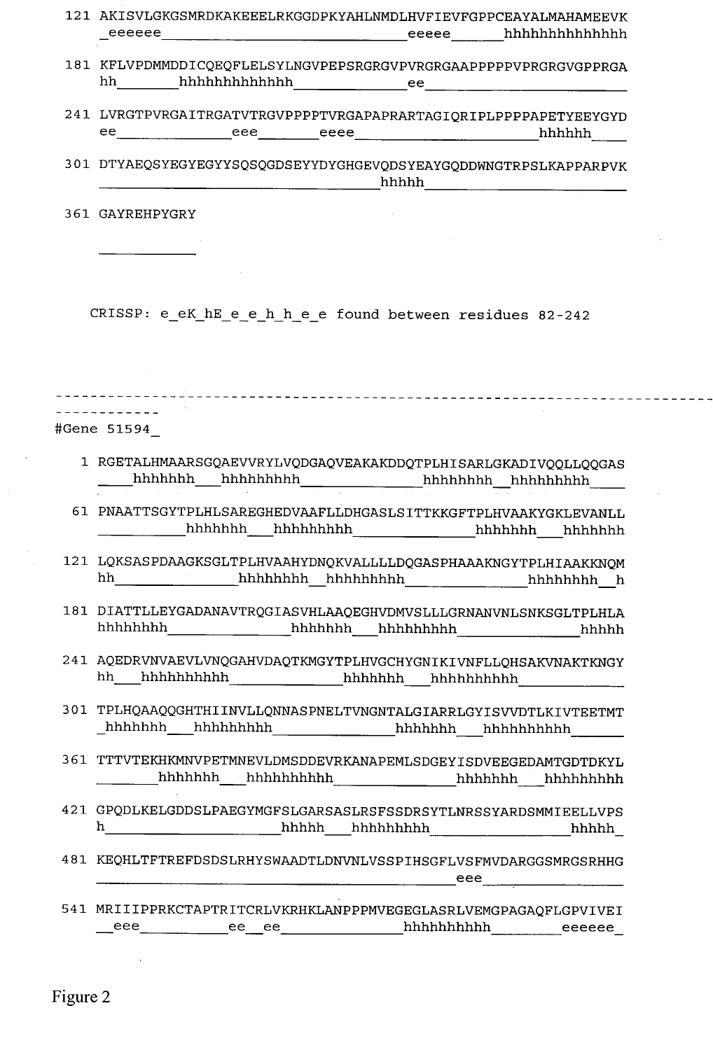

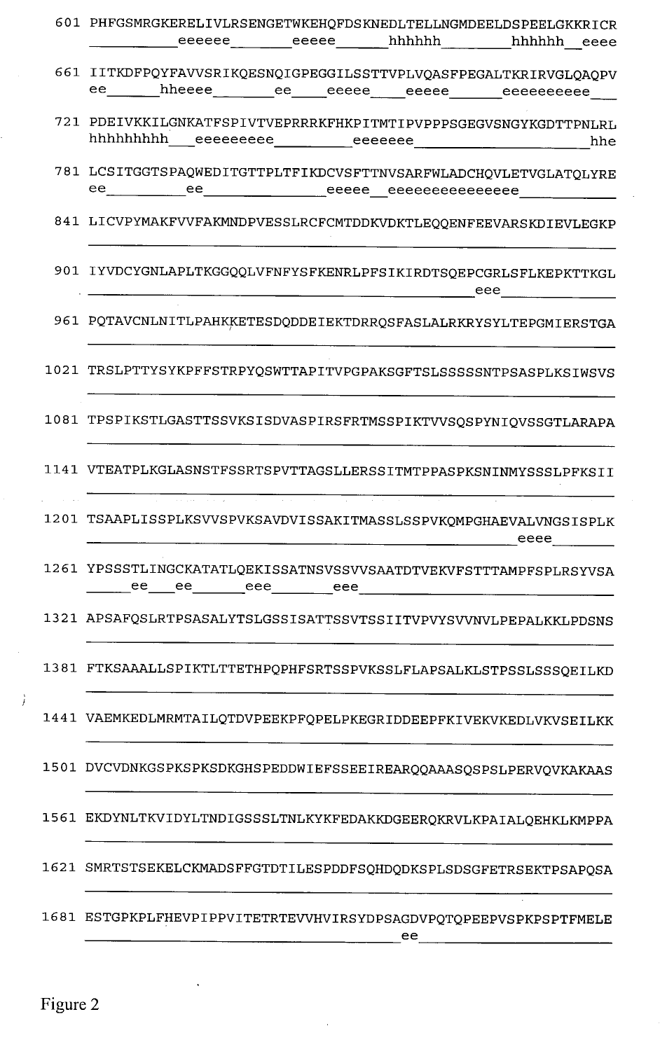

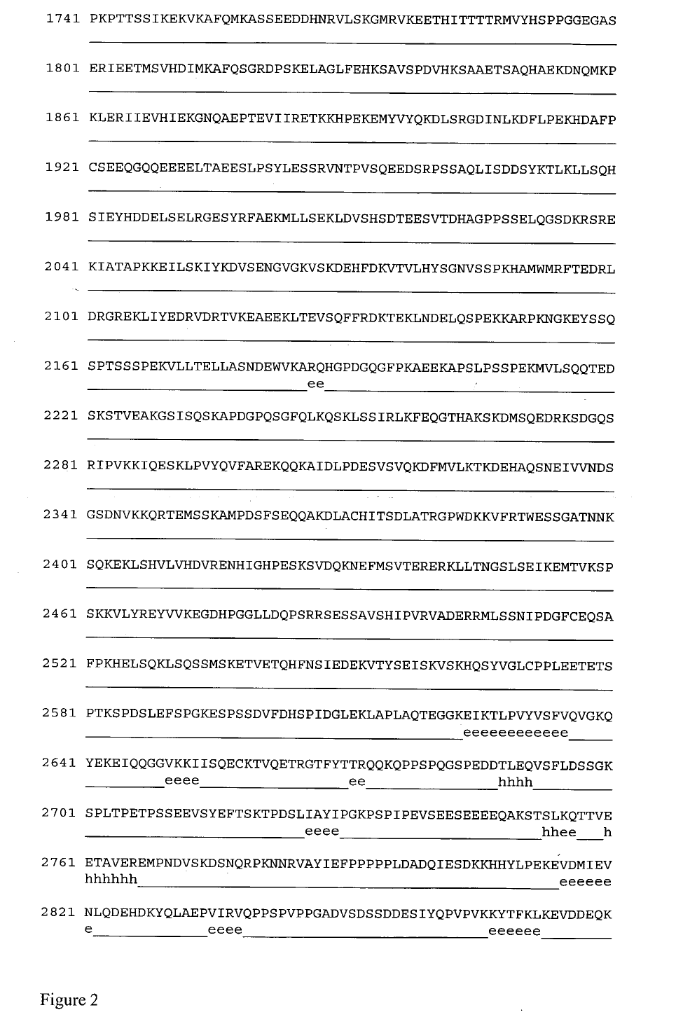

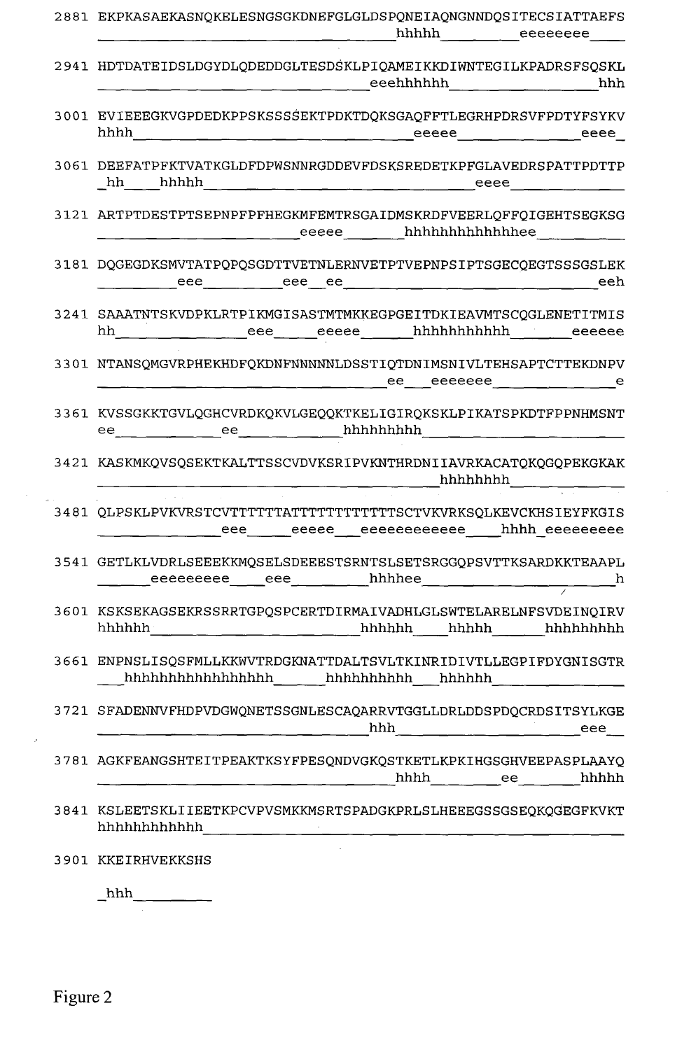

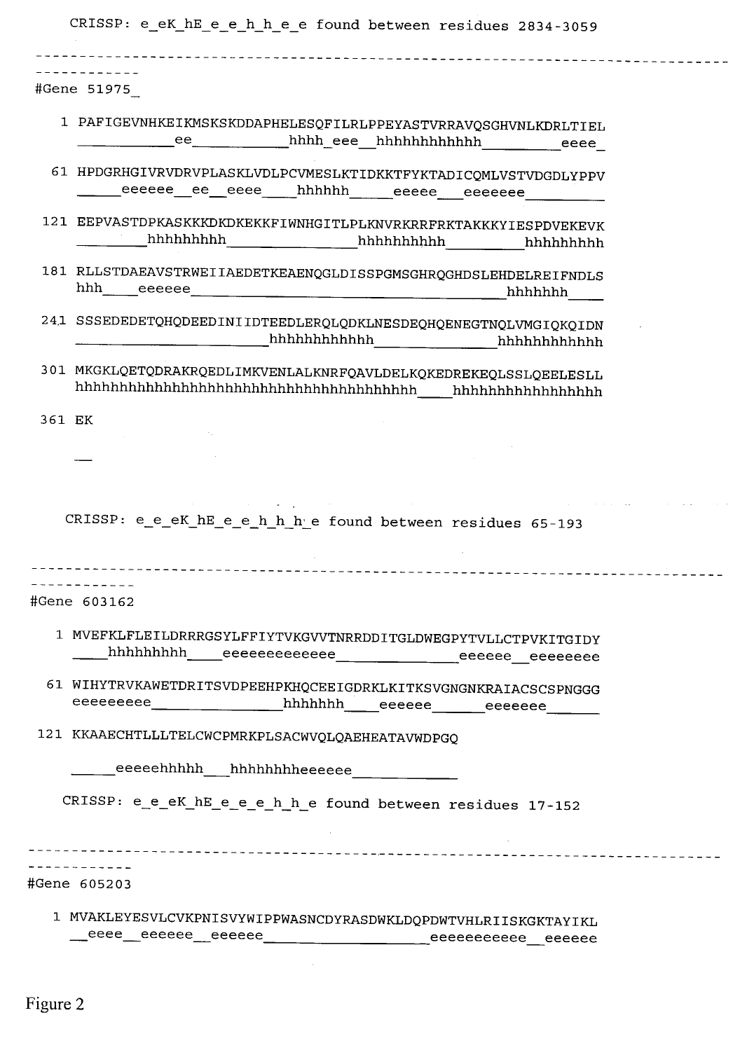

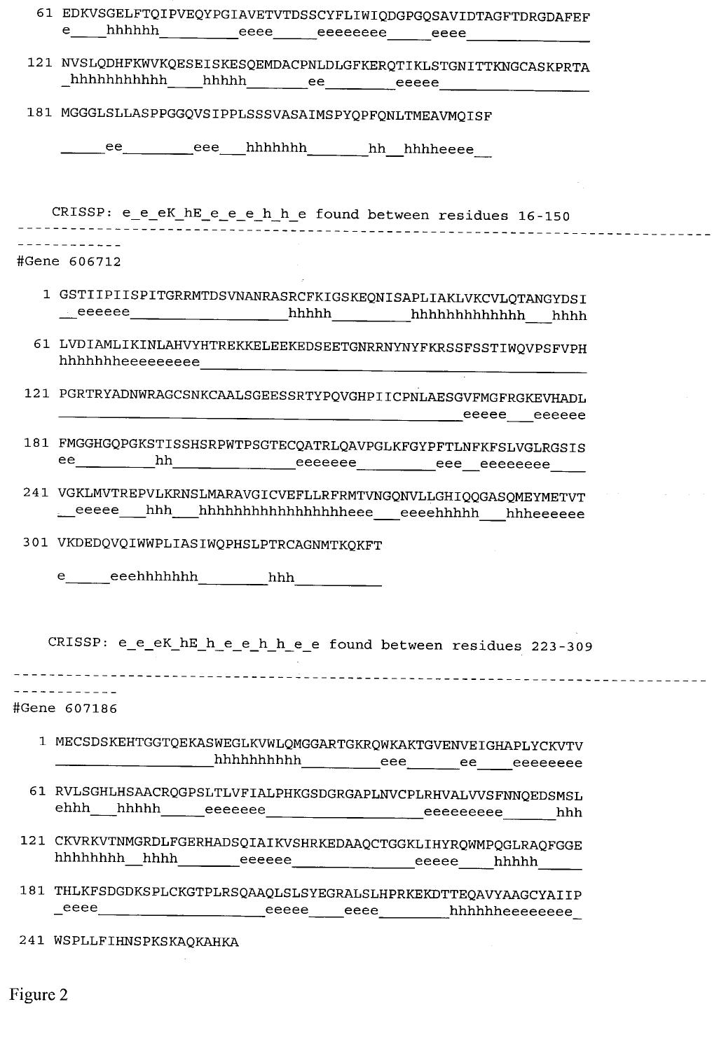

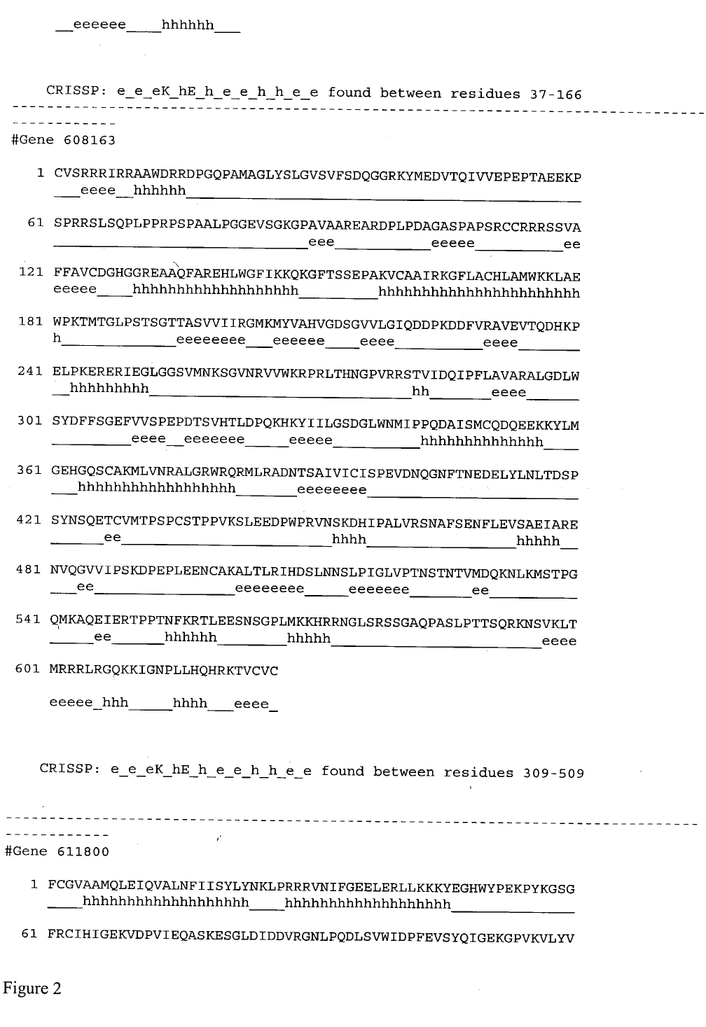

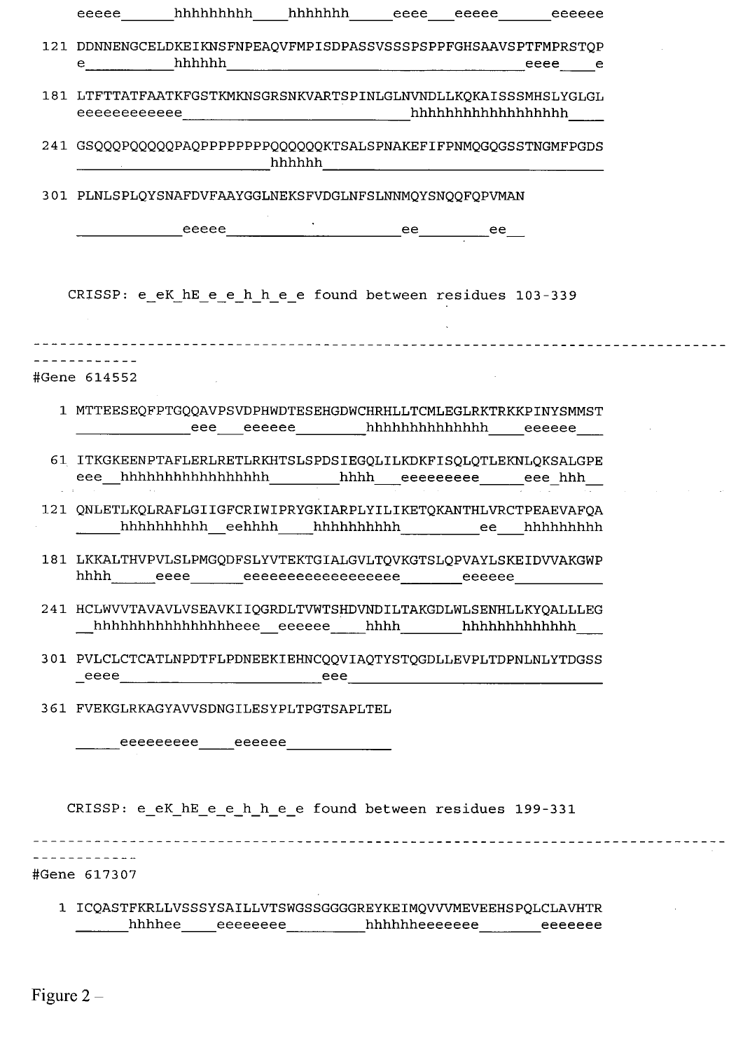

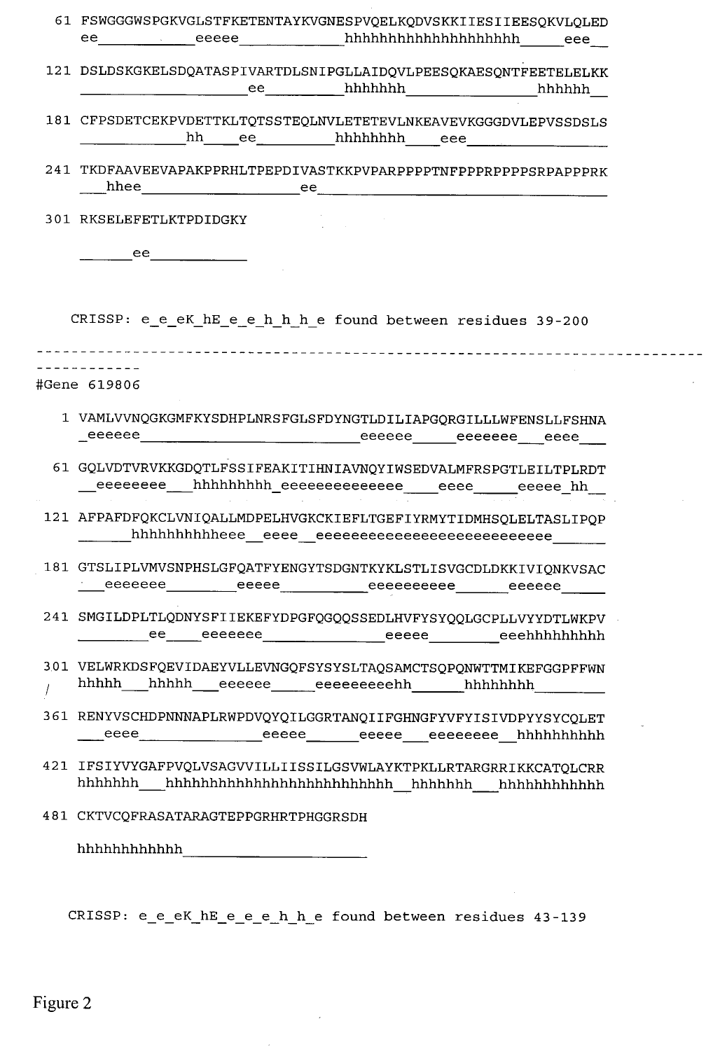

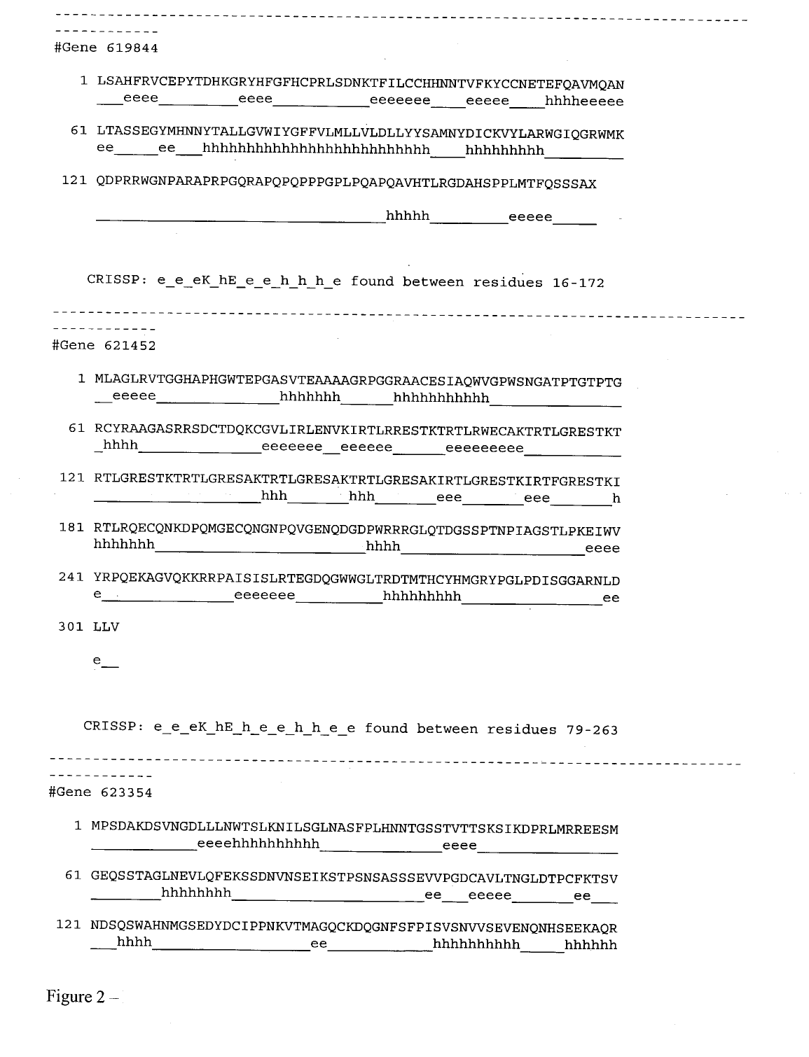

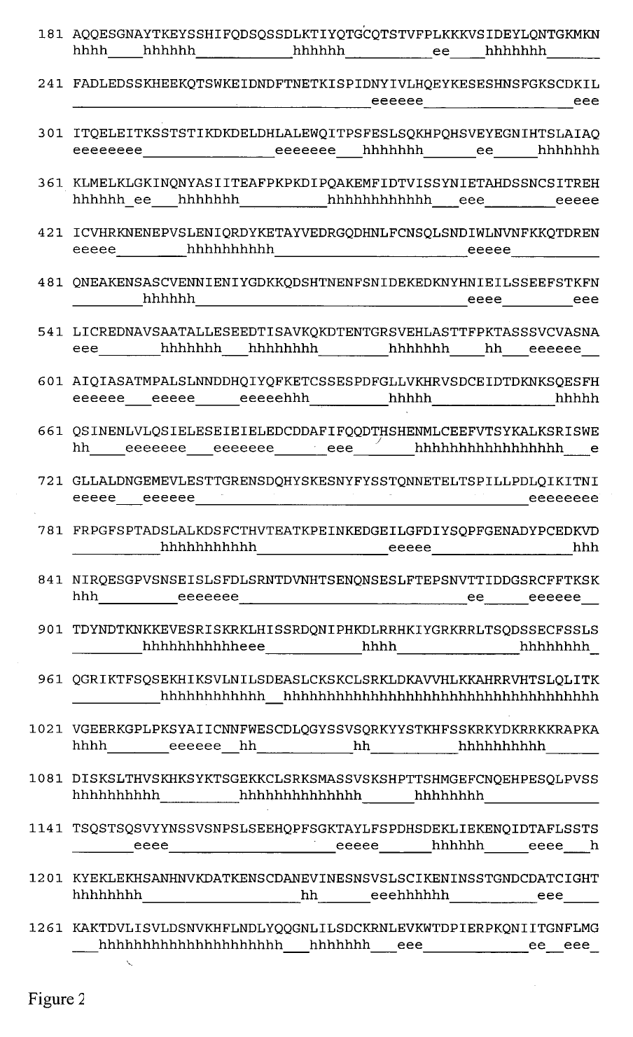

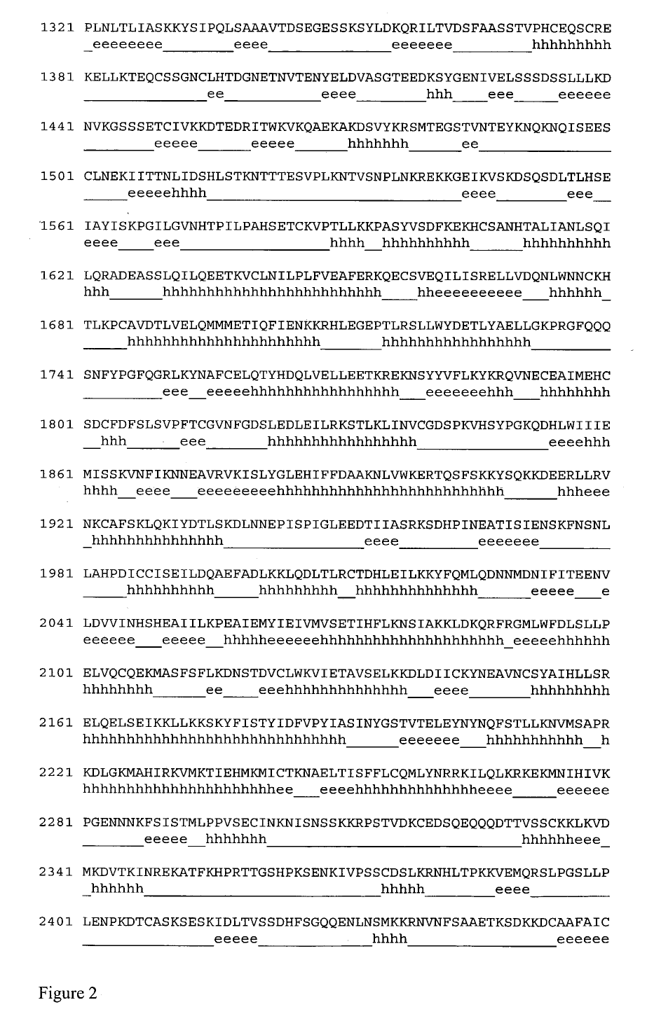

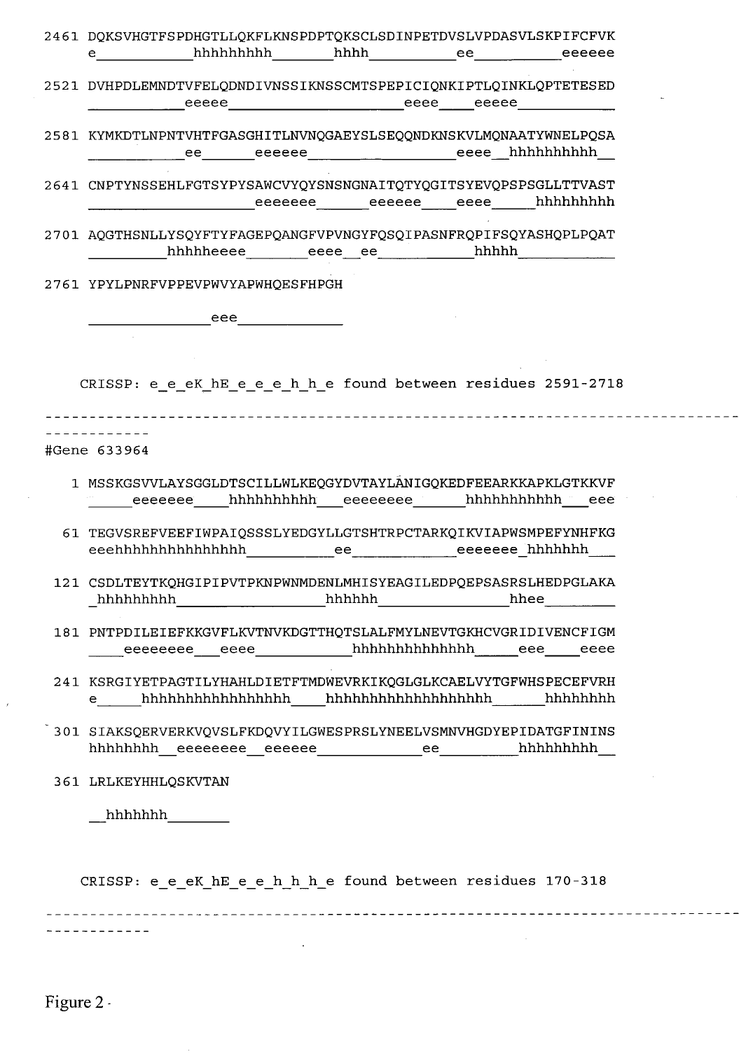

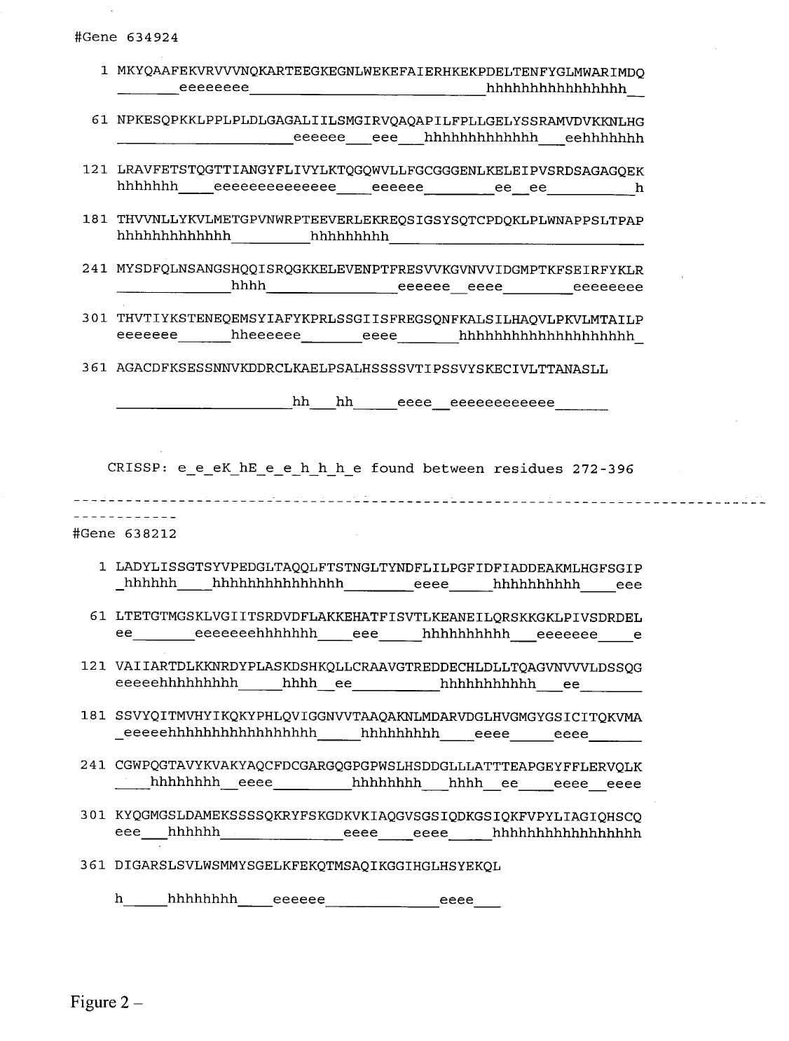

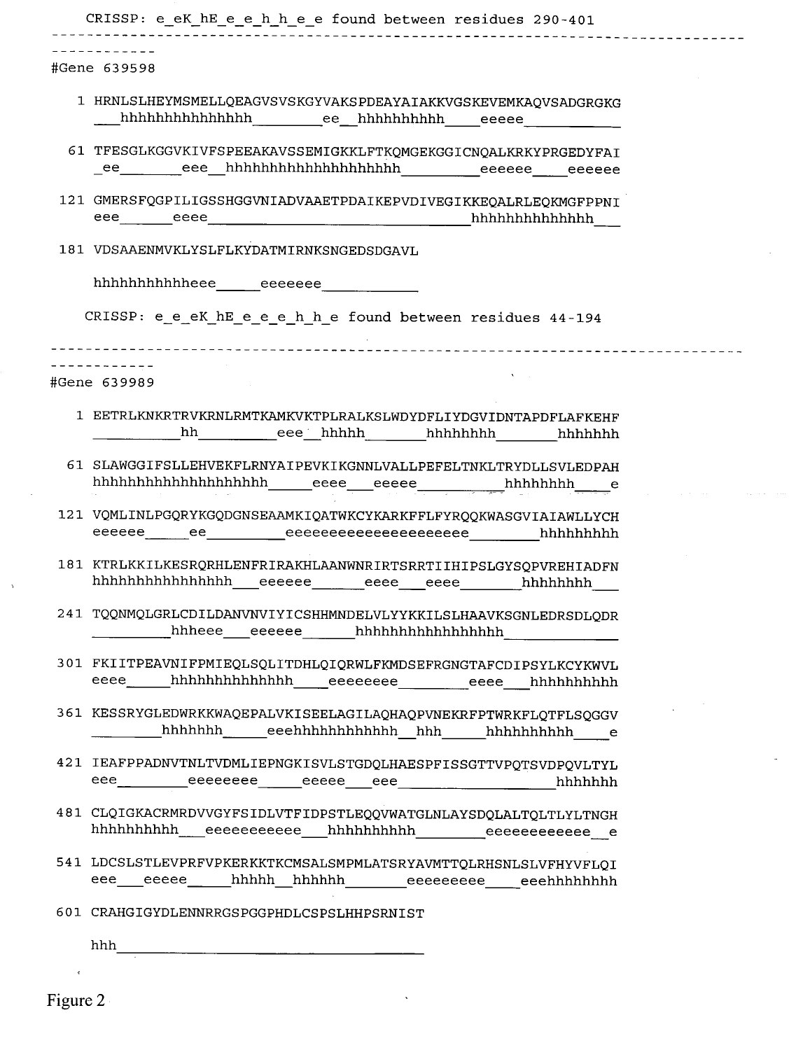

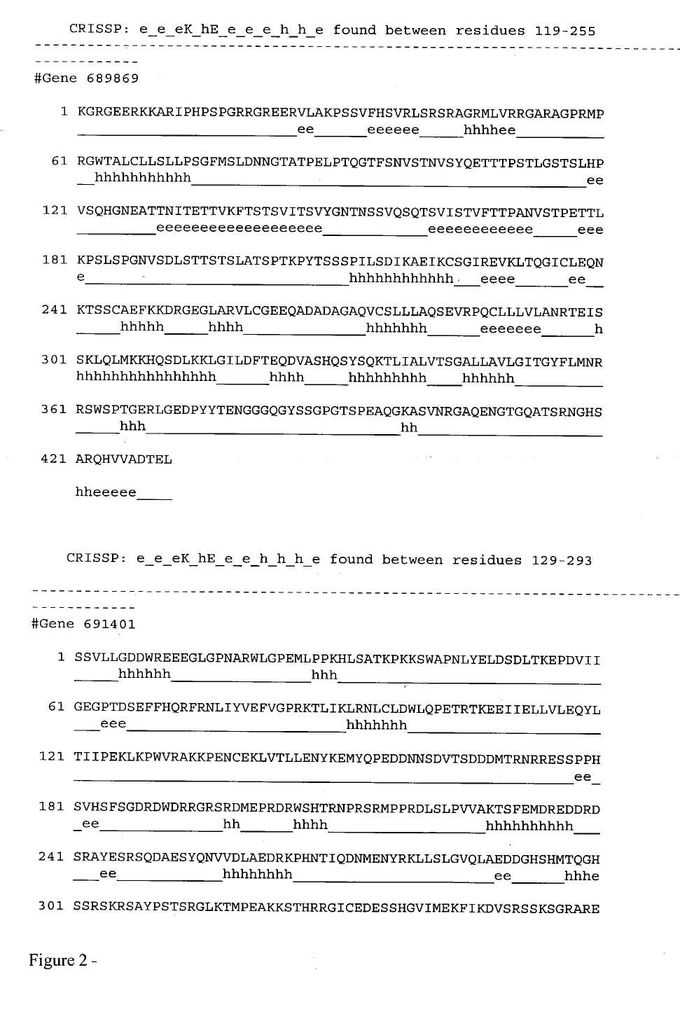

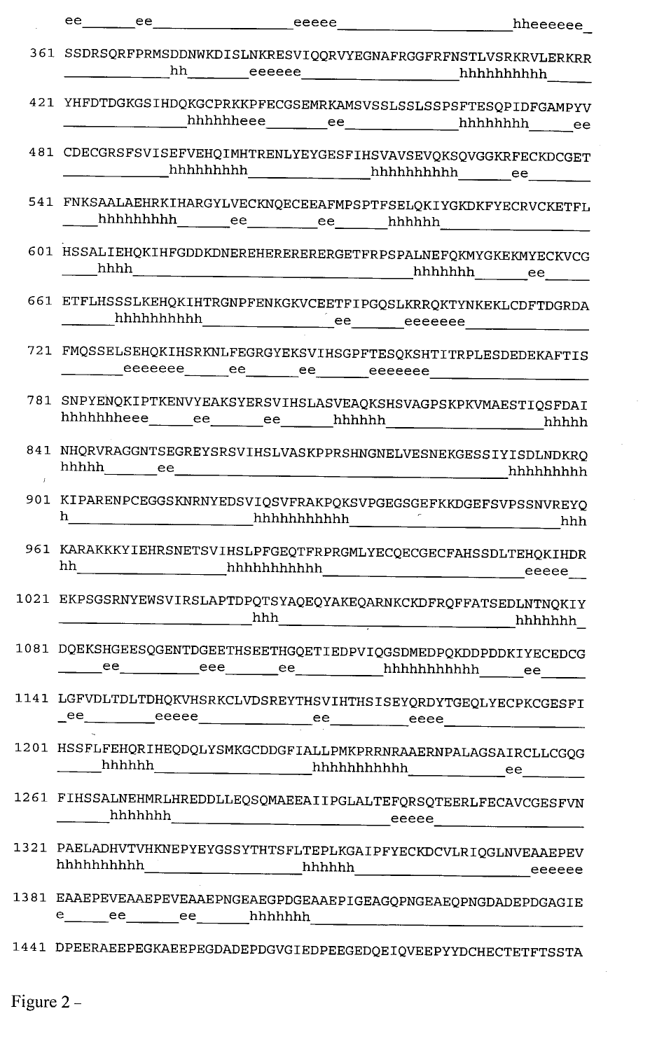

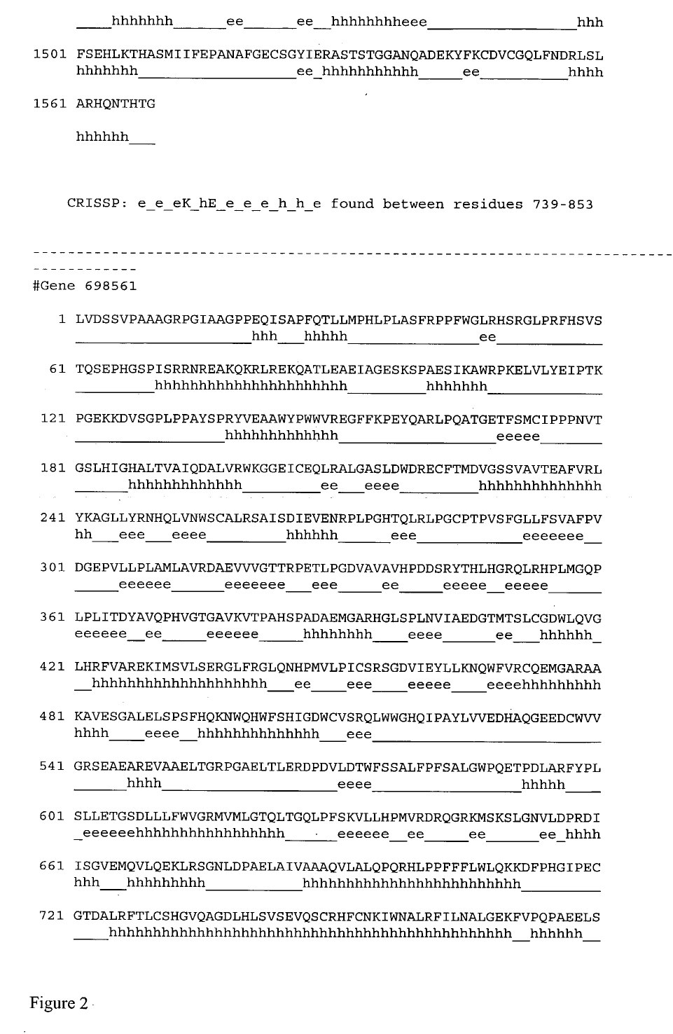

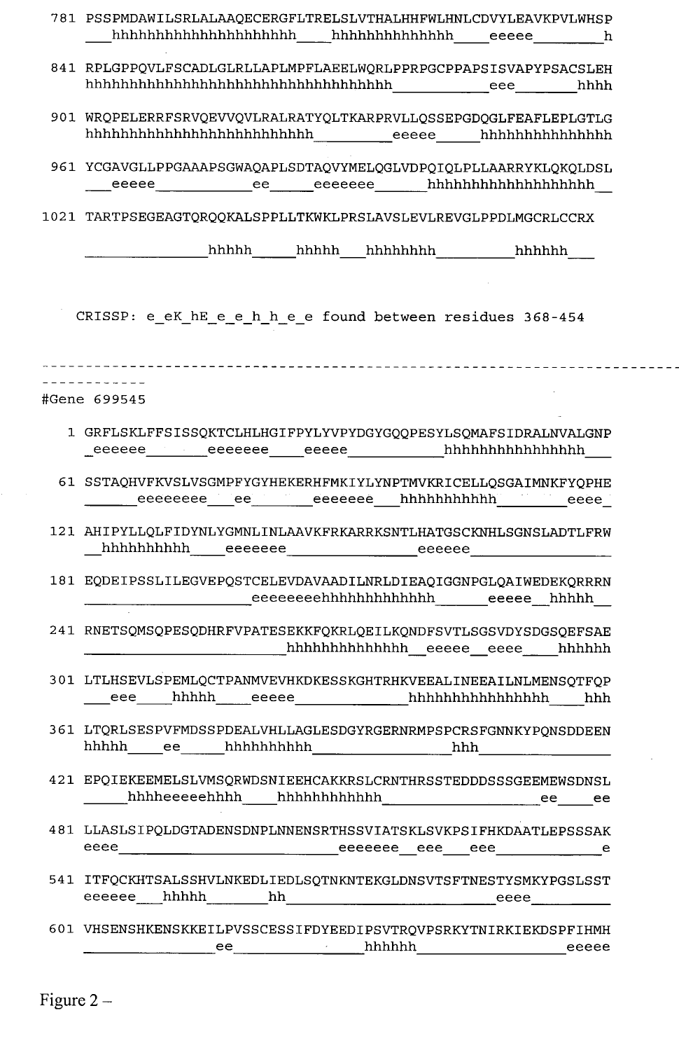

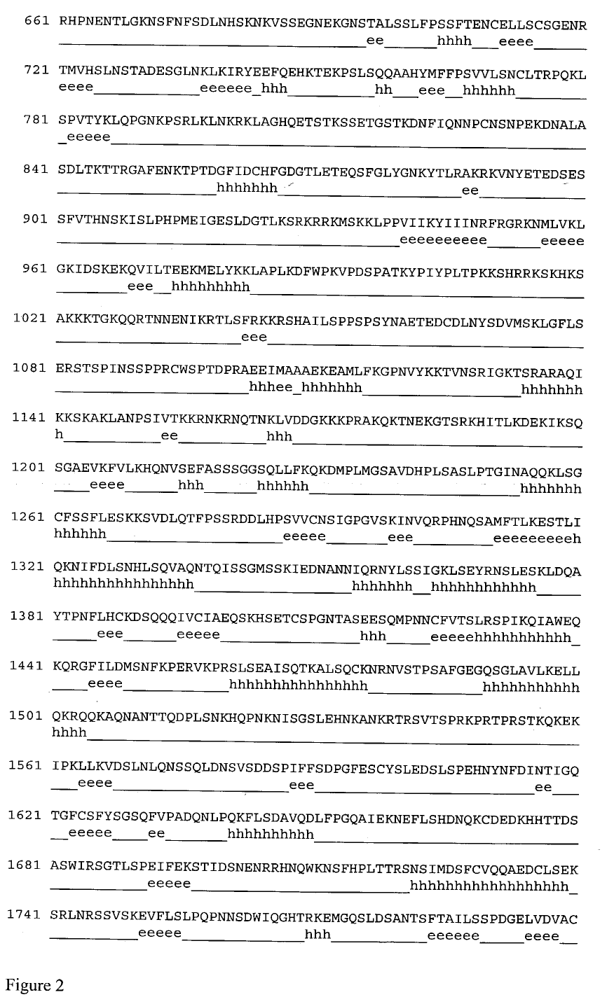

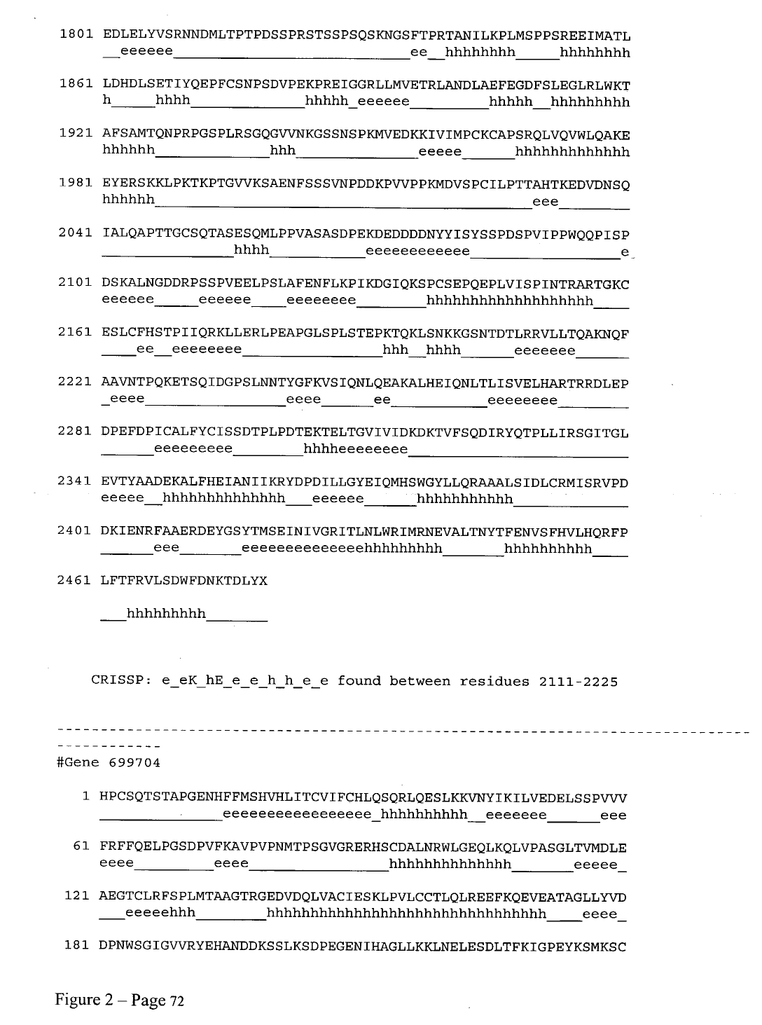

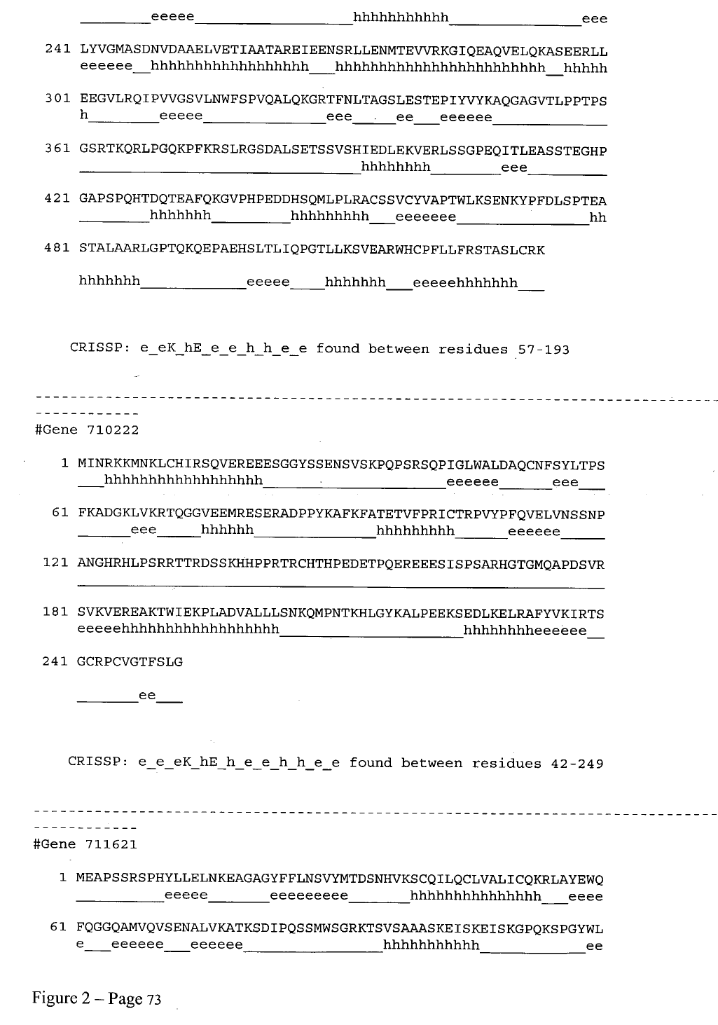

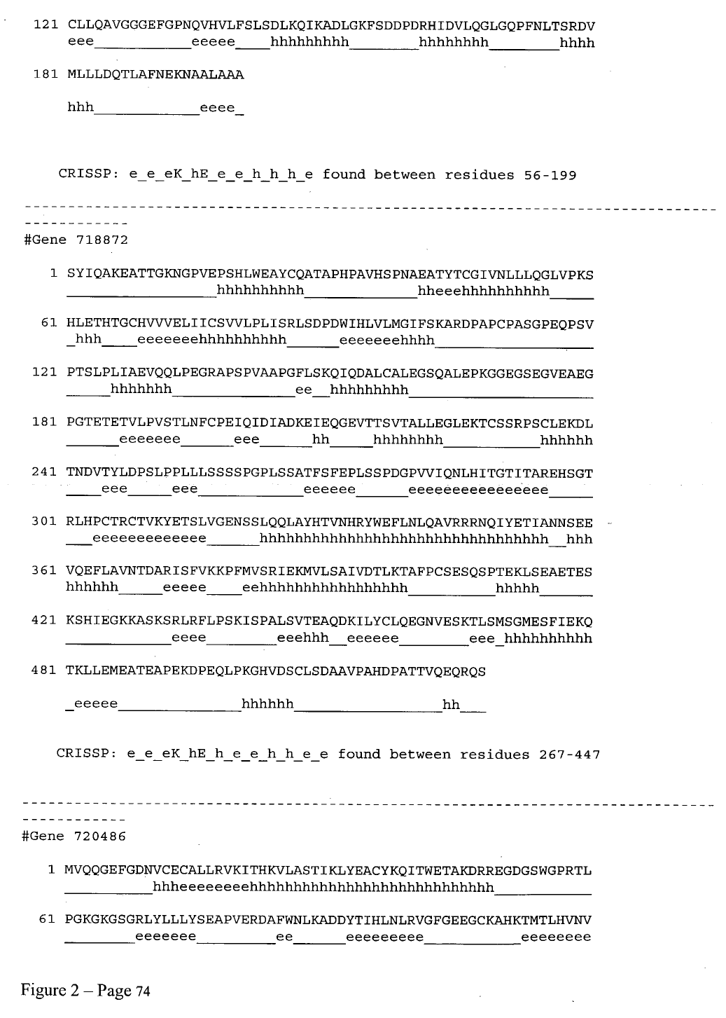

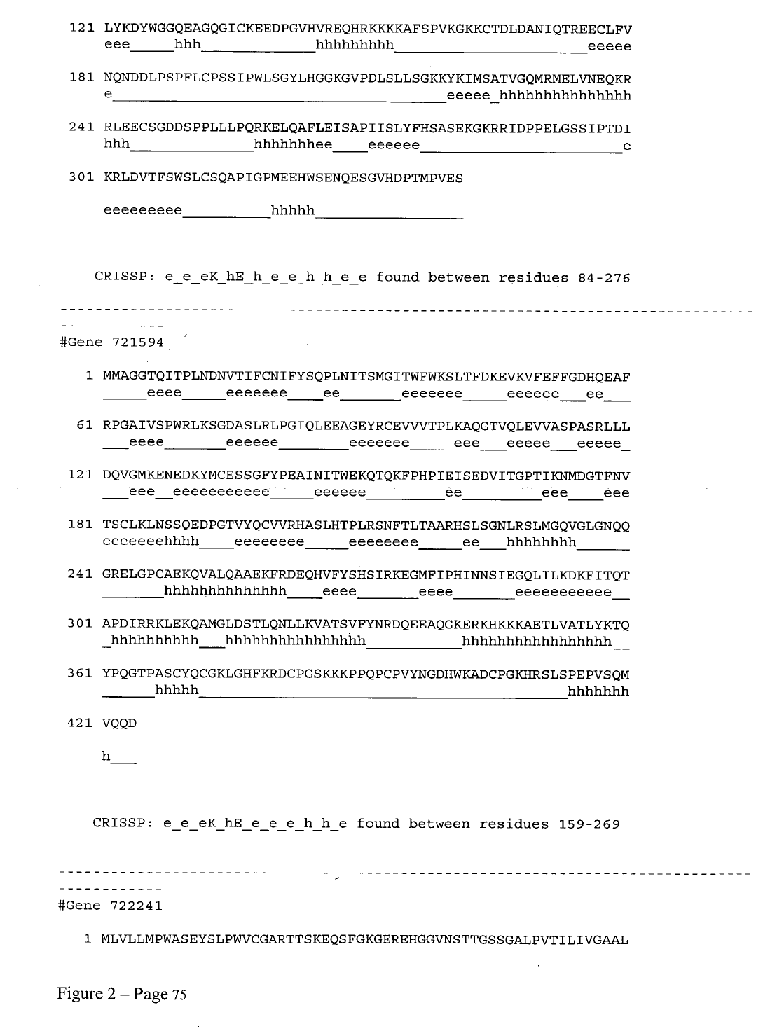

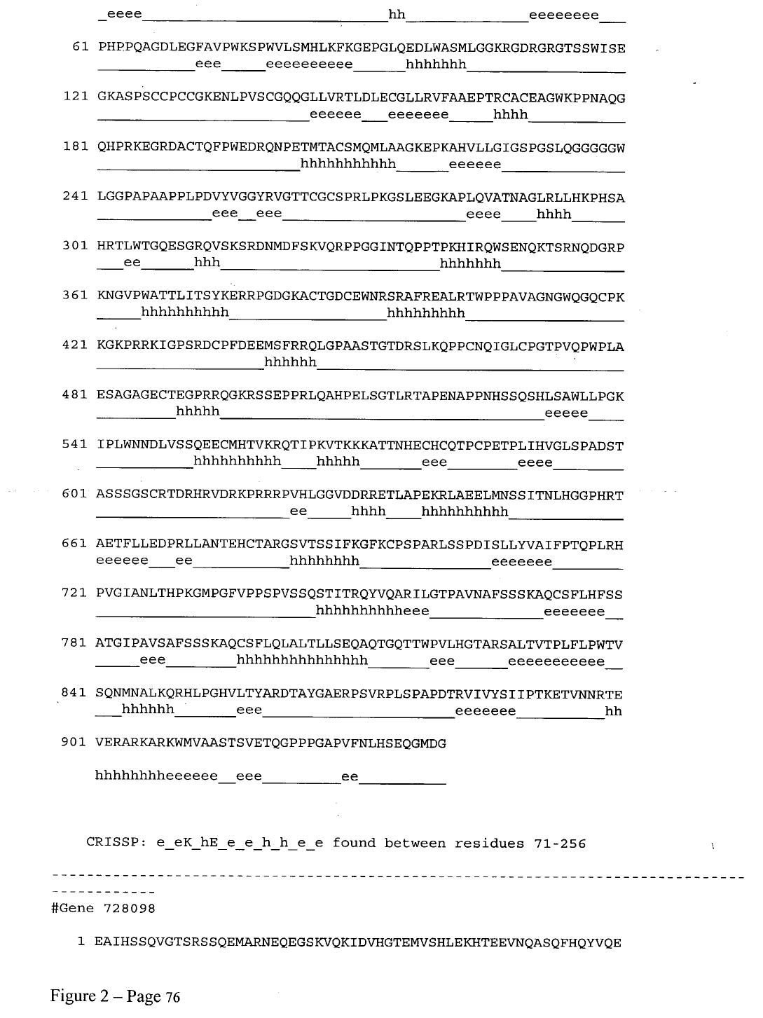

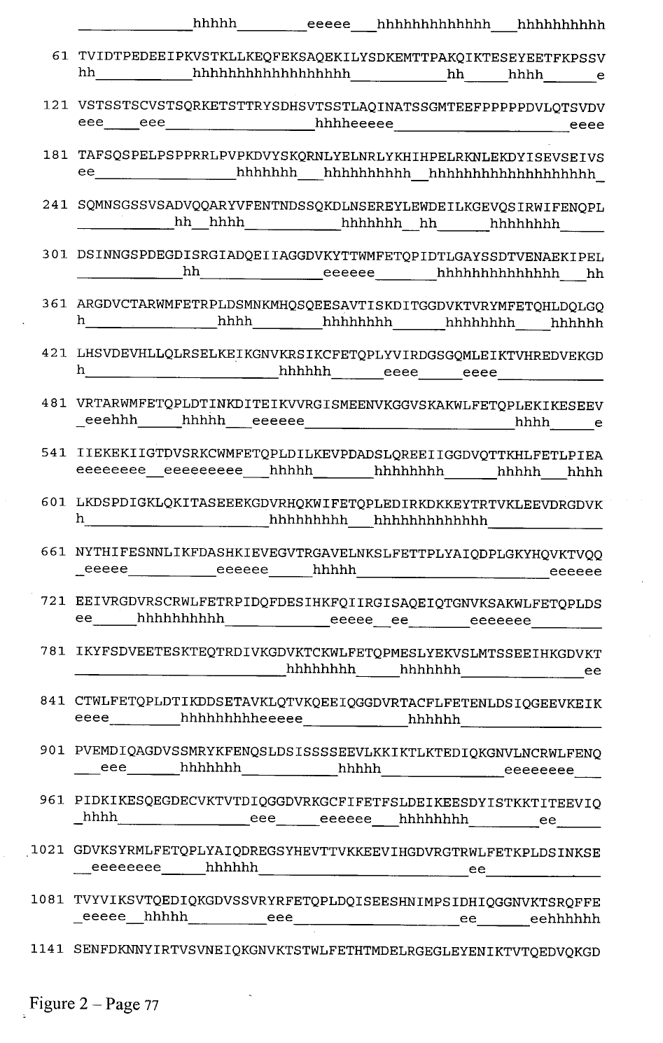

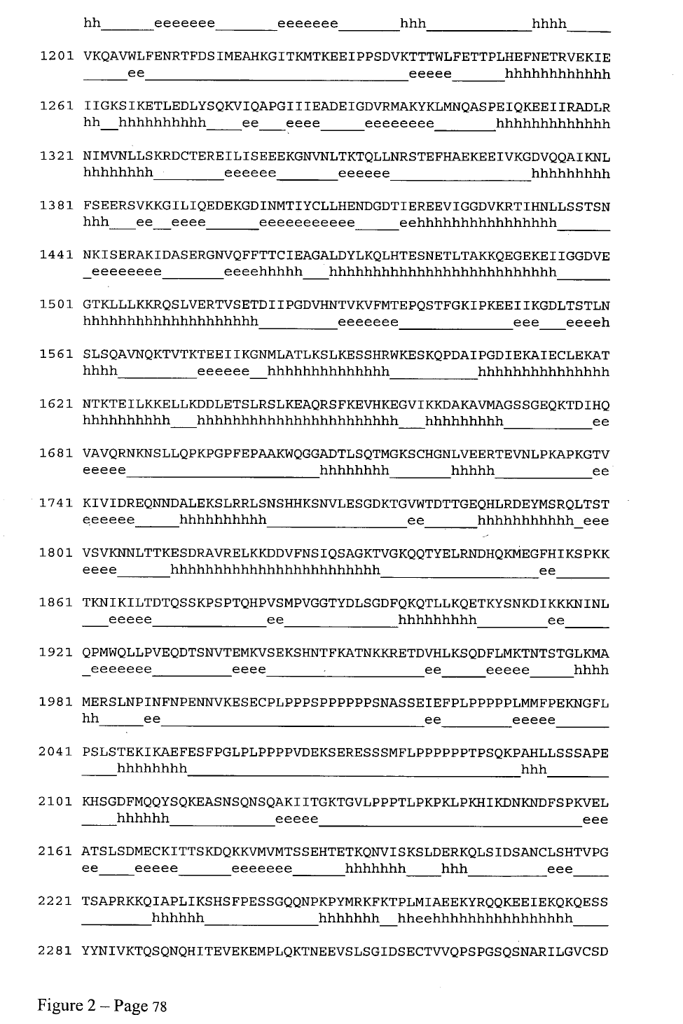

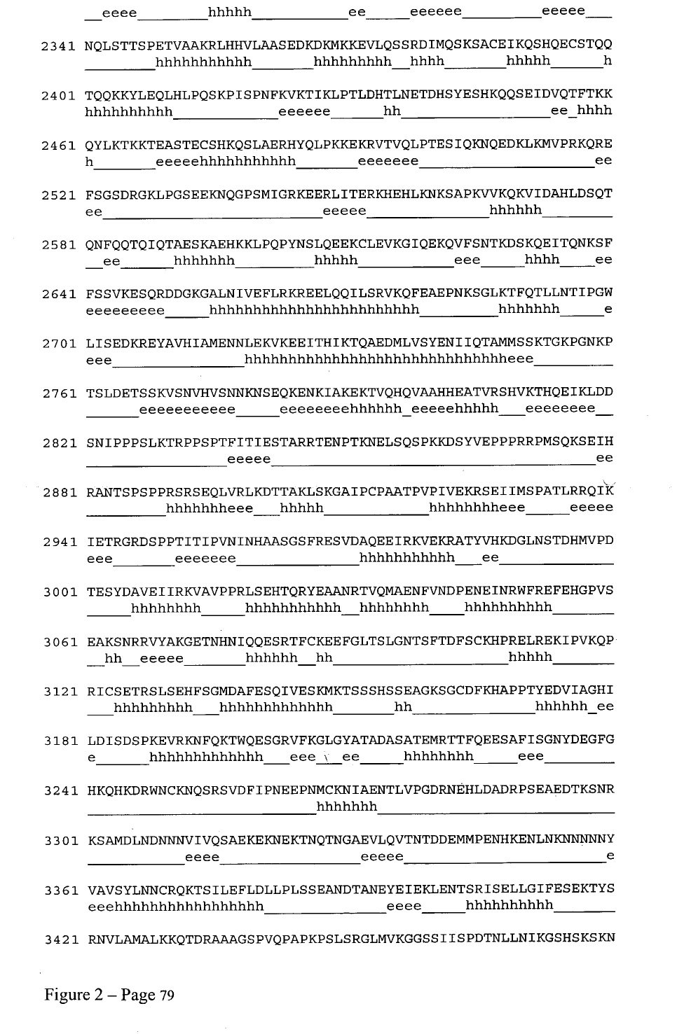

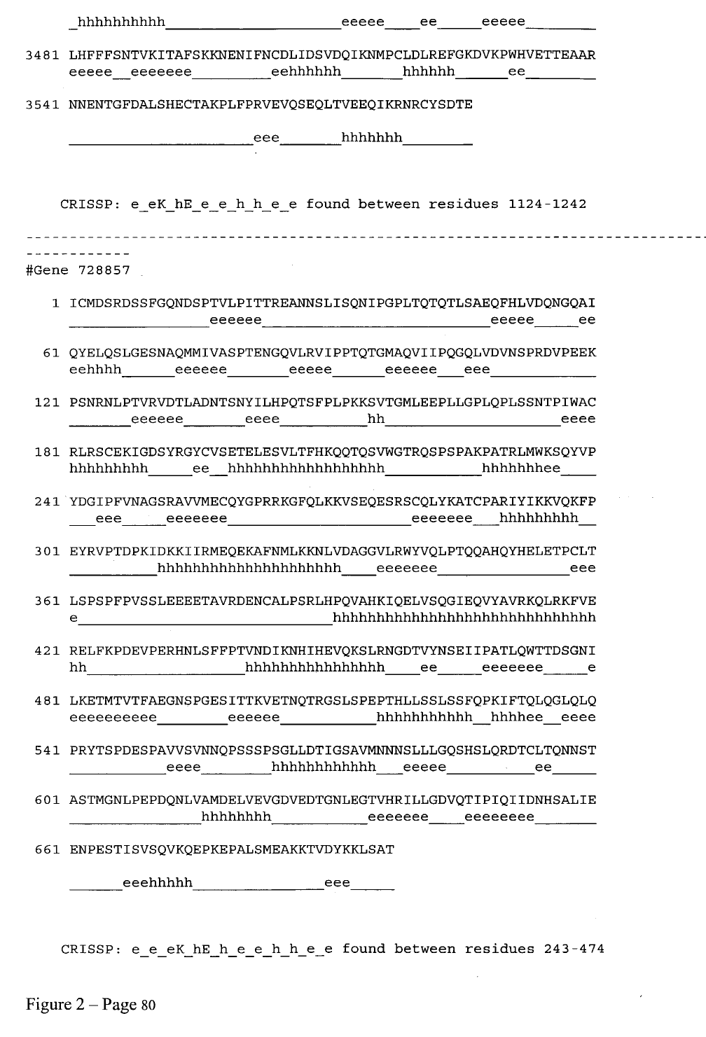

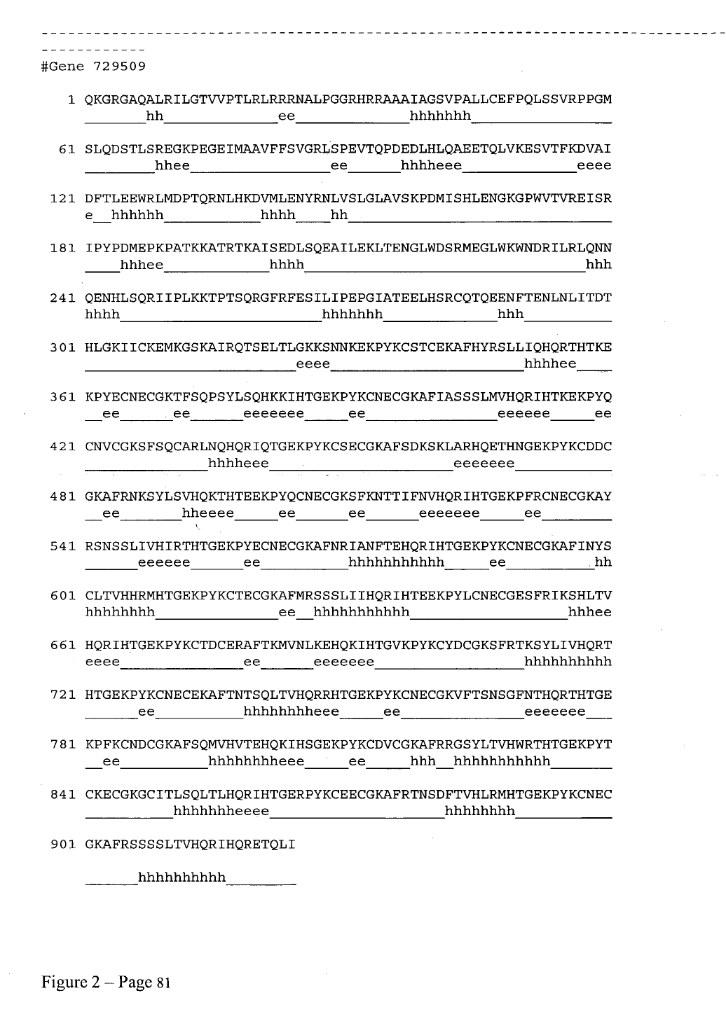

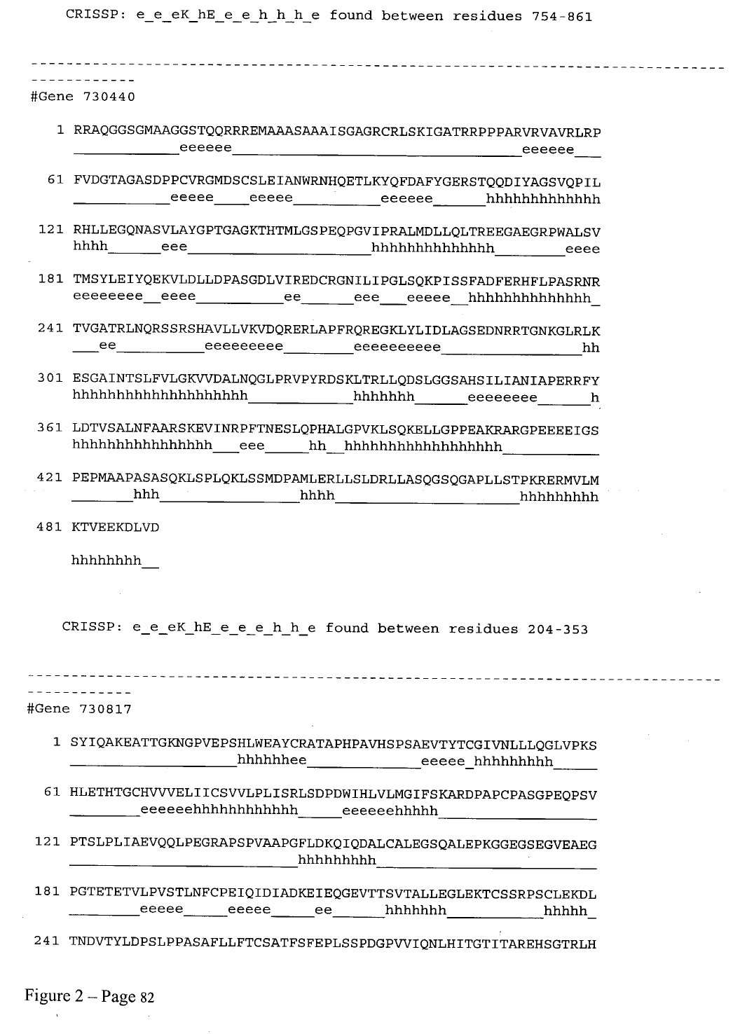

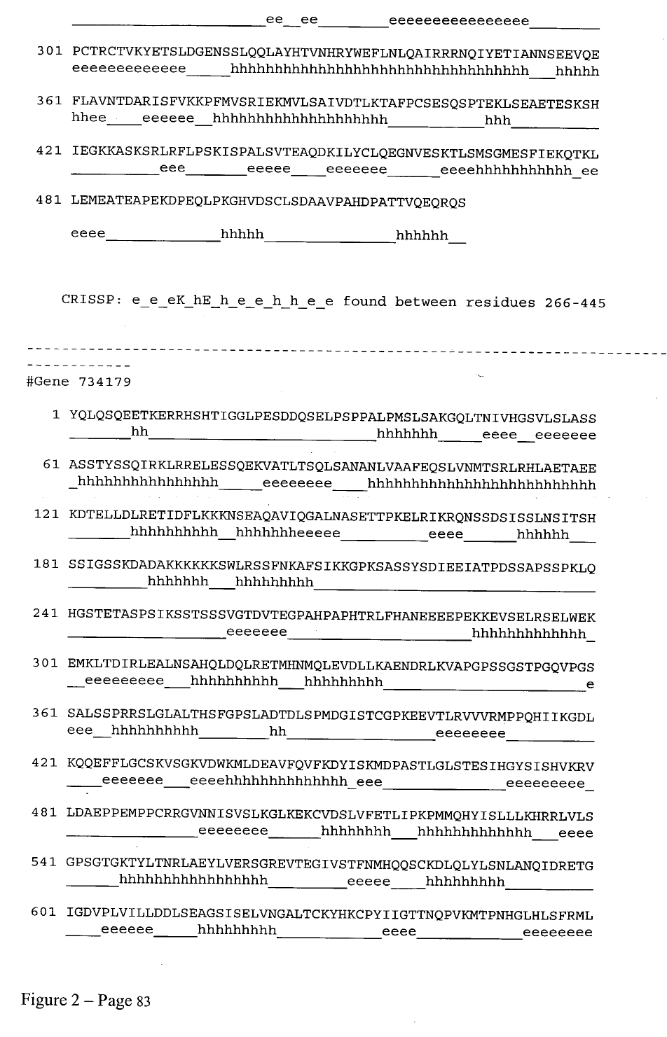

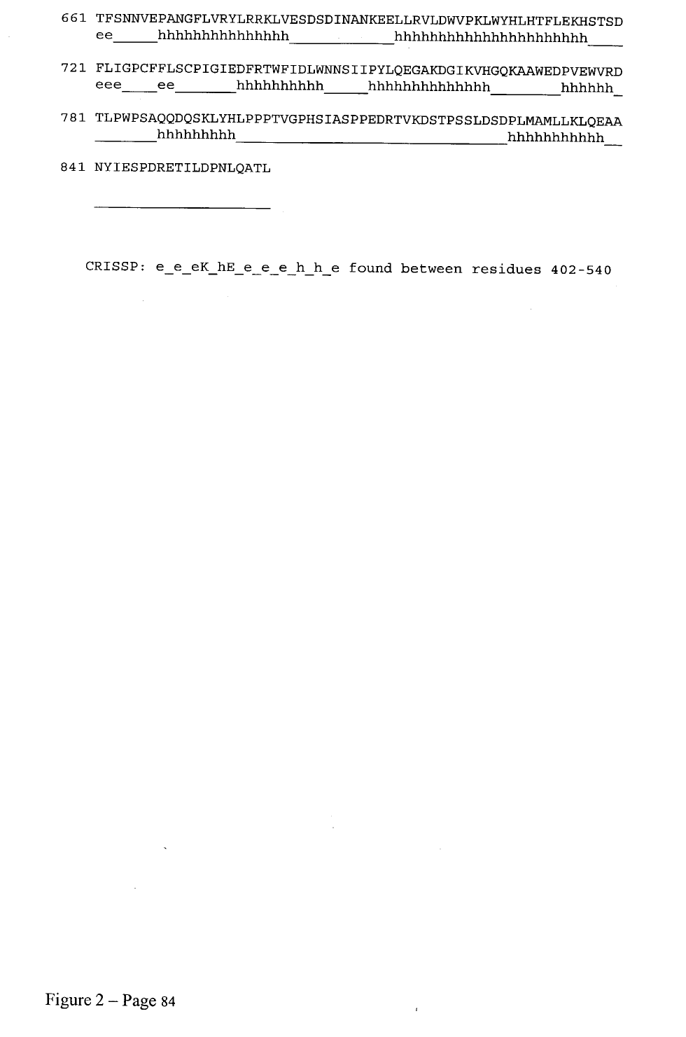

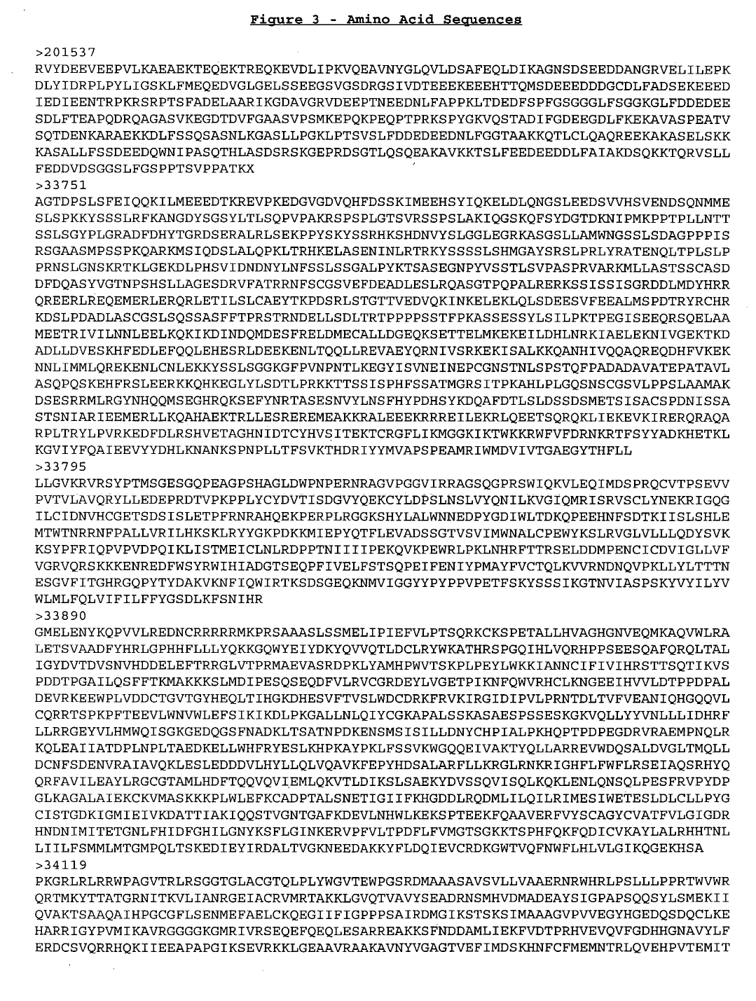

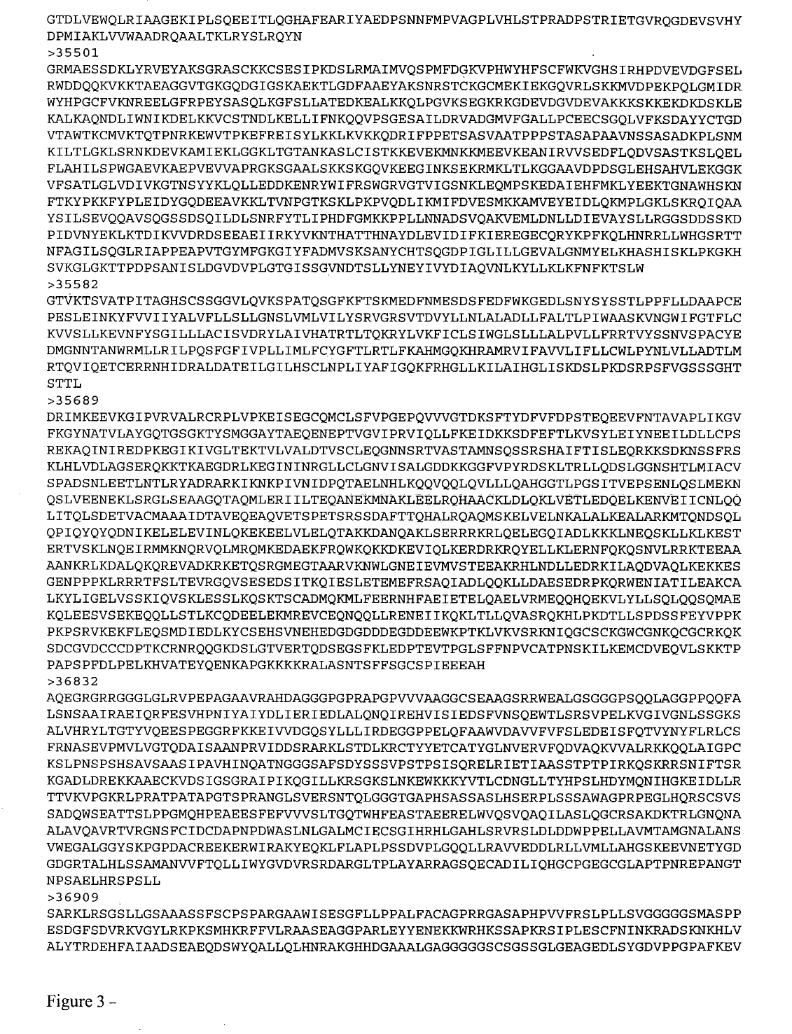

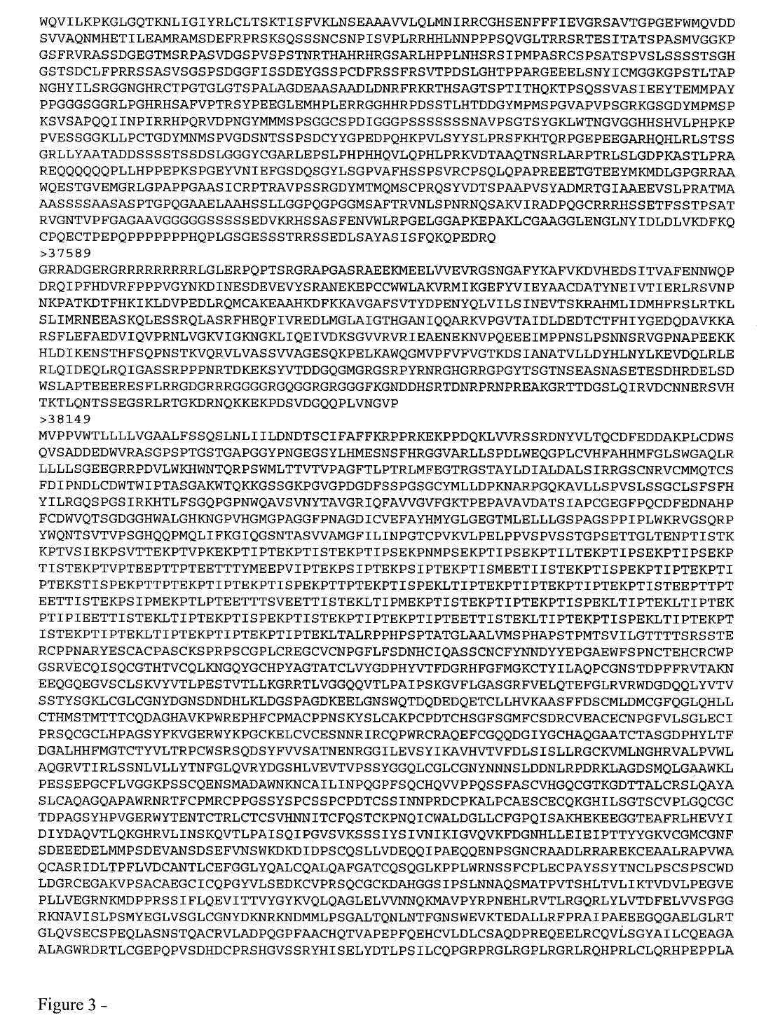

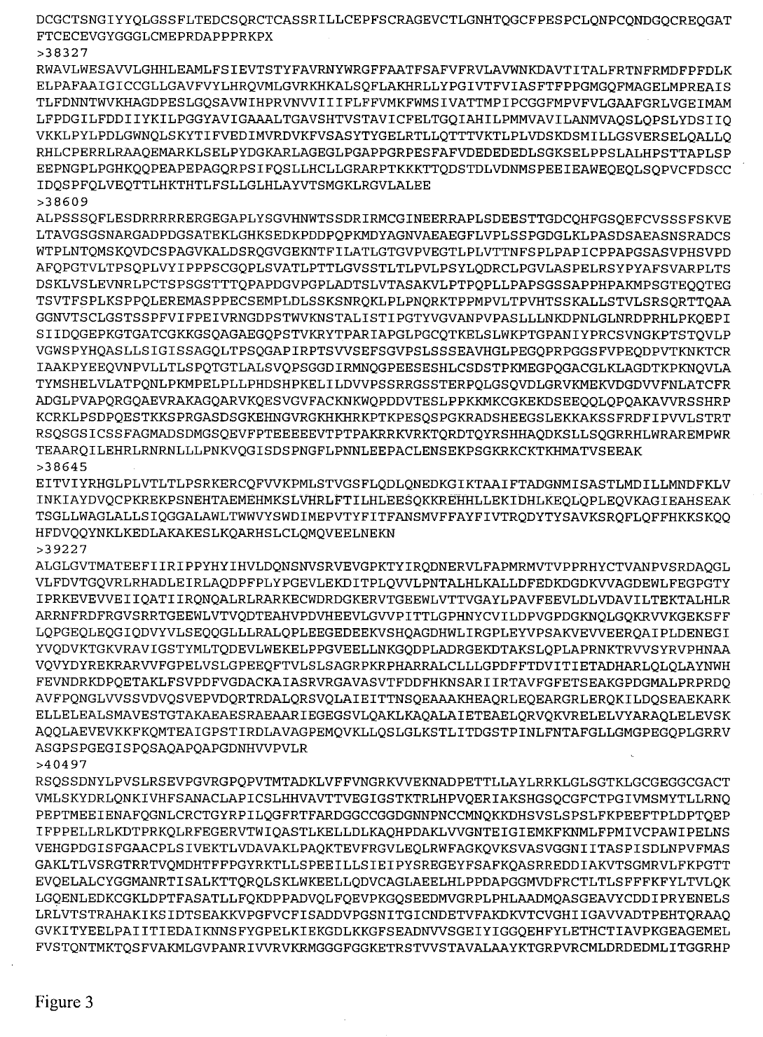

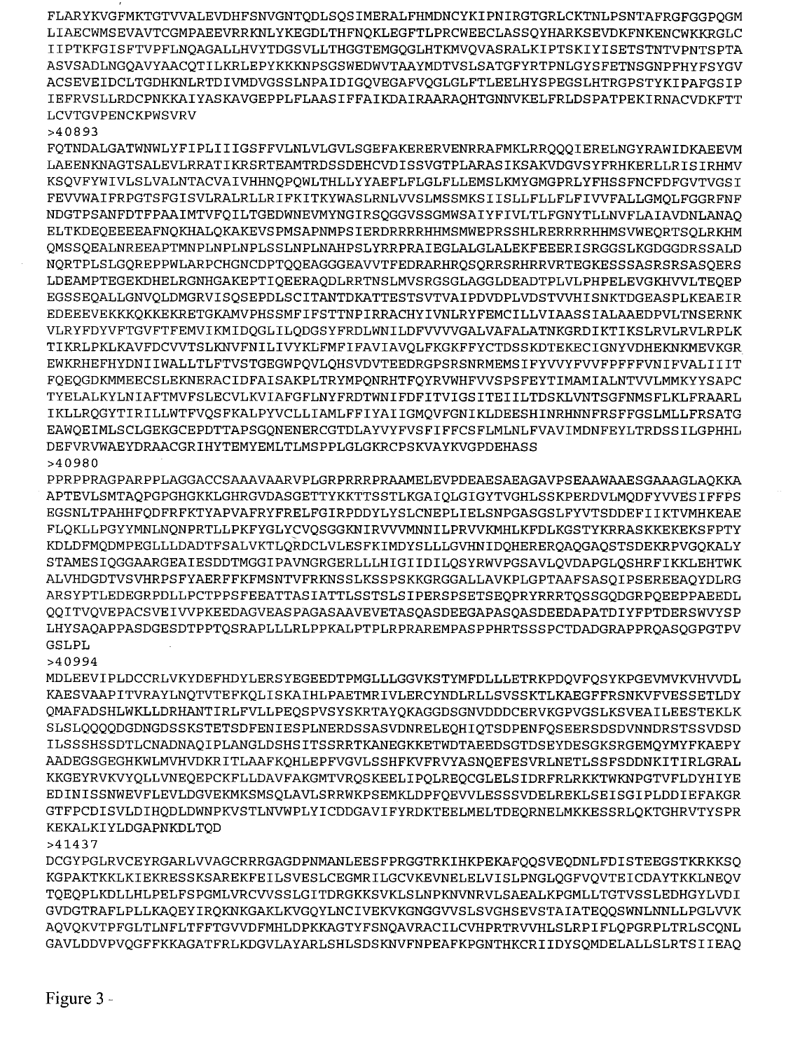

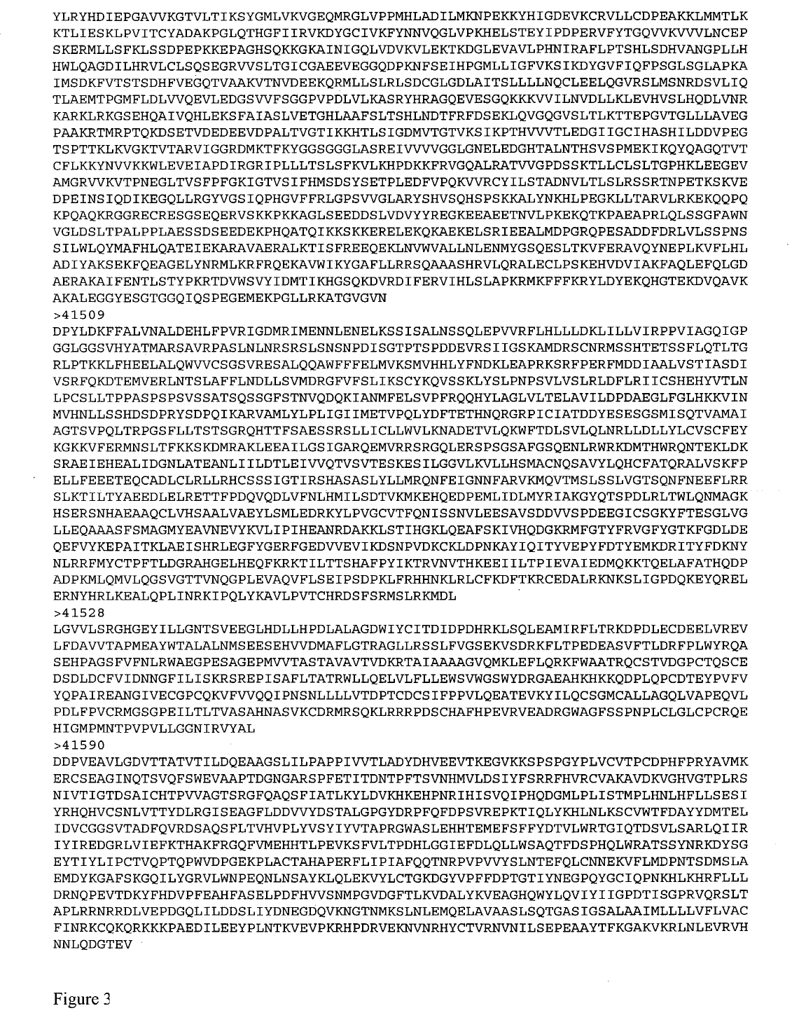

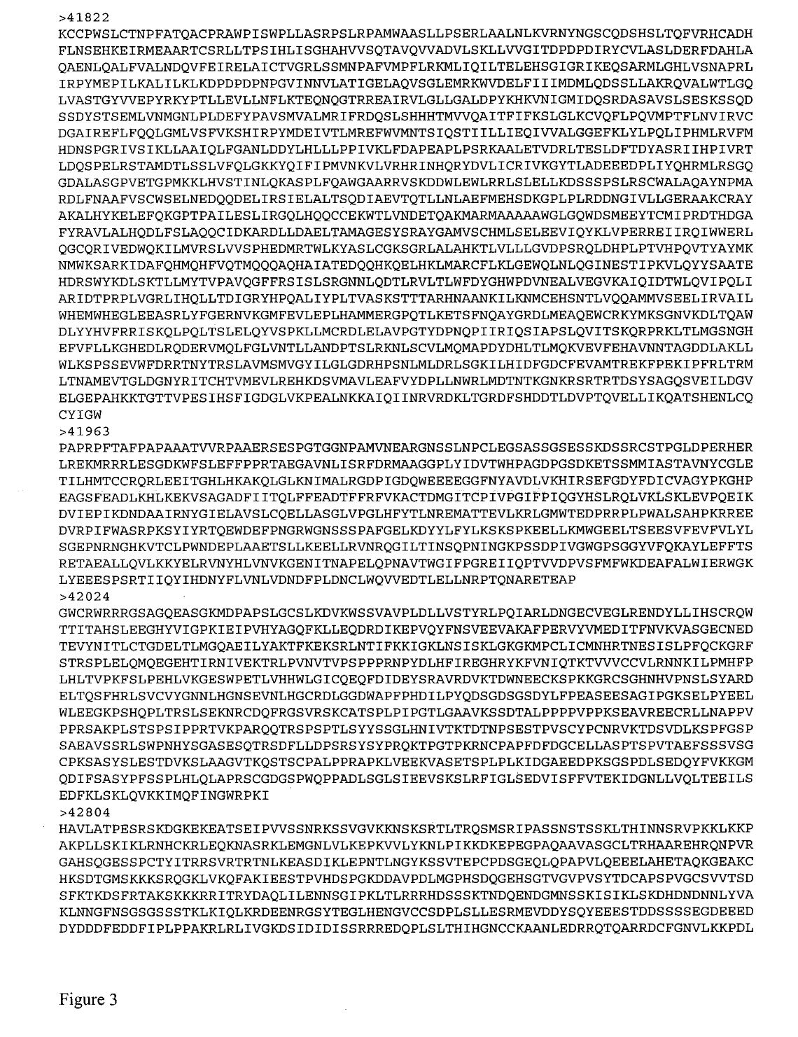

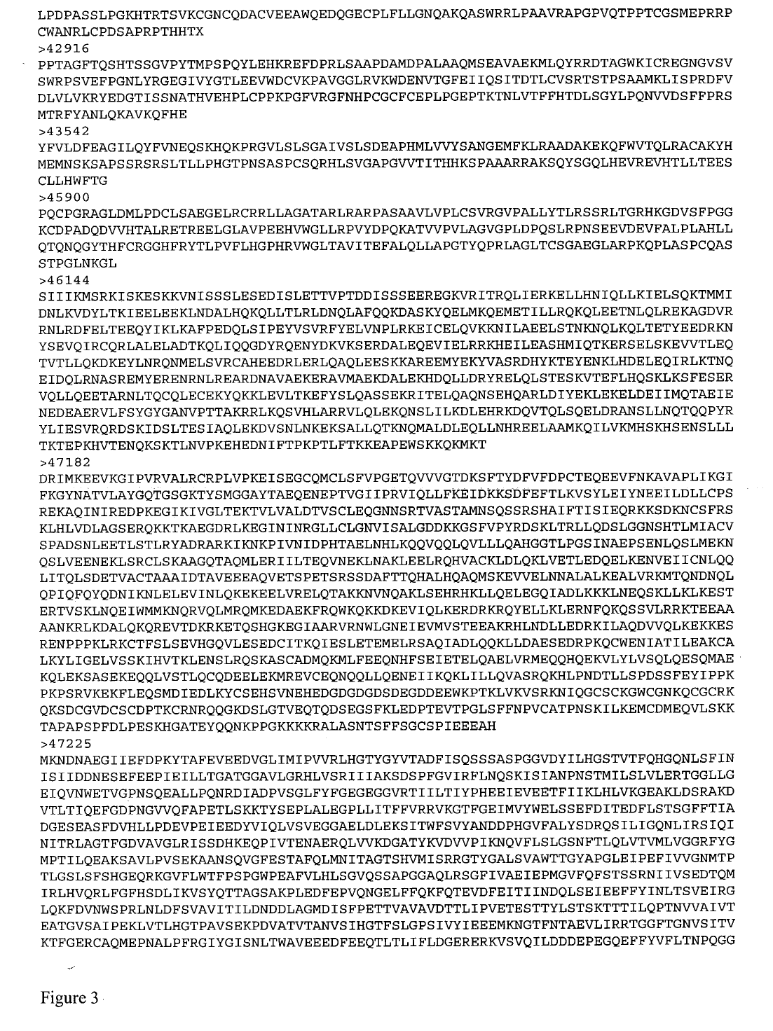

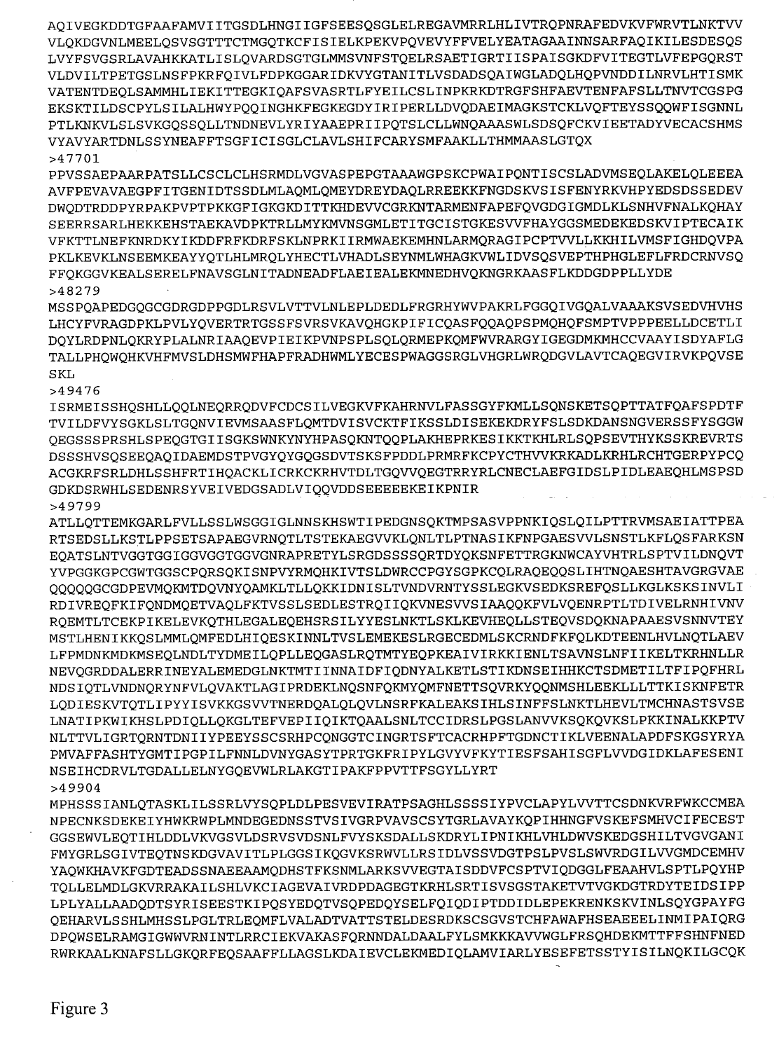

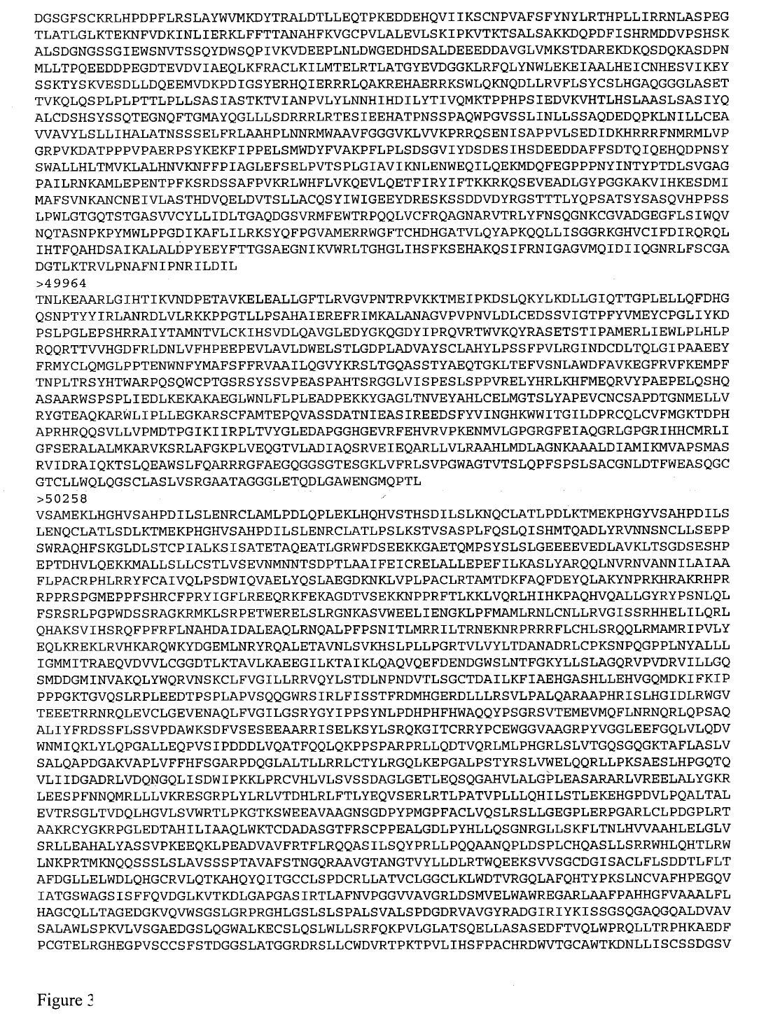

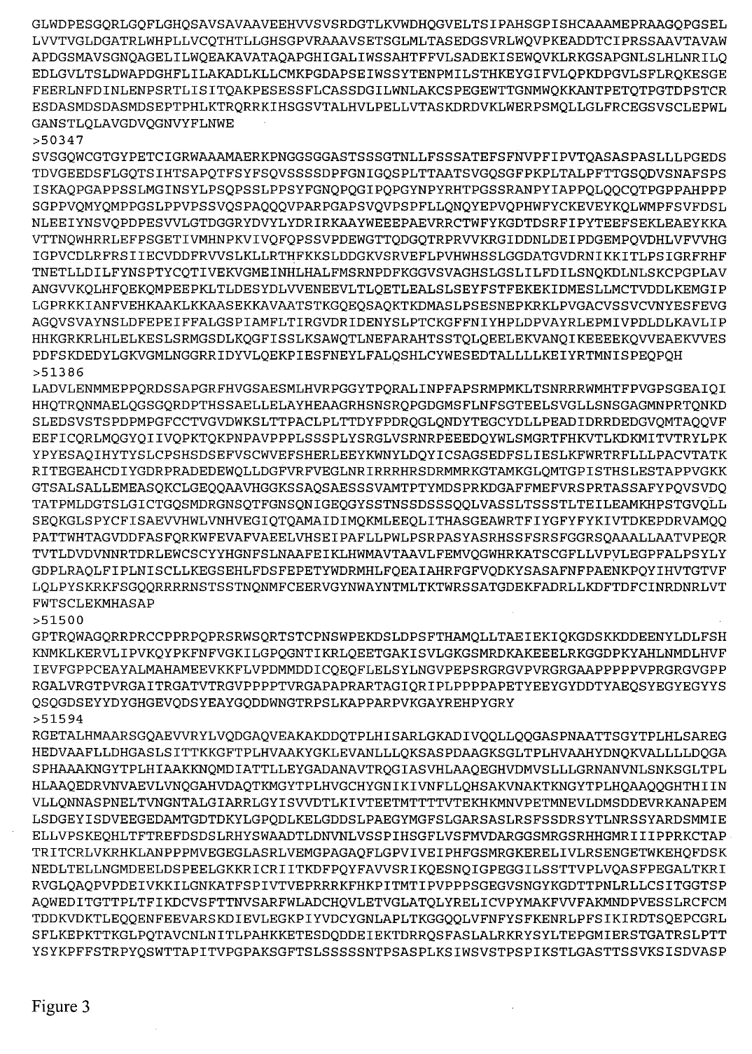

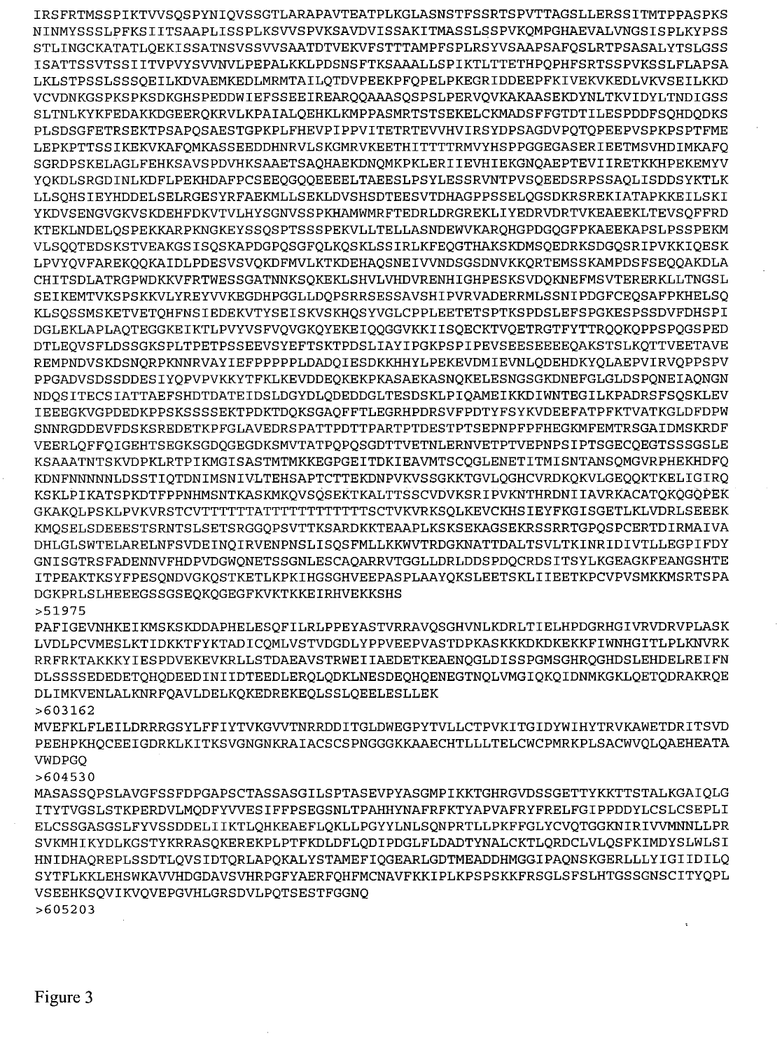

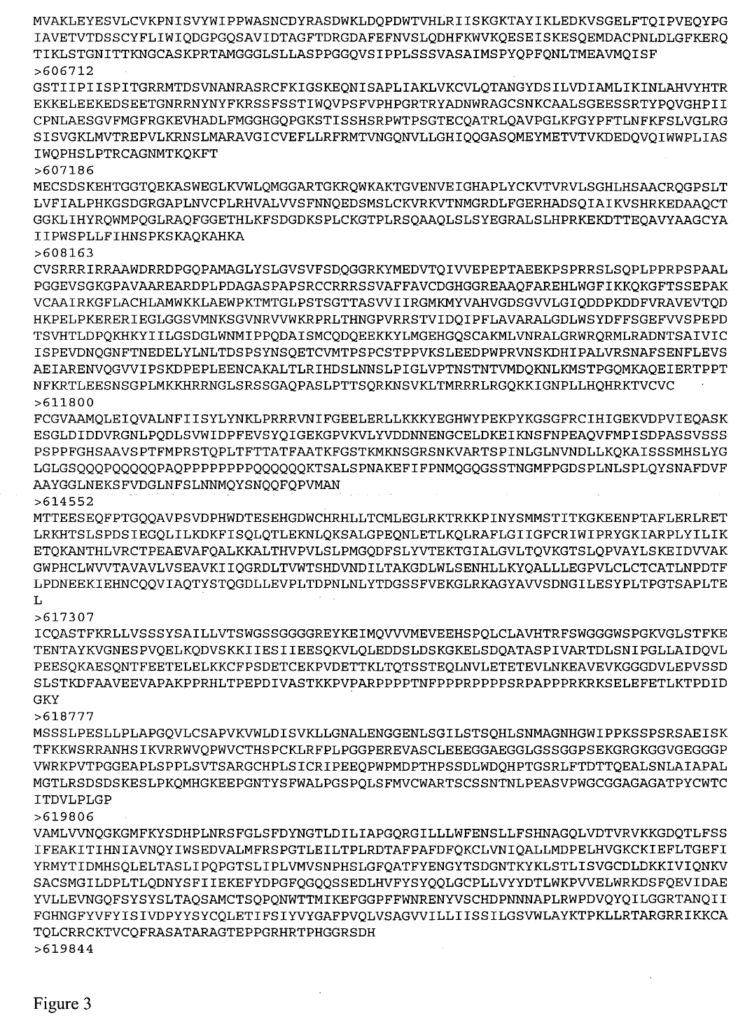

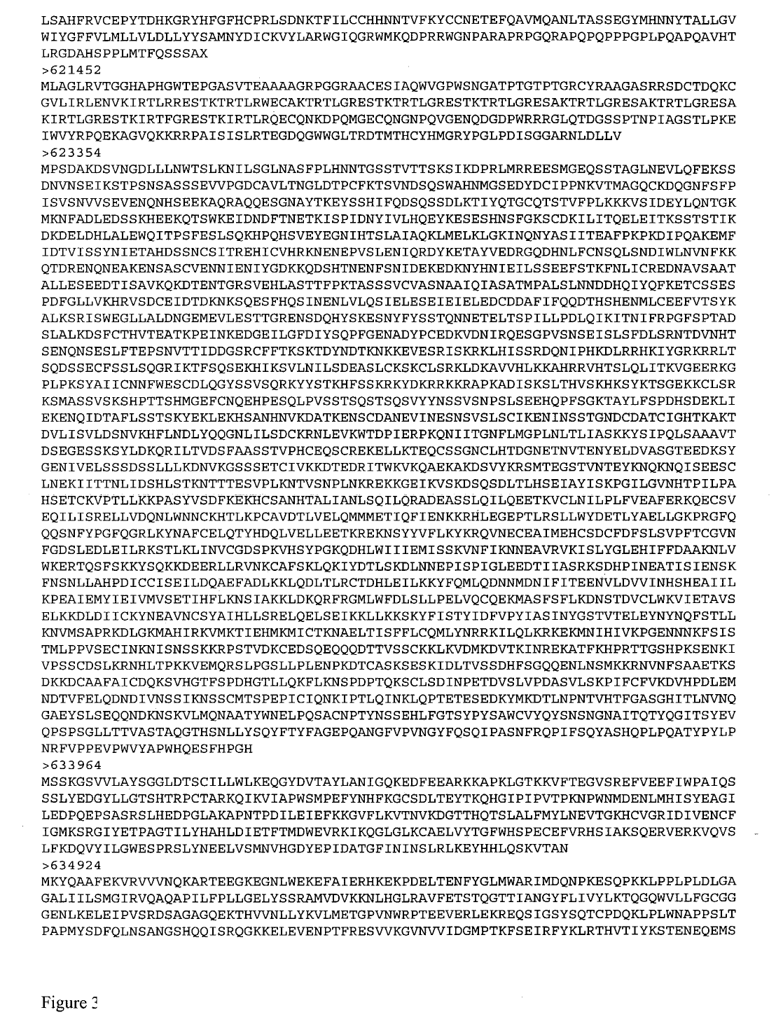

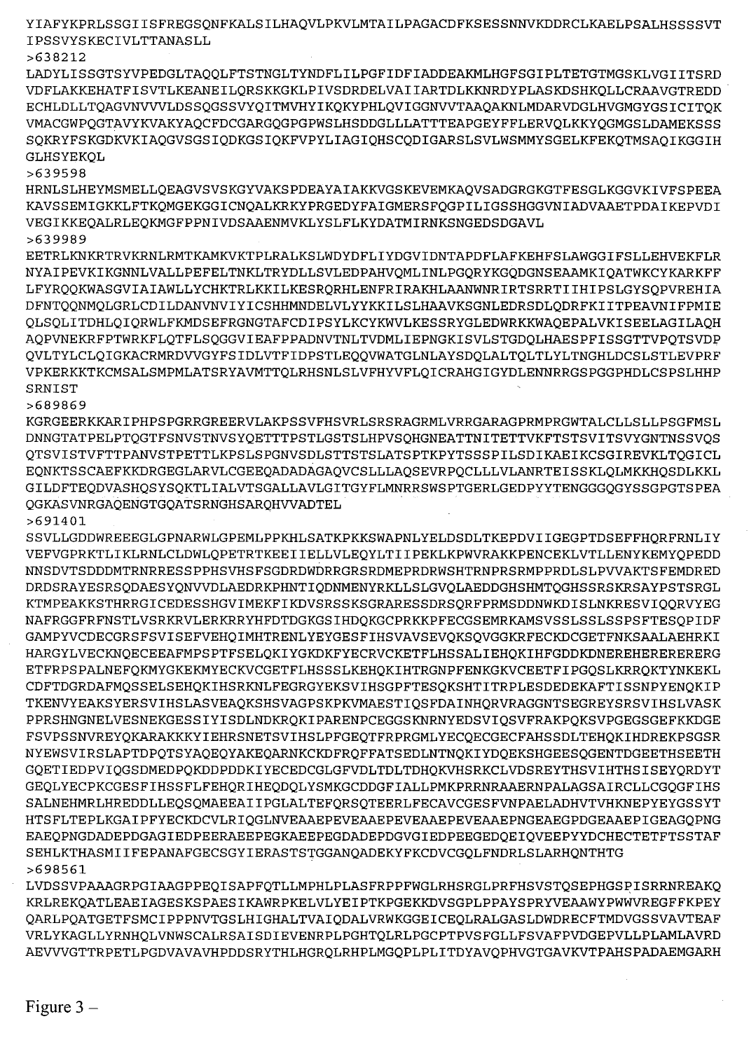

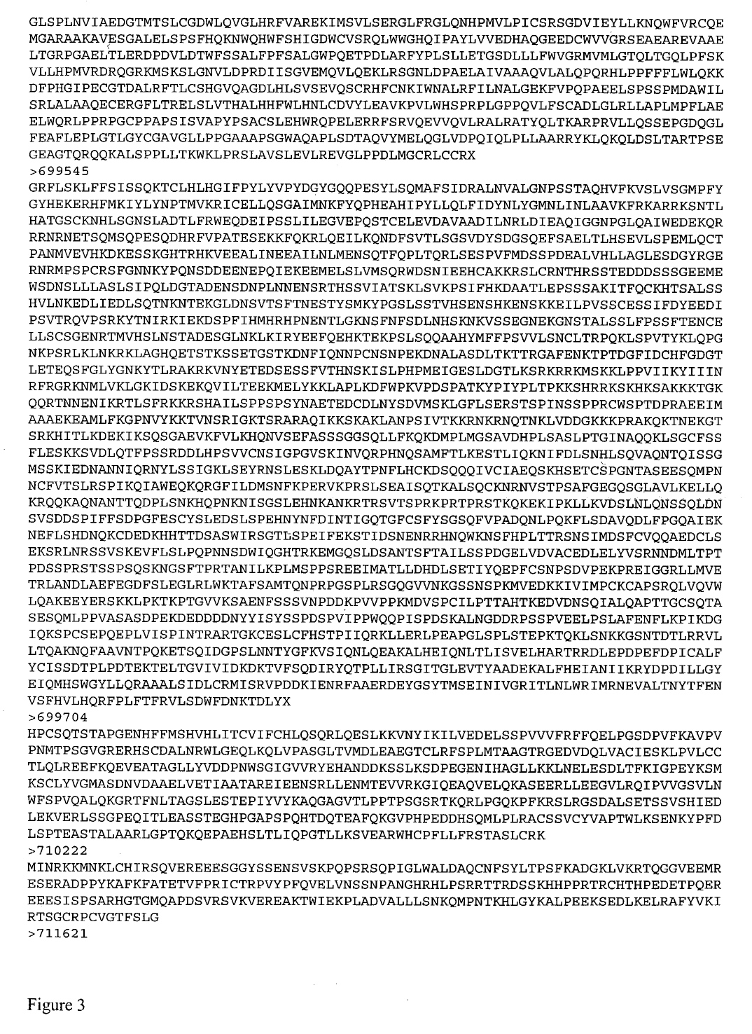

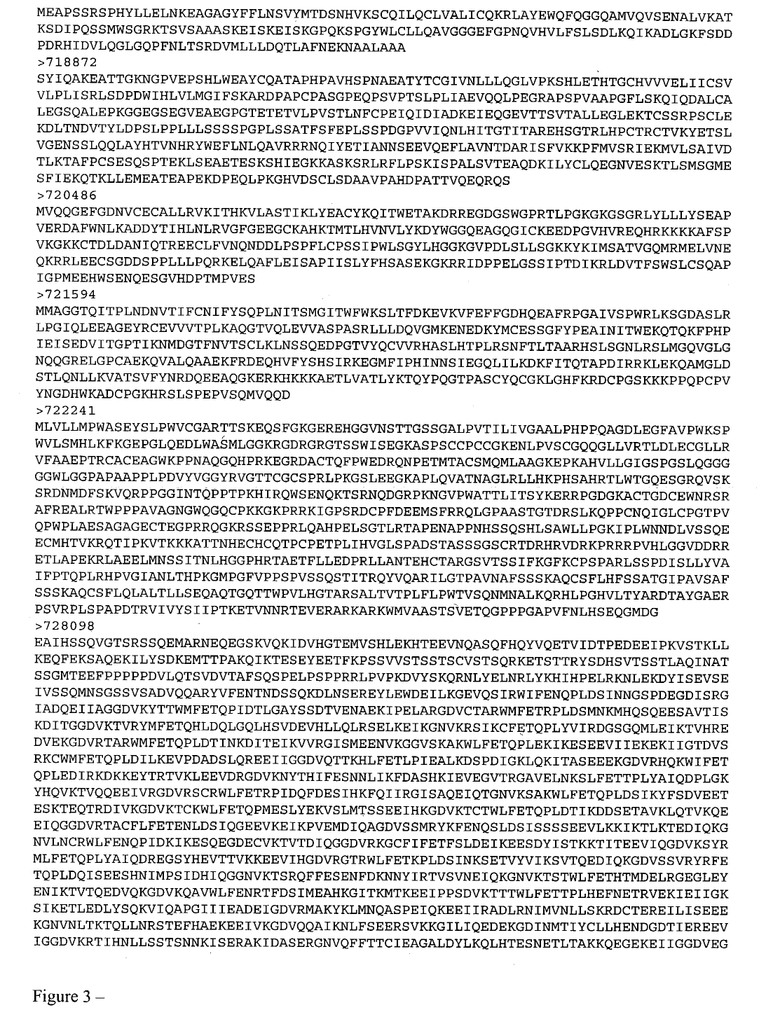

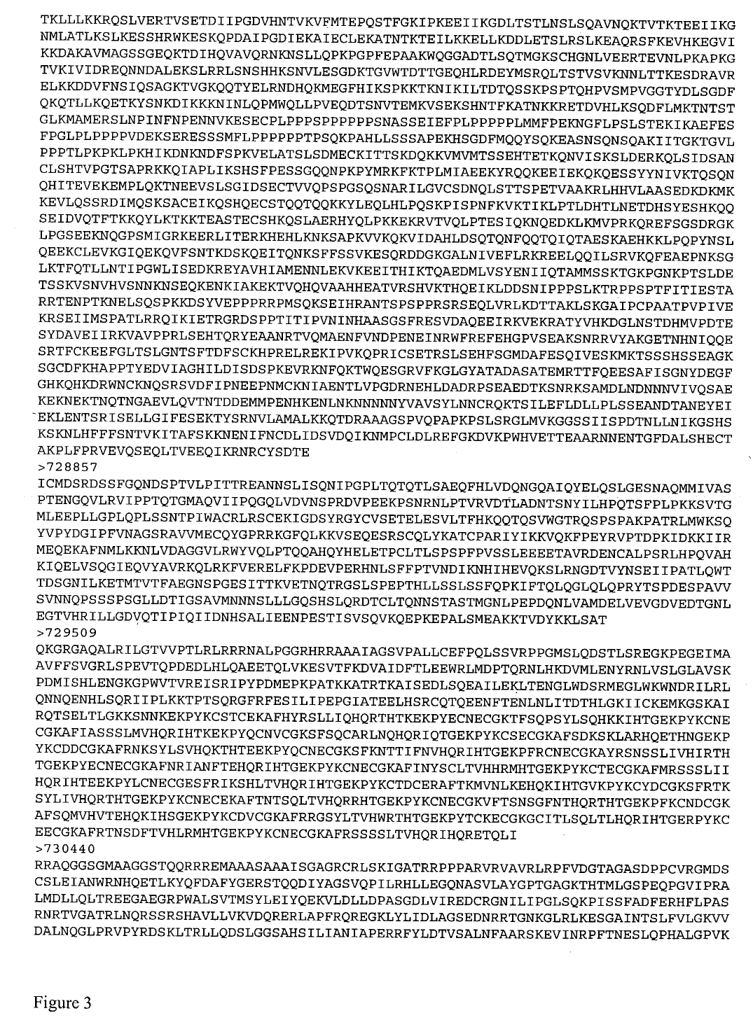

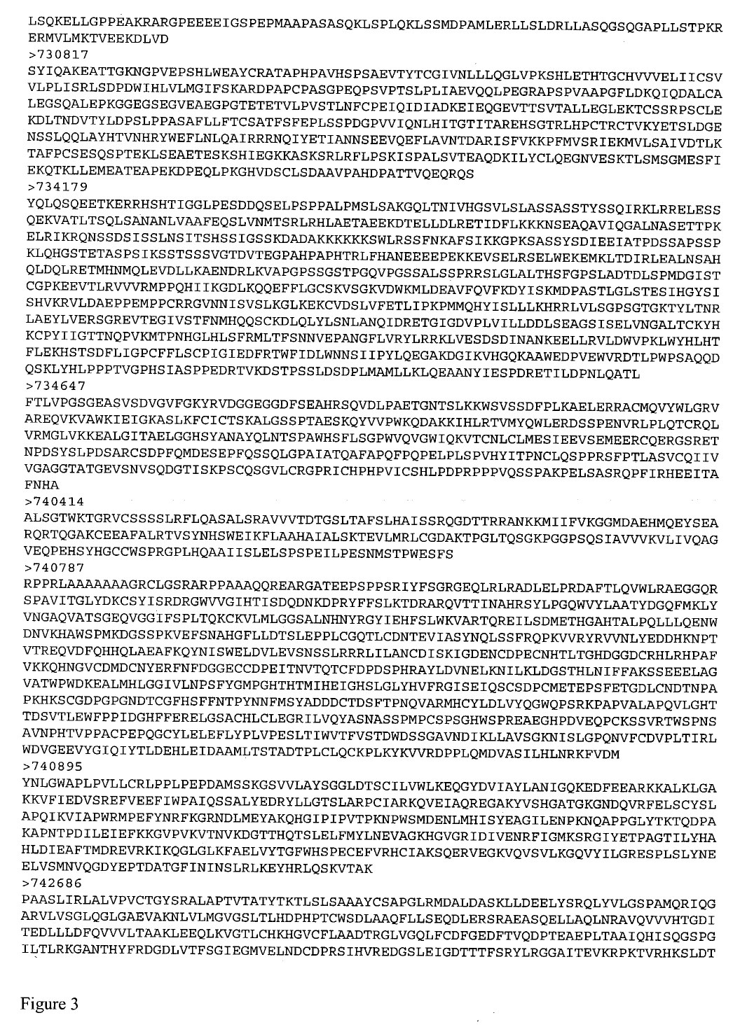

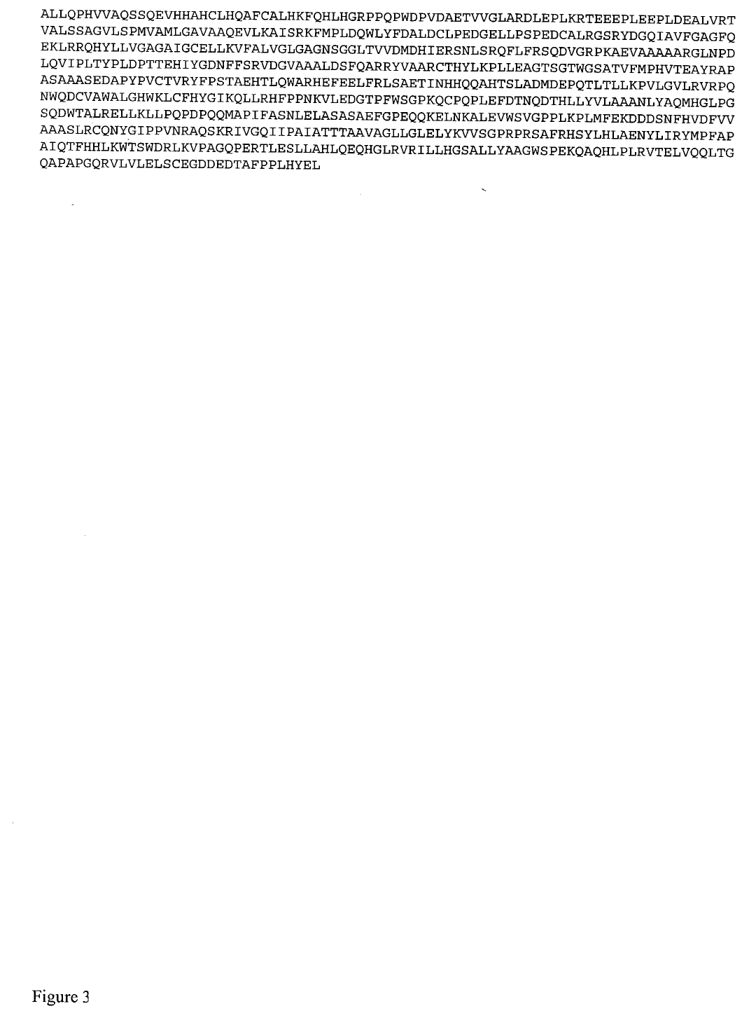

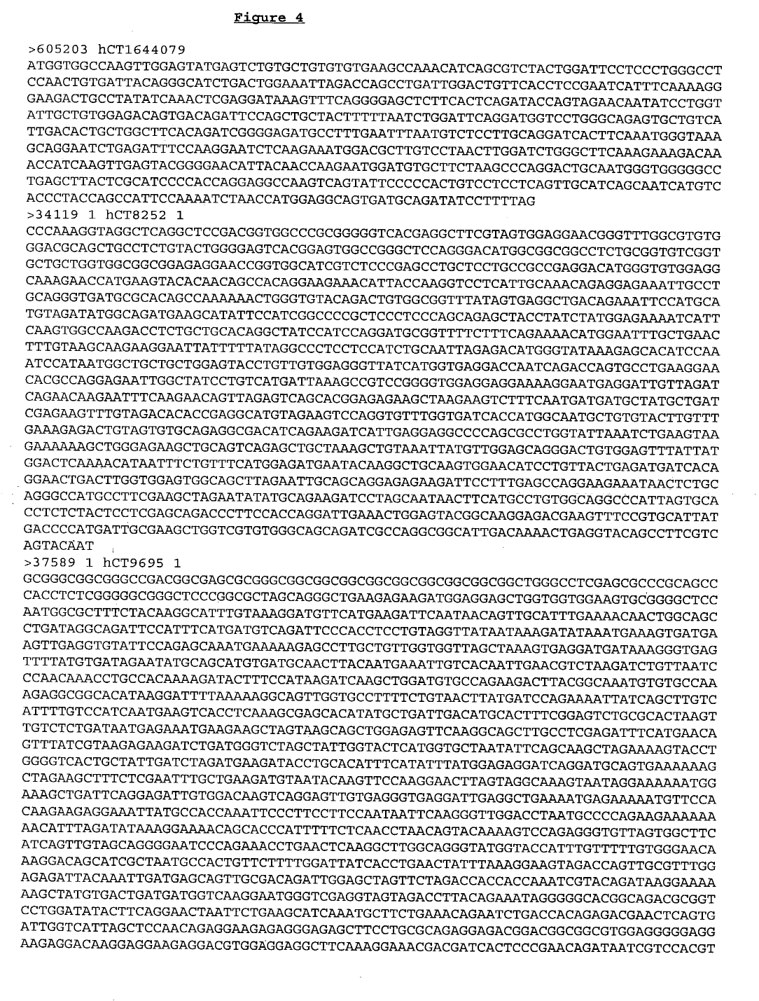

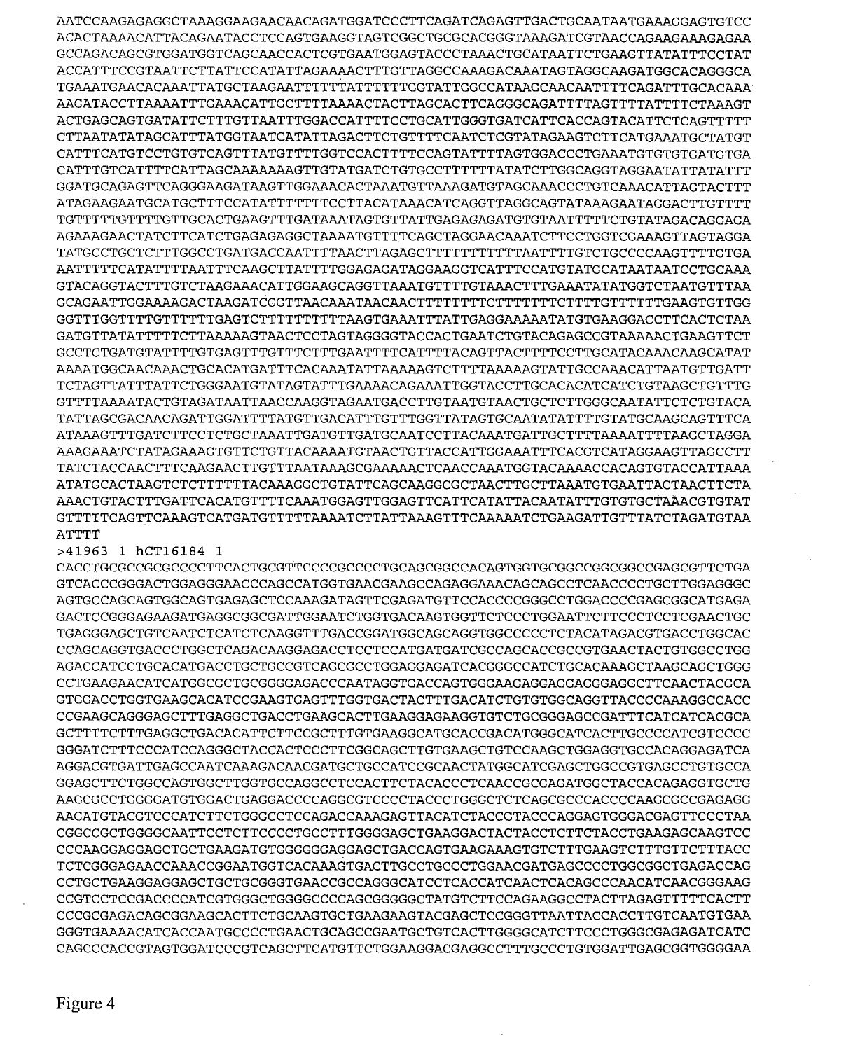

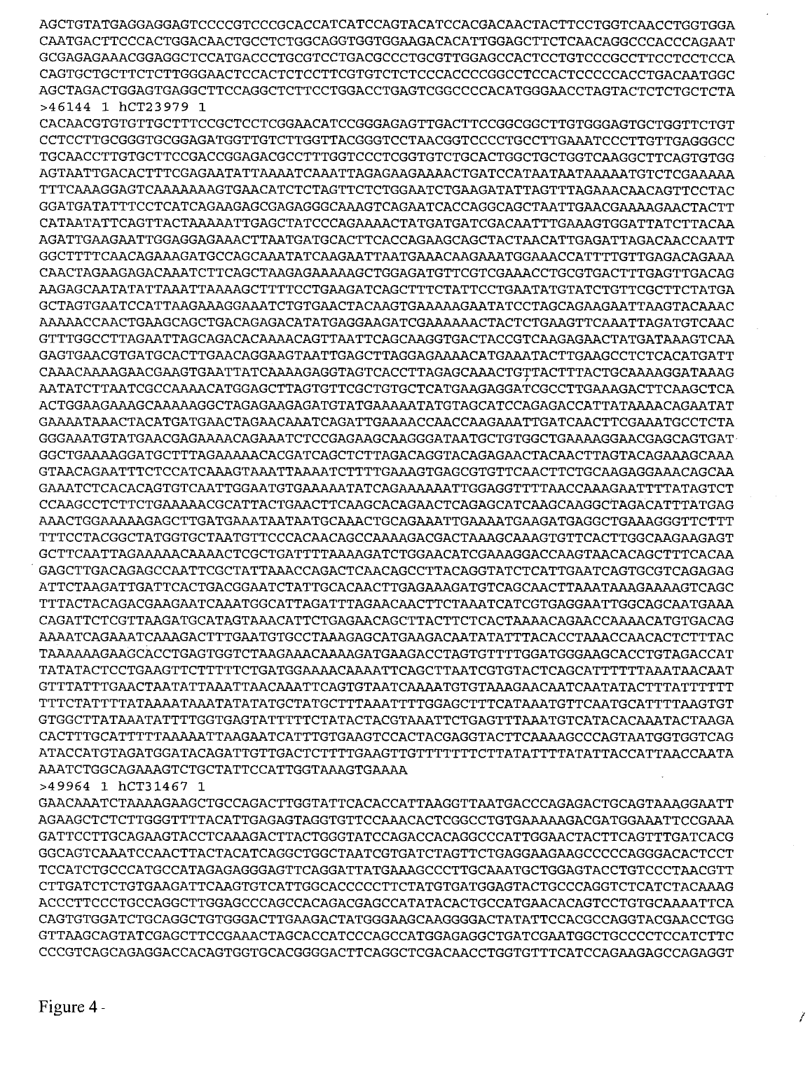

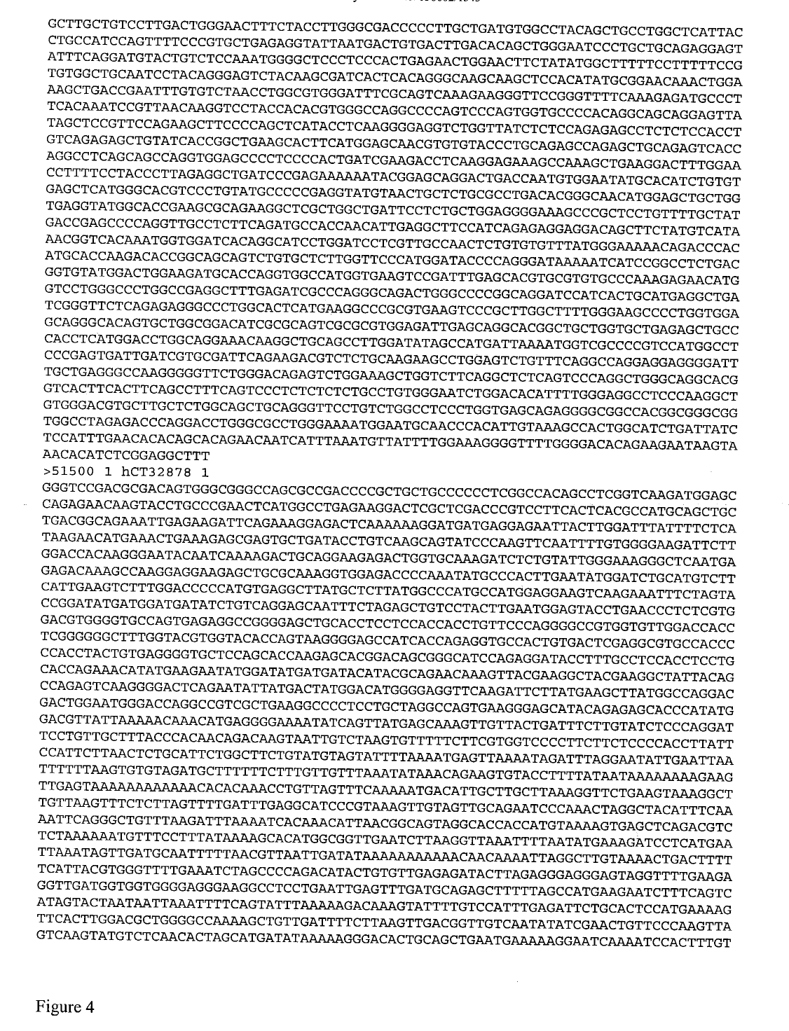

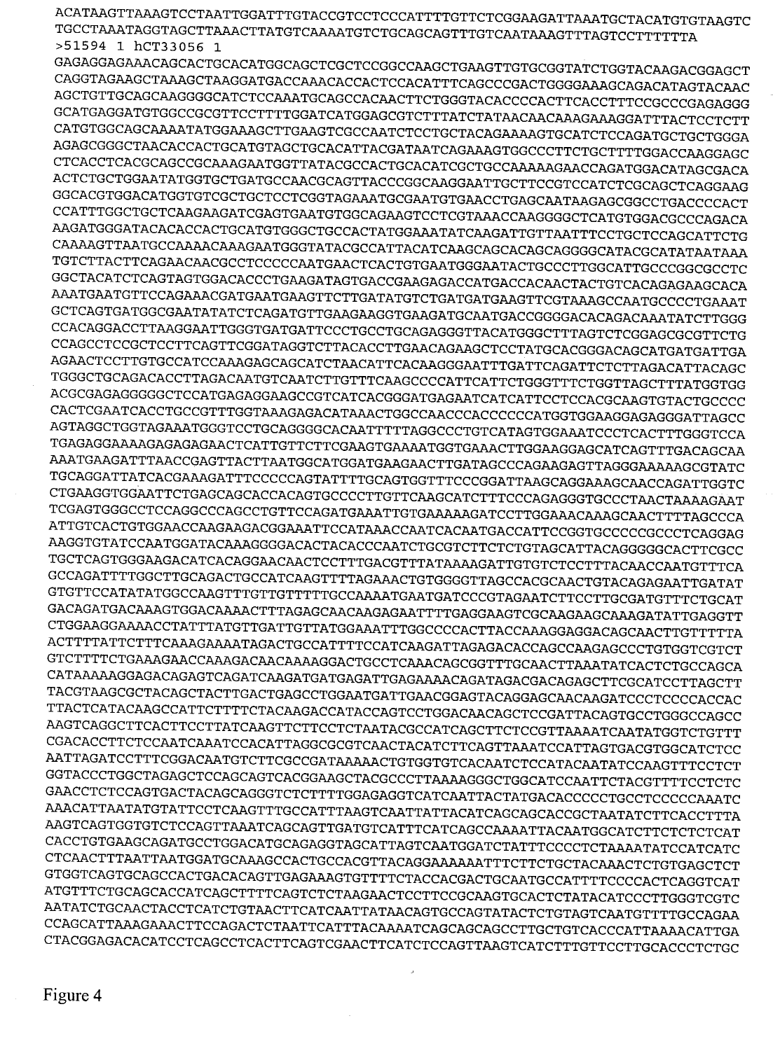

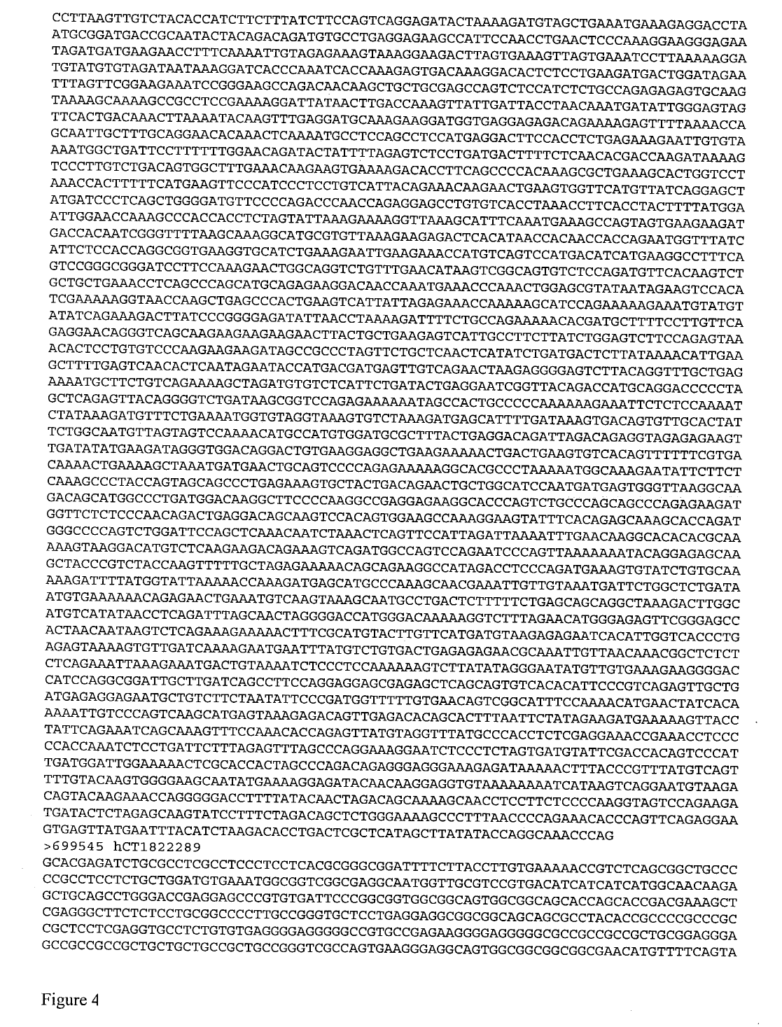

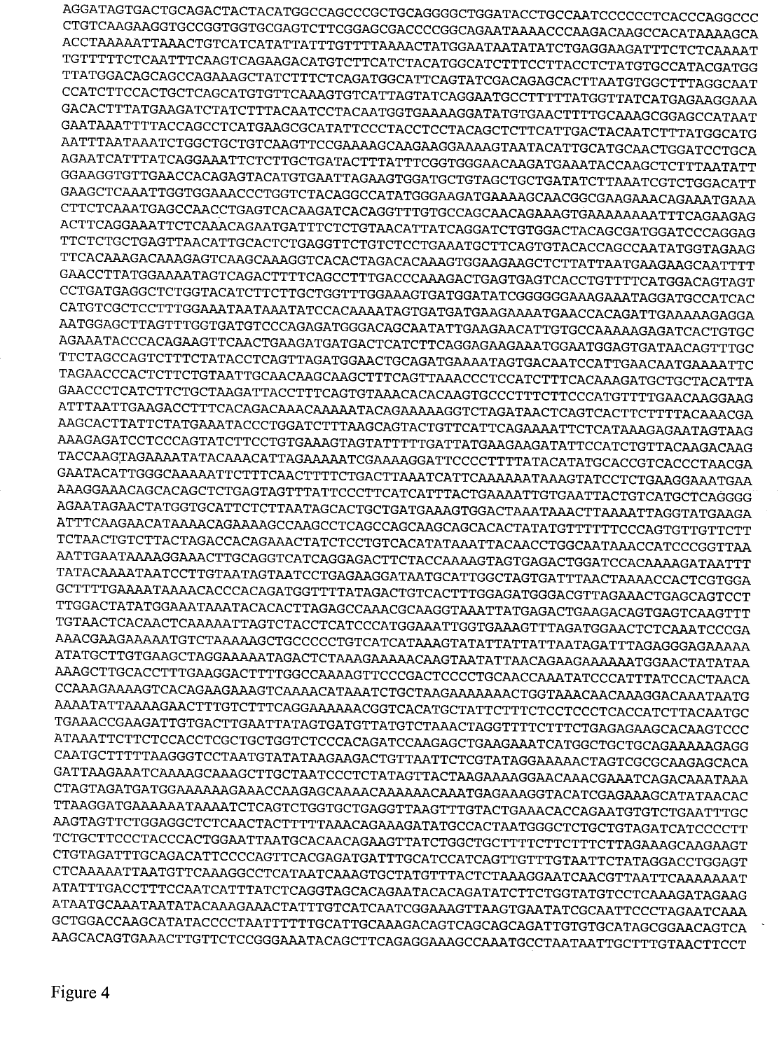

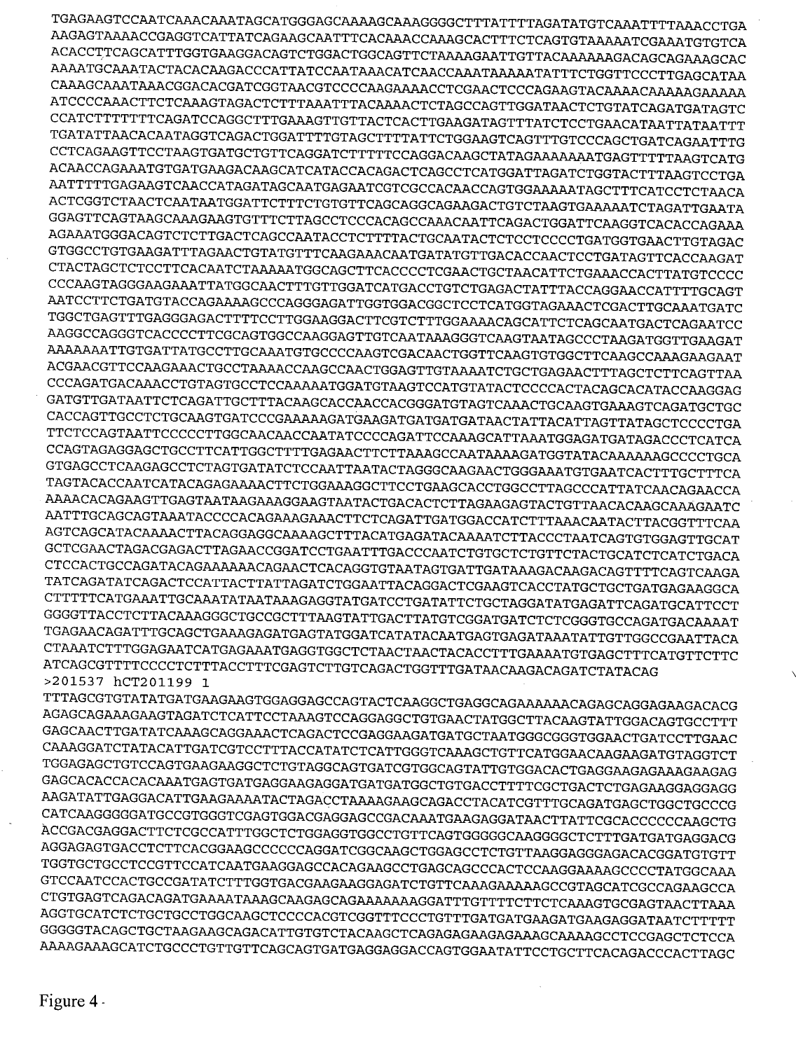

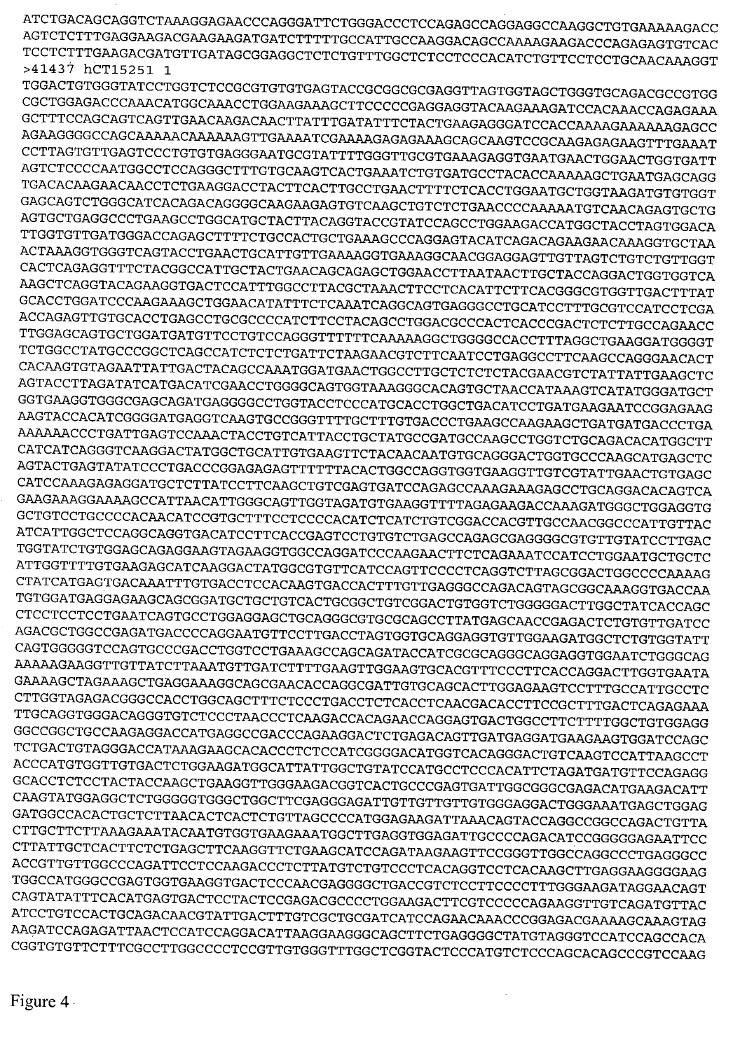

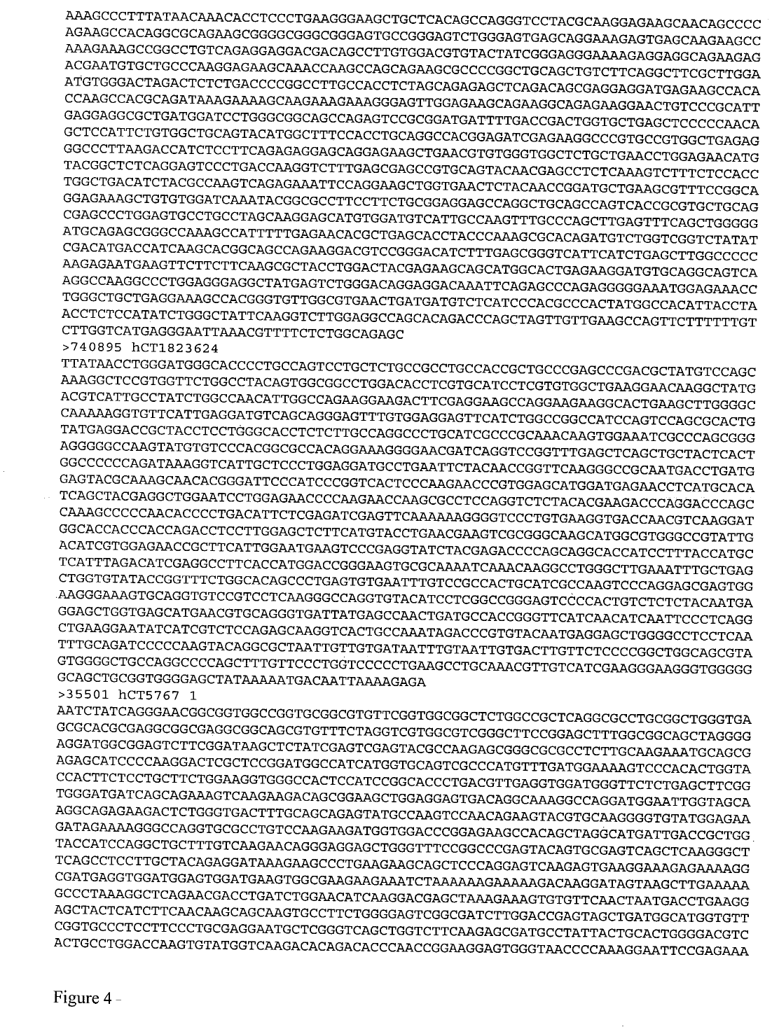

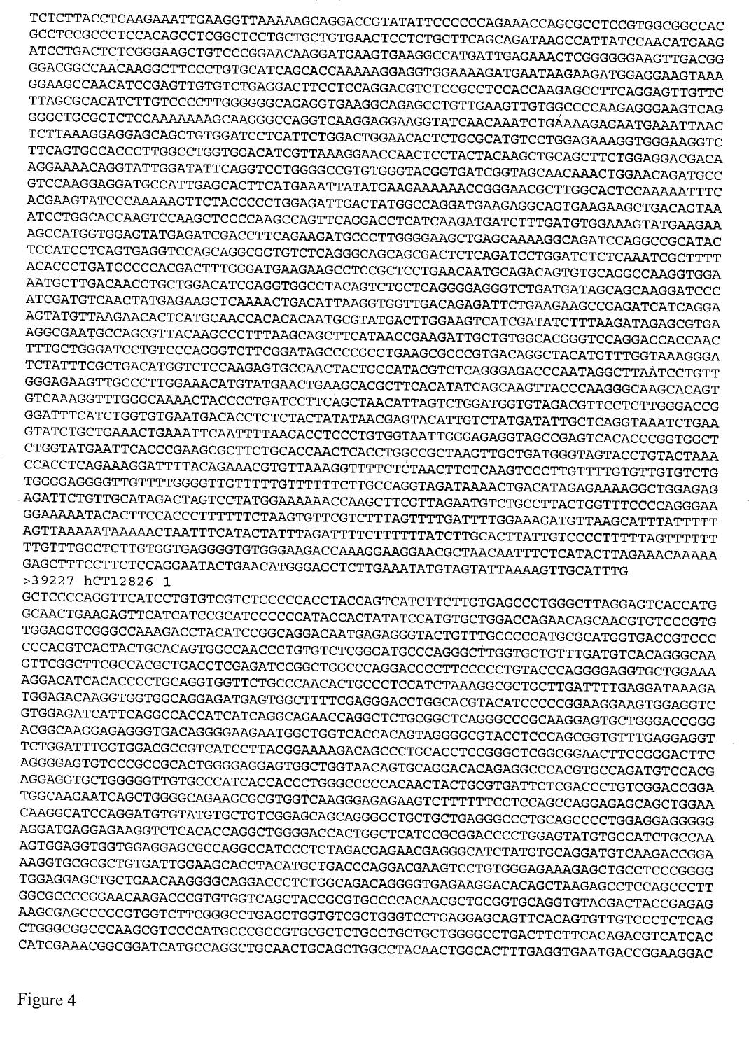

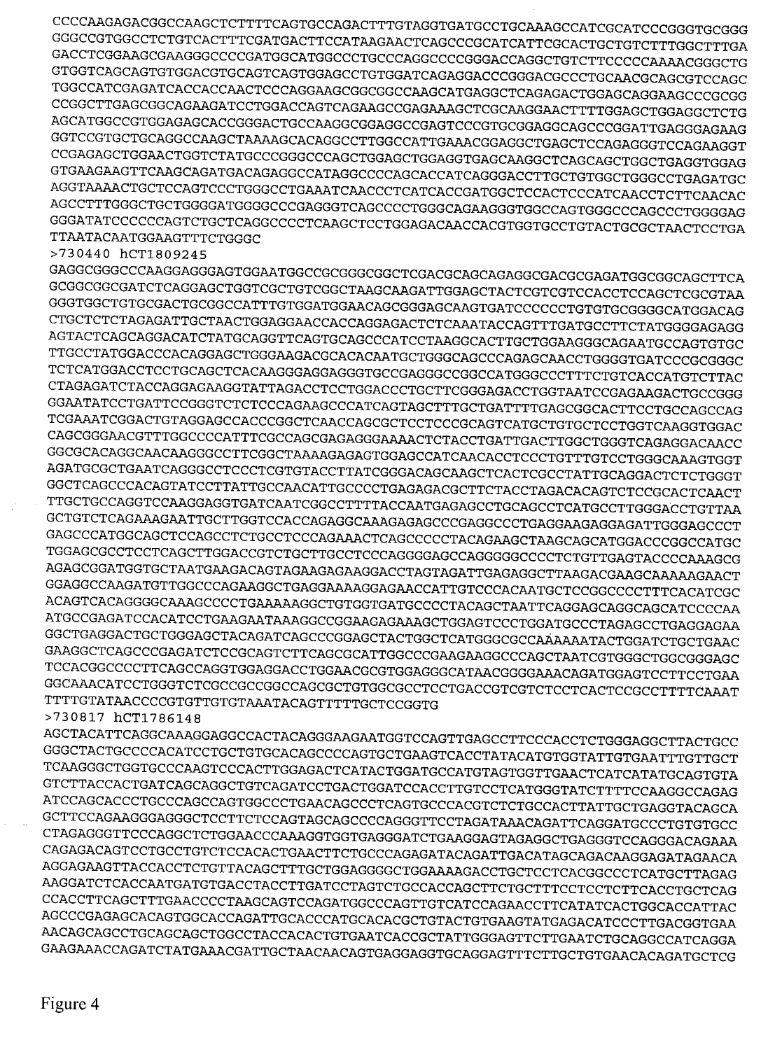

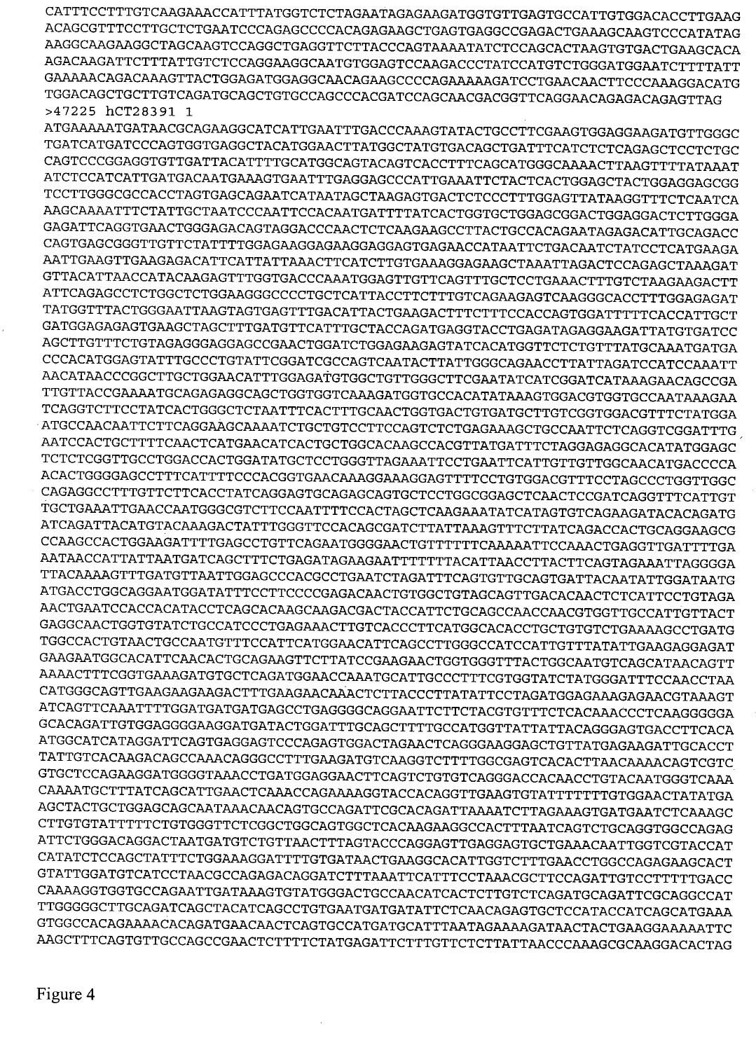

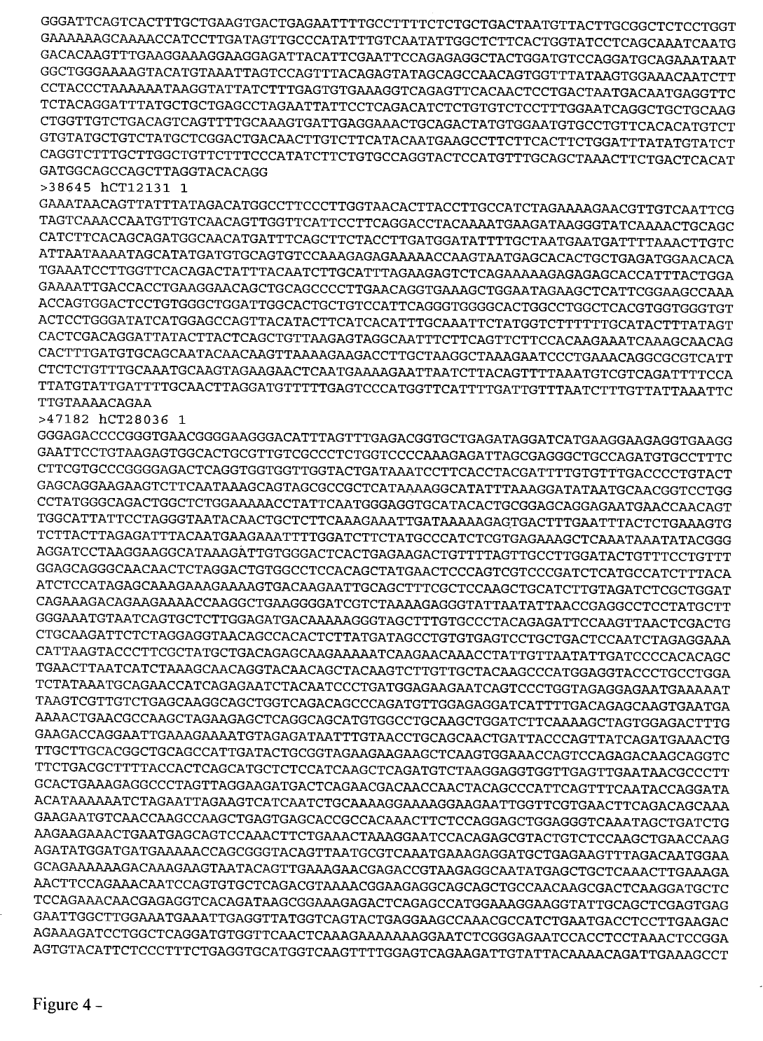

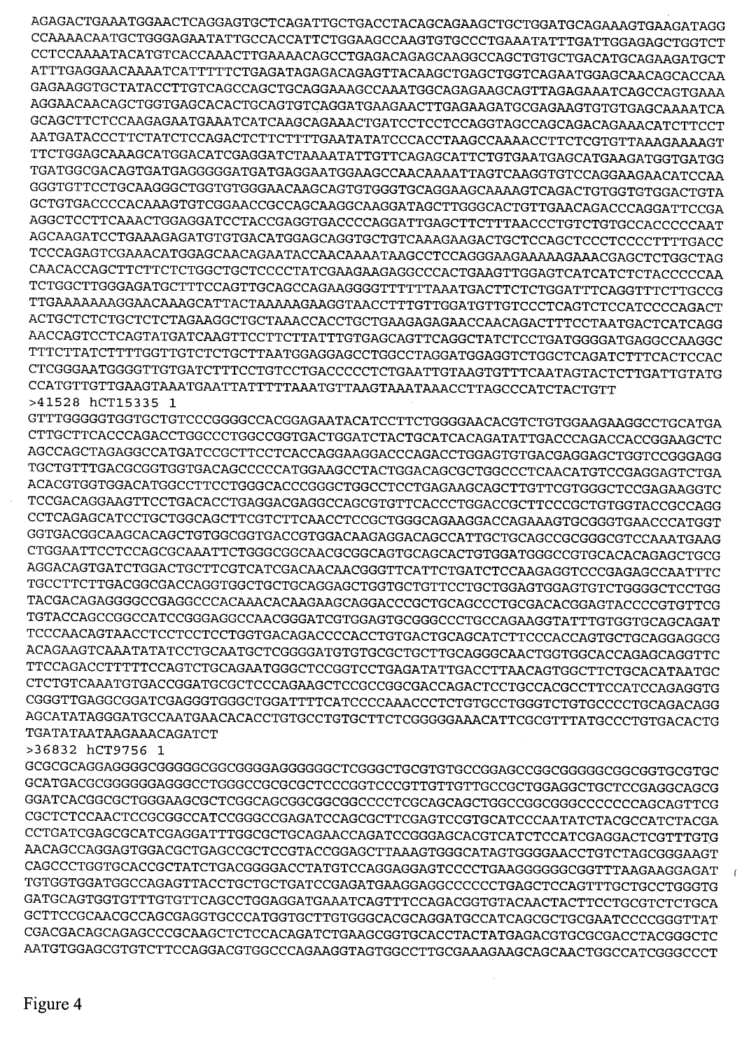

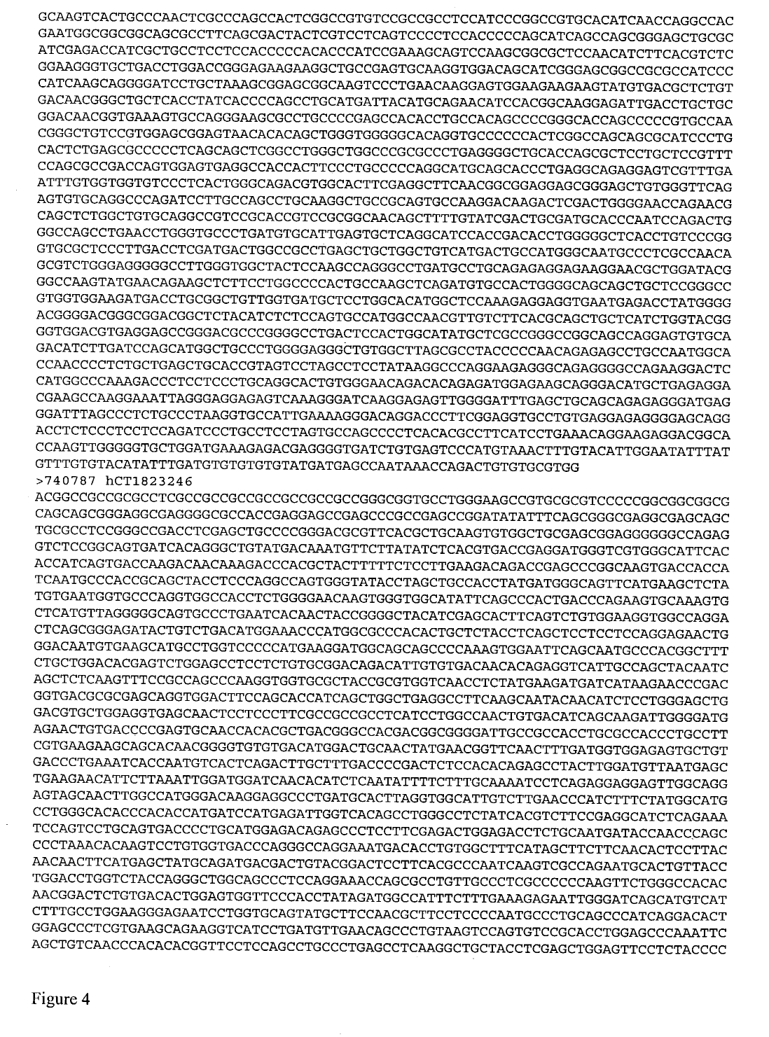

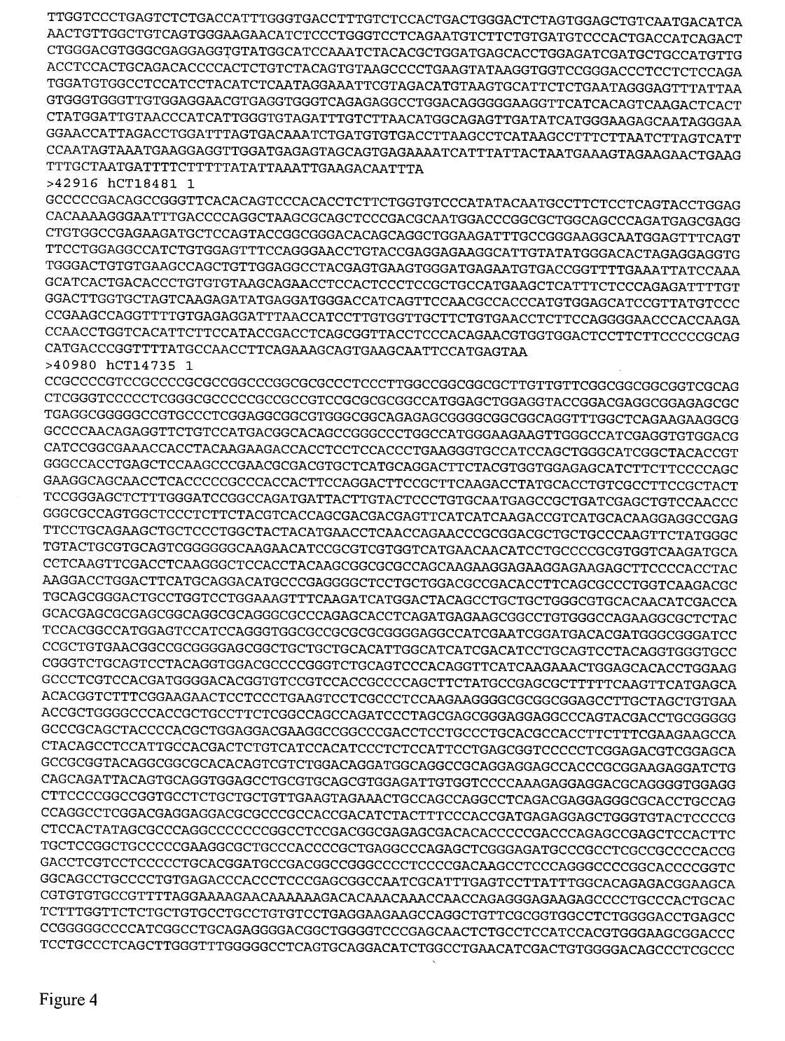

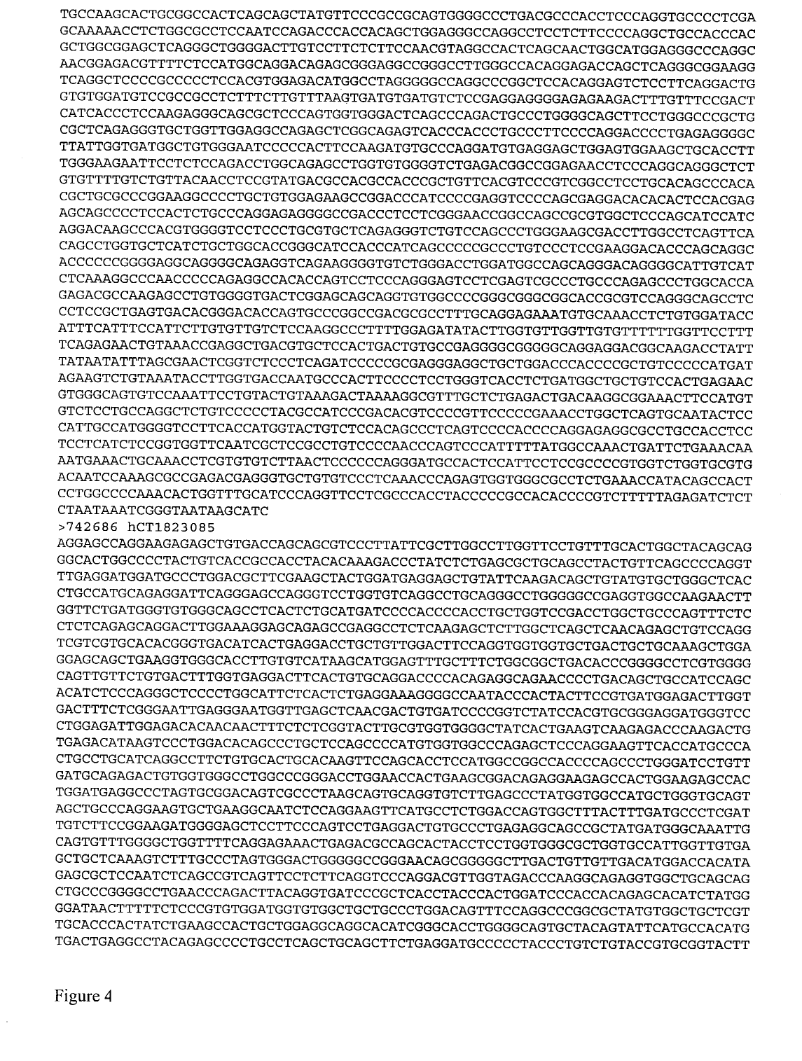

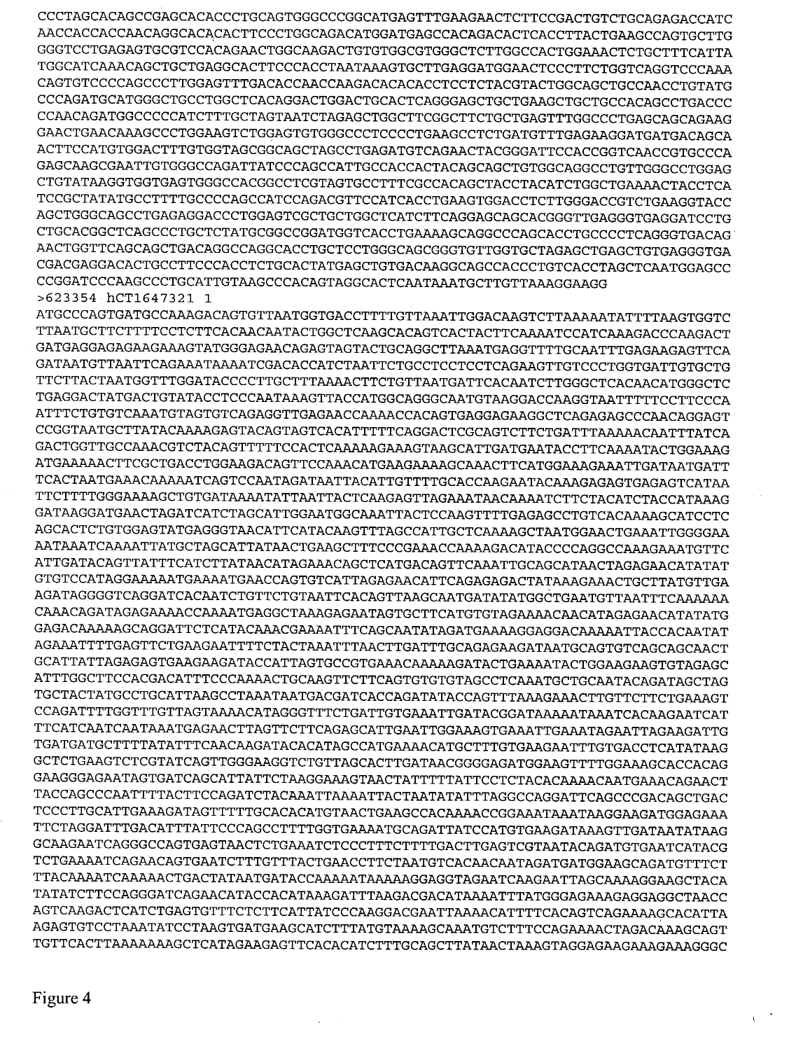

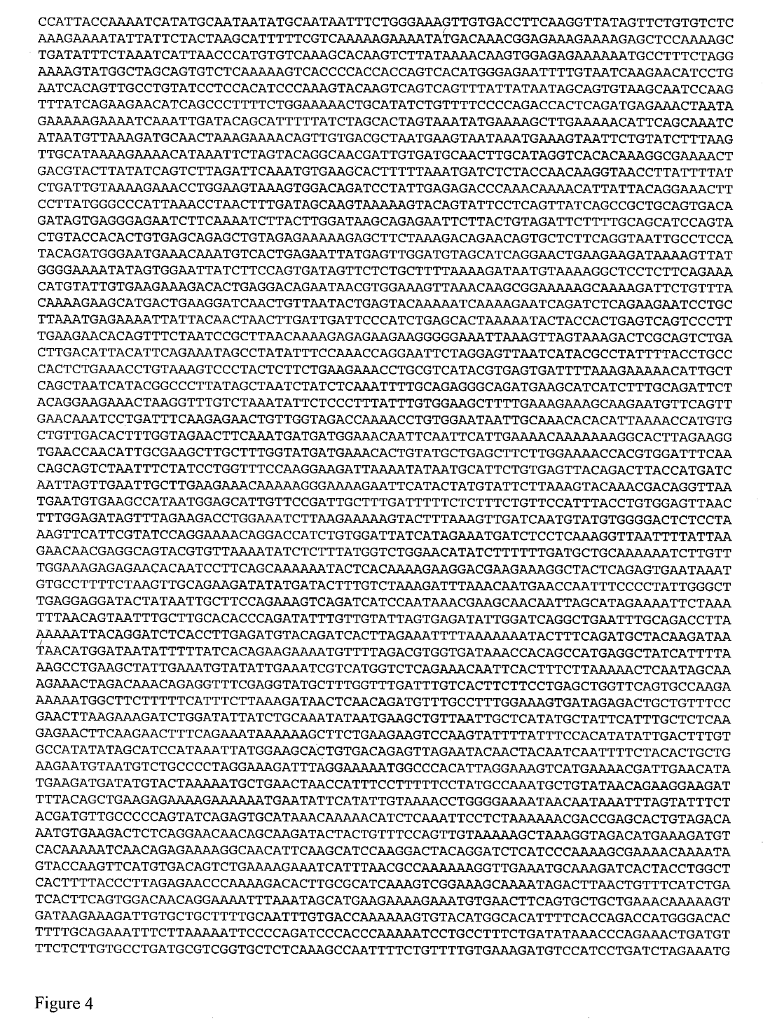

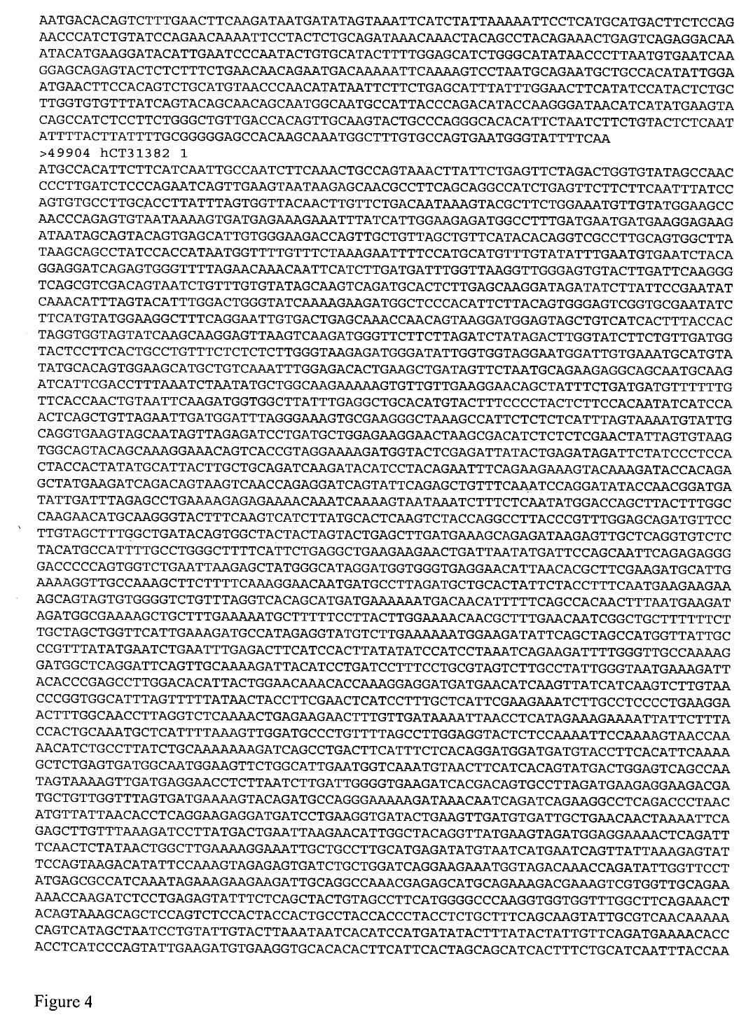

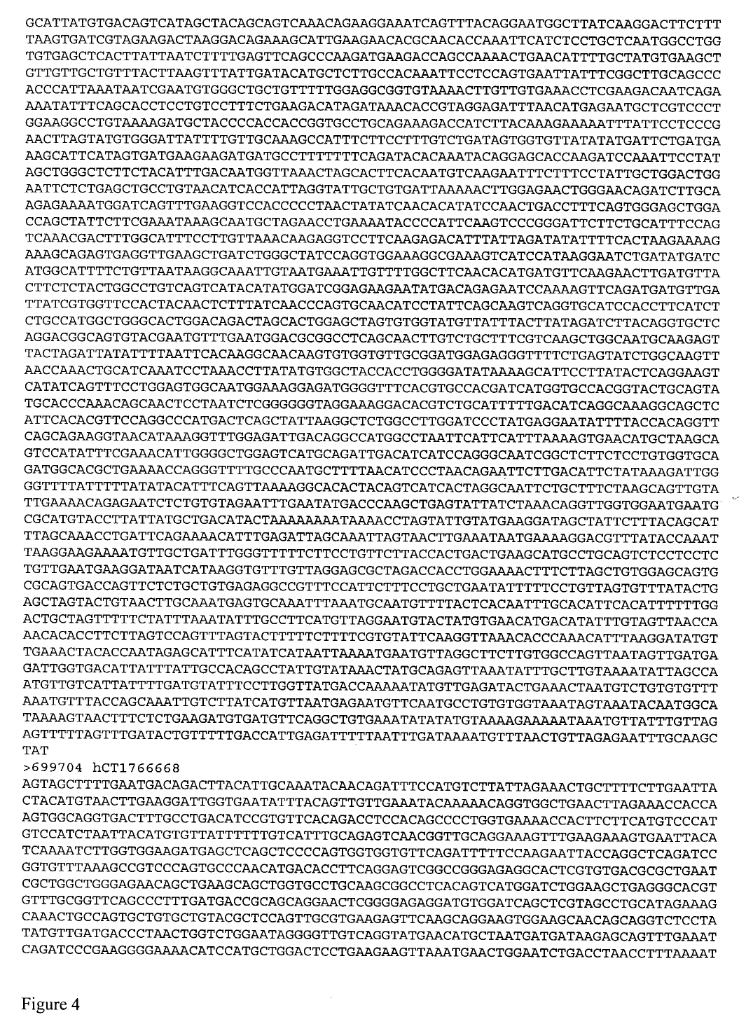

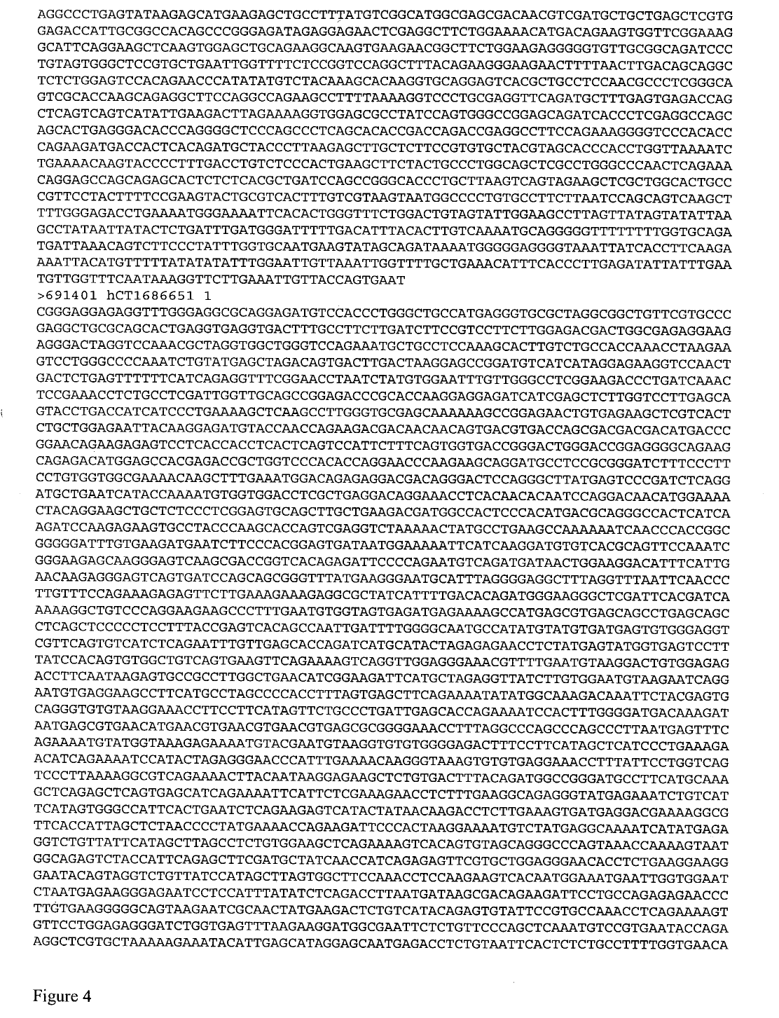

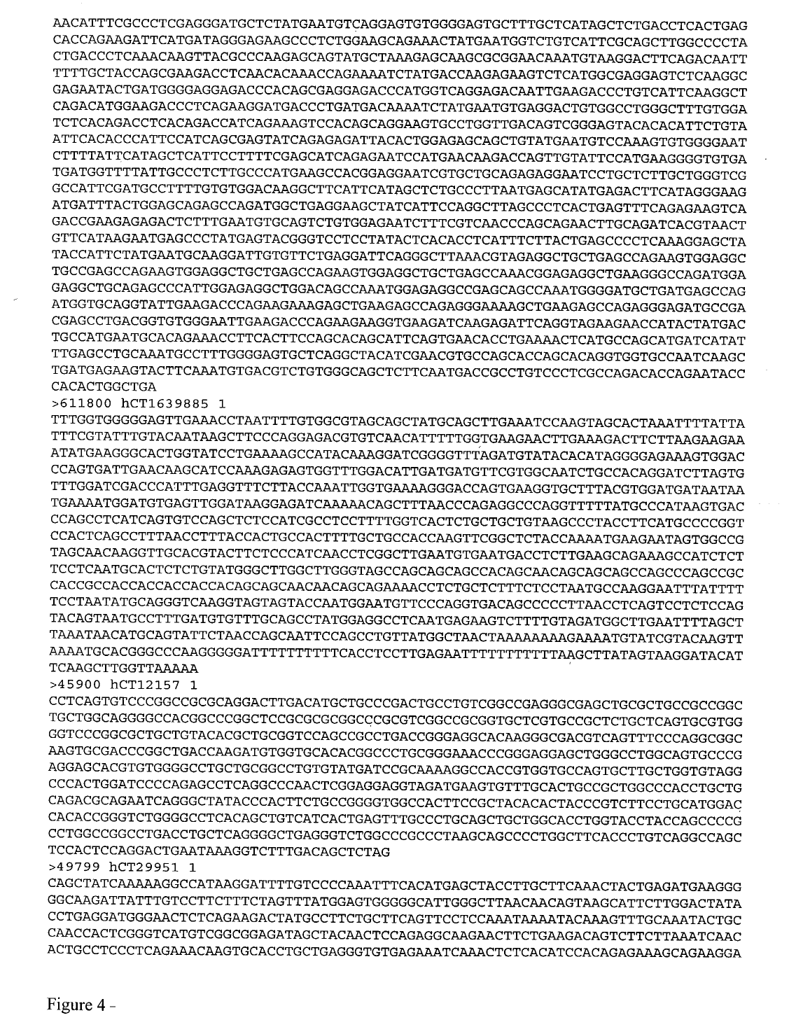

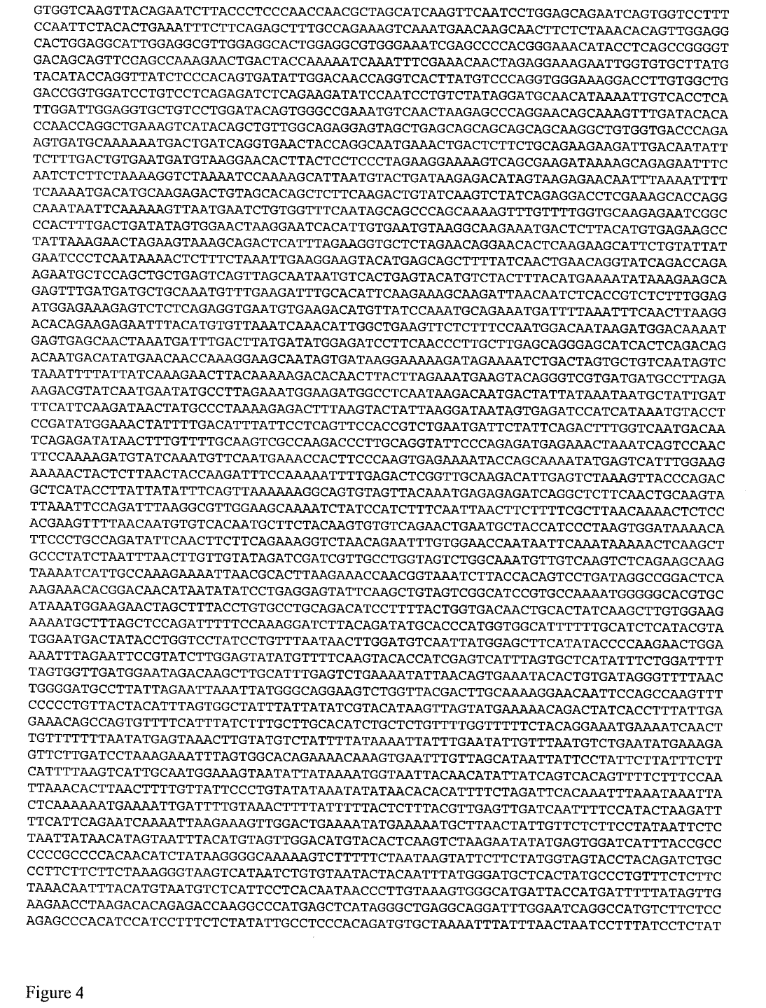

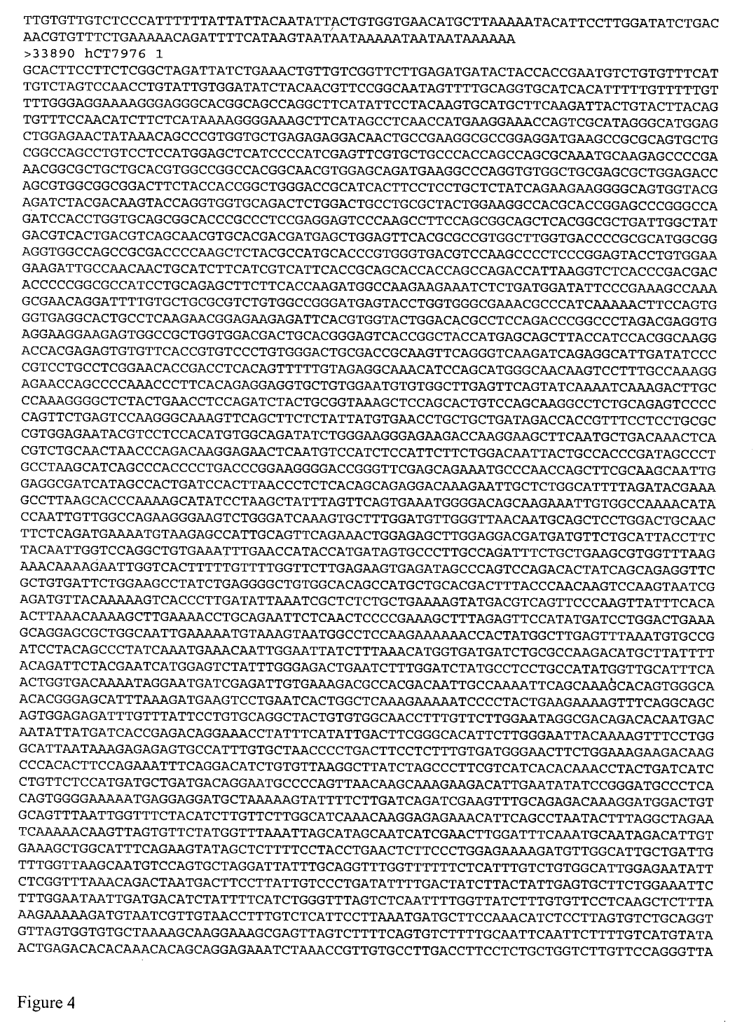

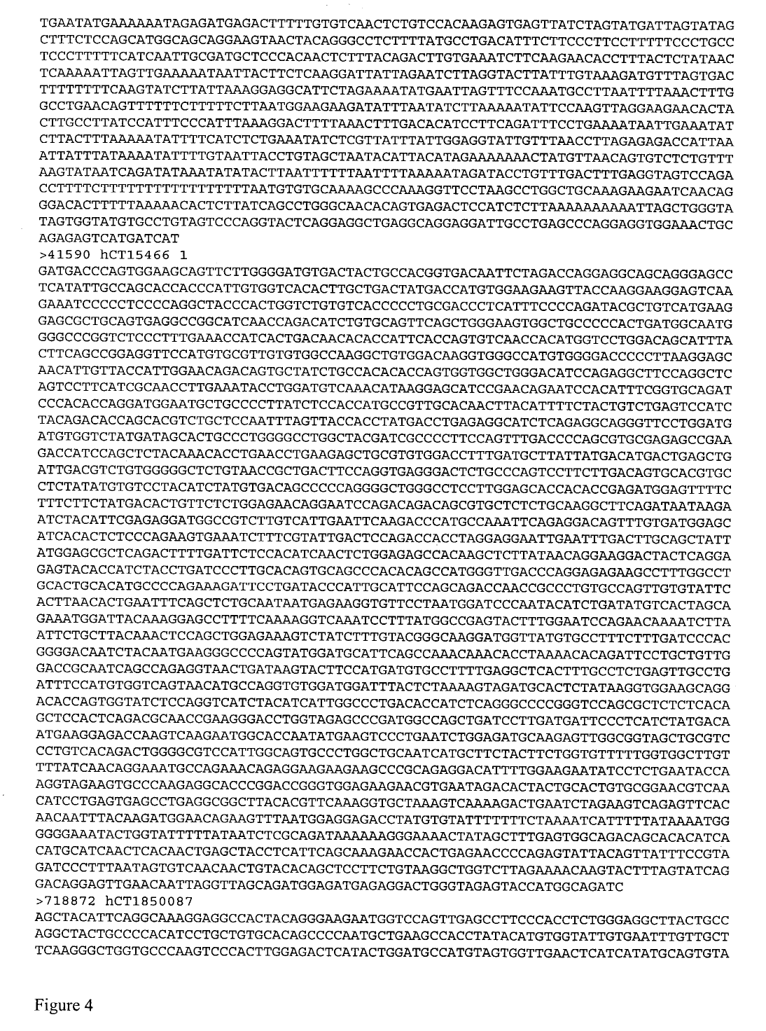

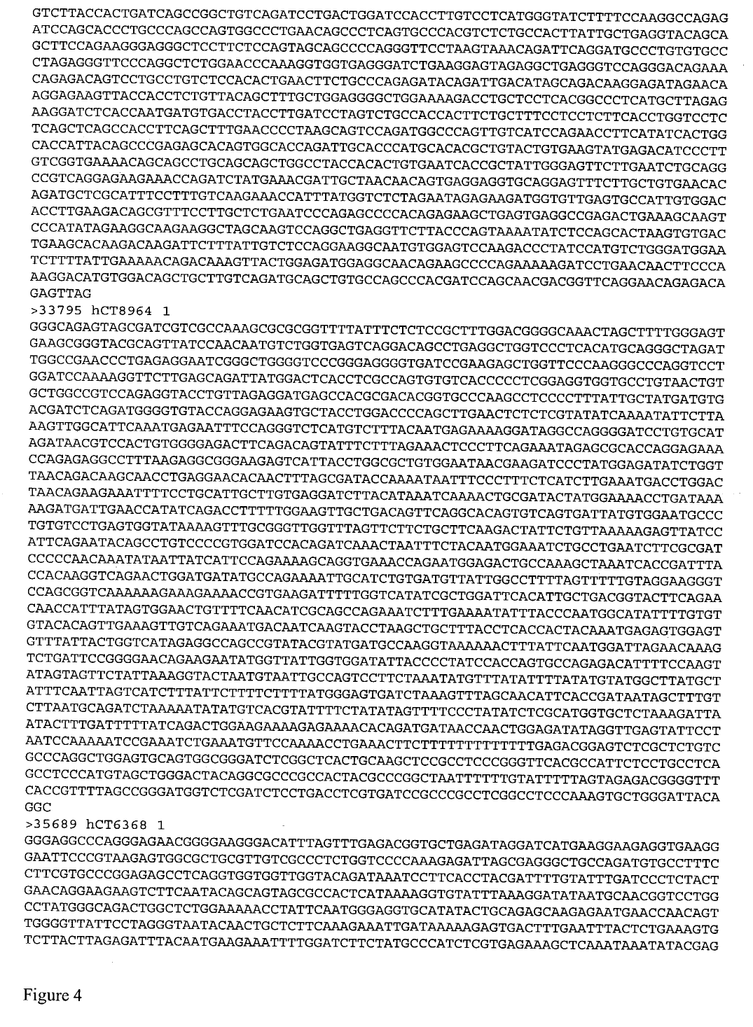

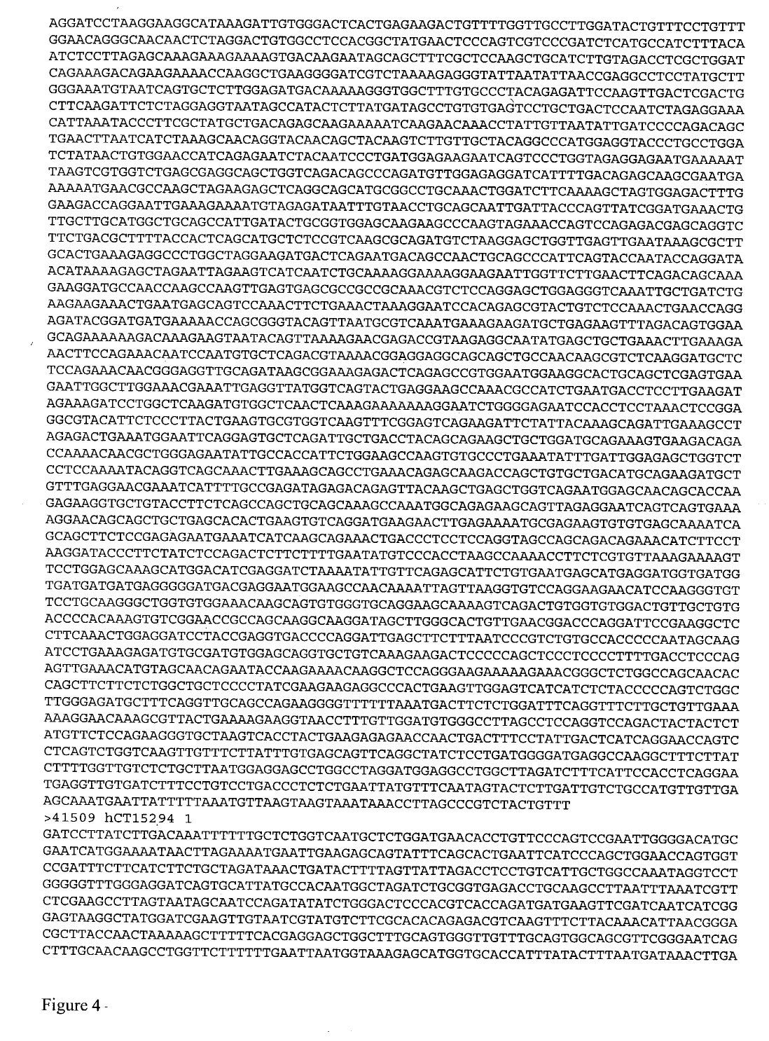

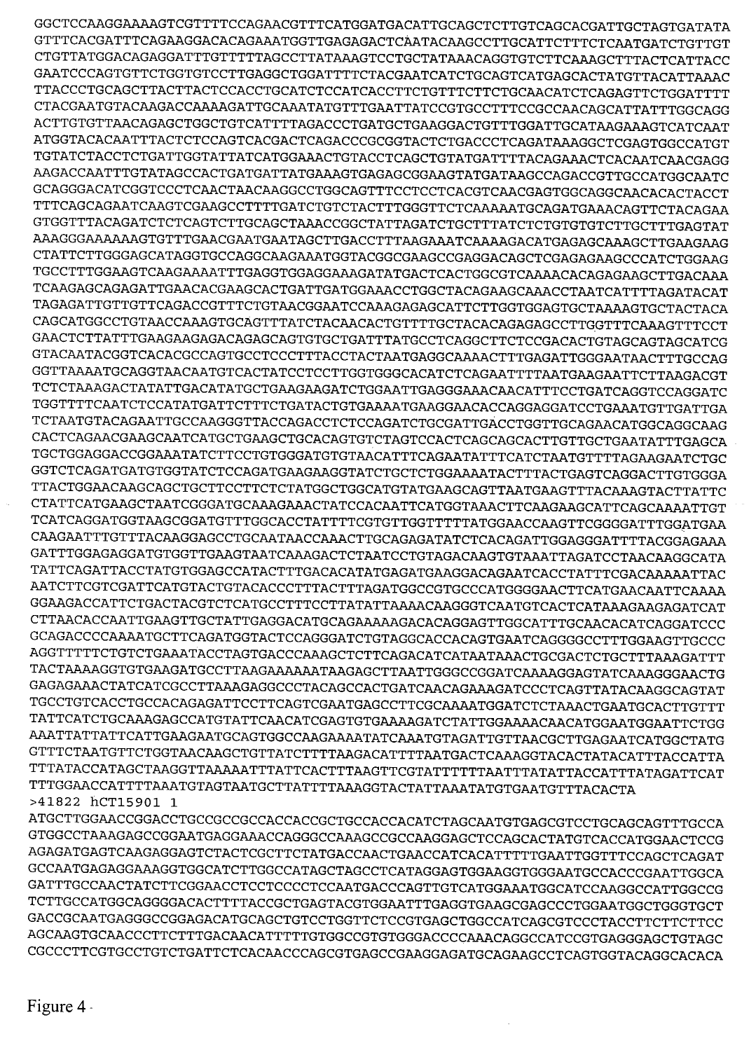

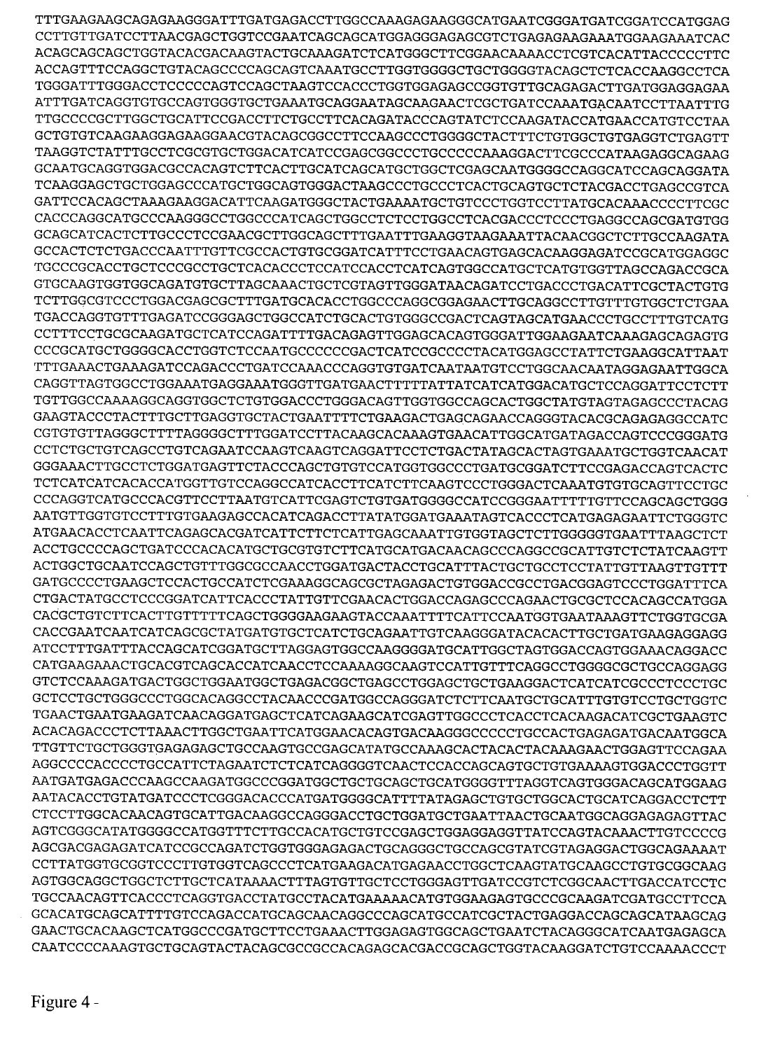

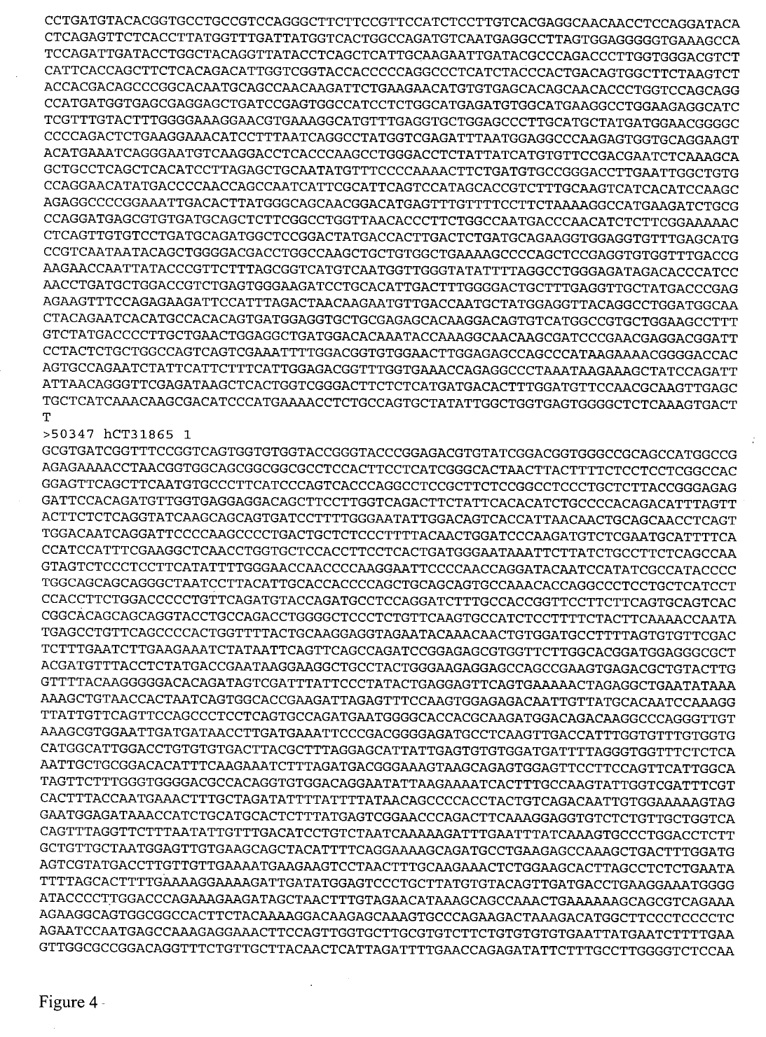

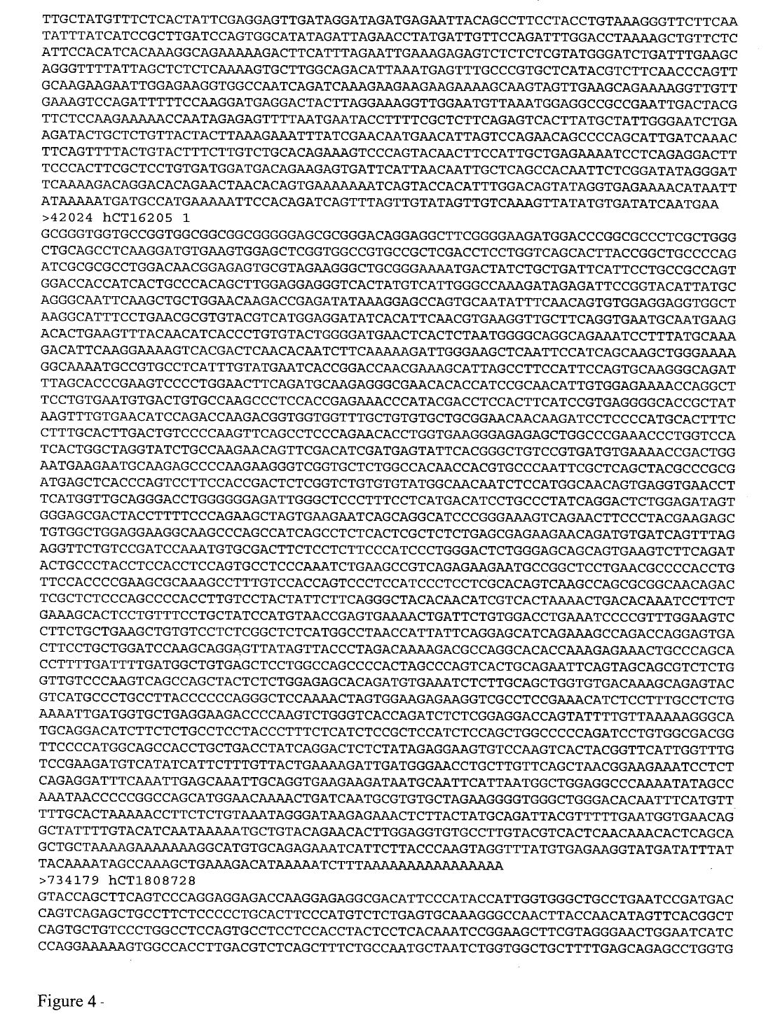

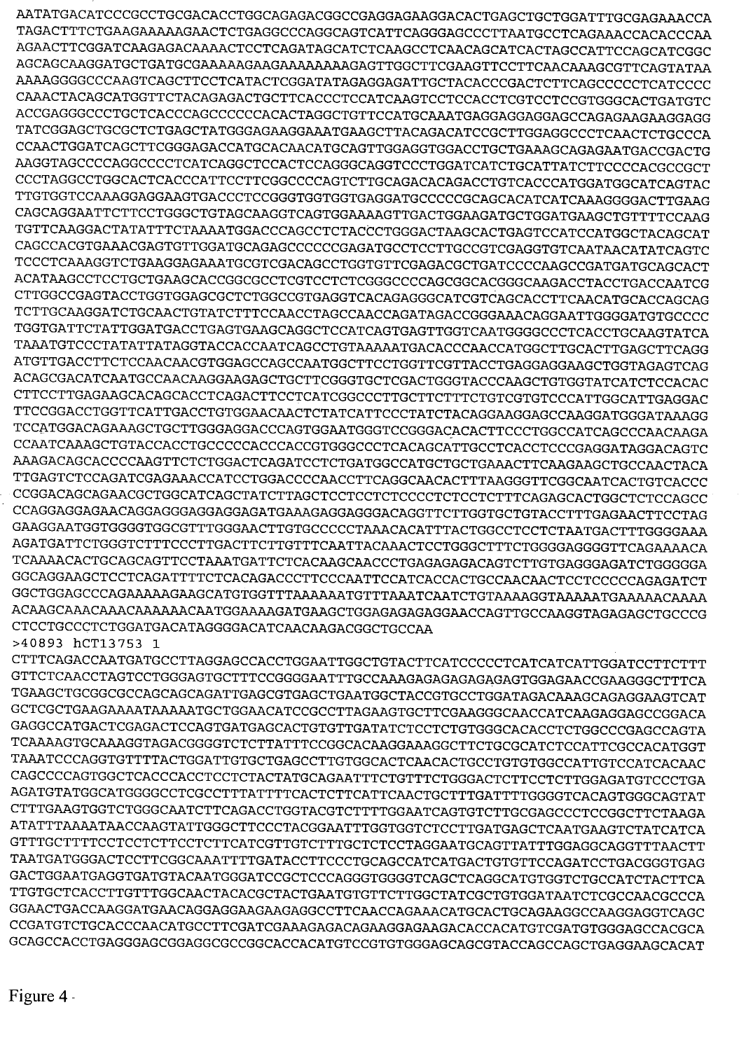

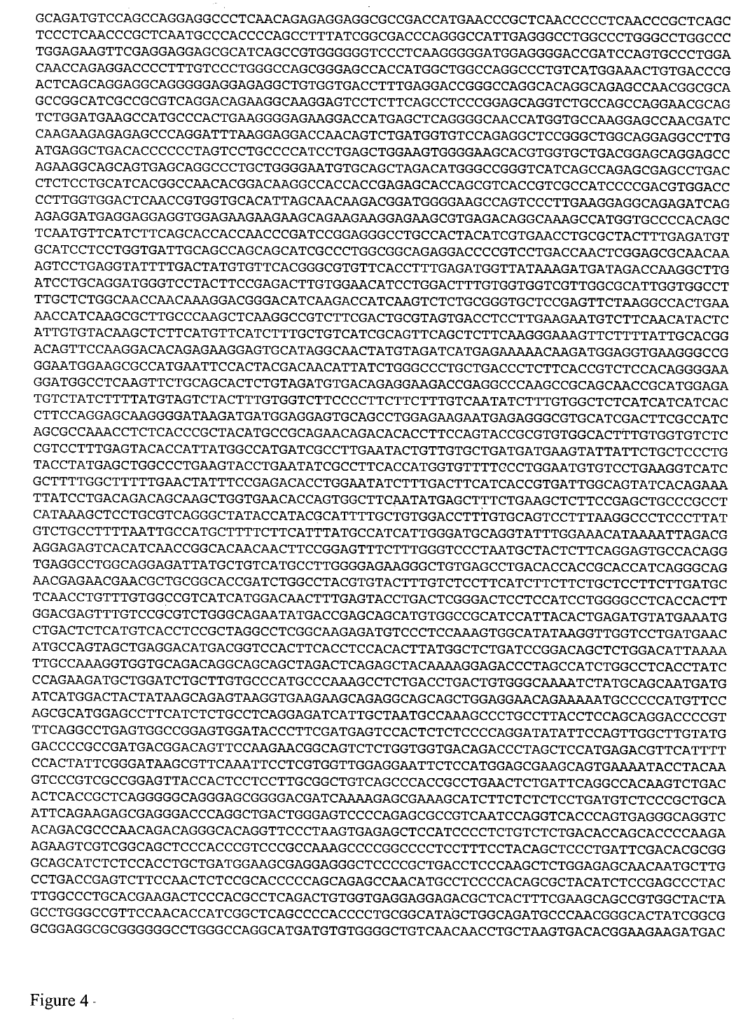

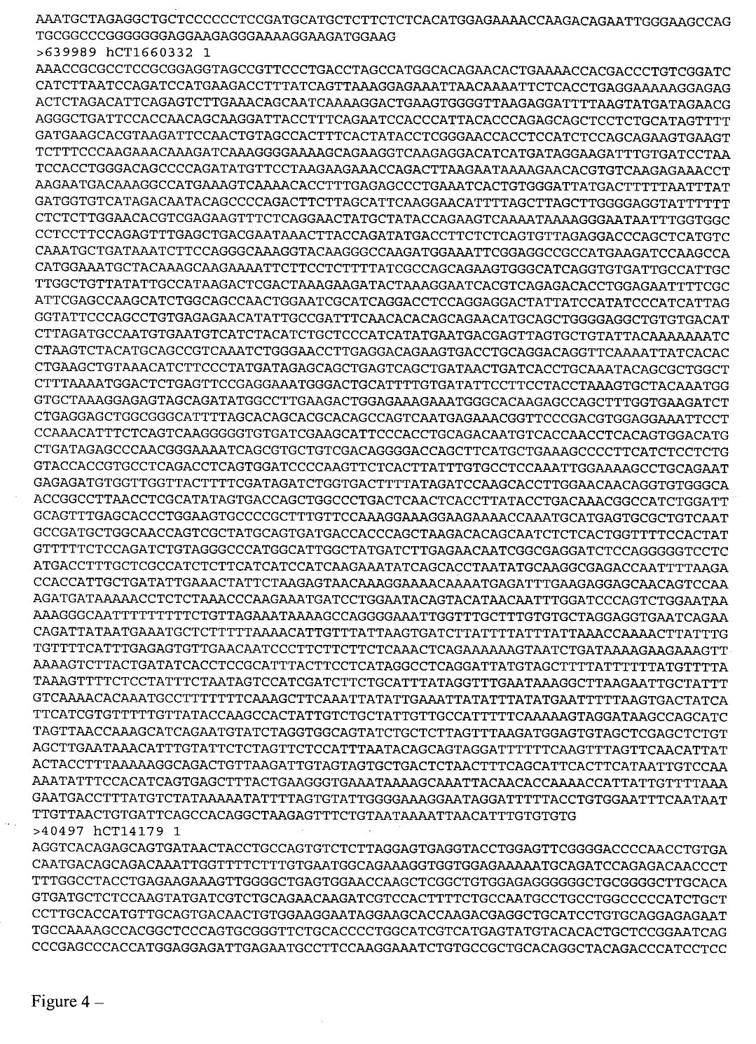

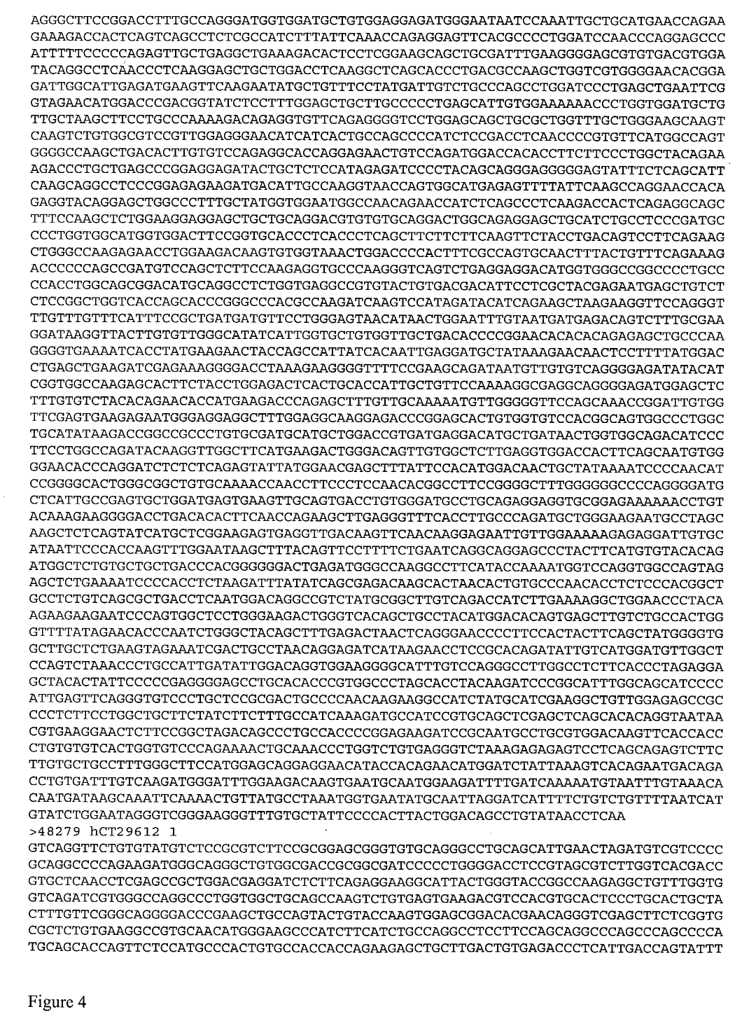

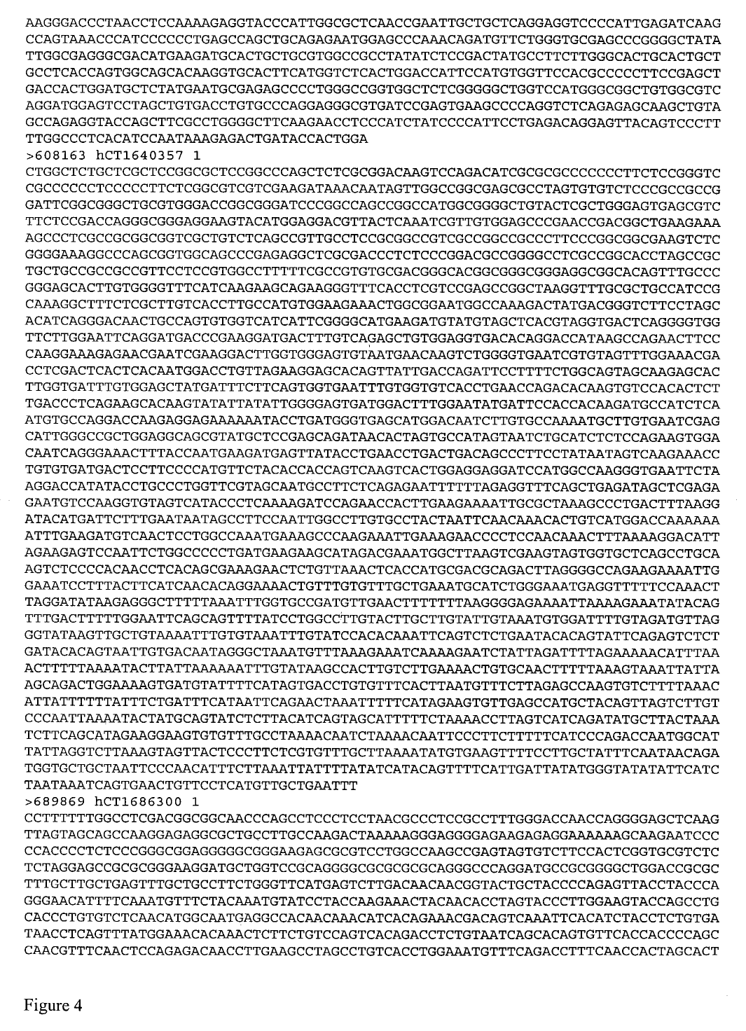

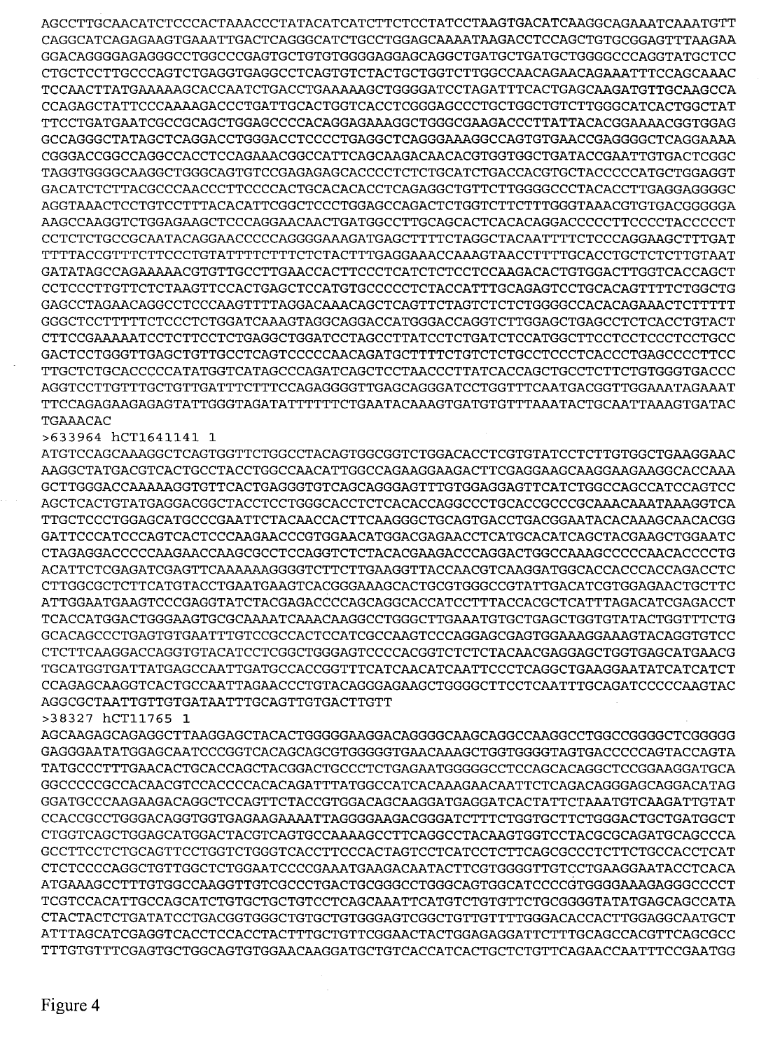

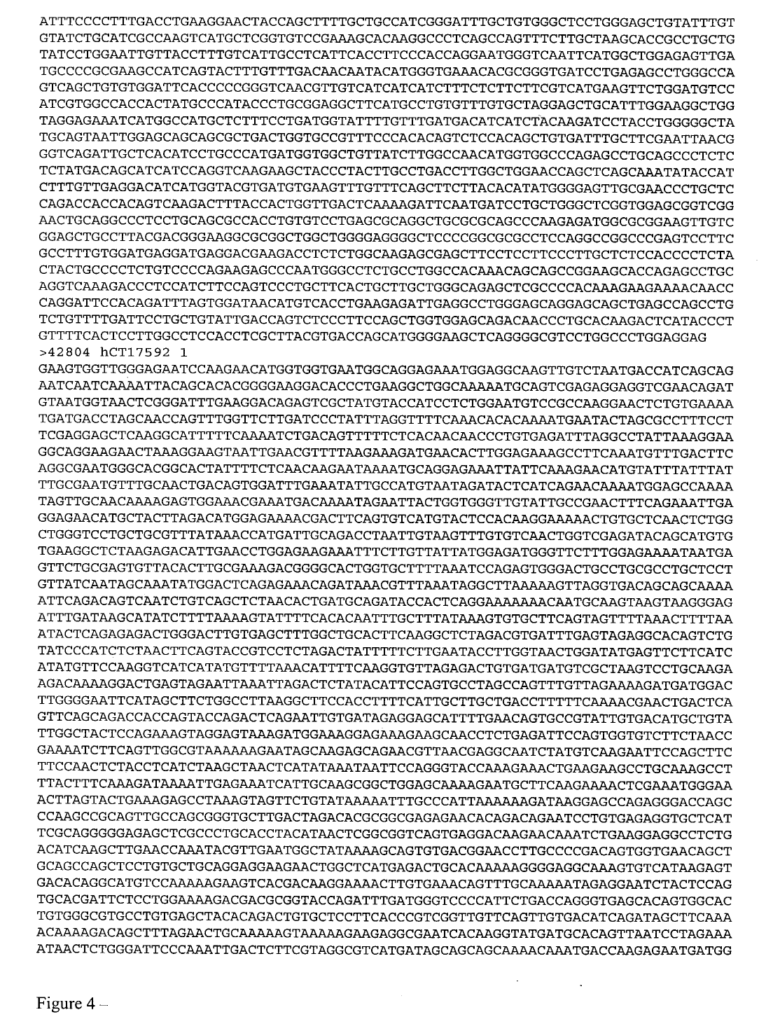

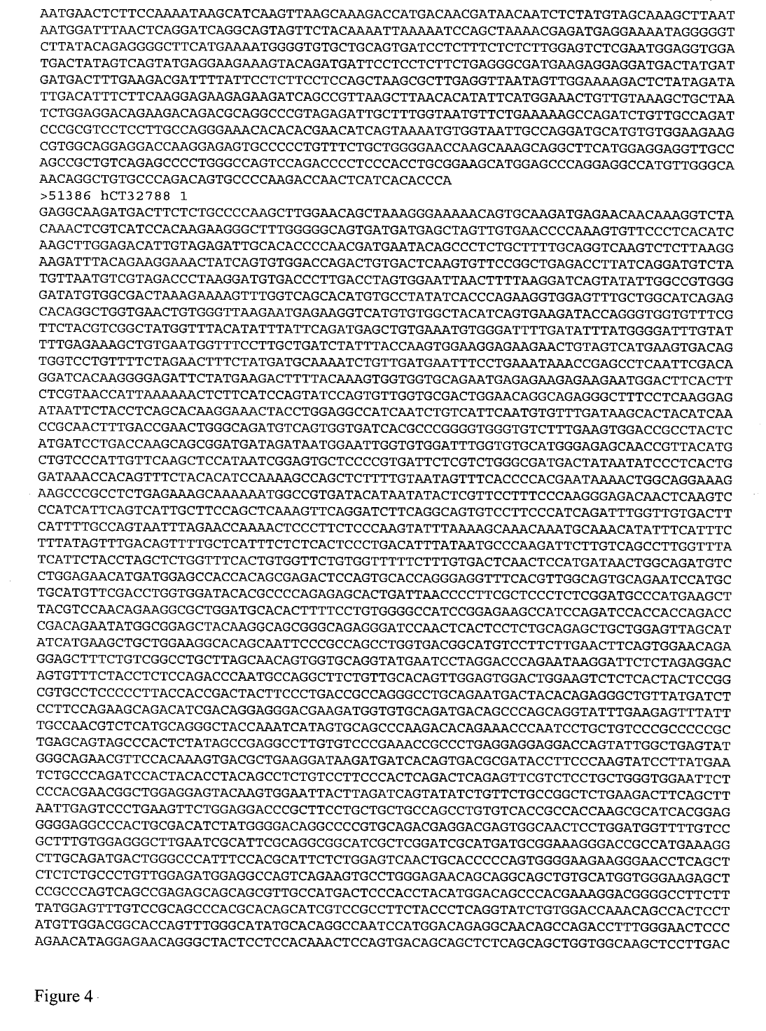

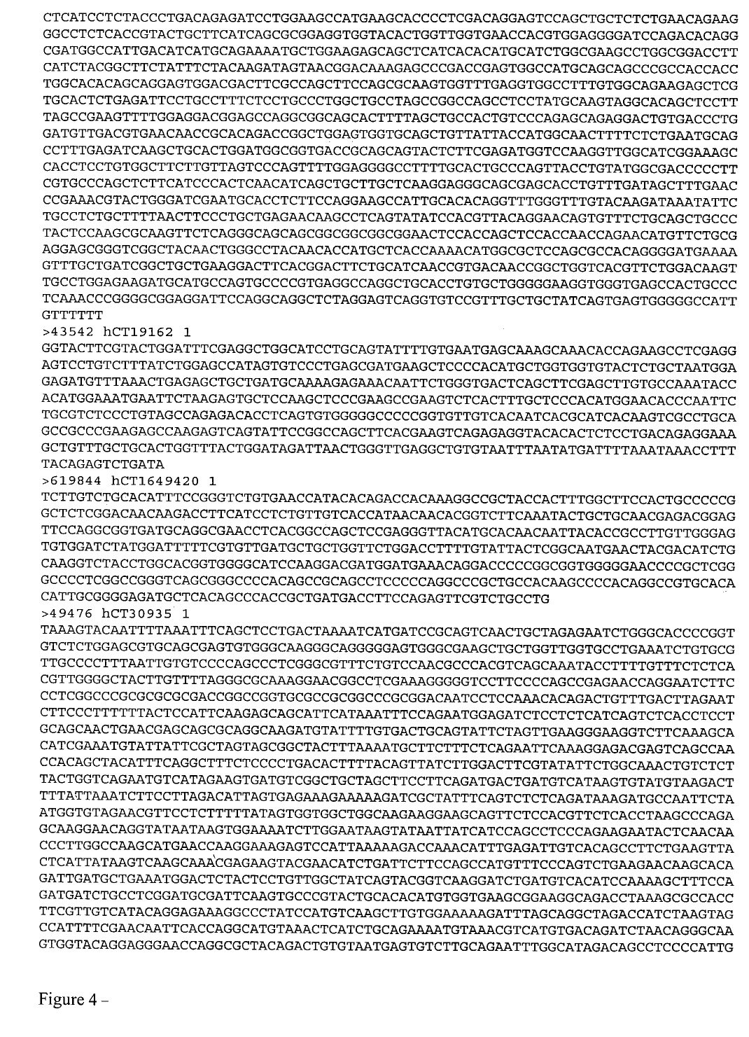

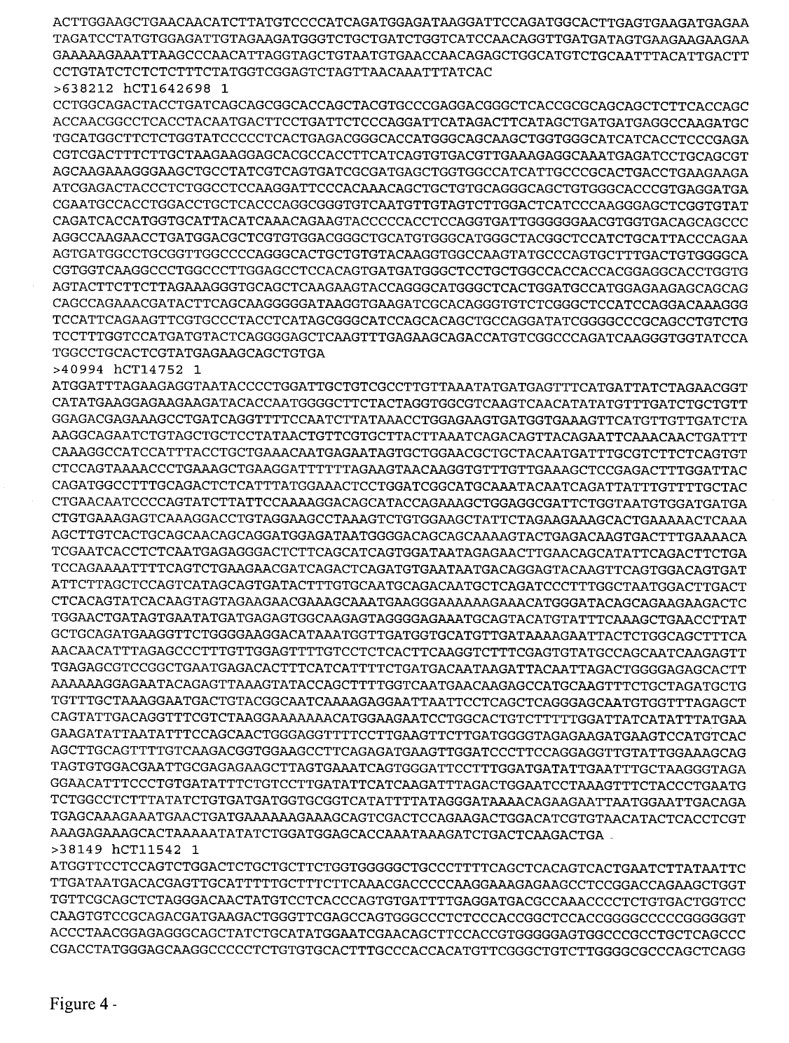

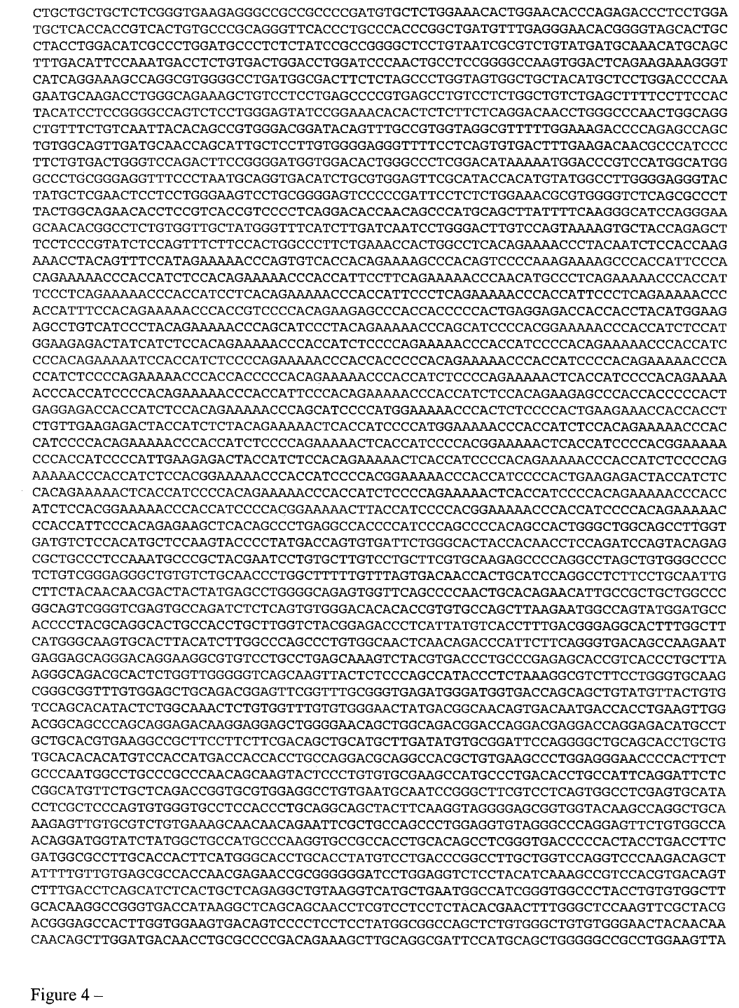

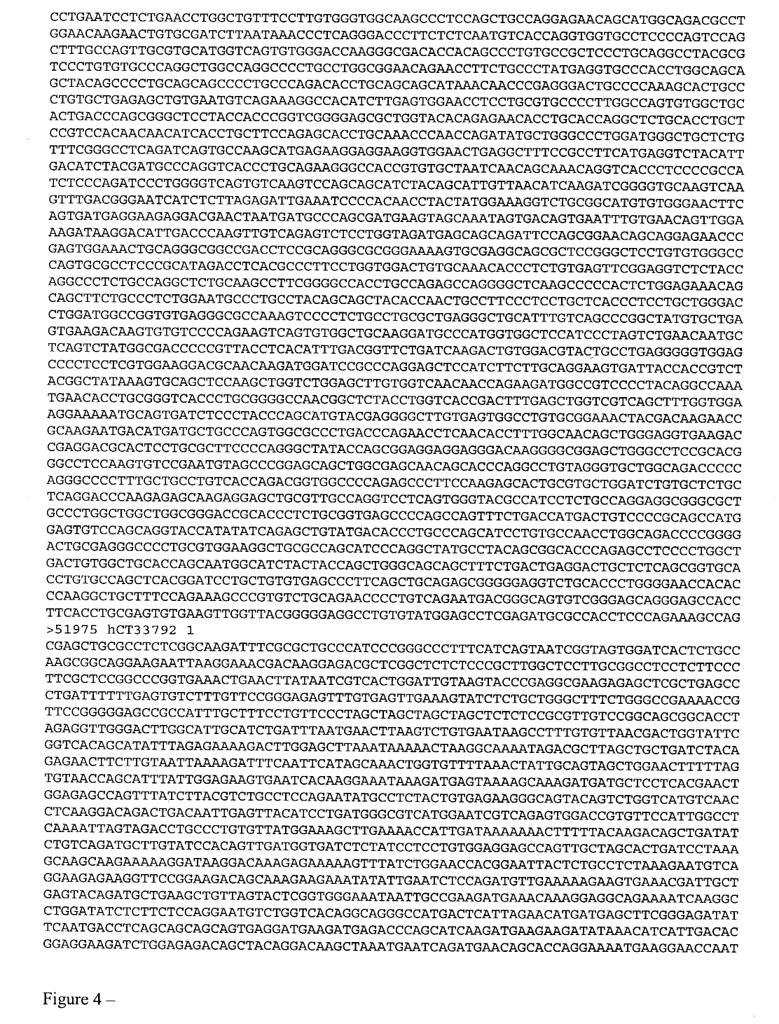

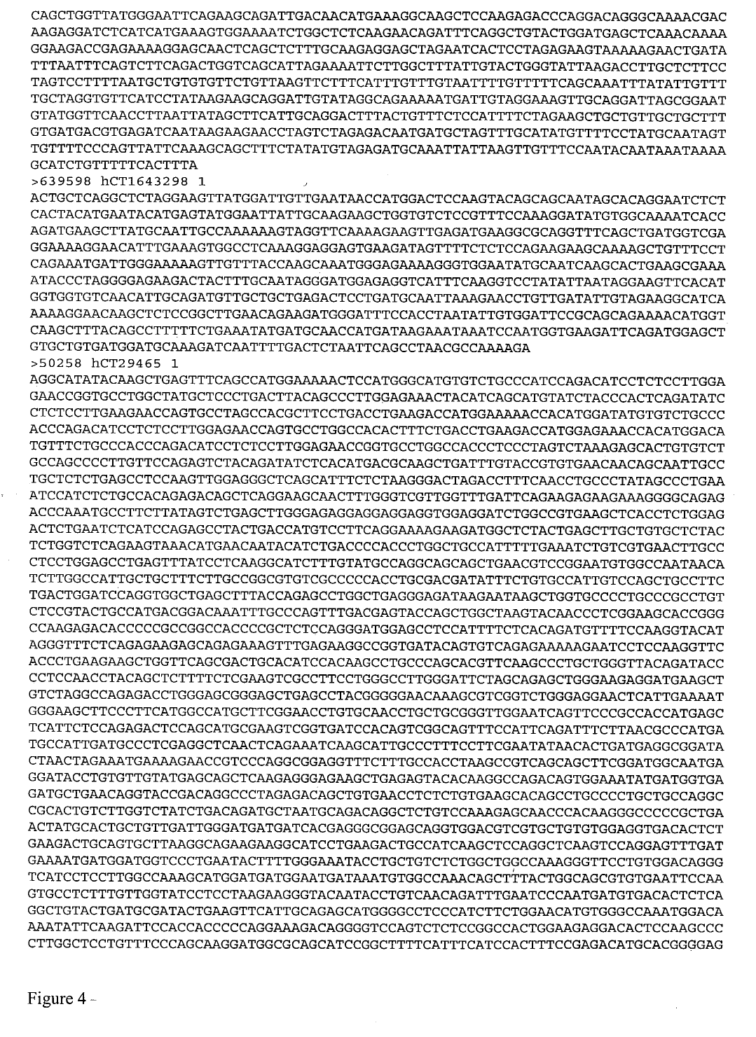

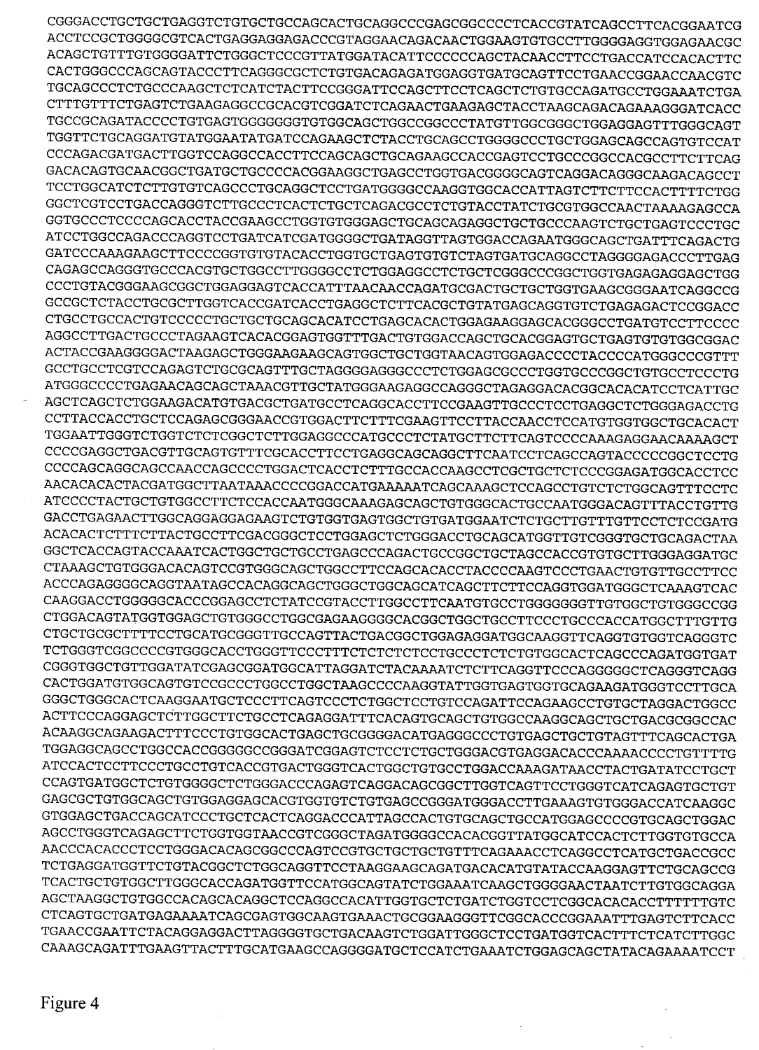

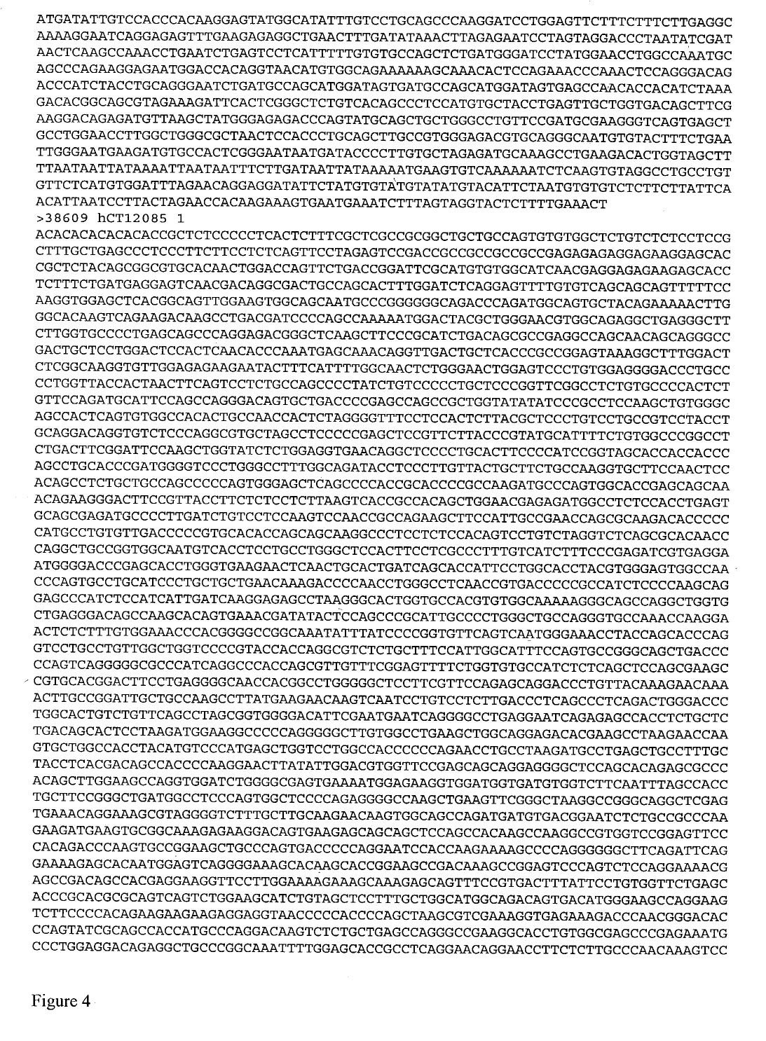

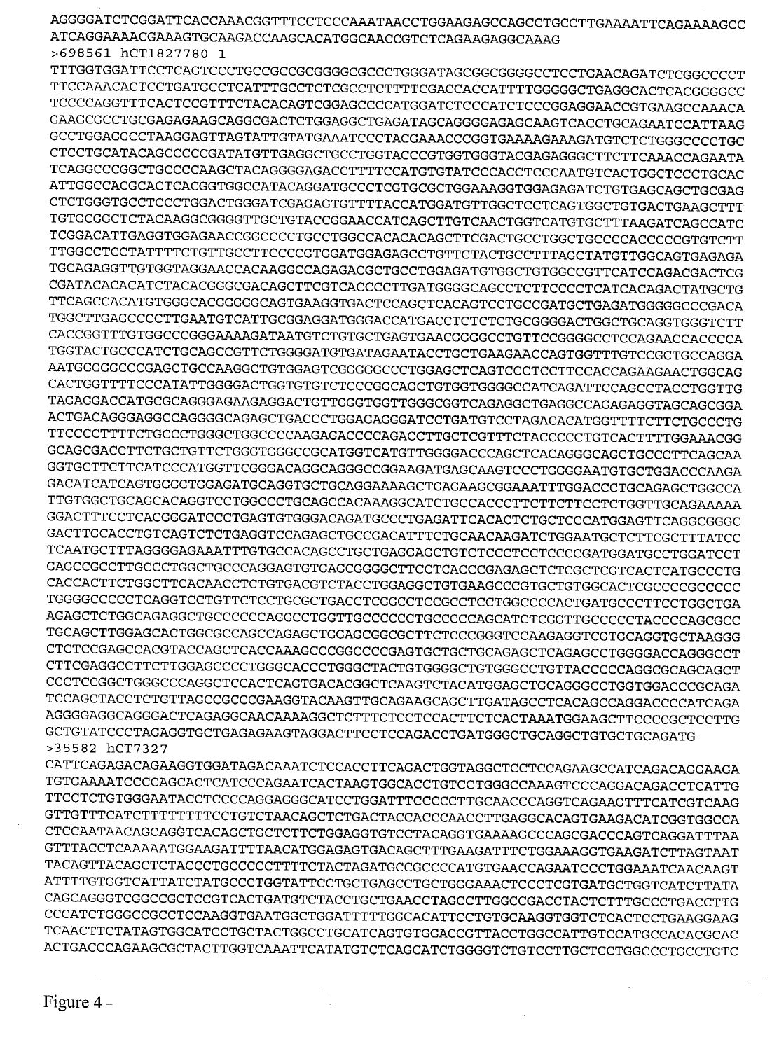

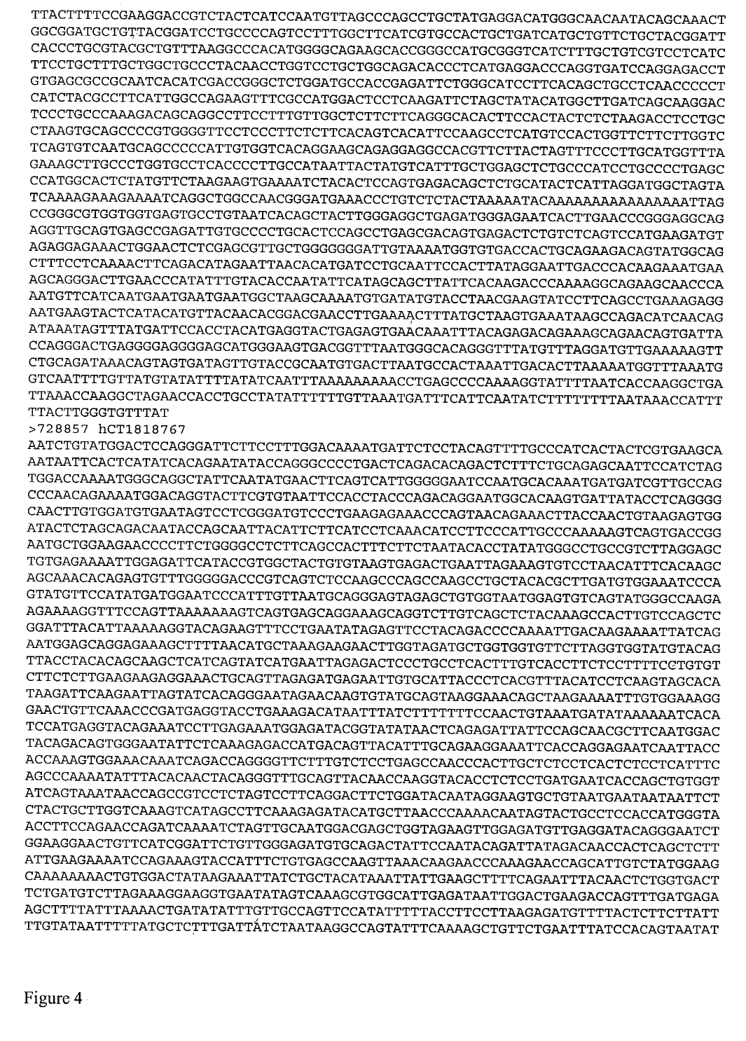

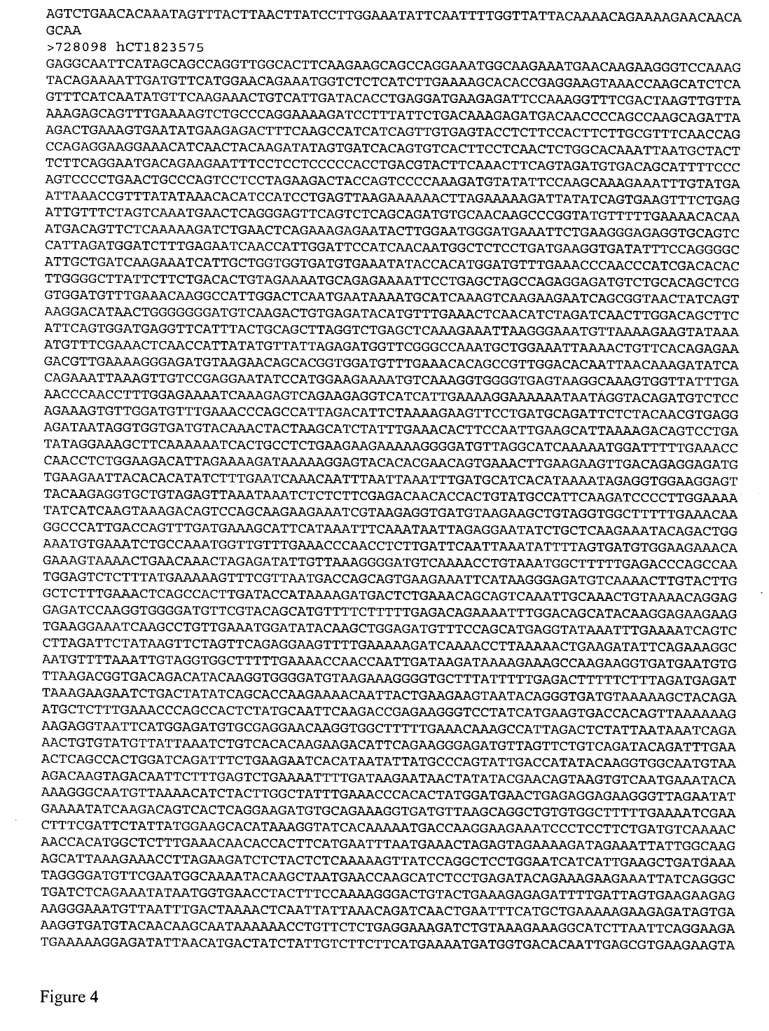

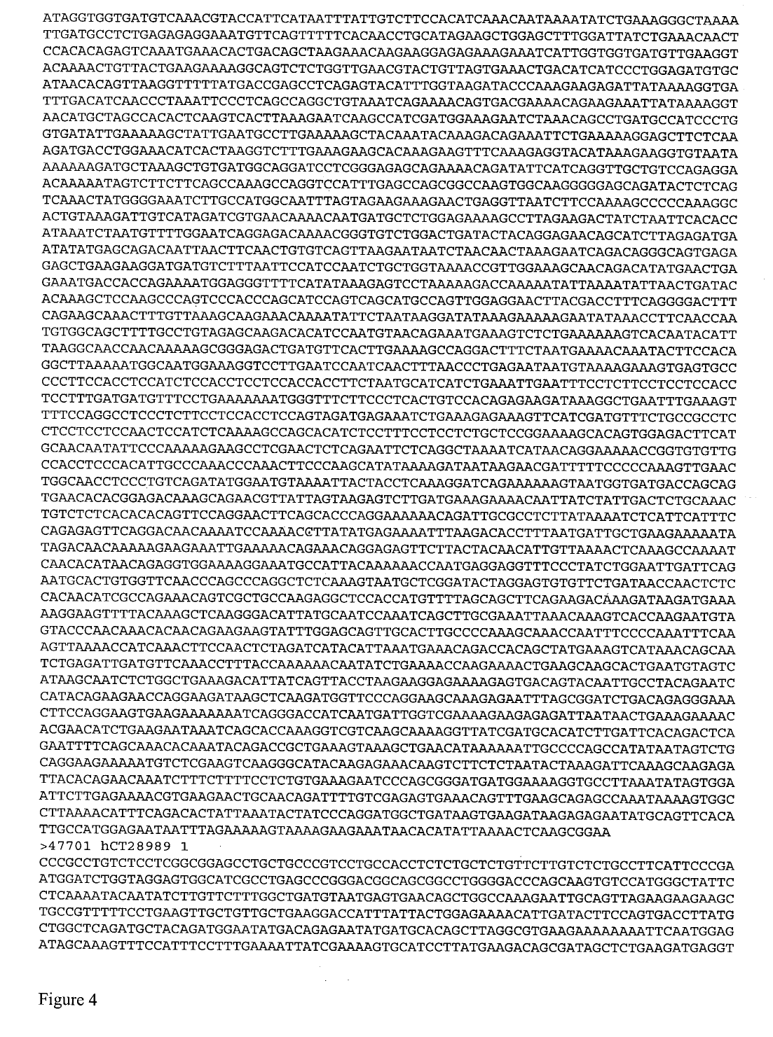

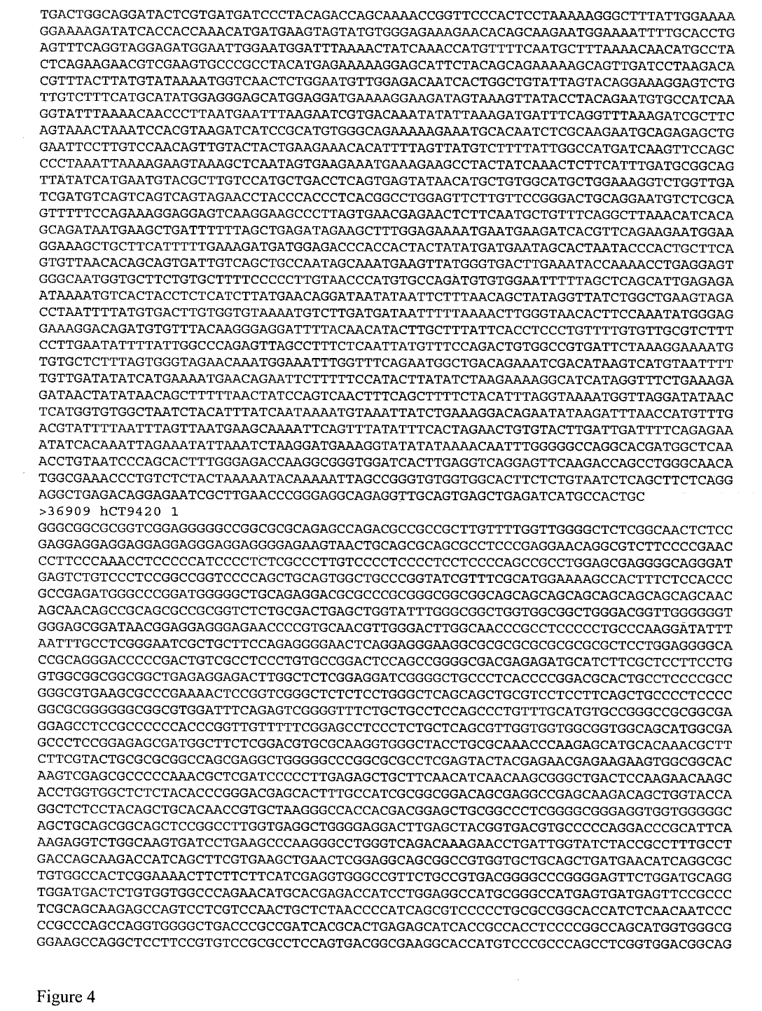

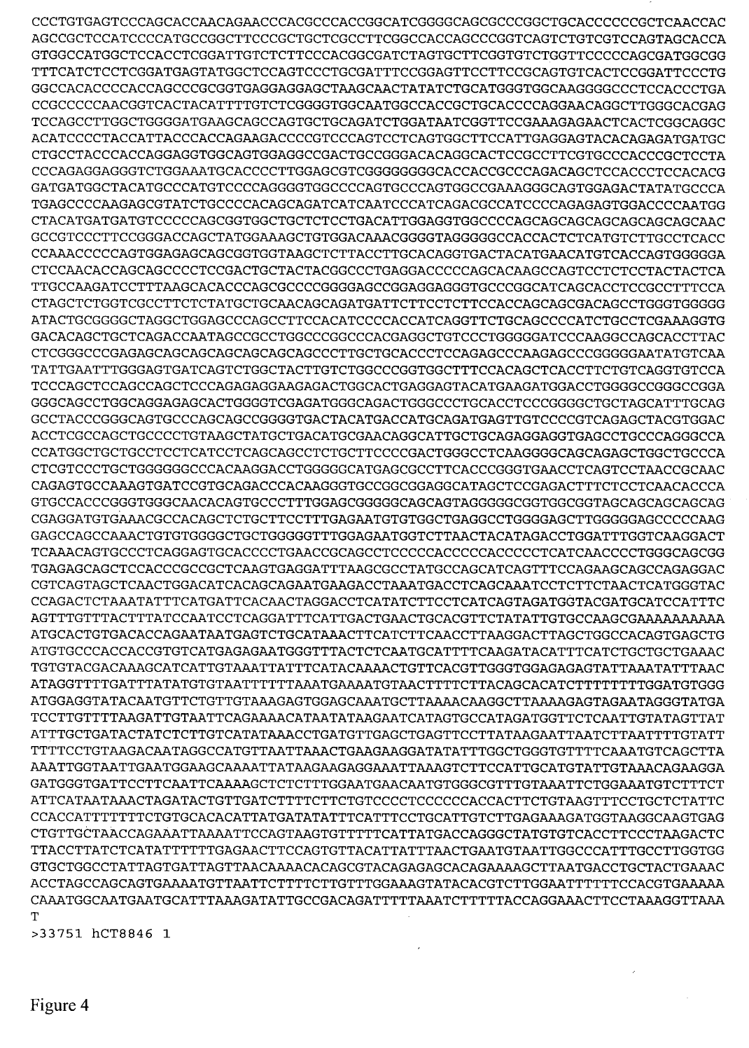

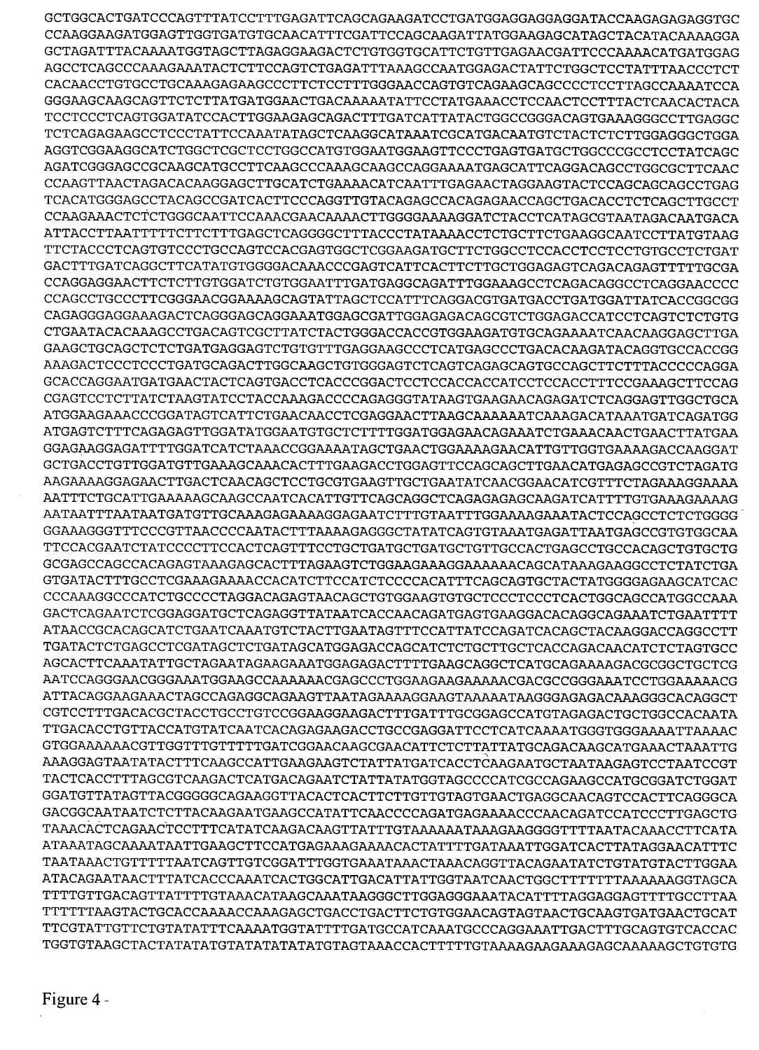

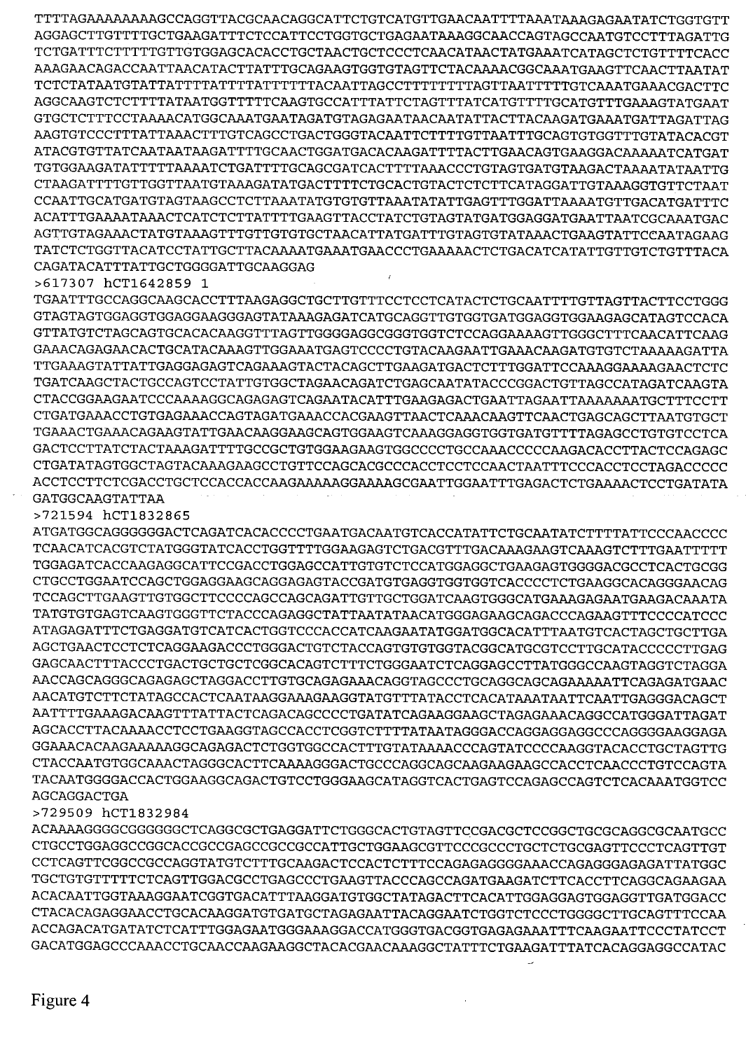

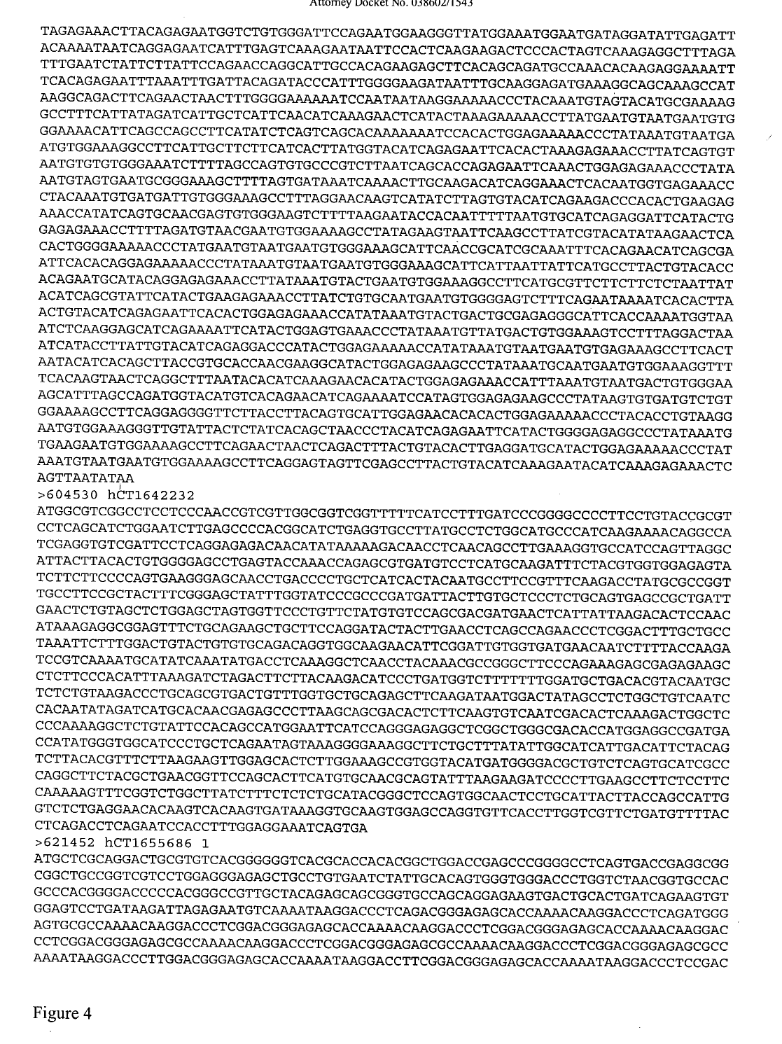

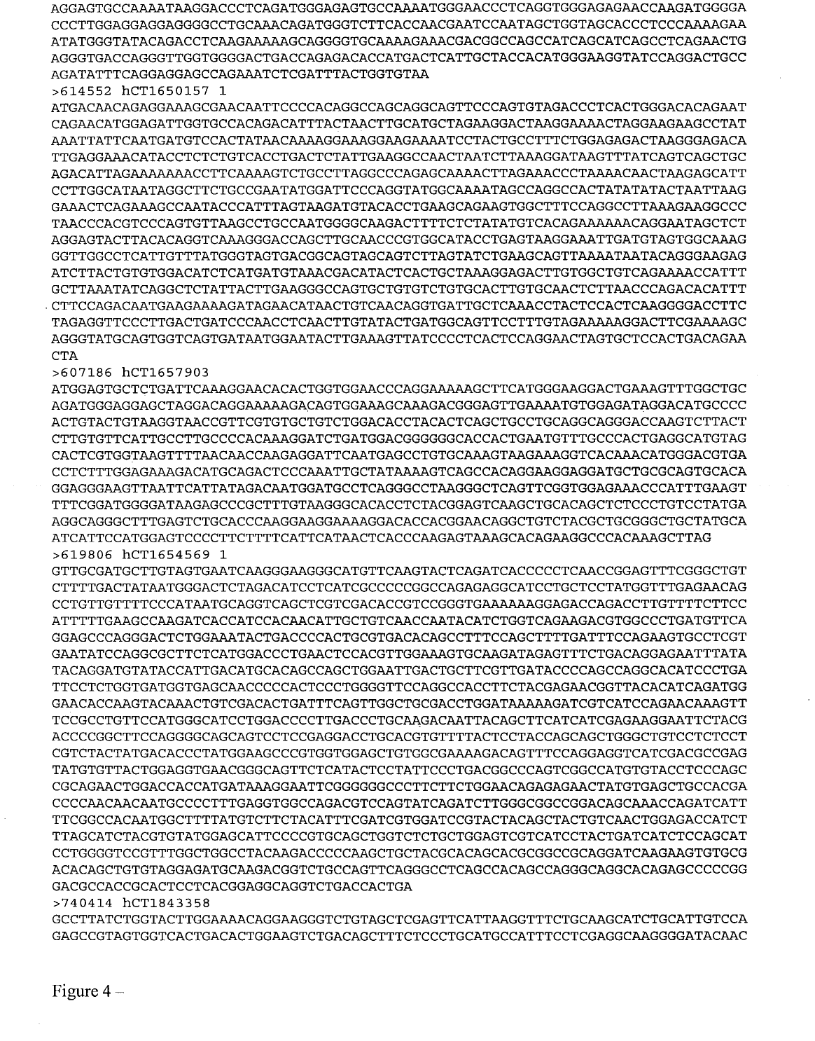

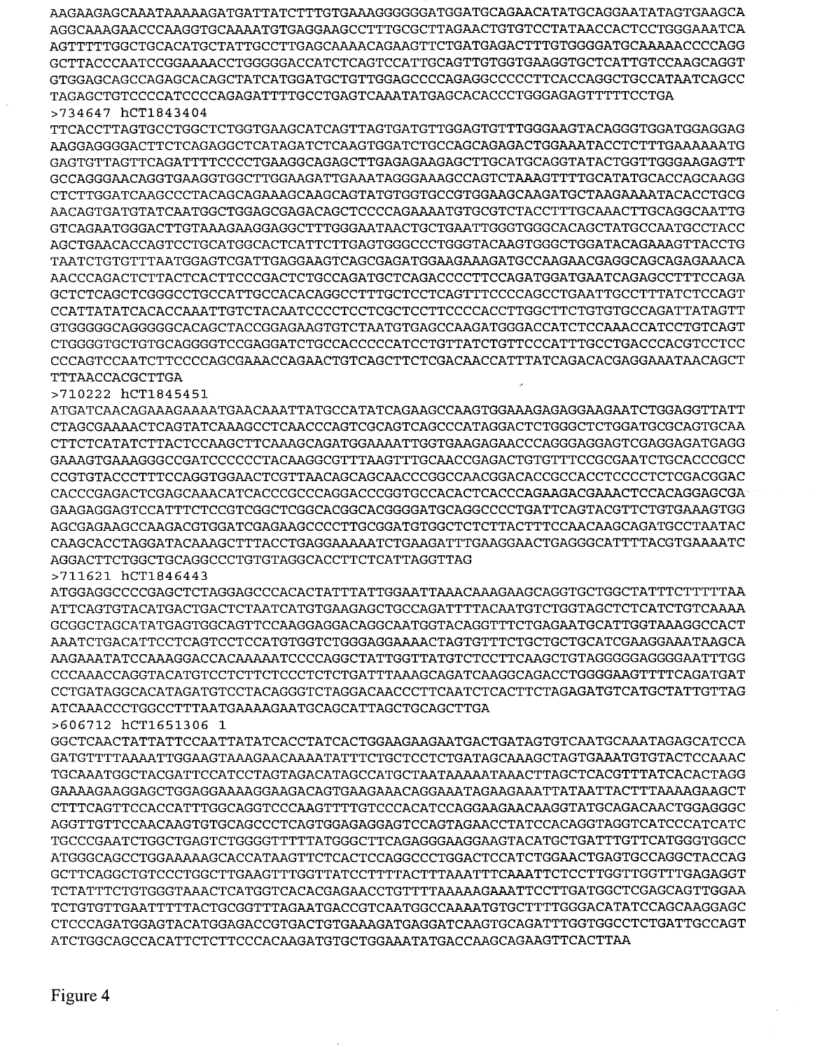

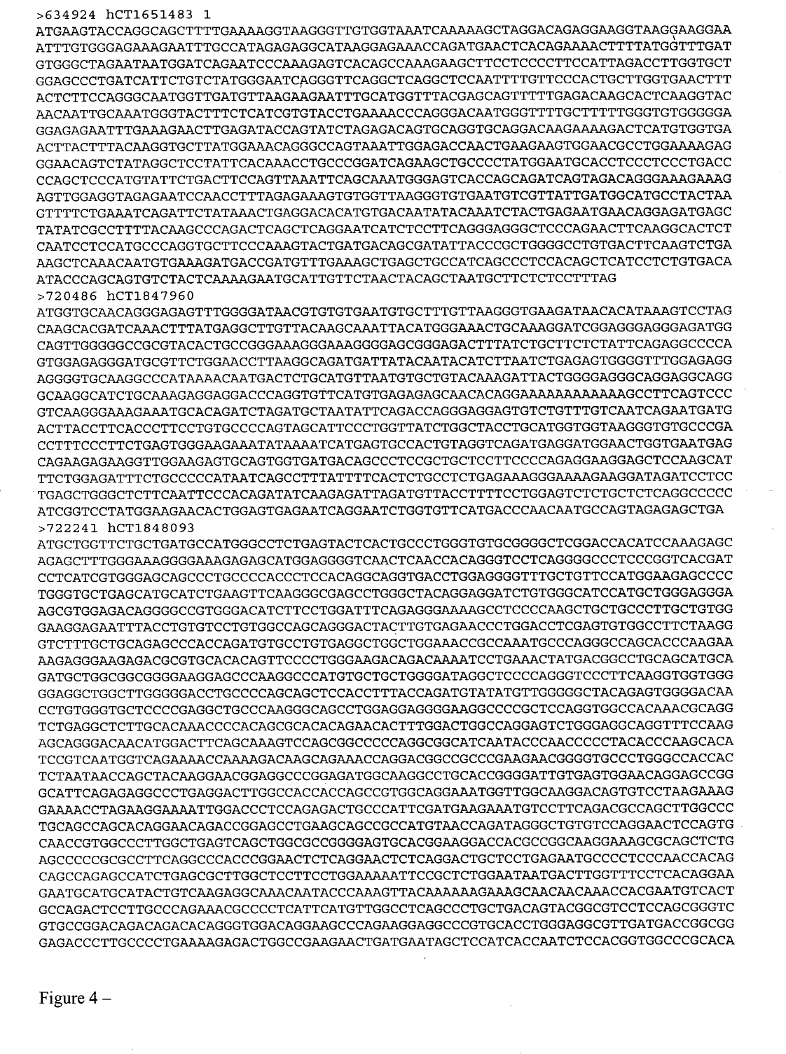

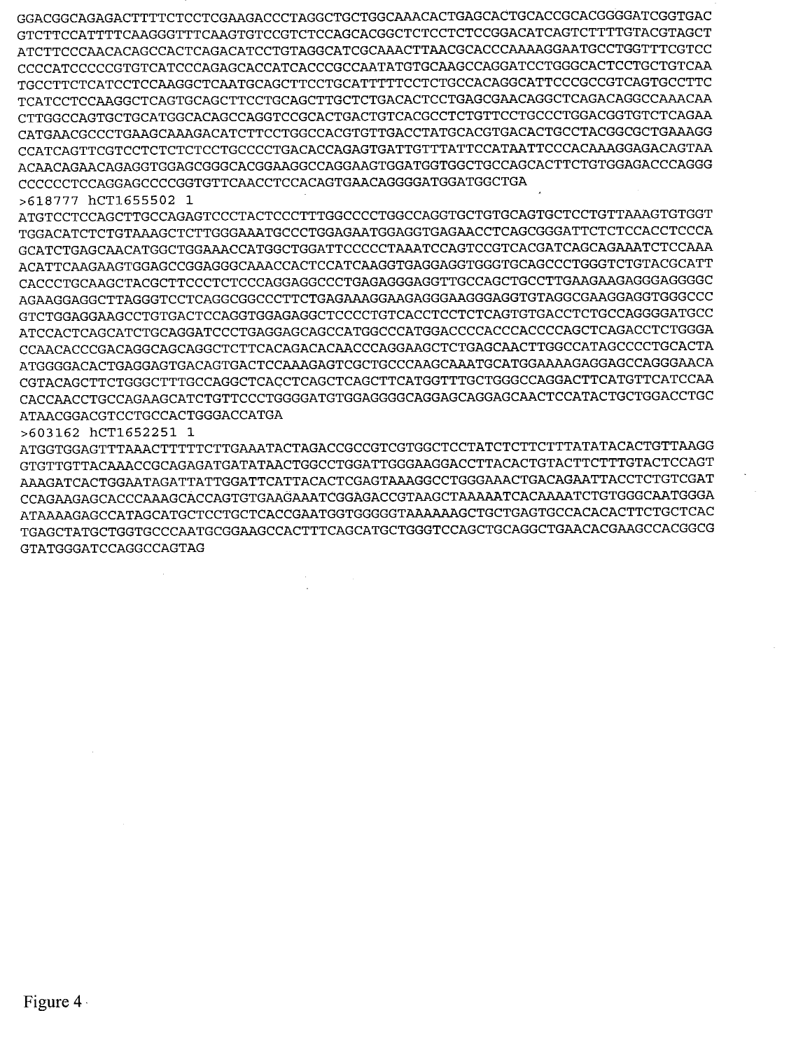

Using the novel CRISSP method for detecting remote polypeptide homologues, 87 novel kinases were identified. The nucleotide and amino acid sequences of these novel kinases, along with the predicted secondary structure, are shown in FIG. 2. The 87 novel genes are described below. ESTs identified in each novel gene are listed. Table 1 shows the list of tissues where gene-supporting ESTs were found for the listed genes. Table 2 shows the list of PFAM domains found in known protein kinases that have been identified in the novel kinases. Also listed for each gene is the presence and identity of any CRISSPs identified in the polypeptide sequence.

1) Gene 37589 (SEQ ID NO: 1, 88)—ESTs identified: 1501485.10, 1501910.1, 1501910.2, 1501485.7, 1501485.2, 1501485.4, 1501485.6, 1501485.1, gi|15307949, gi|4503764, gi|1518668, gi|6679816, gi|296587, gi|8252629, gi|433257, gi|398044, gi|182672. The CRISSP e_eK_hE_e_e_h_h_e_e was found between residues 105-214.

2) Gene 41528 (SEQ ID NO:2, 89) ESTs identified: 228543.20, 228543.19, 228543.13, 228543.6, 228543.3, 228543.7, 228543.1, gi|6807777, gi<18580889. The CRISSP e_e_eK_hE_e_e_e_h_h_e was found between residues 166-317.

3) Gene 41590 (SEQ ID NO: 3, 90)—The following ESTs were identified in this gene 200197.15, 200197.13, gi|7959260. The CRISSP e_e_eK_hE_h_e_e_h_h_e_e is found between residues 209-421.

4) Gene 604530 (SEQ ID NO:4, 91)—No ESTs identified. The CRISSP e_e_eK_hE_h_e_e_h_h_e_e is found between residues 565

5) Gene 618777 (SEQ ID NO: 5, 92) No ESTs were identified in this gene. The CRISSP e_e_eK_hE_e_e_e_h_h_e is found between residues 91-269

6) Gene 734647 (SEQ ID NO:6, 93)—No ESTs were identified in this gene. TheCRISSP e_e_eK_hE_e_e_h_h_h_e found between residues 1-174

7) Gene 740414 (SEQ ID NO: 7, 94)—No ESTs found. The CRISSP e_eK_hE_e_e_h_h_e_e is found between residues 36-170

8) Gene 740787 (SEQ ID NO: 8, 95)—The ESTs identified: 1383326.3, gi|16160477, gi|16579886, gi|394649, gi|1142969. CRISSP e_e_eK_hE_h_e_e_h_h_e_e found between residues 126-263.

9) Gene 740895 (SEQ ID NO:9, 96)—No ESTs identified. CRISSP e_e_eK_hE_e_e_h_h_h_e found between residues 229-377.

10) Gene 742686 (SEQ ID NO: 10, 97)—ESTs identified: 7698261.9, 7698261.8, 7698261.6, 7698261.5, 7698261.1, 7698261.2, 7698261.3, 7698261.4, 7698261.10, 7698261.11, gi|13623538, gi|12240052, gi|520832, gi|4507766.CRISSP e_eK_hE_e_e_h_h_e_e found between residues 939-1058.

11) Gene 201537 (SEQ ID NO:11, 98)—ESTs identified: 331470.6, 331470.41, 331470.40, 331470.33, gi|3043707, gi|7023098, gi|14039966, gi|7022865.CRISSP e_e_eK_hE_h_e_e_h_h_e_e found between residues 70-380.

12) Gene 33751 (SEQ ID NO: 12, 99)ESTs identitifed: 978380.20, 978380.16, 978380.19, 978380.5, 978380.1, gi|18553845, gi|10437958, gi|18490635.CRISSP e_e_eK_hE_e_e_e_h_h_e found between residues 88-283.

13) Gene 33795 (SEQ ID NO:13, 100)—ESTs identified: gi|13443025, gi|7022064, gi|10436582, 979146.2, 979146.4, 979146.6.CRISSP e_eK_hE_e_e_h_h_e_e found between residues 321-433.

14) Gene 33890—(SEQ ID NO:14, 101)—ESTs identified: 428010.1, gi|1507821.CRISSP e_e_eK_hE_e_e_h_h_h_e found between residues 801-955

15) Gene 34119 (SEQ ID NO:15, 102)—ESTs identified: 395034.24, 395034.27, 395034.19, 395034.16, 395034.11, 3405258CA2, 395034.1, gi|12276065, gi|13278845, gi|13518227,gi|10241725, gi|9049351, gi|13278914, gi|10434788, gi|14573194,gi|12382295.CRISSP e_eK_hE_e_e_h_h_e_e found between residues 321-460.

16) Gene 35501 (SEQ ID NO:16, 103)—ESTs identified: gi|190266, gi|178151, gi|337423, gi|190166, 474444.1, 474444.2, 474444.12, 474444.11, 474444.10.CRISSP e_e_e_eK_hE_e_e_h_h h_e found between residues 302-525.

17) Gene 35582 (SEQ ID NO:17, 104)—ESTs identified: 7693911.1, 7693911.2, 7693911.3, 7693911.4, 3050391CA2, gi|186377, gi|4504682, gi|511808, gi|511810, gi|511812, gi|511814, gi|559053.CRISSP e_e_eK_hE_e_e_h_h_h_e found between residues 2-205.

18) Gene 35689—(SEQ ID NO:18, 105) EST identified: 338865.2.CRISSP e_e_eK_hE_e_e_h_h_h_e found between residues 149-316.

19) Gene 36832 (SEQ ID NO:19,106)—ESTs identified: gi|15⁶²⁵⁵⁸⁵, gi|18042852, 1003888.10, 1003888.13, 1003888.6, 1003888.5, 1003888.7, 1003888.8, 1003888.9, 1003888.1, 1003888.2, 1003888.4, 1003888.17, 1003888.16, 1003888.19, 1003888.15, gi|15193508, gi|14042075, gi|16550110, gi|10241721, gi|16799068.CRISSP e_e_eK_hE_e_e_h_h_h_e found between residues 383-584.

20) Gene 36909 (SEQ ID NO:20, 107)—ESTs identified: gi|386256, gi|5031804, gi|14730385, 1134820.3, 1134820.14.CRISSP e_e_eK_hE_e_e_e_h_h_e found between residues 57-242.

21) Gene 38149—(SEQ ID NO:21, 108)—257949.1, gi|1932793, gi|12667419, gi|12667417, gi|12667415, gi|12667413, gi|2667411, gi|12667409, gi|16554448.CRISSP e_eK_hE_e_e_h_h_e_e found between residues 807-969.

22) Gene 38327—(SEQ ID NO: 22, 109)—ESTs identified: gi|397142, 349900.1, 349900.2.CRISSP e_e_eK_hE_e_e_h_h_h_e found between residues 320-435.

23) Gene 38609 (SEQ ID NO 23, 110)—ESTs identified: 375431.1, 332125.13, gi|18676525, gi|5420189.CRISSP e_e_eK_hE_e_e_e_h_h_e found between residues 37-196.

24) Gene 38645 (SEQ ID NO:24, 111)—ESTs identified: gi|20127568, gi|12803596, gi|7020886, 267667.20, 267667.19, 267667.14, 267667.9, 267667.7, 267667.1.CRISSP e_e_eK_hE_h_e_e_h_h_e_e found between residues 60-214.

25) Gene 39227 (SEQ ID NO:25, 112)—ESTs identified: gi|19913411, gi|19913409, gi|13509193,gi|13509191, gi|14714137, 1502262.2, 1502262.4, 1502262.6, 1502262.8, 2261821CA2, gi|15990477, gi|19577289, 1502262.19, 1502262.16, 1502262.18, 1502262.14, 1502262.11, 1502262.12, 1502262.13.CRISSP e_e_eK_hE_e_e_e_h_h_e found between residues 353-468.

26) Gene 40497 (SEQ ID NO:26, 113)—ESTs identified: 345496.3, gi|10336524, gi|984266, gi|9257259, gi|1314286.CRISSP e_e_eK_hE_e_e_e_h_h_e found between residues 1154-1359.

27) Gene 40893 (SEQ ID NO:27, 114)—ESTs identified: 228575.18, 228575.8, 228575.7, 228575.5, gi|495869, gi|443760, gi|522330, gi|310082, gi|495867.CRISSP e_eK_hE_e_e_h_h_e_e found between residues 1009-1162.

28) Gene 40980 (SEQ ID NO:28, 115)—ESTs identified: 7761828.3, 7761828.8, 7761828.1, 7761828.11, gi|3043701, gi|16193738.CRISSP e_e_eK_hE_h_e_e_h_h_e_e found between residues 258-486.

29) Gene 40994 (SEQ ID NO:29, 116)—ESTs identified: 233660.8, 233660.6, 7771270.1, 233660.28, 233660.27, 233660.25, 233660.20, gi|8923652, gi|12652938, gi|14041988, gi|12001943, gi|15451274, gi|7021005, gi|17389516, gi|18676499, gi|10440077.CRISSP e_e_eK_hE_e_e_e_e_h_he found between residues 334-537.

30) Gene 41437 (SEQ ID NO:30, 117) ESTs identified: 7771332.6, 7771332.9, 7771332.1, 7771332.2, 7771332.4, 7771332.16, 7771332.14, 7771332.10, gi|1136429, gi|14043414, gi|9956021.CRISSP e_e_eK_hE_e_e_e_h_h_e found between residues 51-175.

31) Gene 41509 (SEQ ID NO:31, 118)—ESTs identified: 981142.3, 899894.5, 899894.4, 899894.1, 899894.2, gi|19116192, gi|16550228, gi|12698086, gi|17390164, gi|17390162, gi|10433418, gi|16549998, gi|16550749.CRISSP e_e_eK_hE_e_e_h_h_h_e found between residues 1131-1307.

32) Gene 41822 (SEQ ID NO:32, 119)—ESTs identified: gi|561857, gi|3282238, gi|598226, gi|508481, gi|511228, 345252.10, 345252.12, 345252.19, 345252.31, 345252.30, 345252.32, 345252.1, 345252.3, 345252.7, 345252.8, 345252.9.CRISSP e_e_eK_hE_e_e_h_h_h_e found between residues 1470-1624.

33) Gene 41963 (SEQ ID NO:33, 120)—ESTs identified: 054241.14, 054241.10, 054241.9, gi|4753777, gi|6174884, gi|6631097.CRISSP e_e_eK_hE_e_e_e_h_h_e found between residues 513-657.

34) Gene 42024 (SEQ ID NO:34, 121)—ESTs identified: 1384029.4, 1384029.3, gi|10437738, gi|10438249, gi|13477218, gi|12232414.CRISSP e_e_eK_hE_e_e_e_h_h_e found between residues 90-219.

35) Gene 42804 (SEQ ID NO:35, 122)—ESTs identified: 1501991.16, gi|12803398, gi|10437981, 1501991.8, 1501991.6, 2768614CA2, 1501991.20, 1501991.19, gi|11527206, gi|4929638, gi|15277878, gi|19923797, gi|10439290, gi|10437646.CRISSP e_e_e_eK_hE_e_e_e_h_h_e found between residues 104-300.

36) Gene 42916 (SEQ ID NO:36, 123)—ESTs identified: gi|13325103, gi|10439191, gi|13386485, gi|19525701, gi|6807775, 196674.31, 196674.29, 196674.22.CRISSP e_e_eK_hE_e_e_e_h_h_e found between residues 5-133.

37) Gene 43542 (SEQ ID NO:37, 124)—ESTs identified: 1390118.5, 1390118.6, 1390118.2, gi|13111990, gi|19923811, gi|7020411, gi|17530000, gi|13569485.CRISSP e_eK_hE_e_e_h_h_e_e_e found between residues 42-163.

38) Gene 45900 (SEQ ID NO:38, 125)—ESTs identified: 202868.3, 202868.7, 202868.8, gi|17391413.CRISSP e_e_eK_hE_e_e_e_h_h_e found between residues 55-182.

39) Gene 46144 (SEQ ID JNO:39, 126)—ESTs identified: 363388.4, 363388.7, 363388.3, gi|3925684, gi|5453889, gi|17225575, gi|14713608, gi|14710878.CRISSP e_e_eK_hE_e_e_e_h_h_e found between residues 16-111.

40) Gene 47182 (SEQ ID NO:40, 127)—No ESTs identified.CRISSP e_e_e_eK_hE_e_e_h_h_h_e found between residues 164-316.

41) Gene 47225 (SEQ ID NO:41, 128)—ESTs identified: 340229.7, 340229.10.CRISSP e_e_eK_hE_e_e_h_h_h_e found between residues 1203-1363.

42) Gene 47701 (SEQ ID NO:42,129)—ESTs identified: 1095150.21, 1095150.22, 1095150.23, 1095150.5, 1095150.19, 1095150.13, 1095150.10, 1095150.11, gi|4507298, gi|16552923, gi|2338557, gi|16163883.CRISSP e_eK_hE_e_e_h_h_e_e found between residues 326-440.

43) Gene 48279 (SEQ ID NO:43, 130)—ESTs identified: 131713.26, 131713.20, 131713.23, 131713.18, 131713.11, 2150905CA2, 131713.40, 131713.41, 131713.38, 131713.37, 131713.34, gi|4885564, gi|4680312, gi|2243145, gi|16552135, gi|2318124.CRISSP e_e_eK_hE_h_e_e_h_h_e_e found between residues 93-276.

44) Gene 49476 (SEQ ID NO:44, 131)—ESTs identified: 234102.58, 234102.51, 234102.65, gi|15929608, gi|15929241.CRISSP e_eK_hE_e_e_h_h_e_e found between residues 194-314.

45) Gene 49799 (SEQ ID NO:45, 132)—ESTs identified: 334025.2, gi|927595, gi|6681258.CRISSP e_e_eK_hE_e_e_e_h_h_e found between residues 33-164.

46) Gene 49904 (SEQ ID NO:46, 133)—ESTs identified: 252800.5, 252800.28, 252800.27, 252800.23, 252800.18, 252800.17, 252800.16, 252800.15, 252800.12.CRISSP e_e_eK_hE_e_e_h_h_h_e found between residues 919-1175.

47) Gene 49964 (SEQ ID NO:47, 134)—ESTs identified: 036272.4, 036272.1, 036272.7, 036272.34, 036272.32, 036272.33, 036272.30, 036272.28, 036272.27, 036272.20, 036272.21, gi|13376863, gi|13277577, gi|15929211.CRISSP e_e_eK_hE_e_e_h_h_h_e found between residues 70-246.

48) Gene 50258 (SEQ ID NO:48, 135)—ESTs identified: gi|1848276, 411304.9, 411304.18, 411304.16, 411304.13, 411304.15, gi|16445439, gi|18583472.CRISSP e_e_e_eK_hE_e_e_h_h_h_e found between residues 131-303.

49) Gene 50347 (SEQ ID NO:49, 136)—ESTs identified: gi|6005823, gi|7022202, gi|4760646, gi|10433116, gi|12803432, gi|7020948, gi|10435082, 7771096.3, 7771096.6, 7771096.8, 7771096.1, 7771096.2, 7771096.11.CRISSP e_eK_hE_e_e_h_h_e_e found between residues 341-427.

50) Gene 51386 (SEQ ID NO:50, 137)—ESTs identified: gi|7662221, gi|3327103, 399359.16, 399359.17, 399359.18, 399359.19, 399359.12, 399359.5, 399359.4, 399359.2.CRISSP e_eK_hE_e_e_h_h_e_e found between residues 805-951.

51) Gene 51500 (SEQ ID NO:51, 138)—449173.24, 449173.31, 449173.32, gi|14714433,gi|1841746, 449173.12, 449173.9, gi|5730026, gi|12653852, gi|17512262, gi|189499.CRISSP e_eK_hE_e_e_h_h_e_e found between residues 82-242.

52) Gene 51594 (SEQ ID NO:52, 139)—ESTs identified: 331689.17, 331689.34, 331689.33, 331689.1, gi|608024, gi|4502092, gi|12052939, gi|1167995, gi|10947055.CRISSP e_eK_hE_e_e_h_h_e_e found between residues 2834-3059.

53) Gene 51975 (SEQ ID NO:53, 140)—No ESTs identified. CRISSP e_e_eK_hE_e_e_h_h_h_e found between residues 65-193.

54) Gene 603162 (SEQ ID NO:54, 141)—No ESTs identified.CRISSP e_e_eK_hE_e_e_e_h_h_e found between residues 17-152.

55) Gene 605203 (SEQ ID NO:55, 142)—No ESTs identified.CRISSP e_e_eK_hE_e_e_e_h_h_e found between residues 16-150.

56) Gene 606712 (SEQ ID NO:56, 143)—NO ESTs identified.CRISSP e_e_eK_hE_h_e_e_h_h_e_e found between residues 223-309.

57) Gene 607186 (SEQ ID NO:57, 144)—No ESTs identified.CRISSP e_e_eK_hE_h_e_e_h_h_e e found between residues 37-166.

58) Gene 608163 (SEQ ID NO:58, 145)—ESTs identified: 345336.17, 345336.2, 345336.4, 345336.8, gi|2218062, gi|16741288, gi|4505996.CRISSP e_e_eK_hE_h e_e_h_h_e_e found between residues 309-509.

59) Gene 611800 (SEQ ID NO:59, 146)—No ESTs identified.CRISSP e_eK_hE_e_e_h_h_e_e found between residues 103-339.

60) Gene 614552 (SEQ ID NO:60, 147)—No ESTs identified.CRISSP e_eK_hE_e_e_h_h_e_e found between residues 199-331.

61) Gene 617307 (SEQ ID NO:61, 148)—No ESTs identified.CRISSP e_e_eK_hE_e_e_h_h_h_e found between residues 39-200.

62) Gene 619806 (SEQ ID NO:62, 149)—ESTs identified: 068879.1, gi|15208238, gi|15208234.CRISSP e_e_eK_hE_e_e_e_h_h_e found between residues 43-139.

63) Gene 619844 (SEQ ID NO:63, 150)—ESTs identified: 7688522.1, gi|17485353, gi|13359162.CRISSP e_e_eK_hE_e_e_h_h_h_e found between residues 16-172.

64) Gene 621452 (SEQ ID NO:64, 151)—No ESTs identified.CRISSP e_e_eK_hE_h_e_e_h_h_e_e found between residues 79-263.

65) Gene 623354 (SEQ ID NO:65, 152)—7671558.1, gi|14744117, gi|13603892, gi|14277688.CRISSP e_e_eK_hE_e_e_e_h_h_e found between residues 2591-2718.

66) Gene 633964 (SEQ ID NO:66, 153)—No ESTs identified.CRISSP e_e_eK_hE_e_e_h_h_h_e found between residues 170-318.

67) Gene 634924 (SEQ ID NO:67,154)—No ESTs identified.CRISSP e_e_eK_hE_e_e_h_h_h_e found between residues 272-396.CRISSP e_eK_hE_e_e_h_h_e_e found between residues 290-401.

68) Gene 638212 (SEQ ID NO:68, 155)—EST identified: gi|18576334.

69) Gene 639598 (SEQ ID NO: 69, 156)—No ESTs identified. CRISSP e_e_eK_hE_e_e_e_h_h_e found between residues 44-194.

70) Gene 639989 (SEQ ID NO:70, 157)—ESTs identified: 034873.1, 034873.3, 034873.7, 034873.8, 034873.9, gi|10433985.CRISSP e_e_eK_hE_e_e_e_h_h_e found between residues 119-255.

71) Gene 689869 (SEQ ID NO:71, 158)—ESTs identified: gi|6503195, gi|4502660, gi|17440653, gi|180108, gi|264768, gi|264766, 1454378.10, 1454378.11, 1454378.4, 1454378.1, 1454378.2, 3507129CA2, 1454378.13.CRISSP e_e_eK_hE_e_e_h_h_h_e found between residues 129-293.

72) Gene 691401 (SEQ ID NO:72, 159)—ESTs identififed: gi|11231086, 1449826.8, 1449826.3, 1449826.10, gi|7657696, gi|7381351, gi|14133196, gi|18958236, gi|11494025, gi|11494023, gi|1494021, gi|11494019.CRISSP e_e_e_eK_hE_e_e_e_h_h_e found between residues 739-853.

73) Gene 698561 (SEQ ID NO:73, 160)—ESTs identified: 261207.15, 261207.10, 261207.6, 261207.5, 261207.9, gi|10438788, gi|14250749, gi|10438190, gi|7020652, gi|15620828, gi|14424670, gi|6093224.CRISSP e_eK_hE_e_e_h_h_e_e found between residues 368-454.

74) Gene 699545 (SEQ ID NO:74, 161)—ESTs identified: 449201.32, 449201.28, gi|5107940, gi|2665741, gi|3378171, gi|4506482, gi|3063674, gi|4079830, gi|3243259, gi|6978945, gi|9957535, gi|9957533.CRISSP e_eK_hE_e_e_h_h_e_e found between residues 2111-2225.

75) Gene 699704 (SEQ ID NO:75, 162)—ESTs identified: gi|10438041, gi|2055294, gi|19263955, 027987.571, 7691714.28, 7691714.27, 7691714.25, 7691714.21, 7691714.23, 7691714.15.CRISSP e_eK_hE_e_e_h_h_e_e found between residues 57-193.

76) Gene 710222 (SEQ ID NO:76, 163)—No ESTs identified.CRISSP e_e_eK_hE_h_e_e_h_h_e_e found between residues 42-249.

77) Gene 711621 (SEQ ID NO:77, 164)—No ESTs identified.CRISSP e_e_eK_hE_e_e_h_h_h_e found between residues 56-199.

78) Gene 718872 (SEQ ID NO:78, 165)—EST identified: gi|17459669.CRISSP e_e_eK_hE_h_e_e_h_h_e_e found between residues 267-447.

79) Gene 720486 (SEQ ID NO:79, 166)—ESTs identified: gi|18546754, 204417.1, 204417.2.CRISSP e_e_eK_hE_h_e_e_h_h_e_e found between residues 84-276.

80) Gene 721594 (SEQ ID NO: 80, 167)—ESTs identified: 1103621.1.CRISSP e_e_eK_hE_e_e_e_h_h_e found between residues 159-269.

81) Gene 722241 (SEQ ID NO:81, 168)—ESTs identified: gi|18593417.CRISSP e_eK_hE_e_e_h_h_e_e found between residues 71-256.

82) Gene 728098 (SEQ ID NO:82, 169)—ESTs identified: 7678776.1, gi|17391481.CRISSP e_eK_hE_e_e_h h_e_e found between residues 1124-1242.

83) Gene 728857 (SEQ ID NO:83, 170)—ESTs identified; gi|15144272, gi|15823645, gi|15823643, gi|18158216, 019520.1, 207593.34, 207593.3, 246150.1, 207593.1, gi|10437701, gi|18314418.CRISSP e_e_eK_hE_h_e_e_h_h_e_e found between residues 243-474.

84) Gene 729509 (SEQ ID NO:84, 171)—ESTs identified: 7693045.1. CRISSP e_e_eK_hE_e_e_h_h_h_e found between residues 754-861.

85) Gene 730440 (SEQ ID NO:85, 172)—ESTs identified: 2760114CA2, 1830678CA2, 334401.5, 334401.4, 334401.1, 334401.16, 334401.17, 334401.19, gi|4519442, gi|6453817, gi|456695, gi|456697, gi|13279307.CRISSP e_e_eK_hE_e_e_e_h_h_e found between residues 204-353.

86) Gene 730817 (SEQ ID NO:86, 173)—EST identified: gi|17459669. CRISSP e_e_eK_hE_h_e_e_h_h_e_e found between residues 266-445.

87) Gene 734179 (SEQ ID NO:87, 174)—ESTs identified: gi|15421857, gi|6599296, gi|10434118, gi|10436600, gi|14043080.CRISSP e_e_eK_hE_e_e_e_h_h_e found between residues 402-540.

### Example 8

### Isolation of cDNAs Encoding Mammalian Protein Kinases

Materials and Methods

Identification of Novel Clones

Total RNAs are isolated using the Guanidine Salts/Phenol extraction protocol of Chomczynski and Sacchi (P. Chomczynski and N. Sacchi, Anal. Biochem. 162, 156 (1987)) from primary human tumors, normal and tumor cell lines, normal human tissues, and sorted human hematopoietic cells. These RNAs are used to generate single-stranded cDNA using the Superscript Preamplification System (GIBCO BRL, Gaithersburg, Md.; Gerard, GF et al. (1989), FOCUS 11, 66) under conditions recommended by the manufacturer. A typical reaction uses 10 μg total RNA with 1.5 μg oligo(dT)₁₂₋₁₈ in a reaction volume of 60 μL. The product is treated with RNaseH and diluted to 100 μL with H₂O. For subsequent PCR amplification, 1-4 μL of this sscDNA is used in each reaction.

Degenerate oligonucleotides are synthesized on an Applied Biosystems 3948 DNA synthesizer using established phosphoramidite chemistry, precipitated with ethanol and used unpurified for PCR. These primers are derived from the sense and antisense strands of conserved motifs within the catalytic domain of several protein kinases. Degenerate nucleotide residue designations are: N=A, C, G, or T; R=A or G; Y=C or T; H=A, C or T not G; D=A, G or T not C; S=C or G; and W=A or T.

PCR reactions are performed using degenerate primers applied to multiple single-stranded cDNAs. The primers are added at a final concentration of 5 μM each to a mixture containing 10 mM Tris HCl, pH 8.3, 50 mM KCl, 1.5 mM MgCl₂, 200 μM each deoxynucleoside triphosphate, 0.001% gelatin, 1.5 U AmpliTaq DNA Polymerase (Perkin-Elmer/Cetus), and 1-4 μL cDNA. Following 3 min denaturation at 95° C., the cycling conditions are 94° C. for 30 s, 50° C. for 1 min, and 72° C. for 1 min 45 s for 35 cycles % PCR fragments migrating between 300-350 bp are isolated from 2% agarose gels using the GeneClean Kit (Bio101), and T-A cloned into the pCR11 vector (Invitrogen Corp. U.S.A.) according to the manufacturer's protocol.

Colonies are selected for mini plasmid DNA-preparations using Qiagen columns and the plasmid DNA is sequenced using a cycle sequencing dye-terminator kit with AmpliTaq DNA Polymerase, FS (ABI, Foster City, Calif.). Sequencing reaction products are run on an ABI Prism 377 DNA Sequencer, and analyzed using the BLAST alignment algorithm (Altschul, S. F. et al., J. Mol. Biol. 215: 403-10).

Additional PCR strategies are employed to connect various PCR fragments or ESTs using exact or near exact oligonucleotide primers. PCR conditions are as described above except the annealing temperatures are calculated for each oligo pair using the formula: Tm=4(G+C)+2(A+T).

Isolation of cDNA Clones:

Human cDNA libraries are probed with PCR or EST fragments corresponding to kinase-related genes. Probes are ³²P-labeled by random priming and used at 2×10⁶ cpm/mL following standard techniques for library screening. Pre-hybridization (3 h) and hybridization (overnight) are conducted at 42° C. in 5×SSC, 5× Denhart's solution, 2.5% dextran sulfate, 50 mM Na₂PO₄/NaHPO₄, pH 7.0, 50% formamide with 100 mg/mL denatured salmon sperm DNA. Stringent washes are performed at 65° C. in 0.1×SSC and 0.1% SDS. DNA sequencing is carried out on both strands using a cycle sequencing dye-terminator kit with AmpliTaq DNA Polymerase, FS (ABI, Foster City, Calif.). Sequencing reaction products are run on an ABI Prism 377 DNA Sequencer.

### Example 9

### Expression Analysis of Mammalian Protein Kinases

Materials and Methods

Northern Blot Analysis

Northern blots are prepared by running 10 μg total RNA isolated from 60 human tumor cell lines (such as HOP-92, EKVX, NCI-H23, NCI-H226, NCI-H322M, NCI-H460, NCI-H522, A549, HOP-62, OVCAR-3, OVCAR-4, OVCAR-5, OVCAR-8, IGROV1, SK-OV-3, SNB-19, SNB-75, U251, SF-268, SF-295, SF-539, CCRF-CEM, K-562, MOLT-4, HL-60, RPMI 8226, SR, DU-145, PC-3, HT-29, HCC-2998, HCT-116, SW620, Colo 205, HTC15, KM-12, UO-31, SN12C, A498, CaKil, RXF-393, ACHN, 786-0, TK-10, LOX IMVI, Malme-3M, SK-MEL-2, SK-MEL-5, SK-MEL-28, UACC-62, UACC-257, M14, MCF-7, MCF-7/ADR RES, Hs578T, MDA-MB-231, MDA-MB-435, MDA-N, BT-549, T47D), from human adult tissues (such as thymus, lung, duodenum, colon, testis, brain, cerebellum, cortex, salivary gland, liver, pancreas, kidney, spleen, stomach, uterus, prostate, skeletal muscle, placenta, mammary gland, bladder, lymph node, adipose tissue), and 2 human fetal normal tissues (fetal liver, fetal brain), on a denaturing formaldehyde 1.2% agarose gel and transferring to nylon membranes.

Filters are hybridized with random primed [α³²p]dCTP-labeled probes synthesized from the inserts of several of the kinase genes. Hybridization is performed at 42° C. overnight in 6×SSC, 0.1% SDS, 1× Denhardt's solution, 100 μg/mL denatured herring sperm DNA with 1-2×10⁶ cpm/mL of ³²P-labeled DNA probes. The filters are washed in 0.1×SSC/0.1% SDS, 65° C., and exposed on a Molecular Dynamics phosphorimager.

Quantitative PCR Analysis

RNA is isolated from a variety of normal human tissues and cell lines. Single stranded cDNA is synthesized from 10 μg of each RNA as described above using the Superscript Preamplification System (GibcoBRL). These single strand templates are then used in a 25 cycle PCR reaction with primers specific to each clone. Reaction products are electrophoresed on 2% agarose gels, stained with ethidium bromide and photographed on a UV light box. The relative intensity of the STK-specific bands were estimated for each sample.

DNA Array Based Expression Analysis

Plasmid DNA array blots are prepared by loading 0.5 μg denatured plasmid for each kinase on a nylon membrane. The [γ³² P]dCTP labeled single stranded DNA probes are synthesized from the total RNA isolated from several human immune tissue sources or tumor cells (such as thymus, dendrocytes, mast cells, monocytes, B cells (primary, Jurkat, RPMI8226, SR), T cells (CD8/CD4+, TH1, TH2, CEM, MOLT4), K562 (megakaryocytes). Hybridization is performed at 42° C. for 16 hours in 6×SSC, 0.1% SDS, 1× Denhardt's solution, 100 μg/mL denatured herring sperm DNA with 10⁶ cpm/mL of [γ³²P]dCTP labeled single stranded probe. The filters are washed in 0.1×SSC/0.1% SDS, 65° C., and exposed for quantitative analysis on a Molecular Dynamics phosphorimager.

### Example 10

### Protein Kinase Gene Expression

Vector Construction

Materials and Methods

Expression Vector Construction

Expression constructs are generated for some of the human cDNAs including: a) full-length clones in a pcDNA expression vector; b) a GST-fusion construct containing the catalytic domain of the novel kinase fused to the C-terminal end of a GST expression cassette; and c) a full-length clone containing a Lys to Ala (K to A) mutation at the predicted ATP binding site within the kinase domain, inserted in the PcDNA vector.

The “K to A” mutants of the kinase might function as dominant negative constructs.

### Example 11

### Generation of Specific Immunoreagents to Protein Kinases

Materials and Methods

Specific immunoreagents are raised in rabbits against KLH- or MAP-conjugated synthetic peptides corresponding to isolated kinase polypeptides. C-terminal peptides were conjugated to KLH with glutaraldehyde, leaving a free C-terminus. Internal peptides were MAP-conjugated with a blocked N-terminus. Additional immunoreagents can also be generated by immunizing rabbits with the bacterially expressed GST-fusion proteins containing the cytoplasmic domains of each novel PTK or STK.

The various immune sera are first tested for reactivity and selectivity to recombinant protein, prior to testing for endogenous sources.

Western Blots

Proteins in SDS PAGE are transferred to immobilon membrane. The washing buffer is PBST (standard phosphate-buffered saline pH 7.4+0.1% Triton X-100). Blocking and antibody incubation buffer is PBST+5% milk. Antibody dilutions varied from 1:1000 to 1:2000.

### Example 12

### Recombinant Expression and Biological Assays for Protein Kinases

Materials and Methods

Transient Expression of Kinases in Mammalian Cells

The pcDNA expression plasmids (10 μg DNA/100 mm plate) containing the kinase constructs are introduced into 293 cells with lipofectamine (Gibco BRL). After 72 hours, the cells are harvested in 0.5 mL solubilization buffer (20 mM HEPES, pH 7.35, 150 mM NaCl, 10% glycerol, 1% Triton X-100, 1.5 mM MgCl₂, 1 mM EGTA, 2 mM phenylmethylsulfonyl fluoride, 1 μg/mL aprotinin). Sample aliquots are resolved by SDS polyacrylamide gel electrophoresis (PAGE) on 6% acrylamide/0.5% bis-acrylamide gels and electrophoretically transferred to nitrocellulose. Non-specific binding is blocked by preincubating blots in Blotto (phosphate buffered saline containing 5% w/v non-fat dried milk and 0.2% v/v nonidet P-40 (Sigma)), and recombinant protein was detected using the various anti-peptide or anti-GST-fusion specific antisera.

In Vitro Kinase Assays

Three days after transfection with the kinase expression constructs, a 10 cm plate of 293 cells is washed with PBS and solubilized on ice with 2 mL PBSTDS containing phosphatase inhibitors (10 mM NaHPO₄, pH 7.25, 150 mM NaCl, 1% Triton X-100, 0.5% deoxycholate, 0.1% SDS, 0.2% sodium azide, 1 mM NaF, 1 mM EGTA, 4 mM sodium orthovanadate, 1% aprotinin, 5 μg/mL leupeptin). Cell debris was removed by centrifugation (12000×g, 15 min, 4° C.) and the lysate was precleared by two successive incubations with 50 μL of a 1:1 slurry of protein A sepharose for 1 hour each. One-half mL of the cleared supernatant was reacted with 10 μL of protein A purified kinase-specific antisera (generated from the GST fusion protein or antipeptide antisera) plus 50 μL of a 1:1 slurry of protein A-sepharose for 2 hr at 4° C. The beads were then washed 2 times in PBSTDS, and 2 times in HNTG (20 mM HEPES, pH 7.5/150 mM NaCl, 0,1% Triton X-100, 10% glycerol).

The immunopurified kinases on sepharose beads are resuspended in 20 μL HNTG plus 30 mM MgCl₂, 10 mM MnCl₂, and 20 μCi [α³²p]ATP (3000 Ci/mmol). The kinase reactions are run for 30 min at room temperature, and stopped by addition of HNTG supplemented with 50 mM EDTA. The samples are washed 6 times in HNTG, boiled 5 min in SDS sample buffer and analyzed by 6% SDS-PAGE followed by autoradiography. Phosphoamino acid analysis is performed by standard 2D methods on ³²P-labeled bands excised from the SDS-PAGE gel.

Similar assays are performed on bacterially expressed GST-fusion constructs of the kinases.

### Example 13a

### Chromosomal Localization of Protein Kinases

Materials and Methods

Several sources are used to find information about the chromosomal localization of each of the genes described in this patent. First, cytogenetic map locations of these contigs are found in the title or text of their Genbank record, or by inspection through the NCBI human genome map viewer (http://www.ncbi.nlm.nih.gov/cgi-bin/Entrez/hum_srch?).

Alternatively, the accession number of a genomic contig (identified by BLAST against NRNA) is used to query the Entrez Genome Browser (http://www.ncbi.nlm.nih.gov/PMGifs/Genomes/MapViewerHelp.html), and the cytogenetic localization is read from the NCBI data. A thorough search of available literature for the cytogenetic region is also made using Medline (http://www.ncbi.nlm.nih.gov/PubMed/medline.html). References for association of the mapped sites with chromosomal amplifications found in human cancer can be found in: Knuutila, et al., Am J Pathol, 1998, 152:1107-1123.

Alternatively, the accession number for the nucleic acid sequence is used to query the Unigene database. The site containing the Unigene search engine is: http://www.ncbi.nlm.nih.gov/UniGene/Hs.Home.html. Information on map position within the Unigene database is imported from several sources, including the Online Mendelian Inheritance in Man (OMIM, http://www.ncbi.nlm.nih.gov/Omim/searchomim.html), The Genome Database (http://gdb.infobiogen.fr/gdb/simpleSearch.html), and the Whitehead Institute human physical map (http://carbon.wi.mit.edu:8000/cgi-bin/contig/sts_info?database=release).

Once a cytogenetic region has been identified by one of these approaches, disease association can be established by searching OMIM with the cytogenetic location. OMIM maintains a searchable catalog of cytogenetic map locations organized by disease. A thorough search of available literature for the cytogenetic region is also made using Medline (http://www.ncbi.nlm.nih.gov/PubMed/medline.html). As noted above, references for association of the mapped sites with chromosomal abnormalities found in human cancer can be found in: Knuutila, et al., Am J Pathol, 1998, 152:1107-1123.

The chromosomal positions can be cross-checked with the Online Mendelian Inheritance in Man database (OMIM, http://www.ncbi.nlm.nih.gov/htbin-post/Omim), which tracks genetic information for many human diseases, including cancer. References for association of the mapped sites with chromosomal abnormalities found in human cancer can be found in: Knuutila, et al., Am J Pathol, 1998, 152:1107-1123. A third source of information on mapped positions was searching published literature (at NCBI, http://www.ncbi.nlm.nih.gov/entrez/query.fcgi) for documented association of the mapped position with human disease.

### Example 13b

### Candidate Single Nucleotide Polymorphisms (SNPS)

Materials and Methods

The most common variations in human DNA are single nucleotide polymorphisms (SNPs), which occur approximately once every 100 to 300 bases. Because SNPs are expected to facilitate large-scale association genetics studies, there has recently been great interest in SNP discovery and detection. Candidate SNPs for the genes in this application aare identified by blastn searching the nucleic acid sequences against the public database of sequences containing documented SNPs (dbSNP: sequence files were downloaded from ftp://ncbi.nlm.nih.gov/SNP/human/rs-fasta/ and ftp://ncbi.nlm.nih.gov/SNP/human/ss-fasta/ and used to create a blast database). dbSNP accession numbers for the SNP-containing sequences are given. SNPs are also identified by comparing several databases of expressed genes (dbEST, NRNA) and genomic sequence (i.e., NRNA) for single basepair mismatches. The code below is standard for representing DNA sequence when describing SNPs:

| | | |
|---|---|---|
| G | = | Guanosine |
| A | = | Adenosine |
| T | = | Thymidine |
| C | = | Cytidine |
| R | = | C or A, puRine |
| Y | = | C or T, pYrimidine |
| K | = | G or T, Keto |
| W | = | A or T, Weak (2 H-bonds) |
| S | = | C or C, Strong (3 H-bonds) |
| M | = | A or C, aMino |
| B | = | C, C or T (i.e., not A) |
| D | = | A, C or T (i.e., not C) |
| H | = | A, C or T (i.e., not C) |
| V | = | A, C or C (i.e., not T) |
| N | = | A, C, C or T, aNy |
| X | = | A, C, C or T |

| | |
|---|---|
| complementary | G A T C R Y W S K M B V D H N X |
| DNA | +−+−+−+−+−+−+−+−+−+−+−+−+−+−+−+−+ |
| strands | C T A G Y R S W M K V B H D N X |

For example, if two versions of a gene exist, one with a “C” at a given position, and a second one with a “T: at the same position, then that position is represented as a Y, which means C or T. In table 2, for SGK002, the SNP column says “1165=R”, which means that at position 1165, a polymorphism exists, with that position sometimes containing a G and sometimes an A (R represents A or G). SNPs may be important in identifying heritable traits associated with a gene.

### Example 14

### Demonstration of Gene Amplification by Southern Blotting

Materials and Methods

Nylon membranes are purchased from Boehringer Mannheim. Denaturing solution contains 0.4 M NaOH and 0.6 M NaCl. Neutralization solution contains 0.5 M Tris-HCL, pH 7.5 and 1.5 M NaCl. Hybridization solution contains 50% formamide, 6×SSPE, 2.5× Denhardt's solution, 0.2 mg/mL denatured salmon DNA, 0.1 mg/mL yeast tRNA, and 0.2% sodium dodecyl sulfate. Restriction enzymes are purchased from Boehringer Mannheim. Radiolabeled probes are prepared using the Prime-it II kit by Stratagene. The beta actin DNA fragment used for a probe template is purchased from Clontech.

Genomic DNA is isolated from a variety of tumor cell lines (such as MCF-7, MDA-MB-231, Calu-6, A549, HCT-15, HT-29, Colo 205, LS-180, DLD-1, HCT-116, PC3, CAPAN-2, MIA-PaCa-2, PANC-1, AsPc-1, BxPC-3, OVCAR-3, SKOV3, SW 626 and PA-1, and from two normal cell lines.

A 10 μg aliquot of each genomic DNA sample is digested with EcoR I restriction enzyme and a separate 10 μg sample is digested with Hind III restriction enzyme. The restriction-digested DNA samples are loaded onto a 0.7% agarose gel and, following electrophoretic separation, the DNA is capillary-transferred to a nylon membrane by standard methods (Sambrook, J. et al (1989) Molecular Cloning: A Laboratory Manual, Cold Spring Harbor Laboratory).

### Example 15

### Detection of Protein-Protein Interaction Through Phage Display

Materials and Methods

Phage display provides a method for isolating molecular interactions based on affinity for a desired bait. cDNA fragments cloned as fusions to phage coat proteins are displayed on the surface of the phage. Phage(s) interacting with a bait are enriched by affinity purification and the insert DNA from individual clones is analyzed.

T7 Phage Display Libraries

All libraries were constructed in the T7Select1-1b vector (Novagen) according to the manufacturer's directions.

Bait Presentation

Protein domains to be used as baits are generated as C-terminal fusions to GST and expressed in E. coli. Peptides are chemically synthesized and biotinylated at the N-terminus using a long chain spacer biotin reagent.

Selection

Aliquots of refreshed libraries (10¹⁰-10¹² pfu) supplemented with PanMix and a cocktail of E. coli inhibitors (Sigma P-8465) are incubated for 1-2 hrs at room temperature with the immobilized baits. Unbound phage is extensively washed (at least 4 times) with wash buffer.

After 3-4 rounds of selection, bound phage is eluted in 100 μL of 1% SDS and plated on agarose plates to obtain single plaques.

Identification of Insert DNAs

Individual plaques are picked into 25 μL of 10 mM EDTA and the phage is disrupted by heating at 70° C. for 10 min. 2 μL of the disrupted phage are added to 50 μL PCR reaction mix. The insert DNA is amplified by 35 rounds of thermal cycling (94° C., 50 sec; 50° C., Imin; 72° C., 1 min).

Composition of Buffer

10×PanMix

5% Triton X-100

10% non-fat dry milk (Carnation)

10 mM EGTA

250 mM NaF

250 μg/mL Heparin (sigma)

250 μg/mL sheared, boiled salmon sperm DNA (sigma)

0.05% Na azide

Prepared in PBS

Wash Buffer

PBS supplemented with:

```
0.5% NP-40
25 μl g/mL heparin
PCR reaction mix
  1.0 mL 10x PCR buffer (Perkin-Elmer, with 15
mM Mg)
  0.2 mL each dNTPs (10 mM stock)
  0.1 mL T7UP primer (15 pmol/μL)
GGAGCTCTCGTATTCCAGTC
  0.1 mL T7DN primer (15 pmol/μL)
AACCCCTCAAGACCCGTTTAG
  0.2 mL 25 mM MgCl₂ or MgSO₄ to compensate for
EDTA
Q.S. to 10 mL with distilled water
Add 1 unit of Taq polymerase per 50 μL reaction
LIBRARY: T7 Select1-H441
```

### Example 16

### HUV-EC-C Assay

The following protocol may also be used to measure a compound's activity against any endogenous kinase which is naturally expressed by HUV-EC cells.

DAY 0

1. Wash and trypsinize HUV-EC-C cells (human umbilical vein endothelial cells, (American Type Culture Collection; catalogue no. 1730 CRL). Wash with Dulbecco's phosphate-buffered saline (D-PBS; obtained from Gibcp BRL; catalogue no. 14190-029) 2 times at about 1 ml/10 cm² of tissue culture flask. Trypsinize with 0.05% trypsin-EDTA in non-enzymatic cell dissociation solution (Sigma Chemical Company; catalogue no. C-1544). The 0.05% trypsin was made by diluting 0.25% trypsin/1 mM EDTA (Gibco; catalogue no. 25200-049) in the cell dissociation solution. Trypsinize with about 1 ml/25-30 cm² of tissue culture flask for about 5 minutes at 37° C. After cells have detached from the flask, add an equal volume of assay medium and transfer to a 50 ml sterile centrifuge tube (Fisher Scientific; catalogue no. 05-539-6).

2. Wash the cells with about 35 ml assay medium in the 50 ml sterile centrifuge tube by adding the assay medium, centrifuge for 10 minutes at approximately 200 g, aspirate the supernatant, and resuspend with 35 ml D-PBS. Repeat the wash two more times with D-PBS, resuspend the cells in about 1 ml assay medium/15 cm² of tissue culture flask. Assay medium consists of F12K medium (Gibco BRL; catalogue no. 21127-014)+0.5% heat-inactivated fetal bovine serum. Count the cells with a Coulter Counter™ Coulter Electronics, Inc.) and add assay medium to the cells to obtain a concentration of 0.8-1.0×105 cells/ml.

3. Add cells to 96-well flat-bottom plates at 100 μl/well or 0.8-1.0×10⁴ cells/well; incubate ˜24 h at 37° C., 5% CO2.

DAY 1

1. Make up two-fold drug titrations in separate 96-well plates, generally 50 μM on down to 0 μM. Use the same assay medium as mentioned in day 0, step 2, above. Titrations are made by adding 90 μl/well of drug at 200 μM (4×the final well concentration) to the top well of a particular plate column. Since the stock drug concentration is usually 20 mM in DMSO, the 200 μM drug concentration contains 2% DMSO.

Therefore, diluent made up to 2% DMSO in assay medium (F12K+0.5% fetal bovine serum) is used as diluent for the drug titrations in order to dilute the drug but keep the DMSO concentration constant. Add this diluent to the remaining wells in the column at 60 μl/well. Take 60 μl from the 120 μl of 200 μM drug dilution in the top well of the column and mix with the 60 μl in the second well of the column. Take 60 μl from this well and mix with the 60 μl in the third well of the column, and so on until two-fold titrations are completed. When the next-to-the-last well is mixed, take 60 μl of the 120 μl in this well and discard it. Leave the last well with 60 μl of DMSO/media diluent as a non-drug-containing control. Make 9 columns of titrated drug, enough for triplicate wells each for 1) VEGF (obtained from Pepro Tech Inc., catalogue no. 100-200, 2) endothelial cell growth factor (ECGF) (also known as acidic fibroblast growth factor, or aFGF) (obtained from Boehringer Mannheim Biochemica, catalogue no. 1439 600); or, 3) human PDGF B/B (1276-956, Boehringer Mannheim, Germany) and assay media control. ECGF comes as a preparation with sodium heparin.

2. Transfer 50 μl/well of the drug dilutions to the 96-well assay plates containing the 0.8-1.0×10⁴ cells/100 μl/well of the HUV-EC-C cells from day 0 and incubate ˜2 h at 37° C., 5% Co₂.

3. In triplicate, add 50 μl/well of 80 μg/ml VEGF, 20 ng/ml ECGF, or media control to each drug condition. As with the drugs, the growth factor concentrations are 4×the desired final concentration. Use the assay media from day 0, step 2, to make the concentrations of growth factors. Incubate approximately 24 hours at 37° C., 5% CO₂. Each well will have 50 μl drug dilution, 50 μl growth factor or media, and 100 μl cells, =200 μl/well total. Thus the 4×concentrations of drugs and growth factors become 1×once everything has been added to the wells.

DAY 2

1. Add ³H-thymidine (Amersham; catalogue no. TRK-686) at 1 μCi/well (10 μl/well of 100 μCi/ml solution made up in RPMI media+10% heat-inactivated fetal bovine serum) and incubate ˜24 h at 37° C., 5% CO₂. Note: ³H-thymidine is made up in RPMI media because all of the other applications for which we use the ³H-thymidine involve experiments done in RPMI. The media difference at this step is probably not significant. RPMI was obtained from Gibco BRL, catalogue no. 11875-051.

DAY 3

1. Freeze plates overnight at −20° C.

DAY 4

1. Thaw plates and harvest with a 96-well plate harvester (Tomtec Harvester 96^{(R)}) onto filter mats (Wallac; catalogue no. 1205-401); read counts on a Wallac Betaplate™ liquid scintillation counter.

### CONCLUSION

One skilled in the art would readily appreciate that the present invention is well adapted to carry out the objects and obtain the ends and advantages mentioned, as well as those inherent therein. The molecular complexes and the methods, procedures, treatments, molecules, specific compounds described herein are presently representative of preferred embodiments, are exemplary, and are not intended as limitations on the scope of the invention. It will be readily apparent to one skilled in the art that varying substitutions and modifications may be made to the invention disclosed herein without departing from the scope and spirit of the invention.

All patents and publications mentioned in the specification are indicative of the levels of those skilled in the art to which the invention pertains. All patents and publications are herein incorporated by reference to the same extent as if each individual publication was specifically and individually indicated to be incorporated by reference.

The invention illustratively described herein suitably may be practiced in the absence of any element or elements, limitation or limitations that are not specifically disclosed herein. Thus, for example, in each instance herein any of the terms “comprising,” “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

In addition, where features or aspects of the invention are described in terms of Markush groups, those skilled in the art will recognize that the invention is also thereby described in terms of any individual member or subgroup of members of the Markush group. For example, if X is described as selected from the group consisting of bromine, chlorine, and iodine, claims for X being bromine and claims for X being bromine and chlorine are fully described.

In view of the degeneracy of the genetic code, other combinations of nucleic acids also encode the claimed peptides and proteins of the invention. For example, all four nucleic acid sequences GCT, GCC, GCA, and GCG encode the amino acid alanine. Therefore, if for an amino acid there exists an average of three codons, a polypeptide of 100 amino acids in length will, on average, be encoded by 3100, or 5×1047, nucleic acid sequences. Thus, a nucleic acid sequence can be modified to form a second nucleic acid sequence, encoding the same polypeptide as encoded by the first nucleic acid sequences, using routine procedures and without undue experimentation. Thus, all possible nucleic acids that encode the claimed peptides and proteins are also fully described herein, as if all were written out in full taking into account the codon usage, especially that preferred in humans. Furthermore, changes in the amino acid sequences of polypeptides, or in the corresponding nucleic acid sequence encoding such polypeptide, may be designed or selected to take place in an area of the sequence where the significant activity of the polypeptide remains unchanged. For example, an amino acid change may take place within a β-turn, away from the active site of the polypeptide. Also changes such as deletions (e.g. removal of a segment of the polypeptide, or in the corresponding nucleic acid sequence encoding such polypeptide, which does not affect the active site) and additions (e.g. addition of more amino acids to the polypeptide sequence without affecting the function of the active site, such as the formation of GST-fusion proteins, or additions in the corresponding nucleic acid sequence encoding such polypeptide without affecting the function of the active site) are also within the scope of the present invention. Such changes to the polypeptides can be performed by those with ordinary skill in the art using routine procedures and without undue experimentation. Thus, all possible nucleic and/or amino acid sequences that can readily be determined not to affect a significant activity of the peptide or protein of the invention are also fully described herein.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein. Other embodiments are within the following claims.

### REFERENCES

Russell R B, Sasieni P D, Sternberg M J. (1998) Supersites within superfolds. Binding site similarity in the absence of homology. J. Mol. Biol. 2;282(4):903-18.

Russell R B, Saqi M A, Bates P A, Sayle R A, Sternberg M J. (1998) Recognition of analogous and homologous protein folds—assessment of prediction success and associated alignment accuracy using empirical substitution matrices. Protein Eng. 11(1):1-9.

Russell R B, Saqi M A, Sayle R A, Bates P A, Sternberg M J. (1997) Recognition of analogous and homologous protein folds: analysis of sequence and structure conservation. J. Mol. Biol. 269(3):423-39.

Grigoriev I V, Zhang C., Kim S H (2001) Sequence-based detection of distantly related proteins with the same fold. Protein Eng. 14(7):455-8

Grigoriev I V, Kim S H (1999) Detection of protein fold similarity based on correlation of amino acid properties. Proc Natl Acad Sci USA. 96(25):14318-23.

Fischer D, Eisenberg D. (1996) Protein fold recognition using sequence-derived predictions. Protein Sci. 5(5):947-55.

Elofsson A, Fischer D, Rice D W, Le Grand S M, Eisenberg D. (1996) A study of combined structure/sequence profiles. Fold Des. 1(6):451-61.

Fischer D, Rice D, Bowie J U, Eisenberg D. (1996) Assigning amino acid sequences to 3-dimensional protein folds. FASEB J. 10(1):126-36.

Xu H, Aurora R, Rose G D, White R H. (1999) Identifying two ancient enzymes in Archaea using predicted secondary structure alignment. Nat Struct Biol. 6(8):750-4.

Przytycka T, Aurora R, Rose G D. (1999) A protein taxonomy based on secondary structure. Nat Struct Biol. 6(7):672-82.

Aurora R, Rose G D. (1998) Seeking an ancient enzyme in Methanococcus jannaschii using ORF, a program based on predicted secondary structure comparisons. Proc Natl Acad Sci USA. 95(6):2818-23.

Kasuya A, Thornton J M. (1999) Three-dimensional structure analysis of PROSITE patterns. J. Mol. Biol. 286(5):1673-1691.

Wallace A C, Borkakoti N, Thornton J M. (1997) TESS: a geometric hashing algorithm for deriving 3D coordinate templates for searching structural databases. Application to enzyme active sites. Protein Sci. 6(11):2308-2323.

Wallace A C, Laskowski R A, Thornton J M (1996) Derivation of 3D coordinate templates for searching structural databases: application to Ser-His-Asp catalytic triads in the serine proteinases and lipases. Protein Sci. 5(6):1001-1013.

H. M. Berman, J. Westbrook, Z. Feng, G. Gilliland, T. N. Bhat, H. Weissig, I. N. Shindyalov, P. E. Bourne (2000) The Protein Data Bank. Nucleic Acids Research, 28 pp. 235-242

F. C. Bernstein, T. F. Koetzle, G. J. Williams, E. E. Meyer Jr, M. D. Brice, J. R. Rodgers, O. Kennard, T. Shimanouchi, M. Tasumi (1977) The Protein Data Bank: a computer-based archival file for macromolecular structures. J. Mol. Biol. 112 pp. 535

Mctigue, M. A., Wickersham, J. A., Pinko, C., Showalter, R. E., Parast, C. V., Tempczyk-Russell, A., Gehring, M. R., Mroczkowski, B., Kan, C. C., Villafranca, J. E., Appelt, K. (1999) Crystal Structure of the Kinase Domain of Human Vascular Endothelial Growth Factor Receptor 2: A Key Enzyme in Angiogenesis. Structure (London) 7 pp. 319

Mohammadi, M., McMahon, G., Sun, L., Tang, C., Hirth, P., Yeh, B. K., Hubbard, S. R., Schlessinger, J. (1997) Structures of the tyrosine kinase domain of fibroblast growth factor receptor in complex with inhibitors. Science 276 pp. 955

Niefind, K., Guerra, B., Pinna, L. A., Issinger, O. G., Schomburg, D. (1998) Crystal structure of the catalytic subunit of protein kinase CK2 from Zea mays at 2.1 A resolution. EMBO J. 17 pp. 2451

Shi L, Potts M, Kennelly P J (1998) The serine, threonine, and/or tyrosine-specific protein kinases and protein phosphatases of prokaryotic organisms: a family portrait. FEMS Microbiology Reviews 22 pp 229\hat{ }253

Altschul S F, Madden T L, Schaffer A A, Zhang J, Zhang Z, Miller W, Lipman D J (1997) Gapped BLAST and PSI-BLAST: a new generation of protein database search programs. Nucleic Acids Res. 25(17):3389-402

Brenner S E, Koehl P, Levitt M (2000) The ASTRAL compendium for protein structure and sequence analysis. Nucleic Acids Res. 28(1):254-6

Leonard C J, Aravind L, Koonin E V (1998) Novel families of putative protein kinases in bacteria and archaea: evolution of the “eukaryotic” protein kinase superfamily. Genome Res. 8(10):1038-47

Sonnhammer E L, Eddy S R, Durbin R. (1997) Pfam: a comprehensive database of protein domain families based on seed alignments. Proteins. 28(3):405-20

TABLE 1
List of tissues where gene-supporting ESTs were found (Part 1)

| | |
|---|---|
| #639989: | teratocarcinoma (NT2) |
| #49964: | Cervix, carcinoma |
| | Skin, melanotic melanoma. |
| #41963: | fibroblast |
| #619806: | testis |
| #47701: | placenta, pool, LICR, EF |
| | kidney, cortex, mw/renal cell CA, 65M |
| | brain, allocortex/neocortex, cingulate, aw/CA, 55F, RP |
| | brain, hippocampus, aw/atherosclerosis, CHF, 81F |
| | liver/spleen, fetal, 20wM, NORM, WM |
| | lung, aw/Patau's, fetal, 20wM |
| | probable periph blood, eosinophils, asthma, M/F |
| | uterus tumor, CA, pool, LICR, EF |
| | testis, M, TIGR |
| | colon, mw/adenoCA, 55M, m/COLHTUT01, COLHTUS02 |
| | sm intestine, ileum, mw/carcinoid, 30F |
| | synovium, elbow, rheuA, 51F |
| | CML precursor line, K-562, 53F, t/PMA-96 hr |
| | Jurkat line, T-cell leukemia, M, Untx, 5C-FL, EF |
| | Jurkat line, T-cell leukemia, M, t/PMA |
| | Jurkat line, T-cell leukemia, M, t/PMA, Iono-1 hr, 5C-FL, EF |
| | T-lymphocytes, CD4+, pool, t/anti-CD28 antibodies |
| | UCB, derived dendritic cells, M, t/PMA, Iono-5 hr |
| | adipose, pericecal, aw/cecal tubular adenoma, 55F |
| | adrenal gland, 20M |
| | adrenal gland, epithelium, 16wM, TIGR |
| | adrenal tumor, pheochromocytoma, 57F |
| | bladder tumor line, CA/TC CA/papilloma, pool, MGC, EF |
| | bone tumor line, MG-63, osteoSar, 14M, untreated |
| | bone tumor line, osteoSAR, MGC, EF |
| | bone tumor, rib, mets osteoSAR, 16M |
| | bone, rib, aw/Paget-Schroetter, 57M |
| | bone, rib, aw/Patau's, fetal, 20wM |
| | brain tumor, anaplastic oligodendroglioma, pool, NORM, CGAP |
| | brain tumor, benign meningioma, 35F, 5RP |
| | brain tumor, frontal, astrocytoma, 17F |
| | brain tumor, frontal, meningioma, 50M |
| | brain tumor, frontal, mets hypernephroma, 58M |
| | brain tumor, frontal, oligoastrocytoma, 50F |
| | brain, aw/hypoplastic left heart, fetal, 23wM, NORM |
| | brain, aw/spinal muscular atrophy, 72dF, NORM, SIK |
| | brain, caudate nucleus, mw/CVA, 92M |
| | brain, caudate/putamen/nucleus accumbens, aw/CHF, 35M |
| | brain, cerebellum, Huntington's, mw/CVA, 57M |
| | brain, frontal, Huntington's, mw/CVA, 57M |
| | brain, globus pallidus/substantia innominata, aw/CHF, 35M |
| | brain, infant, 10wF, NORM, WM |
| | brain, midbrain, aw/CHF, 35M |
| | brain, mw/oligoastrocytoma, epilepsy, 26M |
| | brain, neurogenic tumor line, SK-N-MC, neuroepithelioma, 14F |
| | brain, parietal cortex, aw/CHF, 35M |
| | brain, temporal cortex, aw/aortic aneurysm, 45F |
| | breast tumor line, T-47D, ductal CA, 54F, Untx, NORM, EF |
| | breast tumor line, adenoCA, MGC, EF |
| | breast tumor, adenoCA, 45F, m/BRSTNOT09 |
| | breast tumor, high vascular density, CA, F |
| | breast tumor, lobular CA, 58F, m/BRSTNOT05 |
| | breast tumor, low vascular density, control, F |
| | breast, 46F |
| | breast, 56F |
| | breast, PF breast disease, 57F |
| | breast, PF changes, mw/adenoCA, 45F, m/BRSTTUT08 |
| | breast, mw/lobular CA, 58F, m/BRSTTUT03 |
| | breast, papillomatosis, mw/lobular CA, 59F, m/BRSTTUT22 |
| | bronchial, epithelial cells, 23M, t/20% smoke 20 hr |
| | bronchus, smooth muscle cells, 21M, untreated |
| | cartilage, OA |
| | cervical tumor line, HeLa S3, adenoCA, 31F, untreated, WM/WN |
| | cervical tumor line, HeLa, adenoCA, 31F, t/PMA, CHX 4 hr |
| | cervix, cervicitis, 35F |
| | clavicle, osteoblasts, 40M, untreated |
| | colon polyp, aw/adenoCA, tubulovillous adenoma, 40F |
| | colon tumor, CA, pool, LICR, EF |
| | colon tumor, adenoCA, NORM, 3′ CGAP |
| | colon tumor, cecum, adenoCA, 70F |
| | colon tumor, ileocecum, Burkitt lymphoma, 29F, m/COLANOT03 |
| | colon, 25M, WN |
| | colon, CUC, 69M |
| | colon, ascending, CUC, 25F |
| | colon, ascending, mw/Burkitt lymphoma, 29F, m/COLITUT02 |
| | colon, cecum, Crohn's, 31M |
| | colon, cecum, mw/Crohn's, 18F |
| | colon, descending, benign familial polyposis, 16M |
| | colon, epithelium, 13F |
| | colon, mw/adenoCA, aw/node mets, 60M, m/COLNTUT16 |
| | colon, sigmoid, mw/adenoCA, aw/node mets, 62M, m/COLNTUT03 |
| | colon, transverse, benign familial polyposis, 16M |
| | coronary artery, smooth muscle cells, 3M |
| | coronary artery, smooth muscle cells, 3M, t/TNF, IL-1 |
| | embryo, 8w, TIGR |
| | epididymis, M, TIGR |
| | esophagus, aw/adenoCA, 61M |
| | esophagus, mw/adenoCA, aw/node mets, 53M |
| | esophagus, mw/adenoCA, aw/node mets, 53M, 5RP |
| | eye, retina, 55M, NORM, WM |
| | fat, abdomen, aw/obesity, 52F |
| | fat, mesentery, aw/diverticulosis, diverticulitis, 71M |
| | fetus, 8-9w, pool, NORM, CGAP/WM/WN |
| | fibroblast line, GD23A, t/radiation 5 min |
| | fibroblasts, senescent, NORM, WM/WN |
| | gallbladder, 25F, TIGR |
| | gallbladder, cholecystitis, cholelithiasis, 21M |
| | gallbladder, cholecystitis, cholelithiasis, 53F |
| | ganglion, dorsal root, cervical, aw/lymphoma, 32M |
| | ganglion, dorsal root, thoracic, aw/lymphoma, 32M |
| | ganglion, dorsal root, thoracic/lumbar, aw/lymphoma, 32M |
| | germ cell tumor, pool, SUB, 3′ CGAP |
| | heart coronary artery endothelial cells, 3M, untreated, NORM |
| | heart, coronary artery, CAD, 46M |
| | heart, coronary artery, endothelial cells, 58M |
| | heart, hypoplastic left, fetal, 23wM |
| | heart, left atrium, 51F |
| | kidney epithelial transf embryo line, 293-EBNA, Untx |
| | kidney tumor line, CA/adenoCA/hypernephroma, pool, MGC, EF |
| | kidney, MGC, EF |
| | kidney, aw/anencephaly, fetal, 17wF |
| | kidney, aw/hypoplastic left heart, fetal, 23wM |
| | kidney, pool, NORM, 3′ CGAP |
| | kidney, pool, SUB, 3′ CGAP |
| | liver tumor line, C3A, Hepatob, 15M, Untx, NORM |
| | liver tumor, mets colon adenoCA, 51F |
| | liver, 49M |
| | liver, aw/Patau's syndrome, fetal, 20wM, 5RP |
| | liver, aw/Patau's, anencephaly, fetal, pool, lg cDNA |
| | liver, aw/Patau's, fetal, 20wM |
| | liver, fetal, M, 5RP |
| | lung tumor line, sm/lg cell CA/mucoepidermoid CA, pool, MGC, EF |
| | lung tumor, adenoCA, 53M, m/LUNGNOT28 |
| | lung tumor, neuroendocrine carcinoid, pool, NORM, 3′ CGAP |
| | lung tumor, squamous cell CA, pool, 3′, CGAP |
| | lung, 35F, 5RP |
| | lung, aw/anencephaly, fetal, 20wF |
| | lung, fetal, 19w, NORM, CGAP/WM/WN |
| | lung, mw/adenoCA, 43M |
| | lung, mw/adenoCA, aw/node, diaphragm mets, 63F |
| | lung, mw/mets osteoSAR, aw/pleura mets, 58M |
| | lung, pneumonitis, mw/squamous cell CA, 69M, m/LUNGTUT03 |
| | lung, right bronchus, nonasthmatic, 18-55M/F, pool |
| | lymph node tumor, mets squamous cell CA, aw/tongue CA, 5RP |
| | lymph node, B-lymphocytes, germinal center, pool, MGC, EF |
| | mammary, epithelial cells, 21F, untreated, NORM |
| | mesentery tumor, sigmoid, mets mixed-mullerian tumor, 61F |
| | microvascular, dermal, endothelial cells, 22F, t/VEGF, EF |
| | mixed tissue tumor, head/neck, CA, pool, LICR, EF |
| | mixed tissues, fetal lung, testis, B-cell, SUB, 3′ CGAP/WN |
| | mixed tissues, includes tumor, SUB, 3′ CGAP |
| | mixed tissues, includes tumor, pool, SUB, CGAP |
| | mixed tissues, myometrium, smooth muscle cells, 57M/41F, B |
| | mixed tumor, Wilm's/brain mets, pool, 3′ CGAP |
| | mixed, (melanocytes, uterus, fetal heart), SUB, CGAP/WM/WN |
| | monocyte line, U937, histiocytic lymphoma, 37M, untreated |
| | mouth tumor, CA in situ, m/ORALTMP01, CGAP |
| | muscle, calf, mw/gangrene, aw/atherosclerosis, 67M |
| | muscle, skeletal |
| | muscle, skeletal, leg, 19F, GEXP |
| | muscle, thigh, mw/lipoSAR, 58M, RP |
| | muscle, tibial, aw/thrombosis, 41F |
| | nose, nasal polyp, eosinophilia, aw/asthma, 78M |
| | ovary tumor, adenoCA, 58F |
| | ovary tumor, mixed types, pooled, F, 3′ CGAP |
| | ovary tumor, mucinous cystadenoCA, 43F, m/OVARNOT03 |
| | ovary tumor, seroanaplastic CA, 52F |
| | ovary, 49F, WM/WN |
| | ovary, aw/menorrhagia, 47F, 5RP |
| | pancreas, 29M |
| | pancreas, 8M, 5RP |
| | pancreas, fetal, 23wM |
| | pancreas, islet cells, WM |
| | pancreas, islet cells, pool |
| | pancreas, pancreatitis, mw/adenoCA, 65F, m/PANCTUT01 |
| | parathyroid tumor, adenoma, M/F, NORM, WM |
| | penis, corpus cavernosum, M |
| | periph blood, granulocytes, M/F, t/GM-CSF |
| | periph blood, macrophages, adher PBMC, M/F, MLR-24 hr |
| | periph blood, macrophages, adher PBMC, M/F, t/LPS |
| | periph blood, monocytes, 42F, t/IL-10, LPS |
| | periph blood, monocytes, 42F, t/antiIL-10, LPS |
| | periph blood, monocytes, 42F, t/antiIL-10, LPS, SUB |
| | periph blood, promonocyte line, THP-1, AML, stimulated |
| | pituitary, 16-70M/F, pool |
| | placenta, fetal, 8-9w, pool, NORM, CGAP/WM |
| | placenta, fetal, M, WM |
| | placenta, neonatal, F, NORM, WM |
| | probable astrocytes, M/F, untreated |
| | prostate tumor, adenoCA, 66M, m/PROSTMT02 |
| | prostate, 28M, NORM |
| | prostate, 32M, SUB, 3′ CGAP |
| | prostate, AH, mw/adenoCA, 60M, m/PROSTUT08 |
| | prostate, AH, mw/adenoCA, 68M |
| | prostate, BPH, mw/adenoCA, 70M, SUB |
| | prostate, epithelium, M, 3′ CGAP |
| | prostate, pool, M, LICR, EF |
| | renal vein, smooth muscle cells, 57M, Untx |
| | seminal vesicle, aw/adenoCA, 61M |
| | skin, breast, 26F |
| | skin, foreskin, melanocytes, M, NORM, WM/WN |
| | skin, tuberculoid/lepromatous leprosy, M/F, pool, AMP |
| | sm intestine, 31F, RP |
| | sm intestine, 8M |
| | sm intestine, duodenum, 16M |
| | sm intestine, fetal, M, RP |
| | sm intestine, ileum, aw/adenoCA cecum, node mets, 70F, 5RP |
| | sm intestine, ileum, mw/carcinoid, adenoCA, F, pool, lg cDNA |
| | soft tissue tumor, spinal schwannoma, 35M |
| | soft tissue, retroperitoneal/supraglottic, pool, AMP |
| | spinal cord, aw/renal failure, 71M, NORM |
| | spleen, Gaucher's, 22M |
| | spleen, fetal, WM |
| | spleen, fetal, aw/hypoplastic left heart, 23wM, pool, AMP |
| | stomach tumor, adenoCA, 52M, m/STOMNOT02 |
| | stomach tumor, adenoCA, poorly differentiated, 3′ CGAP |
| | stomach, aw/esophagus adenoCA, 23w-61M, pool, AMP |
| | stomach, fetal, 18wM |
| | stomach, gastritis, mw/adenoCA, node mets, 76M, RP |
| | stomach, mw/adenoCA, node mets, 52M, m/STOMTUT01 |
| | synovium, hip, rheuA, 68F |
| | synovium, wrist, rheuA, 56F |
| | testis, 10-61M, pool, lg cDNA |
| | testis, 16M |
| | testis, M, NORM, CGAP/WN |
| | testis, aw/cirrhosis, 37M |
| | testis, necrosis, 31M |
| | thymus, aw/patent ductus arteriosus, 3M |
| | thymus, aw/patent ductus arteriosus, 3M, 5RP |
| | thymus, fetal, M |
| | thyroid tumor, follicular adenoma, 17M |
| | thyroid tumor, papillary CA, 3′ CGAP |
| | thyroid, lymphocytic thyroiditis, mw/papillary CA, 13F |
| | tongue tumor, squamous cell CA, 36M |
| | tonsil, B-lymphocytes, germinal, aw/tonsillitis, NORM, CGAP |
| | tonsil, lymphoid hyperplasia, 6M |
| | umb cord blood, mononuclear cells |
| | umb cord blood, mononuclear cells, M/F, t/G-CSF, lg cDNA |
| | umbilical vein, endothelial cells, HUVEC, t/TNFa-48 hr, 5C-FL, EF |
| | umbilical vein, endothelial cells, pool, WM/WN |
| | uterus tumor, endometrial adenoCA, F, pooled, 3′ CGAP |
| | uterus tumor, leiomyoma, 34F |
| | uterus tumor, serous papillary CA, F, pooled, 3′ CGAP |
| | uterus, aw/cardiomyopathy, 59F |
| | uterus, aw/ovarian follicular cysts, 34F |
| | uterus, cervix tumor line, CA, pool, MGC, EF |
| | uterus, mw/leiomyoma, aw/colon adenoCA, 45F |
| | brain, aw/hypoplastic left heart, fetal, 23wM, 5RP |
| | brain, cerebellum, aw/COPD, left ventricular failure, 70M |
| | breast tumor, ductal adenoCA, 66F |
| | colon, normal/pseudopolyp, Crohn, 16, 26M, pool, lg cDNA |
| | eye, corneal fibroblasts primary line, 76, untreated |
| | heart, fetal, 8-10w, pool, BI |
| | heart, fetal, TIGR |
| | iliac artery, endothelial cells, F, control, untreated |
| | periph blood, T-lymphocytes, CD8+, 63M, untreated |
| | thymus, aw/anencephaly, fetal, 17wF |
| | testis |
| #721594: | brain tumor line, DU 145, mets prostate CA, 69M, Untx, 5C-FL, EF |
| | brain, cingulate, aw/MI, 85F, AMP/N |
| | brain, temporal cortex, aw/aortic aneurysm, 45F, RP |
| | ovary tumor, serous CA, mets colon CA, 44F, 5RP, EF |
| #48279: | adrenal tumor, pheochromocytoma, 52F, EF |
| | bone tumor/line, MG-63, osteoSAR/giant cell, M/F, pool, RP, EF |
| | breast, PF changes, mw/adenoCA, 55F, m/BRSTTUT01 |
| | breast, PF changes, mw/ductal adenoCA, 54F, m/BRSTTUT02 |
| | colon tumor, adenoCA, 60M, m/COLNNOT07/08/09/11 |
| | heart, aorta, 17F |
| | heart, right atrium, 51F |
| | liver tumor, hepatocellular CA, pool, CHGC, EF |
| | prostate tumor, adenoCA, 59M, SUB, m/PROSNOT19 |
| | teratoCA line, NT2, Untx, NORM |
| | teratoCA line, hNT2, untreated, WM/WN |
| | cervical tumor line, HeLa, adenoCA, 31F, t/PMA, CHX 4 hr |
| | adrenal gland, mw/pheochromocytoma, 43F, m/ADRETUT07 |
| | adrenal glands, aw/anencephaly, fetal, 16wF, lg cDNA |
| | brain, aw/spinal muscular atrophy, 72dF, NORM, SIK |
| | brain, cerebellum, Huntington's, mw/CVA, 57M, RP |
| | brain, cingulate, aw/MI, 85F, AMP/N |
| | brain, frontal, gliosis, mw/epilepsy, cerebral palsy, 27M |
| | brain, hippocampus, aw/CHF, 35M, NORM |
| | brain, infant, 10wF, NORM, WM |
| | brain, parietal cortex, aw/CHF, 35M, AMP/N |
| | brain, temporal cortex, aw/aortic aneurysm, 45F |
| | breast tumor, CA, pool, LICR, EF |
| | breast tumor, adenoCA, 46F, m/BRSTNOT17 |
| | breast, 35F |
| | breast, PF changes, mw/multifocal ductal CA in situ, 46F |
| | colon line, pool, LICR, EF |
| | colon, descending, benign familial polyposis, 16M |
| | eye, retina, 55M, NORM, WM |
| | ganglion, dorsal root, cervical, aw/lymphoma, 32M, NORM, EF |
| | heart, fetal, 19w, NORM, CGAP/WM/WN |
| | heart, left ventricle, Pompe's, 7mM, RP |
| | placenta, aw/hydrocephalus, fetal, 16w, RP |
| | prostate, mw/adenoCA, 65M |
| | spleen, Gaucher's, 22M |
| | T-lymphocytes, allogenic, 40-50M, untreated |
| | adrenal gland, 8M |
| | bladder tumor, TC CA, 60M, m/BLADNOT05 |
| | brain tumor, anaplastic oligodendroglioma, pool, NORM, CGAP |
| | brain, temporal, mw/neuroepithelial tumor, epilepsy, 45M |
| | breast, mw/lobular CA, 58F, m/BRSTTUT03 |
| | colon tumor, adenoCA, pool, NORM, 3′ CGAP |
| | colon, cecum polyp, aw/adenoCA, 67F |
| | colon, mw/adenoCA, aw/node mets, 60M, NORM, m/COLNTUT16 |
| | kidney tumor, renal cell CA, 53F, m/KIDNNOT26 |
| | kidney, 64F |
| | lung tumor, squamous cell CA, 64F |
| | microvascular, dermal, endothelial cells, neonatal, M |
| | mixed, (melanocytes, uterus, fetal heart), SUB, CGAP/WM/WN |
| | nose, nasal polyp, eosinophilia, aw/asthma, 78M |
| | ovary, endometriosis, aw/leiomyomata, 39F, NORM, EF |
| | prostate, AH, mw/adenoCA, 65M |
| | sm intestine, ileum, Crohn's, 18F |
| | thymus, aw/parathyroid adenoma, 21M |
| | Jurkat line, T-cell leukemia, M, untreated |
| | aortic smooth muscle line, M |
| | brain tumor, frontal, astrocytoma, 17F |
| | brain, fetal, 23wM |
| | brain, temporal, mw/mets malignant melanoma, 34M |
| | colon, ascending, mw/CUC, 28M |
| | heart, coronary artery, CAD, 46M |
| | lung tumor, adenoCA, 47M |
| | lung, mw/mets osteoSAR, aw/pleura mets, 58M, NORM |
| | muscle, skeletal, aw/Krabbe, 11mF |
| | neuroganglion tumor, ganglioneuroma, 9M |
| | parathyroid tumor, adenoma, M/F, NORM, WM |
| | prostate stroma, fibroblasts, fetal, 26wM, untreated, NORM |
| | prostate, AH, mw/adenoCA, 65M, m/PROSTUT12 |
| | skin, foreskin, melanocytes, M, NORM, WM/WN |
| | thymus, aw/anencephaly, fetal, 17wF |
| | tibia, periosteum, mw/osteoSAR, osteogenesis imperfecta, 20M |
| | uterus tumor, endometrial adenoCA, F, pooled, 3′ CGAP |
| | adrenal gland, 17M |
| | adrenal glands, aw/anencephaly, fetal, 16wF |
| | aorta, adventitia, 48M |
| | brain stem, aw/DMt1, 72M, NORM |
| | brain, allocortex/neocortex, cingulate, aw/CA, 55F, RP |
| | brain, aw/hypoplastic left heart, fetal, 23wM, NORM |
| | brain, aw/hypoplastic left heart, fetal, 23wM, RP |
| | brain, aw/muscular atrophy, 3mF, NORM, GEXP |
| | brain, globus pallidus/substantia innominata, aw/CA, 55F, RP |
| | brain, hippocampus, 74M, TIGR |
| | brain, hippocampus, aw/CHF, 35M |
| | brain, hippocampus, mw/intracranial hemorrhage, 72F |
| | brain, medulla, aw/CHF, 35M, AMP |
| | brain, mw/oligoastrocytoma, epilepsy, 26M, NORM |
| | brain, pineal gland, TIGR |
| | brain, pineal gland, aw/AD, 68, 79F, pool, NORM |
| | brain, temporal cortex, aw/CHF, 35M |
| | brain, thalamus, aw/CHF, 35M, NORM |
| | breast tumor, adenoCA, 55F, m/BRSTNOT02 |
| | breast tumor, lobular CA, 59F, m/BRSTNOR01, BRSTNOT16 |
| | breast, F, NORM, WM |
| | clavicle, osteoblasts, 40M, untreated, lg cDNA, EF |
| | colon tumor, CA, pool, LICR, EF |
| | colon tumor, adenoCA, 65F, EF |
| | colon tumor, adenoCA, carcinoid, M/F, pool, NORM |
| | colon tumor, rectum, adenoCA, mw/tubular adenoma, 50M, 5RP |
| | colon tumor, sigmoid, adenoCA, 62M, m/COLNNOT16 |
| | colon, aw/anencephaly, fetal, 20wF |
| | colon, normal/pseudopolyp, Crohn, 16, 26M, pool, lg/N |
| | epiglottis, aw/papillary thyroid CA, 71M |
| | esophagus tumor, adenoCA, 61M, NORM |
| | eye tumor line, retinoblastoma, pool, MGC, EF |
| | fat, mesentery, aw/diverticulosis, diverticulitis, 71M |
| | femoral artery, aw/chondroSAR, 68M |
| | gallbladder tumor, squamous cell CA, 78F |
| | gallbladder, cholecystitis, cholelithiasis, 55F, 5RP |
| | ganglion, dorsal root, thoracic, aw/lymphoma, 32M, NORM |
| | ganglion, dorsal root, thoracic/lumbar, aw/lymphoma, 32M |
| | germ cell tumor, pool, SUB, 3′ CGAP |
| | heart, aorta, 39M, lg cDNA, EF |
| | heart, fetal, 18wM |
| | heart, fetal, 8-10w, pool, BI |
| | kidney epithelial transf embryo line, 293-EBNA, t/5AZA |
| | kidney tumor line, CA/adenoCA/hypernephroma, pool, MGC, EF |
| | kidney tumor, clear cell type cancer, pool, NORM, 3′ CGAP |
| | kidney, cortex, mw/renal cell CA, 65M |
| | kidney, interstitial nephritis, aw/bladder TC CA, 63M, 5RP |
| | kidney, mw/renal cell CA, 8, 53F, pool, NORM |
| | kidney, pool, NORM, 3′ CGAP |
| | liver, 49M, WM |
| | liver/spleen, fetal, 20wM, NORM, CGAP/WM/WN |
| | lung tumor line, sm/lg cell CA/mucoepidermoid CA, pool, MGC, EF |
| | lung tumor, neuroendocrine carcinoid, pool, SUB, CGAP |
| | lung tumor, pleura, mets uterine leiomyoSAR, 55F |
| | lung, NORM |
| | lung, asthma, 17M |
| | lung, mw/adenoCA, 63M |
| | lung, mw/adenoCA, aw/node, diaphragm mets, 63F |
| | lung, mw/mets osteoSAR, aw/pleura mets, 58M |
| | lymph node, 16mM |
| | mammary, epithelial cells, 21F, untreated |
| | mixed tissues, fetal lung, testis, B-cell, SUB, 3′ CGAP/WN |
| | mixed tissues, includes tumor, SUB, 3′ CGAP |
| | mixed tissues, includes tumor, pool, SUB, CGAP |
| | nasal/cribriform tumor, olfactory neuroblastoma, 45M, 5RP |
| | ovary tumor, fibrothecoma, F, pool, NORM, 3′, CGAP |
| | pancreas tumor, adenoCA, 3′ CGAP |
| | pancreatic tumor, anaplastic CA, 45F |
| | periph blood, promonocyte line, THP-1, AML, untreated |
| | placenta, aw/hydrocephalus, fetal, 16w, FL-EN, EF |
| | placenta, aw/hydrocephalus, fetal, 16w/18wM, pool, RP |
| | prostate tumor, CA, pool, M, LICR, EF |
| | prostate tumor, adenoCA, 61M |
| | prostate tumor, adenoCA, 66M, m/PROSNOT15, PROSDIN01 |
| | prostate, AH, mw/adenoCA, 57M, 5RP |
| | prostate, AH, mw/adenoCA, 66M, NORM, m/PROSTUT10 |
| | prostate, AH, mw/adenoCA, 69M, m/PROSTUT05 |
| | prostate, AH, mw/adenoCA, M, m/PROSTUT13, PROSTUS08/19/20/25 |
| | seminal vesicle, aw/prostate adenoCA, 63M, 5RP |
| | skin, dermis, breast, fibroblasts, 31F, t/9CIS RA-20 hr |
| | skin, dermis, breast, fibroblasts, 31F, t/9CIS RA-20 hr, SUB |
| | skin, dermis, breast, fibroblasts, 31F, untreated |
| | sm intestine, fetal, 8-16M/F, pool, NORM |
| | spinal cord, aw/renal failure, 71M |
| | spinal cord, cervical, aw/lymphoma, 32M |
| | spleen, 2M |
| | stomach tumor, adenoCA, poorly differentiated, 3′ CGAP |
| | stomach, fetal, 20wF |
| | teratoCA line, NT2, t/5AZA-3d |
| | teratoCA line, hNT2, untreated |
| | testis, 16M |
| | testis, M, NORM, CGAP/WN |
| | thymus, 3M |
| | thymus, aw/Down, 4mM |
| | uterus, F, NORM, CGAP/WM/WN |
| | uterus, aw/ovarian follicular cysts, 34F |
| | uterus, mixed, endometrium/myometrium, 32, 45F, pool, RP |
| | PBMC, 60M, untreated |
| | adrenal gland, 20M, NORM |
| | adrenal tumor, pheochromocytoma, 57F |
| | bone marrow tumor line, SH-SY5Y, pool, Untx/t/6OHDA, NORM, EF |
| | bone marrow, CD34+ hematopoietic stem cells, pool, CHGC, EF |
| | bone tumor, sacrum, giant cell tumor, 18F |
| | brain tumor, frontal, astrocytoma, 40F, m/BRAINOT14 |
| | brain tumor, frontal, meningioma, 61F |
| | brain tumor, frontal, meningioma, 68M |
| | brain tumor, frontal, neuronal neoplasm, 32M |
| | brain tumor, frontal, oligoastrocytoma, 50F |
| | brain tumor, glioblastoma, pool, NORM, CGAP |
| | brain tumor, mixed, pool, MGC, EF |
| | brain, cerebellum, aw/bronchial CA, 64M |
| | brain, frontal cortex, aw/CHF, 35M |
| | brain, frontal cortex, aw/CHF, 35M, NORM |
| | brain, frontal, mw/astrocytoma, 40F, m/BRAITUT12/BRAFTUE03 |
| | brain, hippocampus, aw/atherosclerosis, CHF, 81F |
| | brain, medulla, ALS, 74F |
| | brain, medulla, aw/CHF, 35M |
| | brain, mixed tissues, aw/CHF, 35M, pool, lg/N |
| | brain, pineal gland, aw/AD, COPD, 79F |
| | brain, temporal, polymicrogyria, gliosis, 5M |
| | breast tumor line, adenoCA, MGC, EF |
| | breast tumor, ductal, F, pool, NORM, 3′/5′ CGAP |
| | breast, NF breast disease, 46F |
| | cervical tumor line, HeLa, adenoCA, 31F, t/Na butyrate 24 hr |
| | cervical tumor line, HeLa, adenoCA, 31F, untreated |
| | colon tumor epithelial line, T84, CA, WM |
| | colon tumor, adenoCA, pool, NORM, 3′/5′ CGAP |
| | colon, appendix, aw/leiomyomata, 37F |
| | colon, mixed tissues, mw/Burkitt, 13-37F, pool, lg cDNA |
| | fibroblasts, senescent, NORM, WM/WN |
| | germ cell tumor, seminoma, teratoma adenoCA, pool, 3′ CGAP |
| | heart, 44M, NORM |
| | heart, left ventricle, mw/myocardial infarction, 56M |
| | kidney epithelial transf embryo line, 293-EBNA, serum starv |
| | liver/spleen, fetal, 20wM, NORM, WM |
| | lung, MGC, EF |
| | lung, aw/Patau's, fetal, 20wM |
| | lung, fetal, 19w, NORM, CGAP/WM/WN |
| | lymph node, B-lymphocytes, germinal center, pool, MGC, EF |
| | lymph nodes, 16mM, NORM |
| | microvascular, dermal, endothelial cells, 22F, t/bFGF, EF |
| | mixed tissues, includes tumor, 8-69M/F, pool, NORM, EF |
| | muscle, calf, mw/gangrene, aw/atherosclerosis, 67M |
| | ovary tumor, adenoCA, 58F |
| | pancreatic tumor line, adenoCA, untreated, WM/WN |
| | periph blood, B-lymphocytes, CLL, pool, NORM, 3′ CGAP |
| | placenta, fetal, 21wF |
| | probable astrocytes, M/F, untreated |
| | probable periph blood, eosinophils, asthma, M/F |
| | prostate tumor line, LNCaP, CA, 50M, untreated |
| | prostate tumor, TC CA, 66M, EF |
| | prostate, 28M |
| | prostate, 28M, NORM |
| | prostate, AH, mw/adenoCA, 50M, m/PROSTUT01 |
| | prostate, AH, mw/adenoCA, 55M, m/PROSTUT16 |
| | prostate, AH, mw/adenoCA, 58M |
| | prostate, AH, mw/adenoCA, 67M, m/PROSTUT03 |
| | prostate, epithelium, PIN, mw/cancer, 45M, m/PROETUP02, CGAP |
| | prostate, pool, M, LICR, EF |
| | skin, leg, erythema nodosum |
| | sm intestine, duodenum, aw/pancreatic cystadenoma, 41F |
| | teratoCA line, hNT2, t/RA + MI, WM/WN |
| | tonsil, B-lymphocytes, germinal, aw/tonsillitis, NORM, CGAP |
| | uterus tumor, leiomyoma, m/UTRSNON03, UTRSNOT12, UTRSTMR01 |
| | uterus, aw/liver & breast cancer, 46F |
| | uterus, endometrium, aw/cystocele, 38F |
| | uterus, endometrium, mw/cervical dysplasia, 32F, 5RP |
| | uterus, endometrium, type II defect, endometriosis, F |
| | lymphod |
| | brain |
| #740787: | adipose tumor, lipoSAR, 3′ CGAP |
| | adrenal tumor, pheochromocytoma, 52F, EF |
| | aortic smooth muscle line, M |
| | bladder, mw/TC CA, aw/prostate TC CA, 66M, m/BLADTUT05 |
| | bone marrow stroma, M/F, pool, WG |
| | bone tumor, Ewing's SAR, CGAP |
| | bone tumor, sacrum, giant cell tumor, 18F |
| | brain, 55M, NORM, WM |
| | brain, astrocytes, fetal, 22wF, untreated |
| | brain, globus pallidus/substantia innominata, aw/CA, 55F, RP |
| | breast, mw/lobular CA, 67F |
| | bronchus, smooth muscle cells, 21M, untreated |
| | cartilage, knee, chondrocytes, M/F, t/IL-1 |
| | cervical tumor line, HeLa S3, adenoCA, 31F, untreated, WM/WN |
| | clavicle, osteoblasts, 40M, untreated, lg cDNA, EF |
| | colon tumor, adenoCA, 3′, CGAP |
| | colon tumor, adenoCA, carcinoid, M/F, pool, NORM |
| | colon, TIGR |
| | colon, aw/Patau's, fetal, 20wM, lg cDNA |
| | colon, sigmoid, mw/Crohn's, carcinoid, 40M, m/COLNCRT01 |
| | coronary artery, smooth muscle cells, 3M, NORM |
| | coronary artery, smooth muscle cells, 3M, t/TNF, IL-1 |
| | coronary artery, smooth muscle cells, 3M, t/TNF, IL-1, SUB |
| | coronary artery, smooth muscle cells, CASMC, t/TNFa, 5C-FL, EF |
| | fibroblasts, senescent, NORM, WM/WN |
| | ganglion, dorsal root, cervical, aw/lymphoma, 32M |
| | ganglion, dorsal root, cervical, aw/lymphoma, 32M, NORM, EF |
| | ganglion, dorsal root, thoracic, aw/lymphoma, 32M |
| | ganglion, dorsal root, thoracic/lumbar, aw/lymphoma, 32M |
| | heart tumor, left atrium, myxoma, 43M |
| | heart, aw/Patau's syndrome, fetal, 20wM, 5RP |
| | heart, fetal, 18wM |
| | heart, fetal, 19w, NORM, CGAP/WM/WN |
| | heart, fetal, 8-10w, pool, BI |
| | kidney tumor, Wilms', pool, WM/WN |
| | kidney, cortex, mw/renal cell CA, 65M, 5RP |
| | kidney, pool, NORM, 3′ CGAP |
| | kidney, pool, SUB, 3′ CGAP |
| | liver/spleen, fetal, 20wM, NORM, CGAP/WM/WN |
| | liver/spleen, fetal, 20wM, NORM, WM |
| | lung, aw/Patau's, fetal, 20wM, lg cDNA |
| | lung, mw/mets osteoSAR, aw/pleura mets, 58M |
| | mixed tissues, fetal lung, testis, B-cell, SUB, 3′ CGAP/WN |
| | mixed tissues, includes tumor, SUB, 3′ CGAP |
| | mixed tissues, includes tumor, pool, SUB, CGAP |
| | mixed tissues, myometrium, smooth muscle cells, 57M/41F, B |
| | mixed, (melanocytes, uterus, fetal heart), SUB, CGAP/WM/WN |
| | neural tumor, schwannoma, pool, 3′ CGAP |
| | neuroganglion tumor, ganglioneuroma, 9M |
| | ovary tumor, mucinous cystadenoma, 51F |
| | ovary tumor, serous papillary, F, CGAP |
| | ovary, aw/cardiomyopathy, 59F |
| | pancreas tumor, adenoCA, 3′ CGAP |
| | pancreatic tumor, anaplastic CA, 45F |
| | parathyroid tumor, adenoma, M/F, NORM, WM |
| | placenta, aw/hydrocephalus, fetal demise, 16w, 18wM, pool |
| | placenta, aw/hydrocephalus, fetal, 16w, 5RP |
| | placenta, aw/hydrocephalus, fetal, 16w, RP |
| | placenta, aw/hydrocephalus, fetal, 16w/18wM, pool, RP |
| | placenta, fetal, 18wM |
| | placenta, fetal, 21wF |
| | placenta, fetal, 3′ TIGR |
| | placenta, fetal, 8-9w, pool, NORM, CGAP/WM |
| | placenta, fetal, M, WM |
| | placenta, fetal, TIGR |
| | placenta, neonatal, F |
| | placenta, neonatal, F, NORM, WM |
| | prostate stroma, fibroblasts, fetal, 26wM, untreated |
| | pulmonary artery, endothelial cells, 10M, t/TNF, IL-1 |
| | renal vein, smooth muscle cells, 57M t/TNF, IL1 |
| | skin, dermis, breast, fibroblasts, 31F, t/9CIS RA-20 hr |
| | sm intestine, ileum, aw/adenoCA cecum, node mets, 70F |
| | soft tissue tumor, spinal schwannoma, 35M |
| | spleen, Gaucher's, 22M |
| | stomach, 16M |
| | stomach, gastritis, mw/adenoCA, node mets, 76M, RP |
| | teratoCA line, hNT2, t/RA + MI, WM |
| | testis, 16M, NORM |
| | testis, M, NORM, CGAP/WN |
| | thymus, hyperplasia, aw/myasthenia gravis, 16F |
| | thyroid, mw/follicular adenoma, 28F |
| | tongue, squamous epithelium, mw/CA, m/TONGTUP01, CGAP |
| | uterus tumor, leiomyoma, m/UTRSNON03, UTRSNOT12, UTRSTMR01 |
| | uterus, F, NORM, CGAP/WM/WN |
| | uterus, endometrium, mw/leiomyoma aw/endometriosis, 48F |
| | uterus, endometrium, type I defect, 30, 32, 36F, pool |
| | uterus, mw/leiomyoma, aw/colon adenoCA, 45F |
| | uterus, myometrium, mw/leiomyoma, 41F, RP, m/UTRSTUT05 |
| | placenta |
| #42024: | brain, aw/muscular atrophy, 3mF, NORM, GEXP |
| | brain, hypothalamus, Huntington's, mw/CVA, 57M, NORM |
| | brain, striatum/globus pallidus/putamen, aw/CHF, 81F, RP |
| | breast tumor, CA, pool, LICR, EF |
| | liver/spleen, fetal, 20wM, NORM, WM |
| | lung tumor line, NCI-H69, sm cell CA, 55M, untreated, WM/WN |
| | lung tumor line, sm/lg cell CA/mucoepidermoid CA, pool, MGC, EF |
| | skin, foreskin, melanocytes, M, NORM, WM/WN |
| | ureter tumor, TC CA, 64M, 5RP |
| | aorta, aw/cerebral agenesis, 10M/27F, pool, RP |
| | brain, aw/hypoplastic left heart, fetal, 23wM, FL-EN, EF |
| | brain, aw/hypoplastic left heart, fetal, 23wM, lg cDNA |
| | brain, corpus callosum, AD, 74M |
| | brain, frontal, gliosis, mw/epilepsy, cerebral palsy, 27M |
| | brain, infant, 10wF, NORM, WM |
| | brain, pineal gland, aw/AD, 68, 79F, pool, NORM |
| | brain, temporal cortex, schizophrenia, aw/COPD, 55M |
| | breast, F, NORM, WM |
| | breast, mw/lobular CA, 58F, m/BRSTTUT03 |
| | cartilage, M/F |
| | colon tumor epithelial line, T84, CA, WM |
| | colon, cecum, Crohn's, 31M |
| | heart, fetal, 19w, NORM, CGAP/WM/WN |
| | heart, fetal, 8-10w, pool, BI |
| | kidney tumor, clear cell type cancer, pool, NORM, 3′ CGAP |
| | kidney tumor, renal cell CA, pool, 3′/5′ CGAP |
| | kidney, MGC, EF |
| | kidney, aw/anencephaly, fetal, 17wF |
| | kidney, fetal, 19-23w, M/F, lg cDNA |
| | kidney, right/left, aw/Patau's, fetal, 20wM, pool, lg cDNA |
| | liver tumor line, C3A, Hepatob, 15M, t/MCA-48 hr, SUB |
| | lung tumor, squamous cell CA, 56M, 5RP |
| | lung tumor, squamous cell CA/mets liposarcoma, pool, SUB, EF |
| | lung, aw/anencephaly, fetal, 20wF |
| | mixed, (melanocytes, uterus, fetal heart), SUB, CGAP/WM/WN |
| | nervous tissue, pool, LICR, EF |
| | ovary, endometriosis, aw/leiomyomata, 39F |
| | pituitary, 16-70M/F, pool |
| | placenta, aw/hydrocephalus, fetal demise, 16w, 8wM, pool |
| | placenta, pool, LICR, EF |
| | skull tumor, chondroid chordoma, 30F |
| | spinal cord, base medulla, Huntington's, 57M, lg cDNA, EF |
| | urogenital tumor, TC CA, pool, 3′ CGAP |
| | uterus, aw/ovarian follicular cysts, 34F |
| | uterus, endometrium, mw/cervicitis, leiomyoma, pool, lg cDNA |
| | colon |
| | hepatoma (HepG2) |
| | Lung, small cell carcinoma |
| #43542: | Jurkat line, T-cell leukemia, M, t/PMA, Iono-1 hr, 5C-FL, EF |
| | T-lymphocyte tumor, lymphoma, TIGR |
| | brain, striatum/globus pallidus/putamen, aw/CHF, 81F, RP |
| | brain, substantia nigra, aw/atherosclerosis, CHF, 81F, NORM |
| | breast tumor line, T-47D, ductal CA, 54F, Untx, NORM, EF |
| | colon tumor, adenoCA, 3′ CGAP |
| | colon, aw/Krabbe, 11mF, FL-EN, EF |
| | fallopian tube tumor, endometrioid/serous adenoCA, 85F |
| | fallopian tube tumor, endometrioid/serous adenoCA, 85F, RP |
| | fetus, 8-9w, pool, NORM, CGAP/WM/WN |
| | kidney tumor line, CA/adenoCA/hypernephroma, pool, MGC, EF |
| | liver/spleen, fetal, 20wM, NORM, CGAP/WM/WN |
| | lung tumor line, sm/lg cell CA/mucoepidermoid CA, pool, MGC, EF |
| | lung tumor, squamous cell CA, pooled, NORM, CGAP |
| | lymph node tumor line, lymphoma/Burkitt, pool, MGC, EF |
| | lymph node, 11F |
| | lymph node, B-lymphocytes, germinal center, pool, MGC, EF |
| | mixed tissues, fetal lung, testis, B-cell, SUB, 3′ CGAP/WN |
| | nose, nasal polyps, aw/asthma, 78M, pool, NORM |
| | ovary tumor line, adenoCA, pool, MGC, EF |
| | pancreas tumor line, adenoCA/epitheloid CA, pool, MGC, EF |
| | penis, corpus cavernosum, M |
| | periph blood, B-lymphocytes, CLL, pool, NORM, 3′ CGAP |
| | placenta, aw/hydrocephalus, fetal demise, 16w, 18wM, pool |
| | placenta, aw/hydrocephalus, fetal, 16w |
| | placenta, aw/hydrocephalus, fetal, 16w, RP |
| | placenta, neonatal, F, NORM, WM |
| | skin tumor line, melanoma/squamous cell CA, pool, MGC, EF |
| | teratoCA line, hNT2, t/RA |
| | teratoCA line, hNT2, untreated, WM/WN |
| | testis tumor, embryonal CA, 31M, 5RP |
| | tonsil, B-lymphocytes, germinal, aw/tonsillitis, NORM, CGAP |
| | ureter tumor, TC CA, 64M, 5RP |
| | uterus, cervix, cervicitis, mw/leiomyoma, 29F, 5RP |
| | uterus, mixed tissues, mw/tumor, F, pool, lg cDNA, EF |
| | uterus, mixed, endometrium/myometrium, 32, 45F, pool, RP |
| | uterus, myometrium, mw/leiomyoma, 41F, NORM, m/UTRSTUT05 |
| | uterus, myometrium, mw/multiple leiomyomata, 45F |
| | uterus, tumor line, adenoCA/leiomyoSAR, pool, MGC, EF |
| | germ cell tumor, pool, NORM, 3′ CGAP |
| | germ cell tumor, pool, SUB, 3′ CGAP |
| | testis, M, NORM, CGAP/WN |
| | Skin, melanotic melanoma. |
| | hepatoma (HepG2) |
| #37589: | sm intestine, mw/carcinoid, aw/node mets, 59M, RP |
| | uterus tumor, serous papillary CA, F, pooled, 3′ CGAP |
| | uterus, endometrium, type I defect, 30, 32, 36F, pool |
| | fat, breast, aw/fibrosis, 38F |
| | germ cell tumor, pool, SUB, 3′ CGAP |
| | periph blood, macrophages, adher PBMC, M/F |
| | Jurkat line, Frac Nuc, T-cell leukemia, M, t/antiCD3, 5C-FL, EF |
| | brain, cerebellum, Huntington's, mw/CVA, 57M, RP |
| | brain, corpus callosum, Huntington's, mw/CVA, 57M, RP |
| | brain, pons, aw/CHF, 35M |
| | breast, PF changes, mw/ductal adenoCA, 54F, m/BRSTTUT02 |
| | colon tumor, adenoCA, 75M, m/COLNNOT01 |
| | fetus, 8-9w, pool, NORM, CGAP/WM/WN |
| | lung tumor, neuroendocrine carcinoid, pool, SUB, CGAP |
| | penis, corpus cavernosum, M |
| | placenta, aw/hydrocephalus, fetal, 16w, 5RP |
| | placenta, aw/hydrocephalus, fetal, 16w/18wM, pool, RP |
| | sm intestine, ileum, aw/adenoCA cecum, node mets, 70F |
| | spinal cord, base medulla, Huntington's, aw/CVA, 57M |
| | uterus, endometrium, mw/cervical dysplasia, 32F, 5RP |
| | Jurkat line, T-cell leukemia, M, Untx, 5C-FL, EF |
| | colon, epithelium, 13F, RP |
| | nasal/cribriform tumor, olfactory neuroblastoma, 45M, 5RP |
| | CML precursor line, K-562, 53F, t/5AZA-72 hr |
| | Jurkat line, T-cell leukemia, M, t/PMA-30 min, 5C-FL, EF |
| | T-lymphocytes, allogenic anergic, 40-50M, t/OKT3 3 day |
| | adipose, pericecal, aw/cecal tubular adenoma, 55F |
| | adrenal gland, 20M |
| | aorta, adventitia, 48M |
| | aorta, adventitia, 65F |
| | bladder tumor, TC CA, 72M |
| | bone tumor, sacrum, giant cell tumor, 18F |
| | brain tumor, frontal, meningioma, 50M |
| | brain, caudate/putamen/nucleus accumbens, aw/CHF, 35M |
| | brain, choroid plexus, Huntington's, mw/CVA, 57M, RP |
| | brain, frontal cortex, aw/CHF, 35M, NORM |
| | brain, globus pallidus/substantia innominata, aw/CHF, 35M |
| | brain, hippocampus, aw/aortic aneurysm, 45F, 5RP |
| | brain, hippocampus, mw/intracranial hemorrhage, 72F, NORM |
| | brain, mixed tissues, aw/CHF, 35M, pool, lg cDNA |
| | brain, mw/oligoastrocytoma, epilepsy, 26M, NORM |
| | brain, neurogenic tumor line, SK-N-MC, neuroepithelioma, 14F |
| | brain, parietal cortex, aw/CHF, 35M |
| | brain, sensory-motor cortex, aw/CHF, 35M |
| | brain, striatum, caudate nucleus, schizophrenia, 49M |
| | brain, temporal cortex, aw/CHF, 35M |
| | breast tumor, ductal CA, 43F, m/BRSTTMT01 |
| | breast tumor, lobular CA in situ, F, CGAP |
| | breast tumor, lobular CA, 58F, m/BRSTNOT05 |
| | breast, 46F |
| | breast, NF breast disease, 46F |
| | breast, PF changes, mw/adenoCA, intraductal CA, 43F |
| | cartilage, OA, M/F |
| | colon tumor, adenoCA, 60M, m/COLNNOT07/08/09/11 |
| | colon tumor, adenoCA, 64F |
| | colon tumor, adenoCA, pool, NORM, 3′/5′ CGAP |
| | colon tumor, cecum, carcinoid, 30F |
| | colon tumor, sigmoid, adenoCA, 62M, m/COLNNOT16 |
| | colon, aw/Patau's, fetal, 20wM |
| | colon, aw/anencephaly, fetal, 20wF |
| | colon, mw/adenoCA, 55M, m/COLHTUT01, COLHTUS02 |
| | colon, normal/pseudopolyp, Crohn, 16, 26M, pool, lg/N |
| | colon, sigmoid, mw/adenoCA, aw/node mets, 62M, m/COLNTUT03 |
| | fallopian tube tumor, endometrioid/serous adenoCA, 85F, RP |
| | gallbladder, cholecystitis, cholelithiasis, 18F |
| | ganglion, dorsal root, thoracic/lumbar, aw/lymphoma, 32M |
| | heart, aorta, aw/cerebral agenesis, 27F |
| | heart, coronary artery, CAD, 46M |
| | heart, fetal, 18wM |
| | heart, hypoplastic left, fetal, 23wM |
| | iliac artery, endothelial cells, F, t/TNF, IL-1 20 hr |
| | kidney epithelial transf embryo line, 293-EBNA, Untx, 5C-FL, EF |
| | kidney epithelial transf embryo line, 293-EBNA, lg cDNA |
| | kidney, fetal, 19-23w, M/F, lg cDNA |
| | kidney, interstitial nephritis, aw/bladder TC CA, 63M, 5RP |
| | kidney, mw/renal cell CA, 65M, m/KIDNTUT15 |
| | kidney, pool, NORM, 3′ CGAP |
| | kidney, pool, SUB, 3′ CGAP |
| | liver, 49M |
| | lung tumor line, NCI-H69, sm cell CA, 55M, untreated, WM/WN |
| | lung tumor, adenoCA, 53M, m/LUNGNOT28 |
| | lung tumor, mets thyroid CA, 79M, m/LUNGNOT03 |
| | lung tumor, squamous cell CA, 50M |
| | lung tumor, squamous cell CA, 68M |
| | lung, aw/Patau's, fetal, 20wM |
| | lung, aw/anencephaly, fetal, 20wF |
| | lung, mw/caseating granuloma, 58F |
| | lung, mw/mets osteoSAR, aw/pleura mets, 58M, NORM |
| | lymphocytes, activated Th1 cells, 6-hr AB |
| | megakaryoblast line, MEG-01, CML, 55M, untreated |
| | mixed tissues, includes tumor, SUB, 3′ CGAP |
| | mixed tissues, includes tumor, pool, SUB, 3′ CGAP |
| | mixed tissues, includes tumor, pool, SUB, CGAP |
| | mixed tissues, includes tumor, treated cells, pool, NORM, EF |
| | mixed tissues, myometrium, smooth muscle cells, 57M/41F, B |
| | multiple sclerosis, 46M, NORM, WM/WN |
| | muscle, thigh, ALS, 74F, NORM |
| | neuroganglion tumor, ganglioneuroma, 9M |
| | nose, nasal polyp, eosinophilia, aw/asthma, 78M |
| | ovary tumor, TC CA, 53F |
| | ovary tumor, mucinous cystadenoma, 51F |
| | ovary tumor, seroanaplastic CA, 52F |
| | ovary, aw/menorrhagia, 47F, 5RP |
| | ovary, endometriosis, 24F |
| | penis, corpora cavernosa, M |
| | periph blood, eosinophils, nonallergic, M/F |
| | periph blood, promonocyte line, THP-1, AML, 1M, untreated |
| | peritoneum tumor, neuroendocrine CA, 66F |
| | placenta, fetal, 18wM |
| | placenta, fetal, 21wF |
| | placenta, neonatal, F |
| | prostate tumor, adenoCA, 58M |
| | prostate, AH, mw/adenoCA, 55M, m/PROSTUT16 |
| | prostate, AH, mw/adenoCA, 65M |
| | renal vein, smooth muscle cells, 57M, Untx |
| | skin, aw/Patau's, fetal, 20wM |
| | sm intestine tumor, ileum, mets endometrial adenoCA, 64F |
| | sm intestine, aw/Patau's syndrome, fetal, 20wM, 5RP |
| | sm intestine, duodenum, 16M |
| | sm intestine, fetal, M, RP, EF |
| | sm intestine, ileum, mw/CUC, 42M |
| | spinal cord, aw/renal failure, 71M, NORM |
| | spleen, ITP, 14M |
| | testis, 16M |
| | thymus, 3M |
| | thymus, aw/congenital heart abnormalities, 2F |
| | thymus, aw/parathyroid adenoma, 21M |
| | thymus, aw/patent ductus arteriosus, 3M, 5RP |
| | tonsil, B-lymphocytes, germinal, aw/tonsillitis, NORM, CGAP |
| | tonsil, lymphoid hyperplasia, 6M |
| | umb cord vein endothelial line, HUV-EC-C, shear stress |
| | uterus tumor, endometrial adenoCA, F, pooled, 3′ CGAP |
| | uterus tumor, leiomyoma, m/UTRSNON03, UTRSNOT12, UTRSTMR01 |
| | uterus, F, NORM, CGAP/WM/WN |
| | uterus, endometrium, type II defect, endometriosis, F |
| | uterus, myometrium, mw/adenoCA, 59F, RP |
| | uterus, myometrium, mw/leiomyoma, 41F, RP, m/UTRSTUT05 |
| | brain |
| | Brain |
| #42804: | Jurkat line, Frac Nuc, T-cell leukemia, M, t/antiCD3, 5C-FL, EF |
| | fallopian tube tumor, endometrioid/serous adenoCA, 85F |
| | lung, aw/Patau's, fetal, 20wM, lg cDNA |
| | Placenta, choriocarcinoma |
| | hepatoma (HepG2) |
| | CML precursor line, K-562, 53F, t/PMA-96 hr |
| | Jurkat line, T-cell leukemia, untreated, TIGR |
| | T-lymphocyte tumor, lymphoma, TIGR |
| | T-lymphocytes, CD4+, pool, t/anti-CD28 antibodies |
| | UCB, derived dendritic cells, pool, untreated/treated, NORM |
| | aorta, endothelial cells, M |
| | bladder tumor, TC CA, 60M, m/BLADNOT05 |
| | bladder, mw/TC CA, aw/node mets, 58M, m/BLADTUT03 |
| | bone tumor, Ewing's SAR, CGAP |
| | brain tumor, benign meningioma, 35F |
| | brain tumor, benign meningioma, 35F, NORM |
| | brain tumor, frontal, astrocytoma, 17F |
| | brain, acute/chronic multiple sclerosis, pool |
| | brain, astrocytes, fetal, 22wF, t/TNF, IL-1 24 hr |
| | brain, astrocytes, fetal, 22wF, t/carbachol 16 hr |
| | brain, aw/hypoplastic left heart, fetal, 23wM |
| | brain, aw/hypoplastic left heart, fetal, 23wM, NORM |
| | brain, aw/hypoplastic left heart, fetal, 23wM, RP |
| | brain, aw/hypoplastic left heart, fetal, 23wM, lg cDNA |
| | brain, aw/muscular atrophy, 3mF, NORM, GEXP |
| | brain, caudate nucleus, schizophrenia, 66F |
| | brain, caudate/putamen/nucleus accumbens, aw/CHF, 35M |
| | brain, cerebellum, Huntington's, mw/CVA, 57M, RP |
| | brain, cerebellum, aw/bronchial CA, 64M |
| | brain, corpus callosum, AD, 74M |
| | brain, fetal, 23wM |
| | brain, fetal, 23wM, 5C-RP |
| | brain, hippocampus, aw/CHF, 35M |
| | brain, hippocampus, mw/intracranial hemorrhage, 72F, NORM |
| | brain, infant, 10wF, NORM, WM |
| | brain, mixed tissues, aw/cholangioCA, 55F, RP |
| | brain, neurogenic tumor line, SK-N-MC, neuroepithelioma, 14F |
| | brain, occipital, Huntington's, mw/CVA, 57M |
| | brain, pineal gland, aw/AD, 68, 79F, pool, NORM |
| | brain, pineal gland, aw/AD, COPD, 79F |
| | brain, temporal cortex, aw/CHF, 35M |
| | brain, thalamus, aw/CHF, 35M |
| | breast tumor line, T-47D, ductal CA, 54F, Untx, NORM, EF |
| | breast tumor line, adenoCA, MGC, EF |
| | breast tumor, CA, pool, LICR, EF |
| | breast tumor, adenoCA, 46F, m/BRSTNOT33, EF |
| | breast tumor, high vascular density, CA, F |
| | breast, 46F |
| | breast, 56F |
| | breast, F, NORM, WM |
| | breast, NF breast disease, 32F |
| | breast, PF breast disease, 57F |
| | breast, PF changes, mw/adenoCA, 55F, m/BRSTTUT01 |
| | breast, PF changes, mw/intraductal cancer, 48F |
| | breast, mw/ductal adenoCA, intraductal CA, aw/node mets, 57F |
| | breast, mw/lobular CA, 58F, m/BRSTTUT03 |
| | breast, papillomatosis, mw/lobular CA, 59F, m/BRSTTUT22 |
| | breast, pool, LICR, EF |
| | bronchial epithelium line, NHBE, 54M, Untx |
| | bronchial, epithelial cells, 23M, t/20% smoke 20 hr |
| | colon tumor, CA, pool, LICR, EF |
| | colon tumor, adenoCA, 75M, m/COLNNOT01 |
| | colon tumor, adenoCA, NORM, SUB, CGAP |
| | colon tumor, adenoCA, pool, NORM, 3′ CGAP |
| | colon tumor, adenoCA, pool, NORM, 3′/5′ CGAP |
| | colon, 16M |
| | colon, 25M, WN |
| | colon, ascending, CUC, 25F |
| | colon, aw/Patau's, fetal, 20wM, lg cDNA |
| | colon, cecum, Crohn's, 31M |
| | colon, cecum/descending, polyposis, polyp, M/F, pool, SUB |
| | colon, sigmoid, mw/adenoCA, aw/node mets, 62M, m/COLNTUT03 |
| | colon, ulcerative colitis, 16M |
| | eye, retina, 55M, NORM, WM |
| | fallopian tube tumor, endometrioid/serous adenoCA, 85F, 5RP, EF |
| | fetus, 12w, TIGR |
| | fetus, 8-9w, pool, NORM, CGAP/WM/WN |
| | ganglion, dorsal root, cervical, aw/lymphoma, 32M |
| | ganglion, dorsal root, cervical, aw/lymphoma, 32M, NORM, EF |
| | ganglion, dorsal root, thoracic, aw/lymphoma, 32M, NORM |
| | germ cell tumor, pool, SUB, 3′ CGAP |
| | heart tumor, left atrium, myxoma, 43M |
| | heart, 44M, NORM |
| | heart, aorta, aw/cerebral agenesis, 27F |
| | heart, coronary artery, endothelial cells, 3M, untreated |
| | heart, fetal, 19w, NORM, CGAP/WM/WN |
| | heart, fetal, M |
| | hypothalamus, 16-75M/F |
| | kidney tumor, clear cell type cancer, pool, NORM, 3′ CGAP |
| | kidney tumor, clear cell type cancer, pool, SUB, CGAP |
| | kidney, 2dF |
| | kidney, 8M |
| | kidney, medulla/cortex, mw/renal cell CA, 53F, m/KIDNTUT16 |
| | kidney, mw/renal cell CA, 65M, m/KIDNTUT15 |
| | kidney, mw/renal cell CA, 8, 53F, pool, NORM |
| | kidney, pool, SUB, 3′ CGAP |
| | liver tumor line, C3A, Hepatob, 15M, Untx |
| | liver tumor line, C3A, Hepatob, 15M, Untx, NORM |
| | liver, hepatitis C, 35F |
| | liver/spleen, fetal, 20wM, NORM, CGAP/WM/WN |
| | liver/spleen, fetal, 20wM, NORM, WM |
| | lung tumor, adenoCA, 70F |
| | lung tumor, neuroendocrine carcinoid, pool, NORM, 3′ CGAP |
| | lung tumor, neuroendocrine carcinoid, pool, SUB, CGAP |
| | lung tumor, squamous cell CA, 57M |
| | lung tumor, squamous cell CA, pooled, NORM, CGAP |
| | lung, 2M |
| | lung, asthma, 17M |
| | lung, aw/anencephaly, fetal, 17wF |
| | lung, fetal, 19w, NORM, CGAP/WM/WN |
| | lung, mw/mets osteoSAR, aw/pleura mets, 58M, NORM |
| | lung, mw/spindle cell carcinoid, 62F |
| | lung, right bronchus, nonasthmatic, 18-55M/F, pool |
| | lymph nodes, 16mM, NORM |
| | lymphocytes, PBMC, M, 96-hr MLR |
| | lymphocytes, activated Th1 cells, 6-hr AB |
| | lymphocytes, activated Th2 cells, 6-hr AB |
| | lymphocytes, nonactivated Th1 cells |
| | microvascular, dermal, endothelial cells, 18F, untreated |
| | mixed tissues, fetal lung, testis, B-cell, SUB, 3′ CGAP/WN |
| | mixed tissues, includes tumor, SUB, 3′ CGAP |
| | mixed tissues, includes tumor, pool, SUB, 3′ CGAP |
| | mixed tumor, Wilm's/brain mets, pool, 3′ CGAP |
| | mixed, (melanocytes, uterus, fetal heart), SUB, CGAP/WM/WN |
| | mixed, brain/pituitary, Nrml/Huntington's/AD, M/F, pool, 5RP, EF |
| | multiple sclerosis, 46M, NORM, WM/WN |
| | nervous tissue, pool, LICR, EF |
| | ovary tumor, mets colon adenoCA, 58F, NORM |
| | ovary tumor, mixed types, pooled, F, 3′ CGAP |
| | ovary tumor, seroanaplastic CA, 52F |
| | ovary, aw/leiomyomata, 47F |
| | ovary, mw/mucinous cystadenoCA, 43F, m/OVARTUT01 |
| | pancreas tumor, adenoCA, 3′ CGAP |
| | pancreas, fetal, 23wM |
| | pancreas, islet cells, pool |
| | penis, glans, aw/scrotal urothelial CA, M |
| | periph blood, B-lymphocytes, CLL, pool, NORM, 3′ CGAP |
| | periph blood, eosinophils, asthma, M/F |
| | periph blood, eosinophils, t/IL-5 |
| | periph blood, lymphocytes, non-adher PBMC, M/F, 24-hr MLR |
| | periph blood, monocytes, 42F, t/IL-10, LPS |
| | pituitary gland, aw/AD, mets adenoCA, 70F, RP |
| | pituitary tumor, adenoma, pool, 3′, CGAP |
| | placenta, neonatal, F, NORM, WM |
| | placenta, pool, LICR, EF |
| | probable astrocytes, M/F, untreated |
| | probable periph blood, eosinophils, asthma, M/F |
| | prostate epithelium line, M, untreated, 3′ CGAP |
| | prostate tumor, adenoCA, 58, 61, 66, 68M, pool, SUB |
| | prostate tumor, adenoCA, 59M, SUB, m/PROSNOT19 |
| | prostate tumor, adenoCA, 69M, m/PROSNOT07 |
| | prostate, 28M, NORM |
| | prostate, 32M, SUB, 3′ CGAP |
| | prostate, AH, mw/adenoCA, 66M, m/PROSTUT10 |
| | prostate, AH, mw/adenoCA, 66M, m/PROSTUT17 |
| | seminal vesicle, aw/adenoCA, 58M |
| | seminal vesicle, aw/prostate adenoCA, 63M, 5RP |
| | skin, epidermis, keratinocytes, neonatal, M, t/TNF, IL-1 |
| | skin, foreskin, melanocytes, M, NORM, WM/WN |
| | sm intestine tumor, ileum, mets endometrial adenoCA, 64F |
| | sm intestine, 8M |
| | sm intestine, ileum, mw/CUC, 42M |
| | soft tissue, retroperitoneal/supraglottic, pool, AMP |
| | spinal cord, aw/renal failure, 71M |
| | stomach tumor, CA, pool, LICR, EF |
| | stomach tumor, adenoCA, poorly differentiated, 3′ CGAP |
| | stomach, pool, LICR, EF |
| | teratoCA line, hNT2, t/RA + MI |
| | testis, M, NORM, CGAP/WN |
| | thymus, 3M |
| | thymus, aw/parathyroid adenoma, 21M |
| | thymus, aw/patent ductus arteriosus, 3M |
| | thymus, aw/patent ductus arteriosus, 3M, 5RP |
| | thymus, hyperplasia, aw/myasthenia gravis, 16F |
| | thyroid, pool, LICR, EF |
| | tonsil, B-lymphocytes, germinal, aw/tonsillitis, NORM, CGAP |
| | umb cord blood, T-lymphocytes, Th2 cells, untreated |
| | uterus tumor, CA, pool, LICR, EF |
| | uterus tumor, endometrial adenoCA, F, pooled, 3′ CGAP |
| | uterus tumor, leiomyoma, m/UTRSNON03, UTRSNOT12, UTRSTMR01 |
| | uterus, F, NORM, CGAP/WM/WN |
| | uterus, aw/ovarian follicular cysts, 34F |
| | uterus, cervix tumor line, CA, pool, MGC, EF |
| | uterus, endometrium, F, pool |
| | uterus, endometrium, aw/adenoCA, 50F |
| | uterus, endometrium, mw/cervical dysplasia, 32F |
| | uterus, endometrium, mw/cervical dysplasia, 32F, FL-EN, EF |
| | uterus, endometrium, mw/leiomyoma, 29F, NORM |
| | uterus, endometrium, mw/leiomyoma, 29F, RP |
| | uterus, myometrium, mw/leiomyoma, 43F |
| | liver, MGC, EF |
| | prostate, AH, mw/adenoCA, 48-73M, pool, lg cDNA |
| | Raji line, Burkitt, 11M, t/PMA, Iono-30 min, 5C-FL, EF |
| | bladder tumor, TC CA, 80F, m/BLADNOT03 |
| | brain tumor line, DU 145, mets prostate CA, 69M, Untx, 5C-FL, EF |
| | brain tumor, frontal, astrocytoma, 40F, m/BRAINOT14 |
| | brain, archaeocortex, hippocampus, aw/cholangioCA, 55F, RP |
| | breast tumor line, T-47D, ductal CA, 54F, 5C-FL, EF |
| | breast, NF breast disease, 35F |
| | colon tumor epithelial line, T84, CA, WM |
| | embryo, 8w, TIGR |
| | kidney, fetal, 19-23w, M/F, lg cDNA |
| | mixed tissues, includes tumor, pool, SUB, CGAP |
| | periph blood, lymphocytes, non-adher PBMC, 24M |
| | prostate stroma, fibroblasts, fetal, 26wM, untreated, NORM |
| | testis tumor, embryonal CA, 31M, EF |
| | uterus, tumor line, adenoCA/leiomyoSAR, pool, MGC, EF |
| | Breast, mammary adenocarcinoma. |
| | ileal mucosa |
| | colon |
| #41590: | adrenal gland, epithelium, 16wM, TIGR |
| | brain tumor, benign meningioma, 35F |
| | brain tumor, frontal, astrocytoma, 40F, 5RP, m/BRAINOT14 |
| | brain, allocortex/neocortex, cingulate, aw/CA, 55F, RP |
| | brain, aw/muscular atrophy, 3mF, NORM, GEXP |
| | brain, choroid plexus, Huntington's, mw/CVA, 57M, RP |
| | brain, globus pallidus/substantia innominata, aw/CHF, 35M |
| | brain, infant, 10wF, NORM, WM |
| | brain, mixed tissues, aw/cholangioCA, 55F, RP |
| | brain, neocortex, parietal, aw/cholangioCA, 55F, RP |
| | breast tumor, lobular CA, 58F, m/BRSTNOT05 |
| | colon line, pool, LICR, EF |
| | colon tumor line, KM12C, CA, Untx, TIGR |
| | colon, aw/Patau's, fetal, 20wM, lg cDNA |
| | colon, cecum, benign familial polyposis, 16M |
| | colon, cecum/descending, polyposis, polyp, M/F, pool, SUB |
| | colon, descending, benign familial polyposis, 16M |
| | colon, normal/pseudopolyp, Crohn, 16, 26M, pool, lg/N |
| | ganglion, dorsal root, thoracic, aw/lymphoma, 32M, NORM |
| | ganglion, dorsal root, thoracic/lumbar, aw/lymphoma, 32M |
| | heart, 44M, NORM |
| | heart, aw/Patau's syndrome, fetal, 20wM, 5RP |
| | heart, fetal, 8-10w, pool, BI |
| | kidney, 2dF |
| | kidney, aw/anencephaly, fetal, 17wF |
| | kidney, fetal, 19-23w, M/F, lg cDNA |
| | kidney, right/left, aw/Patau's, fetal, 20wM, pool, lg cDNA |
| | liver tumor line, C3A, Hepatob, 15M, t/insulin-24 hr, 5C-FL, EF |
| | liver/spleen, fetal, 20wM, NORM, WM |
| | lung tumor, neuroendocrine carcinoid, pool, SUB, CGAP |
| | lung, 17F, FL-EN, EF |
| | lung, 72M, WM/WN |
| | lung, aw/Patau's, fetal, 20wM, lg cDNA |
| | lung, fetal, 19w, NORM, CGAP/WM/WN |
| | lung, fetal, 19wM, TIGR |
| | lung, mw/caseating granuloma, 58F |
| | lung, pool, LICR, EF |
| | mixed tissue, head/neck, pool, LICR, EF |
| | mixed tissues, includes tumor, 23-66M, pool, NORM, EF |
| | mixed tissues, includes tumor, treated cells, pool, NORM, EF |
| | nervous tissue, pool, LICR, EF |
| | olfactory bulb, aw/CA, 39-85M/F, pool, NORM, EF |
| | ovary, aw/cardiomyopathy, 59F, NORM |
| | pancreas, type I diabetes, 15M |
| | pituitary gland, pool, CHGC, EF |
| | placenta, aw/hydrocephalus, fetal demise, 16w, 18wM, pool |
| | placenta, aw/hydrocephalus, fetal, 16w, 5RP |
| | placenta, aw/hydrocephalus, fetal, 16w, RP |
| | placenta, fetal, 3′ TIGR |
| | placenta, fetal, 8-9w, pool, NORM, CGAP/WM |
| | prostate, 28M |
| | skin, epidermal breast keratinocyte line, NHEK, 30F, Untx |
| | skin, leg, keratinocytes, neonatal, M |
| | sm intestine, ileum, mw/carcinoid, 30F |
| | teratoCA line, hNT2, t/RA |
| | testis, M, NORM, CGAP/WN |
| | uterus tumor, endometrial adenoCA, F, pooled, 3′ CGAP |
| | uterus tumor, endometrial, F, TIGR |
| | uterus, myometrium, mw/multiple leiomyomata, 45F |
| | uterus, tumor line, adenoCA/leiomyoSAR, pool, MGC, EF |
| | brain, aw/hypoplastic left heart, fetal, 23wM, 5RP |
| | ovary, aw/menorrhagia, 47F, 5RP |
| | sm intestine, aw/stomach ulcer, 49F, 5RP |
| #45900: | breast tumor, adenoCA, 55F, m/BRSTNOT02 |
| | kidney, 8M |
| | placenta tumor line, chorioCA, fetal, pool, MGC, EF |
| | uterus tumor, endometrium, adenosquamous CA, 49F, 5RP |
| | aorta, adventitia, 48M |
| | bladder tumor, TC CA, 80F, m/BLADNOT03 |
| | colon, mw/adenoCA, aw/node mets, 60M, m/COLNTUT16 |
| | fallopian tube tumor, endometrioid/serous adenoCA, 85F |
| | fat, mixed tissues, aw/breast adenoCA, 38-73M/F, pool, NORM |
| | gallbladder, cholecystitis, cholelithiasis, 53F |
| | heart, left ventricle, 31M |
| | kidney, pool, SUB, 3′ CGAP |
| | liver, primary biliary cirrhosis, 63F |
| | lung tumor line, sm/lg cell CA/mucoepidermoid CA, pool, MGC, EF |
| | lung tumor, squamous cell CA, pooled, NORM, CGAP |
| | mixed tissues, includes tumor, SUB, 3′ CGAP |
| | pancreatic tumor, anaplastic CA, 45F |
| | prostate tumor, adenoCA, 58, 61, 66, 68M, pool, SUB |
| | prostate tumor, adenoCA, 59M, m/PROSNOT19 |
| | prostate tumor, adenoCA, 65M, m/PROSNOT20 |
| | prostate, 32M, SUB, 3′ CGAP |
| | prostate, AH, mw/adenoCA, 66M |
| | seminal vesicle, aw/adenoCA, 61M |
| | sm intestine, 31F, RP |
| | sm intestine, 55F |
| | spinal cord, cervical, aw/lymphoma, 32M |
| | umb cord blood, T-lymphocytes, Th2 cells, untreated |
| | uterus, tumor line, adenoCA/leiomyoSAR, pool, MGC, EF |
| | colon, cecum, mw/Crohn's, 18F |
| | kidney tumor, clear cell type cancer, pool, SUB, CGAP |
| | Placenta, choriocarcinoma |
| #41528: | liver tumor line, C3A, Hepatob, 15M, Untx |
| | periph blood, promonocyte line, THP-1, AML, t/5AZA, SUB |
| | eye, retina, 9-80M/F, pool, AMP, EF |
| | periph blood, promonocyte line, THP-1, AML, untreated |
| | brain tumor, frontal, astrocytoma, 17F |
| | colon tumor, adenoCA, NORM, SUB, CGAP |
| | colon, descending, benign familial polyposis, 16M |
| | liver tumor line, C3A, Hepatob, 15M, t/MCA, SUB |
| | liver tumor line, C3A, Hepatob, 15M, t/MCA-48 hr |
| | liver tumor line, C3A, Hepatob, 15M, t/PB-48 hr |
| | lung tumor line, sm/lg cell CA/mucoepidermoid CA, pool, MGC, EF |
| | periph blood, monocytes, 42F, t/IL-10, LPS, NORM |
| | spleen, Gaucher's, 22M |
| | thymus, aw/patent ductus arteriosus, 3M, 5RP |
| | umb cord blood, mononuclear cells, t/IL-5 |
| | adrenal glands, aw/anencephaly, fetal, 16wF, lg cDNA |
| | bone tumor, sacrum, giant cell tumor, 18F |
| | bone, rib, aw/Patau's, fetal, 20wM, lg cDNA |
| | brain, infant, 10wF, NORM, WM |
| | brain, medulla, Huntington's, mw/CVA, 57M |
| | brain, pineal gland, CGAP/WN |
| | brain, pineal gland, aw/AD, 68, 79F, pool, NORM |
| | brain, pineal gland, aw/AD, COPD, 79F |
| | breast, PF changes, mw/multifocal ductal CA in situ, 46F |
| | breast, mw/ductal CA, CA in situ, aw/node mets, 62F |
| | colon tumor, hepatic flexure, adenoCA, 55M, m/COLATMT01 |
| | colon, ascending, CUC, 25F |
| | colon, ascending, mw/CUC, 28M |
| | colon, epithelium, 13F |
| | colon, ulcerative colitis, 16M |
| | eye tumor line, retinoblastoma, pool, MGC, EF |
| | eye, retina, 55M, NORM, WM |
| | eye, retina, 9-80M/F, pool, NORM, EF |
| | germ cell tumor, pool, SUB, 3′ CGAP |
| | heart, fetal, TIGR |
| | heart, left ventricle, Pompe's, 7mM |
| | kidney, pool, SUB, 3′ CGAP |
| | liver tumor line, C3A, Hepatob, 15M, t/APAP-48 hr |
| | liver, pool, NORM, EF |
| | liver, primary biliary cirrhosis, 63F, RP |
| | liver/spleen, fetal, 20wM, NORM, WM |
| | lung tumor, neuroendocrine carcinoid, pool, SUB, CGAP |
| | mixed tissues, fetal lung, testis, B-cell, SUB, 3′ CGAP/WN |
| | pancreas, pancreatitis, mw/adenoCA, 65F, m/PANCTUT01 |
| | periph blood, macrophages, adher PBMC, M/F |
| | periph blood, monocytes, 42F, t/antiIL-10, LPS, NORM |
| | periph blood, monocytes, 42F, t/antiIL-10, LPS, SUB |
| | placenta, aw/hydrocephalus, fetal, 16w, RP |
| | skin tumor line, melanoma/squamous cell CA, pool, MGC, EF |
| | spinal cord, cervical, aw/lymphoma, 32M |
| | spleen, splenomegaly, 59M, EF |
| | testis, M, NORM, CGAP/WN |
| | thymus, 3M, NORM |
| | thymus, aw/congenital heart abnormalities, 2F |
| | tonsil, B-lymphocytes, germinal, aw/tonsillitis, NORM, CGAP |
| | testis |
| #40893: | brain tumor, oligodendroglioma, NORM, CGAP |
| | brain, aw/hypoplastic left heart, fetal, 23wM |
| | brain, aw/hypoplastic left heart, fetal, 23wM, 5RP |
| | brain, medulla, aw/CHF, 35M, AMP |
| | brain, archaeocortex, hippocampus, aw/cholangioCA, 55F, RP |
| | brain, aw/hypoplastic left heart, fetal, 23wM, 5RP, EF |
| | brain, aw/hypoplastic left heart, fetal, 23wM, NORM |
| | brain, caudate/putamen/nucleus accumbens, aw/CHF, 35M |
| | brain, fetal, 23wM, 5C-RP |
| | nervous tissue, pool, LICR, EF |
| | ovary tumor, fibrothecoma, F, pool, NORM, 3′, CGAP |
| | brain line, fetal, 17-18wF, RP, 3′ TIGR |
| | brain, aw/hypoplastic heart, fetal, 23wM, pool, RP, EF |
| | brain, pineal gland, aw/AD, 68, 79F, pool, NORM |
| | mixed, brain/pituitary, Nrml/Huntington's/AD, M/F, pool, 5RP, EF |
| | kidney |
| | brain, hippocampus |
| | brain |
| #40994: | colon, aw/Patau's, fetal, 20wM, lg cDNA |
| | prostate tumor, CA, pool, M, LICR, EF |
| | Jurkat line, T-cell leukemia, M, t/PMA, Iono-1 hr, 5C-FL, EF |
| | bladder tumor line, CA/TC CA/papilloma, pool, MGC, EF |
| | bone tumor, rib, mets osteoSAR, 16M |
| | brain tumor, frontal, astrocytoma, 47M |
| | brain, astrocytes, fetal, 22wF, t/TNF, IL-1 24 hr |
| | brain, aw/hypoplastic left heart, fetal, 23wM, 5RP |
| | brain, cerebellum, Huntington's, mw/CVA, 57M, RP |
| | brain, choroid plexus, Huntington's, mw/CVA, 57M, RP |
| | brain, globus pallidus/substantia innominata, aw/CHF, 35M |
| | brain, hippocampus, aw/CHF, 35M |
| | brain, hypothalamus, Huntington's, mw/CVA, 57M |
| | breast tumor line, T-47D, ductal CA, 54F, Untx, NORM, EF |
| | breast tumor line, adenoCA, MGC, EF |
| | colon tumor line, KM12C, CA, Untx, TIGR |
| | epiglottis, aw/papillary thyroid CA, 71M |
| | esophagus tumor, adenoCA, 61M, NORM |
| | fat, abdomen, aw/obesity, 52F |
| | heart, coronary artery, CAD, 46M |
| | liver, primary biliary cirrhosis, 63F, RP |
| | liver/spleen, fetal, 20wM, NORM, CGAP/WM/WN |
| | lung, aw/anencephaly, fetal, 20wF |
| | lung, fetal, 23wM |
| | lung, mw/adenoCA, COPD, 47M |
| | muscle, skeletal, mw/malignant hyperthermia, WM/WN |
| | muscle, thigh, mw/lipoSAR, 58M, RP |
| | neuroganglion tumor, ganglioneuroma, 9M |
| | ovary, dermoid cyst/aw/leiomyoma, 22, 47F, pool, lg cDNA |
| | ovary, stromal hyperthecosis, mw/dermoid cyst, 45F, RP |
| | periph blood, macrophages, adher PBMC, M/F, MLR-24 hr |
| | placenta, aw/hydrocephalus, fetal demise, 16w, 18wM, pool |
| | prostate, 32M, NORM, 3′/5′ CGAP |
| | prostate, 32M, SUB, 3′ CGAP |
| | prostate, AH, mw/adenoCA, 55M, m/PROSTUT16 |
| | seminal vesicle, aw/prostate adenoCA, 67M |
| | spinal cord, base medulla, Huntington's, 57M, lg cDNA, EF |
| | stomach tumor, CA, pool, LICR, EF |
| | stomach, aw/esophagus adenoCA, 61M, 5RP |
| | testis, 16M |
| | tonsil, B-lymphocytes, germinal, aw/tonsillitis, NORM, CGAP |
| | umbilical vein, endothelial cells, pool, WM/WN |
| | uterus, endometrium, type II defect, endometriosis, F |
| | uterus, myometrium, mw/leiomyoma, 41F, RP, m/UTRSTUT05 |
| | uterus, tumor line, adenoCA/leiomyoSAR, pool, MGC, EF |
| | adrenal gland, aw/pituitary neoplasm, 61F |
| | adrenal gland, aw/renal cell CA, 43M |
| | aorta, 64M, WN |
| | bladder tumor, TC CA, 72M |
| | bladder, mixed, TC CA/Nrml, 58, 72M, pool, NORM, EF |
| | bone tumor, Ewing's SAR, CGAP |
| | brain tumor, frontal/parietal, meningioma, 76F |
| | brain tumor, glioblastoma, pool, NORM, CGAP |
| | brain tumor, mixed, pool, MGC, EF |
| | brain tumor, oligodendroglioma, 3′ CGAP |
| | brain, allocortex/neocortex, cingulate, aw/CA, 55F, RP |
| | brain, amygdala, aw/CHF, 35M |
| | brain, aw/muscular atrophy, 3mF, NORM, GEXP |
| | brain, caudate/putamen/nucleus accumbens, aw/CHF, 35M |
| | brain, cerebellum, Huntington's, mw/CVA, 57M |
| | brain, cerebellum, aw/COPD, 69M |
| | brain, cerebellum, aw/COPD, left ventricular failure, 70M |
| | brain, choroid plexus, Huntington's, mw/CVA, 57M |
| | brain, fetal, 15w, NORM, WM/WN |
| | brain, frontal cortex, aw/CHF, 35M |
| | brain, frontal cortex, aw/CHF, 35M, NORM |
| | brain, frontal cortex, aw/lung CA, 77M |
| | brain, frontal, Huntington's, mw/CVA, 57M |
| | brain, hypothalamus, Huntington's, mw/CVA, 57M, NORM |
| | brain, infant, 10wF, NORM, WM |
| | brain, medulla, Huntington's, mw/CVA, 57M |
| | brain, mixed tissues, aw/CHF, 35M, pool, lg cDNA |
| | brain, mixed tissues, aw/aortic aneurysm, 45F |
| | brain, multiple sclerosis |
| | brain, mw/oligoastrocytoma, epilepsy, 26M, NORM |
| | brain, mw/oligoastrocytoma, epilepsy, 26M, SUB |
| | brain, neocortex, temporal, aw/cholangioCA, 55F, RP |
| | brain, parietal cortex, aw/CHF, 35M |
| | brain, sensory-motor cortex, aw/CHF, 35M |
| | brain, striatum, caudate nucleus, schizophrenia, 49M |
| | brain, striatum/globus pallidus, AD, aw/mets adenoCA, 70F |
| | brain, striatum/globus pallidus/putamen, aw/CHF, 81F, RP |
| | brain, temporal, gliosis, mw/epilepsy, 27M, lg cDNA |
| | breast tumor, lobular CA, 58F, m/BRSTNOT05 |
| | breast, PF changes, mw/adenoCA, intraductal CA, 43F |
| | bronchial epithelium line, NHBE, 54M, Untx, NORM |
| | cervical tumor line, HeLa S3, adenoCA, 31F, untreated, WM/WN |
| | colon polyp, aw/adenoCA, tubulovillous adenoma, 40F |
| | colon tumor, adenoCA, NORM, 3′ CGAP |
| | colon tumor, adenoCA, NORM, SUB, CGAP |
| | colon tumor, adenoCA, carcinoid, M/F, pool, NORM |
| | colon tumor, adenoCA, pool, NORM, 3′ CGAP |
| | colon, cecum, Crohn's, 31M |
| | colon, mw/adenoCA, aw/node mets, 60M, m/COLNTUT16 |
| | eye tumor line, retinoblastoma, pool, MGC, EF |
| | eye, retina, 55M, NORM, WM |
| | fat, breast, aw/fibrosis, 38F |
| | ganglion, dorsal root, cervical, aw/lymphoma, 32M |
| | ganglion, dorsal root, thoracic, aw/lymphoma, 32M |
| | ganglion, dorsal root, thoracic, aw/lymphoma, 32M, NORM |
| | germ cell tumor, pool, 3′ CGAP |
| | germ cell tumor, pool, SUB, 3′ CGAP |
| | germ cell tumor, seminoma, teratoma adenoCA, pool, 3′ CGAP |
| | heart tumor, left atrium, myxoma, 43M |
| | heart, 44M, NORM |
| | heart, aorta, 39M, 5RP |
| | heart, aorta, aw/cerebral agenesis, 27F |
| | heart, coronary artery, plaque, pool |
| | heart, fetal, 8-10w, pool, BI |
| | heart, fetal, M |
| | heart, left atrium, 51F |
| | heart, left ventricle, 31M |
| | heart, left ventricle, mw/myocardial infarction, 56M |
| | hypothalamus, 16-75M/F |
| | iliac artery, endothelial cells, F, t/1% oxygen 24 hr |
| | kidney tumor line, CA/adenoCA/hypernephroma, pool, MGC, EF |
| | kidney tumor, Wilms', pool, WM/WN |
| | kidney tumor, clear cell type cancer, pool, NORM, 3′ CGAP |
| | kidney, 2dF |
| | kidney, mw/renal cell CA, 43M, m/KIDNTUT14 |
| | kidney, pool, NORM, 3′ CGAP |
| | kidney, pool, SUB, 3′ CGAP |
| | kidney, right/left, aw/Patau's, fetal, 20wM, pool, lg cDNA |
| | liver tumor, mets neuroendocrine CA, 62F, m/ LIVRTMR01 |
| | liver, MGC, EF |
| | liver, primary biliary cirrhosis, 63F |
| | liver/spleen, fetal, 20wM, NORM, WM |
| | lung fibroblast line, WI38, 3mF, untreated |
| | lung tumor, neuroendocrine carcinoid, pool, SUB, CGAP |
| | lung tumor, squamous cell CA, pooled, NORM, CGAP |
| | lung, 2M |
| | lung, 72M, WM/WN |
| | lung, MGC, EF |
| | lung, aw/Patau's, fetal, 20wM |
| | lung, fetal, 19 w, NORM, CGAP/WM/WN |
| | lung, fetal, TIGR |
| | lung, mw/endobronchial carcinoid, 33M |
| | lung, mw/mets osteoSAR, aw/pleura mets, 58M |
| | lymph node tumor, follicular lymphoma, 3′ CGAP |
| | lymph node tumor, mets melanoma, 3′, CGAP |
| | lymph nodes, 42F |
| | lymphocytes, PBMC, M, 96-hr MLR |
| | mixed tissues, fetal lung, testis, B-cell, SUB, 3′ CGAP/WN |
| | mixed tissues, includes tumor, SUB, 3′ CGAP |
| | mixed tumor, Wilm's/brain mets, pool, 3′ CGAP |
| | mixed, (melanocytes, uterus, fetal heart), SUB, CGAP/WM/WN |
| | multiple sclerosis, 46M, NORM, WM/WN |
| | muscle, arm, ALS, 74F |
| | muscle, gluteal, mw/clear cell SAR, 43F |
| | muscle, psoas, 12M |
| | muscle, skeletal, MGC, EF |
| | ovary tumor, TC CA, 53F |
| | ovary tumor, mixed types, pooled, F, 3′ CGAP |
| | pancreas tumor, adenoCA, 3′ CGAP |
| | pancreas, 8M |
| | pancreas, fetal, 23wM |
| | pancreas, pancreatitis, mw/adenoCA, 65F, m/PANCTUT01 |
| | parathyroid, mw/parathyroid CA, 44M |
| | penis, corpora cavernosa, M |
| | penis, corpus cavernosum, M |
| | periph blood, B-lymphocytes, CLL, pool, NORM, 3′ CGAP |
| | periph blood, eosinophils, hypereosinophilia, 48M |
| | periph blood, lymphocytes, non-adher PBMC, M/F, t/LPS |
| | periph blood, macrophages, adher PBMC, M/F, 48-hr MLR |
| | periph blood, promonocyte line, THP-1, AML, t/5AZA, SUB |
| | periph blood, promonocyte line, THP-1, AML, untreated |
| | peritoneum tumor, neuroendocrine CA, 66F |
| | placenta, MGC, EF |
| | placenta, fetal, 21wF |
| | placenta, fetal, 8-9w, pool, NORM, CGAP/WM |
| | prostate tumor line, LNCaP, CA, 50M, untreated |
| | prostate tumor, adenoCA, 58M |
| | prostate tumor, adenoCA, 61M |
| | prostate tumor, cancer, 45M, m/PROETMP01/02, CGAP |
| | prostate, 28M |
| | prostate, AH, mw/adenoCA, M, m/PROSTUT13, PROSTUS08/19/20/25 |
| | seminal vesicle, aw/adenoCA, 56M |
| | skin, breast and fetal, aw/intraductal CA, pool |
| | skin, breast, 26F |
| | skin, foreskin, melanocytes, M, NORM, WM/WN |
| | sm intestine, fetal, 20wF |
| | sm intestine, ileum, Crohn's, 18F |
| | stomach, fetal, 18wM |
| | testis, M, NORM, CGAP/WN |
| | thymus, aw/congenital heart abnormalities, 2F |
| | thymus, hyperplasia, aw/myasthenia gravis, 16F |
| | thyroid tumor, follicular adenoma, 17M |
| | thyroid, mw/follicular adenoma, 28F |
| | uterus tumor, endometrial adenoCA, F, pooled, 3′ CGAP |
| | uterus tumor, leiomyoma, 41F |
| | uterus tumor, leiomyoma, m/UTRSNON03, UTRSNOT12, UTRSTMR01 |
| | uterus, F, NORM, CGAP/WM/WN |
| | uterus, aw/ovarian follicular cysts, 34F |
| | uterus, endometrium, mw/cervicitis, leiomyoma, pool, lg cDNA |
| | uterus, myometrium, mw/adenoCA, 50F |
| | kidney |
| | PBMC, 60M, untreated |
| | adrenal glands, aw/anencephaly, fetal, 16wF, lg cDNA |
| | aorta, adventitia, 65F |
| | aortic smooth muscle line, M |
| | bladder tumor, TC CA, 58M, m/BLADNOT09 |
| | brain, acute/chronic multiple sclerosis, pool |
| | brain, aw/hypoplastic left heart, fetal, 23wM, 5RP, EF |
| | brain, aw/hypoplastic left heart, fetal, 23wM, NORM |
| | brain, frontal, gliosis, mw/epilepsy, cerebral palsy, 27M |
| | brain, frontal, mw/astrocytoma, 40F, m/BRAITUT12/ BRAFTUE03 |
| | brain, hippocampus, mw/intracranial hemorrhage, 72F, NORM |
| | brain, temporal, mw/neuroepithelial tumor, epilepsy, 45M |
| | breast, pool, LICR, EF |
| | cartilage, knee, chondrocytes, M/F, t/IL-1 |
| | colon tumor, adenoCA, 3′ CGAP |
| | colon tumor, adenoCA, 60M, m/COLNNOT07/08/09/11 |
| | colon tumor, ileocecum, Burkitt lymphoma, 29F, m/COLANOT03 |
| | colon, ascending, CUC, 25F |
| | colon, descending, benign familial polyposis, 16M |
| | colon, transverse, Crohn's, 26M |
| | heart, 65M |
| | heart, aorta, 12F |
| | heart, fetal, 19w, NORM, CGAP/WM/WN |
| | heart, hypoplastic left, fetal, 23wM |
| | liver tumor line, C3A, Hepatob, 15M, Untx |
| | liver, 29M |
| | liver, aw/anencephaly, fetal, 20wF |
| | lung, 47M |
| | lung, aw/Patau's, fetal, 20wM, lg cDNA |
| | lung, mw/adenoCA, aw/node, diaphragm mets, 63F |
| | lung, mw/mets thyroid CA, 79M, m/LUNGTUT02 |
| | lymph node, 11F |
| | lymphocytes, activated Th2 cells, 6-hr AB |
| | mesentery tumor, sigmoid, mets mixed-mullerian tumor, 61F |
| | mixed tissue tumor, head/neck, CA, pool, LICR, EF |
| | mixed tissues, includes tumor, M/F, pool, 5RP, EF |
| | mixed tissues, includes tumor, pool, SUB, CGAP |
| | nervous tissue, pool, LICR, EF |
| | nose, nasal polyps |
| | ovary tumor, mets colon adenoCA, 58F, NORM |
| | ovary tumor, serous CA, mets colon CA, 44F, 5RP, EF |
| | ovary, cystic, aw/cystadenoma, 34F |
| | periph blood, T-lymphocytes, CD8+, 63M, untreated |
| | periph blood, eosinophils, nonallergic, M/F |
| | periph blood, eosinophils, t/IL-5 |
| | pituitary gland, aw/schizophrenia, COPD, 55M, NORM |
| | pituitary, 16-70M/F, pool |
| | placenta, aw/hydrocephalus, fetal, 16w, RP |
| | placenta, neonatal, F, NORM, WM |
| | placenta, pool, LICR, EF |
| | prostate tumor, adenoCA, 59M, SUB, m/PROSNOT19 |
| | prostate tumor, adenoCA, 69M, m/PROSNOT07 |
| | prostate, BPH, mw/adenoCA, 70M, SUB |
| | prostate, BPH, mw/adenoCA, PIN, 59M |
| | seminal vesicle, aw/adenoCA, 58M |
| | skin, tuberculoid/lepromatous leprosy, M/F, pool, AMP |
| | spinal cord, cervical, aw/lymphoma, 32M |
| | spleen, aw/pancreas neuroendocrine CA, 65F |
| | teratoCA line, NT2, t/5AZA-3d, SUB |
| | testis, 26M |
| | thymus, aw/anencephaly, fetal, 17wF |
| | umb cord blood, T-lymphocytes, Th2 cells, untreated |
| | uterus, cervix, 40F |
| | uterus, endometrium, mw/leiomyoma aw/endometriosis, 48F |
| | uterus, myometrium, 43F |
| | whole blood, myeloid cells, CML, pool, 3′ CGAP |
| | bone, rib, aw/Patau's, fetal, 20wM |
| | clavicle, osteoblasts, 40M, untreated, lg cDNA, EF |
| | colon, aw/anencephaly, fetal, 20wF |
| | synovium, rheuA, 75M/56F, pool, NORM |
| | thyroid, lymphocytic thyroiditis, mw/papillary CA, 30F |
| | hepatoma (HepG2) |
| | Eye, retinoblastoma |
| | whole embryo, mainly body |
| | brain |
| | testis |
| | Lung, mucoepidermoid carcinoma |
| | spleen |
| #49476: | bone marrow line, SH-SY5Y, neuroblastoma, 4F, t/6-OHDA, 5RP, EF |
| | colon, mw/adenoCA, aw/node mets, 60M, m/COLNTUT16 |
| | heart, fetal, 8-10w, pool, BI |
| | kidney, aw/hypoplastic left heart, fetal, 23wM |
| | kidney, pool, SUB, 3′ CGAP |
| | microvascular, dermal, endothelial cells, 22F, t/bFGF, EF |
| | prostate tumor line, CA/adenoCA, pool, MGC, EF |
| | spinal cord, aw/renal failure, 71M |
| | uterus, mw/leiomyoma, aw/colon adenoCA, 45F |
| | uterus, myometrium, mw/leiomyoma, 41F, NORM, m/UTRSTUT05 |
| | liver tumor line, C3A, Hepatob, 15M, t/insulin-24 hr, 5C-FL, EF |
| | mixed, brain/pituitary, Nrml/Huntington's/AD, M/F, pool, 5RP, EF |
| | Uterus, leiomyosarcoma |
| | Prostate, adenocarcinoma. |
| #49904: | brain, aw/hypoplastic left heart, fetal, 23wM, 5RP, EF |
| | CML precursor line, K-562, 53F, t/5AZA-72 hr |
| | adrenal gland, aw/renal cell CA, 43M |
| | brain, mixed tissues, aw/aortic aneurysm, 45F |
| | brain, temporal, polymicrogyria, gliosis, 5M |
| | colon, aw/gastroparesis, 37F |
| | kidney, cortex, mw/renal cell CA, 65M, 5RP |
| | liver tumor, mets neuroendocrine CA, 72M, 5RP, EF |
| | lung, fetal, M, RP |
| | pituitary, 16-70M/F, pool |
| | bone marrow tumor, CA, pool, LICR, EF |
| | brain, aw/hypoplastic left heart, fetal, 23wM, 5RP |
| | brain, aw/hypoplastic left heart, fetal, 23wM, lg cDNA |
| | mixed tissue tumor, head/neck, CA, pool, LICR, EF |
| | PBMC, 28-57M/F, pool, Untx-24 hr, FL-EN, EF |
| | UCB, derived dendritic cells, pool, untreated/treated, NORM |
| | brain, mixed tissues, aw/cholangioCA, 55F, RP |
| | nervous tissue, pool, LICR, EF |
| | adrenal gland, mixed, Nrml/pheochromocytoma, pool, lg cDNA, EF |
| | prostate, AH, mw/adenoCA, 58M |
| | skin tumor line, melanoma/squamous cell CA, pool, MGC, EF |
| | teratoCA line, hNT2, t/RA |
| | Jurkat line, T-cell leukemia, M, Untx, 5C-FL, EF |
| | Jurkat line, T-cell leukemia, M, t/PMA-30min, 5C-FL, EF |
| | Jurkat line, T-cell leukemia, M, t/PMA-30min, NORM, EF |
| | T-B lymphoblast line, leukemia, untreated |
| | T-lymphocytes, activated, TIGR |
| | T-lymphocytes, allogenic, 40-50M, untreated |
| | UCB, derived dendritic cells, M |
| | adrenal gland, 8M |
| | adrenal tumor, pheochromocytoma, 43F, m/ADRENOT11 |
| | adrenal tumor, pheochromocytoma, 52F, 5RP |
| | adrenal tumor, pheochromocytoma, 57F |
| | astrocytes, M/F, t/cytokines 4-6 hr |
| | astrocytes, M/F, untreated |
| | bladder tumor, TC CA, 72M |
| | bone marrow, 16-70M/F |
| | brain tumor, benign meningioma, 35F |
| | brain tumor, benign meningioma, 35F, NORM |
| | brain tumor, frontal, astrocytoma, 47M |
| | brain tumor, frontal, mets hypernephroma, 58M |
| | brain tumor, frontal/parietal, meningioma, 76F |
| | brain tumor, mixed types, pool, CGAP |
| | brain, 55M, NORM, WM |
| | brain, acute/chronic multiple sclerosis, pool |
| | brain, amygdala, aw/CHF, 35M |
| | brain, aw/muscular atrophy, 3mF, NORM, GEXP |
| | brain, cerebellum, Huntington's, mw/CVA, 57M |
| | brain, fetal, 23wM, FL-EN, EF |
| | brain, fetal, 24wF, TIGR |
| | brain, frontal, gliosis, mw/epilepsy, cerebral palsy, 27M |
| | brain, globus pallidus/substantia innominata, aw/CHF, 35M |
| | brain, hippocampus, aw/CHF, 35M |
| | brain, hippocampus, mw/intracranial hemorrhage, 72F, NORM |
| | brain, hypothalamus, pool, CHGC, EF |
| | brain, infant, 10wF, NORM, WM |
| | brain, medulla, aw/CHF, 35M |
| | brain, mixed tissues, aw/CHF, 35M, pool, lg cDNA |
| | brain, mw/oligoastrocytoma, epilepsy, 26M |
| | brain, mw/oligoastrocytoma, epilepsy, 26M, NORM |
| | brain, parietal cortex, aw/CHF, 35M |
| | brain, sensory-motor cortex, aw/CHF, 35M |
| | brain, striatum, caudate nucleus, schizophrenia, 49M |
| | brain, temporal cortex, aw/CHF, 35M |
| | brain, temporal cortex, aw/aortic aneurysm, 45F |
| | brain, temporal, gliosis, mw/epilepsy, 27M, lg cDNA |
| | brain, temporal, mw/neuroepithelial tumor, epilepsy, 45M |
| | breast tumor line, T-47D, ductal CA, 54F, 5C-FL, EF |
| | breast tumor, adenoCA, 54F, m/BRSTNOT03 |
| | breast tumor, ductal, F, pool, NORM, 3′/5′ CGAP |
| | breast tumor, low vascular density, control, F |
| | breast, PF changes, 40F |
| | breast, PF changes, mw/ductal adenoCA, 54F, m/BRSTTUT02 |
| | breast, mw/ductal CA, CA in situ, aw/node mets, 62F |
| | breast, mw/neoplasm, 36F |
| | breast, pool, LICR, EF |
| | colon tumor, adenoCA, 3′ CGAP |
| | colon tumor, adenoCA, 3′, CGAP |
| | colon tumor, adenoCA, M/F, pool, 3′ CGAP |
| | colon tumor, adenoCA, carcinoid, M/F, pool, NORM |
| | colon tumor, cecum, adenoCA, 70F |
| | colon, 16M |
| | colon, CUC, 69M |
| | colon, aw/Patau's, fetal, 20wM, lg cDNA |
| | colon, cecum polyp, aw/adenoCA, 67F |
| | colon, cecum, benign familial polyposis, 16M |
| | colon, cecum, mw/Crohn's, 18F |
| | colon, mw/adenoCA, 55M, m/COLHTUT01, COLHTUS02 |
| | colon, ulcerative colitis, 16M |
| | coronary artery, smooth muscle cells, 3M, NORM |
| | fat, abdomen, aw/obesity, 52F |
| | fat, axillary, aw/breast adenoCA, 73F |
| | gallbladder, cholecystitis, cholelithiasis, 55F, 5RP |
| | germ cell tumor, pool, NORM, 3′ CGAP |
| | germ cell tumor, pool, SUB, 3′ CGAP |
| | germ cell tumor, yolk sac, 3′ CGAP |
| | heart, aorta, 39M, 5RP |
| | heart, coronary artery, CAD, 46M |
| | heart, coronary artery, plaque, pool |
| | heart, fetal, 18wM |
| | heart, fetal, 19w, NORM, CGAP/WM/WN |
| | heart, fetal, 8-10w, pool, BI |
| | kidney epithelial transf embryo line, 293-EBNA, Untx |
| | kidney epithelial transf embryo line, 293-EBNA, lg cDNA |
| | kidney epithelial transf embryo line, 293-EBNA, t/5AZA |
| | kidney tumor line, CA/adenoCA/hypernephroma, pool, MGC, EF |
| | kidney tumor, clear cell type cancer, pool, SUB, CGAP |
| | kidney tumor, renal cell, 3′ CGAP |
| | kidney, aw/anencephaly, fetal, 17wF |
| | kidney, mw/renal cell CA, 43M, m/KIDNTUT14 |
| | kidney, mw/renal cell CA, 65M, m/KIDNTUT15 |
| | kidney, pool, SUB, 3′ CGAP |
| | periph blood, liver, 49M |
| | lung tumor, CA, pool, LICR, EF |
| | lung tumor, adenoCA, 63M |
| | lung tumor, neuroendocrine carcinoid, pool, SUB, CGAP |
| | lung tumor, squamous CA, 50M |
| | lung tumor, squamous cell CA, 57M |
| | lung, 35F, 5RP |
| | lung, MGC, EF |
| | lung, asthma, 15M |
| | lung, fetal, 19w, NORM, CGAP/WM/WN |
| | lung, mw/endobronchial carcinoid, 33M |
| | lung, mw/mets osteoSAR, aw/pleura mets, 58M |
| | lung, mw/mets osteoSAR, aw/pleura mets, 58M, NORM |
| | lung, pool, LICR, EF |
| | lymph node tumor, axillary, Hodgkin's disease, 45M |
| | meniscus, tibial, aw/mets alveolar rhabdomyoSAR, 16M |
| | microvascular, dermal, endothelial cells, neonatal, M |
| | mixed tissues, fetal lung, testis, B-cell, SUB, 3′ CGAP/WN |
| | mixed tissues, includes tumor, M/F, pool, NORM, EF |
| | mixed tissues, includes tumor, pool, SUB, CGAP |
| | mixed tissues, includes tumor, treated cells, pool, NORM, EF |
| | mixed tumor, sarcoma, pool, 3′ CGAP |
| | multiple sclerosis, 46M, NORM, WM/WN |
| | muscle tumor, striated, alveolar rhabdomyoSAR, 3′/5′ CGAP |
| | muscle, forearm, mw/intramuscular hemangioma 38F |
| | neuroectodermal tumor, peripheral, pool, LICR, EF |
| | nose, nasal polyps |
| | nose, nasal polyps, aw/asthma, 78M, pool, NORM |
| | ovary tumor, endometrioid CA, 62F |
| | ovary tumor, mucinous cystadenoCA, 43F, m/OVARNOT03 |
| | ovary, aw/leiomyomata, 36F |
| | ovary, aw/menorrhagia, 47F, 5RP |
| | ovary, endometriosis, aw/leiomyomata, 39F |
| | periph blood, pancreas tumor, adenoCA, 3′ CGAP |
| | periph blood, pancreas, 29M |
| | penis, aw/Patau's, fetal, 20wM, lg cDNA, EF, EF |
| | penis, corpus cavernosum, M |
| | penis, corpus cavernosum, mw/CA, 53M |
| | periph blood, dendritic cells, t/TNF |
| | periph blood, macrophages, adher PBMC, M/F |
| | periph blood, macrophages, adher PBMC, M/F, 48-hr MLR |
| | periph blood, macrophages, adher PBMC, M/F, MLR-24 hr |
| | periph blood, monocytes, 42F, t/IL-10, LPS |
| | periph blood, monocytes, 42F, t/antiIL-10, LPS |
| | periph blood, monocytes, 42F, t/antiIL-10, LPS, SUB |
| | periph blood, promonocyte line, THP-1, AML, 1M, t/tuberculosis |
| | periph blood, promonocyte line, THP-1, AML, stimulated |
| | periph blood, promonocyte line, THP-1, AML, t/5AZA, SUB |
| | periph blood, promonocyte line, THP-1, AML, untreated |
| | periph blood, peritoneum tumor, neuroendocrine CA, 66F |
| | placenta, aw/hydrocephalus, fetal demise, 16w, 18wM, pool |
| | placenta, aw/hydrocephalus, fetal, 16w, RP |
| | placenta, fetal, 18wM |
| | placenta, neonatal, F, NORM, WM |
| | prostate, 32M, SUB, 3′ CGAP |
| | sm intestine tumor, ileum, carcinoid, 42M |
| | sm intestine, ileum, Crohn's, 26M |
| | smooth muscle tumor, leiomyoSAR, pool, NORM, 3′/5′ CGAP |
| | soft tissue tumor, spinal schwannoma, 35M |
| | spinal cord, aw/renal failure, 71M |
| | stomach tumor, adenoCA, poorly differentiated, 3′ CGAP |
| | stomach, pool, LICR, EF |
| | synovium, 75M |
| | synovium, wrist, rheuA, 62F |
| | teratoCA line, hNT2, t/RA + MI |
| | teratoCA line, hNT2, t/mouse leptin, 9cis RA-6d, lg cDNA |
| | testis, 16M |
| | testis, M, NORM, CGAP/WN |
| | testis, aw/cirrhosis, 37M |
| | testis, necrosis, 31M |
| | thymus, aw/congenital heart abnormalities, 2F |
| | thymus, aw/parathyroid adenoma, 21M |
| | thymus, hyperplasia, aw/myasthenia gravis, 16F |
| | thyroid, mw/adenomatous goiter with follicular adenoma, 18F |
| | thyroid, mw/follicular adenoma, 28F |
| | tonsil, B-lymphocytes, germinal, aw/tonsillitis, NORM, CGAP |
| | tonsil, lymphoid hyperplasia, 6M |
| | umb cord blood, mononuclear cells, t/IL-5 |
| | urogenital tumor, TC CA, pool, 3′ CGAP |
| | uterus tumor, endometrial adenoCA, F, pooled, 3′ CGAP |
| | uterus, endometrium, mw/leiomyoma aw/endometriosis, 48F |
| | uterus, myometrium, mw/adenoCA, 50F |
| | uterus, tumor line, adenoCA/leiomyoSAR, pool, MGC, EF |
| | white blood cells, 27F |
| | adrenal glands, aw/anencephaly, fetal, 16wF, lg cDNA |
| | brain, thalamus, aw/CHF, 35M, NORM |
| | breast, NF breast disease, 32F |
| | lung tumor, neuroendocrine carcinoid, pool, NORM, 3′ CGAP |
| | umb cord blood, mononuclear cells, M/F, t/G-CSF, lg cDNA |
| #38149: | testis |
| #698561: | CML precursor line, K-562, 53F, t/5AZA-72 hr |
| | aorta, 64M, WN |
| | bone marrow, 16-70M/F |
| | brain tumor, benign meningioma, 35F |
| | brain tumor, frontal, astrocytoma, 40F, m/BRAINOT14 |
| | brain tumor, frontal, mets hypernephroma, 58M, 5RP |
| | brain tumor, oligodendroglioma, NORM, CGAP |
| | brain tumor, posterior fossa, meningioma, 70M |
| | brain, archaeocortex, hippocampus, aw/cholangioCA, 55F, RP |
| | brain, aw/hypoplastic left heart, fetal, 23wM |
| | brain, aw/hypoplastic left heart, fetal, 23wM, 5RP, EF |
| | brain, aw/hypoplastic left heart, fetal, 23wM, FL-EN, EF |
| | brain, aw/hypoplastic left heart, fetal, 23wM, RP |
| | brain, caudate nucleus, mw/CVA, 92M |
| | brain, corpus callosum, AD, 74M |
| | brain, frontal cortex, aw/CHF, 35M, NORM |
| | brain, hippocampus, 2F, SUB, 3′ TIGR |
| | brain, hippocampus, AD |
| | brain, hippocampus, aw/CHF, 35M, NORM |
| | brain, infant, 10wF, NORM, WM |
| | brain, mixed tissues, aw/aortic aneurysm, 45F |
| | brain, mixed tissues, aw/cholangioCA, 55F, RP |
| | brain, pineal gland, aw/AD, 68, 79F, pool, NORM |
| | brain, pons, AD, 74M |
| | brain, temporal, gliosis, mw/epilepsy, cerebral palsy, 27M |
| | brain, thalamus, aw/CHF, 35M, NORM |
| | breast tumor, adenoCA, 45F, m/BRSTNOT09 |
| | breast, PF changes, mw/adenoCA, intraductal CA, 43F |
| | colon tumor, adenoCA, NORM, 3′ CGAP |
| | colon tumor, adenoCA, carcinoid, M/F, pool, NORM |
| | colon tumor, ileocecum, Burkitt lymphoma, 29F, m/COLANOT03 |
| | colon, cecum, Crohn's, 31M |
| | colon, mixed tissues, 16M/13F, pool, NORM |
| | colon, mw/adenoCA, aw/node mets, 60M, m/COLNTUT16 |
| | colon, ulcerative colitis, 16M |
| | fallopian tube tumor, endometrioid/serous adenoCA, 85F, RP |
| | gallbladder tumor, squamous cell CA, 78F |
| | ganglion, dorsal root, cervical, aw/lymphoma, 32M, NORM, EF |
| | germ cell tumor, pool, SUB, 3′ CGAP |
| | kidney, aw/hypoplastic left heart, fetal, 23wM |
| | kidney, interstitial nephritis, aw/bladder TC CA, 63M, 5RP |
| | kidney, mw/benign cyst, nephrolithiasis, 42F |
| | liver tumor line, C3A, Hepatob, 15M, Untx, NORM |
| | liver/spleen, fetal, 20wM, NORM, CGAP/WM/WN |
| | lung, 2M |
| | lung, asthma, 17M |
| | lung, fetal, 19w, NORM, CGAP/WM/WN |
| | lung, panacinar emphysema, mw/granuloma, 61M |
| | lymph node, 11F |
| | lymph node, necrotic, aw/lung squamous cell CA, 67M |
| | mammary, epithelial cells, 21F, untreated |
| | mesentery tumor, sigmoid, mets mixed-mullerian tumor, 61F |
| | ovary, aw/leiomyomata, 36F |
| | ovary, mw/mucinous cystadenoCA, 43F, m/OVARTUT01 |
| | ovary, stromal hyperthecosis, mw/dermoid cyst, 45F, RP |
| | pancreas tumor, adenoCA, 3′ CGAP |
| | pancreas, 17F, NORM |
| | pancreatic tumor line, adenoCA, untreated, WM/WN |
| | pancreatic tumor, adenoCA, 65F, m/PANCNOT08 |
| | periph blood, eosinophils, hypereosinophilia, 48M |
| | periph blood, macrophages, adher PBMC, M/F, 48-hr MLR |
| | pituitary gland, aw/schizophrenia, COPD, 55M, NORM |
| | prostate tumor, adenoCA, 59M, SUB, m/PROSNOT19 |
| | prostate, AH, mw/adenoCA, 66M, m/PROSTUT17 |
| | skin, foreskin, melanocytes, M, NORM, WM/WN |
| | sm intestine, fetal, M, RP, EF |
| | stomach, aw/esophagus adenoCA, 61M, 5RP |
| | synovium, wrist, rheuA, 62F |
| | thymus, aw/congenital heart abnormalities, 2F, RP |
| | thyroid, mw/medullary, papillary CA, node mets, 56M |
| | uterus tumor, endometrial adenoCA, F, pooled, 3′ CGAP |
| | uterus, endometrium, mw/cervical dysplasia, 32F, 5RP |
| | bladder, 11M |
| | ganglion, dorsal root, cervical, aw/lymphoma, 32M |
| | kidney, right/left, aw/Patau's, fetal, 20wM, pool, lg cDNA |
| | brain tumor line, DU 145, mets prostate CA, 69M, Untx, 5C-FL, EF |
| | brain, striatum/globus pallidus/putamen, aw/CHF, 81F, RP |
| | lung, fetal, M, RP |
| | Jurkat line, Frac Nuc, T-cell leukemia, M, t/antiCD3, 5C-FL, EF |
| | T-lymphocytes, CD4+, pool, t/anti-CD3, anti-CD28 antibodies |
| | adrenal gland, mixed, Nrml/pheochromocytoma, pool, lg cDNA, EF |
| | bone marrow, tibia, aw/mets alveolar rhabdomyoSAR, 16M, EF |
| | bone tumor, rib, mets osteoSAR, 16M |
| | bone, rib, aw/Patau's, fetal, 20wM |
| | brain tumor, frontal, astrocytoma, 17F |
| | brain tumor, frontal, neuronal neoplasm, 32M |
| | brain, allocortex, cingulate, aw/cholangioCA, 55F, RP |
| | brain, amygdala/entorhinal cortex, aw/CA, 55F, RP, EF |
| | brain, aw/hypoplastic left heart, fetal, 23wM, lg cDNA |
| | brain, aw/muscular atrophy, 3mF, NORM, GEXP |
| | brain, aw/spinal muscular atrophy, 72dF, 3′ SIK/TIGR |
| | brain, cerebellum, aw/COPD, 69M |
| | brain, medulla, Huntington's, mw/CVA, 57M |
| | brain, substantia nigra, aw/atherosclerosis, CHF, 81F, NORM |
| | breast tumor, adenoCA, 55F, m/BRSTNOT02 |
| | breast, mw/lobular CA, 58F, m/BRSTTUT03 |
| | colon tumor, adenoCA, NORM, SUB, CGAP |
| | colon, descending, CUC, 28M, 5RP |
| | heart, 44M, NORM |
| | heart, aorta, 17F |
| | heart, left ventricle, Pompe's, 7mM, RP |
| | kidney epithelial transf embryo line, 293-EBNA, Untx |
| | kidney, 8M |
| | kidney, mw/renal cell CA, 8, 53F, pool, NORM |
| | kidney, pool, SUB, 3′ CGAP |
| | liver tumor line, C3A, Hepatob, 15M, t/MCA-48 hr, SUB |
| | liver, aw/Patau's, anencephaly, fetal, pool, lg cDNA |
| | lung, 35F, 5RP |
| | lung, fetal, 23wM |
| | lung, mw/adenoCA, aw/node, diaphragm mets, 63F |
| | lung, mw/mets osteoSAR, aw/pleura mets, 58M |
| | lymph node, peripancreatic, aw/pancreatic adenoCA, 65M |
| | mixed tissues, includes tumor, SUB, 3′ CGAP |
| | mixed tissues, includes tumor, pool, SUB, 3′ CGAP |
| | mixed tissues, includes tumor, treated cells, pool, NORM, EF |
| | muscle, gluteal, mw/clear cell SAR, 43F |
| | ovary tumor, mucinous cystadenoma, 51F |
| | ovary tumor, papillary serous cystadenoCA 36F, NORM, WM/WN |
| | penis, corpora cavernosa, aw/scrotal urothelial CA, M |
| | periph blood, macrophages, adher PBMC, M/F, t/LPS |
| | periph blood, monocytes, 42F, t/antiIL-10, LPS, SUB |
| | periph blood, promonocyte line, THP-1, AML, untreated |
| | prostate stroma, fibroblasts, fetal, 26wM, untreated, AMP |
| | prostate, AH, mw/adenoCA, 57M, m/PROSTUT04 |
| | renal vein, smooth muscle cells, 57M, Untx, 5RP, EF |
| | skin, breast, aw/adenoCA, 70F, lg cDNA |
| | sm intestine, 31F, RP |
| | spleen tumor, Hodgkin's, 45M |
| | spleen, Gaucher's, 22M |
| | stomach tumor, adenoCA, poorly differentiated, 3′ CGAP |
| | stomach, fetal, 20wF |
| | synovium, knee, OA, 82F |
| | teratoCA line, NT2, t/cytokines, 5C-FL, EF |
| | testis, aw/cirrhosis, 37M |
| | thymus, aw/congenital heart abnormalities, 2F |
| | thymus, aw/patent ductus arteriosus, 3M |
| | thymus, hyperplasia, aw/myasthenia gravis, 16F |
| | tonsil, B-lymphocytes, germinal, aw/tonsillitis, NORM, CGAP |
| | tonsil, lymphoid hyperplasia, 6M/9F, pool, lg cDNA, EF |
| | umb cord blood, T-lymphocytes, Th2 cells, untreated |
| | uterus tumor, serous papillary CA, F, pooled, 3′ CGAP |
| | uterus, cervix, F, NORM, 3′ CGAP |
| | uterus, myometrium, mw/adenoCA, 50F |
| | primary human renal epithelial cells |
| | Eye, normal, pigmented retinal epithelium |
| | hepatoma (HepG2) |
| | signet-ring cell carcinoma (KATO III) |
| | brain |
| | Skin, melanotic melanoma, high MDR. |
| | testis |
| #730440: | breast tumor, adenoCA, 55F, m/BRSTNOT02 |
| | colon tumor, adenoCA, carcinoid, M/F, pool, NORM |
| | heart, fetal, 19w, NORM, CGAP/WM/WN |
| | kidney epithelial transf embryo line, 293-EBNA, t/5AZA, SUB |
| | liver tumor, hepatoma, 50M |
| | osteogenic tumor line, Saos-2, SAR, 11F, untreated |
| | periph blood, promonocyte line, THP-1, AML, t/5AZA, SUB |
| | prostate tumor, adenoCA, 59M, SUB, m/PROSNOT19 |
| | CML precursor line, K-562, 53F, Untx |
| | CML precursor line, K-562, 53F, t/5AZA-72 hr |
| | Jurkat line, T-cell leukemia, M, untreated |
| | Supt line, T-lymphoblasts, leukemia, 8M, untreated, TIGR |
| | T-lymphocytes, activated, TIGR |
| | T-lymphocytes, allogenic anergic, 40-50M, t/OKT3 3 day |
| | T-lymphocytes, allogenic, 40-50M, t/OKT3 6 hr |
| | UCB, derived dendritic cells, M, t/PMA, Iono-5 hr |
| | UCB, derived dendritic cells, pool, untreated/treated, NORM |
| | adenoid, inflamed, 3 |
| | adrenal tumor, adenoma, pool, 3′ CGAP |
| | adrenal tumor, adrenal cortical CA, 49M, 5RP, EF |
| | aorta, endothelial cells, M |
| | bladder tumor, TC CA, 66M, m/BLADNOT06 |
| | bone marrow tumor line, SH-SY5Y, neuroblastoma, 4F, t/6-OHDA |
| | bone marrow tumor line, SH-SY5Y, pool, Untx/t/6OHDA, NORM, EF |
| | bone marrow, 16-70 M/F |
| | bone tumor line, MG-63, osteoSar, 14M, untreated |
| | bone tumor, Ewing's SAR, CGAP |
| | bone tumor, sacrum, giant cell tumor, 18F |
| | brain stem, aw/DMt1, 72M, NORM |
| | brain tumor, anaplastic oligodendroglioma, pool, NORM, CGAP |
| | brain tumor, frontal, meningioma, 61F |
| | brain, archaeocortex, hippocampus, aw/cholangioCA, 55F, RP |
| | brain, aw/hypoplastic left heart, fetal, 23wM |
| | brain, aw/hypoplastic left heart, fetal, 23wM, NORM |
| | brain, aw/muscular atrophy, 3mF, NORM, GEXP |
| | brain, aw/spinal muscular atrophy, 72dF, 3′ SIK/TIGR |
| | brain, cerebellum, TIGR |
| | brain, dentate nucleus, aw/CHF, 35M |
| | brain, fetal, 23wM |
| | brain, frontal cortex, aw/CHF, 35M |
| | brain, frontal, polymicrogyria, gliosis, 5M |
| | brain, globus pallidus/substantia innominata, aw/CHF, 35M |
| | brain, hippocampus, mw/intracranial hemorrhage, 72F, NORM |
| | brain, infant, 10wF, NORM, WM |
| | brain, pineal gland, aw/AD, 68, 79F, pool, NORM |
| | brain, pineal gland, aw/AD, CHF, DM, 68F |
| | brain, pons, AD, 74M |
| | brain, pons, aw/CHF, 35M |
| | brain, temporal, mw/mets malignant melanoma, 34M |
| | brain, temporal, mw/neuroepithelial tumor, epilepsy, 45M |
| | brain, thalamus, aw/CHF, 35M, NORM |
| | breast tumor line, T47D, ductal CA, 54F |
| | breast tumor, ductal CA, 43F, m/BRSTTMT01 |
| | breast tumor, ductal CA, 65F |
| | breast tumor, ductal, F, pool, NORM, 3′/5′ CGAP |
| | breast tumor, lobular CA, 58F, m/BRSTNOT05 |
| | breast, 46, 60F, pool, NORM |
| | breast, NF breast disease, 32F |
| | breast, NF changes, mw/ductal adenoCA, 40-57F, pool, lg cDNA |
| | breast, mw/ductal CA, CA in situ, aw/node mets, 62F |
| | breast, mw/ductal adenoCA, 46F, m/BRSTTUS08, BRSTTUT13 |
| | breast, mw/ductal adenoCA, CA in situ, 62F, m/BRSTTUT14 |
| | breast, mw/ductal adenoCA, intraductal CA, aw/node mets, 57F |
| | bronchus, 15M |
| | cervical tumor line, HeLa, adenoCA, 31F, t/5AZA 72 hr |
| | cervical tumor line, HeLa, adenoCA, 31F, t/TNF, IL-1 |
| | cervical tumor line, HeLa, adenoCA, 31F, untreated |
| | colon tumor, adenoCA, 3′, CGAP |
| | colon tumor, adenoCA, 75M, m/COLNNOT01 |
| | colon tumor, adenoCA, NORM, 3′ CGAP |
| | colon tumor, adenoCA, pool, 3′ CGAP |
| | colon tumor, adenoCA, pool, NORM, 3′/5′ CGAP |
| | colon, ascending, CUC, 32M |
| | colon, aw/Patau's, fetal, 20wM, lg cDNA |
| | colon, aw/anencephaly, fetal, 20wF |
| | colon, descending, benign familial polyposis, 16M |
| | colon, mw/adenoCA, 55M, m/COLHTUT01, COLHTUS02 |
| | colon, sigmoid, mw/adenoCA, aw/node mets, 62M, m/COLNTUT03 |
| | colon, transverse, benign familial polyposis, 16M |
| | fallopian tube tumor, endometrioid/serous adenoCA, 85F |
| | fat, mesentery, aw/diverticulosis, diverticulitis, 71M |
| | fetus, 8-9w, pool, NORM, CGAP/WM/WN |
| | gallbladder, cholecystitis, cholelithiasis, 18F |
| | gallbladder, cholecystitis, cholelithiasis, 21M |
| | ganglion, dorsal root, cervical, aw/lymphoma, 32M |
| | ganglion, dorsal root, cervical, aw/lymphoma, 32M, NORM, EF |
| | germ cell tumor, pool, NORM, 3′ CGAP |
| | heart, aorta, 10M |
| | heart, aorta, 17F |
| | heart, fetal, 8-10w, pool, BI |
| | heart, fetal, M |
| | heart, left ventricle, Pompe's, 7mM |
| | kidney epithelial transf embryo line, 293-EBNA, Untx |
| | kidney epithelial transf embryo line, 293-EBNA, serum starv |
| | kidney epithelial transf embryo line, 293-EBNA, t/5AZA |
| | kidney epithelial transf embryo line, 293-EBNA, tf/bgal |
| | kidney tumor, Wilms', pool, WM/WN |
| | kidney tumor, renal cell CA, 43M, m/KIDNNOT20 |
| | kidney tumor, renal cell CA, pool, 3′/5′ CGAP |
| | kidney, pool, NORM, 3′ CGAP |
| | liver tumor line, C3A, Hepatob, 15M, t/MCA, SUB |
| | liver tumor line, C3A, Hepatob, 15M, t/PB-48 hr |
| | liver, aw/Patau's syndrome, fetal, 20wM, 5RP |
| | liver, aw/anencephaly, fetal, 16wF |
| | liver, fetal, M, 5RP |
| | liver, pool, NORM, EF |
| | liver, primary biliary cirrhosis, 63F, RP |
| | liver/spleen, fetal, 20wM, NORM, CGAP/WM/WN |
| | liver/spleen, fetal, 20wM, NORM, WM |
| | lung tumor, mets granulosa cell tumor, 80F |
| | lung tumor, squamous cell CA, 50M |
| | lung tumor, squamous cell CA, 65F |
| | lung tumor, squamous cell CA, 68M |
| | lung tumor, squamous cell CA, 69M, m/LUNGNOT15 |
| | lung, 12M |
| | lung, 35F, 5RP |
| | lung, NORM |
| | lung, aw/anencephaly, fetal, 17wF |
| | lung, fetal, 19w, NORM, CGAP/WM/WN |
| | lung, mw/caseating granuloma, 58F |
| | lung, mw/endobronchial carcinoid, 33M |
| | lung, mw/mets osteoSAR, aw/pleura mets, 58M |
| | lung, right bronchus, nonasthmatic, 18-55M/F, pool |
| | lymph node, 16mM |
| | lymph node, necrotic, aw/lung squamous cell CA, 67M |
| | lymph nodes, 16mM, NORM |
| | lymphocytes, PBMC, M, 96-hr MLR |
| | mammary, epithelial cells, 21F, untreated, NORM |
| | meniscus, tibial, aw/mets alveolar rhabdomyoSAR, 16M |
| | microvascular, dermal, endothelial cells, 18F, untreated |
| | microvascular, dermal, endothelial cells, 30F, untreated |
| | mixed tissues, fetal lung, testis, B-cell, SUB, 3′ CGAP/WN |
| | mixed tissues, includes tumor, 16w-85M/F, pool, 5RP, EF |
| | mixed tissues, includes tumor, Untx cells, M, pool, NORM, EF |
| | mixed tissues, includes tumor, treated cells, pool, NORM, EF |
| | mixed tumor, Wilm's/brain mets, pool, 3′ CGAP |
| | mixed tumor, breast/mets ovary, F, pool, 3′, CGAP |
| | mixed, (melanocytes, uterus, fetal heart), SUB, CGAP/WM/WN |
| | muscle, forearm, mw/intramuscular hemangioma 38F |
| | muscle, glossal, mw/squamous cell CA, 41F |
| | neural tumor, schwannoma, pool, 3′ CGAP |
| | neuroganglion tumor, ganglioneuroma, 9M |
| | ovary tumor, mixed types, pooled, F, 3′ CGAP |
| | ovary tumor, papillary serous cystadenoCA 36F, NORM, WM/WN |
| | ovary tumor, seroanaplastic CA, 52F |
| | ovary, aw/leiomyomata, 36F, NORM |
| | ovary, endometriosis, aw/leiomyomata, 39F, NORM, EF |
| | pancreatic tumor line, adenoCA, untreated, WM/WN |
| | paraganglion tumor, paraganglioma, aw/renal cell CA, 46M |
| | parathyroid tumor, adenoma, M/F, NORM, WM |
| | penis, corpora cavernosa, M |
| | periph blood, eosinophils, nonallergic, M/F |
| | periph blood, promonocyte line, THP-1, AML, control |
| | periph blood, promonocyte line, THP-1, AML, t/5AZA |
| | periph blood, promonocyte line, THP-1, AML, untreated |
| | peritoneum tumor, neuroendocrine CA, 66F |
| | pituitary gland, aw/schizophrenia, COPD, 55M, NORM |
| | placenta, aw/hydrocephalus, fetal, 16w, 5RP |
| | probable astrocytes, M/F, untreated |
| | prostate tumor, adenoCA, 57M, m/PROSNOT06 |
| | prostate tumor, adenoCA, 59M, m/PROSNOT19 |
| | prostate tumor, adenoCA, M, m/PROSNOT28/PROSTMC01/PROSTMT05 |
| | prostate tumor, cancer, 45M, m/PROETMP01/02, CGAP |
| | prostate, 21M, TIGR |
| | prostate, 28M |
| | prostate, AH, mw/adenoCA, 57M, m/PROSTUT04 |
| | prostate, AH, mw/adenoCA, 58M |
| | prostate, AH, mw/adenoCA, 66M |
| | prostate, AH, mw/adenoCA, 68M |
| | prostate, AH, mw/adenoCA, M, m/PROSTUT13, PROSTUS08/19/20/25 |
| | prostate, AH, mw/adenoCA, node mets, 55M, lg/N, m/PROSTUT16 |
| | prostate, epithelial cells, 17M, untreated, NORM |
| | prostate, epithelium, mw/cancer, PIN, 45M, m/PROETUP02, CGAP |
| | prostate, mw/adenoCA, 68M, m/PROSTUT18 |
| | renal vein, smooth muscle cells, 57M t/TNF, IL1 |
| | seminal vesicle, aw/adenoCA, 56M |
| | sm intestine tumor, ileum, carcinoid, 42M |
| | sm intestine tumor, ileum, mets ovarian adenoCA, 49F |
| | sm intestine, 13M |
| | sm intestine, 31F, RP |
| | sm intestine, aw/Patau's syndrome, fetal, 20wM, 5RP |
| | sm intestine, fetal, 23wM, FL-EN, EF |
| | sm intestine, fetal, M, RP, EF |
| | soft tissue tumor, spinal schwannoma, 35M |
| | spinal cord, cervical, aw/lymphoma, 32M |
| | spleen tumor, malignant lymphoma, 28M, 5RP |
| | spleen, 2M |
| | spleen, fetal, WM |
| | spleen, fetal, pool |
| | stomach tumor, adenoCA, poorly differentiated, 3′ CGAP |
| | stomach, aw/esophagus adenoCA, 23w-61M, pool, AMP |
| | synovium, 75M, lg cDNA, EF |
| | teratoCA line, NT2, Untx, NORM |
| | teratoCA line, NT2, t/5AZA-3d |
| | teratoCA line, NT2, t/5AZA-3d, SUB |
| | teratoCA line, hNT2, untreated, WM/WN |
| | testis, 16M |
| | testis, M, NORM, CGAP/WN |
| | thymus, 3M |
| | thymus, aw/congenital heart abnormalities, 2F |
| | thymus, aw/congenital heart abnormalities, 2F, RP |
| | thymus, aw/patent ductus arteriosus, 3M |
| | thymus, aw/patent ductus arteriosus, 3M, 5RP |
| | thyroid tumor, follicular adenoma, 17M |
| | thyroid, AH, mw/papillary CA, 22F, 5RP, EF |
| | thyroid, mw/medullary, papillary CA, node mets, 56M |
| | umb cord blood, T-lymphocytes, Th2 cells, untreated |
| | uterus tumor, endometrial adenoCA, F, pooled, 3′ CGAP |
| | uterus tumor, endometrium, adenosquamous CA, 49F, 5RP |
| | uterus tumor, leiomyoma, m/UTRSNON03, UTRSNOT12, UTRSTMR01 |
| | uterus tumor, serous papillary CA, F, pooled, 3′ CGAP |
| | uterus, cervix tumor, CA, F, NORM, 3′ CGAP |
| | uterus, endometrium, F, pool |
| | uterus, endometrium, mw/cervical dysplasia, 32F |
| | uterus, endometrium, mw/endometrial polyp, 35F |
| | uterus, endometrium, mw/leiomyoma aw/endometriosis, 48F |
| | uterus, endometrium, type I defect, 30, 32, 36F, pool |
| | uterus, mixed tissues, mw/tumor, F, pool, lg cDNA, EF |
| | uterus, myometrium, mw/leiomyoma, 41F, RP, m/UTRSTUT05 |
| | gallbladder, cholecystitis, cholelithiasis, 55F, 5RP |
| | skin, leg, keratinocytes, neonatal, M |
| | bone marrow tumor line, SH-SY5Y, neuroblastoma, 4F, t/RA |
| | brain, parietal cortex, aw/CHF, 35M |
| | brain, sensory-motor cortex, aw/CHF, 35M |
| | breast tumor, adenoCA, 46F, m/BRSTNOT33, EF |
| | sm intestine, duodenum, 16M |
| | uterus tumor, leiomyomata/adenosquamousCA, F, pool, lg cDNA, EF |
| | brain, hippocampus, aw/CHF, 35M, NORM |
| | ovary, epithelial cell line, pool, F, 3′, CGAP |
| | periph blood, B-lymphocytes, CLL, pool, NORM, 3′ CGAP |
| | prostate tumor, cancer, M, pool, 3′ CGAP |
| | prostate, AH, mw/adenoCA, 55M, m/PROSTUT16 |
| | spinal cord, aw/renal failure, 71M |
| | breast cancer(MDA-MB453) |
| | Lung, small cell carcinoma |
| #51594: | brain, fetal, 23wM, 5C-RP |
| | adrenal gland, 20M, NORM |
| | brain, hippocampus, AD |
| | brain, hippocampus, aw/CHF, 35M |
| | brain, infant, 10wF, NORM, WM |
| | brain, infant, F, TIGR |
| | brain, mixed tissues, aw/aortic aneurysm, 45F |
| | brain, pineal gland, aw/AD, 68, 79F, pool, NORM |
| | brain, sensory-motor cortex, aw/CHF, 35M |
| | breast tumor, ductal CA, 65F |
| | breast tumor, ductal adenoCA, 66F |
| | colon, descending, benign familial polyposis, 16M |
| | colon, epithelium, 13F, RP |
| | fetus, 8-9w, pool, NORM, CGAP/WM/WN |
| | ganglion, dorsal root, thoracic/lumbar, aw/lymphoma, 32M |
| | hypothalamus, 16-75M/F |
| | kidney, cortex, mw/renal cell CA, 65M |
| | kidney, cortex, mw/renal cell CA, 65M, 5RP |
| | kidney, fetal, 19-23w, M/F, lg cDNA |
| | lung, mw/mets osteoSAR, aw/pleura mets, 58M, NORM |
| | pancreas, 8M, 5RP |
| | parathyroid, mw/parathyroid CA, 44M |
| | pituitary gland, aw/schizophrenia, COPD, 55M, NORM |
| | placenta, neonatal, F, NORM, WM |
| | prostate, AH, mw/adenoCA, M, m/PROSTUT13, PROSTUS08/19/20/25 |
| | prostate, AH, mw/adenoCA, node mets, 55M, lg/N, m/PROSTUT16 |
| | sm intestine, aw/stomach ulcer, 49F, 5RP |
| | thymus, 3M, NORM |
| | thymus, aw/congenital heart abnormalities, 2F, RP |
| | thyroid, mw/follicular adenoma, 28F |
| | uterus, endometrium, mw/leiomyoma aw/endometriosis, 48F |
| | brain tumor, frontal, mets hypernephroma, 58M, 5RP |
| | brain, aw/hypoplastic left heart, fetal, 23wM, 5RP |
| | brain, cerebellum, Huntington's, mw/CVA, 57M, RP |
| | brain, hippocampus, aw/atherosclerosis, CHF, 81F |
| | testis tumor, embryonal CA, 31M, EF |
| | uterus, aw/liver & breast cancer, 46F |
| | CML precursor line, K-562, 53F, Untx |
| | adrenal glands, aw/anencephaly, fetal, 16wF, lg cDNA |
| | adrenal tumor, pheochromocytoma, 43F, m/ADRENOT11 |
| | bladder and seminal vesicle, 28M |
| | brain line, fetal, 17-18wF, RP, 3′ TIGR |
| | brain tumor, anaplastic oligodendroglioma, pool, NORM, CGAP |
| | brain tumor, benign meningioma, 35F |
| | brain tumor, frontal, mets hypernephroma, 58M |
| | brain tumor, mixed types, pool, CGAP |
| | brain, 55M, NORM, WM |
| | brain, allocortex, cingulate, aw/cholangioCA, 55F, RP |
| | brain, allocortex/neocortex, cingulate, aw/CA, 55F, RP |
| | brain, amygdala, aw/CHF, 35M |
| | brain, aw/hypoplastic left heart, fetal, 23wM |
| | brain, aw/hypoplastic left heart, fetal, 23wM, 5C-RP |
| | brain, aw/hypoplastic left heart, fetal, 23wM, NORM |
| | brain, aw/hypoplastic left heart, fetal, 23wM, RP |
| | brain, aw/hypoplastic left heart, fetal, 23wM, lg cDNA |
| | brain, aw/muscular atrophy, 3mF, NORM, GEXP |
| | brain, caudate/putamen/nucleus accumbens, aw/CHF, 35M |
| | brain, cerebellum, AD, 74M |
| | brain, cerebellum, Huntington's, mw/CVA, 57M |
| | brain, cerebellum, aw/COPD, 69M |
| | brain, cerebellum, aw/bronchial CA, 64M |
| | brain, corpus callosum, AD, 74M |
| | brain, dentate nucleus, aw/cholangioCA, 55F, RP |
| | brain, fetal, 17w, SIK |
| | brain, fetal, 23wM |
| | brain, frontal cortex, aw/CHF, 35M, NORM |
| | brain, frontal, Huntington's, mw/CVA, 57M |
| | brain, frontal, gliosis, mw/epilepsy, cerebral palsy, 27M |
| | brain, globus pallidus/substantia innominata, aw/CHF, 35M |
| | brain, hippocampus, aw/aortic aneurysm, 45F, 5RP |
| | brain, hypothalamus, Huntington's, mw/CVA, 57M, NORM |
| | brain, medulla, aw/CHF, 35M |
| | brain, mixed tissues, aw/CHF, 35M, pool, lg cDNA |
| | brain, mixed tissues, aw/CHF, 35M, pool, lg/N |
| | brain, mixed, archaecortex/hippocampus, 55F, pool, AMP/N |
| | brain, multiple sclerosis |
| | brain, mw/oligoastrocytoma, epilepsy, 26M |
| | brain, mw/oligoastrocytoma, epilepsy, 26M, NORM |
| | brain, mw/oligoastrocytoma, epilepsy, 26M, SUB |
| | brain, neocortex, frontal, aw/cholangioCA, 55F, RP |
| | brain, neocortex, parietal, aw/cholangioCA, 55F, RP |
| | brain, neocortex, temporal, aw/cholangioCA, 55F, RP |
| | brain, parietal cortex, aw/CHF, 35M |
| | brain, pons, Huntington's, mw/CVA, 57M |
| | brain, striatum/globus pallidus, AD, aw/mets adenoCA, 70F |
| | brain, temporal cortex, aw/CHF, 35M |
| | brain, temporal cortex, aw/aortic aneurysm, 45F |
| | brain, temporal, mw/neuroepithelial tumor, epilepsy, 45M |
| | brain, temporal, polymicrogyria, gliosis, 5M |
| | brain, thalamus, aw/CHF, 35M, NORM |
| | breast tumor, adenoCA, 46F, SUB, m/BRSTNOT33 |
| | breast tumor, lobular CA, 58F, m/BRSTNOT05 |
| | breast, NF breast disease, 35F |
| | breast, mw/ductal adenoCA, intraductal CA, aw/node mets, 57F |
| | cartilage, OA |
| | colon tumor, cecum, carcinoid, 30F |
| | colon, normal/pseudopolyp, Crohn, 16, 26M, pool, lg/N |
| | ear, cochlea, fetal, 16-22w, pool, WM |
| | esophagus tumor, squamous cell CA, 3′, CGAP |
| | eye, retina, 55M, NORM, WM |
| | eye, retina, M/F, TIGR |
| | fat, axillary, aw/breast adenoCA, 73F |
| | gallbladder, cholecystitis, cholelithiasis, 53F |
| | ganglion, dorsal root, thoracic, aw/lymphoma, 32M, NORM |
| | germ cell tumor, pool, NORM, 3′ CGAP |
| | heart, 44M, NORM |
| | heart, coronary artery, CAD, 46M |
| | heart, fetal, 19w, NORM, CGAP/WM/WN |
| | heart, fetal, 8-10w, pool, BI |
| | heart, fetal, M |
| | heart, hypoplastic left, fetal, 23wM |
| | heart, right atrium/muscle wall, Pompe's, 7mM |
| | kidney epithelial transf embryo line, 293-EBNA, lg cDNA |
| | kidney tumor, Wilms', 8mF |
| | kidney tumor, Wilms', pool, WM/WN |
| | kidney, 2dF |
| | kidney, 49M |
| | kidney, aw/anencephaly, fetal, 17wF |
| | kidney, mw/renal cell CA, 43M, m/KIDNTUT14 |
| | kidney, mw/renal cell CA, 65M, m/KIDNTUT15 |
| | kidney, mw/renal cell CA, 8, 53F, pool, NORM |
| | kidney, pool, NORM, 3′ CGAP |
| | kidney, pool, SUB, 3′ CGAP |
| | liver/spleen, fetal, 20wM, NORM, CGAP/WM/WN |
| | lung tumor, myxoid lipoSAR, 65F |
| | lung tumor, neuroendocrine carcinoid, pool, NORM, 3′ CGAP |
| | lung tumor, neuroendocrine carcinoid, pool, SUB, CGAP |
| | lung tumor, squamous cell CA, 64F |
| | lung tumor, squamous cell CA, 65F |
| | lung tumor, squamous cell CA, 69M, m/LUNGNOT15 |
| | lung, 72M |
| | lung, aw/anencephaly, fetal, 20wF |
| | lung, fetal, 19W, NORM, CGAP/WM/WN |
| | lung, fetal, M, RP |
| | lung, mw/adenoCA, 43M |
| | lung, pleura, aw/mets leiomyoSAR to lung, 58F, lg cDNA |
| | lung, pneumonitis, mw/squamous cell CA, 69M, m/LUNGTUT03 |
| | lung, right bronchus, asthmatic, 22-51M/F, pool |
| | microvascular, dermal, endothelial cells, 22F, Untx |
| | microvascular, dermal, endothelial cells, 30F, untreated |
| | mixed tissues, includes tumor, SUB, 3′ CGAP |
| | mixed tissues, includes tumor, pool, SUB, CGAP |
| | multiple sclerosis, 46M, NORM, WM/WN |
| | muscle tumor, striated, alveolar rhabdomyoSAR, 3′/5′ CGAP |
| | muscle, skeletal |
| | neuroganglion tumor, ganglioneuroma, 9M |
| | ovary tumor, papillary serous CA, F, 3′ CGAP |
| | pancreas tumor, adenoCA, 3′ CGAP |
| | pancreas, 2M |
| | paraganglion tumor, paraganglioma, aw/renal cell CA, 46M |
| | parathyroid tumor, adenoma, M/F, NORM, WM |
| | penis, glans, aw/scrotal urothelial CA, M |
| | periph blood, lymphocytes, non-adher PBMC, M/F, 24-hr MLR |
| | pituitary, 16-70M/F, pool |
| | placenta, fetal, 8-9w, pool, NORM, CGAP/WM |
| | placenta, neonatal, F |
| | prostate tumor line, LNCaP, CA, 50M, untreated, 5C-RP |
| | prostate tumor, adenoCA, 57M, m/PROSNOT06 |
| | prostate tumor, adenoCA, 59M, SUB, m/PROSNOT19 |
| | prostate tumor, adenoCA, M, m/PROSNOT28/PROSTMC01/PROSTMT05 |
| | prostate, 28M, NORM |
| | prostate, AH, mw/adenoCA, 48-73M, pool, lg cDNA |
| | prostate, AH, mw/adenoCA, 57M, m/PROSTUT04 |
| | prostate, epithelium, PIN, mw/cancer, M, m/PROETUP02, 3′CGAP |
| | seminal vesicle, aw/prostate adenoCA, 67M |
| | skin, breast, 26F |
| | skin, breast, aw/adenoCA, 70F |
| | skin, epidermal breast keratinocyte line, NHEK, 30F, Untx |
| | sm intestine, 15F |
| | sm intestine, duodenum, aw/pancreatic cystadenoma, 41F |
| | sm intestine, ileum, aw/adenoCA cecum, node mets, 70F |
| | spinal cord, aw/renal failure, 71M, NORM |
| | spleen, ITP, 14M |
| | stomach, fetal, 18wM |
| | teratoCA line, NT2, t/5AZA-3d, SUB |
| | testis, 16M |
| | testis, M, NORM, CGAP/WN |
| | thyroid, lymphocytic thyroiditis, mw/papillary CA, 13F |
| | tonsil, lymphoid hyperplasia, 6M |
| | uterus tumor, leiomyoma, m/UTRSNON03, UTRSNOT12, UTRSTMR01 |
| | uterus, endometrium, mw/cervical dysplasia, 32F, 5RP |
| | uterus, mixed, endometrium/myometrium, 32, 45F, pool, RP |
| | brain stem |
| | kidney |
| #49799: | adrenal glands, aw/anencephaly, fetal, 16wF, lg cDNA |
| | adrenal tumor, pheochromocytoma, 52F, 5RP |
| | aorta, endothelial cells, TIGR |
| | bladder, mw/TC CA, aw/node mets, 58M, m/BLADTUT03 |
| | bone marrow, 16-70M/F, RP |
| | brain tumor, frontal, mets hypernephroma, 58M |
| | brain tumor, frontal, neuronal neoplasm, 32M |
| | brain tumor, glioblastoma, pool, NORM, CGAP |
| | breast tumor, adenoCA, 54F, m/BRSTNOT03 |
| | breast tumor, adenoCA, 55F, m/BRSTNOT02 |
| | breast, NF breast disease, 32F |
| | breast, NF breast disease, 35F |
| | breast, mw/lobular CA, 58F, m/BRSTTUT03 |
| | colon, CUC, 69M |
| | colon, cecum/descending, polyposis, polyp, M/F, pool, SUB |
| | colon, mixed tissues, 16M/13F, pool, NORM |
| | colon, normal/pseudopolyp, Crohn, 16, 26M, pool, lg/N |
| | esophagus tumor, adenoCA, 61M, NORM |
| | fetus, 8-9w, pool, NORM, CGAP/WM/WN |
| | gallbladder, 25F, TIGR |
| | ganglion, dorsal root, cervical, aw/lymphoma, 32M, NORM, EF |
| | ganglion, dorsal root, thoracic, aw/lymphoma, 32M, NORM |
| | germ cell tumor, pool, SUB, 3′ CGAP |
| | heart, coronary artery, CAD, 46M |
| | heart, coronary artery, endothelial cells, 58M |
| | heart, fetal, 19w, NORM, CGAP/WM/WN |
| | heart, right atrium/muscle wall, Pompe's, 7mM, RP |
| | iliac artery, endothelial cells, F, control, untreated |
| | iliac artery, endothelial cells, F, t/1% oxygen 24 hr |
| | iliac artery, endothelial cells, F, untreated |
| | kidney tumor, renal cell CA, 51F |
| | kidney, cortex, mw/renal cell CA, 65M |
| | kidney, right/left, aw/Patau's, fetal, 20wM, pool, lg cDNA |
| | liver tumor, mets neuroendocrine CA, 72M, 5RP, EF |
| | liver, aw/Patau's, anencephaly, fetal, pool, lg cDNA |
| | liver/spleen, fetal, 20wM, NORM, CGAP/WM/WN |
| | liver/spleen, fetal, 20wM, NORM, WM |
| | lung tumor, squamous cell CA, 56M, 5RP |
| | lung, 2M |
| | lung, aw/Patau's, fetal, 20wM |
| | lung, aw/Patau's, fetal, 20wM, lg cDNA |
| | lung, mw/adenoCA, 63M |
| | lung, panacinar emphysema, mw/granuloma, 61M |
| | lymph node tumor, mets melanoma, 3′, CGAP |
| | lymph node, 16mM |
| | microvascular, dermal, endothelial cells, 22F, Untx |
| | microvascular, dermal, endothelial cells, 22F, t/VEGF, EF |
| | microvascular, dermal, endothelial cells, 22F, t/bFGF, EF |
| | microvascular, dermal, endothelial cells, neonatal, M |
| | mixed tissues, fetal lung, testis, B-cell, SUB, 3′ CGAP/WN |
| | mixed tissues, includes mw/tumor, 23w-71M/F, pool, 5RP, EF |
| | mixed tissues, includes tumor, 16w-85M/F, pool, 5RP, EF |
| | mixed tissues, includes tumor, M/F, pool, 5RP, EF |
| | mixed tissues, myometrium, smooth muscle cells, 57M/41F, B |
| | mixed, (melanocytes, uterus, fetal heart), SUB, CGAP/WM/WN |
| | multiple sclerosis, 46M, NORM, WM/WN |
| | muscle, psoas, 12M |
| | ovary, follicular cysts, 28F |
| | penis, corpora cavernosa, M |
| | placenta, aw/hydrocephalus, fetal demise, 16w, 18wM, pool |
| | placenta, aw/hydrocephalus, fetal, 16w, RP |
| | placenta, neonatal, F |
| | placenta, neonatal, F, NORM, WM |
| | prostate, 32M, SUB, 3′ CGAP |
| | prostate, AH, mw/adenoCA, 55M, m/PROSTUT16 |
| | prostate, AH, mw/adenoCA, M, m/PROSTUT13, PROSTUS08/19/20/25 |
| | prostate, BPH, mw/adenoCA, 70M, SUB |
| | sm intestine tumor, ileum, mets ovarian adenoCA, 49F |
| | sm intestine, duodenum tumor line, adenoCA, MCG, EF |
| | sm intestine, jejunum, 16M |
| | spleen, Gaucher's, 22M |
| | stomach, fetal, 20wF |
| | teratoCA line, hNT2, t/RA + MI |
| | testis, 16M |
| | testis, 16M, NORM |
| | testis, M, NORM, CGAP/WN |
| | testis, M, TIGR |
| | thyroid, aw/CHF, 64F |
| | umb cord vein endothelial line, HUV-EC-C, control |
| | umbilical artery, endothelial cells, M, untreated, 5RP, EF |
| | umbilical vein, endothelial cells, HUVEC, t/TNFa-48 hr, 5C-FL, EF |
| | umbilical vein, endothelial cells, pool, WM/WN |
| | umbilical vein, endothelial cells, untreated |
| | uterus, F, NORM, CGAP/WM/WN |
| | uterus, mixed, endometrium/myometrium, 32, 45F, pool, RP |
| | uterus, mw/leiomyomata, 49, 55F, pool, lg cDNA, EF |
| | uterus, myometrium, 43F |
| | endothelial |
| #35689: | Jurkat line, T-cell leukemia, M, Untx, 5C-FL, EF |
| | aorta, 64M, WN |
| | bone marrow tumor line, SH-SY5Y, neuroblastoma, 4F, t/RA |
| | bone marrow tumor line, SH-SY5Y, neuroblastoma, F, untreated |
| | bone tumor, mets prostate cancer, M, 3′ CGAP |
| | brain tumor, frontal, neuronal neoplasm, 32M |
| | brain tumor, mixed types, pool, CGAP |
| | brain, aw/hypoplastic left heart, fetal, 23wM, lg cDNA |
| | brain, aw/muscular atrophy, 3mF, NORM, GEXP |
| | brain, hippocampus, aw/CHF, 35M, NORM |
| | breast tumor line, T-47D, ductal CA, 54F, 5C-FL, EF |
| | cervical tumor line, HeLa S3, adenoCA, 31F, untreated, WM/WN |
| | colon tumor epithelial line, T84, CA, WM |
| | colon tumor line, KM12C, CA, Untx, TIGR |
| | colon tumor, adenoCA, 75M, m/COLNNOT01 |
| | colon tumor, adenoCA, NORM, SUB, CGAP |
| | colon tumor, cecum, adenoCA, 45F |
| | colon tumor, ileocecum, Burkitt lymphoma, 29F, m/COLANOT03 |
| | colon, aw/Patau's, fetal, 20wM, lg cDNA |
| | colon, descending, benign familial polyposis, 16M |
| | colon, normal/pseudopolyp, Crohn, 16, 26M, pool, lg/N |
| | fetus, 8-9W, pool, NORM, CGAP/WM/WN |
| | germ cell tumor, pool, SUB, 3′ CGAP |
| | germ cell tumor, seminoma, teratoma adenoCA, pool, 3′ CGAP |
| | heart coronary artery endothelial cells, 3M, untreated, NORM |
| | heart, fetal, 19W, NORM, CGAP/WM/WN |
| | heart, fetal, 8-10W, pool, BI |
| | kidney tumor, Wilms', 8mF |
| | kidney tumor, Wilms', pool, WM/WN |
| | kidney, 2 dF |
| | kidney, pool, SUB, 3′ CGAP |
| | liver, aw/Pataus's syndrome, fetal, 20wM, 5RP |
| | liver, aw/anencephaly, fetal, 20wF |
| | liver/spleen, fetal, 20wM, NORM, CGAP/WM/WN |
| | lung, idiopathic pulmonary disease, SUB |
| | lymph node, 16mM |
| | mammary, epithelial cells, 21F, untreated |
| | mixed tissues, fetal lung, testis, B-cell, SUB, 3′ CGAP/WN |
| | mixed tissues, myometrium, smooth muscle cells, 57M/41F, B |
| | mixed, (melanocytes, uterus, fetal heart), SUB, CGAP/WM/WN |
| | ovary tumor, mixed types, pooled, F, 3′ CGAP |
| | ovary tumor, papillary serous cystadenoCA 36F, NORM, WM/WN |
| | ovary, aw/cardiomyopathy, 59F |
| | pancreas tumor, adenoCA, 3′ CGAP |
| | periph blood, B-lymphocytes, CLL, pool, NORM, 3′ CGAP |
| | pituitary gland, aw/schizophrenia, COPD, 55M, NORM |
| | prostate tumor, TC CA, 66M, EF |
| | skin, aw/Patau's, fetal, 20wM |
| | skin, dermis, breast, fibroblasts, 31F, t/9CIS RA, lg cDNA, EF |
| | skin, foreskin, melanocytes, M, NORM, WM/WN |
| | sm intestine, fetal, M, RP |
| | spleen, Gaucher's, 22M |
| | spleen, fetal, pool |
| | stomach tumor, adenoCA, poorly differentiated, 3′ CGAP |
| | stomach, 16M |
| | teratoCA line, NT2, t/5AZA-3 d |
| | teratoCA line, NT2, t/cytokines, 5C-FL, EF |
| | teratoCA line, hNT2, t/RA |
| | teratoCA line, hNT2, t/RA + MI, WM/WN |
| | testis tumor, seminoma, 45M |
| | thymus, aw/congenital heart abnormalities, 2F |
| | tonsil, B-lymphocytes, germinal, aw/tonsillitis, NORM, CGAP |
| | umb cord vein endothelial line, HUV-EC-C, control |
| | uterus tumor, serous papillary CA, F, pooled, 3′ CGAP |
| | uterus, F, NORM, CGAP/WM/WN |
| #47225: | adrenal gland, 17M |
| | adrenal gland, 20M |
| | adrenal gland, 20M, NORM |
| | adrenal gland, aw/renal cell CA, 43M |
| | adrenal gland, pool, CHGC, EF |
| | adrenal tumor, adenoma, pool, 3′ CGAP |
| | adrenal tumor, pheochromocytoma, 57F |
| | bladder tumor, TC CA, 72M |
| | brain tumor, oligodendroglioma, NORM, CGAP |
| | brain, 55M, NORM, WM |
| | brain, amygdala, aw/CHF, 35M |
| | brain, aw/hypoplastic heart, fetal, 23wM, pool, RP, EF |
| | brain, aw/hypoplastic left heart, fetal, 23wM |
| | brain, aw/hypoplastic left heart, fetal, 23wM, 5RP |
| | brain, aw/hypoplastic left heart, fetal, 23wM, NORM |
| | brain, caudate nucleus, mw/CVA, 92M |
| | brain, caudate nucleus, schizophrenia, 66F |
| | brain, caudate/putamen/nucleus accumbens, aw/CHF, 35M |
| | brain, cerebellum, aw/COPD, 69M |
| | brain, frontal cortex, aw/CHF, 35M, NORM |
| | brain, frontal, mw/astrocytoma, 40F, m/BRAITUT12/ BRAFTUE03 |
| | brain, hippocampus |
| | brain, hippocampus, AD |
| | brain, hippocampus, aw/CHF, 35M |
| | brain, hypothalamus, Huntington's, mw/CVA, 57M, NORM |
| | brain, mw/oligoastrocytoma, epilepsy, 26M, NORM |
| | brain, occipital, Huntington's, mw/CVA, 57M |
| | brain, pineal gland, aw/AD, 68, 79F, pool, NORM |
| | brain, sensory-motor cortex, aw/CHF, 35M |
| | brain, temporal, gliosis, mw/epilepsy, 27M, lg cDNA |
| | brain, temporal, mw/neuroepithelial tumor, epilepsy, 45M |
| | breast tumor, adenoCA, 55F, m/BRSTNOT02 |
| | breast tumor, ductal, poorly differentiated, F, 3′ CGAP |
| | colon line, pool, LICR, EF |
| | germ cell tumor, pool, SUB, 3′ CGAP |
| | kidney, fetal, 23wM |
| | kidney, mw/renal cell CA, 43M, m/KIDNTUT14 |
| | kidney, pool, NORM, 3′ CGAP |
| | kidney, pool, SUB, 3′ CGAP |
| | kidney, right/left, aw/Patau's, fetal, 20wM, pool, lg cDNA |
| | liver tumor line, C3A, Hepatob, 15M, Untx, NORM |
| | lung tumor, neuroendocrine carcinoid, pool, SUB, CGAP |
| | lung tumor, squamous CA, 50M |
| | lung tumor, squamous cell CA, 68M |
| | mixed tissues, includes tumor, SUB, 3′ CGAP |
| | mixed tissues, includes tumor, Untx cells, M, pool, NORM, EF |
| | mixed, brain/pituitary, Nrml/Huntington's/AD, M/F, pool, 5RP, EF |
| | nervous tissue tumor, CA, pool, LICR, EF |
| | olfactory bulb, aw/CA, 39-85M/F, pool, NORM, EF |
| | ovary tumor, TC CA, 53F |
| | pancreas tumor, adenoCA, 3′ CGAP |
| | pituitary gland, aw/schizophrenia, COPD, 55M, NORM |
| | pituitary, 16-70M/F, pool |
| | prostate, AH, mw/adenoCA, 60M, m/PROSTUT08 |
| | sm intestine tumor, ileum, carcinoid, 42M |
| | teratoCA line, NT2, t/cytokines, 5C-FL, EF |
| | teratoCA line, hNT2, t/mouse leptin, RA |
| | urogenital tumor, TC CA, pool, 3′ CGAP |
| | uterus tumor, endometrial adenoCA, F, pooled, 3′ CGAP |
| #608163: | colon tumor, CA, pool, LICR, EF |
| | sm intestine, fetal, M, 5RP |
| | uterus, endometrium, mw/leiomyoma, 29F, RP |
| | thymus, 3M |
| | CML precursor line, K-562, 53F, Untx, NORM, EF |
| | CML precursor line, K-562, 53F, t/PMA-96 hr |
| | Jurkat line, T-cell leukemia, M, t/PMA-30 min, 5C-FL, EF |
| | Jurkat line, T-cell leukemia, M, t/PMA-30 min, NORM, EF |
| | Jurkat line, T-cell leukemia, untreated, TIGR |
| | adrenal glands, aw/anencephaly, fetal, 16wF, lg cDNA |
| | adrenal tumor, pheochromocytoma, 52F, 5RP |
| | adrenal tumor, pheochromocytoma, 52F, EF |
| | aorta, 64M, WN |
| | aorta, endothelial cells, 33F, treated |
| | blood, dendritic cells, mature, pool, CHGC, EF |
| | bone marrow line, SH-SY5Y, neuroblastoma, 4F, t/6-OHDA, 5RP, EF |
| | bone tumor/line, MG-63, osteoSAR/giant cell, M/F, pool, RP, EF |
| | bone, rib, aw/Patau's, fetal, 20wM |
| | brain, archaeocortex, hippocampus, aw/cholangioCA, 55F, RP |
| | brain, cerebellum, Huntington's, mw/CVA, 57M, RP |
| | brain, cerebellum, TIGR |
| | brain, cerebellum, aw/COPD, left ventricular failure, 70M |
| | brain, choroid plexus, Huntington's, mw/CVA, 57M, RP |
| | brain, frontal cortex, aw/CHF, 35M, NORM |
| | brain, hippocampus, AD, 74M |
| | brain, parietal cortex, aw/CHF, 35M, AMP/N |
| | brain, pons, AD, 74M |
| | brain, substantia nigra, aw/atherosclerosis, CHF, 81F, NORM |
| | brain, temporal, gliosis, mw/epilepsy, 27M, lg cDNA |
| | breast tumor, CA, pool, LICR, EF |
| | breast, papillomatosis, mw/lobular CA, 59F, m/BRSTTUT22, RP |
| | cervical tumor line, HeLa S3, adenoCA, 31F, untreated, WM/WN |
| | colon tumor, juvenile granulosa cell, 3′ CGAP |
| | colon, 16M |
| | colon, ascending, CUC, 25F |
| | colon, pool, LICR, EF |
| | eye, corneal fibroblasts primary line, 76, untreated |
| | fat, abdomen, aw/obesity, 52F |
| | fetus, 8-9w, pool, NORM, CGAP/WM/WN |
| | heart, fetal, 19w, NORM, CGAP/WM/WN |
| | heart, left ventricle, 51F |
| | kidney epithelial transf embryo line, 293-EBNA, t/5AZA |
| | kidney tumor, Wilms', pool, WM/WN |
| | kidney, pool, NORM, 3′ CGAP |
| | kidney, pool, SUB, 3′ CGAP |
| | liver tumor line, C3A, Hepatob, 15M, Untx-24 hr, 5C-FL, EF |
| | liver, primary biliary cirrhosis, 63F, RP |
| | liver/spleen, fetal, 20wM, NORM, WM |
| | lung tumor line, sm/lg cell CA/mucoepidermoid CA, pool, MGC, EF |
| | lung tumor, squamous cell CA, 57M |
| | lung tumor, squamous cell CA, 68M |
| | lung, MGC, EF |
| | mixed tissues, fetal lung, testis, B-cell, SUB, 3′ CGAP/WN |
| | mixed tissues, includes tumor, SUB, 3′ CGAP |
| | ovary tumor, endometrioid CA, 62F |
| | ovary tumor, seroanaplastic CA, 52F |
| | ovary tumor, serous papillary adenoCA, F, 3′ CGAP |
| | pancreas tumor, adenoCA, 3′ CGAP |
| | pancreas, islet cells, pool |
| | pancreatic tumor line, adenoCA, untreated, WM/WN |
| | periph blood, promonocyte line, THP-1, AML, t/5AZA, SUB |
| | placenta, pool, LICR, EF |
| | prostate tumor, cancer, 45M, m/PROETMP01/02, CGAP |
| | prostate, MGC, EF |
| | skin tumor line, melanoma/squamous cell CA, pool, MGC, EF |
| | skin, foreskin, melanocytes, M, NORM, WM/WN |
| | sm intestine, ileum, Crohn's, 18F |
| | spleen, 8M |
| | stomach tumor, CA, pool, LICR, EF |
| | stomach tumor, adenoCA, poorly differentiated, 3′ CGAP |
| | stomach, aw/esophagus adenoCA, 23w-61M, pool, AMP |
| | stomach, fetal, 20wF |
| | teratoCA line, NT2, t/5AZA-3d, SUB |
| | testis, 16M |
| | testis, M, NORM, CGAP/WN |
| | thymus tumor, malignant thymoma, 56F |
| | thymus, aw/congenital heart abnormalities, 2F, RP |
| | thyroid tumor, papillary CA, 3′ CGAP |
| | thyroid, aw/CHF, 64F |
| | tonsil, lymphoid hyperplasia, 6M |
| | uterus tumor, endometrial adenoCA, F, pooled, 3′ CGAP |
| | uterus, endometrium, type I defect, 30, 32, 36F, pool |
| | uterus, myometrium, mw/leiomyoma, 41F, RP, m/UTRSTUT05 |
| | uterus, tumor line, adenoCA/leiomyoSAR, pool, MGC, EF |
| | clavicle, osteoblasts, 40M, untreated, lg cDNA, EF |
| | Skin, melanotic melanoma. |
| #40497: | brain, neocortex, parietal, aw/cholangioCA, 55F, RP |
| | breast, PF changes, 40F |
| | colon tumor, CA, pool, LICR, EF |
| | colon tumor, rectum, adenoCA, mw/tubular adenoma, 50M, 5RP |
| | colon, mucosa, Crohn, pool, NORM, CGAP |
| | colon, normal/pseudopolyp, Crohn, 16, 26M, pool, lg/N |
| | colon, transverse, benign familial polyposis, 16M |
| | esophagus tumor, adenoCA, 61M, NORM |
| | liver tumor, hepatoma, 50M |
| | liver, MGC, EF |
| | liver, aw/astrocytoma, 32F |
| | mixed tissue tumor, head/neck, CA, pool, LICR, EF |
| | seminal vesicle, aw/prostate adenoCA, 63M, 5RP |
| | sm intestine tumor, ileum, mets ovarian adenoCA, 49F |
| | sm intestine, 31F, RP |
| | sm intestine, aw/Patau's syndrome, fetal, 20wM, 5RP |
| | sm intestine, duodenum, 16M |
| | sm intestine, ileum, Crohn's, 18F |
| | sm intestine, ileum, chronic inflammation, 29F, 5RP, EF |
| | sm intestine, ileum, mw/CUC, 42M |
| | sm intestine, ileum, mw/carcinoid, adenoCA, F, pool, lg cDNA |
| | liver |
| | small intestine |
| #46144: | CML precursor line, K-562, 53F, Untx, 5C-FL, EF |
| | adrenal gland, pool, CHGC, EF |
| | bone marrow tumor line, CML/AML, pool, MGC, EF |
| | bone marrow tumor line, SH-SY5Y, neuroblastoma, 4F, t/6-OHDA |
| | brain tumor, benign meningioma, 35F, 5RP |
| | brain tumor, mixed, pool, MGC, EF |
| | brain, archaeocortex, hippocampus, aw/cholangioCA, 55F, RP |
| | brain, globus pallidus/substantia innominata, aw/CHF, 35M |
| | brain, thalamus, aw/CHF, 35M |
| | breast tumor line, T-47D, ductal CA, 54F, 5C-FL, EF |
| | breast, PF breast disease, 57F |
| | breast, PF changes, mw/adenoCA, 55F, m/BRSTTUT01 |
| | breast, PF changes, mw/adenoCA, intraductal CA, 43F |
| | colon tumor, adenoCA, 60M, m/COLNNOT07/08/09/11 |
| | femoral artery, aw/chondroSAR, 68M |
| | kidney epithelial transf embryo line, 293-EBNA, serum starv |
| | kidney epithelial transf embryo line, 293-EBNA, tf/E2F1, DP1 |
| | liver tumor, hepatocellular CA, pool, CHGC, EF |
| | liver/spleen, fetal, 20wM, NORM, CGAP/WM/WN |
| | lung, mw/mets thyroid CA, 79M, m/LUNGTUT02 |
| | microvascular, dermal, endothelial cells, 30F, untreated |
| | mixed tissues, fetal lung, testis, B-cell, SUB, 3′ CGAP/WN |
| | multiple sclerosis, 46M, NORM, WM/WN |
| | neuroganglion tumor, ganglioneuroma, 9M |
| | placenta, aw/hydrocephalus, fetal demise, 16w, 18wM, pool |
| | placenta, fetal, 8-9w, pool, NORM, CGAP/WM |
| | prostate, AH, mw/adenoCA, node mets, 55M, lg/N, m/PROSTUT16 |
| | prostate, MGC, EF |
| | prostate, mw/adenoCA, 68M, m/PROSTUT18 |
| | sm intestine, ileum, aw/adenoCA cecum, node mets, 70F, RP |
| | spinal cord, cervical, aw/lymphoma, 32M |
| | teratoCA line, hNT2, t/RA + MI |
| | tonsil, B-lymphocytes, germinal, aw/tonsillitis, NORM, CGAP |
| | uterus tumor, leiomyoma, m/UTRSNON03, UTRSNOT12, UTRSTMR01 |
| | uterus tumor, serous papillary CA, F, pooled, 3′ CGAP |
| | uterus, F, NORM, CGAP/WM/WN |
| | uterus, endometrium, mw/cervical dysplasia, 32F, 5RP |
| | uterus, endometrium, type I defect, 28F |
| | Jurkat line, Frac Nuc, T-cell leukemia, M, t/antiCD3, NORM, EF |
| | adrenal gland, 20M, NORM |
| | brain, hypothalamus, Huntington's, mw/CVA, 57M, NORM |
| | breast tumor, lobular CA, 59F, m/BRSTNOR01, BRSTNOT16 |
| | colon tumor line, KM12C, CA, Untx, TIGR |
| | colon tumor, adenoCA, 3′, CGAP |
| | colon, cecum/descending, polyposis, polyp, M/F, pool, SUB |
| | fetus, 8-9w, pool, NORM, CGAP/WM/WN |
| | gallbladder, cholecystitis, cholelithiasis, 21M |
| | germ cell tumor, pool, NORM, 3′ CGAP |
| | germ cell tumor, pool, SUB, 3′ CGAP |
| | germ cell tumor, seminoma, teratoma adenoCA, pool, 3′ CGAP |
| | heart, left atrium, 51F |
| | kidney tumor, Wilms', 8mF |
| | kidney tumor, clear cell type cancer, pool, SUB, CGAP |
| | kidney tumor, renal cell CA, 46M, 5RP |
| | kidney, 2dF |
| | kidney, pool, SUB, 3′ CGAP |
| | lung tumor, adenoCA, 70F |
| | lung tumor, neuroendocrine carcinoid, pool, NORM, 3′ CGAP |
| | lung tumor, neuroendocrine carcinoid, pool, SUB, CGAP |
| | mixed tissues, includes tumor, SUB, 3′ CGAP |
| | mixed tumor, Wilm's/brain mets, pool, 3′ CGAP |
| | penis, corpus cavernosum, M |
| | prostate tumor, adenoCA, 61M |
| | sm intestine tumor, ileum, carcinoid, 42M |
| | spleen, aw/pancreas neuroendocrine CA, 65F |
| | testis, 16M |
| | testis, M, NORM, CGAP/WN |
| | testis, M, pool, LICR, EF |
| | uterus tumor, endometrial adenoCA, F, pooled, 3′ CGAP |
| | liver |
| | Uterus |
| | Brain, glioblastoma |
| | prostate |
| #38609: | brain, aw/hypoplastic left heart, fetal, 23wM, 5C-RP |
| | brain, aw/hypoplastic left heart, fetal, 23wM, RP |
| | brain, hippocampus, AD, 77F, EF |
| | liver tumor, mets neuroendocrine CA, 62F, m/ LIVRTMR01 |
| | ovary tumor line, adenoCA, pool, MGC, EF |
| | placenta tumor line, chorioCA, fetal, pool, MGC, EF |
| | placenta, aw/hydrocephalus, fetal, 16w, RP |
| | teratoCA line, hNT2, untreated |
| | testis, M, pool, LICR, EF |
| | CML precursor line, K-562, 53F, Untx, NORM, EF |
| | Raji line, Burkitt, 11M, t/PMA, Iono-30 min, 5C-FL, EF |
| | bladder tumor, TC CA, 60M, m/BLADNOT05 |
| | bladder tumor, microscopic foci TC CA, 58M |
| | bladder, 11M |
| | bladder, 11M, RP |
| | brain, acute/chronic multiple sclerosis, pool |
| | brain, cingulate, aw/MI, 85F, AMP/N |
| | brain, frontal, Huntington's, mw/CVA, 57M |
| | brain, infant, 10wF, NORM, WM |
| | brain, neocortex, frontal, aw/cholangioCA, 55F, RP |
| | breast tumor, lobular CA, 58F, m/BRSTNOT05 |
| | breast, mw/neoplasm, 36F |
| | colon line, pool, LTCR, EF |
| | colon tumor, adenoCA, pool, NORM, 3′ CGAP |
| | colon, epithelium, 13F, RP |
| | fat, mesentery, aw/diverticulosis, diverticulitis, 71M |
| | fat, mixed tissues, aw/breast adenoCA, 38-73M/F, pool, NORM |
| | ganglion, dorsal root, cervical, aw/lymphoma, 32M |
| | ganglion, dorsal root, cervical, aw/lymphoma, 32M, NORM, EF |
| | ganglion, dorsal root, thoracic, aw/lymphoma, 32M, NORM |
| | germ cell tumor, pool, SUB, 3′ CGAP |
| | heart, fetal, 18wM |
| | heart, fetal, 19w, NORM, CGAP/WM/WN |
| | heart, fetal, 8-10w, pool, BI |
| | kidney epithelial transf embryo line, 293-EBNA, t/5AZA |
| | kidney tumor, renal cell CA, pool, 3′/5′ CGAP |
| | kidney, cortex, mw/renal cell CA, 65M |
| | liver tumor line, C3A, Hepatob, 15M, Untx, NORM |
| | lung tumor line, sm/lg cell CA/mucoepidermoid CA, pool, MGC, EF |
| | lung, aw/Patau's, fetal, 20wM |
| | lung, aw/Patau's, fetal, 20wM, lg cDNA |
| | lung, mw/adenoCA, 63M |
| | lymph node tumor, mets melanoma, 3′, CGAP |
| | mixed tissues, includes tumor, pool, SUB, CGAP |
| | ovary, endometriosis, aw/leiomyomata, 39F, NORM, EF |
| | pancreas tumor line, adenoCA/epitheloid CA, pool, MGC, EF |
| | periph blood, eosinophils, asthma, M/F |
| | periph blood, promonocyte line, THP-1, AML, t/5AZA |
| | pituitary gland, aw/schizophrenia, COPD, 55M, NORM |
| | placenta, aw/hydrocephalus, fetal demise, 16w, 18wM, pool |
| | placenta, fetal, 21wF |
| | placenta, fetal, 8-9w, pool, NORM, CGAP/WM |
| | placenta, fetal, M, WM |
| | placenta, neonatal, F, NORM, WM |
| | placenta, pool, LICR, EF |
| | prostate, 32M, SUB, 3′ CGAP |
| | seminal vesicle, aw/prostate adenoCA, 67M |
| | sm intestine, aw/Patau's syndrome, fetal, 20wM, 5RP |
| | sm intestine, ileum, 4F |
| | sm intestine, mw/carcinoid, aw/node mets, 59M, RP |
| | stomach, 55M |
| | synovium, rheuA, 75M/56F, pool, NORM |
| | testis, M, NORM, CGAP/WN |
| | umb cord blood, mononuclear cells, M/F, t/G-CSF, lg cDNA |
| | umb cord vein endothelial line, HUV-EC-C, t/cytokine, LPS |
| | uterus tumor, serous papillary CA, F, pooled, 3′ CGAP |
| | uterus, aw/ovarian follicular cysts, 34F |
| | uterus, endometrium, mw/endometrial polyp, 35F |
| | uterus, myometrium, mw/leiomyoma, 41F, NORM, m/UTRSTUT05 |
| | spleen |
| #34119: | muscle tumor line, rhabdomyosarcoma, MGC, EF |
| | brain, hippocampus, AD, 74M |
| | brain, mw/oligoastrocytoma, epilepsy, 26M, SUB |
| | lung, fetal, M, RP |
| | lung, mw/spindle cell carcinoid, 62F |
| | ovary tumor, TC CA, 53F |
| | uterus, endometrium, mw/leiomyoma, 29F, NORM |
| | Jurkat line, T-cell leukemia, M, t/PMA-30 min, NORM, EF |
| | Raji line, Burkitt, 11M, t/PMA, Iono-30 min, 5C-FL, EF |
| | UCB, derived dendritic cells, pool, untreated/treated, NORM |
| | adrenal gland, 20M |
| | adrenal gland, aw/pituitary neoplasm, 61F |
| | adrenal gland, mixed, Nrml/pheochromocytoma, pool, lg cDNA, EF |
| | adrenal tumor, adenoma, pool, 3′ CGAP |
| | bladder tumor, TC CA, 58M, m/BLADNOT09 |
| | bone marrow tumor line, SH-SY5Y, pool, Untx/t/6OHDA, NORM, EF |
| | brain tumor, frontal, astrocytoma, 40F, m/BRAINOT14 |
| | brain tumor, oligodendroglioma, CGAP |
| | brain, amygdala/entorhinal cortex, aw/CA, 55F, RP, EF |
| | brain, aw/hypoplastic left heart, fetal, 23wM, NORM |
| | brain, choroid plexus, Huntington's, mw/CVA, 57M, RP |
| | brain, frontal cortex, schizophrenic, 34M, RP, WM/WN |
| | brain, frontal, Huntington's, mw/CVA, 57M |
| | brain, hippocampus, mw/intracranial hemorrhage, 72F |
| | brain, medulla, Huntington's, mw/CVA, 57M |
| | brain, neurogenic tumor line, SK-N-MC, neuroepithelioma, 14F |
| | brain, occipital, Huntington's, mw/CVA, 57M |
| | brain, parietal cortex, aw/CHF, 35M |
| | brain, temporal, mw/neuroepithelial tumor, epilepsy, 45M |
| | breast tumor line, T-47D, ductal CA, 54F, 5C-FL, EF |
| | breast tumor line, T47D, ductal CA, 54F |
| | breast tumor, ductal CA, 43F, m/BRSTTMT01 |
| | breast, 56F |
| | breast, NF breast disease, 32F |
| | breast, NF changes, mw/ductal adenoCA, 40-57F, pool, lg cDNA |
| | colon tumor, CA, pool, LICR, EF |
| | colon tumor, adenoCA, 3′ CGAP |
| | colon tumor, adenoCA, NORM, SUB, CGAP |
| | colon, CUC, 69M |
| | colon, mw/adenoCA, aw/node mets, 60M, m/COLNTUT16 |
| | colon, normal/pseudopolyp, Crohn, 16, 26M, pool, lg/N |
| | coronary artery, smooth muscle cells, 3M |
| | epiglottis, aw/papillary thyroid CA, 71M |
| | esophagus, mw/adenoCA, aw/node mets, 53M |
| | gallbladder, cholecystitis, cholelithiasis, 18F |
| | ganglion, dorsal root, cervical, aw/lymphoma, 32M |
| | ganglion, dorsal root, cervical, aw/lymphoma, 32M, NORM, EF |
| | germ cell tumor, pool, SUB, 3′ CGAP |
| | germ cell tumor, seminoma, teratoma adenoCA, pool, 3′ CGAP |
| | heart, 44M |
| | heart, 65M |
| | heart, aorta, 39M, lg cDNA, EF |
| | heart, aw/Patau's syndrome, fetal, 20wM, FL-EN, EF |
| | heart, coronary artery, CAD, 46M |
| | heart, fetal, 19w, NORM, CGAP/WM/WN |
| | heart, left ventricle, mw/myocardial infarction, 56M |
| | kidney epithelial transf embryo line, 293-EBNA, Untx, 5C-FL, EF |
| | kidney epithelial transf embryo line, 293-EBNA, Untx, NORM, EF |
| | kidney, fetal, 19-23w, M/F, lg cDNA |
| | kidney, medulla/cortex, mw/renal cell CA, 53F, m/KIDNTUT16 |
| | kidney, mw/benign cyst, nephrolithiasis, 42F |
| | kidney, pool, NORM, 3′ CGAP |
| | kidney, pool, SUB, 3′ CGAP |
| | liver, 49M |
| | liver, CD34+ progenitor cells, fetal, 20wM |
| | liver/spleen, fetal, 20wM, NORM, CGAP/WM/WN |
| | lung tumor line, NCI-H69, sm cell CA, 55M, untreated, WM/WN |
| | lung tumor, neuroendocrine carcinoid, pool, NORM, 3′ CGAP |
| | lung tumor, squamous cell CA, 69M, m/LUNGNOT15 |
| | lung, 12M |
| | lung, 2M |
| | lung, 35F, 5RP |
| | lung, asthma, 15M |
| | lymph node tumor, follicular lymphoma, 3′ CGAP |
| | lymph node, 11F |
| | mast cells, liver, fetal, 22w |
| | microvascular, dermal, endothelial cells, 22F, t/VEGF, EF |
| | mixed tissues, fetal lung, testis, B-cell, SUB, 3′ CGAP/WN |
| | mixed, (melanocytes, uterus, fetal heart), SUB, CGAP/WM/WN |
| | multiple sclerosis, 46M, NORM, WM/WN |
| | nose, nasal polyps, lg cDNA, EF |
| | ovary tumor, mucinous cystadenoCA, 43F, m/OVARNOT03 |
| | ovary tumor, serous papillary, F, CGAP |
| | ovary, aw/leiomyomata, 52F |
| | pancreas tumor, adenoCA, 3′ CGAP |
| | pancreas, 17F, NORM |
| | pancreas, 5M |
| | pancreatic tumor, anaplastic CA, 45F |
| | paraganglion tumor, paraganglioma, aw/renal cell CA, 46M |
| | parathyroid tumor, adenoma, M/F, NORM, WM |
| | periph blood, dendritic cells, t/TNF |
| | periph blood, promonocyte line, THP-1, AML, untreated |
| | placenta tumor line, chorioCA, fetal, pool, MGC, EF |
| | placenta, aw/hydrocephalus, fetal, 16w, FL-EN, EF |
| | placenta, pool, LICR, EF |
| | probable astrocytes, M/F, untreated |
| | prostate tumor, adenoCA, 57M, m/PROSNOT06 |
| | prostate tumor, adenoCA, 59M, SUB, m/PROSNOT19 |
| | prostate tumor, adenoCA, M, m/PROSNOT28/PROSTMC01/PROSTMT05 |
| | prostate, 32M, SUB, 3′ CGAP |
| | prostate, AH, mw/adenoCA, 57M, m/PROSTUT04 |
| | prostate, AH, mw/adenoCA, 66M, m/PROSTUT10 |
| | prostate, epithelial cells, 17M, untreated, NORM |
| | skin tumor line, melanoma/squamous cell CA, pool, MGC, EF |
| | skin, dermis, breast, fibroblasts, 31F, untreated |
| | sm intestine, 8M |
| | sm intestine, fetal, M, RP |
| | sm intestine, ileum, 8F |
| | sm intestine, ileum, Crohn's, 18F |
| | spinal cord, cervical, aw/lymphoma, 32M |
| | stomach tumor, adenoCA, poorly differentiated, 3′ CGAP |
| | stomach, pool, LICR, EF |
| | submandibular gland, aw/sialoadenitis, 49F, lg cDNA, EF |
| | teratoCA line, NT2, t/cytokines, 5C-FL, EF |
| | teratoCA line, hNT2, untreated |
| | testis tumor, embryonal CA, 31M, 5RP |
| | testis, 16M |
| | testis, M, NORM, CGAP/WN |
| | thymus, aw/parathyroid adenoma, 21M |
| | thymus, hyperplasia, aw/myasthenia gravis, 16F |
| | tonsil, lymphoid hyperplasia, 6M/9F, pool, lg cDNA, EF |
| | umb cord blood, mononuclear cells, M/F, t/G-CSF, lg cDNA |
| | umb cord vein endothelial line, HUV-EC-C, control |
| | uterus tumor, endometrial adenoCA, F, pooled, 3′ CGAP |
| | uterus tumor, endometrial, F, TIGR |
| | uterus tumor, leiomyomata/adenosquamousCA, F, pool, lg cDNA, EF |
| | uterus tumor, serous papillary CA, F, pooled, 3′ CGAP |
| | uterus, F, NORM, CGAP/WM/WN |
| | uterus, cervix, 40F |
| | uterus, cervix, cervicitis, mw/leiomyoma, 29F, 5RP |
| | uterus, endometrium, type I defect, 30, 32, 36F, pool |
| | kidney |
| | bladder tumor, TC CA, 66M, m/BLADNOT06 |
| | heart, fetal, 8-10w, pool, BI |
| | lung, idiopathic pulmonary disease, SUB |
| | mixed tissues, includes tumor, 16w-85M/F, pool, 5RP, EF |
| | mixed tissues, includes tumor, M/F, pool, 5RP, EF |
| | prostate, AH, mw/adenoCA, 55M, m/PROSTUT16, lg cDNA |
| | teratoCA line, hNT2, t/mouse leptin, 9cis RA-6d, lg cDNA |
| | ovary, dermoid cyst/aw/leiomyoma, 22, 47F, pool, lg cDNA |
| | Muscle, rhabdomyosarcoma |
| | brain |
| | teratocarcinoma (NT2) |
| #33890: | CML precursor line, K-562, 53F, Untx, 5C-FL, EF |
| | T-lymphocytes, CD4+, pool, t/anti-CD3 antibodies |
| | chest wall, soft tissue, mw/adenoCA, aw/COPD, 63M |
| | colon, mw/adenoCA, aw/node mets, 60M, m/COLNTUT16 |
| | lung tumor, adenoCA, 47M |
| | lung, idiopathic pulmonary disease, SUB |
| | lymph node, B-lymphocytes, germinal center, pool, MGC, EF |
| | mixed tissues, fetal lung, testis, B-cell, SUB, 3′ CGAP/WN |
| | multiple sclerosis, 46M, NORM, WM/WN |
| | ovary tumor, mucinous cystadenoma, 51F |
| | ovary tumor, papillary serous cystadenoCA 36F, NORM, WM/WN |
| | periph blood, eosinophils, t/IL-5 |
| | placenta, aw/hydrocephalus, fetal, 16w, RP |
| | placenta, fetal, 21wF |
| | sm intestine, fetal, M, RP, EF |
| | synovium, wrist, rheuA, 62F |
| | testis tumor line, embryonal CA, pool, MGC, EF |
| | testis, M, NORM, CGAP/WN |
| | thymus, hyperplasia, aw/myasthenia gravis, 16F |
| | thyroid, AH, mw/papillary CA, 22F, 5RP, EF |
| | tonsil, B-lymphocytes, germinal, aw/tonsillitis, NORM, CGAP |
| | tonsil, lymphoid hyperplasia, 6M/9F, pool, lg cDNA, EF |
| | umb cord blood, T-lymphocytes, Th2 cells, untreated |
| | uterus, endometrium, aw/cystocele, 38F |
| | whole blood, myeloid cells, CML, pool, 3′ CGAP |
| | (U937) |
| #51500: | brain tumor line, DU 145, mets prostate CA, 69M, Untx, 5C-FL, EF |
| | lung, aw/Patau's, fetal, 20wM, lg cDNA |
| | placenta, aw/hydrocephalus, fetal demise, 16w, 18wM, pool |
| | thyroid, mw/adenomatous goiter with follicular adenoma, 18F |
| | Jurkat line, T-cell leukemia, M, t/PMA, Iono-1 hr, FL-EN, EF |
| | Jurkat line, T-cell leukemia, untreated, TIGR |
| | adrenal tumor, pheochromocytoma, 52F, EF |
| | bladder tumor line, CA/TC CA/papilloma, pool, MGC, EF |
| | bone marrow tumor line, CML/AML, pool, MGC, EF |
| | bone marrow tumor line, SH-SY5Y, neuroblastoma, F, untreated |
| | bone marrow tumor, CA, pool, LICR, EF |
| | bone tumor line, osteoSAR, MGC, EF |
| | brain tumor, benign meningioma, 35F, 5RP |
| | brain tumor, frontal, astrocytoma, 17F |
| | brain tumor, frontal, meningioma, 68M |
| | brain, aw/hypoplastic left heart, fetal, 23wM, 5RP |
| | brain, aw/hypoplastic left heart, fetal, 23wM, NORM |
| | brain, aw/hypoplastic left heart, fetal, 23wM, lg cDNA |
| | brain, frontal cortex, aw/lung CA, 77M |
| | brain, frontal, fetal, 5mM, 3′/5′, WN |
| | brain, mixed tissues, gliosis, 27, 35M, pool, lg cDNA |
| | breast tumor line, T-47D, ductal CA, 54F, 5C-FL, EF |
| | breast tumor, adenoCA, 46F, m/BRSTNOT17 |
| | breast, 35F |
| | breast, NF changes, mw/ductal adenoCA, 40-57F, pool, lg cDNA |
| | colon tumor, CA, pool, LICR, EF |
| | colon tumor, adenoCA, 60M, m/COLNNOT07/08/09/11 |
| | fetus, 8-9w, pool, NORM, CGAP/WM/WN |
| | fetus, 9w, TIGR |
| | ganglion, dorsal root, cervical, aw/lymphoma, 32M, NORM, EF |
| | heart, fetal, 19w, NORM, CGAP/WM/WN |
| | kidney epithelial transf embryo line, 293-EBNA, tf/E2F1, DP1 |
| | kidney tumor, renal cell CA, 46M, 5RP |
| | kidney, fetal, 23wM |
| | lung tumor line, sm/lg cell CA/mucoepidermoid CA, pool, MGC, EF |
| | lung, 72M |
| | lung, mw/adenoCA, 43M |
| | lymphocytes, activated Th2 cells, 6-hr AB |
| | mixed tissue tumor, head/neck, CA, pool, LICR, EF |
| | muscle, forearm, mw/intramuscular hemangioma 38F |
| | muscle, skeletal, mw/malignant hyperthermia, WM/WN |
| | osteogenic tumor line, Saos-2, SAR, 11F, untreated |
| | ovary tumor line, adenoCA, pool, MGC, EF |
| | ovary tumor, seroanaplastic CA, 52F |
| | ovary, endometriosis, 24F |
| | periph blood, granulocytes, M/F, t/GM-CSF |
| | periph blood, granulocytes, M/F, t/fMLP |
| | periph blood, lymphocytes, non-adher PBMC, M/F, t/LPS |
| | periph blood, macrophages, adher PBMC, M/F, MLR-24 hr |
| | periph blood, promonocyte line, THP-1, AML, untreated |
| | prostate, AH, mw/adenoCA, 50M, m/PROSTUT01 |
| | prostate, AH, mw/adenoCA, 55M, m/PROSTUT16 |
| | prostate, AH, mw/adenoCA, node mets, 55M, lg/N, m/PROSTUT16 |
| | prostate, BPH, mw/adenoCA, PIN, 59M |
| | skin tumor line, melanoma/squamous cell CA, pool, MGC, EF |
| | sm intestine, fetal, M, RP |
| | spinal cord, aw/renal failure, 71M, NORM |
| | stomach, fetal, 18wM |
| | stomach, mw/adenoCA, node mets, 52M, m/STOMTUT01 |
| | stomach, pool, LICR, EF |
| | teratoCA line, NT2, t/cytokines, NORM, EF |
| | teratoCA line, hNT2, untreated |
| | thymus, hyperplasia, aw/myasthenia gravis, 16F |
| | umb cord blood, T-lymphocytes, Th2 cells, untreated |
| | umb cord blood, mononuclear cells, t/IL-5 |
| | umbilical vein, endothelial cells, HUVEC, t/TNFa-48 hr, 5C-FL, EF |
| | uterus tumor, leiomyoma, 41F |
| | uterus, F, NORM, CGAP/WM/WN |
| | uterus, cervix tumor line, CA, pool, MGC, EF |
| | uterus, endometrium, mw/cervical dysplasia, 32F, FL-EN, EF |
| | uterus, endometrium, mw/cervicitis, leiomyoma, pool, lg cDNA |
| | uterus, myometrium, mw/leiomyoma, 41F, RP, m/UTRSTUT05 |
| | heart, fetal, 8-10w, pool, BI |
| | kidney tumor, Wilms', pool, WM/WN |
| | placenta, choriocarcinoma |
| | CML precursor line, K-562, 53F, Untx |
| | CML precursor line, K-562, 53F, Untx, NORM, EF |
| | CML precursor line, K-562, 53F, t/5AZA-72 hr |
| | CML precursor line, K-562, 53F, t/9cis RA-13d |
| | CML precursor line, K-562, 53F, t/PMA-96 hr |
| | Jurkat line, Frac Nuc, T-cell leukemia, M, t/antiCD3, NORM, EF |
| | Jurkat line, T-cell leukemia, M, t/PMA |
| | PBMC, 60M, untreated |
| | T-B lymphoblast line, leukemia, untreated |
| | T-lymphocyte tumor, lymphoma, TIGR |
| | T-lymphocytes, allogenic anergic, 40-50M, t/OKT3 3 day |
| | T-lymphocytes, allogenic, 40-50M, untreated |
| | UCB, CD34+ hematopoietic stem cells, CHGC, EF |
| | UCB, derived dendritic cells, M |
| | UCB, derived dendritic cells, M, t/PMA, Iono-5 hr |
| | UCB, derived dendritic cells, pool, untreated/treated, NORM |
| | adipose, pericecal, aw/cecal tubular adenoma, 55F |
| | adrenal gland, 20M, FL-EN, EF |
| | adrenal gland, mixed, Nrml/pheochromocytoma, pool, lg cDNA, EF |
| | adrenal tumor, adrenal cortical CA, 49M, 5RP, EF |
| | adrenal tumor, mets renal cell CA, 50M |
| | adrenal tumor, pheochromocytoma, 43F, m/ADRENOT11 |
| | adrenal tumor, pheochromocytoma, 57F |
| | aorta, 64M, WN |
| | aorta, adventitia, 48M |
| | aorta, adventitia, 65F |
| | astrocytes, M/F, t/cytokines 12 hr |
| | astrocytes, M/F, t/cytokines 4-6 hr |
| | bladder tumor, TC CA, 66M, m/BLADNOT06 |
| | bladder tumor, TC CA, 80F, m/BLADNOT03 |
| | bladder tumor, microscopic foci TC CA, 58M |
| | bladder, 78F |
| | bladder, mw/TC CA, aw/node mets, 58M, m/BLADTUT03 |
| | blood, dendritic cells, mature, pool, CHGC, EF |
| | bone marrow tumor line, SH-SY5Y, neuroblastoma, 4F, t/RA |
| | bone marrow, 16-70M/F |
| | bone marrow, CD34+ hematopoietic stem cells, pool, CHGC, EF |
| | bone marrow, CD34+ progenitor cells, M, AMP |
| | bone tumor, Ewing's SAR, CGAP |
| | bone tumor, rib, mets osteoSAR, 16M |
| | bone, rib, aw/Patau's, fetal, 20wM, lg cDNA |
| | brain stem, aw/DMt1, 72M |
| | brain stem, aw/DMt1, 72M, NORM |
| | brain tumor, anaplastic oligodendroglioma, pool, NORM, CGAP |
| | brain tumor, frontal, astrocytoma, 40F, m/BRAINOT14 |
| | brain tumor, frontal, astrocytoma, 47M |
| | brain tumor, meningioma, pool, 3′, CGAP |
| | brain tumor, mixed types, pool, CGAP |
| | brain tumor, mixed, pool, MGC, EF |
| | brain tumor, parietal, glioma, 43F |
| | brain, 55M, NORM, WM |
| | brain, acute/chronic multiple sclerosis, pool |
| | brain, allocortex, cingulate, aw/cholangioCA, 55F, RP |
| | brain, amygdala/entorhinal cortex, aw/CA, 55F, RP, EF |
| | brain, archaeocortex, hippocampus, aw/cholangioCA, 55F, RP |
| | brain, astrocytes, fetal, 22wF, untreated |
| | brain, aw/hypoplastic left heart, fetal, 23wM |
| | brain, aw/hypoplastic left heart, fetal, 23wM, 5RP, EF |
| | brain, aw/hypoplastic left heart, fetal, 23wM, FL-EN, EF |
| | brain, cerebellum, AD, 74M |
| | brain, cerebellum, aw/COPD, 69M |
| | brain, choroid plexus, Huntington's, mw/CVA, 57M, RP |
| | brain, cingulate, aw/MI, 85F, AMP/N |
| | brain, corpus callosum, AD, 74M |
| | brain, dentate nucleus, aw/CHF, 35M |
| | brain, dentate nucleus, aw/cholangioCA, 55F, RP |
| | brain, fetal, 23wM |
| | brain, fetal, 23wM, FL-EN, EF |
| | brain, fetal, 24wF, TIGR |
| | brain, frontal cortex, aw/CHF, 35M, NORM |
| | brain, frontal cortex, schizophrenic, 34M, RP, WM/WN |
| | brain, frontal, Huntington's, mw/CVA, 57M |
| | brain, frontal, polymicrogyria, gliosis, 5M |
| | brain, globus pallidus/substantia innominata, aw/CHF, 35M |
| | brain, hippocampus, AD |
| | brain, infant, 10wF, NORM, WM |
| | brain, infant, F, TIGR |
| | brain, medulla, aw/CHF, 35M, AMP |
| | brain, mixed tissues, aw/CHF, 35M, pool, lg/N |
| | brain, mixed tissues, aw/aortic aneurysm, 45F |
| | brain, mw/oligoastrocytoma, epilepsy, 26M |
| | brain, mw/oligoastrocytoma, epilepsy, 26M, NORM |
| | brain, neurogenic tumor line, SK-N-MC, neuroepithelioma, 14F |
| | brain, parietal cortex, aw/CHF, 35M |
| | brain, pineal gland, aw/AD, COPD, 79F |
| | brain, sensory-motor cortex, aw/CHF, 35M |
| | brain, substantia nigra, aw/atherosclerosis, CHF, 81F, NORM |
| | brain, temporal cortex, aw/CHF, 35M |
| | brain, temporal cortex, aw/aortic aneurysm, 45F |
| | brain, temporal, gliosis, mw/epilepsy, 27M, lg cDNA |
| | brain, temporal, polymicrogyria, gliosis, 5M |
| | brain, thalamus, aw/CHF, 35M, NORM |
| | breast tumor line, T47D, ductal CA, 54F |
| | breast tumor line, adenoCA, MGC, EF |
| | breast tumor, CA, pool, LICR, EF |
| | breast tumor, adenoCA, 46F, SUB, m/BRSTNOT33 |
| | breast tumor, adenoCA, 55F, m/BRSTNOT02 |
| | breast tumor, ductal CA, 43F, m/BRSTTMT01 |
| | breast tumor, ductal, F, pool, NORM, 3′/5′ CGAP |
| | breast tumor, lobular CA, 58F, m/BRSTNOT05 |
| | breast, 56F |
| | breast, NF breast disease, 46F |
| | breast, PF changes, 40F |
| | breast, PF changes, mw/adenoCA, 45F, m/BRSTTUT08 |
| | breast, PF changes, mw/adenoCA, 55F, m/BRSTTUT01 |
| | breast, PF changes, mw/adenoCA, intraductal CA, 43F |
| | breast, PF changes, mw/ductal adenoCA, 54F, m/BRSTTUT02 |
| | breast, mw/ductal CA, CA in situ, aw/node mets, 62F |
| | breast, mw/lobular CA, 58F, m/BRSTTUT03 |
| | breast, mw/neoplasm, 36F |
| | breast, papillomatosis, mw/lobular CA, 59F, m/BRSTTUT22 |
| | breast, pool, LICR, EF |
| | bronchial epithelium line, NHBE, 54M, Untx |
| | bronchial epithelium line, NHBE, 54M, Untx, NORM |
| | bronchus, 15M |
| | bronchus, smooth muscle cells, 21M, untreated |
| | cartilage, OA |
| | cervical tumor line, HeLa, adenoCA, 31F, t/Na butyrate 24 hr |
| | cervix, cervicitis, 35F |
| | chest wall, soft tissue, mw/adenoCA, aw/COPD, 63M |
| | clavicle, osteoblasts, 40M, untreated |
| | colon polyp, aw/adenoCA, tubulovillous adenoma, 40F |
| | colon tumor epithelial line, T84, CA, WM |
| | colon tumor line, adenoCA, pool, MGC, EF |
| | colon tumor, adenoCA, 3′ CGAP |
| | colon tumor, adenoCA, 75M, m/COLNNOT01 |
| | colon tumor, adenoCA, pool, NORM, 3′ CGAP |
| | colon tumor, adenoCA, pool, NORM, 3′/5′ CGAP |
| | colon tumor, cecum, adenoCA, 45F |
| | colon tumor, cecum, adenoCA, 70F |
| | colon tumor, cecum, carcinoid, 30F |
| | colon tumor, ileocecum, Burkitt lymphoma, 29F, m/COLANOT03 |
| | colon tumor, sigmoid, adenoCA, 62M, m/COLNNOT16 |
| | colon, 16M |
| | colon, CUC, 69M |
| | colon, appendix, aw/leiomyomata, 37F |
| | colon, ascending, CUC, 74M, EF |
| | colon, ascending, mw/CUC, 28M |
| | colon, aw/Patau's, fetal, 20wM, lg cDNA |
| | colon, aw/anencephaly, fetal, 20wF |
| | colon, cecum polyp, aw/adenoCA, 67F |
| | colon, mw/adenoCA, aw/node mets, 60M, NORM, m/COLNTUT16 |
| | colon, normal/pseudopolyp, Crohn, 16, 26M, pool, lg/N |
| | colon, pool, LICR, EF |
| | colon, transverse, Crohn's, 26M |
| | colon, transverse, benign familial polyposis, 16M |
| | colon, ulcerative colitis, 16M |
| | coronary artery, smooth muscle cells, 3M, t/TNF, IL-1, SUB |
| | ear, cochlea, fetal, 16-22w, pool, WM |
| | embryo, 8w, TIGR |
| | eye tumor line, retinoblastoma, pool, MGC, EF |
| | eye, retina, M/F, TIGR |
| | fat, breast, aw/fibrosis, 38F |
| | fat, mesentery, aw/diverticulosis, diverticulitis, 71M |
| | fibroblasts, aortic adventitia, 65F, untreated |
| | gallbladder tumor, squamous cell CA, 78F |
| | gallbladder, cholecystitis, cholelithiasis, 21M |
| | ganglion, dorsal root, cervical, aw/lymphoma, 32M |
| | ganglion, dorsal root, thoracic, aw/lymphoma, 32M |
| | ganglion, dorsal root, thoracic/lumbar, aw/lymphoma, 32M |
| | germ cell tumor, pool, NORM, 3′ CGAP |
| | germ cell tumor, yolk sac, 3′ CGAP |
| | heart tumor, left atrium, myxoma, 43M |
| | heart, 65M |
| | heart, coronary artery, CAD, 46M |
| | heart, coronary artery, endothelial cells, 58M |
| | heart, coronary artery, plaque, pool |
| | heart, fetal, 18wM |
| | heart, hypoplastic left, fetal, 23wM |
| | heart, left ventricle, 31M |
| | heart, left ventricle, mw/myocardial infarction, 56M |
| | heart, right atrium, 39M |
| | iliac artery, endothelial cells, F, t/1% oxygen 24 hr |
| | iliac artery, endothelial cells, F, untreated |
| | kidney epithelial transf embryo line, 293-EBNA, t/5AZA, SUB |
| | kidney tumor line, CA/adenoCA/hypernephroma, pool, MGC, EF |
| | kidney tumor, Wilms', 8mF |
| | kidney tumor, clear cell type cancer, pool, NORM, 3′ CGAP |
| | kidney tumor, renal cell CA, 51F |
| | kidney tumor, renal cell CA, 65M m/KIDNNOT19 |
| | kidney tumor, renal cell CA, pool, 3′/5′ CGAP |
| | kidney, 2dF |
| | kidney, 64F |
| | kidney, aw/anencephaly, fetal, 17wF |
| | kidney, aw/hypoplastic left heart, fetal, 23wM |
| | kidney, cortex, mw/renal cell CA, 65M |
| | kidney, fetal, TIGR |
| | kidney, interstitial nephritis, aw/bladder TC CA, 63M, 5RP |
| | kidney, mw/benign cyst, nephrolithiasis, 42F |
| | kidney, mw/renal cell CA, 65M, m/KIDNTUT15 |
| | liver tumor line, C3A, Hepatob, 15M, Untx, NORM |
| | liver tumor line, adenoCA, MGC, EF |
| | liver tumor, mets colon adenoCA, 51F |
| | liver, CD34+ progenitor cells, fetal, 20wM |
| | liver, aw/Patau's, anencephaly, fetal, pool, lg cDNA |
| | liver, pool, CHGC, EF |
| | liver, primary biliary cirrhosis, 63F |
| | liver, primary biliary cirrhosis, 63F, RP |
| | liver/spleen, fetal, 20wM, NORM, CGAP/WM/WN |
| | liver/spleen, fetal, 20wM, NORM, WM |
| | lung tumor line, NCI-H69, sm cell CA, 55M, untreated, WM/WN |
| | lung tumor, adenoCA, 47M |
| | lung tumor, adenoCA, 70F |
| | lung tumor, mets granulosa cell tumor, 80F |
| | lung tumor, myxoid lipoSAR, 65F |
| | lung tumor, neuroendocrine carcinoid, pool, NORM, 3′ CGAP |
| | lung tumor, neuroendocrine carcinoid, pool, SUB, CGAP |
| | lung tumor, squamous cell CA, 50M |
| | lung tumor, squamous cell CA, pooled, NORM, CGAP |
| | lung, 15F |
| | lung, 17F |
| | lung, 47M |
| | lung, 72M, WM/WN |
| | lung, MGC, EF |
| | lung, aw/anencephaly, fetal, 17wF |
| | lung, aw/anencephaly, fetal, 20wF |
| | lung, fetal, 19w, NORM, CGAP/WM/WN |
| | lung, fetal, 23wM |
| | lung, idiopathic pulmonary disease, NORM |
| | lung, mw/adenoCA, 53M, m/LUNGTUT17 |
| | lung, mw/adenoCA, aw/node, diaphragm mets, 63F |
| | lung, mw/mets osteoSAR, aw/pleura mets, 58M |
| | lung, mw/mets osteoSAR, aw/pleura mets, 58M, NORM |
| | lung, mw/mets thyroid CA, 79M, m/LUNGTUT02 |
| | lung, mw/spindle cell carcinoid, 62F |
| | lung, pleura, aw/mets leiomyoSAR to lung, 58F, lg cDNA |
| | lymph node tumor line, lymphoma/Burkitt, pool, MGC, EF |
| | lymph node tumor, follicular lymphoma, 3′ CGAP |
| | lymph node tumor, gastric, mets esophageal adenoCA, 61M |
| | lymph node, necrotic, aw/lung squamous cell CA, 67M |
| | lymph node, peripancreatic, aw/pancreatic adenoCA, 65M |
| | lymph nodes, 42F |
| | lymphocytes, PBMC, M, 96-hr MLR |
| | lymphocytes, activated Th1 cells, 6-hr AB |
| | lymphocytes, nonactivated Th1 cells |
| | mammary, epithelial cells, 21F, untreated |
| | mesentery tumor, sigmoid, mets mixed-mullerian tumor, 61F |
| | microvascular, dermal, endothelial cells, 18F, untreated |
| | microvascular, dermal, endothelial cells, neonatal, M |
| | mixed tissue, head/neck, pool, LICR, EF |
| | mixed tissues, fetal lung, testis, B-cell, SUB, 3′ CGAP/WN |
| | mixed tissues, includes tumor, SUB, 3′ CGAP |
| | mixed tissues, myometrium, smooth muscle cells, 57M/41F, B |
| | mixed tumor, Wilm's/brain mets, pool, 3′ CGAP |
| | mixed, (melanocytes, uterus, fetal heart), SUB, CGAP/WM/WN |
| | mixed, brain/pituitary, Nrml/Huntington's/AD, M/F, pool, 5RP, EF |
| | multiple sclerosis, 46M, NORM, WM/WN |
| | muscle tumor line, rhabdomyosarcoma, MGC, EF |
| | muscle, calf, mw/gangrene, aw/atherosclerosis, 67M |
| | muscle, skeletal, leg, 19F, GEXP |
| | muscle, thigh, mw/lipoSAR, 58M, RP |
| | nervous tissue tumor, CA, pool, LICR, EF |
| | nervous tissue, pool, LICR, EF |
| | neuroganglion tumor, ganglioneuroma, 9M |
| | nose, nasal polyps, aw/asthma, 78M, pool, NORM |
| | ovary tumor, CA, F, pool, LICR, EF |
| | ovary tumor, TC CA, 53F |
| | ovary tumor, adenoCA, 58F |
| | ovary tumor, endometrioid CA, 62F |
| | ovary tumor, mets colon adenoCA, 58F, NORM |
| | ovary tumor, mixed types, pooled, F, 3′ CGAP |
| | ovary tumor, mucinous cystadenoCA, 43F, m/OVARNOT03 |
| | ovary tumor, papillary serous cystadenoCA 36F, NORM, WM/WN |
| | ovary tumor, serous papillary adenoCA, F, 3′ CGAP |
| | ovary, aw/cardiomyopathy, 59F |
| | ovary, aw/cardiomyopathy, 59F, NORM |
| | ovary, aw/leiomyoma, 47F |
| | ovary, aw/leiomyomata, 36F |
| | ovary, aw/leiomyomata, 47F |
| | ovary, aw/leiomyomata, 52F |
| | ovary, endometriosis, aw/leiomyomata, 39F |
| | ovary, follicular cysts, 28F |
| | ovary, mw/follicular cysts, 28F |
| | ovary, mw/mucinous cystadenoCA, 43F, m/OVARTUT01 |
| | pancreas tumor, adenoCA, 3′ CGAP |
| | pancreas, aw/Patau's, fetal, 20wM |
| | pancreas, fetal, 23wM |
| | pancreas, islet cells, pool |
| | pancreatic tumor, anaplastic CA, 45F |
| | paraganglion tumor, paraganglioma, aw/renal cell CA, 46M |
| | parathyroid tumor, adenoma, M/F, NORM, WM |
| | penis tumor, squamous cell CA, 64M, 5RP |
| | penis, corpora cavernosa, M |
| | penis, corpora cavernosa, aw/scrotal urothelial CA, M |
| | penis, corpus cavernosum, M |
| | penis, glans, aw/scrotal urothelial CA, M |
| | periph blood, B-lymphocytes, CLL, pool, NORM, 3′ CGAP |
| | periph blood, T-lymphocytes, CD8+, 63M, untreated |
| | periph blood, eosinophils, t/IL-5 |
| | periph blood, lymphocytes, non-adher PBMC, 24M |
| | periph blood, macrophages, adher PBMC, M/F, t/LPS |
| | periph blood, monocytes, 42F, t/IL-10, LPS |
| | periph blood, promonocyte line, THP-1, AML, 1M, t/tuberculosis |
| | periph blood, promonocyte line, THP-1, AML, control |
| | peritoneum tumor, neuroendocrine CA, 66F |
| | pituitary gland, pool, CHGC, EF |
| | pituitary, 16-70M/F, pool |
| | placenta tumor line, chorioCA, fetal, pool, MGC, EF |
| | placenta, MGC, EF |
| | placenta, aw/hydrocephalus, fetal, 16w |
| | placenta, aw/hydrocephalus, fetal, 16w, RP |
| | placenta, aw/hydrocephalus, fetal, 16w/18wM, pool, RP |
| | placenta, fetal, 21wF |
| | placenta, fetal, 8-9w, pool, NORM, CGAP/WM |
| | placenta, neonatal, F, NORM, WM |
| | probable astrocytes, M/F, untreated |
| | probable periph blood, eosinophils, asthma, M/F |
| | prostate stroma, fibroblasts, fetal, 26wM, untreated |
| | prostate tumor line, CA/adenoCA, pool, MGC, EF |
| | prostate tumor line, LNCaP, CA, 50M, untreated |
| | prostate tumor, TC CA, 66M, EF |
| | prostate tumor, adenoCA, 50M, m/PROSNOT02 |
| | prostate tumor, adenoCA, 57M, m/PROSNOT06 |
| | prostate tumor, adenoCA, 58M |
| | prostate tumor, adenoCA, 59M, SUB, m/PROSNOT19 |
| | prostate tumor, adenoCA, 59M, m/PROSNOT19 |
| | prostate tumor, adenoCA, 66M, m/PROSNOT15, PROSDIN01 |
| | prostate tumor, adenoCA, 68M, m/PROSTMT03 |
| | prostate tumor, adenoCA, 69M, m/PROSNOT07 |
| | prostate tumor, cancer, 45M, m/PROETMP01/02, CGAP |
| | prostate, 21M, TIGR |
| | prostate, 28M, NORM |
| | prostate, 32M, 3′ CGAP |
| | prostate, 32M, NORM, 3′/5′ CGAP |
| | prostate, 32M, SUB, 3′ CGAP |
| | prostate, AH, aw/bladder TC CA, 58M |
| | prostate, AH, mw/adenoCA, 53M |
| | prostate, AH, mw/adenoCA, 57M, m/PROSTUT04 |
| | prostate, AH, mw/adenoCA, 60M, m/PROSTUT08 |
| | prostate, AH, mw/adenoCA, 65M |
| | prostate, AH, mw/adenoCA, 66M, m/PROSTUT17 |
| | prostate, AH, mw/adenoCA, 67M |
| | prostate, AH, mw/adenoCA, M, m/PROSTUT13, PROSTUS08/19/20/25 |
| | prostate, BPH, mw/adenoCA, 70M, SUB |
| | prostate, epithelial cells, 17M, untreated |
| | prostate, epithelium, PIN, mw/cancer, 45M, m/PROETUP02, CGAP |
| | prostate, mw/adenoCA, 68M, m/PROSTUT18 |
| | pulmonary artery, endothelial cells, 10M, t/TNF, IL-1 |
| | seminal vesicle, aw/adenoCA, 58M |
| | skin, aw/Patau's, fetal, 20wM |
| | skin, breast and fetal, aw/intraductal CA, pool |
| | skin, breast, 26F |
| | skin, epidermal breast keratinocyte line, NHEK, 30F, Untx |
| | skin, foreskin, melanocytes, M, NORM, WM/WN |
| | skin, leg, keratinocytes, neonatal, M |
| | skull tumor, chondroid chordoma, 30F |
| | sm intestine tumor, ileum, mets endometrial adenoCA, 64F |
| | sm intestine, 15F |
| | sm intestine, 55F |
| | sm intestine, 8M |
| | sm intestine, duodenum tumor line, adenoCA, MCG, EF |
| | sm intestine, duodenum, 8F |
| | sm intestine, fetal, 8-16M/F, pool, NORM |
| | sm intestine, ileum, 4F |
| | sm intestine, ileum, 8F |
| | sm intestine, ileum, Crohn's, 18F |
| | sm intestine, ileum, aw/adenoCA cecum, node mets, 70F, RP |
| | sm intestine, ileum, mw/CUC, 25F |
| | sm intestine, jejunum, 8F |
| | soft tissue tumor, thigh, mets myxoid lipoSAR, 34F |
| | spleen tumor, Hodgkin's, 45M |
| | spleen, 29M |
| | spleen, 2M |
| | spleen, Gaucher's, 22M |
| | spleen, ITP, 8, 14M, pool, lg cDNA |
| | spleen, aw/pancreas neuroendocrine CA, 65F |
| | stomach tumor, adenoCA, poorly differentiated, 3′ CGAP |
| | synovium, elbow, rheuA, 51F |
| | synovium, knee, OA, 82F |
| | teratoCA line, NT2, t/5AZA-3d |
| | teratoCA line, NT2, t/5AZA-3d, SUB |
| | teratoCA line, hNT2, t/RA + MI, WM |
| | teratoCA line, hNT2, t/RA + MI, WM/WN |
| | teratoCA line, hNT2, t/RA, WM/WN |
| | teratoCA line, hNT2, t/mouse leptin, 9cis RA-6d, lg cDNA |
| | teratoCA line, hNT2, untreated, WM/WN |
| | testis tumor line, embryonal CA, pool, MGC, EF |
| | testis tumor, germ cell, seminoma, M, 3′/5′ CGAP |
| | testis, 16M |
| | testis, M, NORM, CGAP/WN |
| | testis, M, pool, LICR, EF |
| | testis, aw/cirrhosis, 37M |
| | thymus, 3M |
| | thymus, aw/anencephaly, fetal, 17wF |
| | thymus, aw/congenital heart abnormalities, 2F |
| | thymus, fetal, pool, NORM, CGAP |
| | thyroid tumor, follicular adenoma, 17M |
| | thyroid, lymphocytic thyroiditis, mw/papillary CA, 30F |
| | tongue tumor, CA, m/TONGTMP01, CGAP |
| | tonsil, B-lymphocytes, germinal, aw/tonsillitis, NORM, CGAP |
| | tonsil, lymphoid hyperplasia, 6M |
| | umb cord vein endothelial line, HUV-EC-C, shear stress |
| | umbilical artery, endothelial cells, neonatal, M, untreated |
| | umbilical vein, endothelial cells, pool, WM/WN |
| | unspecified tumor, CA, aw/Denys-drash, pool, LICR, EF |
| | ureter tumor, TC CA, 64M, 5RP |
| | uterus tumor, CGAP |
| | uterus tumor, endometrial adenoCA, F, pooled, 3′ CGAP |
| | uterus tumor, endometrial, F, TIGR |
| | uterus tumor, leiomyoma, 34F |
| | uterus tumor, serous papillary CA, F, pooled, 3′ CGAP |
| | uterus, F, TIGR |
| | uterus, Nrml/tumor, F, pool, LICR, EF |
| | uterus, aw/liver & breast cancer, 46F |
| | uterus, aw/ovarian follicular cysts, 34F |
| | uterus, endometrium, F, pool |
| | uterus, endometrium, aw/adenoCA, 50F |
| | uterus, endometrium, mw/endometrial polyp, 35F |
| | uterus, endometrium, mw/leiomyoma aw/endometriosis, 48F |
| | uterus, endometrium, mw/leiomyoma, 29F, NORM |
| | uterus, endometrium, type I defect, 30, 32, 36F, pool |
| | uterus, mixed, endometrium/myometrium, 32, 45F, pool, RP |
| | uterus, myometrium, mw/adenoCA, 50F |
| | uterus, myometrium, mw/leiomyoma, 41F, NORM, m/UTRSTUT05 |
| | uterus, myometrium, mw/leiomyoma, 43F |
| | uterus, tumor line, adenoCA/leiomyoSAR, pool, MGC, EF |
| | white blood cells, 45F |
| | whole blood, myeloid cells, CML, pool, 3′ CGAP |
| | breast tumor, lobular CA, 59F, m/BRSTNOR01, BRSTNOT16 |
| | breast, mw/ductal adenoCA, intraductal CA, aw/node mets, 57F |
| | colon, cecum, benign familial polyposis, 16M |
| | fetal brain |
| | Lymph, Burkitt lymphoma |
| #699545: | Jurkat line, T-cell leukemia, untreated, TIGR |
| | T-lymphocytes, allogenic anergic, 40-50M, t/OKT3 3 day |
| | adenoid, inflamed, 3 |
| | adipose tumor, lipoSAR, 3′ CGAP |
| | adrenal tumor, pheochromocytoma, 52F, EF |
| | bladder tumor, microscopic foci TC CA, 58M |
| | bladder, chronic cystitis, aw/urethral adenoCA, 73M |
| | bone marrow tumor line, SH-SY5Y, neuroblastoma, 4F, t/6-OHDA |
| | brain tumor line, DU 145, mets prostate CA, 69M, Untx, 5C-FL, EF |
| | brain tumor, frontal, meningioma, 68M |
| | brain, 55M, NORM, WM |
| | brain, allocortex/neocortex, cingulate, aw/CA, 55F, RP |
| | brain, archaeocortex, hippocampus, aw/cholangioCA, 55F, RP |
| | brain, aw/hypoplastic heart, fetal, 23wM, pool, RP, EF |
| | brain, aw/hypoplastic left heart, fetal, 23wM |
| | brain, aw/hypoplastic left heart, fetal, 23wM, 5RP |
| | brain, aw/hypoplastic left heart, fetal, 23wM, lg cDNA |
| | brain, aw/spinal muscular atrophy, 72dF, 3′ SIK/TIGR |
| | brain, cerebellum, AD, 74M |
| | brain, cerebellum, Huntington's, mw/CVA, 57M, RP |
| | brain, cerebellum, aw/COPD, 69M |
| | brain, cingulate, aw/MI, 85F, AMP/N |
| | brain, fetal, 23wM |
| | brain, frontal cortex, schizophrenic, 34M, RP, WM/WN |
| | brain, infant, 10wF, NORM, WM |
| | brain, mixed, archaecortex/hippocampus, 55F, pool, AMP/N |
| | brain, mw/oligoastrocytoma, epilepsy, 26M, NORM |
| | brain, temporal, gliosis, mw/epilepsy, cerebral palsy, 27M |
| | breast tumor, adenoCA, 54F, m/BRSTNOT03 |
| | breast, PF changes, mw/adenoCA, intraductal CA, 43F |
| | breast, mw/ductal adenoCA, intraductal CA, aw/node mets, 57F |
| | breast, mw/neoplasm, 36F |
| | clavicle, osteoblasts, 40M, untreated |
| | colon tumor line, KM12C, CA, Untx, TIGR |
| | colon tumor, adenoCA, carcinoid, M/F, pool, NORM |
| | colon tumor, juvenile granulosa cell, 3′ CGAP |
| | colon tumor, rectum, adenoCA, mw/tubular adenoma, 50M, 5RP |
| | colon, aw/Patau's, fetal, 20wM, lg cDNA |
| | colon, cecum, Crohn's, 31M |
| | colon, descending, benign familial polyposis, 16M |
| | fetus, 9w, TIGR |
| | fibroblast line, GD23A, t/radiation 30 min |
| | ganglion, dorsal root, thoracic/lumbar, aw/lymphoma, 32M |
| | heart, aorta, 39M, 5RP |
| | heart, aorta, 39M, lg cDNA, EF |
| | heart, aw/Patau's syndrome, fetal, 20wM, 5RP |
| | heart, fetal, M |
| | heart, right atrium/muscle wall, Pompe's, 7mM |
| | kidney epithelial transf embryo line, 293-EBNA, t/5AZA, SUB |
| | kidney tumor, renal cell CA, 51F |
| | kidney tumor, renal cell CA, pool, 3′/5′ CGAP |
| | kidney, 2dF |
| | kidney, cortex, mw/renal cell CA, 65M, 5RP |
| | kidney, fetal, 19-23w, M/F, lg cDNA |
| | kidney, interstitial nephritis, aw/bladder TC CA, 63M, 5RP |
| | liver tumor line, C3A, Hepatob, 15M, t/PB-48 hr |
| | liver tumor, mets neuroendocrine CA, 62F, m/ LIVRTMR01 |
| | liver/spleen, fetal, 20wM, NORM, WM |
| | lung tumor, mets granulosa cell tumor, 80F |
| | lung, aw/Patau's, fetal, 20wM |
| | lung, idiopathic pulmonary disease, SUB |
| | lung, mw/mets osteoSAR, aw/pleura mets, 58M |
| | lung, mw/mets osteoSAR, aw/pleura mets, 58M, NORM |
| | lung, pleura, aw/mets leiomyoSAR to lung, 58F, lg cDNA |
| | lung, right bronchus, asthmatic, 22-51M/F, pool |
| | megakaryoblast line, MEG-01, CML, 55M, untreated |
| | mixed tissues, fetal lung, testis, B-cell, SUB, 3′ CGAP/WN |
| | mixed tissues, includes tumor, M/F, pool, NORM, EF |
| | mixed tissues, includes tumor, SUB, 3′ CGAP |
| | mixed tissues, includes tumor, pool, SUB, CGAP |
| | mixed tissues, includes tumor, treated cells, pool, NORM, EF |
| | mixed tissues, myometrium, smooth muscle cells, 57M/41F, B |
| | mixed tumor, Wilm's/brain mets, pool, 3′ CGAP |
| | mixed, (melanocytes, uterus, fetal heart), SUB, CGAP/WM/WN |
| | multiple sclerosis, 46M, NORM, WM/WN |
| | muscle, arm, ALS, 74F |
| | muscle, skeletal, aw/Krabbe, 11mF |
| | muscle, skeletal, mw/malignant hyperthermia, WM/WN |
| | nose, nasal polyp, eosinophilia, aw/asthma, 78M |
| | olfactory bulb, aw/CA, 39-85M/F, pool, PlyAT, EF |
| | osteogenic tumor line, Saos-2, SAR, 11F, untreated |
| | ovary tumor, mucinous cystadenoCA, 43F, m/OVARNOT03 |
| | ovary tumor, papillary serous CA, 64F, WM/WN |
| | ovary, aw/leiomyoma, 47F |
| | ovary, endometriosis, 37F, EF |
| | ovary, endometriosis, aw/multiple leiomyomata, 47F, 5C-RP |
| | pancreas, 8M |
| | pancreas, islet cells, pool |
| | penis, aw/Patau's, fetal, 20wM, lg cDNA, EF, EF |
| | penis, corpus cavernosum, M |
| | periph blood, granulocytes, M/F, t/fMLP |
| | periph blood, macrophages, adher PBMC, M/F |
| | peritoneum tumor, neuroendocrine CA, 66F |
| | pituitary gland, aw/schizophrenia, COPD, 55M, NORM |
| | pituitary, 16-70M/F, pool |
| | pituitary, aw/colon cancer, 46M |
| | placenta, aw/hydrocephalus, fetal, 16w, 5RP |
| | placenta, aw/hydrocephalus, fetal, 16w, FL-EN, EF |
| | placenta, aw/hydrocephalus, fetal, 16w, RP |
| | placenta, fetal, 8-9w, pool, NORM, CGAP/WM |
| | prostate stroma, fibroblasts, fetal, 26wM, untreated, NORM |
| | prostate tumor, adenoCA, 59M, SUB, m/PROSNOT19 |
| | prostate, 28M, NORM |
| | prostate, 32M, NORM, 3′/5′ CGAP |
| | prostate, 32M, SUB, 3′ CGAP |
| | prostate, AR, mw/adenoCA, 48-73M, pool, lg cDNA |
| | prostate, epithelial cells, 17M, untreated, AMP |
| | seminal vesicle, aw/prostate adenoCA, 67M |
| | skin, aw/Patau's, fetal, 20wM |
| | skin, dermis, breast, fibroblasts, 31F, t/9CIS RA-20 hr |
| | skin, dermis, breast, fibroblasts, 31F, untreated |
| | sm intestine, duodenum, aw/pancreatic cystadenoma, 41F |
| | sm intestine, fetal, 23wM, FL-EN, EF |
| | sm intestine, fetal, M, RP |
| | sm intestine, ileum, aw/adenoCA cecum, node mets, 70F, RP |
| | spinal cord, base medulla, Huntington's, aw/CVA, 57M |
| | spleen, aw/hypoplastic left heart, fetal, 23wM, FL-EN, EF |
| | spleen, fetal, 23wM |
| | stomach tumor, adenoCA, poorly differentiated, 3′ CGAP |
| | stomach, 16M |
| | stomach, fetal, 20wF |
| | teratoCA line, hNT2, t/RA + MI, WM |
| | testis, 10-61M, pool, lg cDNA |
| | testis, 16M |
| | testis, M, NORM, CGAP/WN |
| | testis, aw/cirrhosis, 37M |
| | thymus, 3M |
| | thymus, aw/congenital heart abnormalities, 2F, RP |
| | thyroid, mw/follicular adenoma, 28F |
| | tonsil, B-lymphocytes, germinal, aw/tonsillitis, NORM, CGAP |
| | uterus tumor, endometrial adenoCA, F, pooled, 3′ CGAP |
| | uterus tumor, leiomyoma, m/UTRSNON03, UTRSNOT12, UTRSTMR01 |
| | uterus tumor, serous papillary CA, F, pooled, 3′ CGAP |
| | uterus, F, NORM, CGAP/WM/WN |
| | uterus, endometrium, 32F |
| | uterus, endometrium, F, pool |
| | uterus, endometrium, mw/cervical dysplasia, 32F, 5RP |
| | uterus, endometrium, mw/cervicitis, leiomyoma, pool, lg cDNA |
| | uterus, endometrium, mw/endometrial polyp, 35F |
| | uterus, endometrium, mw/leiomyoma, 29F, RP |
| | uterus, endometrium, type I defect, 30, 32, 36F, pool |
| | uterus, endometrium, type II defect, endometriosis, F |
| | uterus, mixed, endometrium/myometrium, 32, 45F, pool, RP |
| | uterus, myometrium, 43F |
| | uterus, myometrium, mw/leiomyoma, 41F, RP, m/UTRSTUT05 |
| | uterus, myometrium, mw/leiomyoma, 43F |
| | adrenal gland, aw/pituitary neoplasm, 61F |
| | brain, hypothalamus, Huntington's, mw/CVA, 57M, NORM |
| | colon tumor, adenoCA, 75M, m/COLNNOT01 |
| | ear, cochlea, fetal, 16-22w, pool, WM |
| | kidney epithelial transf embryo line, 293-EBNA, tf/bgal |
| | liver tumor line, C3A, Hepatob, 15M, t/MCA, SUB |
| | lung tumor, squamous cell CA, 69M, m/LUNGNOT15 |
| | lung, asthma, 17M |
| | penis, corpora cavernosa, M |
| | periph blood, B-lymphocytes, CLL, pool, NORM, 3′ CGAP |
| | seminal vesicle, aw/prostate adenoCA, 63M, 5RP |
| | testis |
| #623354: | Raji line, Burkitt, 11M, t/PMA, Iono-30 min, 5C-FL, EF |
| | kidney epithelial transf embryo line, 293-EBNA, Untx, 5C-FL, EF |
| | testis; ovary |
| #728098: | mixed tissues, includes tumor, treated cells, pool, NORM, EF |
| | muscle, skeletal, MGC, EF |
| | Skeletal Muscle |
| #619844: | adrenal tumor, cortical CA, aw/hilar mets, 52M, RP |
| | brain, aw/hypoplastic heart, fetal, 23wM, pool, RP, EF |
| | brain, temporal cortex, aw/aortic aneurysm, 45F, RP |
| | breast tumor line, Hs 578T, ductal CA, 74F, t/EGF-8 hr, 5C-FL, EF |
| | eye tumor line, retinoblastoma, pool, MGC, EF |
| | lung tumor, squamous cell CA, pooled, NORM, CGAP |
| | placenta, pool, LICR, EF |
| | uterus tumor, CA, pool, LICR, EF |
| | uterus, endometrium, mw/cervical dysplasia, 32F, 5RP |
| | uterus, mw/leiomyomata, 49, 55F, pool, lg cDNA, EF |
| | uterus, myometrium, mw/adenoCA, 59F, RP |
| #729509: | brain, aw/hypoplastic left heart, fetal, 23wM |
| | brain, aw/hypoplastic left heart, fetal, 23wM, NORM |
| | brain, cerebellum, Huntington's, mw/CVA, 57M, RP |
| | brain, mw/oligoastrocytoma, epilepsy, 26M, NORM |
| | breast tumor, adenoCA, 46F, m/BRSTNOT17 |
| | breast, PF changes, mw/ductal adenoCA, 54F, m/BRSTTUT02 |
| | colon, mw/adenoCA, aw/COPD, 75M, m/COLNTUT02 |
| | heart, aorta, 12F |
| | heart, coronary artery, CAD, 46M |
| | kidney, MGC, EF |
| | kidney, pool, SUB, 3′ CGAP |
| | lung, mw/endobronchial carcinoid, 33M |
| | lung, mw/mets osteoSAR, aw/pleura mets, 58M, NORM |
| | mammary, epithelial cells, 21F, untreated, NORM |
| | mixed tissues, fetal lung, testis, B-cell, SUB, 3′ CGAP/WN |
| | mixed tissues, includes tumor, SUB, 3′ CGAP |
| | muscle, glossal, mw/squamous cell CA, 41F |
| | ovary, aw/cardiomyopathy, 59F |
| | penis, corpora cavernosa, M |
| | prostate stroma, fibroblasts, fetal, 26wM, untreated |
| | prostate, AH, mw/adenoCA, 67M |
| | skin, aw/Patau's, fetal, 20wM |
| | sm intestine, fetal, M, 5RP |
| | sm intestine, mw/carcinoid, aw/node mets, 59M, RP |
| | soft tissue tumor, spinal schwannoma, 35M |
| | testis, 26M |
| | testis, M, NORM, CGAP/WN |
| | tonsil, B-lymphocytes, germinal, aw/tonsillitis, NORM, CGAP |
| | uterus tumor, endometrial adenoCA, F, pooled, 3′ CGAP |
| | uterus, myometrium, mw/multiple leiomyomata, 45F |
| #35582: | UCB, derived dendritic cells, pool, untreated/treated, NORM |
| | bone marrow tumor, CA, pool, LICR, EF |
| | colon tumor, adenoCA, pool, NORM, 3′ CGAP |
| | gallbladder, cholecystitis, cholelithiasis, 18F |
| | heart tumor, left atrium, myxoma, 43M |
| | kidney epithelial transf embryo line, 293-EBNA, t/5AZA, SUB |
| | lung, 72M |
| | lung, fetal, 23wM |
| | lung, mw/endobronchial carcinoid, 33M |
| | mixed tissue tumor, head/neck, CA, pool, LICR, EF |
| | periph blood, granulocytes, M/F, t/GM-CSF |
| | periph blood, granulocytes, M/F, t/LPS |
| | periph blood, granulocytes, M/F, t/fMLP |
| | thymus, fetal, pool, NORM, CGAP |
| | umb cord blood, mononuclear cells |
| | neutrophil (normal human peripheral blood-derived) |
| #742686: | brain, archaeocortex, hippocampus, aw/cholangioCA, 55F, RP |
| | brain, aw/hypoplastic left heart, fetal, 23wM, 5RP |
| | brain, dentate nucleus, aw/CHF, 35M, lg cDNA |
| | brain, hypothalamus, Huntington's, mw/CVA, 57M, NORM |
| | brain, temporal cortex, aw/CHF, 35M |
| | breast, mw/lobular CA, 58F, m/BRSTTUT03 |
| | colon, epithelium, 13F, RP |
| | colon, normal/pseudopolyp, Crohn, 16, 26M, pool, lg cDNA |
| | kidney, cortex, mw/renal cell CA, 65M, 5RP |
| | lung, mw/spindle cell carcinoid, 62F |
| | lymph node, 16mM |
| | microvascular, dermal, endothelial cells, 22F, Untx |
| | nose, nasal polyp, eosinophilia, aw/asthma, 78M |
| | nose, nasal polyps, pool, lg cDNA |
| | ovary, endometriosis, 24F |
| | placenta, neonatal, F, NORM, WM |
| | prostate stroma, fibroblasts, fetal, 26wM, untreated |
| | prostate, AH, mw/adenoCA, 58M |
| | skin, dermis, breast, fibroblasts, 31F, t/9CIS RA, lg cDNA, EF |
| | sm intestine, 8M |
| | spleen, aw/pancreas neuroendocrine CA, 65F |
| | spleen, fetal, WM |
| | synovium, knee, OA, 82F |
| | testis tumor, embryonal CA, 31M, 5RP |
| | thyroid, aw/CHF, 64F |
| | brain tumor, glioblastoma, pool, NORM, CGAP |
| | brain tumor, mixed types, pool, NORM, CGAP |
| | brain, allocortex, cingulate, aw/cholangioCA, 55F, RP |
| | breast tumor, adenoCA, 55F, m/BRSTNOT02 |
| | colon tumor, cecum, adenoCA, 70F |
| | ganglion, dorsal root, thoracic, aw/lymphoma, 32M, NORM |
| | ganglion, dorsal root, thoracic/lumbar, aw/lymphoma, 32M |
| | heart tumor, left atrium, myxoma, 43M |
| | kidney, cortex, mw/renal cell CA, 65M |
| | kidney, pool, SUB, 3′ CGAP |
| | lung, mw/adenoCA, aw/node, diaphragm mets, 63F |
| | mast cells, liver, fetal, 22w |
| | ovary, stromal hyperthecosis, mw/dermoid cyst, 45F, RP |
| | prostate, 32M, SUB, 3′ CGAP |
| | sm intestine, mw/carcinoid, aw/node mets, 59M, RP |
| | submandibular gland, aw/sialoadenitis, 49F, lg cDNA, EF |
| | uterus tumor, leiomyoma, 37F, 5RP |
| | uterus, myometrium, mw/leiomyoma, 43F |
| | Jurkat line, T-cell leukemia, M, t/PMA |
| | Jurkat line, T-cell leukemia, untreated, TIGR |
| | PBMC, 28-57M/F, pool, Untx-24 hr, FL-EN, EF |
| | T-lymphocytes, allogenic anergic, 40-50M, t/OKT3 3 day |
| | T-lymphocytes, allogenic, 40-50M, untreated |
| | UCB, derived dendritic cells, M, t/PMA, Iono-5 hr |
| | adenoid, inflamed, 3 |
| | adrenal gland, 17M |
| | adrenal gland, 8M |
| | adrenal glands, aw/anencephaly, fetal, 16wF, lg cDNA |
| | adrenal tumor, adrenal cortical CA, 49M, 5RP, EF |
| | adrenal tumor, cortical CA, aw/hilar mets, 52M, RP |
| | adrenal tumor, mets renal cell CA, 50M |
| | adrenal tumor, pheochromocytoma, 52F, EF |
| | bladder tumor, TC CA, 58M, m/BLADNOT09 |
| | bladder tumor, TC CA, 60M, m/BLADNOT05 |
| | bladder tumor, TC CA, 66M, m/BLADNOT06 |
| | bladder tumor, microscopic foci TC CA, 58M |
| | bone marrow, tibia, aw/mets alveolar rhabdomyoSAR, 16M, EF |
| | bone tumor, sacrum, giant cell tumor, 18F |
| | bone tumor/line, MG-63, osteoSAR/giant cell, M/F, pool, RP, EF |
| | brain tumor, frontal, meningioma, 50M |
| | brain tumor, frontal, meningioma, 61F |
| | brain tumor, frontal, mets hypernephroma, 58M |
| | brain tumor, meningioma, pool, 3′, CGAP |
| | brain, acute/chronic multiple sclerosis, pool |
| | brain, cerebellum, TIGR |
| | brain, temporal, gliosis, mw/epilepsy, 27M, lg cDNA |
| | brain, temporal, gliosis, mw/epilepsy, cerebral palsy, 27M |
| | breast tumor, adenoCA, 46F, m/BRSTNOT17 |
| | breast, 46, 60F, pool, NORM |
| | breast, NF changes, mw/ductal adenoCA, 40-57F, pool, lg cDNA |
| | breast, PF changes, 40F |
| | breast, PF changes, mw/adenoCA, 45F, m/BRSTTUT08 |
| | breast, PF changes, mw/adenoCA, 55F, m/BRSTTUT01 |
| | breast, PF changes, mw/adenoCA, intraductal CA, 43F |
| | breast, duct, mw/ductal CA, F, m/BRSTTUP04, 3′/5′ CGAP |
| | breast, mw/ductal CA, 43F, m/BRSTTUT16 |
| | breast, mw/ductal CA, CA in situ, aw/node mets, 62F |
| | breast, mw/ductal adenoCA, intraductal CA, aw/node mets, 57F |
| | chest wall, soft tissue, mw/adenoCA, aw/COPD, 63M |
| | colon tumor, adenoCA, 60M, m/COLNNOT07/08/09/11 |
| | colon tumor, adenoCA, M/F, pool, 3′ CGAP |
| | colon tumor, adenoCA, carcinoid, M/F, pool, NORM |
| | colon tumor, adenoCA, pool, NORM, 3′ CGAP |
| | colon tumor, ileocecum, Burkitt lymphoma, 29F, m/COLANOT03 |
| | colon tumor, villous adenoma, 3′ CGAP |
| | colon, cecum/descending, polyposis, polyp, M/F, pool, SUB |
| | colon, mixed tissues, 16M/13F, pool, NORM |
| | colon, mw/adenoCA, aw/COPD, 75M, m/COLNTUT02 |
| | colon, mw/adenoCA, aw/node mets, 60M, NORM, m/COLNTUT16 |
| | colon, normal/pseudopolyp, Crohn, 16, 26M, pool, lg/N |
| | colon, transverse, benign familial polyposis, 16M |
| | esophagus tumor, adenoCA, 61M, 5RP, EF |
| | esophagus tumor, squamous cell CA, 3′, CGAP |
| | fallopian tube tumor, endometrioid/serous adenoCA, 85F, 5RP, EF |
| | fat, mesentery, aw/diverticulosis, diverticulitis, 71M |
| | femoral artery, aw/chondroSAR, 68M |
| | fetus, 8-9w, pool, NORM, CGAP/WM/WN |
| | fibroblasts, senescent, NORM, WM/WN |
| | gallbladder, 25F, TIGR |
| | gallbladder, cholecystitis, cholelithiasis, 18F |
| | ganglion, dorsal root, cervical, aw/lymphoma, 32M |
| | germ cell tumor, pool, NORM, 3′ CGAP |
| | germ cell tumor, pool, SUB, 3′ CGAP |
| | heart, aorta, 17F |
| | kidney tumor, clear cell type cancer, pool, SUB, CGAP |
| | kidney, 8M |
| | liver tumor line, C3A, Hepatob, 15M, t/MCA, SUB |
| | liver, aw/Patau's, anencephaly, fetal, pool, lg cDNA |
| | liver, pool, NORM, EF |
| | lung tumor, adenoCA, 53M, m/LUNGNOT28 |
| | lung tumor, neuroendocrine carcinoid, pool, NORM, 3′ CGAP |
| | lung tumor, neuroendocrine carcinoid, pool, SUB, CGAP |
| | lung tumor, poorly differentiated, 3′ CGAP |
| | lung tumor, squamous cell CA, 69M, m/LUNGNOT15 |
| | lung tumor, squamous cell CA, pool, 3′, CGAP |
| | lung, 15F |
| | lung, 2M |
| | lung, 35F, 5RP |
| | lung, 72M, WM/WN |
| | lung, mw/adenoCA, 63M |
| | lung, mw/adenoCA, 66F |
| | lung, mw/adenoCA, COPD, 47M |
| | lung, mw/mets osteoSAR, aw/pleura mets, 58M |
| | lung, panacinar emphysema, mw/granuloma, 61M |
| | lung, right bronchus, nonasthmatic, 18-55 M/F, pool |
| | lymph node tumor, follicular lymphoma, 3′ CGAP |
| | lymph node, 11F |
| | lymph nodes, 14F |
| | lymphocytes, PBMC, M, 96-hr MLR |
| | lymphocytes, nonactivated Th1 cells |
| | mast cell line, HMC-1, leukemia, 52F, untreated |
| | meniscus, tibial, aw/mets alveolar rhabdomyoSAR, 16M |
| | mixed tissues, includes tumor, treated cells, pool, NORM, EF |
| | mixed, (melanocytes, uterus, fetal heart), SUB, CGAP/WM/WN |
| | muscle, forearm, mw/intramuscular hemangioma 38F |
| | neuroganglion tumor, ganglioneuroma, 9M |
| | nose, nasal polyps |
| | ovary tumor, dermoid cyst, 22F |
| | ovary, aw/leiomyoma, 47F |
| | ovary, aw/leiomyomata, 36F, NORM |
| | ovary, aw/menorrhagia, 47F, 5RP |
| | ovary, endometriosis; aw/leiomyomata, 39F, NORM, EF |
| | pancreas tumor, adenoCA, 3′ CGAP |
| | pancreas, fetal, 23wM |
| | pancreas, type I diabetes, 43F, RP |
| | penis tumor, squamous cell CA, 64M |
| | penis, mixed tissues, aw/urothelial CA, M, pool, NORM |
| | periph blood, B-lymphocytes, CLL, pool, NORM, 3′ CGAP |
| | periph blood, blast cells, AML, 58F |
| | periph blood, eosinophils, hypereosinophilia, 48M |
| | periph blood, granulocytes, M/F, t/GM-CSF |
| | periph blood, lymphocytes, non-adher PBMC, 24M |
| | periph blood, lymphocytes, non-adher PBMC, M/F, 24-hr MLR |
| | periph blood, macrophages, adher PBMC, M/F, MLR-24 hr |
| | periph blood, monocytes, 42F, t/IL-10, LPS |
| | periph blood, monocytes, 42F, t/IL-10, LPS, NORM |
| | periph blood, promonocyte line, THP-1, AML, t/5AZA |
| | periph blood, promonocyte line, THP-1, AML, untreated |
| | pituitary gland, aw/schizophrenia, COPD, 55M, NORM |
| | placenta, aw/hydrocephalus, fetal, 16w, 5RP |
| | prostate tumor, adenoCA, 59M, SUB, m/PROSNOST19 |
| | prostate tumor, adenoCA, 59M, SUB, m/PROSNOT19 |
| | prostate tumor, adenoCA, 65M, m/PROSNOT20 |
| | prostate tumor, adenoCA, 66M, m/PROSNOT15, PROSDIN01 |
| | prostate tumor, adenoCA, 69M, m/PROSNOT07 |
| | prostate, AH, mw/adenoCA, 55M, m/PROSTUT16 |
| | prostate, AH, mw/adenoCA, 66M |
| | prostate, AH, mw/adenoCA, 67M |
| | prostate, epithelium, PIN, mw/cancer, 45M, m/PROETUP02, CGAP |
| | seminal vesicle, aw/prostate adenoCA, 63M, 5RP |
| | skin, foreskin, melanocytes, M, NORM, WM/WN |
| | sm intestine tumor, ileum, mets ovarian adenoCA, 49F |
| | sm intestine, 31F, RP |
| | sm intestine, aw/Patau's syndrome, fetal, 20wM, 5RP |
| | sm intestine, fetal, 23wM, FL-EN, EF |
| | sm intestine, fetal, M, 5RP |
| | sm intestine, ileum, Crohn's, 26M |
| | sm intestine, ileum, mw/CUC, 25F |
| | spleen, 2M |
| | spleen, 8M |
| | spleen, ITP, 14M |
| | spleen, ITP, 8, 14M, pool, lg cDNA |
| | spleen, fetal, aw/hypoplastic left heart, 23wM, pool, AMP |
| | stomach tumor, adenoCA, poorly differentiated, 3′ CGAP |
| | stomach, aw/esophagus adenoCA, 61M, 5RP |
| | synovium, rheuA, 75M/56F, pool, NORM |
| | teratoCA line, NT2, Untx, NORM |
| | testis tumor, embryonal CA, 31M, EF |
| | testis, M, NORM, CGAP/WN |
| | thymus, aw/parathyroid adenoma, 21M |
| | thymus, aw/patent ductus arteriosus, 3M |
| | thymus, aw/patent ductus arteriosus, 3M, 5RP |
| | thymus, fetal, M |
| | thymus, hyperplasia, aw/myasthenia gravis, 16F |
| | thyroid, lymphocytic thyroiditis, mw/papillary CA, 30F |
| | thyroid, mw/follicular adenoma, 28F |
| | tonsil, B-lymphocytes, germinal, aw/tonsillitis, NORM, CGAP |
| | umb cord blood, T-lymphocytes, Th2 cells, untreated |
| | uterus tumor, endometrial adenoCA, F, pooled, 3′ CGAP |
| | uterus tumor, leiomyoma, 34F |
| | uterus tumor, serous papillary CA, F, pooled, 3′ CGAP |
| | uterus, F, NORM, CGAP/WM/WN |
| | uterus, endometrium, aw/cystocele, 38F |
| | uterus, endometrium, mw/cervical dysplasia, 32F |
| | uterus, endometrium, mw/leiomyoma, 29F, NORM |
| | uterus, myometrium, mw/adenoCA, 50F |
| | uterus, myometrium, mw/leiomyoma, 41F, NORM, m/UTRSTUT05 |
| | white blood cells, 27F |
| | whole blood, myeloid cells, CML, pool, 3′ CGAP |
| | Jurkat line, T-cell leukemia, M, Untx, 5C-FL, EF |
| | bladder, mw/TC CA, CA in situ, 60M, m/BLADTUT04 |
| | colon tumor, hepatic flexure, adenoCA, 55M, SUB, m/COLATMT01, EF |
| | ovary tumor, mucinous cystadenoCA, 43F, m/OVARNOT03 |
| | synovium, 75M, lg cDNA, EF |
| | thyroid, AH, mw/papillary CA, 22F, 5RP, EF |
| | uterus, myometrium, mw/adenoCA, 59F, RP |
| | brain, pineal gland, aw/AD, 68, 79F, pool, NORM |
| | brain, substantia nigra, aw/atherosclerosis, CHF, 81F, NORM |
| | breast tumor, adenoCA, 45F, m/BRSTNOT09 |
| | colon, appendix, aw/leiomyomata, 37F |
| | colon, mw/adenoCA, aw/node mets, 60M, m/COLNTUT16 |
| | heart, 44M, NORM |
| | liver/spleen, fetal, 20wM, NORM, CGAP/WM/WN |
| | lung tumor, adenoCA, 47M |
| | prostate tumor, adenoCA, 58, 61, 66, 68M, pool, SUB |
| | synovium, wrist, rheuA, 56F |
| | testis, 16M |
| | testis, 26M |
| | testis, aw/cirrhosis, 37M |
| | uterus, cervix, cervicitis, mw/leiomyoma, 29F, lg cDNA, EF |
| | lung, pleura, aw/mets leiomyoSAR to lung, 58F, lg cDNA |
| | bladder, mixed, TC CA/Nrml, 58, 72M, pool, NORM, EF |
| | brain tumor, benign meningioma, 35F |
| | brain, neocortex, parietal, aw/cholangioCA, 55F, RP |
| | breast tumor, ductal CA, 68F |
| | colon, ascending, CUC, 32M |
| | epiglottis, aw/papillary thyroid CA, 71M |
| | fallopian tube tumor, endometrioid/serous adenoCA, 85F, RP |
| | liver tumor, mets neuroendocrine CA, 72M, 5RP, EF |
| | lung tumor, squamous CA, 50M |
| | lung tumor, squamous cell CA, 65F |
| | lung, aw/anencephaly, fetal, 20wF |
| | lymph node tumor, Hodgkin's/mets adenoCA, F, pool, lg cDNA |
| | ovary, aw/leiomyomata, 36F |
| | ovary, mw/follicular cysts, 28F |
| | skin, leg, erythema nodosum |
| | stomach, fetal, 18wM |
| | thymus, 3M, NORM |
| | uterus, soft tissue, mw/leiomyomata, 33F |
| | lymph nodes, 16mM, NORM |
| | probable astrocytes, M/F, untreated |
| | skin, breast, 26F |
| | spleen, fetal, 23wM |
| | thymus, 3M |
| | tonsil, lymphoid hyperplasia, 6M |
| | Eye, normal, pigmented retinal epithelium |
| | B-cell precursor |
| #40980: | brain tumor, benign meningioma, 35F, 5RP |
| | mixed tissues, includes tumor, 8-69M/F, pool, NORM, EF |
| | mixed, (melanocytes, uterus, fetal heart), SUB, CGAP/WM/WN |
| | ovary tumor, mets colon adenoCA, 58F |
| | sm intestine, ileum, chronic inflammation, 29F, 5RP, EF |
| | sm intestine, ileum, mw/carcinoid, 30F |
| | spleen tumor, malignant lymphoma, 28M, 5RP |
| | mixed tissues, includes tumor, 20w-72M/F, pool, NORM, EF |
| | penis, mixed tissues, aw/urothelial CA, M, pool, NORM |
| | brain, hippocampus, AD |
| | B-Cells, Raji/Daudi tumor line, Burkitt, pool, Untx, FL-EN, EF |
| | UCB, derived dendritic cells, M, t/PMA, Iono-5 hr |
| | UCB, derived dendritic cells, pool, untreated/treated, NORM |
| | adrenal gland, mw/pheochromocytoma, 43F, m/ADRETUT07 |
| | adrenal tumor, adrenal cortical CA, 49M, 5RP, EF |
| | adrenal tumor, cortical CA, aw/hilar mets, 52M, RP |
| | aorta, endothelial cells, 33F, treated |
| | bladder tumor, microscopic foci TC CA, 58M |
| | bladder, mw/TC CA, 80F, m/BLADTUT02 |
| | bladder, mw/TC CA, CA in situ, 60M, m/BLADTUT04 |
| | bladder, mw/TC CA, aw/prostate TC CA, 66M, m/BLADTUT05 |
| | bone marrow line, SH-SY5Y, neuroblastoma, 4F, t/6-OHDA, 5RP, EF |
| | bone marrow, 16-70M/F, RP |
| | bone tumor, rib, mets osteoSAR, 16M |
| | brain tumor, anaplastic oligodendroglioma, pool, NORM, CGAP |
| | brain tumor, frontal, astrocytoma, 17F |
| | brain tumor, frontal, astrocytoma, 47M |
| | brain tumor, frontal, neuronal neoplasm, 32M |
| | brain tumor, glioblastoma, pool, NORM, CGAP |
| | brain tumor, meningioma, 36M |
| | brain tumor, oligodendroglioma, 44M, WN |
| | brain tumor, posterior fossa, meningioma, 70M |
| | brain, allocortex, cingulate, aw/cholangioCA, 55F, RP |
| | brain, allocortex/neocortex, cingulate, aw/CA, 55F, RP |
| | brain, archaeocortex, hippocampus, aw/cholangioCA, 55F, RP |
| | brain, astrocytes, fetal, 22wF, untreated |
| | brain, aw/hypoplastic left heart, fetal, 23wM |
| | brain, aw/hypoplastic left heart, fetal, 23wM, 5RP |
| | brain, aw/hypoplastic left heart, fetal, 23wM, NORM |
| | brain, aw/hypoplastic left heart, fetal, 23wM, lg cDNA |
| | brain, aw/muscular atrophy, 3mF, NORM, GEXP |
| | brain, caudate/putamen/nucleus accumbens, aw/CHF, 35M |
| | brain, cerebellum, Huntington's, mw/CVA, 57M |
| | brain, cerebellum, TIGR |
| | brain, cerebellum, aw/COPD, 69M |
| | brain, cerebellum, aw/COPD, left ventricular failure, 70M |
| | brain, choroid plexus, hemorrhage, 44M |
| | brain, dentate nucleus, aw/CHF, 35M, lg cDNA |
| | brain, fetal, 23wM |
| | brain, frontal cortex, M, pool, WN |
| | brain, frontal, Huntington's, mw/CVA, 57M |
| | brain, frontal, polymicrogyria, gliosis, 5M |
| | brain, globus pallidus/substantia innominata, aw/CHF, 35M |
| | brain, hippocampus, 2F, SUB, 3′ TIGR |
| | brain, hippocampus, mw/intracranial hemorrhage, 72F, NORM |
| | brain, hypothalamus, Huntington's, mw/CVA, 57M |
| | brain, hypothalamus, Huntington's, mw/CVA, 57M, NORM |
| | brain, infant, 10wF, NORM, WM |
| | brain, medulla, Huntington's, mw/CVA, 57M |
| | brain, mixed tissues, aw/CHF, 35M, pool, lg/N |
| | brain, mixed tissues, aw/aortic aneurysm, 45F |
| | brain, mixed tissues, aw/cholangioCA, 55F, RP |
| | brain, mixed tissues, gliosis, 27, 35M, pool, lg cDNA |
| | brain, mw/oligoastrocytoma, epilepsy, 26M |
| | brain, mw/oligoastrocytoma, epilepsy, 26M, NORM |
| | brain, pons, Huntington's, mw/CVA, 57M |
| | brain, sensory-motor cortex, aw/CHF, 35M |
| | brain, striatum/globus pallidus/putamen, aw/CHF, 81F, RP |
| | brain, temporal cortex, aw/aortic aneurysm, 45F |
| | brain, temporal, gliosis, mw/epilepsy, 27M, lg cDNA |
| | brain, temporal, gliosis, mw/epilepsy, cerebral palsy, 27M |
| | brain, temporal, mw/neuroepithelial tumor, epilepsy, 45M |
| | breast tumor, adenoCA, 46F, m/BRSTNOT17 |
| | breast tumor, adenoCA, 46F, m/BRSTNOT33, EF |
| | breast tumor, adenoCA, 62F, m/BRSTNOT14 |
| | breast tumor, ductal CA, 43F, m/BRSTTMT01 |
| | breast tumor, ductal, F, pool, NORM, 3′/5′ CGAP |
| | breast tumor, lobular CA, 58F, m/BRSTNOT05 |
| | breast, F, NORM, WM |
| | breast, PF changes, mw/adenoCA, 55F, m/BRSTTUT01 |
| | breast, mw/ductal CA, CA in situ, aw/node mets, 62F |
| | breast, mw/lobular CA, 67F |
| | bronchial epithelium line, NHBE, 54M, Untx |
| | cartilage, OA |
| | cartilage, knee, chondrocytes, M/F, t/IL-1 |
| | clavicle, osteoblasts, 40M, untreated, lg cDNA, EF |
| | colon tumor, adenoCA, 75M, m/COLNNOT01 |
| | colon tumor, adenoCA, NORM, 3′ CGAP |
| | colon tumor, adenoCA, pool, NORM, 3′ CGAP |
| | colon tumor, cecum, adenoCA, 45F |
| | colon, 25M, WN |
| | colon, ascending, CUC, 74M, EF |
| | colon, aw/anencephaly, fetal, 20wF |
| | colon, cecum/descending, polyposis, polyp, M/F, pool, SUB |
| | colon, descending, CUC, 28M, 5RP |
| | colon, epithelium, 13F, RP |
| | colon, mw/adenoCA, aw/node mets, 60M, m/COLNTUT16 |
| | colon, sigmoid, mw/adenoCA, aw/node mets, 62M, m/COLNTUT03 |
| | colon, transverse, Crohn's, 26M |
| | fat, breast, aw/fibrosis, 38F |
| | fat, mesentery, aw/diverticulosis, diverticulitis, 71M |
| | fetus, 8-9w, pool, NORM, CGAP/WM/WN |
| | fibroblasts, senescent, NORM, WM/WN |
| | gallbladder, cholecystitis, cholelithiasis, 53F |
| | ganglion, dorsal root, thoracic, aw/lymphoma, 32M, NORM |
| | germ cell tumor, pool, SUB, 3′ CGAP |
| | heart, 44M, NORM |
| | heart, aorta, 12F |
| | heart, coronary artery, CAD, 46M |
| | heart, fetal, 19w, NORM, CGAP/WM/WN |
| | heart, fetal, 8-10w, pool, BI |
| | heart, left ventricle, Pompe's, 7mM, RP |
| | heart, right atrium/muscle wall, Pompe's, 7mM, RP |
| | iliac artery, endothelial cells, F, t/1% oxygen 24 hr |
| | kidney tumor, Wilms', pool, WM/WN |
| | kidney, aw/anencephaly, fetal, 17wF |
| | kidney, interstitial nephritis, aw/bladder TC CA, 63M, 5RP |
| | kidney, pool, NORM, 3′ CGAP |
| | kidney, right/left, aw/Patau's, fetal, 20wM, pool, lg cDNA |
| | liver tumor line, C3A, Hepatob, 15M, t/APAP-48 hr |
| | liver tumor, mets neuroendocrine CA, 72M, 5RP, EF |
| | liver, aw/Patau's, anencephaly, fetal, pool, lg cDNA |
| | liver, pool, NORM, EF |
| | liver/spleen, fetal, 20wM, NORM, WM |
| | lung tumor, myxoid lipoSAR, 65F |
| | lung tumor, neuroendocrine carcinoid, pool, NORM, 3′ CGAP |
| | lung tumor, neuroendocrine carcinoid, pool, SUB, CGAP |
| | lung tumor, squamous cell CA, 65F |
| | lung, 35F, 5RP |
| | lung, 72M, WM/WN |
| | lung, NORM |
| | lung, asthma, 17M |
| | lung, aw/anencephaly, fetal, 20wF |
| | lung, idiopathic pulmonary disease, SUB |
| | lung, mw/adenoCA, 66F |
| | lung, mw/adenoCA, aw/node, diaphragm mets, 63F |
| | lung, panacinar emphysema, mw/granuloma, 61M |
| | lymph node tumor, mets melanoma, 3′, CGAP |
| | lymph node tumor, mets squamous cell CA, aw/tongue CA, 5RP |
| | lymphocytes, PBMC, M, 96-hr MLR |
| | lymphocytes, nonactivated Th1 cells |
| | mammary, epithelial cells, 21F, untreated, NORM |
| | mast cells, liver, fetal, 22w |
| | microvascular, dermal, endothelial cells, 22F, Untx |
| | mixed tissues, fetal lung, testis, B-cell, SUB, 3′ CGAP/WN |
| | mixed tissues, myometrium, smooth muscle cells, 57M/41F, B |
| | multiple sclerosis, 46M, NORM, WM/WN |
| | muscle, forearm, mw/intramuscular hemangioma 38F |
| | neuroganglion tumor, ganglioneuroma, 9M |
| | nose, nasal polyps, aw/asthma, 78M, pool, NORM |
| | nose, nasal polyps, pool, lg cDNA |
| | ovary, aw/leiomyomata, 36F, NORM |
| | ovary, stromal hyperthecosis, mw/dermoid cyst, 45F, RP |
| | pancreas tumor, adenoCA, 3′ CGAP |
| | pancreas, fetal, 23wM |
| | pancreas, type I diabetes, 43F, RP |
| | pancreatic tumor, TIGR |
| | paraganglion tumor, paraganglioma, aw/renal cell CA, 46M |
| | periph blood, B-lymphocytes, CLL, pool, NORM, 3′ CGAP |
| | periph blood, eosinophils, t/IL-5 |
| | periph blood, promonocyte line, THP-1, AML, t/5AZA |
| | periph blood, promonocyte line, THP-1, AML, t/5AZA, SUB |
| | pituitary gland, aw/AD, mets adenoCA, 70F, RP |
| | pituitary gland, aw/schizophrenia, COPD, 55M, NORM |
| | placenta, neonatal, F, NORM, WM |
| | probable astrocytes, M/F, untreated |
| | probable cartilage, knee, chondrocytes, M/F, t/IL-1 |
| | prostate tumor, TC CA, 66M, EF |
| | prostate tumor, adenoCA, 59M, SUB, m/PROSNOT19 |
| | prostate tumor, adenoCA, 65M, m/PROSNOT20 |
| | prostate, 28M, NORM |
| | prostate, 32M, SUB, 3′ CGAP |
| | prostate, AH, mw/adenoCA, 48-73M, pool, lg cDNA |
| | prostate, AH, mw/adenoCA, 65M |
| | prostate, AH, mw/adenoCA, 66M, m/PROSTUT10 |
| | prostate, AH, mw/adenoCA, M, m/PROSTUT13, PROSTUS08/19/20/25 |
| | prostate, AH, mw/adenoCA, node mets, 55M, lg/N, m/PROSTUT16 |
| | prostate, BPH, mw/adenoCA, PIN, 59M |
| | prostate, epithelium, mw/cancer, PIN, 45M, m/PROETUP02, CGAP |
| | prostate, mw/adenoCA, 65M |
| | seminal vesicle, aw/adenoCA, 56M |
| | seminal vesicle, aw/adenoCA, 58M |
| | seminal vesicle, aw/prostate adenoCA, 63M, 5RP |
| | skin, breast, 26F |
| | skin, dermis, breast, fibroblasts, 31F, t/9CIS RA-20 hr |
| | sm intestine, aw/stomach ulcer, 49F, 5RP |
| | sm intestine, ileum, Crohn's, 18F |
| | spinal cord, aw/renal failure, 71M, NORM |
| | spleen, fetal, 23wM |
| | stomach tumor, adenoCA, poorly differentiated, 3′ CGAP |
| | stomach, fetal, 18wM |
| | synovium, wrist, rheuA, 62F |
| | teratoCA line, NT2, t/5AZA-3d |
| | teratoCA line, NT2, t/5AZA-3d, SUB |
| | teratoCA line, hNT2, t/RA |
| | teratoCA line, hNT2, t/RA + MI, WM |
| | teratoCA line, hNT2, untreated, WM/WN |
| | testis tumor, embryonal CA, 31M, EF |
| | testis tumor, seminoma, 45M |
| | testis, M, NORM, CGAP/WN |
| | thymus, 3M |
| | thymus, aw/anencephaly, fetal, 17wF |
| | thymus, hyperplasia, aw/myasthenia gravis, 16F |
| | thyroid, mw/medullary, papillary CA, node mets, 56M |
| | tibia, periosteum, mw/osteoSAR, osteogenesis imperfecta, 20M |
| | uterus, aw/ovarian follicular cysts, 34F |
| | uterus, cervix, 40F |
| | uterus, endometrium, 32F |
| | uterus, endometrium, F, pool |
| | uterus, myometrium, mw/adenoCA, 59F, RP |
| | uterus, myometrium, mw/leiomyoma, 41F, m/UTRSTUT05 |
| | brain |

List of tissues where gene-supporting ESTs were found (part 2)

| | |
|---|---|
| #41437: | teratoCA line, hNT2, t/mouse leptin, RA |
| | CML precursor line, K-562, 53F, t/5AZA-72 hr |
| | bladder tumor, TC CA, 66M, m/BLADNOT06 |
| | brain, aw/hypoplastic left heart, fetal, 23wM, NORM |
| | periph blood, promonocyte line, THP-1, AML, t/5AZA, SUB |
| | sm intestine, 31F, RP |
| | thymus, fetal, M |
| | brain, hypothalamus, Huntington's, mw/CVA, 57M |
| | germ cell tumor, pool, SUB, 3′ CGAP |
| | pituitary gland, aw/schizophrenia, COPD, 55M, NORM |
| | testis tumor, embryonal CA, 31M, 5RP |
| | colon tumor, adenoCA, NORM, SUB, CGAP |
| | brain, fetal, 23wM, 5C-RP |
| | Jurkat line, T-cell leukemia, M, untreated |
| | Jurkat line, T-cell leukemia, untreated, TIGR |
| | T-lymphocytes, allogenic anergic, 40-50M, t/OKT3 3 day |
| | adrenal glands, aw/anencephaly, fetal, 16wF, lg cDNA |
| | aortic smooth muscle line, M |
| | bladder tumor, TC CA, 60M, m/BLADNOT05 |
| | bladder tumor, TC CA, 80F, m/BLADNOT03 |
| | bladder, mw/TC CA, aw/node mets, 58M, m/BLADTUT03 |
| | bone marrow line, SH-SY5Y, neuroblastoma, 4F, t/6-OHDA, 5RP, EF |
| | bone marrow tumor line, SH-SY5Y, pool, Untx/t/6OHDA, NORM, EF |
| | brain tumor, anaplastic oligodendroglioma, pool, NORM, CGAP |
| | brain tumor, frontal, astrocytoma, 47M |
| | brain tumor, glioblastoma, pool, NORM, CGAP |
| | brain, archaeocortex, hippocampus, aw/cholangioCA, 55F, RP |
| | brain, astrocytes, fetal, 22wF, t/TNF, IL-1 24 hr |
| | brain, aw/hypoplastic left heart, fetal, 23wM, lg cDNA |
| | brain, aw/muscular atrophy, 3mF, NORM, GEXP |
| | brain, cerebellum, TIGR |
| | brain, hippocampus, aw/CHF, 35M |
| | brain, hypothalamus, Huntington's, mw/CVA, 57M, NORM |
| | brain, infant, 10wF, NORM, WM |
| | brain, parietal cortex, aw/CHF, 35M, AMP/N |
| | brain, pineal gland, aw/AD, 68, 79F, pool, NORM |
| | brain, temporal cortex, aw/CHF, 35M |
| | brain, temporal cortex, aw/aortic aneurysm, 45F |
| | breast tumor, adenoCA, 46F, m/BRSTNOT17 |
| | breast tumor, adenoCA, 55F, m/BRSTNOT02 |
| | breast tumor, ductal CA, 65F |
| | breast, NF breast disease, 46F |
| | breast, mw/ductal CA, 43F, m/BRSTTUT16 |
| | breast, mw/ductal adenoCA, 46F, m/BRSTTUS08, BRSTTUT13 |
| | breast, mw/ductal adenoCA, CA in situ, 62F, m/BRSTTUT14 |
| | breast, mw/neoplasm, 36F |
| | bronchial epithelium line, NHBE, 54M, Untx |
| | colon tumor line, KM12C, CA, Untx, TIGR |
| | colon tumor, adenoCA, 3′, CGAP |
| | colon tumor, adenoCA, 60M, m/COLNNOT07/08/09/11 |
| | colon tumor, adenoCA, pool, NORM, 3′ CGAP |
| | colon tumor, adenoCA, pool, NORM, 3′/5′ CGAP |
| | colon tumor, ileocecum, Burkitt lymphoma, 29F, m/COLANOT03 |
| | colon, aw/anencephaly, fetal, 20wF |
| | colon, cecum/descending, polyposis, polyp, M/F, pool, NORM |
| | coronary artery, smooth muscle cells, 3M, NORM |
| | eye, retina, M/F, TIGR |
| | ganglion, dorsal root, cervical, aw/lymphoma, 32M, NORM, EF |
| | ganglion, dorsal root, thoracic, aw/lymphoma, 32M |
| | germ cell tumor, yolk sac, 3′ CGAP |
| | heart, aorta, 39M, lg cDNA, EF |
| | heart, aorta, aw/cerebral agenesis, 27F |
| | heart, fetal, 19w, NORM, CGAP/WM/WN |
| | kidney epithelial transf embryo line, 293-EBNA, serum starv |
| | kidney epithelial transf embryo line, 293-EBNA, t/5AZA |
| | kidney epithelial transf embryo line, 293-EBNA, t/5AZA, SUB |
| | kidney, 8M |
| | kidney, cortex, mw/renal cell CA, 65M, 5RP |
| | kidney, interstitial nephritis, aw/bladder TC CA, 63M, 5RP |
| | kidney, pool, NORM, 3′ CGAP |
| | kidney, pool, SUB, 3′ CGAP |
| | liver tumor, mets neuroendocrine CA, 62F, m/ LIVRTMR01 |
| | liver, aw/Patau's, anencephaly, fetal, pool, lg cDNA |
| | liver/spleen, fetal, 20wM, NORM, WM |
| | lung tumor, neuroendocrine carcinoid, pool, SUB, CGAP |
| | lung tumor, poorly differentiated, 3′ CGAP |
| | lung tumor, squamous cell CA, 57M |
| | lung, 15F |
| | lung, NORM |
| | lung, asthma, 8F |
| | lung, aw/Patau's, fetal, 20wM, lg cDNA |
| | lung, fetal, 19w, NORM, CGAP/WM/WN |
| | lung, mw/adenoCA, 63M |
| | lung, mw/mets osteoSAR, aw/pleura mets, 58M, NORM |
| | lymphocytes, PBMC, M, 96-hr MLR |
| | mesentery tumor, sigmoid, mets mixed-mullerian tumor, 61F |
| | microvascular, dermal, endothelial cells, neonatal, M |
| | mixed tissues, includes tumor, 20w-72M/F, pool, NORM, EF |
| | mixed tissues, includes tumor, SUB, 3′ CGAP |
| | mixed tissues, includes tumor, treated cells, pool, NORM, EF |
| | mixed tissues, myometrium, smooth muscle cells, 57M/41F, B |
| | mixed, (melanocytes, uterus, fetal heart), SUB, CGAP/WM/WN |
| | multiple sclerosis, 46M, NORM, WM/WN |
| | muscle, thigh, ALS, 74F, NORM |
| | ovary tumor, mets colon adenoCA, 58F |
| | ovary tumor, mixed types, pooled, F, 3′ CGAP |
| | ovary tumor, seroanaplastic CA, 52F |
| | ovary, aw/cardiomyopathy, 59F, NORM |
| | ovary, aw/leiomyomata, 36F, NORM |
| | ovary, epithelial cell line, pool, F, 3′, CGAP |
| | pancreas, 17F, NORM |
| | pancreatic tumor line, adenoCA, untreated, WM/WN |
| | pancreatic tumor, anaplastic CA, 45F |
| | parathyroid, hyperplasia, 69F |
| | periph blood, B-lymphocytes, CLL, pool, NORM, 3′ CGAP |
| | periph blood, eosinophils, nonallergic, M/F |
| | periph blood, macrophages, adher PBMC, M/F, 48-hr MLR |
| | periph blood, macrophages, adher PBMC, M/F, 72-hr MLR |
| | periph blood, macrophages, adher PBMC, M/F, MLR-24 hr |
| | peritoneum tumor, neuroendocrine CA, 66F |
| | prostate tumor, adenoCA, 59M, SUB, m/PROSNOST19 |
| | prostate, 28M |
| | renal vein, smooth muscle cells, 57M, Untx |
| | seminal vesicle, aw/prostate adenoCA, 63M, 5RP |
| | skin, epidermal breast keratinocyte line, NHEK, 30F, Untx |
| | sm intestine, 13M |
| | sm intestine, aw/Patau's syndrome, fetal, 20wM, 5RP |
| | sm intestine, aw/stomach ulcer, 49F, 5RP |
| | sm intestine, fetal, 20wF |
| | sm intestine, fetal, M, RP, EF |
| | soft tissue tumor, spinal schwannoma, 35M |
| | soft tissue tumor, thigh, mets myxoid lipoSAR, 34F |
| | spleen, 2M |
| | spleen, aw/hypoplastic left heart, fetal, 23wM, FL-EN, EF |
| | spleen, aw/pancreas neuroendocrine CA, 65F |
| | stomach tumor, adenoCA, poorly differentiated, 3′ CGAP |
| | stomach, aw/esophagus adenoCA, 61M, 5RP |
| | stomach, gastritis, mw/adenoCA, node mets, 76M, RP |
| | teratoCA line, NT2, t/cytokines, 5C-FL, EF |
| | teratoCA line, hNT2, untreated |
| | testis, 10-61M, pool, lg cDNA |
| | testis, 16M, NORM |
| | testis, 26M |
| | testis, M, NORM, CGAP/WN |
| | testis, aw/cirrhosis, 37M |
| | tonsil, B-lymphocytes, germinal, aw/tonsillitis, NORM, CGAP |
| | unspecified tissue, CNRS |
| | uterus tumor, leiomyoma, 41F |
| | uterus tumor, leiomyomata/adenosquamousCA, F, pool, lg cDNA, EF |
| | uterus, F, NORM, CGAP/WM/WN |
| | uterus, aw/ovarian follicular cysts, 34F |
| | uterus, endometrium, mw/cervicitis, leiomyoma, pool, lg cDNA |
| | uterus, mw/leiomyoma, aw/colon adenoCA, 45F |
| | uterus, mw/leiomyomata, 49, 55F, pool, lg cDNA, EF |
| | uterus, myometrium, mw/adenoCA, 50F |
| | uterus, myometrium, mw/multiple leiomyomata, 45F |
| | aorta, adventitia, 48M |
| | bone marrow tumor line, SH-SY5Y, neuroblastoma, 4F, t/RA |
| | brain line, fetal, 17-18wF, RP, 3′ TIGR |
| | brain tumor, frontal, oligoastrocytoma, 50F |
| | brain, amygdala/entorhinal cortex, aw/CA, 55F, RP, EF |
| | brain, astrocytes, fetal, 22wF, untreated |
| | brain, aw/hypoplastic left heart, fetal, 23wM, 5RP |
| | brain, frontal cortex, aw/CHF, 35M |
| | brain, frontal, Huntington's, mw/CVA, 57M |
| | brain, infant, F, TIGR |
| | ganglion, dorsal root, 36M, CGAP |
| | lung tumor, pleura, mets uterine leiomyoSAR, 55F |
| | lung, asthma, 10M |
| | lymph node, 11F |
| | muscle, gluteal, mw/clear cell SAR, 43F |
| | muscle, thigh, aw/lipoSAR, 64F |
| | muscle, thigh, mw/lipoSAR, 58M, RP |
| | nose, nasal polyp, eosinophilia, aw/asthma, 78M |
| | nose, nasal polyps, lg cDNA, EF |
| | olfactory bulb, aw/CA, 39-85M/F, pool, PlyAT, EF |
| | pancreas tumor, adenoCA, 3′ CGAP |
| | parotid gland, mw/Warthin's tumor, 70M |
| | prostate tumor, TC CA, 66M, EF |
| | prostate tumor, adenoCA, 59M, SUB, m/PROSNOT19 |
| | prostate, epithelial cells, 17M, untreated |
| | sm intestine, ileum, 4F |
| | stomach tumor, adenoCA, 52M, m/STOMNOT02 |
| | teratoCA line, hNT2, t/RA |
| | teratoCA line, hNT2, t/RA + MI, WM/WN |
| | testis tumor, seminoma, 45M |
| | uterus tumor, endometrial adenoCA, F, pooled, 3′ CGAP |
| | uterus, endometrium, mw/cervical dysplasia, 32F, 5RP |
| | myeloblast (KG-1) |
| | placenta, choriocarcinoma |
| #33751: | Jurkat line, T-cell leukemia, M, t/PMA-30 min, NORM, EF |
| | cervical tumor line, HeLa S3, adenoCA, 31F, untreated, WM/WN |
| | mixed tumor, Wilm's/brain mets, pool, 3′ CGAP |
| | prostate, epithelial cells, 17M, untreated |
| | skin, leg, erythema nodosum |
| | uterus, cervix, 40F |
| | brain tumor line, DU 145, mets prostate CA, 69M, Untx, 5C-FL, EF |
| | lung tumor, CA, pool, LICR, EF |
| | lung tumor, squamous cell CA, 68M, 5RP |
| | penis, corpus cavernosum, mw/CA, 53M |
| | placenta, aw/hydrocephalus, fetal, 16w/18wM, pool, RP |
| | sm intestine, ileum, Crohn's, 26M |
| | adipose, abdominal, aw/morbid obesity, 68F |
| | adrenal tumor, pheochromocytoma, 43F, m/ADRENOT11 |
| | amnion, pool, LICR, EF |
| | bladder tumor line, CA/TC CA/papilloma, pool, MGC, EF |
| | bladder, mw/TC CA, 80F, m/BLADTUT02 |
| | bone, rib, aw/Patau's, fetal, 20wM |
| | brain tumor, benign meningioma, 35F, 5RP |
| | brain tumor, frontal, meningioma, 61F |
| | brain tumor, frontal/parietal, meningioma, 76F |
| | brain tumor, meningioma, 36M |
| | brain tumor, meningioma, pool, 3′, CGAP |
| | brain, astrocytes, fetal, 22wF, t/TNF, IL-1 24 hr |
| | brain, aw/hypoplastic left heart, fetal, 23wM |
| | brain, choroid plexus, Huntington's, mw/CVA, 57M |
| | brain, pineal gland, aw/AD, 68, 79F, pool, NORM |
| | breast tumor line, Hs 578T, ductal CA, 74F, t/EGF-8 hr, 5C-FL, EF |
| | breast tumor line, T-47D, ductal CA, 54F, 5C-FL, EF |
| | breast tumor line, T-47D, ductal CA, 54F, Untx, NORM, EF |
| | breast tumor, adenoCA, 45F, m/BRSTNOT09 |
| | breast tumor, lobular CA, 58F, m/BRSTNOT05 |
| | breast, 46F |
| | breast, PF breast disease, 57F |
| | breast, mw/ductal CA, CA in situ, aw/node mets, 62F |
| | breast, mw/ductal adenoCA, intraductal CA, aw/node mets, 57F |
| | breast, mw/lobular CA, 58F, m/BRSTTUT03 |
| | bronchial epithelium line, NHBE, 54M, Untx, NORM |
| | colon tumor, juvenile granulosa cell, 3′ CGAP |
| | colon, mw/adenoCA, aw/node mets, 60M, m/COLNTUT16 |
| | colon, normal/pseudopolyp, Crohn, 16, 26M, pool, lg cDNA |
| | colon, transverse, benign familial polyposis, 16M |
| | eye, corneal fibroblasts primary line, 76, untreated |
| | fat, mesentery, aw/diverticulosis, diverticulitis, 71M |
| | fetus, 7-8w, pool, AMP, EF |
| | fetus, 8-9w, pool, NORM, CGAP/WM/WN |
| | gallbladder, cholecystitis, cholelithiasis, 18F |
| | ganglion, dorsal root, thoracic, aw/lymphoma, 32M, NORM |
| | germ cell tumor, pool, NORM, 3′ CGAP |
| | heart coronary artery endothelial cells, 3M, untreated, NORM |
| | heart, aorta, 10M |
| | heart, coronary artery, CAD, 46M |
| | heart, fetal, 18wM |
| | heart, fetal, 19w, NORM, CGAP/WM/WN |
| | heart, left ventricle, 51F |
| | heart, left ventricle, mw/myocardial infarction, 56M |
| | heart, mixed, hypoplastic/aw/patau's, fetal, pool, lg cDNA, EF |
| | heart, right atrium/muscle wall, Pompe's, 7mM, RP |
| | iliac artery, endothelial cells, F, t/TNF, IL-1 20 hr |
| | kidney tumor, CA, pool, LICR, EF |
| | kidney tumor, clear cell type cancer, pool, SUB, CGAP |
| | kidney tumor, renal cell CA, 65M m/KIDNNOT19 |
| | kidney tumor, renal cell CA, pool, 3′/5′ CGAP |
| | kidney, 2dF |
| | kidney, 64F |
| | kidney, MGC, EF |
| | kidney, aw/anencephaly, fetal, 17wF |
| | kidney, aw/hypoplastic left heart, fetal, 23wM, 5RP |
| | kidney, cortex, mw/renal cell CA, 65M |
| | kidney, cortex, mw/renal cell CA, 65M, 5RP |
| | kidney, fetal, TIGR |
| | kidney, medulla/cortex, mw/renal cell CA, 53F, m/KIDNTUT16 |
| | kidney, mw/renal cell CA, 65M, m/KIDNTUT15 |
| | kidney, pool, NORM, 3′ CGAP |
| | kidney, pool, SUB, 3′ CGAP |
| | liver tumor line, C3A, Hepatob, 15M, t/MCA-48 hr, SUB |
| | liver, 49M, WM |
| | liver, pool, NORM, EF |
| | liver/spleen, fetal, 20wM, NORM, CGAP/WM/WN |
| | liver/spleen, fetal, 20wM, NORM, WM |
| | lung tumor, adenoCA, 63M |
| | lung tumor, squamous CA, 50M |
| | lung tumor, squamous cell CA, 68M |
| | lung, MGC, EF |
| | lung, NORM |
| | lung, asthma, 15M |
| | lung, asthma, 17M |
| | lung, aw/Patau's, fetal, 20wM |
| | lung, aw/Patau's, fetal, 20wM, lg cDNA |
| | lung, fetal, 19w, NORM, CGAP/WM/WN |
| | lung, fetal, M, RP |
| | lung, pleura, aw/mets leiomyoSAR to lung, 58F, lg cDNA |
| | lymph node, necrotic, aw/lung squamous cell CA, 67M |
| | microvascular, dermal, endothelial cells, 22F, t/bFGF, EF |
| | mixed tissue tumor, head/neck, CA, pool, LICR, EF |
| | mixed tissue, head/neck, pool, LICR, EF |
| | mixed tissues, includes tumor, 20w-72M/F, pool, NORM, EF |
| | mixed tissues, includes tumor, 29-69F, pool, NORM, EF |
| | mixed tissues, includes tumor, M/F, pool, NORM, EF |
| | mixed tissues, includes tumor, SUB, 3′ CGAP |
| | mixed tissues, includes tumor, pool, SUB, 3′ CGAP |
| | mixed, (melanocytes, uterus, fetal heart), SUB, CGAP/WM/WN |
| | muscle, forearm, mw/intramuscular hemangioma 38F |
| | muscle, psoas, 12M |
| | muscle, thigh, mw/lipoSAR, 58M, RP |
| | nervous tissue tumor, CA, pool, LICR, EF |
| | nervous tissue, pool, LICR, EF |
| | ovary, aw/cardiomyopathy, 59F |
| | ovary, endometriosis, aw/leiomyomata, 39F |
| | pancreas tumor, adenoCA, 3′ CGAP |
| | pancreas, 29M |
| | pancreas, 2M |
| | penis, corpora cavernosa, M |
| | penis, corpora cavernosa, aw/scrotal urothelial CA, M |
| | placenta, MGC, EF |
| | placenta, aw/hydrocephalus, fetal, 16w, 5RP |
| | placenta, aw/hydrocephalus, fetal, 16w, RP |
| | placenta, aw/hydrocephalus, fetal, 16w, lg cDNA |
| | placenta, fetal, 8-9w, pool, NORM, CGAP/WM |
| | placenta, neonatal, F |
| | placenta, neonatal, F, NORM, WM |
| | placenta, pool, LICR, EF |
| | prostate stroma, fibroblasts, fetal, 26wM, untreated, AMP |
| | prostate, 28M, NORM |
| | prostate, 32M, SUB, 3′ CGAP |
| | prostate, AH, mw/adenoCA, 48-73M, pool, lg cDNA |
| | prostate, AH, mw/adenoCA, 57M, 5RP |
| | prostate, AH, mw/adenoCA, node mets, 55M, lg/N, m/PROSTUT16 |
| | prostate, MGC, EF |
| | prostate, epithelium, PIN, mw/cancer, M, m/PROETUP02, 3′CGAP |
| | prostate, epithelium, mw/cancer, PIN, 45M, m/PROETUP02, CGAP |
| | skin, dermis, breast, fibroblasts, 31F, untreated |
| | skin, pool, CGAP, EF |
| | skull tumor, chondroid chordoma, 30F |
| | sm airway, epithelial cells, 58M, untreated |
| | sm intestine, fetal, 20wF |
| | sm intestine, fetal, 8-16M/F, pool, NORM |
| | sm intestine, jejunum, 8F |
| | spleen, aw/pancreas neuroendocrine CA, 65F |
| | stomach tumor, adenoCA, poorly differentiated, 3′ CGAP |
| | synovium, rheuA, 75M/56F, pool, NORM |
| | teratoCA line, NT2, t/cytokines, 5C-FL, EF |
| | teratoCA line, hNT2, t/RA, WM/WN |
| | teratoCA line, hNT2, t/mouse leptin, RA |
| | teratoCA line, hNT2, untreated |
| | testis, 16M, NORM |
| | thymus, aw/anencephaly, fetal, 17wF |
| | thymus, aw/congenital heart abnormalities, 2F |
| | thymus, hyperplasia, aw/myasthenia gravis, 16F |
| | tonsil, lymphoid hyperplasia, 6M |
| | ureter tumor, TC CA, 64M, 5RP |
| | urogenital tumor, TC CA, pool, 3′ CGAP |
| | uterus tumor, endometrial adenoCA, F, pooled, 3′ CGAP |
| | uterus tumor, leiomyoma, m/UTRSNON03, UTRSNOT12, UTRSTMR01 |
| | uterus tumor, serous papillary CA, F, pooled, 3′ CGAP |
| | uterus, F, NORM, CGAP/WM/WN |
| | uterus, aw/liver & breast cancer, 46F |
| | uterus, aw/ovarian follicular cysts, 34F |
| | uterus, cervix, cervicitis, mw/leiomyoma, 29F, 5RP |
| | uterus, mw/leiomyomata, 49, 55F, pool, lg cDNA, EF |
| | uterus, myometrium, mw/leiomyoma, 41F, NORM, m/UTRSTUT05 |
| | uterus, myometrium, mw/leiomyoma, 41F, RP, m/UTRSTUT05 |
| | hepatoma (HepG2) |
| | prostate |
| #41509: | brain, aw/hypoplastic left heart, fetal, 23wM, 5RP |
| | brain, dentate nucleus, aw/cholangioCA, 55F, RP |
| | nervous tissue, pool, LICR, EF |
| | ovary, stromal hyperthecosis, mw/dermoid cyst, 45F, RP |
| | brain, astrocytes, fetal, 22wF, untreated |
| | pancreas, 8M, 5RP |
| | Jurkat line, T-cell leukemia, M, t/PMA |
| | Jurkat line, T-cell leukemia, M, t/PMA, Iono-1 hr, 5C-FL, EF |
| | Jurkat line, T-cell leukemia, M, t/PMA-30 min, NORM, EF |
| | aortic smooth muscle line, M |
| | bladder tumor, TC CA, 80F, m/BLADNOT03 |
| | brain tumor, frontal, astrocytoma, 17F |
| | brain tumor, frontal, meningioma, 50M |
| | brain, archaeocortex, hippocampus, aw/cholangioCA, 55F, RP |
| | brain, aw/hypoplastic left heart, fetal, 23wM |
| | brain, aw/muscular atrophy, 3mF, NORM, GEXP |
| | brain, caudate/putamen/nucleus accumbens, aw/CHF, 35M |
| | brain, infant, 10wF, NORM, WM |
| | brain, neocortex, frontal, aw/cholangioCA, 55F, RP |
| | brain, temporal, mw/neuroepithelial tumor, epilepsy, 45M |
| | breast tumor line, T-47D, ductal CA, 54F, 5C-FL, EF |
| | breast tumor, adenoCA, 46F, m/BRSTNOT33, EF |
| | breast, NF breast disease, 32F |
| | breast, PF changes, mw/multifocal ductal CA in situ, 46F |
| | breast, mw/lobular CA, 58F, m/BRSTTUT03 |
| | eye, retina, 9-80M/F, pool, NORM, EF |
| | eye, retina, fetal, pool, WM/WN |
| | heart, right atrium, 51F |
| | kidney epithelial transf embryo line, 293-EBNA, lg cDNA |
| | liver tumor line, C3A, Hepatob, 15M, t/insulin-24 hr, 5C-FL, EF |
| | liver tumor, mets neuroendocrine CA, 62F, m/ LIVRTMR01 |
| | liver, aw/Patau's, anencephaly, fetal, pool, lg cDNA |
| | lung tumor, squamous cell CA, 57M |
| | lung, mw/mets osteoSAR, aw/pleura mets, 58M |
| | lung, right bronchus, asthmatic, 22-51M/F, pool |
| | lymph node, B-lymphocytes, germinal center, pool, MGC, EF |
| | mixed tissue tumor, head/neck, CA, pool, LICR, EF |
| | nasal/cribriform tumor, olfactory neuroblastoma, 45M, 5RP |
| | nervous tissue tumor, CA, pool, LICR, EF |
| | neuroganglion tumor, ganglioneuroma, 9M |
| | ovary, aw/leiomyomata, 36F |
| | ovary, endometriosis, aw/multiple leiomyomata, 47F, 5C-RP |
| | prostate stroma, fibroblasts, fetal, 26wM, untreated |
| | prostate, AH, mw/adenoCA, node mets, 55M, lg/N, m/PROSTUT16 |
| | sm intestine, mw/carcinoid, aw/node mets, 59M, RP |
| | stomach tumor, CA, pool, LICR, EF |
| | synovium, wrist, dorsal, rheuA, 64F |
| | teratoCA line, NT2, t/cytokines, NORM, EF |
| | teratoCA line, hNT2, t/RA + MI |
| | testis, 16M |
| | thyroid, mw/follicular adenoma, 28F |
| | uterus tumor, leiomyoma, m/UTRSNON03, UTRSNOT12, UTRSTMR01 |
| | uterus, endometrium, aw/cystocele, 38F |
| | brain, hippocampus, aw/CHF, 35M, NORM |
| | bronchial epithelium line, NHBE, 54M, Untx, NORM |
| | ganglion, dorsal root, thoracic, aw/lymphoma, 32M, NORM |
| | mesentery tumor, sigmoid, mets mixed-mullerian tumor, 61F |
| | multiple sclerosis, 46M, NORM, WM/WN |
| | sm intestine, duodenum, 16M |
| | Jurkat line, T-cell leukemia, untreated, TIGR |
| | amnion, pool, LICR, EF |
| | bone marrow tumor line, SH-SY5Y, neuroblastoma, F, untreated |
| | bone, rib, aw/Patau's, fetal, 20wM, lg cDNA |
| | brain line, fetal, 17-18wF, RP, 3′ TIGR |
| | brain tumor, frontal, astrocytoma, 47M |
| | brain tumor, frontal, mets hypernephroma, 58M |
| | brain tumor, mixed types, pool, CGAP |
| | brain, aw/hypoplastic left heart, fetal, 23wM, 5RP, EF |
| | brain, aw/hypoplastic left heart, fetal, 23wM, NORM |
| | brain, cerebellum, Huntington's, mw/CVA, 57M |
| | brain, mixed tissues, aw/aortic aneurysm, 45F |
| | brain, temporal, polymicrogyria, gliosis, 5M |
| | breast tumor, ductal CA, pool, CGAP, EF |
| | breast, NF breast disease, 46F |
| | breast, mw/ductal adenoCA, intraductal CA, aw/node mets, 57F |
| | breast, pool, LICR, EF |
| | colon tumor, CA, pool, LICR, EF |
| | colon tumor, adenoCA, NORM, SUB, CGAP |
| | colon, cecum/descending, polyposis, polyp, M/F, pool, SUB |
| | fallopian tube tumor, endometrioid/serous adenoCA, 85F |
| | fetus, 8-9w, pool, NORM, CGAP/WM/WN |
| | fibroblasts, senescent, NORM, WM/WN |
| | ganglion, dorsal root, thoracic, aw/lymphoma, 32M |
| | germ cell tumor, pool, NORM, 3′ CGAP |
| | germ cell tumor, pool, SUB, 3′ CGAP |
| | heart, 56M |
| | heart, coronary artery, endothelial cells, 58M |
| | heart, fetal, 19w, NORM, CGAP/WM/WN |
| | kidney tumor, Wilms', 8mF |
| | kidney tumor, Wilms', pool, WM/WN |
| | kidney, 49M |
| | kidney, pool, NORM, 3′ CGAP |
| | kidney, pool, SUB, 3′ CGAP |
| | liver/spleen, fetal, 20wM, NORM, CGAP/WM/WN |
| | liver/spleen, fetal, 20wM, NORM, WM |
| | lung fibroblast line, HSC172, fetal, untreated, TIGR |
| | lung tumor, neuroendocrine carcinoid, pool, SUB, CGAP |
| | lung tumor, squamous cell CA, pool, 3′, CGAP |
| | lung, fetal, 19w, NORM, CGAP/WM/WN |
| | mammary, epithelial cells, 21F, untreated, NORM |
| | mixed tissues, fetal lung, testis, B-cell, SUB, 3′ CGAP/WN |
| | mixed tissues, includes tumor, M/F, pool, NORM, EF |
| | mixed tissues, includes tumor, SUB, 3′ CGAP |
| | muscle tumor, alveolar rhabdomyoSAR, CGAP |
| | muscle, skeletal, aw/Krabbe, 11mF |
| | muscle, tibial, aw/thrombosis, 41F |
| | ovary tumor, mucinous cystadenoCA, 43F, m/OVARNOT03 |
| | ovary tumor, serous CA, mets colon CA, 44F, 5RP, EF |
| | ovary tumor, serous papillary adenoCA, F, 3′ CGAP |
| | ovary, aw/cardiomyopathy, 59F |
| | pancreas tumor, adenoCA, 3′ CGAP |
| | paraganglion tumor, paraganglioma, aw/renal cell CA, 46M |
| | periph blood, macrophages, adher PBMC, M/F, MLR-24 hr |
| | placenta, neonatal, F |
| | placenta, neonatal, F, NORM, WM |
| | prostate tumor, TC CA, 66M, EF |
| | prostate, 32M, SUB, 3′ CGAP |
| | prostate, AH, mw/adenoCA, 65M |
| | renal vein, smooth muscle cells, 57M, Untx |
| | skin, dermis, breast, fibroblasts, 31F, t/9CIS RA, lg cDNA, EF |
| | skin, foreskin, melanocytes, M, NORM, WM/WN |
| | skull tumor, chondroid chordoma, 30F |
| | sm intestine tumor, ileum, mets endometrial adenoCA, 64F |
| | sm intestine, fetal, 8-16M/F, pool, NORM |
| | sm intestine, fetal, M, RP, EF |
| | spinal cord, base medulla, Huntington's, aw/CVA, 57M |
| | spinal cord, cervical, aw/lymphoma, 32M |
| | stomach tumor, adenoCA, poorly differentiated, 3′ CGAP |
| | testis, aw/cirrhosis, 37M |
| | uterus tumor, endometrial adenoCA, F, pooled, 3′ CGAP |
| | uterus, F, NORM, CGAP/WM/WN |
| | ovary, adenocarcinoma |
| | brain |
| | Brain, hypothalamus |
| | whole embryo, mainly body |
| | normal mesangial cells (NHMC56046-2) |
| #699704: | hepatoma (HepG2) |
| | Bone marrow |
| | Lung, small cell carcinoma |
| | fallopian tube tumor, endometrioid/serous adenoCA, 85F, RP |
| | gallbladder, 25F, TIGR |
| | lung tumor, squamous cell CA, 57M |
| | Jurkat line, T-cell leukemia, M, t/PMA, Iono-1 hr, 5C-FL, EF |
| | aorta, adventitia, 48M |
| | bladder tumor, CA, pool, LICR, EF |
| | brain, substantia nigra, aw/atherosclerosis, CHF, 81F, NORM |
| | brain, temporal cortex, aw/aortic aneurysm, 45F |
| | colon, ascending, CUC, 74M, EF |
| | fetus, 8-9w, pool, NORM, CGAP/WM/WN |
| | ganglion, dorsal root, cervical, aw/lymphoma, 32M |
| | liver, aw/astrocytoma, 32F |
| | lymphocytes, activated Th1 cells, 6-hr AB |
| | muscle, skeletal, mw/malignant hyperthermia, WM/WN |
| | pancreas, type I diabetes, 43F, RP |
| | periph blood, promonocyte line, THP-1, AML, 1M, untreated |
| | prostate tumor line, LNCaP, CA, 50M, untreated, 5RP |
| | testis, 26M, 5C-FL, EF |
| | thyroid, mw/follicular adenoma, 28F |
| | ureter tumor, TC CA, 64M, 5RP |
| | Jurkat line, T-cell leukemia, M, t/PMA |
| | T-lymphocyte tumor, lymphoma, TIGR |
| | T-lymphocytes, allogenic anergic, 40-50M, t/OKT3 3 day |
| | T-lymphocytes, allogenic, 40-50M, untreated |
| | UCB, derived dendritic cells, M, t/PMA, Iono-5 hr |
| | adipose, abdominal, aw/morbid obesity, 68F |
| | adrenal gland, 8M |
| | adrenal gland, aw/pituitary neoplasm, 61F |
| | adrenal glands, aw/anencephaly, fetal, 16wF, lg cDNA |
| | adrenal tumor, adenoma, pool, 3′ CGAP |
| | adrenal tumor, adrenal cortical CA, 49M, 5RP, EF |
| | adrenal tumor, cortical CA, aw/hilar mets, 52M, RP |
| | adrenal tumor, pheochromocytoma, 52F, 5RP |
| | aorta, endothelial cells, 33F, treated |
| | aortic smooth muscle line, M |
| | bladder, mw/TC CA, CA in situ, 60M, m/BLADTUT04 |
| | bladder, mw/TC CA, aw/node mets, 58M, m/BLADTUT03 |
| | bone marrow line, SH-SY5Y, neuroblastoma, 4F, t/6-OHDA, 5RP, EF |
| | bone tumor, Ewing's SAR, CGAP |
| | bone tumor/line, MG-63, osteoSAR/giant cell, M/F, pool, RP, EF |
| | bone, rib, aw/Paget-Schroetter, 57M |
| | brain tumor, frontal, astrocytoma, 47M |
| | brain tumor, glioblastoma, pool, NORM, CGAP |
| | brain tumor, medulloblastoma, NORM, 3′ CGAP |
| | brain, 55M, NORM, WM |
| | brain, allocortex/neocortex, cingulate, aw/CA, 55F, RP |
| | brain, amygdala/entorhinal cortex, aw/CA, 55F, RP, EF |
| | brain, archaeocortex, hippocampus, aw/cholangioCA, 55F, RP |
| | brain, aw/hypoplastic left heart, fetal, 23wM |
| | brain, aw/hypoplastic left heart, fetal, 23wM, NORM |
| | brain, aw/hypoplastic left heart, fetal, 23wM, lg cDNA |
| | brain, aw/muscular atrophy, 3mF, NORM, GEXP |
| | brain, cerebellum, Huntington's, mw/CVA, 57M, RP |
| | brain, cerebellum, TIGR |
| | brain, choroid plexus, Huntington's, mw/CVA, 57M, RP |
| | brain, corpus callosum, AD, 74M |
| | brain, fetal, 23wM, FL-EN, EF |
| | brain, frontal cortex, aw/CHF, 35M |
| | brain, frontal cortex, aw/CHF, 35M, NORM |
| | brain, frontal, gliosis, mw/epilepsy, cerebral palsy, 27M |
| | brain, hippocampus, aw/CHF, 35M |
| | brain, hippocampus, aw/CHF, 35M, NORM |
| | brain, hypothalamus, Huntington's, mw/CVA, 57M, NORM |
| | brain, infant, 10wF, NORM, WM |
| | brain, mixed tissues, aw/CHF, 35M, pool, lg cDNA |
| | brain, mixed tissues, gliosis, 27, 35M, pool, lg cDNA |
| | brain, neurogenic tumor line, SK-N-MC, neuroepithelioma, 14F |
| | brain, parietal cortex, aw/CHF, 35M |
| | brain, pineal gland, aw/AD, 68, 79F, pool, NORM |
| | brain, pineal gland, aw/AD, CHF, DM, 68F |
| | brain, temporal cortex, aw/CHF, 35M |
| | brain, temporal cortex, schizophrenia, aw/COPD, 55M |
| | brain, thalamus, aw/CHF, 35M |
| | brain, thalamus, aw/CHF, 35M, NORM |
| | breast tumor line, T47D, ductal CA, 54F |
| | breast tumor, adenoCA, 45F, m/BRSTNOT09 |
| | breast tumor, adenoCA, 46F, m/BRSTNOT33, EF |
| | breast tumor, adenoCA, 55F, m/BRSTNOT02 |
| | breast tumor, ductal CA, 68F |
| | breast tumor, ductal, F, pool, 3′/5′ CGAP |
| | breast tumor, lobular CA, 59F, m/BRSTNOR01, BRSTNOT16 |
| | breast tumor, low vascular density, control, F |
| | breast, 46F |
| | breast, PF changes, mw/adenoCA, 45F, m/BRSTTUT08 |
| | breast, PF changes, mw/adenoCA, 55F, m/BRSTTUT01 |
| | breast, mw/lobular CA, 58F, m/BRSTTUT03 |
| | breast, mw/lobular CA, 67F |
| | cartilage, knee, chondrocytes, M/F, t/IL-1 |
| | cervical tumor line, HeLa, adenoCA, 31F, t/PMA, CHX 24 hr |
| | cervical tumor line, HeLa, adenoCA, 31F, untreated |
| | colon polyp, aw/adenoCA, tubulovillous adenoma, 40F |
| | colon tumor epithelial line, T84, CA, WM |
| | colon tumor, adenoCA, 3′ CGAP |
| | colon tumor, adenoCA, 3′ , CGAP |
| | colon tumor, adenoCA, 60M, m/COLNNOT07/08/09/11 |
| | colon tumor, adenoCA, pool, 3′ CGAP |
| | colon tumor, adenoCA, pool, NORM, 3′/5′ CGAP |
| | colon tumor, cecum, adenoCA, 70F |
| | colon tumor, cecum, carcinoid, 30F |
| | colon tumor, sigmoid, adenoCA, 62M, m/COLNNOT16 |
| | colon, 25M, WN |
| | colon, aw/Patau's, fetal, 20wM, lg cDNA |
| | colon, aw/gastroparesis, 37F |
| | colon, cecum polyp, aw/adenoCA, 67F |
| | colon, cecum, benign familial polyposis, 16M |
| | colon, mw/adenoCA, aw/node mets, 60M, m/COLNTUT16 |
| | colon, normal/pseudopolyp, Crohn, 16, 26M, pool, lg cDNA |
| | colon, normal/pseudopolyp, Crohn, 16, 26M, pool, lg/N |
| | colon, sigmoid, mw/Crohn's, carcinoid, 40M, m/COLNCRT01 |
| | colon, transverse, Crohn's, 26M |
| | colon, transverse, benign familial polyposis, 16M |
| | coronary artery, smooth muscle cells, 3M, t/TNF, IL-1, SUB |
| | ear, cochlea, fetal, 16-22w, pool, WM |
| | esophagus tumor, adenoCA, 61M |
| | eye, retina, M/F, TIGR |
| | fallopian tube tumor, endometrioid/serous adenoCA, 85F |
| | fallopian tube tumor, endometrioid/serous adenoCA, 85F, 5RP, EF |
| | fibroblasts, senescent, NORM, WM/WN |
| | gallbladder, cholecystitis, cholelithiasis, 18F |
| | gallbladder, cholecystitis, cholelithiasis, 55F, 5RP |
| | ganglion, dorsal root, cervical, aw/lymphoma, 32M, NORM, EF |
| | germ cell tumor, pool, SUB, 3′ CGAP |
| | heart tumor, left atrium, myxoma, 43M |
| | heart, aw/Patau's syndrome, fetal, 20wM, 5RP |
| | heart, fetal, 19w, NORM, CGAP/WM/WN |
| | heart, left atrium, 51F |
| | kidney line, 293-EBNA, t/5AZA, Trichostatin A, 5C-RP |
| | kidney tumor, Wilms', pool, WM/WN |
| | kidney, 49M |
| | kidney, cortex, mw/renal cell CA, 65M |
| | kidney, cortex, mw/renal cell CA, 65M, 5RP |
| | kidney, fetal, 19-23w, M/F, lg cDNA |
| | kidney, fetal, 23wM |
| | kidney, medulla/cortex, mw/renal cell CA, 53F, m/KIDNTUT16 |
| | kidney, mw/renal cell CA, 65M, m/KIDNTUT15 |
| | kidney, pool, SUB, 3′ CGAP |
| | kidney, right/left, aw/Patau's, fetal, 20wM, pool, lg cDNA |
| | liver tumor line, C3A, Hepatob, 15M, Untx |
| | liver tumor line, C3A, Hepatob, 15M, t/APAP-48 hr |
| | liver tumor, mets neuroendocrine CA, 62F, m/ LIVRTMR01 |
| | liver tumor, mets neuroendocrine CA, 72M, 5RP, EF |
| | liver, 29M |
| | liver, 49M, WM |
| | liver, aw/Patau's, anencephaly, fetal, pool, lg cDNA |
| | liver/spleen, fetal, 20wM, NORM, CGAP/WM/WN |
| | liver/spleen, fetal, 20wM, NORM, WM |
| | lung cell line, fetal, 14wM, 3′ CGAP |
| | lung tumor, adenoCA, 63M |
| | lung tumor, neuroendocrine carcinoid, pool, NORM, 3′ CGAP |
| | lung tumor, poorly differentiated, 3′ CGAP |
| | lung tumor, squamous cell CA, 50M |
| | lung tumor, squamous cell CA, 68M |
| | lung tumor, squamous cell CA, pooled, NORM, CGAP |
| | lung, 2M |
| | lung, NORM |
| | lung, asthma, 10M |
| | lung, asthma, 15M |
| | lung, asthma, 17M |
| | lung, aw/Patau's, fetal, 20wM |
| | lung, aw/Patau's, fetal, 20wM, lg cDNA |
| | lung, aw/anencephaly, fetal, 20wF |
| | lung, idiopathic pulmonary disease, SUB |
| | lung, mw/adenoCA, 63M |
| | lung, mw/adenoCA, COPD, 78M |
| | lung, mw/mets osteoSAR, aw/pleura mets, 58M, NORM |
| | lung, pleura, aw/mets leiomyoSAR to lung, 58F, lg cDNA |
| | lymph node, 11F |
| | microvascular, dermal, endothelial cells, 18F, untreated |
| | microvascular, dermal, endothelial cells, neonatal, M |
| | mixed, (melanocytes, uterus, fetal heart), SUB, CGAP/WM/WN |
| | muscle, skeletal |
| | muscle, thigh, ALS, 74F, NORM |
| | neuroganglion tumor, ganglioneuroma, 9M |
| | nose, nasal polyps, pool, lg cDNA |
| | ovary tumor, mets colon adenoCA, 58F |
| | ovary, aw/cardiomyopathy, 59F |
| | ovary, aw/leiomyoma, 47F |
| | ovary, aw/leiomyomata, 36F, NORM |
| | ovary, endometriosis, aw/multiple leiomyomata, 47F, 5C-RP |
| | ovary, stromal hyperthecosis, mw/dermoid cyst, 45F, RP |
| | pancreas tumor, adenoCA, 3′ CGAP |
| | pancreas, islet cells, pool |
| | pancreatic tumor, anaplastic CA, 45F |
| | parathyroid tumor, adenoma, M/F, NORM, WM |
| | parathyroid, hyperplasia, 69F |
| | penis, corpus cavernosum, M |
| | periph blood, dendritic cells, untreated |
| | periph blood, macrophages, adher PBMC, M/F, 72-hr MLR |
| | periph blood, promonocyte line, THP-1, AML, t/PMA |
| | pituitary gland, aw/AD, mets adenoCA, 70F, RP |
| | pituitary gland, aw/schizophrenia, COPD, 55M, NORM |
| | pituitary, 16-70M/F, pool |
| | pituitary, 16-70M/F, pool, RP |
| | placenta, aw/hydrocephalus, fetal, 16w, RP |
| | placenta, aw/hydrocephalus, fetal, 16w/18wM, pool, RP |
| | placenta, fetal, 21wF |
| | placenta, fetal, 8-9w, pool, NORM, CGAP/WM |
| | placenta, fetal, M, WM |
| | placenta, neonatal, F |
| | placenta, neonatal, F, NORM, WM |
| | probable periph blood, eosinophils, asthma, M/F |
| | prostate tumor, adenoCA, 57M, m/PROSNOT06 |
| | prostate tumor, adenoCA, 58M |
| | prostate tumor, adenoCA, 59M, SUB, m/PROSNOT19 |
| | prostate tumor, adenoCA, 59M, m/PROSNOT19 |
| | prostate tumor, adenoCA, 61M |
| | prostate tumor, adenoCA, 65M, m/PROSNOT20 |
| | prostate tumor, adenoCA, 66M, m/PROSNOT15, PROSDIN01 |
| | prostate tumor, adenoCA, M, m/PROSNOT28/PROSTMC01/PROSTMT05 |
| | prostate, 28M, NORM |
| | prostate, 32M, SUB, 3′ CGAP |
| | prostate, AH, mw/adenoCA, 48-73M, pool, lg cDNA |
| | prostate, AH, mw/adenoCA, 55M, m/PROSTUT16 |
| | prostate, AH, mw/adenoCA, 55M, m/PROSTUT16, lg cDNA |
| | prostate, AH, mw/adenoCA, 57M, 5RP |
| | prostate, AH, mw/adenoCA, 57M, m/PROSTUT04 |
| | prostate, AH, mw/adenoCA, 58M |
| | prostate, AH, mw/adenoCA, 65M |
| | prostate, AH, mw/adenoCA, 66M, m/PROSTUT17 |
| | prostate, AH, mw/adenoCA, 67M, m/PROSTUT03 |
| | prostate, AH, mw/adenoCA, 68M |
| | prostate, AH, mw/adenoCA, 73M |
| | prostate, epithelium, PIN, mw/cancer, 45M, m/PROETUP02, CGAP |
| | renal vein, smooth muscle cells, 57M, Untx |
| | skin, leg, erythema nodosum |
| | sm intestine tumor, ileum, mets ovarian adenoCA, 49F |
| | sm intestine, 13M |
| | sm intestine, 31F, RP |
| | sm intestine, aw/Patau's syndrome, fetal, 20wM, 5RP |
| | sm intestine, ileum, 8F |
| | sm intestine, ileum, aw/adenoCA cecum, node mets, 70F, 5RP |
| | sm intestine, ileum, aw/adenoCA cecum, node mets, 70F, RP |
| | sm intestine, ileum, mw/carcinoid, adenoCA, F, pool, lg cDNA |
| | sm intestine, jejunum, 16M |
| | sm intestine, mw/carcinoid, aw/node mets, 59M, RP |
| | spleen tumor, Hodgkin's, 45M |
| | spleen, 8M |
| | spleen, fetal, TIGR |
| | spleen, fetal, pool |
| | stomach tumor, adenoCA, poorly differentiated, 3′ CGAP |
| | stomach, 55M |
| | stomach, gastritis, mw/adenoCA, node mets, 76M, RP |
| | synovium, 75M |
| | synovium, rheuA, 75M/56F, pool, NORM |
| | synovium, wrist, dorsal, rheuA, 64F |
| | teratoCA line, hNT2, t/RA |
| | teratoCA line, hNT2, untreated |
| | teratoCA line, hNT2, untreated, WM/WN |
| | testis, 10-61M, pool, lg cDNA |
| | testis, 16M |
| | testis, 26M |
| | testis, M, NORM, CGAP/WN |
| | thymus, aw/anencephaly, fetal, 17wF |
| | thyroid tumor, follicular CA, CGAP |
| | thyroid tumor, follicular adenoma, 17M |
| | thyroid, TIGR |
| | thyroid, lymphocytic thyroiditis, mw/papillary CA, 13F |
| | thyroid, mw/adenomatous goiter with follicular adenoma, 18F |
| | thyroid, mw/medullary, papillary CA, node mets, 56M |
| | umb cord blood, T-lymphocytes, Th2 cells, untreated |
| | umb cord blood, mononuclear cells, M/F, t/G-CSF, lg cDNA |
| | ureter tumor, TC CA, 69M |
| | urogenital tumor, TC CA, pool, 3′ CGAP |
| | uterus tumor, endometrial adenoCA, F, pooled, 3′ CGAP |
| | uterus tumor, endometrium, adenosquamous CA, 49F, 5RP |
| | uterus tumor, leiomyoma, 37F, 5RP |
| | uterus tumor, leiomyoma, m/UTRSNON03, UTRSNOT12, UTRSTMR01 |
| | uterus, F, NORM, CGAP/WM/WN |
| | uterus, aw/ovarian follicular cysts, 34F |
| | uterus, endometrium, mw/cervical dysplasia, 32F, 5RP |
| | uterus, myometrium, mw/adenoCA, 59F, RP |
| | uterus, myometrium, mw/leiomyoma, 41F, RP, m/UTRSTUT05 |
| | uterus, myometrium, mw/leiomyoma, 41F, m/UTRSTUT05 |
| | uterus, soft tissue, mw/leiomyomata, 33F |
| | kidney |
| | spleen, 2M, 5RP, EF |
| | T-lymphocytes, CD4+, pool, t/anti-CD28 antibodies |
| | eye, retina, 9-80M/F, pool, NORM, EF |
| | lung, 72M |
| | bladder tumor, TC CA, 58M, m/BLADNOT09 |
| | colon, epithelium, 13F |
| | epiglottis, aw/papillary thyroid CA, 71M |
| | kidney, mw/renal cell CA, 43M, m/KIDNTUT14 |
| | lung, fetal, M, RP |
| | mixed tissues, myometrium, smooth muscle cells, 57M/41F, B |
| | osteogenic tumor line, Saos-2, SAR, 11F, untreated |
| | prostate, AH, mw/adenoCA, 66M, m/PROSTUT10 |
| | thymus, 3M |
| | thyroid, lymphocytic thyroiditis, mw/papillary CA, 30F |
| #691401: | cerebellum cortex |
| | brain, cerebellum, aw/COPD, 69M |
| | brain, choroid plexus, Huntington's, mw/CVA, 57M, RP |
| | brain, globus pallidus/substantia innominata, aw/CA, 55F, RP |
| | brain, hippocampus, aw/atherosclerosis, CHF, 81F |
| | brain, pineal gland, aw/AD, 68, 79F, pool, NORM |
| | brain, temporal cortex, aw/aortic aneurysm, 45F |
| | fetus, 8-9w, pool, NORM, CGAP/WM/WN |
| | mixed tissues, fetal lung, testis, B-cell, SUB, 3′ CGAP/WN |
| | ovary tumor, papillary serous CA, F, 3′ CGAP |
| | ovary, stromal hyperthecosis, mw/dermoid cyst, 45F, RP |
| | placenta, aw/hydrocephalus, fetal, 16w, RP |
| | prostate tumor line, LNCaP, CA, 50M, untreated |
| | testis, M, TIGR |
| | testis, aw/cirrhosis, 37M |
| | brain, substantia nigra, aw/atherosclerosis, CHF, 81F, NORM |
| | prostate tumor line, LNCaP, CA, 50M, untreated, 5C-RP |
| | Brain |
| #42916: | Pancreas, adenocarcinoma |
| | human small intestine |
| | testis |
| | liver |
| | bone tumor/line, MG-63, osteoSAR/giant cell, M/F, pool, RP, EF |
| | penis, corpus cavernosum, M |
| | placenta, neonatal, F, NORM, WM |
| | bone tumor line, MG-63, osteoSar, 14M, untreated |
| | colon tumor, hepatic flexure, adenoCA, 55M, m/COLATMT01 |
| | skin, dermis, breast, fibroblasts, 31F, t/9CIS RA, lg cDNA, EF |
| | adrenal glands, aw/anencephaly, fetal, 16wF |
| | bladder, 78F |
| | bladder, mw/TC CA, 80F, m/BLADTUT02 |
| | brain tumor, mixed, pool, MGC, EF |
| | brain, cerebellum, aw/bronchial CA, 64M |
| | breast, NF breast disease, 46F |
| | breast, PF changes, 40F |
| | colon, CUC, 69M |
| | colon, aw/gastroparesis, 37F |
| | colon, mw/adenoCA, aw/node mets, 60M, NORM, m/COLNTUT16 |
| | esophagus, aw/adenoCA, 61M |
| | fat, breast, aw/fibrosis, 38F |
| | iliac artery, endothelial cells, F, t/1% oxygen 24 hr |
| | liver/spleen, fetal, 20wM, NORM, CGAP/WM/WN |
| | lung, right bronchus, nonasthmatic, 18-55M/F, pool |
| | lymph node tumor, axillary, Hodgkin's disease, 45M |
| | lymph node, 16mM |
| | mammary, epithelial cells, 21F, untreated, NORM |
| | microvascular, dermal, endothelial cells, 22F, t/VEGF, EF |
| | ovary, endometriosis, aw/leiomyomata, 39F, NORM, EF |
| | pancreas tumor line, adenoCA/epitheloid CA, pool, MGC, EF |
| | parathyroid tumor, adenoma, M/F, NORM, WM |
| | penis tumor, squamous cell CA, 64M |
| | periph blood, dendritic cells, untreated |
| | periph blood, monocytes, 42F, t/antiIL-10, LPS |
| | placenta, fetal, 21wF |
| | placenta, fetal, M, WM |
| | prostate, AH, mw/adenoCA, 68M |
| | prostate, epithelial cells, 17M, untreated, NORM |
| | skin, foreskin, melanocytes, M, NORM, WM/WN |
| | sm intestine, ileum, mw/carcinoid, 30F |
| | smooth muscle tumor, CA/leiomyoSAR, pool, LICR, EF |
| | testis, 16M |
| | thyroid, aw/CHF, 64F |
| | thyroid, hyperthyroidism, 16F |
| | thyroid, mw/adenomatous goiter with follicular adenoma, 18F |
| | uterus, F, NORM, CGAP/WM/WN |
| | uterus, endometrium, mw/cervical dysplasia, 32F, 5RP |
| | uterus, myometrium, mw/multiple leiomyomata, 45F |
| #33795: | teratocarcinoma (NT2) |
| | whole embryo, mainly head |
| | adrenal tumor, pheochromocytoma, 57F |
| | bladder tumor line, CA/TC CA/papilloma, pool, MGC, EF |
| | brain tumor, frontal, astrocytoma, 47M |
| | brain tumor, frontal, mets hypernephroma, 58M |
| | brain tumor, mixed types, pool, CGAP |
| | brain, aw/muscular atrophy, 3mF, NORM, GEXP |
| | brain, mw/oligoastrocytoma, epilepsy, 26M, NORM |
| | brain, substantia nigra, aw/atherosclerosis, CHF, 81F, NORM |
| | brain, thalamus, aw/CHF, 35M |
| | brain, thalamus, aw/CEF, 35M, NORM |
| | breast lines, MCF7/MCF-10A, adenoCA/fibrocystic, pool, FL-EN, EF |
| | breast tumor, adenoCA, 55F, m/BRSTNOT02 |
| | breast, mw/ductal CA, CA in situ, aw/node mets, 62F |
| | breast, mw/lobular CA, 58F, m/BRSTTUT03 |
| | colon tumor, adenoCA, carcinoid, M/F, pool, NORM |
| | colon tumor, cecum, carcinoid, 30F |
| | colon, aw/anencephaly, fetal, 20wF |
| | germ cell tumor, pool, NORM, 3′ CGAP |
| | germ cell tumor, pool, SUB, 3′ CGAP |
| | heart tumor, left atrium, myxoma, 43M |
| | heart, 44M, NORM |
| | kidney epithelial transf embryo line, 293-EBNA, serum starv |
| | kidney epithelial transf embryo line, 293-EBNA, t/5AZA |
| | kidney epithelial transf embryo line, 293-EBNA, tf/bgal |
| | kidney tumor line, CA/adenoCA/hypernephroma, pool, MGC, EF |
| | kidney, fetal, 19-23w, M/F, lg cDNA |
| | liver tumor, mets neuroendocrine CA, 62F, m/ LIVRTMR01 |
| | lung tumor, neuroendocrine carcinoid, pool, SUB, CGAP |
| | lung, fetal, M, RP |
| | mixed tissues, fetal lung, testis, B-cell, SUB, 3′ CGAP/WN |
| | mixed tissues, includes tumor, pool, SUB, CGAP |
| | multiple sclerosis, 46M, NORM, WM/WN |
| | muscle, skeletal, mw/malignant hyperthermia, WM/WN |
| | olfactory bulb, aw/CA, 39-85M/F, pool, PlyAT, EF |
| | ovary, endometriosis, aw/leiomyomata, 39F |
| | pancreas, islet cells, pool |
| | periph blood, promonocyte line, THP-1, AML, t/5AZA |
| | pituitary gland, aw/AD, mets adenoCA, 70F, RP |
| | pituitary gland, aw/schizophrenia, COPD, 55M, NORM |
| | pituitary gland, pool, CHGC, EF |
| | prostate, 32M, SUB, 3′ CGAP |
| | skin tumor line, melanoma/squamous cell CA, pool, MGC, EF |
| | spleen, 2M |
| | stomach, aw/esophagus adenoCA, 61M, 5RP |
| | stomach, gastritis, mw/adenoCA, node mets, 76M, RP |
| | teratoCA line, hNT2, t/RA + MI, WM/WN |
| | teratoCA line, hNT2, t/mouse leptin, RA |
| | testis, 16M |
| | thymus, hyperplasia, aw/myasthenia gravis, 16F |
| | thyroid, mw/follicular adenoma, 28F |
| | thyroid, mw/medullary, papillary CA, node mets, 56M |
| | brain, globus pallidus/substantia innominata, aw/CA, 55F, RP |
| #728857: | testis |
| | adrenal tumor, pheochromocytoma, 57F |
| | brain, aw/hypoplastic left heart, fetal, 23wM, 5RP |
| | brain, pineal gland, M/F, NORM, WM |
| | kidney, pool, SUB, 3′ CGAP |
| | lung tumor, neuroendocrine carcinoid, pool, SUB, CGAP |
| | lymph node, B-lymphocytes, germinal center, pool, MGC, EF |
| | mixed tissue tumor, head/neck, CA, pool, LICR, EF |
| | testis tumor line, embryonal CA, pool, MGC, EF |
| | fallopian tube tumor, endometrioid/serous adenoCA, 85F, RP |
| | adrenal gland, aw/pituitary neoplasm, 61F |
| | brain, hippocampus, aw/aortic aneurysm, 45F, 5RP |
| | colon, CUC, 69M |
| | coronary artery, smooth muscle cells, 3M, NORM |
| | lymphocytes, activated Th2 cells, 6-hr AB |
| | mixed tissues, includes tumor, SUB, 3′ CGAP |
| | mixed, (melanocytes, uterus, fetal heart), SUB, CGAP/WM/WN |
| | periph blood, dendritic cells, untreated |
| | placenta, neonatal, F, NORM, WM |
| | testis, M, NORM, CGAP/WN |
| | brain, temporal cortex, aw/aortic aneurysm, 45F |
| | colon |
| | Testis |
| #734179: | Skin, melanotic melanoma. |
| | testis |
| | teratocarcinoma (NT2) |
| #36832: | Ovary, adenocarcinoma |
| | brain, archaeocortex, hippocampus, aw/cholangioCA, 55F, RP |
| | kidney, mw/renal cell CA, 8, 53F, pool, NORM |
| | lung, aw/Patau's, fetal, 20wM |
| | mixed tissues, fetal lung, testis, B-cell, SUB, 3′ CGAP/WN |
| | sm intestine, aw/stomach ulcer, 49F, 5RP |
| | spleen, 8M |
| | teratoCA line, hNT2, t/RA + MI, WM/WN |
| | uterus, F, NORM, CGAP/WM/WN |
| | uterus, myometrium, mw/leiomyoma, 41F, RP, m/UTRSTUT05 |
| | breast, PF changes, mw/multifocal ductal CA in situ, 46F |
| | heart, fetal, 19w, NORM, CGAP/WM/WN |
| | ureter tumor, TC CA, 69M |
| | brain, allocortex/neocortex, cingulate, aw/CA, 55F, RP |
| | brain, aw/hypoplastic left heart, fetal, 23wM, 5RP |
| | placenta, neonatal, F, NORM, WM |
| | brain, hippocampus, aw/aortic aneurysm, 45F, 5RP |
| | brain, sensory-motor cortex, aw/CHF, 35M |
| | brain, temporal cortex, aw/aortic aneurysm, 45F |
| | lymph node tumor, mets squamous cell CA, aw/tongue CA, 5RP |
| | sm intestine, 8M |
| | brain, parietal cortex, aw/CHF, 35M |
| | lung, fetal, M, RP |
| | ovary, aw/leiomyomata, 36F, NORM |
| | brain tumor, anaplastic oligodendroglioma, pool, NORM, CGAP |
| | brain, cerebellum, Huntington's, mw/CVA, 57M, RP |
| | colon tumor, hepatic flexure, adenoCA, 55M, SUB, m/COLATMT01, EF |
| | thymus, aw/congenital heart abnormalities, 2F, RP |
| | bone marrow line, SH-SY5Y, neuroblastoma, 4F, t/6-OHDA, 5RP, EF |
| | brain tumor, frontal, astrocytoma, 17F |
| | brain, amygdala/entorhinal cortex, aw/CA, 55F, RP, EF |
| | brain, aw/hypoplastic left heart, fetal, 23wM, 5RP, EF |
| | esophagus tumor, adenoCA, 61M, 5RP, EF |
| | liver, aw/Patau's syndrome, fetal, 20wM, 5RP |
| | lung, pool, LICR, EF |
| | pancreatic tumor, adenoCA, 65F, m/PANCNOT08 |
| | prostate tumor line, LNCaP, CA, 50M, untreated, 5RP |
| | sm intestine, 31F, RP |
| | sm intestine, fetal, M, RP |
| | umbilical artery, endothelial cells, M, untreated, 5RP, EF |
| | fallopian tube tumor, endometrioid/serous adenoCA, 85F, 5RP, EF |
| | brain, hippocampus, AD, 74M |
| | UCB, derived dendritic cells, pool, untreated/treated, NORM |
| | adrenal tumor, adrenal cortical CA, 49M, 5RP, EF |
| | aorta, adventitia, 48M |
| | aortic smooth muscle line, M |
| | bone tumor, sacrum, giant cell tumor, 18F, 5RP, EF |
| | brain tumor, medulloblastoma, NORM, 3′ CGAP |
| | brain, 55M, NORM, WM |
| | brain, aw/hypoplastic left heart, fetal, 23wM |
| | brain, aw/hypoplastic left heart, fetal, 23wM, NORM |
| | brain, cerebellum, TIGR |
| | brain, frontal cortex, aw/CHF, 35M, NORM |
| | brain, frontal, Huntington's, mw/CVA, 57M |
| | brain, frontal, fetal, 5mM, 3′/5′, WN |
| | brain, frontal, gliosis, 46M |
| | brain, frontal, gliosis, mw/epilepsy, cerebral palsy, 27M |
| | brain, frontal, polymicrogyria, gliosis, 5M |
| | brain, hippocampus, 74M, TIGR |
| | brain, hypothalamus, Huntington's, mw/CVA, 57M, NORM |
| | brain, medulla, Huntington's, mw/CVA, 57M |
| | brain, mixed tissues, aw/CHF, 35M, pool, lg/N |
| | brain, neocortex, temporal, aw/cholangioCA, 55F, RP |
| | brain, pineal gland, aw/AD, 68, 79F, pool, NORM |
| | brain, substantia nigra, aw/atherosclerosis, CHF, 81F, NORM |
| | brain, temporal, gliosis, mw/epilepsy, cerebral palsy, 27M |
| | breast, F, NORM, WM |
| | breast, mw/ductal CA, 43F, m/BRSTTUT16 |
| | breast, mw/ductal CA, CA in situ, aw/node mets, 62F |
| | colon tumor, adenoCA, 75M, m/COLNNOT01 |
| | esophagus, mw/adenoCA, aw/node mets, 53M, 5RP |
| | fat, mixed tissues, aw/breast adenoCA, 38-73M/F, pool, NORM |
| | fibroblasts, senescent, NORM, WM/WN |
| | heart, coronary artery, CAD, 46M |
| | heart, coronary artery, endothelial cells, 3M, untreated |
| | lung, pleura, aw/mets leiomyoSAR to lung, 58F, lg cDNA |
| | lymphocytes, nonactivated Th1 cells |
| | ovary tumor, seroanaplastic CA, 52F |
| | penis, corpus cavernosum, M |
| | periph blood, monocytes, 42F, t/anti IL-10, LPS, NORM |
| | pituitary gland, aw/schizophrenia, COPD, 55M, NORM |
| | prostate tumor, TC CA, 66M, EF |
| | prostate, epithelial cells, 17M, untreated, NORM |
| | seminal vesicle, aw/prostate adenoCA, 63M, 5RP |
| | sm intestine, fetal, M, RP, EF |
| | soft tissue tumor, spinal schwannoma, 35M |
| | spinal cord, aw/renal failure, 71M, NORM |
| | synovium, rheuA, 75M/56F, pool, NORM |
| | uterus, endometrium, mw/cervicitis, leiomyoma, pool, lg cDNA |
| | bladder, mw/TC CA, CA in situ, 60M, m/BLADTUT04 |
| | brain tumor, benign meningioma, 35F, NORM |
| | brain tumor, frontal, astrocytoma, 40F, m/BRAINOT14 |
| | brain tumor, frontal, astrocytoma, 47M |
| | brain, acute/chronic multiple sclerosis, pool |
| | brain, cerebellum, AD, 74M |
| | brain, dentate nucleus, aw/cholangioCA, 55F, RP |
| | brain, hippocampus, mw/intracranial hemorrhage, 72F, NORM |
| | brain, mw/oligoastrocytoma, epilepsy, 26M, SUB |
| | brain, pons, aw/CHF, 35M |
| | brain, temporal, polymicrogyria, gliosis, 5M |
| | breast tumor, adenoCA, 54F, m/BRSTNOT03 |
| | breast, papillomatosis, mw/lobular CA, 59F, m/BRSTTUT22 |
| | heart, 44M, NORM |
| | lung, mw/mets osteoSAR, aw/pleura mets, 58M |
| | mixed tissues, includes mw/tumor, 23w-71M/F, pool, 5RP, EF |
| | mixed tissues, includes tumor, SUB, 3′ CGAP |
| | mixed tissues, includes tumor, pool, SUB, CGAP |
| | ovary tumor, papillary serous cystadenoCA 36F, NORM, WM/WN |
| | ovary, stromal hyperthecosis, mw/dermoid cyst, 45F, RP |
| | pituitary, 16-70M/F, pool |
| | prostate tumor, adenoCA, 59M, SUB, m/PROSNOT19 |
| | sm intestine, fetal, 23wM, FL-EN, EF |
| | spleen, aw/hypoplastic left heart, fetal, 23wM, FL-EN, EF |
| | Jurkat line, T-cell leukemia, M, t/PMA, Iono-1 hr, FL-EN, EF |
| | Jurkat line, T-cell leukemia, M, t/PMA-30 min, 5C-FL, EF |
| | T-lymphocyte tumor, lymphoma, TIGR |
| | T-lymphocytes, CD4+, pool, t/anti-CD28 antibodies |
| | T-lymphocytes, allogenic, 40-50M, untreated |
| | UCB, derived dendritic cells, M, t/PMA, Iono-5 hr |
| | adrenal glands, aw/anencephaly, fetal, 16wF, lg cDNA |
| | adrenal tumor, cortical CA, aw/hilar mets, 52M, RP |
| | aorta, endothelial cells, M |
| | bladder tumor, TC CA, 72M |
| | bone marrow tumor line, SH-SY5Y, neuroblastoma, 4F, t/6-OHDA |
| | bone marrow tumor line, SH-SY5Y, neuroblastoma, 4F, t/RA |
| | bone marrow, stem cells, 34+/38+, CGAP |
| | bone, aw/cancer, TIGR |
| | brain line, fetal, 17-18wF, RP, 3′ TIGR |
| | brain tumor, frontal, mets hypernephroma, 58M |
| | brain tumor, frontal, neuronal neoplasm, 32M |
| | brain tumor, frontal, oligoastrocytoma, 50F |
| | brain, astrocytes, fetal, 22wF, untreated |
| | brain, aw/spinal muscular atrophy, 72dF, 3′ SIK/TIGR |
| | brain, caudate/putamen/nucleus accumbens, aw/CHF, 35M |
| | brain, cerebellum, Huntington's, mw/CVA, 57M, 5RP, EF |
| | brain, cerebellum, aw/COPD, 69M |
| | brain, dentate nucleus, aw/CHF, 35M |
| | brain, fetal, 15w, NORM, WM/WN |
| | brain, fetal, 23wM |
| | brain, fetal, 23wM, FL-EN, EF |
| | brain, hippocampus |
| | brain, hippocampus, AD |
| | brain, hippocampus, AD, 77F, EF |
| | brain, hippocampus, aw/CHF, 35M |
| | brain, hippocampus, aw/CHF, 35M, NORM |
| | brain, infant, 10wF, NORM, WM |
| | brain, mixed tissues, aw/CHF, 35M, pool, lg cDNA |
| | brain, mixed tissues, aw/aortic aneurysm, 45F |
| | brain, multiple sclerosis |
| | brain, mw/oligoastrocytoma, epilepsy, 26M, NORM |
| | brain, pineal gland, M/F, NORM, WM |
| | brain, pineal gland, TIGR |
| | brain, pineal gland, aw/AD, CHF, DM, 68F |
| | brain, pineal gland, aw/AD, COPD, 79F |
| | brain, pons, Huntington's, mw/CVA, 57M |
| | brain, striatum/globus pallidus/putamen, aw/CHF, 81F, RP |
| | breast tumor, ductal CA, 68F |
| | breast tumor, ductal CA, F, m/BRSDTMP01, 3′/5′ CGAP |
| | breast tumor, ductal, F, pool, NORM, 3′/5′ CGAP |
| | breast tumor, lobular CA, 58F, m/BRSTNOT05 |
| | breast, 35F |
| | breast, NF breast disease, 46F |
| | breast, PF changes, mw/adenoCA, intraductal CA, 43F |
| | cartilage, OA, M/F |
| | chest wall, soft tissue, mw/adenoCA, aw/COPD, 63M |
| | colon tumor epithelial line, T84, CA, WM |
| | colon tumor, adenoCA, 3′ CGAP |
| | colon tumor, adenoCA, 64F |
| | colon, appendix, aw/leiomyomata, 37F |
| | colon, aw/Patau's, fetal, 20wM, lg cDNA |
| | colon, cecum/descending, polyposis, polyp, M/F, pool, NORM |
| | colon, mw/adenoCA, aw/COPD, 75M, m/COLNTUT02 |
| | eye, retina, 55M, NORM, WM |
| | fallopian tube tumor, endometrioid/serous adenoCA, 85F |
| | fetus, 8-9 w, pool, NORM, CGAP/WM/WN |
| | ganglion, dorsal root, cervical, aw/lymphoma, 32M, NORM, EF |
| | ganglion, dorsal root, thoracic/lumbar, aw/lymphoma, 32M |
| | germ cell tumor, pool, SUB, 3′ CGAP |
| | heart tumor, left atrium, myxoma, 43M |
| | heart, aorta, 12F |
| | hypothalamus, 16-75M/F |
| | kidney epithelial transf embryo line, 293-EBNA, tf/bgal |
| | kidney, pool, SUB, 3′ CGAP |
| | liver tumor line, C3A, Hepatob, 15M, t/MCA-48 hr, SUB |
| | liver, CD34+ progenitor cells, fetal, 20wM |
| | liver, fetal, M, 5RP |
| | lung tumor, adenoCA, 47M |
| | lung tumor, neuroendocrine carcinoid, pool, SUB, CGAP |
| | lung tumor, squamous CA, 50M |
| | lung tumor, squamous cell CA, 50M |
| | lung tumor, squamous cell CA, 57M |
| | lung tumor, squamous cell CA, 64F |
| | lung tumor, squamous cell CA, pooled, NORM, CGAP |
| | lung, 15F |
| | lung, 17F |
| | lung, 2M |
| | lung, asthma, 8F |
| | lung, idiopathic pulmonary disease, SUB |
| | lung, mw/adenoCA, 63M |
| | lung, mw/mets osteoSAR, aw/pleura mets, 58M, NORM |
| | lung, mw/mets thyroid CA, 79M, m/LUNGTUT02 |
| | lung, mw/spindle cell carcinoid, 62F |
| | lymph node, peripancreatic, aw/pancreatic adenoCA, 65M |
| | lymph nodes, 16mM, NORM |
| | mixed tissues, includes tumor, 20w-72M/F, pool, NORM, EF |
| | mixed tissues, includes tumor, 29-69F, pool, NORM, EF |
| | mixed tumor, Wilm's/brain mets, pool, 3′ CGAP |
| | mixed, (melanocytes, uterus, fetal heart), SUB, CGAP/WM/WN |
| | ovary tumor, adenoCA, 58F |
| | ovary tumor, fibrothecoma, F, pool, NORM, 3′, CGAP |
| | ovary tumor, mets colon adenoCA, 58F, NORM |
| | ovary, aw/leiomyomata, 47F |
| | ovary, mw/follicular cysts, 28F |
| | pancreas, 2M |
| | parathyroid, mw/parathyroid CA, 44M |
| | penis tumor, squamous cell CA, 64M |
| | penis, corpora cavernosa, M |
| | periph blood, B-lymphocytes, CLL, pool, NORM, 3′ CGAP |
| | periph blood, dendritic cells, untreated |
| | periph blood, lymphocytes, non-adher PBMC, M/F, t/LPS |
| | periph blood, macrophages, adher PBMC, M/F, MLR-24 hr |
| | periph blood, promonocyte line, THP-1, AML, t/5AZA |
| | pituitary gland, aw/AD, mets adenoCA, 70F, RP |
| | placenta, aw/hydrocephalus, fetal demise, 16w, 18wM, pool |
| | placenta, aw/hydrocephalus, fetal, 16w, FL-EN, EF |
| | prostate tumor line, invasive, M, untreated, 3′ CGAP |
| | prostate tumor, adenoCA, 50M, m/PROSNOT02 |
| | prostate tumor, adenoCA, 60M, m/PROSNOT14 |
| | prostate tumor, adenoCA, 66M, m/PROSNOT15, PROSDIN01 |
| | prostate tumor, adenoCA, 68M, m/PROSTMT03 |
| | prostate tumor, cancer, 45M, m/PROETMP01/02, CGAP |
| | prostate, 21M, TIGR |
| | prostate, AH, mw/adenoCA, 60M, m/PROSTUT08 |
| | prostate, AH, mw/adenoCA, 66M |
| | prostate, AH, mw/adenoCA, 73M, FL-EN, EF |
| | prostate, AH, mw/adenoCA, M, m/PROSTUT13, PROSTUS08/19/20/25 |
| | prostate, epithelium, mw/tumor, M, m/PROSTUP02, 3′ CGAP |
| | renal vein, smooth muscle cells, 57M t/TNF, IL1 |
| | seminal vesicle, aw/adenoCA, 61M |
| | skin, breast and fetal, aw/intraductal CA, pool |
| | skin, breast, aw/adenoCA, 70F |
| | skin, epidermal breast keratinocyte line, NHEK, 30F, Untx |
| | skin, leg, erythema nodosum |
| | skin, leg, keratinocytes, neonatal, M |
| | sm airway, epithelial cells, 58M, untreated |
| | sm intestine tumor, ileum, mets endometrial adenoCA, 64F |
| | sm intestine, duodenum, aw/pancreatic cystadenoma, 41F |
| | sm intestine, ileum, aw/adenoCA cecum, node mets, 70F |
| | sm intestine, ileum, aw/adenoCA cecum, node mets, 70F, 5RP |
| | sm intestine, jejunum, 16M |
| | spinal cord, aw/renal failure, 71M |
| | spleen, Gaucher's, 22M |
| | spleen, fetal, 23wM |
| | spleen, splenomegaly, 59M, EF |
| | synovium, knee, OA, 82F |
| | synovium, wrist, rheuA, 56F |
| | teratoCA line, hNT2, t/RA + MI |
| | testis, 26M |
| | thymus, aw/parathyroid adenoma, 21M |
| | thymus, aw/patent ductus arteriosus, 3M |
| | thymus, hyperplasia, aw/myasthenia gravis, 16F |
| | thyroid, lymphocytic thyroiditis, mw/papillary CA, 30F |
| | tibia, periosteum, mw/osteoSAR, osteogenesis imperfecta, 20M |
| | tonsil, B-lymphocytes, germinal, aw/tonsillitis, NORM, CGAP |
| | tonsil, lymphoid hyperplasia, 6M/9F, pool, AMP |
| | umb cord blood, T-lymphocytes, Th2 cells, untreated |
| | umb cord blood, mononuclear cells, M/F, t/G-CSF, lg cDNA |
| | umbilical vein, endothelial cells, HUVEC, t/TNFa-48 hr, 5C-FL, EF |
| | uterus tumor, leiomyoma, m/UTRSNON03, UTRSNOT12, UTRSTMR01 |
| | uterus, cervix, 40F |
| | uterus, endometrium, mw/cervical dysplasia, 32F, FL-EN, EF |
| | uterus, endometrium, mw/endometrial polyp, 35F |
| | uterus, endometrium, type II defect, endometriosis, F |
| | adrenal gland, 8M |
| | breast, mw/lobular CA, 58F, m/BRSTTUT03 |
| | kidney tumor, clear cell type cancer, pool, SUB, CGAP |
| | liver tumor line, C3A, Hepatob, 15M, t/PB-48 hr |
| | muscle, arm, ALS, 74F |
| | periph blood, monocytes, 42F, t/antiIL-10, LPS |
| | periph blood, promonocyte line, THP-1, AML, 1M, t/tuberculosis |
| | placenta, fetal, 18wM |
| | thyroid, AH, mw/papillary CA, 22F, 5RP, EF |
| | uterus tumor, endometrial adenoCA, F, pooled, 3′ CGAP |
| | teratocarcinoma (NT2) |
| | brain |
| | amygdala |
| #50258: | brain tumor, posterior fossa, meningioma, 70M |
| | breast tumor, lobular CA in situ, F, CGAP |
| | breast, mw/ductal adenoCA, 62F, RP |
| | esophagus tumor, adenoCA, 61M, 5RP, EF |
| | fat, abdomen, aw/obesity, 52F |
| | prostate, AH, mw/adenoCA, node mets, 55M, lg/N, m/PROSTUT16 |
| | seminal vesicle, aw/prostate adenoCA, 63M, 5RP |
| | sm intestine, ileum, chronic inflammation, 29F, 5RP, EF |
| | spleen, ITP, 8, 14M, pool, lg cDNA |
| | UCB, derived dendritic cells, pool, untreated/treated, NORM |
| | adenoid, inflamed, 3 |
| | adrenal gland, aw/renal cell CA, 43M |
| | bladder tumor, TC CA, 60M, m/BLADNOT05 |
| | brain stem, aw/DMt1, 72M, NORM |
| | brain tumor, anaplastic oligodendroglioma, pool, NORM, CGAP |
| | brain tumor, benign meningioma, 35F, 5RP |
| | breast, NF breast disease, 35F |
| | breast, PF changes, mw/adenoCA, 55F, m/BRSTTUT01 |
| | breast, mw/ductal adenoCA, intraductal CA, aw/node mets, 57F |
| | clavicle, osteoblasts, 40M, untreated, lg cDNA, EF |
| | colon tumor, adenoCA, 64F |
| | colon tumor, adenoCA, pool, NORM, 3′ CGAP |
| | colon, Crohn's, mw/benign carcinoid, 40M, m/COLNNOT05 |
| | colon, aw/Patau's, fetal, 20wM, lg cDNA |
| | colon, descending, CUC, 28M, 5RP |
| | colon, descending, benign familial polyposis, 16M |
| | colon, mw/adenoCA, aw/COPD, 75M, m/COLNTUT02 |
| | fetus, 8-9w, pool, NORM, CGAP/WM/WN |
| | gallbladder, cholecystitis, cholelithiasis, 18F |
| | ganglia, sympathetic trunk, 16M, CGAP, EF |
| | heart, aorta, 17F |
| | heart, fetal, 19w, NORM, CGAP/WM/WN |
| | kidney tumor, clear cell type cancer, pool, SUB, CGAP |
| | kidney, medulla/cortex, mw/renal cell CA, 53F, m/KIDNTUT16 |
| | kidney, pool, NORM, 3′ CGAP |
| | kidney, pool, SUB, 3′ CGAP |
| | liver tumor line, C3A, Hepatob, 15M, t/MCA-48 hr |
| | liver/spleen, fetal, 20wM, NORM, CGAP/WM/WN |
| | lung tumor, adenoCA, 47M |
| | lung tumor, neuroendocrine carcinoid, pool, SUB, CGAP |
| | lung, aw/anencephaly, fetal, 20wF |
| | lung, mw/mets osteoSAR, aw/pleura mets, 58M |
| | lung, mw/mets osteoSAR, aw/pleura mets, 58M, NORM |
| | lung, mw/spindle cell carcinoid, 62F |
| | lung, pleura, aw/mets leiomyoSAR to lung, 58F, lg cDNA |
| | lymph node tumor, follicular lymphoma, 3′ CGAP |
| | lymph node, 11F |
| | lymphocytes, nonactivated Th1 cells |
| | microvascular, dermal, endothelial cells, 18F, untreated |
| | mixed tissues, fetal lung, testis, B-cell, SUB, 3′ CGAP/WN |
| | mixed tissues, includes tumor, M/F, pool, NORM, EF |
| | mixed tissues, includes tumor, SUB, 3′ CGAP |
| | multiple sclerosis, 46M, NORM, WM/WN |
| | ovary tumor, mucinous cystadenoCA, 43F, m/OVARNOT03 |
| | ovary, endometriosis, 24F |
| | periph blood, eosinophils, asthma, M/F |
| | periph blood, macrophages, adher PBMC, M/F, 48-hr MLR |
| | periph blood, monocytes, 42F, t/antiIL-10, LPS |
| | periph blood, promonocyte line, THP-1, AML, t/5AZA, SUB |
| | placenta, aw/hydrocephalus, fetal demise, 16w, 18wM, pool |
| | prostate tumor, adenoCA, 59M, SUB, m/PROSNOT19 |
| | prostate, 32M, SUB, 3′ CGAP |
| | prostate, AH, mw/adenoCA, 66M, m/PROSTUT17 |
| | prostate, mw/adenoCA, 65M |
| | prostate, mw/adenoCA, 68M, m/PROSTUT18 |
| | sm intestine, 31F, RP |
| | sm intestine, ileum, aw/adenoCA cecum, node mets, 70F, RP |
| | sm intestine, mw/carcinoid, aw/node mets, 59M |
| | spinal cord, base medulla, Huntington's, 57M, lg cDNA, EF |
| | spleen, 2M |
| | synovium, 75M |
| | testis tumor, seminoma, 45M |
| | thymus, 3M |
| | thymus, hyperplasia, aw/myasthenia gravis, 16F |
| | thyroid, lymphocytic thyroiditis, mw/papillary CA, 13F |
| | umb cord blood, T-lymphocytes, Th2 cells, untreated |
| | uterus tumor, endometrial adenoCA, F, pooled, 3′ CGAP |
| | uterus, endometrium, mw/leiomyoma aw/endometriosis, 48F |
| | kidney |
| | breast, papillomatosis, mw/lobular CA, 59F, m/BRSTTUT22, RP |
| | cartilage, OA, M/F |
| | colon tumor, cecum, adenoCA, 70F |
| | fallopian tube tumor, endometrioid/serous adenoCA, 85F, 5RP, EF |
| | kidney epithelial transf embryo line, 293-EBNA, tf/bgal |
| | lung tumor, squamous cell CA, 65F |
| | lymph node tumor, axillary, Hodgkin's disease, 45M |
| | penis, corpus cavernosum, M |
| | sm intestine, 15F |
| | sm intestine, mw/carcinoid, aw/node mets, 59M, RP |
| | thymus, aw/congenital heart abnormalities, 2F, RP |
| | thymus, aw/patent ductus arteriosus, 3M, 5RP |
| | umb cord blood, mononuclear cells, M/F, t/G-CSF |
| | brain, aw/hypoplastic left heart, fetal, 23wM, 5RP, EF |
| | cervical tumor line, HeLa, adenoCA, 31F, t/PMA, CHX 4 hr |
| | prostate, epithelial cells, 17M, untreated |
| | synovium, rheuA, 75M/56F, pool, NORM |
| | thyroid, AH, mw/papillary CA, 22F, 5RP, EF |
| | placenta, aw/hydrocephalus, fetal, 16w, 5RP |
| | sm intestine, aw/Patau's syndrome, fetal, 20wM, 5RP |
| | uterus tumor, leiomyoma, 34F |
| | uterus, endometrium, type I defect, 28F |
| | uterus, myometrium, mw/leiomyoma, 41F, RP, m/UTRSTUT05 |
| #720486: | umb cord blood, mononuclear cells, t/IL-5 |
| | kidney, pool, SUB, 3′ CGAP |
| | lung, NORM |
| | lung, idiopathic pulmonary disease, NORM |
| | lymph node, 16mM |
| | mixed, (melanocytes, uterus, fetal heart), SUB, CGAP/WM/WN |
| | thyroid, lymphocytic thyroiditis, mw/papillary CA, 13F |
| #51386: | brain |
| | brain, mixed tissues, Nrml/meningioma/AD, pool, lg cDNA, EF |
| | lymph node, B-lymphocytes, germinal center, pool, MGC, EF |
| | mixed tissues, includes tumor, pool, SUB, CGAP |
| | placenta, pool, LICR, EF |
| | germ cell tumor, pool, NORM, 3′ CGAP |
| | tonsil, B-lymphocytes, germinal, aw/tonsillitis, NORM, CGAP |
| | brain, aw/hypoplastic left heart, fetal, 23wM, 5RP, EF |
| | brain, mixed tissues, aw/aortic aneurysm, 45F |
| | breast, mw/ductal CA, CA in situ, aw/node mets, 62F |
| | fetus, 7-8w, pool, AMP, EF |
| | heart, 65M |
| | kidney, cortex, mw/renal cell CA, 65M |
| | synovium, wrist, rheuA, 62F |
| | brain, frontal cortex, aw/CHF, 35M, NORM |
| | colon, pool, LICR, EF |
| | periph blood, B-lymphocytes, CLL, pool, NORM, 3′ CGAP |
| | pituitary gland, aw/AD, mets adenoCA, 70F, RP |
| | brain, temporal cortex, aw/aortic aneurysm, 45F, RP |
| | gallbladder, cholecystitis, cholelithiasis, 53F |
| | probable astrocytes, M/F, untreated |
| | Jurkat line, Frac Nuc, T-cell leukemia, M, t/antiCD3, 5C-FL, EF |
| | Jurkat line, T-cell leukemia, M, t/PMA |
| | T-lymphocytes, CD4+, pool, t/anti-CD3 antibodies |
| | adenoid, inflamed, 3 |
| | bladder, mw/TC CA, 80F, m/BLADTUT02 |
| | bone marrow tumor, CA, pool, LICR, EF |
| | bone tumor line, osteoSAR, MGC, EF |
| | brain tumor, frontal, astrocytoma, 17F |
| | brain tumor, frontal, astrocytoma, 47M |
| | brain tumor, frontal, oligoastrocytoma, 50F |
| | brain, archaeocortex, hippocampus, aw/cholangioCA, 55F, RP |
| | brain, fetal, 23wM |
| | brain, frontal cortex, schizophrenic, 34M, RP, WM/WN |
| | brain, parietal cortex, aw/CHF, 35M |
| | brain, sensory-motor cortex, aw/CHF, 35M |
| | bronchial epithelium line, NHBE, 54M, Untx |
| | colon line, pool, LICR, EF |
| | colon tumor, adenoCA, NORM, SUB, CGAP |
| | colon tumor, sigmoid, adenoCA, 62M, m/COLNNOT16 |
| | colon, aw/gastroparesis, 37F |
| | colon, normal/pseudopolyp, Crohn, 16, 26M, pool, lg/N |
| | germ cell tumor, pool, SUB, 3′ CGAP |
| | kidney, aw/anencephaly, fetal, 17wF |
| | kidney, fetal, 23wM |
| | liver tumor line, C3A, Hepatob, 15M, Untx, NORM |
| | liver, mw/mets neuroendocrine CA, 62F, RP, m/LIVRTUT13, EF |
| | liver/spleen, fetal, 20wM, NORM, WM |
| | lung tumor, adenoCA, 70F |
| | lung, 12M |
| | lung, 72M, WM/WN |
| | lung, aw/Patau's, fetal, 20wM |
| | lung, fetal, M, RP |
| | lung, mw/spindle cell carcinoid, 62F |
| | lymph node tumor, follicular lymphoma, 3′ CGAP |
| | mammary, epithelial cells, 21F, untreated, NORM |
| | mixed tumor, Wilm's/brain mets, pool, 3′ CGAP |
| | muscle tumor line, rhabdomyosarcoma, MGC, EF |
| | muscle, arm, ALS, 74F |
| | nervous tissue, pool, LICR, EF |
| | osteogenic tumor line, Saos-2, SAR, 11F, untreated |
| | ovary, endometriosis, aw/leiomyomata, 39F, NORM, EF |
| | pancreas tumor, adenoCA, 3′ CGAP |
| | periph blood, eosinophils, hypereosinophilia, 48M |
| | periph blood, monocytes, 42F, t/IL-10, LPS, NORM |
| | periph blood, promonocyte line, THP-1, AML, 1M, t/tuberculosis |
| | periph blood, promonocyte line, THP-1, AML, t/5AZA, SUB |
| | periph blood, promonocyte line, THP-1, AML, untreated |
| | peritoneum tumor, neuroendocrine CA, 66F |
| | prostate, AH, mw/adenoCA, 57M, 5RP |
| | prostate, AH, mw/adenoCA, 58M |
| | sm intestine, ileum, mw/carcinoid, 30F |
| | synovium, knee, OA, 82F |
| | testis, 10-61M, pool, lg cDNA |
| | thymus, aw/patent ductus arteriosus, 3M, 5RP |
| | thymus, fetal, pool, NORM, CGAP |
| | umb cord blood, T-lymphocytes, Th2 cells, untreated |
| | uterus tumor, endometrium, adenosquamous CA, 49F, 5RP |
| | uterus tumor, serous papillary CA, F, pooled, 3′ CGAP |
| | uterus, aw/ovarian follicular cysts, 34F |
| | white blood cells, 27F |
| | fetus, 8-9w, pool, NORM, CGAP/WM/WN |
| #35501: | UCB, derived dendritic cells, pool, untreated/treated, NORM |
| | colon tumor, adenoCA, 3′, CGAP |
| | lymphocytes, nonactivated Th1 cells |
| | microvascular, dermal, endothelial cells, neonatal, M |
| | stomach, aw/esophagus adenoCA, 61M, 5RP |
| | skin, dermis, breast, fibroblasts, 31F, t/9CIS RA, lg cDNA, EF |
| | CML precursor line, K-562, 53F, t/PMA-96 hr |
| | Jurkat line, T-cell leukemia, M, t/PMA |
| | Jurkat line, T-cell leukemia, M, untreated |
| | Jurkat line, T-cell leukemia, untreated, TIGR |
| | PBMC, 60M, untreated |
| | T-B lymphoblast line, leukemia, untreated |
| | T-lymphocyte tumor, lymphoma, TIGR |
| | T-lymphocytes, CD4+, pool, t/anti-CD28 antibodies |
| | T-lymphocytes, CD4+, pool, t/anti-CD3 antibodies |
| | T-lymphocytes, activated, TIGR |
| | T-lymphocytes, allogenic anergic, 40-50M, t/OKT3 3 day |
| | UCB, derived dendritic cells, M |
| | adenoid, inflamed, 3 |
| | adrenal gland, 20M |
| | adrenal glands, aw/anencephaly, fetal, 16wF, lg cDNA |
| | adrenal tumor, adenoma, pool, 3′ CGAP |
| | adrenal tumor, pheochromocytoma, 43F, m/ADRENOT11 |
| | adrenal tumor, pheochromocytoma, 52F, EF |
| | aorta, adventitia, 65F |
| | aorta, endothelial cells, M |
| | bladder and seminal vesicle, 28M |
| | bladder tumor, TC CA, 72M |
| | bladder tumor, TC CA, 80F, m/BLADNOT03 |
| | bladder, 11M |
| | bladder, 11M, RP |
| | bladder, 78F |
| | bladder, chronic cystitis, aw/urethral adenoCA, 73M |
| | bladder, mw/TC CA, CA in situ, 60M, m/BLADTUT04 |
| | bladder, mw/TC CA, aw/prostate TC CA, 66M, m/BLADTUT05 |
| | bone marrow line, SH-SY5Y, neuroblastoma, 4F, t/6-OHDA, 5RP, EF |
| | bone marrow tumor line, SH-SY5Y, neuroblastoma, 4F, t/RA |
| | bone marrow tumor line, SH-SY5Y, neuroblastoma, F, untreated |
| | bone marrow, 16-70M/F |
| | bone marrow, CD34+ progenitor cells, M, AMP |
| | bone tumor line, MG-63, osteoSar, 14M, untreated |
| | bone tumor, Ewing's SAR, CGAP |
| | bone tumor, sacrum, giant cell tumor, 18F |
| | brain line, fetal, 17-18wF, RP, 3′ TIGR |
| | brain stem, aw/DMt1, 72M, NORM |
| | brain tumor, anaplastic oligodendroglioma, pool, NORM, CGAP |
| | brain tumor, frontal, astrocytoma, 17F |
| | brain tumor, frontal, astrocytoma, 40F, m/BRAINOT14 |
| | brain tumor, frontal, astrocytoma, 47M |
| | brain tumor, frontal, meningioma, 61F |
| | brain tumor, frontal, mets hypernephroma, 58M |
| | brain tumor, frontal, oligoastrocytoma, 50F |
| | brain tumor, glioblastoma, pool, NORM, CGAP |
| | brain tumor, meningioma, pool, 3′, CGAP |
| | brain tumor, mixed types, pool, CGAP |
| | brain, acute/chronic multiple sclerosis, pool |
| | brain, amygdala, aw/CHF, 35M |
| | brain, amygdala/entorhinal cortex, aw/CA, 55F, RP, EF |
| | brain, archaeocortex, hippocampus, aw/cholangioCA, 55F, RP |
| | brain, aw/hypoplastic left heart, fetal, 23wM |
| | brain, aw/hypoplastic left heart, fetal, 23wM, NORM |
| | brain, aw/muscular atrophy, 3mF, NORM, GEXP |
| | brain, aw/spinal muscular atrophy, 72dF, NORM, SIK |
| | brain, caudate nucleus, schizophrenia, 66F |
| | brain, caudate/putamen/nucleus accumbens, aw/CHF, 35M |
| | brain, cerebellum, AD, 74M |
| | brain, cerebellum, Huntington's, mw/CVA, 57M |
| | brain, cerebellum, Huntington's, mw/CVA, 57M, RP |
| | brain, cerebellum, vermis, AD, 79-87M/F, pool, AMP/N |
| | brain, cingulate, aw/MI, 85F, AMP/N |
| | brain, corpus callosum, AD, 74M |
| | brain, fetal, 15w, NORM, WM/WN |
| | brain, frontal cortex, schizophrenic, 34M, RP, WM/WN |
| | brain, frontal, Huntington's, mw/CVA, 57M |
| | brain, frontal, fetal, 5mM, 3′/5′, WN |
| | brain, frontal, gliosis, mw/epilepsy, cerebral palsy, 27M |
| | brain, frontal, mw/astrocytoma, 40F, m/BRAITUT12/ BRAFTUE03 |
| | brain, frontal, polymicrogyria, gliosis, 5M |
| | brain, globus pallidus/substantia innominata, aw/CHF, 35M |
| | brain, hippocampus, AD, 74M |
| | brain, hippocampus, AD, 77F, EF |
| | brain, hippocampus, aw/CHF, 35M |
| | brain, hippocampus, mw/intracranial hemorrhage, 72F, NORM |
| | brain, infant, 10wF, NORM, WM |
| | brain, infant, F, TIGR |
| | brain, midbrain, aw/CHF, 35M |
| | brain, mixed tissues, aw/aortic aneurysm, 45F |
| | brain, mixed tissues, aw/cholangioCA, 55F, RP |
| | brain, mixed, archaecortex/hippocampus, 55F, pool, AMP/N |
| | brain, multiple sclerosis |
| | brain, mw/oligoastrocytoma, epilepsy, 26M, NORM |
| | brain, mw/oligoastrocytoma, epilepsy, 26M, SUB |
| | brain, neocortex, temporal, aw/cholangioCA, 55F, RP |
| | brain, occipital, Huntington's, mw/CVA, 57M |
| | brain, parietal cortex, aw/CHF, 35M |
| | brain, parietal cortex, aw/CHF, 35M, AMP/N |
| | brain, pineal gland, TIGR |
| | brain, pineal gland, aw/AD, 68, 79F, pool, NORM |
| | brain, sensory-motor cortex, aw/CHF, 35M |
| | brain, striatum, caudate nucleus, schizophrenia, 49M |
| | brain, substantia nigra, aw/atherosclerosis, CHF, 81F, NORM |
| | brain, temporal cortex, aw/CHF, 35M |
| | brain, temporal cortex, aw/aortic aneurysm, 45F |
| | brain, temporal, gliosis, mw/epilepsy, 27M, lg cDNA |
| | brain, temporal, gliosis, mw/epilepsy, cerebral palsy, 27M |
| | brain, temporal, mw/neuroepithelial tumor, epilepsy, 45M |
| | brain, thalamus, aw/CHF, 35M |
| | brain, thalamus, aw/CHF, 35M, NORM |
| | breast tumor line, Hs 578T, ductal CA, 74F, t/EGF-8 hr, 5C-FL, EF |
| | breast tumor, adenoCA, 45F, m/BRSTNOT09 |
| | breast tumor, adenoCA, 46F, m/BRSTNOT17 |
| | breast tumor, adenoCA, 55F, m/BRSTNOT02 |
| | breast tumor, ductal CA, 43F, m/BRSTTMT01 |
| | breast tumor, ductal adenoCA, 66F |
| | breast tumor, lobular CA, 58F, m/BRSTNOT05 |
| | breast, 46, 60F, pool, NORM |
| | breast, 56F |
| | breast, NF breast disease, 32F |
| | breast, NF breast disease, 46F |
| | breast, NF changes, mw/ductal adenoCA, 40-57F, pool, lg cDNA |
| | breast, PF changes, mw/adenoCA, 55F, m/BRSTTUT01 |
| | breast, PF changes, mw/adenoCA, intraductal CA, 43F |
| | breast, PF changes, mw/ductal adenoCA, 54F, m/BRSTTUT02 |
| | breast, PF changes, mw/intraductal cancer, 48F |
| | breast, mw/ductal CA, CA in situ, aw/node mets, 62F |
| | breast, mw/ductal adenoCA, 46F, m/BRSTTUS08, BRSTTUT13 |
| | breast, mw/ductal adenoCA, aw/node mets, 46F, m/BRSTTUT15 |
| | breast, mw/lobular CA, 67F |
| | bronchial epithelium line, NHBE, 54M, Untx |
| | cartilage, OA |
| | cervical tumor line, HeLa S3, adenoCA, 31F, untreated, WM/WN |
| | cervical tumor line, HeLa, adenoCA, 31F, t/PMA, CHX 24 hr |
| | colon tumor, adenoCA, 64F |
| | colon tumor, adenoCA, pool, 3′ CGAP |
| | colon tumor, adenoCA, pool, NORM, 3′ CGAP |
| | colon tumor, adenoCA, pool, NORM, 3′/5′ CGAP |
| | colon tumor, cecum, adenoCA, 45F |
| | colon tumor, cecum, adenoCA, 70F |
| | colon tumor, cecum, carcinoid, 30F |
| | colon tumor, juvenile granulosa cell, 3′ CGAP |
| | colon tumor, sigmoid, adenoCA, 62M, m/COLNNOT16 |
| | colon tumor, villous adenoma, 3′ CGAP |
| | colon, TIGR |
| | colon, aw/Patau's, fetal, 20wM, lg cDNA |
| | colon, aw/anencephaly, fetal, 20wF |
| | colon, epithelium, 13F, RP |
| | colon, mw/adenoCA, aw/node mets, 60M, m/COLNTUT16 |
| | colon, normal/pseudopolyp, Crohn, 16, 26M, pool, lg cDNA |
| | colon, normal/pseudopolyp, Crohn, 16, 26M, pool, lg/N |
| | colon, sigmoid, mw/Crohn's, carcinoid, 40M, m/COLNCRT01 |
| | colon, sigmoid, mw/adenoCA, aw/node mets, 62M, m/COLNTUT03 |
| | colon, transverse, benign familial polyposis, 16M |
| | colon, ulcerative colitis, 16M |
| | coronary artery, smooth muscle cells, 3M, NORM |
| | esophagus tumor, adenoCA, 61M, 5RP, EF |
| | esophagus tumor, squamous cell CA, 3′, CGAP |
| | esophagus, mw/adenoCA, aw/node mets, 53M, 5RP |
| | eye, retina, 55M, NORM, WM |
| | eye, retina, 9-80M/F, pool, NORM, EF |
| | fat, mesentery, aw/diverticulosis, diverticulitis, 71M |
| | fetus, 8-9w, pool, NORM, CGAP/WM/WN |
| | ganglion, dorsal root, cervical, aw/lymphoma, 32M |
| | ganglion, dorsal root, thoracic, aw/lymphoma, 32M |
| | germ cell tumor, pool, NORM, 3′ CGAP |
| | germ cell tumor, pool, SUB, 3′ CGAP |
| | germ cell tumor, yolk sac, 3′ CGAP |
| | heart coronary artery endothelial cells, 3M, untreated, NORM |
| | heart tumor, left atrium, myxoma, 43M |
| | heart, 44M |
| | heart, 44M, NORM |
| | heart, coronary artery, endothelial cells, 3M, untreated |
| | heart, fetal, 19w, NORM, CGAP/WM/WN |
| | heart, fetal, 8-10w, pool, BI |
| | heart, hypoplastic left, fetal; 23wM |
| | heart, left atrium, 51F |
| | heart, left ventricle, 51F |
| | heart, left ventricle, Pompe's, 7mM |
| | heart, left ventricle, mw/myocardial infarction, 56M |
| | heart, right atrium, 39M |
| | kidney epithelial transf embryo line, 293-EBNA, Untx |
| | kidney epithelial transf embryo line, 293-EBNA, serum starv |
| | kidney epithelial transf embryo line, 293-EBNA, t/5AZA |
| | kidney epithelial transf embryo line, 293-EBNA, tf/E2F1, DP1 |
| | kidney epithelial transf embryo line, 293-EBNA, tf/bgal |
| | kidney tumor, Wilms', pool, WM/WN |
| | kidney tumor, renal cell CA, 43M, m/KIDNNOT20 |
| | kidney tumor, renal cell CA, pool, 3′/5′ CGAP |
| | kidney, aw/hypoplastic left heart, fetal, 23wM, 5RP |
| | kidney, fetal, 19-23w, M/F, lg cDNA |
| | kidney, mw/renal cell CA, 65M, m/KIDNTUT15 |
| | kidney, mw/renal cell CA, 8, 53F, pool, NORM |
| | kidney, pool, NORM, 3′ CGAP |
| | kidney, pool, SUB, 3′ CGAP |
| | liver tumor, hepatoma, 50M |
| | liver tumor, mets colon adenoCA, 51F |
| | liver tumor, mets neuroendocrine CA, 62F, m/ LIVRTMR01 |
| | liver tumor, mets neuroendocrine CA, 72M, 5RP, EF |
| | liver, aw/Patau's, anencephaly, fetal, pool, lg cDNA |
| | liver, hepatocytes, mw/CA, M, m/LIVHTUP01, CGAP |
| | liver, mw/mets neuroendocrine CA, 62F, RP, m/LIVRTUT13, EF |
| | liver, pool, NORM, EF |
| | liver, primary biliary cirrhosis, 63F, RP |
| | liver/spleen, fetal, 20wM, NORM, CGAP/WM/WN |
| | liver/spleen, fetal, 20wM, NORM, WM |
| | lung tumor line, NCI-H69, sm cell CA, 55M, untreated, WM/WN |
| | lung tumor, adenoCA, 63M |
| | lung tumor, neuroendocrine carcinoid, pool, NORM, 3′ CGAP |
| | lung tumor, neuroendocrine carcinoid, pool, SUB, CGAP |
| | lung tumor, squamous CA, 50M |
| | lung tumor, squamous cell CA, 50M |
| | lung tumor, squamous cell CA, 57M |
| | lung tumor, squamous cell CA, pool, 3′, CGAP |
| | lung tumor, squamous cell CA, pooled, NORM, CGAP |
| | lung, 2M |
| | lung, 72M, WM/WN |
| | lung, asthma, 8F |
| | lung, aw/anencephaly, fetal, 20wF |
| | lung, aw/polycystic kidneys, fetal, 23wM |
| | lung, idiopathic pulmonary disease, NORM |
| | lung, idiopathic pulmonary disease, SUB |
| | lung, mw/mets osteoSAR, aw/pleura mets, 58M, NORM |
| | lung, mw/spindle cell carcinoid, 15-62M/F, pool, lg cDNA |
| | lymph node tumor, follicular lymphoma, 3′ CGAP |
| | lymph node tumor, mets melanoma, 3′, CGAP |
| | lymph node tumor, mets squamous cell CA, aw/tongue CA, 5RP |
| | lymph node, 11F |
| | lymph node, necrotic, aw/lung squamous cell CA, 67M |
| | lymph nodes, 14F |
| | lymphocytes, activated Th1 cells, 6-hr AB |
| | lymphocytes, activated Th2 cells, 6-hr AB |
| | mammary, epithelial cells, 21F, untreated, NORM |
| | mast cell line, HMC-1, leukemia, 52F, untreated |
| | mesentery tumor, sigmoid, mets mixed-mullerian tumor, 61F |
| | microvascular, dermal, endothelial cells, 18F, untreated |
| | microvascular, dermal, endothelial cells, 22F, Untx |
| | mixed tissues, fetal lung, testis, B-cell, SUB, 3′ CGAP/WN |
| | mixed tissues, includes tumor, M/F, pool, 5RP, EF |
| | mixed tissues, includes tumor, pool, SUB, CGAP |
| | mixed tissues, myometrium, smooth muscle cells, 57M/41F, B |
| | mixed tumor, Wilm's/brain mets, pool, 3′ CGAP |
| | mixed tumor, sarcoma, pool, 3′ CGAP |
| | mixed, (melanocytes, uterus, fetal heart), SUB, CGAP/WM/WN |
| | multiple sclerosis, 46M, NORM, WM/WN |
| | muscle, forearm, mw/intramuscular hemangioma 38F |
| | muscle, psoas, 12M |
| | muscle, skeletal |
| | muscle, skeletal, leg, 19F, GEXP |
| | muscle, skeletal, mw/malignant hyperthermia, WM/WN |
| | neuroganglion tumor, ganglioneuroma, 9M |
| | omentum tumor, mets ovary papillary serous CA, F, CGAP |
| | osteogenic tumor line, Saos-2, SAR, 11F, untreated |
| | ovary tumor, dermoid cyst, 22F |
| | ovary tumor, endometrioid CA, 62F |
| | ovary tumor, mets colon adenoCA, 58F |
| | ovary tumor, mixed cancer types, F, NORM, CGAP |
| | ovary tumor, mixed types, pooled, F, 3′ CGAP |
| | ovary, aw/leiomyomata, 36F |
| | ovary, aw/menorrhagia, 47F, 5RP |
| | ovary, endometriosis, aw/leiomyomata, 39F, NORM, EF |
| | pancreas tumor, adenoCA, 3′ CGAP |
| | pancreas, 8M, 5RP |
| | pancreatic tumor, adenoCA, 65F, m/PANCNOT08 |
| | pancreatic tumor, anaplastic CA, 45F |
| | parathyroid tumor, adenoma, M/F, NORM, WM |
| | parathyroid, hyperplasia, 69F |
| | penis tumor, squamous cell CA, 64M |
| | penis, corpora cavernosa, M |
| | penis, corpora cavernosa, aw/scrotal urothelial CA, M |
| | penis, corpus cavernosum, M |
| | penis, corpus cavernosum, mw/CA, 53M |
| | periph blood, B-lymphocytes, CLL, pool, NORM, 3′ CGAP |
| | periph blood, eosinophils, asthma, M/F |
| | periph blood, eosinophils, hypereosinophilia, 48M |
| | periph blood, eosinophils, t/IL-5 |
| | periph blood, lymphocytes, non-adher PBMC, M/F, 24-hr MLR |
| | periph blood, macrophages, adher PBMC, M/F, 72-hr MLR |
| | periph blood, monocytes, 42F, t/IL-10, LPS |
| | periph blood, monocytes, 42F, t/IL-10, LPS, NORM |
| | periph blood, monocytes, 42F, t/antiIL-10, LPS |
| | periph blood, promonocyte line, THP-1, AML, 1M, t/tuberculosis |
| | periph blood, promonocyte line, THP-1, AML, control |
| | periph blood, promonocyte line, THP-1, AML, t/5AZA, SUB |
| | periph blood, promonocyte line, THP-1, AML, t/PMA |
| | periph blood, promonocyte line, THP-1, AML, t/PMA, LPS |
| | periph blood, promonocyte line, THP-1, AML, untreated |
| | peritoneum tumor, neuroendocrine CA, 66F |
| | pituitary gland, aw/AD, mets adenoCA, 70F, RP |
| | pituitary gland, aw/schizophrenia, COPD, 55M, NORM |
| | placenta, aw/hydrocephalus, fetal, 16w, 5RP |
| | placenta, aw/hydrocephalus, fetal, 16w, RP |
| | placenta, aw/hydrocephalus, fetal, 16w, lg cDNA |
| | placenta, aw/hydrocephalus, fetal, 16w/18wM, pool, RP |
| | placenta, fetal, 8-9w, pool, NORM, CGAP/WM |
| | placenta, fetal, M, WM |
| | prostate tumor line, LNCaP, CA, 50M, untreated, 5C-RP |
| | prostate tumor, adenoCA, 57M, m/PROSNOT06 |
| | prostate tumor, adenoCA, 58, 61, 66, 68M, pool, SUB |
| | prostate tumor, adenoCA, 58M |
| | prostate tumor, adenoCA, 59M, SUB, m/PROSNOT19 |
| | prostate tumor, adenoCA, 60M, m/PROSNOT14 |
| | prostate tumor, adenoCA, 65M, m/PROSNOT20 |
| | prostate tumor, adenoCA, 66M, m/PROSNOT15, PROSDIN01 |
| | prostate tumor, adenoCA, 67M, m/PROSNOT05 |
| | prostate tumor, adenoCA, M, m/PROSNOT28/PROSTMC01/PROSTMT05 |
| | prostate, 28M |
| | prostate, 28M, NORM |
| | prostate, AH, mw/adenoCA, 53M |
| | prostate, AH, mw/adenoCA, 57M, 5RP |
| | prostate, AH, mw/adenoCA, 57M, m/PROSTUT04 |
| | prostate, AH, mw/adenoCA, 66M |
| | prostate, AH, mw/adenoCA, 67M |
| | prostate, AH, mw/adenoCA, node mets, 55M, lg/N, m/PROSTUT16 |
| | seminal vesicle, aw/adenoCA, 56M |
| | seminal vesicle, aw/adenoCA, 58M |
| | seminal vesicle, aw/adenoCA, 61M |
| | skin, foreskin, melanocytes, M, NORM, WM/WN |
| | skull tumor, chondroid chordoma, 30F |
| | sm intestine tumor, ileum, mets endometrial adenoCA, 64F |
| | sm intestine, 8M |
| | sm intestine, aw/Patau's syndrome, fetal, 20wM, 5RP |
| | sm intestine, duodenum, 8F |
| | sm intestine, fetal, 20wF |
| | sm intestine, fetal, M, 5RP |
| | sm intestine, ileum, 8F |
| | soft tissue tumor, spinal schwannoma, 35M |
| | soft tissue tumor, thigh, mets myxoid lipoSAR, 34F |
| | spinal cord, aw/renal failure, 71M, NORM |
| | spinal cord, cervical, aw/lymphoma, 32M |
| | spleen tumor, malignant lymphoma, 28M, 5RP |
| | spleen, 2M |
| | spleen, ITP, 14M |
| | spleen, ITP, 8, 14M, pool, lg cDNA |
| | spleen, fetal, 23wM |
| | stomach tumor, lymphoma, 68F |
| | stomach, 55M |
| | synovium, elbow, rheuA, 51F |
| | synovium, hip, rheuA, 68F |
| | synovium, knee, rheuA, 62F |
| | synovium, wrist, dorsal, rheuA, 64F |
| | teratoCA line, NT2, Untx, NORM |
| | teratoCA line, NT2, t/5AZA-3d |
| | teratoCA line, NT2, t/cytokines, 5C-FL, EF |
| | teratoCA line, hNT2, t/RA |
| | teratoCA line, hNT2, t/RA, WM/WN |
| | teratoCA line, hNT2, t/mouse leptin, 9cis RA-6d, lg cDNA |
| | teratoCA line, hNT2, t/mouse leptin, RA |
| | teratoCA line, hNT2, untreated |
| | teratoCA line, hNT2, untreated, WM/WN |
| | testis tumor, M, TIGR |
| | testis tumor, embryonal CA, 31M, 5RP |
| | testis tumor, embryonal CA, 31M, EF |
| | testis, 10-61M, pool, lg cDNA |
| | testis, 16M |
| | testis, 16M, NORM |
| | testis, 26M |
| | testis, M, NORM, CGAP/WN |
| | testis, aw/cirrhosis, 37M |
| | thymus, 3M |
| | thymus, 3M, NORM |
| | thymus, aw/Down, 4mM |
| | thymus, aw/congenital heart abnormalities, 2F |
| | thymus, aw/patent ductus arteriosus, 3M, 5RP |
| | thymus, fetal, pool, NORM, CGAP |
| | thymus, hyperplasia, aw/myasthenia gravis, 16F |
| | thyroid, lymphocytic thyroiditis, mw/papillary CA, 13F |
| | thyroid, mw/follicular adenoma, 28F |
| | thyroid, mw/medullary, papillary CA, node mets, 56M |
| | tonsil, B-lymphocytes, germinal, aw/tonsillitis, NORM, CGAP |
| | tonsil, lymphoid hyperplasia, 6M |
| | umb cord blood, mononuclear cells, M/F, t/G-CSF, lg cDNA |
| | umb cord blood, mononuclear cells, t/IL-5 |
| | umb cord vein endothelial line, HUV-EC-C, shear stress |
| | ureter tumor, TC CA, 64M, 5RP |
| | uterus tumor, F, TIGR |
| | uterus tumor, endometrial adenoCA, F, pooled, 3′ CGAP |
| | uterus tumor, leiomyoma, m/UTRSNON03, UTRSNOT12, UTRSTMR01 |
| | uterus tumor, serous papillary CA, F, pooled, 3′ CGAP |
| | uterus, aw/liver & breast cancer, 46F |
| | uterus, cervix, cervicitis, mw/leiomyoma, 29F, 5RP |
| | uterus, endometrium, F, pool |
| | uterus, endometrium, aw/cystocele, 38F |
| | uterus, endometrium, mw/cervical dysplasia, 32F |
| | uterus, endometrium, mw/leiomyoma, 29F, NORM |
| | uterus, endometrium, type I defect, 28F |
| | uterus, endometrium, type I defect, 30, 32, 36F, pool |
| | uterus, endometrium, type II defect, endometriosis, F |
| | uterus, myometrium, 43F |
| | uterus, myometrium, mw/leiomyoma, 41F, RP, m/UTRSTUT05 |
| | kidney |
| | brain, aw/hypoplastic left heart, fetal, 23wM, FL-EN, EF |
| #39227: | non small cell lung cancer(A-549) |
| | Colon, adenocarcinoma |
| | colon, normal/pseudopolyp, Crohn, 16, 26M, pool, lg cDNA |
| | bone tumor line, MG-63, osteoSar, 14M, untreated |
| | lymph node tumor, axillary, Hodgkin's disease, 45M |
| | synovium, rheuA, 75M/56F, pool, NORM |
| | colon, epithelium, 13F |
| | colon tumor, cecum, adenoCA, 45F |
| | lymph node, 16mM |
| | sm intestine, aw/Patau's syndrome, fetal, 20wM, 5RP |
| | Eye, normal, pigmented retinal epithelium |
| | (HT1080/Dr4) |
| | adrenal gland, 17M |
| | adrenal gland, 20M |
| | adrenal gland, 8M |
| | adrenal gland, aw/pituitary neoplasm, 61F |
| | adrenal gland, mixed, Nrml/pheochromocytoma, pool, lg cDNA, EF |
| | adrenal tumor, adrenal cortical CA, 49M, 5RP, EF |
| | adrenal tumor, cortical CA, aw/hilar mets, 52M, RP |
| | aorta, 64M, WN |
| | aorta, adventitia, 48M |
| | aorta, endothelial cells, M |
| | bladder tumor, TC CA, 60M, m/BLADNOT05 |
| | bladder tumor, TC CA, 80F, m/BLADNOT03 |
| | bladder, 11M, RP |
| | bladder, mw/TC CA, aw/node mets, 58M, m/BLADTUT03 |
| | brain tumor, frontal, astrocytoma, 17F |
| | brain tumor, frontal, mets hypernephroma, 58M |
| | brain tumor, posterior fossa, meningioma, 70M |
| | brain, allocortex, cingulate, aw/cholangioCA, 55F, RP |
| | brain, aw/muscular atrophy, 3mF, NORM, GEXP |
| | brain, frontal cortex, aw/CHF, 35M, NORM |
| | brain, globus pallidus/substantia innominata, aw/CHF, 35M |
| | brain, hippocampus, AD |
| | brain, neocortex, frontal, aw/cholangioCA, 55F, RP |
| | brain, neocortex, parietal, aw/cholangioCA, 55F, RP |
| | brain, pons, Huntington's, mw/CVA, 57M |
| | brain, substantia nigra, aw/atherosclerosis, CHF, 81F, NORM |
| | brain, temporal cortex, aw/aortic aneurysm, 45F |
| | breast tumor, adenoCA, 54F, m/BRSTNOT03 |
| | breast tumor, ductal CA, 68F |
| | breast, F, NORM, WM |
| | breast, PF changes, mw/ductal adenoCA, 54F, m/BRSTTUT02 |
| | breast, mw/ductal CA, CA in situ, aw/node mets, 62F |
| | breast, mw/ductal adenoCA, 46F, m/BRSTTUS08, BRSTTUT13 |
| | breast, mw/lobular CA, 67F |
| | cervical tumor line, HeLa, adenoCA, 31F, untreated |
| | clavicle, osteoblasts, 40M, untreated, lg cDNA, EF |
| | colon tumor, adenoCA, 3′, CGAP |
| | colon tumor, adenoCA, 75M, m/COLNNOT01 |
| | colon tumor, adenoCA, NORM, 3′ CGAP |
| | colon tumor, sigmoid, adenoCA, 62M, m/COLNNOT16 |
| | colon tumor, villous adenoma, 3′ CGAP |
| | colon, CUC, 69M |
| | colon, ascending, CUC, 32M |
| | colon, aw/anencephaly, fetal, 20wF |
| | colon, aw/gastroparesis, 37F |
| | colon, cecum, Crohn's, 31M |
| | colon, cecum, benign familial polyposis, 16M |
| | colon, epithelium, 13F, RP |
| | colon, mucosa, Crohn, pool, NORM, CGAP |
| | colon, mw/adenoCA, 55M, m/COLHTUT01, COLHTUS02 |
| | colon, mw/adenoCA, aw/COPD, 75M, m/COLNTUT02 |
| | colon, normal/pseudopolyp, Crohn, 16, 26M, pool, lg/N |
| | colon, transverse, benign familial polyposis, 16M |
| | esophagus, aw/adenoCA, 61M |
| | eye, retina, 55M, NORM, WM |
| | fallopian tube tumor, endometrioid/serous adenoCA, 85F, RP |
| | fat, abdomen, aw/obesity, 52F |
| | fibroblasts, senescent, NORM, WM/WN |
| | gallbladder tumor, squamous cell CA, 78F |
| | gallbladder, cholecystitis, cholelithiasis, 21M |
| | gallbladder, cholecystitis, cholelithiasis, 53F |
| | gallbladder, cholecystitis, cholelithiasis, 55F, 5RP |
| | ganglion, dorsal root, thoracic, aw/lymphoma, 32M, NORM |
| | ganglion, dorsal root, thoracic/lumbar, aw/lymphoma, 32M |
| | heart, left ventricle, 31M |
| | kidney tumor, clear cell type cancer, pool, NORM, 3′ CGAP |
| | kidney tumor, renal cell CA, 46M, 5RP |
| | kidney tumor, renal cell CA, 51F |
| | kidney, fetal, 23wM |
| | kidney, interstitial nephritis, aw/bladder TC CA, 63M, 5RP |
| | liver tumor line, C3A, Hepatob, 15M, t/MCA, SUB |
| | liver tumor line, C3A, Hepatob, 15M, t/MCA-48 hr |
| | liver tumor, mets colon adenoCA, 51F |
| | liver/spleen, fetal, 20wM, NORM, CGAP/WM/WN |
| | lung tumor, adenoCA, 63M |
| | lung tumor, mets thyroid CA, 79M, m/LUNGNOT03 |
| | lung tumor, squamous CA, 50M |
| | lung tumor, squamous cell CA/mets liposarcoma, pool, SUB, EF |
| | lung, 12M |
| | lung, 15F |
| | lung, 47M |
| | lung, NORM |
| | lung, aw/anencephaly, fetal, 17wF |
| | lung, aw/anencephaly, fetal, 20wF |
| | lung, mw/endobronchial carcinoid, 33M |
| | lung, mw/mets osteoSAR, aw/pleura mets, 58M, NORM |
| | lung, panacinar emphysema, mw/granuloma, 61M |
| | lymph nodes, 16mM, NORM |
| | lymphocytes, PBMC, M, 96-hr MLR |
| | mixed tissues, fetal lung, testis, B-cell, SUB, 3′ CGAP/WN |
| | mixed tissues, myometrium, smooth muscle cells, 57M/41F, B |
| | nasal/cribriform tumor, olfactory neuroblastoma, 45M, 5RP |
| | nose, olfactory epithelium, 35F, WM |
| | ovary tumor, dermoid cyst, 22F |
| | ovary tumor, mets colon adenoCA, 58F |
| | ovary, aw/leiomyoma, 47F |
| | ovary, aw/leiomyomata, 52F |
| | ovary, aw/menorrhagia, 47F, 5RP |
| | ovary, mw/mucinous cystadenoCA, 43F, m/OVARTUT01 |
| | ovary, stromal hyperthecosis, mw/dermoid cyst, 45F, RP |
| | pancreas tumor, adenoCA, 3′ CGAP |
| | pancreas, 17F, NORM |
| | pancreas, 5M |
| | pancreas, islet cell hyperplasia, 15M |
| | pancreatic tumor line, adenoCA, untreated, WM/WN |
| | pancreatic tumor, adenoCA, 65F, m/PANCNOT08 |
| | penis tumor, squamous cell CA, 64M |
| | periph blood, eosinophils, asthma, M/F |
| | periph blood, macrophages, adher PBMC, M/F, 48-hr MLR |
| | periph blood, monocytes, 42F, t/IL-10, LPS, NORM |
| | periph blood, promonocyte line, THP-1, AML, untreated |
| | pituitary gland, aw/AD, mets adenoCA, 70F, RP |
| | placenta, fetal, 18wM |
| | placenta, fetal, 8-9w, pool, NORM, CGAP/WM |
| | probable cartilage, knee, chondrocytes, M/F, t/IL-1 |
| | prostate tumor, adenoCA, 59M, SUB, m/PROSNOT19 |
| | prostate tumor, adenoCA, 59M, m/PROSNOT19 |
| | prostate tumor, adenoCA, 66M, m/PROSTMT02 |
| | prostate, 32M, SUB, 3′ CGAP |
| | prostate, AH, mw/adenoCA, 57M, 5RP |
| | prostate, AH, mw/adenoCA, 57M, m/PROSTUT04 |
| | renal vein, smooth muscle cells, 57M, Untx |
| | seminal vesicle, aw/prostate adenoCA, 63M, 5RP |
| | skin, dermis, breast, fibroblasts, 31F, t/9CIS RA, lg cDNA, EF |
| | skin, dermis, breast, fibroblasts, 31F, t/9CIS RA-20 hr |
| | skin, epidermal breast keratinocyte line, NHEK, 30F, Untx |
| | sm intestine, 13M |
| | sm intestine, 31F, RP |
| | sm intestine, duodenum, aw/pancreatic cystadenoma, 41F |
| | sm intestine, ileum, Crohn's, 18F |
| | sm intestine, ileum, aw/adenoCA cecum, node mets, 70F, 5RP |
| | sm intestine, ileum, mw/CUC, 42M |
| | sm intestine, ileum, mw/carcinoid, adenoCA, F, pool, lg cDNA |
| | sm intestine, jejunum, 16M |
| | sm intestine, mw/carcinoid, aw/node mets, 59M |
| | spleen tumor, Hodgkin's, 45M |
| | spleen tumor, malignant lymphoma, 28M, 5RP |
| | spleen, 2M |
| | spleen, ITP, 8, 14M, pool, lg cDNA |
| | spleen, fetal, 23wM |
| | stomach tumor, adenoCA, poorly differentiated, 3′ CGAP |
| | stomach tumor, pool, 3′ CGAP |
| | stomach, gastritis, mw/adenoCA, node mets, 76M, RP |
| | synovium, elbow, rheuA, 51F |
| | synovium, wrist, dorsal, rheuA, 64F |
| | thymus tumor, malignant thymoma, 56F |
| | thymus, fetal, M |
| | thyroid tumor, follicular adenoma, 17M |
| | thyroid, AH, mw/papillary CA, 22F, 5RP, EF |
| | thyroid, mw/follicular adenoma, 28F |
| | umb cord blood, mononuclear cells |
| | uterus tumor, endometrial adenoCA, F, pooled, 3′ CGAP |
| | uterus tumor, endometrium, adenosquamous CA, 49F, 5RP |
| | uterus tumor, leiomyoma, 37F, 5RP |
| | uterus tumor, leiomyomata/adenosquamousCA, F, pool, lg cDNA, EF |
| | uterus, F, NORM, CGAP/WM/WN |
| | uterus, aw/ovarian follicular cysts, 34F |
| | uterus, cervix, cervicitis, mw/leiomyoma, 29F, 5RP |
| | uterus, endometrium, mw/cervical dysplasia, 32F |
| | uterus, endometrium, type I defect, 28F |
| | uterus, endometrium, type II defect, endometriosis, F |
| | uterus, mw/leiomyoma, aw/colon adenoCA, 45F |
| | uterus, myometrium, mw/adenoCA, 59F, RP |
| | uterus, myometrium, mw/leiomyoma, 41F, NORM, m/UTRSTUT05 |
| | uterus, soft tissue, mw/leiomyomata, 33F |
| | white blood cells, 27F |
| | CML precursor line, K-562, 53F, Untx |
| | PBMC, 60M, untreated |
| | PBMC, M/F, pool, untreated |
| | T-lymphocytes, CD4+, pool, t/anti-CD3 antibodies |
| | T-lymphocytes, allogenic anergic, 40-50M, t/OKT3 3 day |
| | T-lymphocytes, allogenic, 40-50M, t/OKT3 6 hr |
| | T-lymphocytes, allogenic, 40-50M, untreated |
| | adrenal gland, aw/renal cell CA, 43M |
| | adrenal gland, mw/pheochromocytoma, 43F, m/ADRETUT07 |
| | adrenal glands, aw/anencephaly, fetal, 16wF, lg cDNA |
| | adrenal tumor, mets renal cell CA, 50M |
| | adrenal tumor, pheochromocytoma, 52F, EF |
| | aorta, endothelial cells, 33F, treated |
| | aorta, endothelial cells, TIGR |
| | aortic smooth muscle line, M |
| | astrocytes, M/F, t/cytokines 12 hr |
| | astrocytes, M/F, t/cytokines 4-6 hr |
| | bladder tumor, TC CA, 58M |
| | bladder tumor, TC CA, 58M, m/BLADNOT09 |
| | bladder tumor, TC CA, 72M |
| | bladder, 11M |
| | bone marrow, tibia, aw/mets alveolar rhabdomyoSAR, 16M, EF |
| | bone, trabecula/femoral neck, 66M, t/DEXA, BMP-2-48 hr |
| | brain stem, aw/DMt1, 72M |
| | brain stem, aw/DMt1, 72M, NORM |
| | brain tumor, frontal, meningioma, 61F |
| | brain tumor, frontal, mets hypernephroma, 58M, 5RP |
| | brain tumor, glioblastoma, pool, NORM, CGAP |
| | brain tumor, medulloblastoma, NORM, 3′ CGAP |
| | brain, aw/hypoplastic left heart, fetal, 23wM, NORM |
| | brain, choroid plexus, Huntington's, mw/CVA, 57M |
| | brain, hippocampus, AD, 74M |
| | brain, hippocampus, aw/CHF, 35M |
| | brain, hippocampus, aw/CHF, 35M, NORM |
| | brain, medulla, ALS, 74F |
| | brain, occipital, Huntington's, mw/CVA, 57M |
| | brain, pineal gland, aw/AD, 68, 79F, pool, NORM |
| | brain, temporal, gliosis, mw/epilepsy, 27M, lg cDNA |
| | brain, thalamus, aw/CHF, 35M, NORM |
| | breast tumor, adenoCA, 45F, m/BRSTNOT09 |
| | breast tumor, adenoCA, 46F, m/BRSTNOT33, EF |
| | breast tumor, ductal CA, 65F |
| | breast tumor, ductal adenoCA, 66F |
| | breast tumor, high vascular density, CA, F |
| | breast tumor, lobular CA, 58F, m/BRSTNOT05 |
| | breast, 46, 60F, pool, NORM |
| | breast, NF changes, mw/ductal adenoCA, 40-57F, pool, lg cDNA |
| | breast, PF changes, 40F |
| | breast, PF changes, mw/adenoCA, 45F, m/BRSTTUT08 |
| | breast, PF changes, mw/adenoCA, 55F, m/BRSTTUT01 |
| | breast, PF changes, mw/adenoCA, intraductal CA, 43F |
| | breast, mw/ductal CA, 43F, m/BRSTTUT16 |
| | breast, mw/ductal adenoCA, aw/node mets, 46F, m/BRSTTUT15 |
| | breast, mw/ductal adenoCA, intraductal CA, aw/node mets, 57F |
| | bronchial epithelium line, NHBE, 54M, Untx, NORM |
| | cartilage, OA |
| | cartilage, knee, chondrocytes, M/F, t/IL-1 |
| | colon polyp, aw/adenoCA, tubulovillous adenoma, 40F |
| | colon tumor, adenoCA, carcinoid, M/F, pool, NORM |
| | colon tumor, adenoCA, pool, NORM, 3′ CGAP |
| | colon tumor, adenoCA, pool, NORM, 3′/5′ CGAP |
| | colon tumor, rectum, adenoCA, mw/tubular adenoma, 50M, 5RP |
| | colon, 16M |
| | colon, ascending, CUC, 25F |
| | colon, ascending, CUC, 74M, EF |
| | colon, cecum polyp, aw/adenoCA, 67F |
| | colon, cecum, mw/Crohn's, 18F |
| | colon, descending, benign familial polyposis, 16M |
| | colon, mw/Crohn's, 56F |
| | colon, mw/adenoCA, aw/node mets, 60M, m/COLNTUT16 |
| | colon, sigmoid, mw/adenoCA, aw/node mets, 62M, m/COLNTUT03 |
| | colon, ulcerative colitis, 16M |
| | esophagus tumor, squamous cell CA, 3′, CGAP |
| | fallopian tube tumor, endometrioid/serous adenoCA, 85F |
| | fat, mesentery, aw/diverticulosis, diverticulitis, 71M |
| | fibroblasts, aortic adventitia, 48M, untreated |
| | fibroblasts, aortic adventitia, 65F, untreated |
| | ganglion, dorsal root, cervical, aw/lymphoma, 32M |
| | ganglion, dorsal root, cervical, aw/lymphoma, 32M, NORM, EF |
| | heart tumor, left atrium, myxoma, 43M |
| | heart, coronary artery, CAD, 46M |
| | heart, fetal, 18wM |
| | heart, left ventricle, mw/myocardial infarction, 56M |
| | heart, right atrium/muscle wall, Pompe's, 7mM |
| | iliac artery, endothelial cells, F, control, untreated |
| | iliac artery, endothelial cells, F, untreated |
| | kidney tumor, renal cell CA, 53F, m/KIDNNOT26 |
| | kidney tumor, renal cell CA, 65M m/KIDNNOT19 |
| | kidney tumor, renal cell, 3′ CGAP |
| | kidney, 49M |
| | kidney, 64F |
| | kidney, cortex, mw/renal cell CA, 65M, 5RP |
| | kidney, medulla/cortex, mw/renal cell CA, 53F, m/KIDNTUT16 |
| | kidney, mw/renal cell CA, 65M, m/KIDNTUT15 |
| | kidney, mw/renal cell CA, 8, 53F, pool, NORM |
| | kidney, pool, SUB, 3′ CGAP |
| | liver, aw/Patau's, anencephaly, fetal, pool, lg cDNA |
| | liver, pool, NORM, EF |
| | lung tumor, adenoCA, 47M |
| | lung tumor, adenoCA, 53M, m/LUNGNOT28 |
| | lung tumor, adenoCA, 70F |
| | lung tumor, squamous cell CA, 50M |
| | lung tumor, squamous cell CA, 68M |
| | lung, 2M |
| | lung, 35F, 5RP |
| | lung, aw/polycystic kidneys, fetal, 23wM |
| | lung, mw/adenoCA, 66F |
| | lung, mw/adenoCA, COPD, 47M |
| | lung, mw/adenoCA, aw/node, diaphragm mets, 63F |
| | lung, mw/mets thyroid CA, 79M, m/LUNGTUT02 |
| | lung, mw/spindle cell carcinoid, 15-62M/F, pool, lg cDNA |
| | lung, pleura, aw/mets leiomyoSAR to lung, 58F, lg cDNA |
| | lung, pneumonitis, mw/squamous cell CA, 69M, m/LUNGTUT03 |
| | lymph node tumor, Hodgkin's/mets adenoCA, F, pool, lg cDNA |
| | lymph node tumor, follicular lymphoma, 3′ CGAP |
| | lymph nodes, 14F |
| | lymph nodes, 42F |
| | mast cells, liver, fetal, 22w |
| | microvascular, dermal, endothelial cells, 18F, untreated |
| | microvascular, dermal, endothelial cells, 22F, Untx |
| | microvascular, dermal, endothelial cells, neonatal, M |
| | muscle, forearm, mw/intramuscular hemangioma 38F |
| | muscle, glossal, mw/squamous cell CA, 41F |
| | muscle, skeletal, aw/Krabbe, 11mF |
| | neuroganglion tumor, ganglioneuroma, 9M |
| | nose, nasal polyps, aw/asthma, 78M, pool, NORM |
| | nose, nasal polyps, lg cDNA, EF |
| | ovary tumor, adenoCA, 58F |
| | ovary tumor, mets colon adenoCA, 58F, NORM |
| | ovary tumor, mixed types, pooled, F, 3′ CGAP |
| | ovary tumor, serous CA, mets colon CA, 44F, 5RP, EF |
| | ovary, aw/leiomyomata, 36F |
| | ovary, aw/leiomyomata, 36F, NORM |
| | ovary, cystic, aw/cystadenoma, 34F |
| | ovary, endometriosis, aw/leiomyomata, 39F |
| | pancreas, 29M |
| | pancreas, 2M |
| | pancreas, 8M, 5RP |
| | pancreas, islet cells, pool |
| | pancreas, type I diabetes, 43F |
| | pancreatic tumor, anaplastic CA, 45F |
| | paraganglion tumor, paraganglioma, aw/CA, 46M, NORM |
| | penis, corpus cavernosum, M |
| | periph blood, B-lymphocytes, CLL, pool, NORM, 3′ CGAP |
| | periph blood, eosinophils, t/IL-5 |
| | periph blood, granulocytes, M/F, t/GM-CSF |
| | periph blood, granulocytes, M/F, t/LPS |
| | periph blood, lymphocytes, non-adher PBMC, 24M |
| | periph blood, macrophages, adher PBMC, M/F, 72-hr MLR |
| | periph blood, macrophages, adher PBMC, M/F, MLR-24 hr |
| | periph blood, monocytes, 42F, t/IL-10, LPS |
| | periph blood, monocytes, 42F, t/antiIL-10, LPS |
| | periph blood, monocytes, 42F, t/antiIL-10, LPS, NORM |
| | periph blood, promonocyte line, THP-1, AML, stimulated |
| | pituitary gland, aw/schizophrenia, COPD, 55M, NORM |
| | placenta, aw/hydrocephalus, fetal, 16w |
| | prostate stroma, fibroblasts, fetal, 26wM, untreated |
| | prostate tumor, TC CA, 66M, EF |
| | prostate tumor, adenoCA, 59M, SUB, m/PROSNOST19 |
| | prostate tumor, adenoCA, 60M, m/PROSNOT14 |
| | prostate tumor, adenoCA, M, m/PROSNOT28/PROSTMC01/PROSTMT05 |
| | prostate, AH, aw/bladder TC CA, 58M |
| | prostate, AH, mw/adenoCA, 60M, m/PROSTUT08 |
| | prostate, AH, mw/adenoCA, 65M, m/PROSTUT12 |
| | prostate, AH, mw/adenoCA, 66M, m/PROSTUT17 |
| | prostate, AH, mw/adenoCA, 73M |
| | prostate, AH, mw/adenoCA, 73M, FL-EN, EF |
| | prostate, AH, mw/adenoCA, node mets, 55M, lg/N, m/PROSTUT16 |
| | prostate, mw/adenoCA, 68M, m/PROSTUT18 |
| | renal vein, smooth muscle cells, 57M t/TNF, IL1 |
| | seminal vesicle, aw/adenoCA, 58M |
| | seminal vesicle, aw/adenoCA, 61M |
| | skin, breast, aw/adenoCA, 70F |
| | skin, breast, aw/adenoCA, 70F, lg cDNA |
| | skin, dermis, breast, fibroblasts, 31F, untreated |
| | skin, leg, erythema nodosum |
| | sm intestine tumor, ileum, mets ovarian adenoCA, 49F |
| | sm intestine, 8M |
| | sm intestine, aw/stomach ulcer, 49F, 5RP |
| | sm intestine, duodenum, 16M |
| | sm intestine, ileum, aw/adenoCA cecum, node mets, 70F |
| | sm intestine, ileum, mw/carcinoid, 30F |
| | soft tissue tumor, spinal schwannoma, 35M |
| | soft tissue tumor, thigh, mets myxoid lipoSAR, 34F |
| | spinal cord, aw/renal failure, 71M, NORM |
| | spleen, 29M |
| | spleen, 8M |
| | spleen, Gaucher's, 22M |
| | stomach, fetal, 18wM |
| | stomach, fetal, 20wF |
| | synovium, 75M |
| | synovium, knee, rheuA, 62F |
| | synovium, wrist, rheuA, 56F |
| | synovium, wrist, rheuA, 62F |
| | teratoCA line, NT2, t/5AZA-3d |
| | testis, 16M, NORM |
| | testis, M, NORM, CGAP/WN |
| | testis, necrosis, 31M |
| | thymus, 3M |
| | thymus, aw/parathyroid adenoma, 21M |
| | thyroid, lymphocytic thyroiditis, mw/papillary CA, 30F |
| | thyroid, mw/medullary, papillary CA, node mets, 56M |
| | tongue tumor, squamous cell CA, 36M |
| | tonsil, lymphoid hyperplasia, 6M |
| | tonsil, lymphoid hyperplasia, 6M/9F, pool, lg cDNA, EF |
| | umb cord blood, mononuclear cells, M/F, t/G-CSF |
| | umb cord blood, mononuclear cells, M/F, t/G-CSF, lg cDNA |
| | umbilical vein, endothelial cells, untreated |
| | uterus tumor, F, TIGR |
| | uterus tumor, leiomyoma, 41F |
| | uterus tumor, serous papillary CA, F, pooled, 3′ CGAP |
| | uterus, aw/liver & breast cancer, 46F |
| | uterus, cervix, cervicitis, mw/leiomyoma, 29F, lg cDNA, EF |
| | uterus, endometrium, aw/cystocele, 38F |
| | uterus, endometrium, mw/cervical dysplasia, 32F, 5RP |
| | uterus, endometrium, mw/cervicitis, leiomyoma, pool, lg cDNA |
| | uterus, endometrium, type I defect, 30, 32, 36F, pool |
| | uterus, myometrium, mw/leiomyoma, 41F, m/UTRSTUT05 |
| | white blood cells, 45F |
| | kidney |
| | brain tumor, frontal, meningioma, 50M |
| | esophagus tumor, adenoCA, 61M, 5RP, EF |
| | heart, right atrium, 39M |
| | kidney, cortex, mw/renal cell CA, 65M |
| | stomach, aw/esophagus adenoCA, 61M, 5RP |
| | tonsil, lymphoid hyperplasia, 6M, 5RP, EF |
| | brain tumor, frontal, neuronal neoplasm, 32M |
| | lung, 17F |
| | lung, mw/adenoCA, COPD, 78M |
| | sm intestine tumor, ileum, carcinoid, 42M |
| #38645: | Uterus, endometrium adenocarcinoma |
| | signet-ring cell carcinoma(KATO III) |
| | aorta, 64M, WN |
| | brain tumor, frontal, neuronal neoplasm, 32M |
| | mixed, (melanocytes, uterus, fetal heart), SUB, CGAP/WM/WN |
| | heart, left ventricle, 51F |
| | CML precursor line, K-562, 53F, Untx |
| | Jurkat line, T-cell leukemia, M, t/PMA, Iono-1 hr, 5C-FL, EF |
| | Jurkat line, T-cell leukemia, M, untreated |
| | adrenal gland, mw/pheochromocytoma, 43F, m/ADRETUT07 |
| | adrenal tumor, pheochromocytoma, 52F, EF |
| | aorta, adventitia, 65F |
| | aorta, endothelial cells, 33F, treated |
| | bladder, mw/TC CA, aw/prostate TC CA, 66M, m/BLADTUT05 |
| | bone tumor, rib, mets osteoSAR, 16M |
| | brain tumor, anaplastic oligodendroglioma, pool, NORM, CGAP |
| | brain tumor, frontal, mets hypernephroma, 58M |
| | brain tumor, glioblastoma, pool, NORM, CGAP |
| | brain, aw/hypoplastic left heart, fetal, 23wM, FL-EN, EF |
| | brain, frontal, fetal, 5mM, 3′/5′, WN |
| | brain, temporal, gliosis, mw/epilepsy, 27M, lg cDNA |
| | breast tumor, ductal CA, 43F, m/BRSTTMT01 |
| | breast, NF breast disease, 32F |
| | breast, PF changes, mw/adenoCA, 55F, m/BRSTTUT01 |
| | breast, mw/ductal CA, CA in situ, aw/node mets, 62F |
| | breast, mw/ductal adenoCA, 62F, RP |
| | breast, mw/ductal adenoCA, aw/node mets, 46F, m/BRSTTUT15 |
| | breast, mw/neoplasm, 36F |
| | colon tumor, adenoCA, 3′, CGAP |
| | colon tumor, adenoCA, pool, NORM, 3′/5′ CGAP |
| | colon tumor, hepatic flexure, adenoCA, 55M, m/COLATMT01 |
| | colon, descending, CUC, 28M, 5RP |
| | colon, epithelium, 13F |
| | colon, mw/adenoCA, aw/node mets, 60M, m/COLNTUT16 |
| | epididymis tumor, CA, M, pool, LICR, EF |
| | esophagus tumor, adenoCA, 61M |
| | fallopian tube tumor, endometrioid/serous adenoCA, 85F, RP |
| | fat, mixed tissues, aw/breast adenoCA, 38-73M/F, pool, NORM |
| | fibroblasts, aortic adventitia, 48M, untreated |
| | fibroblasts, senescent, NORM, WM/WN |
| | ganglion, dorsal root, cervical, aw/lymphoma, 32M |
| | ganglion, dorsal root, thoracic, aw/lymphoma, 32M, NORM |
| | germ cell tumor, pool, SUB, 3′ CGAP |
| | germ cell tumor, seminoma, teratoma adenoCA, pool, 3′ CGAP |
| | heart, fetal, 19w, NORM, CGAP/WM/WN |
| | heart, right atrium, 51F |
| | iliac artery, endothelial cells, F, control, untreated |
| | kidney tumor, clear cell type cancer, pool, SUB, CGAP |
| | kidney tumor, renal cell, 3′ CGAP |
| | kidney, fetal, 19-23w, M/F, lg cDNA |
| | kidney, pool, SUB, 3′ CGAP |
| | liver, aw/anencephaly, fetal, 16wF |
| | liver/spleen, fetal, 20wM, NORM, CGAP/WM/WN |
| | lung tumor, mets thyroid CA, 79M, m/LUNGNOT03 |
| | lung tumor, neuroendocrine carcinoid, pool, SUB, CGAP |
| | lung tumor, squamous cell CA, pool, 3′, CGAP |
| | lung, 72M |
| | lung, asthma, 17M |
| | lung, idiopathic pulmonary disease, SUB |
| | lung, mw/caseating granuloma, 58F |
| | lung, mw/mets osteoSAR, aw/pleura mets, 58M |
| | lung, right bronchus, asthmatic, 22-51M/F, pool |
| | lymph node tumor, follicular lymphoma, 3′ CGAP |
| | lymph node, 11F |
| | lymph node, mesenteric, 13M, FL-EN, EF |
| | lymph nodes, 14F |
| | lymphocytes, PBMC, M, 96-hr MLR |
| | microvascular, dermal, endothelial cells, 22F, t/VEGF, EF |
| | mixed tissues, fetal lung, testis, B-cell, SUB, 3′ CGAP/WN |
| | mixed tissues, includes tumor, SUB, 3′ CGAP |
| | mouth line, gingiva, keratinocytes, CGAP, EF |
| | muscle tumor, striated, alveolar rhabdomyoSAR, 3′/5′ CGAP |
| | muscle, psoas, 12M |
| | neural tumor, schwannoma, pool, 3′ CGAP |
| | neuroganglion tumor, ganglioneuroma, 9M |
| | osteogenic tumor line, Saos-2, SAR, 11F, untreated |
| | ovary tumor, papillary serous CA, 64F, WM/WN |
| | ovary, endometriosis, aw/leiomyomata, 39F |
| | ovary, mw/follicular cysts, 28F |
| | ovary, mw/mucinous cystadenoCA, 43F, m/OVARTUT01 |
| | pancreas tumor, adenoCA, 3′ CGAP |
| | pancreatic tumor, TIGR |
| | penis tumor, squamous cell CA, 64M |
| | periph blood, B-lymphocytes, CLL, pool, NORM, 3′ CGAP |
| | periph blood, T-lymphocytes, CD8+, 63M, untreated |
| | periph blood, granulocytes, M/F, t/LPS |
| | periph blood, macrophages, adher PBMC, M/F, t/LPS |
| | pituitary gland, aw/AD, mets adenoCA, 70F, RP |
| | pituitary gland, aw/schizophrenia, COPD, 55M |
| | prostate, 21M, TIGR |
| | skin, dermis, breast, fibroblasts, 31F, t/9CIS RA-20 hr, SUB |
| | sm intestine, duodenum, aw/pancreatic cystadenoma, 41F |
| | spinal cord, base medulla, Huntington's, 57M, lg cDNA, EF |
| | synovium, rheuA, 75M/56F, pool, NORM |
| | teratoCA line, hNT2, t/RA, WM/WN |
| | testis tumor line, embryonal CA, pool, MGC, EF |
| | testis, M, NORM, CGAP/WN |
| | thymus, 3M |
| | thymus, aw/congenital heart abnormalities, 2F |
| | thymus, fetal, pool, NORM, CGAP |
| | thymus, hyperplasia, aw/myasthenia gravis, 16F |
| | tonsil, B-lymphocytes, germinal, aw/tonsillitis, NORM, CGAP |
| | tonsil, lymphoid hyperplasia, 6M |
| | tonsil, lymphoid hyperplasia, 6M/9F, pool, AMP |
| | umb cord blood, T-lymphocytes, Th2 cells, untreated |
| | umb cord blood, mononuclear cells |
| | uterus, aw/cardiomyopathy, 59F |
| | uterus, cervix tumor, CA, F, NORM, 3′ CGAP |
| | uterus, endometrium, type I defect, 28F |
| | uterus, myometrium, mw/adenoCA, 59F, RP |
| | uterus, myometrium, mw/leiomyoma, 41F, NORM, m/UTRSTUT05 |
| | uterus, myometrium, mw/leiomyoma, 41F, RP, m/UTRSTUT05 |
| | uterus, tumor line, adenoCA/leiomyoSAR, pool, MGC, EF |
| | breast tumor, ductal, F, pool, NORM, 3′/5′ CGAP |
| | mixed tissues, includes tumor, treated cells, M/F, pool, 5RP, EF |
| | prostate, AH, mw/adenoCA, 67M |
| | spleen, 2M |
| | umbilical vein, endothelial cells, HUVEC, t/TNFa-48 hr, 5C-FL, EF |
| | kidney epithelial transf embryo line, 293-EBNA, t/5AZA |
| #36909: | colon, aw/anencephaly, fetal, 20wF |
| | mixed, (melanocytes, uterus, fetal heart), SUB, CGAP/WM/WN |
| | bladder tumor line, CA/TC CA/papilloma, pool, MGC, EF |
| | bone tumor line, MG-63, osteoSar, 14M, untreated |
| | bone, rib, aw/Patau's, fetal, 20wM |
| | brain, mw/oligoastrocytoma, epilepsy, 26M, NORM |
| | breast, PF changes, mw/adenoCA, intraductal CA, 43F |
| | bronchial epithelium line, NHBE, 54M, Untx |
| | colon, epithelium, 13F, RP |
| | eye, retina, fetal, pool, WM/WN |
| | fetus, 8-9w, pool, NORM, CGAP/WM/WN |
| | heart, fetal, 19w, NORM, CGAP/WM/WN |
| | heart, right atrium/muscle wall, Pompe's, 7mM, RP |
| | kidney, right/left, aw/Patau's, fetal, 20wM, pool, lg cDNA |
| | liver tumor, mets neuroendocrine CA, 72M, 5RP, EF |
| | lung fibroblast line, WI38, 3mF, untreated, WM |
| | lung, aw/polycystic kidneys, fetal, 23wM |
| | lung, idiopathic pulmonary disease, SUB |
| | muscle, skeletal, MGC, EF |
| | neural tumor, schwannoma, pool, 3′ CGAP |
| | osteogenic tumor line, Saos-2, SAR, 11F, untreated |
| | ovary tumor, fibrothecoma, F, pool, NORM, 3′, CGAP |
| | ovary tumor, mucinous cystadenoCA, 43F, m/OVARNOT03 |
| | ovary tumor, seroanaplastic CA, 52F |
| | ovary, aw/leiomyomata, 36F |
| | ovary, aw/leiomyomata, 36F, NORM |
| | ovary, cystic, aw/cystadenoma, 34F |
| | ovary, follicular cysts, 28F |
| | pituitary gland, aw/schizophrenia, COPD, 55M, NORM |
| | pituitary, 15-75M/F, pool |
| | placenta, aw/hydrocephalus, fetal, 16w, RP |
| | placenta, fetal, 18wM |
| | prostate, 28M, NORM |
| | prostate, 32M, SUB, 3′ CGAP |
| | prostate, AH, mw/adenoCA, node mets, 55M, lg/N, m/PROSTUT16 |
| | renal vein, smooth muscle cells, 57M, Untx, 5RP, EF |
| | sm intestine, duodenum tumor line, adenoCA, MCG, EF |
| | smooth muscle tumor, CA/leiomyoSAR, pool, LICR, EF |
| | testis, 16M, NORM |
| | thymus, aw/congenital heart abnormalities, 2F, 5RP |
| | uterus tumor, endometrial adenoCA, F, pooled, 3′ CGAP |
| | uterus, aw/ovarian follicular cysts, 34F |
| | uterus, endometrium, mw/cervical dysplasia, 32F, 5RP |
| | uterus, myometrium, mw/leiomyoma, 41F, NORM, m/UTRSTUT05 |
| #38327: | Caucasian male fetus placenta |
| | brain tumor, frontal, meningioma, 61F |
| | kidney, interstitial nephritis, aw/bladder TC CA, 63M, 5RP |
| | thymus, hyperplasia, aw/myasthenia gravis, 16F |
| | muscle, thigh, mw/lipoSAR, 58M, RP |
| #41822: | EBV-transformed B lymphocyte |
| | T-cell (jurkat) |
| | brain stem |
| | brain, infant, 10wF, NORM, WM |
| | colon tumor, ileocecum, Burkitt lymphoma, 29F, m/COLANOT03 |
| | pancreas, aw/Patau's, fetal, 20wM |
| | prostate tumor, adenoCA, 69M, m/PROSNOT07 |
| | sm intestine, ileum, 4F |
| | uterus, cervix, cervicitis, mw/leiomyoma, 29F, 5RP |
| | bone marrow tumor, CA, pool, LICR, EF |
| | brain, aw/hypoplastic left heart, fetal, 23wM, lg cDNA |
| | mixed tissues, fetal lung, testis, B-cell, SUB, 3′ CGAP/WN |
| | lung, 47M |
| | T-lymphocytes, allogenic anergic, 40-50M, t/OKT3 3 day |
| | fetus, 8-9w, pool, NORM, CGAP/WM/WN |
| | kidney, mw/renal cell CA, 8, 53F, pool, NORM |
| | multiple sclerosis, 46M, NORM, WM/WN |
| | bone marrow, 16-70M/F |
| | brain, astrocytes, fetal, 22wF, t/carbachol 16 hr |
| | brain, thalamus, aw/CHF, 35M |
| | kidney epithelial transf embryo line, 293-EBNA, Untx |
| | lung, pleura, aw/mets leiomyoSAR to lung, 58F, lg cDNA |
| | mixed tissue tumor, head/neck, CA, pool, LICR, EF |
| | muscle, skeletal |
| | peritoneum tumor, neuroendocrine CA, 66F |
| | prostate, mw/adenoCA, 65M |
| | spleen, 2M |
| | testis, 10-61M, pool, lg cDNA |
| | thymus, aw/congenital heart abnormalities, 2F, RP |
| | unspecified tumor, CA, aw/Denys-drash, pool, LICR, EF |
| | nervous tissue tumor, CA, pool, LICR, EF |
| | uterus, endometrium, mw/cervical dysplasia, 32F, FL-EN, EF |
| | CML precursor line, K-562, 53F, Untx, NORM, EF |
| | CML precursor line, K-562, 53F, t/9cis RA-13d |
| | Jurkat line, T-cell leukemia, M, t/PMA |
| | Jurkat line, T-cell leukemia, M, t/PMA, Iono-1 hr, 5C-FL, EF |
| | Jurkat line, T-cell leukemia, M, t/PMA-30 min, 5C-FL, EF |
| | Jurkat line, T-cell leukemia, untreated, TIGR |
| | PBMC, 60M, untreated |
| | T-lymphocyte, activated, TIGR |
| | T-lymphocytes, CD4+, pool, t/anti-CD3, anti-CD28 antibodies |
| | adenoid, inflamed, 3 |
| | adrenal gland, 20M |
| | adrenal gland, 20M, FL-EN, EF |
| | adrenal gland, 8M |
| | aorta, 64M, WN |
| | aorta, adventitia, 65F |
| | bladder and seminal vesicle, 28M |
| | bladder tumor, TC CA, 66M, m/BLADNOT06 |
| | bladder tumor, TC CA, 67M, 5RP |
| | bladder, 78F |
| | bone marrow tumor line, CML/AML, pool, MGC, EF |
| | bone marrow tumor line, SH-SY5Y, neuroblastoma, 4F, t/6-OHDA |
| | bone marrow tumor line, SH-SY5Y, neuroblastoma, F, untreated |
| | bone tumor, sacrum, giant cell tumor, 18F, 5RP, EF |
| | brain line, fetal, 17-18wF, RP, 3′ TIGR |
| | brain tumor line, DU 145, mets prostate CA, 69M, Untx, 5C-FL, EF |
| | brain tumor, anaplastic oligodendroglioma, pool, NORM, CGAP |
| | brain tumor, benign meningioma, 35F, NORM |
| | brain tumor, glioblastoma, pool, NORM, CGAP |
| | brain tumor, mixed types, pool, CGAP |
| | brain, allocortex, cingulate, aw/cholangioCA, 55F, RP |
| | brain, archaeocortex, hippocampus, aw/cholangioCA, 55F, RP |
| | brain, aw/hypoplastic left heart, fetal, 23wM, NORM |
| | brain, aw/muscular atrophy, 3mF, NORM, GEXP |
| | brain, caudate/putamen/nucleus accumbens, aw/CHF, 35M |
| | brain, cerebellum, aw/COPD, 69M |
| | brain, choroid plexus, hemorrhage, 44M |
| | brain, cingulate, aw/MI, 85F, AMP/N |
| | brain, corpus callosum, AD, 74M |
| | brain, dentate nucleus, aw/cholangioCA, 55F, RP |
| | brain, frontal cortex, aw/CHF, 35M |
| | brain, frontal cortex, aw/CHF, 35M, NORM |
| | brain, frontal, gliosis, 46M |
| | brain, frontal, polymicrogyria, gliosis, 5M |
| | brain, globus pallidus/substantia innominata, aw/CA, 55F, RP |
| | brain, hippocampus, aw/CHF, 35M |
| | brain, hippocampus, aw/CHF, 35M, NORM |
| | brain, hippocampus, mw/intracranial hemorrhage, 72F, NORM |
| | brain, medulla, aw/CHF, 35M, AMP |
| | brain, mixed tissues, aw/cholangioCA, 55F, RP |
| | brain, mw/oligoastrocytoma, epilepsy, 26M, NORM |
| | brain, striatum/globus pallidus, AD, aw/mets adenoCA, 70F |
| | brain, temporal cortex, aw/aortic aneurysm, 45F, RP |
| | breast tumor, adenoCA, 46F, m/BRSTNOT17 |
| | breast tumor, adenoCA, 55F, m/BRSTNOT02 |
| | breast tumor, adenoCA, 62F, m/BRSTNOT14 |
| | breast tumor, ductal CA, 65F |
| | breast tumor, ductal, F, pool, NORM, 3′/5′ CGAP |
| | breast tumor, lobular CA, 59F, m/BRSTNOR01, BRSTNOT16 |
| | breast, F, NORM, WM |
| | breast, PF changes, mw/multifocal ductal CA in situ, 46F |
| | breast, mw/ductal adenoCA, 62F, RP |
| | breast, pool, LICR, EF |
| | cervical tumor line, HeLa, adenoCA, 31F, t/5AZA 72 hr |
| | colon line, pool, LICR, EF |
| | colon tumor, CA, pool, LICR, EF |
| | colon tumor, adenoCA, NORM, 3′ CGAP |
| | colon tumor, adenoCA, pool, NORM, 3′ CGAP |
| | colon tumor, rectum, adenoCA, mw/tubular adenoma, 50M, 5RP |
| | colon tumor, sigmoid, adenoCA, 62M, m/COLNNOT16 |
| | colon, cecum polyp, aw/adenoCA, 67F |
| | colon, mucosa, Crohn, pool, NORM, CGAP |
| | coronary artery, smooth muscle cells, 3M, NORM |
| | coronary artery, smooth muscle cells, 3M, t/TNF, IL-1, SUB |
| | epididymis, M, TIGR |
| | fallopian tube tumor, endometrioid/serous adenoCA, 85F |
| | fallopian tube tumor, endometrioid/serous adenoCA, 85F, RP |
| | fat, mesentery, aw/diverticulosis, diverticulitis, 71M |
| | germ cell tumor, pool, NORM, 3′ CGAP |
| | germ cell tumor, pool, SUB, 3′ CGAP |
| | heart, fetal, 18wM |
| | heart, fetal, 19w, NORM, CGAP/WM/WN |
| | heart, left ventricle, mw/myocardial infarction, 56M |
| | iliac artery, endothelial cells, F, t/TNF, IL-1 20 hr |
| | kidney epithelial transf embryo line, 293-EBNA, lg cDNA |
| | kidney epithelial transf embryo line, 293-EBNA, t/5AZA |
| | kidney epithelial transf embryo line, 293-EBNA, t/5AZA, SUB |
| | kidney epithelial transf embryo line, 293-EBNA, tf/E2F1, DP1 |
| | kidney line, 293-EBNA, t/5AZA, Trichostatin A, FL-EN, EF |
| | kidney tumor line, CA/adenoCA/hypernephroma, pool, MGC, EF |
| | kidney tumor, clear cell type cancer, pool, NORM, 3′ CGAP |
| | kidney tumor, renal cell CA, 51F |
| | kidney tumor, renal cell, 3′ CGAP |
| | kidney, 64F |
| | kidney, 8M |
| | kidney, cortex, mw/renal cell CA, 65M |
| | kidney, fetal, 19-23w, M/F, lg cDNA |
| | kidney, right/left, aw/Patau's, fetal, 20wM, pool, lg cDNA |
| | liver tumor line, C3A, Hepatob, 15M, t/APAP-48 hr |
| | liver tumor line, C3A, Hepatob, 15M, t/insulin-24 hr, 5C-FL, EF |
| | liver tumor line, adenoCA, MGC, EF |
| | liver, 4M, FL-EN, EF |
| | liver, aw/Patau's syndrome, fetal, 20wM, 5RP |
| | liver, fetal, M, 5RP |
| | liver, hepatitis C, 35M |
| | liver/spleen, fetal, 20wM, NORM, CGAP/WM/WN |
| | liver/spleen, fetal, 20wM, NORM, WM |
| | lung tumor line, NCI-H69, sm cell CA, 55M, untreated, WM/WN |
| | lung tumor, CA, pool, LICR, EF |
| | lung tumor, neuroendocrine carcinoid, pool, NORM, 3′ CGAP |
| | lung tumor, squamous cell CA, 68M |
| | lung tumor, squamous cell CA, pooled, NORM, CGAP |
| | lung, 17F |
| | lung, 72M |
| | lung, 72M, WM/WN |
| | lung, asthma, 17M |
| | lung, aw/anencephaly, fetal, 20wF |
| | lung, mw/adenoCA, 53M, m/LUNGTUT17 |
| | lung, mw/mets osteoSAR, aw/pleura mets, 58M |
| | lung, mw/spindle cell carcinoid, 15-62M/F, pool, lg cDNA |
| | lymph node tumor line, lymphoma/Burkitt, pool, MGC, EF |
| | lymph node tumor, follicular lymphoma, 3′ CGAP |
| | lymph node, necrotic, aw/lung squamous cell CA, 67M |
| | lymph nodes, 16mM, NORM |
| | lymphocytes, nonactivated Th1 cells |
| | mixed, (melanocytes, uterus, fetal heart), SUB, CGAP/WM/WN |
| | muscle, skeletal, mw/malignant hyperthermia, WM/WN |
| | neuroganglion tumor, ganglioneuroma, 9M |
| | osteogenic tumor line, Saos-2, SAR, 11F, untreated |
| | ovary tumor line, adenoCA, pool, MGC, EF |
| | ovary tumor, CA, F, pool, LICR, EF |
| | ovary tumor, papillary serous CA, 64F, WM/WN |
| | ovary tumor, seroanaplastic CA, 52F |
| | ovary, aw/leiomyomata, 36F |
| | ovary, endometriosis, 24F |
| | pancreas tumor, adenoCA, 3′ CGAP |
| | paraganglion tumor, paraganglioma, aw/renal cell CA, 46M |
| | parathyroid tumor, adenoma, M/F, NORM, WM |
| | parathyroid, hyperplasia, 69F |
| | parotid gland, mw/Warthin's tumor, 70M |
| | periph blood, B-lymphocytes, CLL, pool, NORM, 3′ CGAP |
| | periph blood, blast cells, AML, 58F |
| | periph blood, lymphocytes, non-adher PBMC, M/F, t/LPS |
| | periph blood, monocytes, 42F, t/antiIL-10, LPS, NORM |
| | periph blood, promonocyte line, THP-1, AML, control |
| | pituitary gland, aw/schizophrenia, COPD, 55M |
| | pituitary, 16-70M/F, pool |
| | placenta, aw/hydrocephalus, fetal demise, 16w, 18wM, pool |
| | placenta, neonatal, F, NORM, WM |
| | probable astrocytes, M/F, untreated |
| | prostate stroma, fibroblasts, fetal, 26wM, untreated |
| | prostate tumor line, CA/adenoCA, pool, MGC, EF |
| | prostate tumor, adenoCA, 66M, m/PROSNOT15, PROSDIN01 |
| | prostate, 32M, NORM, 3′/5′ CGAP |
| | prostate, 32M, SUB, 3′ CGAP |
| | prostate, AH, mw/adenoCA, 67M |
| | prostate, AH, mw/adenoCA, M, m/PROSTUT13, PROSTUS08/19/20/25 |
| | prostate, epithelial cells, 17M, untreated |
| | prostate, epithelium, mw/tumor, M, m/PROSTUP02, 3′ CGAP |
| | prostate, pool, M, LICR, EF |
| | seminal vesicle, aw/prostate adenoCA, 63M, 5RP |
| | skin tumor line, melanoma/squamous cell CA, pool, MGC, EF |
| | skin, leg, keratinocytes, neonatal, M |
| | sm intestine tumor, ileum, mets endometrial adenoCA, 64F |
| | sm intestine, 13M |
| | sm intestine, 31F, RP |
| | sm intestine, aw/Patau's syndrome, fetal, 20wM, 5RP |
| | sm intestine, fetal, M, RP |
| | sm intestine, ileum, aw/adenoCA cecum, node mets, 70F, RP |
| | sm intestine, ileum, chronic inflammation, 29F, 5RP, EF |
| | soft tissue tumor, spinal schwannoma, 35M |
| | spleen tumor, malignant lymphoma, 28M, 5RP |
| | spleen, 8M |
| | stomach, 55M |
| | stomach, gastritis, mw/adenoCA, node mets, 76M, RP |
| | teratoCA line, NT2, t/cytokines, 5C-FL, EF |
| | testis tumor, M, TIGR |
| | testis tumor, embryonal CA, 31M, EF |
| | testis tumor, seminoma, 45M |
| | testis, 16M |
| | testis, M, NORM, CGAP/WN |
| | testis, aw/cirrhosis, 37M |
| | thymus, 3M |
| | thymus, aw/patent ductus arteriosus, 3M, 5RP |
| | thymus, fetal, M |
| | thyroid, aw/CHF, 64F |
| | thyroid, lymphocytic thyroiditis, mw/papillary CA, 13F |
| | tonsil, B-lymphocytes, germinal, aw/tonsillitis, NORM, CGAP |
| | uterus tumor, endometrial adenoCA, F, pooled, 3′ CGAP |
| | uterus tumor, serous papillary CA, F, pooled, 3′ CGAP |
| | uterus, F, NORM, CGAP/WM/WN |
| | uterus, endometrium, type I defect, 30, 32, 36F, pool |
| | uterus, mixed, endometrium/myometrium, 32, 45F, pool, RP |
| | uterus, myometrium, mw/leiomyoma, 41F, RP, m/UTRSTUT05 |
| | colon tumor, adenoCA, NORM, SUB, CGAP |
| | adrenal tumor, cortical CA, aw/hilar mets, 52M, RP |
| | brain, aw/hypoplastic left heart, fetal, 23wM, 5RP |
| | brain, corpus callosum, Huntington's, mw/CVA, 57M, RP |
| #50347: | whole embryo, mainly body |
| | whole embryo, mainly head |
| | placenta, choriocarcinoma |
| | ileal mucosa |
| | teratocarcinoma (NT2) |
| | liver tumor, hepatocellular CA, pool, CHGC, EF |
| | breast, pool, LICR, EF |
| | brain line, fetal, 17-18wF, RP, 3′ TIGR |
| | kidney, pool, SUB, 3′ CGAP |
| | lung, idiopathic pulmonary disease, SUB |
| | microvascular, dermal, endothelial cells, 30F, untreated |
| | mixed tissues, fetal lung, testis, B-cell, SUB, 3′ CGAP/WN |
| | osteogenic tumor line, Saos-2, SAR, 11F, untreated |
| | pituitary gland, aw/AD, mets adenoCA, 70F, RP |
| | synovium, wrist, rheuA, 62F |
| | testis, M, NORM, CGAP/WN |
| | adrenal glands, aw/anencephaly, fetal, 16wF, lg cDNA |
| | blood, dendritic cells, mature, pool, CHGC, EF |
| | Jurkat line, T-cell leukemia, M, Untx, 5 C-FL, EF |
| | Jurkat line, T-cell leukemia, untreated, TIGR |
| | UCB, derived dendritic cells, M |
| | UCB, derived dendritic cells, M, t/PMA, Iono-5 hr |
| | UCB, derived dendritic cells, pool, untreated/treated, NORM |
| | adipose, abdomen, aw/obesity, 21M |
| | adrenal gland, 8M |
| | adrenal tumor, cortical CA, aw/hilar mets, 52M, RP |
| | adrenal tumor, pheochromocytoma, 57F |
| | bladder tumor line, CA/TC CA/papilloma, pool, MGC, EF |
| | bladder tumor, TC CA, 58M |
| | bladder, chronic cystitis, aw/urethral adenoCA, 73M |
| | bone, rib, aw/Paget-Schroetter, 57M |
| | bone, rib, aw/Patau's, fetal, 20wM |
| | brain stem, aw/DMt1, 72M, NORM |
| | brain tumor, benign meningioma, 35F |
| | brain tumor, benign meningioma, 35F, 5RP |
| | brain tumor, frontal/parietal, meningioma, 76F |
| | brain tumor, meningioma, 36M |
| | brain, allocortex, cingulate, aw/cholangioCA, 55F, RP |
| | brain, aw/hypoplastic left heart, fetal, 23wM, 5RP |
| | brain, aw/hypoplastic left heart, fetal, 23wM, NORM |
| | brain, cerebellum, Huntington's, mw/CVA, 57M, RP |
| | brain, cerebellum, aw/COPD, left ventricular failure, 70M |
| | brain, frontal cortex, aw/CHF, 35M |
| | brain, frontal, gliosis, mw/epilepsy, cerebral palsy, 27M |
| | brain, hippocampus, AD |
| | brain, hypothalamus, Huntington's, mw/CVA, 57M, NORM |
| | brain, mixed tissues, aw/CHF, 35M, pool, lg cDNA |
| | brain, multiple sclerosis |
| | brain, mw/oligoastrocytoma, epilepsy, 26M |
| | brain, mw/oligoastrocytoma, epilepsy, 26M, NORM |
| | brain, neurogenic tumor line, SK-N-MC, neuroepithelioma, 14F |
| | brain, parietal cortex, aw/CHF, 35M |
| | brain, temporal cortex, aw/aortic aneurysm, 45F |
| | brain, temporal, mw/neuroepithelial tumor, epilepsy, 45M |
| | breast tumor, CA, pool, LICR, EF |
| | breast tumor, adenoCA, 54F, m/BRSTNOT03 |
| | breast tumor, adenoCA, 55F, m/BRSTNOT02 |
| | breast tumor, adenoCA, 62F, m/BRSTNOT14 |
| | breast, 35F |
| | breast, PF changes, mw/ductal adenoCA, 54F, m/BRSTTUT02 |
| | breast, PF changes, mw/intraductal cancer, 48F |
| | bronchial, epithelial cells, 23M, t/20% smoke 20 hr |
| | cervical tumor line, HeLa, adenoCA, 31F, t/5AZA 72 hr |
| | colon tumor, adenoCA, 3′, CGAP |
| | colon tumor, adenoCA, 60M, m/COLNNOT07/08/09/11 |
| | colon tumor, adenoCA, 64F |
| | colon tumor, adenoCA, 65F, EF |
| | colon, ascending, CUC, 74M, EF |
| | colon, cecum polyp, aw/adenoCA, 67F |
| | colon, cecum/descending, polyposis, polyp, M/F, pool, NORM |
| | colon, mixed tissues, 16M/13F, pool, NORM |
| | colon, sigmoid, CUC, 70M |
| | ear, cochlea, fetal, 16-22w, pool, WM |
| | embryo, 8w, TIGR |
| | epiglottis, aw/papillary thyroid CA, 71M |
| | eye, retina, 55M, NORM, WM |
| | fat, mixed tissues, aw/breast adenoCA, 38-73M/F, pool, NORM |
| | femoral artery, aw/chondroSAR, 68M |
| | fetus, 8-9w, pool, NORM, CGAP/WM/WN |
| | fetus, 9w, TIGR |
| | gallbladder, cholecystitis, cholelithiasis, 18F |
| | gallbladder, cholecystitis, cholelithiasis, 53F |
| | gallbladder, cholecystitis, cholelithiasis, 55F, 5RP |
| | ganglion, dorsal root, cervical, aw/lymphoma, 32M, NORM, EF |
| | ganglion, dorsal root, thoracic, aw/lymphoma, 32M |
| | germ cell tumor, pool, SUB, 3′ CGAP |
| | heart, aorta, 10M |
| | heart, fetal, 18wM |
| | heart, fetal, 19w, NORM, CGAP/WM/WN |
| | heart, hypoplastic left, fetal, 23wM |
| | heart, left atrium, 51F |
| | heart, right atrium, 51F |
| | iliac artery, endothelial cells, F, t/1% oxygen 24 hr |
| | kidney, 2dF |
| | liver tumor line, C3A, Hepatob, 15M, Untx |
| | liver tumor line, adenoCA, MGC, EF |
| | liver tumor, adenoma, CGAP |
| | liver tumor, mets neuroendocrine CA, 62F, m/ LIVRTMR01 |
| | liver/spleen, fetal, 20wM, NORM, WM |
| | lung, 15F |
| | lung, 2M |
| | lung, asthma, 17M |
| | lung, aw/anencephaly, fetal, 17wF |
| | lung, fetal, 19w, NORM, CGAP/WM/WN |
| | lung, idiopathic pulmonary disease, NORM |
| | lung, mw/adenoCA, COPD, 47M |
| | lung, mw/mets thyroid CA, 79M, m/LUNGTUT02 |
| | lymph node tumor, follicular lymphoma, 3′ CGAP |
| | lymph node, 11F |
| | lymph node, 16mM |
| | lymph node, B-lymphocytes, germinal center, pool, MGC, EF |
| | microvascular, dermal, endothelial cells, neonatal, M |
| | mixed tissue tumor, head/neck, CA, pool, LICR, EF |
| | mixed tissues, includes tumor, SUB, 3′ CGAP |
| | mixed tissues, includes tumor, treated cells, pool, NORM, EF |
| | mixed, (melanocytes, uterus, fetal heart), SUB, CGAP/WM/WN |
| | mouth line, gingiva, keratinocytes, CGAP, EF |
| | multiple sclerosis, 46M, NORM, WM/WN |
| | muscle, forearm, mw/intramuscular hemangioma 38F |
| | omentum tumor, mets ovary papillary serous CA, F, CGAP |
| | ovary tumor, TC CA, 53F |
| | ovary tumor, fibrothecoma, F, pool, NORM, 3′, CGAP |
| | ovary tumor, mets colon adenoCA, 58F |
| | ovary tumor, mucinous cystadenoCA, 43F, m/OVARNOT03 |
| | ovary, aw/cardiomyopathy, 59F, NORM |
| | ovary, endometriosis, aw/leiomyomata, 39F, NORM, EF |
| | ovary, follicular cysts, 28F |
| | pancreas, 8M, 5RP |
| | parathyroid tumor, adenoma, M/F, NORM, WM |
| | penis, corpora cavernosa, M |
| | penis, corpus cavernosum, M |
| | periph blood, promonocyte line, THP-1, AML, t/5AZA |
| | periph blood, promonocyte line, THP-1, AML, t/PMA, LPS |
| | periph blood, promonocyte line, THP-1, AML, untreated |
| | placenta tumor line, chorioCA, fetal, pool, MGC, EF |
| | placenta, aw/hydrocephalus, fetal, 16w, RP |
| | placenta, fetal, 8-9w, pool, NORM, CGAP/WM |
| | placenta, neonatal, F, NORM, WM |
| | prostate tumor, adenoCA, 57M, m/PROSNOT06 |
| | prostate tumor, adenoCA, 61M |
| | prostate, 21M, TIGR |
| | prostate, AH, aw/bladder TC CA, 58M |
| | prostate, AH, mw/adenoCA, 65M |
| | prostate, AH, mw/adenoCA, 66M, NORM, m/PROSTUT10 |
| | prostate, AH, mw/adenoCA, node mets, 55M, lg/N, m/PROSTUT16 |
| | pulmonary artery, endothelial cells, 10M, t/TNF, IL-1 |
| | renal vein, smooth muscle cells, 57M, Untx |
| | skin, foreskin, melanocytes, M, NORM, WM/WN |
| | skin, pool, CGAP, EF |
| | sm intestine, duodenum tumor line, adenoCA, MCG, EF |
| | sm intestine, ileum, mw/CUC, 25F |
| | spinal cord, aw/renal failure, 71M |
| | spinal cord, aw/renal failure, 71M, NORM |
| | spleen, Gaucher's, 22M |
| | stomach tumor, CA, pool, LICR, EF |
| | stomach, aw/esophagus adenoCA, 61M, 5RP |
| | stomach, pool, LICR, EF |
| | synovium, knee, OA, 82F |
| | teratoCA line, NT2, t/cytokines, 5C-FL, EF |
| | teratoCA line, NT2, t/cytokines, NORM, EF |
| | teratoCA line, hNT2, t/RA + MI |
| | teratoCA line, hNT2, untreated |
| | testis tumor line, embryonal CA, pool, MGC, EF |
| | testis, 10-61M, pool, lg cDNA |
| | testis, 16M |
| | testis, aw/cirrhosis, 37M |
| | thymus, hyperplasia, aw/myasthenia gravis, 16F |
| | tonsil, B-lymphocytes, germinal, aw/tonsillitis, NORM, CGAP |
| | umb cord blood, mononuclear cells, t/IL-5 |
| | uterus tumor, endometrial adenoCA, F, pooled, 3′ CGAP |
| | uterus tumor, leiomyoma, 37F, 5RP |
| | uterus tumor, serous papillary CA, F, pooled, 3′ CGAP |
| | uterus, F, NORM, CGAP/WM/WN |
| | uterus, endometrium, aw/cystocele, 38F |
| | uterus, endometrium, mw/cervical dysplasia, 32F |
| | uterus, endometrium, type I defect, 28F |
| | uterus, endometrium, type II defect, endometriosis, F |
| | uterus, myometrium, mw/leiomyoma, 41F, NORM, m/UTRSTUT05 |
| | uterus, myometrium, mw/leiomyoma, 41F, RP, m/UTRSTUT05 |
| | uterus, myometrium, mw/leiomyoma, 41F, m/UTRSTUT05 |
| #689869: | UCB, CD34+ hematopoietic stem cells, CHGC, EF |
| | adrenal glands, aw/anencephaly, fetal, 16wF |
| | aorta, adventitia, 65F |
| | bone marrow tumor line, CML/AML, pool, MGC, EF |
| | bone tumor/line, MG-63, osteoSAR/giant cell, M/F, pool, RP, EF |
| | bone, rib, aw/Patau's, fetal, 20wM |
| | bone, rib, aw/Patau's, fetal, 20wM, lg cDNA |
| | brain tumor, frontal, mets hypernephroma, 58M |
| | brain, infant, 10wF, NORM, WM |
| | brain, mw/oligoastrocytoma, epilepsy, 26M, NORM |
| | brain, substantia nigra, aw/atherosclerosis, CHF, 81F, NORM |
| | breast, 46F |
| | breast, F, NORM, WM |
| | breast, mw/ductal CA, CA in situ, aw/node mets, 62F |
| | colon, 25M, WN |
| | fetus, 8-9w, pool, NORM, CGAP/WM/WN |
| | ganglion, dorsal root, 36M, CGAP |
| | ganglion, dorsal root, CGAP |
| | ganglion, dorsal root, thoracic, aw/lymphoma, 32M, NORM |
| | heart, fetal, 19w, NORM, CGAP/WM/WN |
| | heart, fetal, 8-10w, pool, BI |
| | heart, left atrium, 51F |
| | heart, right atrium/muscle wall, Pompe's, 7mM, RP |
| | kidney, pool, NORM, 3′ CGAP |
| | liver, aw/Patau's, anencephaly, fetal, pool, lg cDNA |
| | lung tumor, myxoid lipoSAR, 65F |
| | lung, 12M |
| | lung, 72M, WM/WN |
| | lung, aw/Patau's, fetal, 20wM |
| | lung, fetal, 19w, NORM, CGAP/WM/WN |
| | lung, idiopathic pulmonary disease, SUB |
| | lung, mw/adenoCA, 63M |
| | lung, pleura, aw/mets leiomyoSAR to lung, 58F, lg cDNA |
| | lymph nodes, 16mM, NORM |
| | mixed tissues, includes tumor, 23-66M, pool, NORM, EF |
| | mixed, (melanocytes, uterus, fetal heart), SUB, CGAP/WM/WN |
| | muscle tumor, striated, alveolar rhabdomyoSAR, 3′/5′ CGAP |
| | muscle, psoas, 12M |
| | nose, nasal polyps |
| | ovary, aw/cardiomyopathy, 59F |
| | ovary, endometriosis, aw/leiomyomata, 39F |
| | paraganglion tumor, paraganglioma, aw/renal cell CA, 46M |
| | placenta, fetal, 21wF |
| | placenta, fetal, TIGR |
| | placenta, neonatal, F, NORM, WM |
| | stomach tumor, adenoCA, poorly differentiated, 3′ CGAP |
| | synovium, wrist, rheuA, 56F |
| | testis, 16M, NORM |
| | testis, necrosis, 31M |
| | thyroid, aw/CHF, 64F |
| | umb cord vein endothelial line, HUV-EC-C, shear stress |
| | umbilical vein, endothelial cells, pool, WM/WN |
| | uterus tumor, endometrial adenoCA, F, pooled, 3′ CGAP |
| | uterus tumor, serous papillary CA, F, pooled, 3′ CGAP |
| | uterus, F, NORM, CGAP/WM/WN |
| | uterus, aw/cardiomyopathy, 59F |
| | uterus, endometrium, mw/cervical dysplasia, 32F, FL-EN, EF |
| | uterus, myometrium, mw/multiple leiomyomata, 45F |
| | brain, allocortex/neocortex, cingulate, aw/CA, 55F, RP |
| | chest wall, soft tissue, mw/adenoCA, aw/COPD, 63M |
| | microvascular, dermal, endothelial cells, 22F, t/bFGF, EF |
| | placenta, aw/hydrocephalus, fetal, 16w |
| | skin, leg, erythema nodosum |
| | sm intestine, fetal, 8-16M/F, pool, NORM |
| | sm intestine, mw/carcinoid, aw/node mets, 59M |
| | stomach, fetal, 18wM |
| | colon tumor, adenoCA, 3′ CGAP |
| | liver, 4M, FL-EN, EF |
| | lung tumor, CA, pool, LICR, EF |
| | ovary tumor, endometrioid CA, 62F |
| | spleen, 2M, 5RP, EF |
| | uterus tumor, leiomyomata/adenosquamousCA, F, pool, lg cDNA, EF |
| | adipose, abdomen, aw/obesity, 21M |
| | adrenal gland, aw/pituitary neoplasm, 61F |
| | adrenal tumor, mets renal cell CA, 50M |
| | aorta, adventitia, 48M |
| | aorta, endothelial cells, M |
| | bladder tumor, microscopic foci TC CA, 58M |
| | bladder, mw/TC CA, aw/node mets, 58M, m/BLADTUT03 |
| | bladder, mw/TC CA, aw/prostate TC CA, 66M, m/BLADTUT05 |
| | bone marrow, CD34+ hematopoietic stem cells, pool, CHGC, EF |
| | bone tumor, rib, mets osteoSAR, 16M |
| | brain tumor, frontal, meningioma, 68M |
| | brain tumor, frontal, neuronal neoplasm, 32M |
| | brain tumor, meningioma, 36M |
| | brain, aw/hypoplastic left heart, fetal, 23wM, FL-EN, EF |
| | brain, hypothalamus, Huntington's, mw/CVA, 57M |
| | brain, mixed tissues, aw/cholangioCA, 55F, RP |
| | brain, temporal cortex, aw/aortic aneurysm, 45F |
| | breast tumor, CA, pool, LICR, EF |
| | breast tumor, adenoCA, 46F, m/BRSTNOT17 |
| | breast tumor, adenoCA, 54F, m/BRSTNOT03 |
| | breast tumor, adenoCA, 55F, m/BRSTNOT02 |
| | breast, 46, 60F, pool, NORM |
| | breast, NF changes, mw/ductal adenoCA, 40-57F, pool, lg cDNA |
| | breast, PF breast disease, 57F |
| | breast, PF changes, 40F |
| | breast, PF changes, mw/adenoCA, 55F, m/BRSTTUT01 |
| | breast, PF changes, mw/adenoCA, intraductal CA, 43F |
| | breast, PF changes, mw/ductal adenoCA, 54F, m/BRSTTUT02 |
| | breast, mw/ductal adenoCA, aw/node mets, 46F, m/BRSTTUT15 |
| | breast, mw/ductal adenoCA, intraductal CA, aw/node mets, 57F |
| | breast, mw/lobular CA, 67F |
| | breast, papillomatosis, mw/lobular CA, 59F, m/BRSTTUT22 |
| | colon tumor, adenoCA, 65F, EF |
| | colon tumor, adenoCA, pool, NORM, 3′ CGAP |
| | colon, cecum, Crohn's, 31M |
| | colon, cecum, benign familial polyposis, 16M |
| | colon, descending, benign familial polyposis, 16M |
| | colon, mw/adenoCA, aw/node mets, 60M, m/COLNTUT16 |
| | colon, normal/pseudopolyp, Crohn, l6, 26M, pool, lg/N |
| | colon, transverse, Crohn's, 26M |
| | colon, ulcerative colitis, 16M |
| | epiglottis, aw/papillary thyroid CA, 71M |
| | esophagus tumor, adenoCA, 61M, 5RP, EF |
| | fat, abdomen, aw/obesity, 52F |
| | fat, axillary, aw/breast adenoCA, 73F |
| | fat, mesentery, aw/diverticulosis, diverticulitis, 71M |
| | femoral artery, aw/chondroSAR, 68M |
| | gallbladder, cholecystitis, cholelithiasis, 21M |
| | ganglion, dorsal root, cervical, aw/lymphoma, 32M, NORM, EF |
| | germ cell tumor, pool, SUB, 3′ CGAP |
| | heart, aorta, 10M |
| | heart, aorta, 39M, 5RP |
| | heart, coronary artery, CAD, 46M |
| | heart, coronary artery, endothelial cells, 58M |
| | heart, left ventricle, 39M |
| | heart, right atrium/muscle wall, Pompe's, 7mM |
| | iliac artery, endothelial cells, F, control, untreated |
| | kidney, 2dF |
| | kidney, 8M |
| | kidney, MGC, EF |
| | kidney, aw/hypoplastic left heart, fetal, 23wM |
| | kidney, cortex, mw/renal cell CA, 65M |
| | kidney, mw/renal cell CA, 8, 53F, pool, NORM |
| | kidney, pool, SUB, 3′ CGAP |
| | liver tumor, mets neuroendocrine CA, 72M, 5RP, EF |
| | liver/spleen, fetal, 20wM, NORM, WM |
| | lung tumor, pleura, mets uterine leiomyoSAR, 55F |
| | lung tumor, squamous cell CA, 57M |
| | lung tumor, squamous cell CA, 65F |
| | lung, 72M |
| | lung, asthma, 8F |
| | lung, aw/Patau's, fetal, 20wM, lg cDNA |
| | lung, aw/polycystic kidneys, fetal, 23wM |
| | lung, fetal, 23wM |
| | lung, mw/adenoCA, 53M, m/LUNGTUT17 |
| | lung, mw/endobronchial carcinoid, 33M |
| | lung, pneumonitis, mw/squamous cell CA, 69M, m/LUNGTUT03 |
| | lymph node, necrotic, aw/lung squamous cell CA, 67M |
| | lymph nodes, 14F |
| | mast cells, liver, fetal, 22w |
| | megakaryoblast line, MEG-01, CML, 55M, untreated |
| | meniscus, tibial, aw/mets alveolar rhabdomyoSAP, 16M |
| | microvascular, dermal, endothelial cells, 22F, Untx |
| | microvascular, dermal, endothelial cells, 22F, t/VEGF, EF |
| | mixed tissues, fetal lung, testis, B-cell, SUB, 3′ CGAP/WN |
| | muscle, forearm, mw/intramuscular hemangioma 38F |
| | muscle, tibial, aw/thrombosis, 41F |
| | neuroganglion tumor, ganglioneuroma, 9M |
| | nose, nasal polyps, aw/asthma, 78M, pool, NORM |
| | nose, nasal polyps, lg cDNA, EF |
| | ovary tumor, mucinous cystadenoCA, 43F, m/OVARNOT03 |
| | ovary tumor, papillary serous cystadenoCA 36F, NORM, WM/WN |
| | ovary, aw/leiomyomata, 36F, NORM |
| | ovary, endometriosis, aw/leiomyomata, 39F, NORM, EF |
| | ovary, endometriosis, aw/multiple leiomyomata, 47F, 5C-RP |
| | pancreas, islet cell hyperplasia, 15M |
| | pancreatic tumor, anaplastic CA, 45F |
| | penis, corpora cavernosa, M |
| | penis, corpora cavernosa, aw/scrotal urothelial CA, M |
| | penis, corpus cavernosum, M |
| | placenta, aw/hydrocephalus, fetal demise, 16w, 18wM, pool |
| | placenta, fetal, 18wM |
| | placenta, fetal, 8-9w, pool, NORM, CGAP/WM |
| | placenta, pool, LICR, EF |
| | prostate tumor line, CA/adenoCA, pool, MGC, EF |
| | prostate tumor, adenoCA, 59M, SUB, m/PROSNOT19 |
| | prostate, 32M, SUB, 3′ CGAP |
| | prostate, AH, mw/adenoCA, 53M |
| | prostate, AH, mw/adenoCA, 55M, m/PROSTUT16 |
| | skin, breast and fetal, aw/intraductal CA, pool |
| | skin, breast, 17F, RP |
| | skin, breast, 26F |
| | skin, breast, aw/adenoCA, 70F, lg cDNA |
| | sm intestine, 13M |
| | sm intestine, 15F |
| | sm intestine, aw/stomach ulcer, 49F, 5RP |
| | sm intestine, fetal, 20wF |
| | sm intestine, ileum, Crohn's, 18F |
| | sm intestine, jejunum, 8F |
| | smooth muscle tumor, CA/leiomyoSAR, pool, LICR, EF |
| | soft tissue tumor, thigh, mets myxoid lipoSAR, 34F |
| | spleen tumor, malignant lymphoma, 28M, 5RP |
| | spleen, 2M |
| | spleen, fetal, 23wM |
| | stomach, gastritis, mw/adenoCA, node mets, 76M, RP |
| | synovium, wrist, dorsal, rheuA, 64F |
| | testis tumor, embryonal CA, 31M, 5RP |
| | testis tumor, seminoma, 45M |
| | testis, 16M |
| | testis, M, NORM, CGAP/WN |
| | thymus, aw/Down, 4mM |
| | thymus, aw/patent ductus arteriosus, 3M, 5RP |
| | thyroid tumor, follicular adenoma, 17M |
| | thyroid, lymphocytic thyroiditis, mw/papillary CA, 30F |
| | tibia, periosteum, mw/osteoSAR, osteogenesis imperfecta, 20M |
| | tonsil, lymphoid hyperplasia, 6M |
| | ureter tumor, TC CA, 69M |
| | uterus, aw/ovarian follicular cysts, 34F |
| | uterus, cervix, 40F |
| | uterus, endometrium, mw/cervical dysplasia, 32F |
| | uterus, endometrium, type II defect, endometriosis, F |
| | uterus, myometrium, mw/adenoCA, 59F, RP |

TABLE 2
List of PFAM domains found in known protein kinases
that have been identified in novel kinases.

| Gene | Domain | p-value | from | to |
|---|---|---|---|---|
| 33751_{—} | PH | 1.1e-17 | 1144 | 1246 |
| 33890_{—} | PI3K_rbd | 2.1e-46 | 204 | 313 |
| 33890_{—} | PI3Ka | 6e-105 | 542 | 734 |
| 33890_{—} | PI3_PI4_kinase | 1.6e-112 | 828 | 1045 |
| 36832_{—} | PH | 5e-14 | 421 | 612 |
| 36832_{—} | ank | 6e-09 | 792 | 824 |
| 36832_{—} | ank | 6e-09 | 825 | 849 |
| 36909_{—} | PH | 1.7e-17 | 87 | 189 |
| 38149_{—} | MAM | 1.1e-111 | 66 | 229 |
| 38149_{—} | MAM | 1.1e-111 | 236 | 382 |
| 38149_{—} | MAM | 1.1e-111 | 387 | 410 |
| 38149_{—} | MAM | 1.1e-111 | 428 | 521 |
| 38149_{—} | vwd | 6e-149 | 1141 | 1293 |
| 38149_{—} | vwd | 6e-149 | 1490 | 1645 |
| 38149_{—} | vwd | 6e-149 | 1873 | 1989 |
| 38149_{—} | vwd | 6e-149 | 2231 | 2328 |
| 40497_{—} | Ald_Xan_dh_C2 | 0 | 724 | 1264 |
| 40980_{—} | PIP5K | 3e-163 | 198 | 447 |
| 40980_{—} | PIP5K | 3e-163 | 460 | 503 |
| 40994_{—} | ubiquitin | 0.687585 | 72 | 114 |
| 41437_{—} | TPR | 0.371773 | 1653 | 1686 |
| 41822_{—} | FAT | 7.7e-157 | 858 | 893 |
| 41822_{—} | FAT | 7.7e-157 | 896 | 1190 |
| 41822_{—} | FAT | 7.7e-157 | 1191 | 1303 |
| 41822_{—} | PI3_PI4_kinase | 9.8e-93 | 1502 | 1541 |
| 41822_{—} | PI3_PI4_kinase | 9.8e-93 | 1545 | 1708 |
| 43542_{—} | PH | 4.9e-07 | 1 | 77 |
| 47701_{—} | RIO1 | 2.5e-81 | 290 | 495 |
| 49904_{—} | WD40 | 8.7e-18 | 48 | 74 |
| 49904_{—} | WD40 | 8.7e-18 | 1761 | 1773 |
| 49904_{—} | WD40 | 8.7e-18 | 1852 | 1890 |
| 49904_{—} | WD40 | 8.7e-18 | 1894 | 1932 |
| 49904_{—} | WD40 | 8.7e-18 | 1946 | 1980 |
| 50258_{—} | WD40 | 7.4e-87 | 1683 | 1707 |
| 50258_{—} | WD40 | 7.4e-87 | 1712 | 1748 |
| 50258_{—} | WD40 | 7.4e-87 | 1752 | 1790 |
| 50258_{—} | WD40 | 7.4e-87 | 1807 | 1831 |
| 50258_{—} | WD40 | 7.4e-87 | 1835 | 1873 |
| 50258_{—} | WD40 | 7.4e-87 | 1877 | 1915 |
| 50258_{—} | WD40 | 7.4e-87 | 1935 | 1958 |
| 50258_{—} | WD40 | 7.4e-87 | 1974 | 1999 |
| 50258_{—} | WD40 | 7.4e-87 | 2005 | 2041 |
| 50258_{—} | WD40 | 7.4e-87 | 2054 | 2092 |
| 50258_{—} | WD40 | 7.4e-87 | 2109 | 2137 |
| 50258_{—} | WD40 | 7.4e-87 | 2141 | 2177 |
| 50258_{—} | WD40 | 7.4e-87 | 2180 | 2227 |
| 50258_{—} | WD40 | 7.4e-87 | 2231 | 2269 |
| 50258_{—} | WD40 | 7.4e-87 | 2283 | 2311 |
| 50258_{—} | WD40 | 7.4e-87 | 2363 | 2389 |
| 50258_{—} | WD40 | 7.4e-87 | 2363 | 2389 |
| 50258_{—} | WD40 | 7.4e-87 | 2548 | 2584 |
| 50347_{—} | SAM | 4.6e-13 | 664 | 726 |
| 51594_{—} | death | 1e-30 | 3627 | 3710 |
| 604530 | PIP5K | 1.1e-155 | 150 | 437 |
| 614552 | Gag_p30 | 2.9e-10 | 21 | 104 |
| 718872 | PX | 0.123445 | 280 | 342 |
| 718872 | PX | 0.123445 | 354 | 368 |
| 721594 | ig | 2.3e-08 | 12 | 95 |
| 721594 | ig | 2.3e-08 | 134 | 201 |
| 721594 | Gag_p30 | 5.2e-07 | 296 | 335 |
| 730817 | PX | 0.077548 | 277 | 345 |
| 730817 | PX | 0.077548 | 351 | 365 |

What is claimed is:

1. An isolated, enriched or purified nucleic acid molecule encoding a kinase polypeptide, wherein said nucleic acid molecule comprises a nucleotide sequence that:
(a) encodes a polypeptide having an amino acid sequence selected from the group consisting of those set forth in SEQ ID NO:1-87;
(b) is the complement of the nucleotide sequence of (a);
(c) hybridizes under stringent conditions to the nucleotide molecule of (a) and encodes a naturally occurring kinase polypeptide;
(d) encodes a polypeptide having an amino acid sequence selected from the group consisting of those set forth in SEQ ID NO:1-87, except that it lacks one or more, but not all, of an N-terminal domain, a C-terminal catalytic domain, a catalytic domain, a C-terminal domain, a coiled-coil structure region, a proline-rich region, a spacer region and a C-terminal tail; or
(e) is the complement of the nucleotide sequence of (d).

2. The nucleic acid molecule of claim 1, further comprising a vector or promoter effective to initiate transcription in a host cell.

3. The nucleic acid molecule of claim 1, wherein said nucleic acid molecule is isolated, enriched, or purified from a mammal.

4. The nucleic acid molecule of claim 3, wherein said mammal is a human.

5. The nucleic acid probe of claim 1 used for the detection of nucleic acid encoding a kinase polypeptide in a sample, wherein said kinase polypeptide is selected from the group consisting of a kinase polypeptide having an amino acid sequence selected from the group consisting of those set forth in SEQ ID NO:1-87.

6. A recombinant cell comprising the nucleic acid molecule of claim 1 encoding a kinase polypeptide having an amino acid sequence selected from the group consisting of those set forth in SEQ ID NO:1-87.

7. An isolated, enriched, or purified kinase polypeptide, wherein said polypeptide comprises an amino acid sequence having
(a) an amino acid sequence selected from the group consisting of those set forth in SEQ ID NO:1-87, respectively;
(b) an amino acid sequence selected from the group consisting of those set forth in SEQ ID NO:1-87, respectively, except that it lacks one or more, but not all, of the domains selected from the group consisting of an N-terminal domain, a C-terminal catalytic domain, a catalytic domain, a C-terminal domain, a coiled-coil structure region, a proline-rich region, a spacer region, and a C-terminal tail.

8. The kinase polypeptide of claim 7, wherein said polypeptide is isolated, purified, or enriched from a mammal.

9. The kinase polypeptide of claim 8, wherein said mammal is a human.

10. An antibody or antibody fragment having specific binding affinity to a kinase polypeptide or to a domain of said polypeptide, wherein said polypeptide is a kinase polypeptide having an amino acid sequence selected from the group consisting of those set forth in SEQ ID NO:1-87.

11. A hybridoma which produces an antibody having specific binding affinity to a kinase polypeptide having an amino acid sequence selected from the group consisting of those set forth in SEQ ID NO:1-87.

12. A kit comprising an antibody which binds to a polypeptide of claim 7 or 8 and negative control antibody.

13. A method for identifying a substance that modulates the activity of a kinase polypeptide comprising the steps of:
(a) contacting the kinase polypeptide having an amino acid sequence selected from the group consisting of those set forth in SEQ ID NO:1-87 with a test substance;
(b) measuring the activity of said polypeptide; and
(c) determining whether said substance modulates the activity of said polypeptide.

14. A method for identifying a substance that modulates the activity of a kinase polypeptide in a cell comprising the steps of:
(a) expressing a kinase polypeptide having an amino acid sequence selected from the group consisting of those set forth in SEQ ID NO:1-87;
(b) adding a test substance to said cell; and
(c) monitoring a change in cell phenotype or the interaction between said polypeptide and a natural binding partner.

15. A method for treating a disease or disorder by administering to a patient in need of such treatment a substance that modulates the activity of a kinase having an amino acid sequence selected from the group consisting of those set forth in SEQ ID NO:1-87.

16. The method of claim 15, wherein said disease or disorder is selected from the group consisting of cancers, immune-related diseases and disorders, cardiovascular disease, brain or neuronal-associated diseases, and metabolic disorders.

17. The method of claim 15, wherein said disease or disorder is selected from the group consisting of cancers of tissues; cancers of hematopoietic origin; diseases of the central nervous system; diseases of the peripheral nervous system; Alzheimer's disease; Parkinson's disease; multiple sclerosis; amyotrophic lateral sclerosis; viral infections; infections caused by prions; infections caused by bacteria; infections caused by fungi; and ocular diseases.

18. The method of claim 15, wherein said disease or disorder is selected from the group consisting of migraines; pain; sexual dysfunction; mood disorders; attention disorders; cognition disorders; hypotension; hypertension; psychotic disorders; neurological disorders; dyskinesias; metabolic disorders; and organ transplant rejection.

19. The method of claim 15, wherein said substance modulates kinase activity in vitro.

20. The method of claim 19, wherein said substance is a kinase inhibitor.

21. A method for detection of a kinase polypeptide in a sample as a diagnostic tool for a disease or disorder, wherein said method comprises:
(a) contacting said sample with a nucleic acid probe which hybridizes under hybridization assay conditions to a nucleic acid target region of a kinase polypeptide having an amino acid sequence selected from the group consisting of those set forth in SEQ ID NO:1-87, said probe comprising the nucleic acid sequence encoding said polypeptide, fragments thereof, or the complements of said sequences and fragments; and
(b) detecting the presence or amount of the probe:target region hybrid as an indication of said disease.

22. The method of claim 21, wherein said disease or disorder is selected from the group consisting of cancers, immune-related diseases and disorders, cardiovascular disease, brain or neuronal-associated diseases, and metabolic disorders.

23. The method of claim 21, wherein said disease or disorder is selected from the group consisting of cancers of tissues; cancers of hematopoietic origin; diseases of the central nervous system; diseases of the peripheral nervous system; Alzheimer's disease; Parkinson's disease; multiple sclerosis; amyotrophic lateral sclerosis; viral infections; infections caused by prions; infections caused by bacteria; infections caused by fungi; and ocular diseases.

24. The method of claim 21, wherein said disease or disorder is selected from the group consisting of migraines, pain; sexual dysfunction; mood disorders; attention disorders; cognition disorders; hypotension; hypertension; psychotic disorders; neurological disorders; dyskinesias; metabolic disorders; and organ transplant rejection.

25. A method for detection of a kinase polypeptide in a sample as a diagnostic tool for a disease or disorder, wherein said method comprises:
(a) comparing a nucleic acid target region encoding said kinase polypeptide in a sample, wherein said kinase polypeptide has an amino acid sequence selected from the group consisting of those set forth in SEQ ID NO:1-87, or one or more fragments thereof, with a control nucleic acid target region encoding said kinase polypeptide, or one or more fragments thereof; and
(b) detecting differences in sequence or amount between said target region and said control target region, as an indication of said disease or disorder.

26. The method of claim 25, wherein said disease or disorder is selected from the group consisting of cancers, immune-related diseases and disorders, cardiovascular disease, brain or neuronal-associated diseases, and metabolic disorders.

27. The method of claim 25, wherein said disease or disorder is selected from the group consisting of cancers of tissues; cancers of hematopoietic origin; diseases of the central nervous system; diseases of the peripheral nervous system; Alzheimer's disease; Parkinson's disease; multiple sclerosis; amyotrophic lateral sclerosis; viral infections; infections caused by prions; infections caused by bacteria; infections caused by fungi; and ocular diseases.

28. The method of claim 25, wherein said disease or disorder is selected from the group consisting of migraines, pain; sexual dysfunction; mood disorders; attention disorders; cognition disorders; hypotension; hypertension; psychotic disorders; neurological disorders; dyskinesias; metabolic disorders; and organ transplant rejection.

29. A method for identifying a remote polypeptide homologue to a referent protein family, comprising:
(a) identifying the conserved secondary structure pattern (CSSP) of said protein family;
(b) identifying the conserved amino acid residues (CAAR) or conserved active site amino acid residues (CASAAR) of the referent protein family; and
(c) identifying the conserved residues embedded into the secondary structure pattern (CRISSP); and
(d) identifying the candidate as a remote homologue if the candidate polypeptide contains the CRISSP of (c).

30. A computer readable medium having program code stored thereon for identifying a remote polypeptide homologue to a referent protein family, the program code configured to cause a computer to perform the following steps:
(a) identifying the conserved secondary structure pattern of said protein family;
(b) identifying the conserved amino acid residues or conserved active site amino acid residues of the referent protein family; and
(c) identifying the conserved residues embedded into the secondary structure pattern (CRISSP); and
(d) identifying the candidate as a remote homologue if the candidate polypeptide contains the CRISSP of (c).

31. A programmed storage device comprising instructions that when executed perform the steps of:
(a) identifying-the conserved secondary structure pattern of said protein family;
(b) identifying the conserved amino acid residues or conserved active site amino acid residues of the referent protein family; and
(c) identifying the conserved residues embedded into the secondary structure pattern (CRISSP); and
(d) identifying the candidate as a remote homologue if the candidate polypeptide contains the CRISSP of (c).

32. A process for effecting analysis of a polypeptide sequence through use of a computer having a memory, said process comprising:
(a) placing into said memory data representing a polypeptide,
(b) developing within said memory a data structure associated with said data and reflecting the underlying organization and structure of the data to facilitate program access to data elements corresponding to logical sub-components of the sequence,
(c) programming said computer with a program containing instructions sufficient to implement the method of claim 29 and
(d) executing said program on said computer while granting said program access to said data and to said data structure within said memory.

33. An isolated, enriched or purified nucleic acid molecule consisting essentially of about 10-30 contiguous nucleotide bases of a nucleic acid sequence that encodes a polypeptide that is selected from the group consisting of SEQ ID NO:88-174.

34. The isolated, enriched or purified nucleic acid molecule of claim 33 consisting essentially of about 15-25 contiguous nucleotide bases of a nucleic acid sequence selected from the group consisting of SEQ ID NO:88-174.
